(19) United States
(12) Patent Application Publication
(10) Pub. No.: US 2024/0352021 A1
(43) Pub. Date: Oct. 24, 2024

(54) COVALENT EGFR INHIBITORS AND METHODS OF USE THEREOF

(71) Applicants: DANA-FARBER CANCER INSTITUTE, INC., BOSTON, MA (US); THE BOARD OF TRUSTEES OF THE LELAND STANFORD JUNIOR UNIVERSITY, STANFORD, CA (US); SPRINGWORKS THERAPEUTICS INC, Stamford, CT (US)

(72) Inventors: JIANWEI CHE, SHARON, MA (US); ZHENGNIAN LI, STANFORD, CA (US); NATHANAEL S. GRAY, STANFORD, CA (US); TINGHU ZHANG, BROOKLINE, MA (US); STEPHEN LEYCESTER GWALTNEY, II, STAMFORD, CT (US); TYLER BEYETT, BROOKLINE, MA (US)

(21) Appl. No.: 18/701,077

(22) PCT Filed: Oct. 18, 2022
(86) PCT No.: PCT/US2022/078318

§ 371 Date: Apr. 12, 2024

Related U.S. Application Data
(60) Provisional application No. 63/364,116, filed on May 4, 2022.
(60) Provisional application No. 63/262,659, filed on Oct. 18, 2021.

(51) Int. Cl. C07D 487/04; A61K 31/519; A61K 31/529; A61K 31/549; A61K 31/551; A61P 35/00; C07D 471/04; C07D 487/18; C07D 519/00

(52) U.S. Cl. CPC C07D 487/04; A61K 31/519; A61K 31/529; A61K 31/549; A61K 31/551; A61P 35/00; C07D 471/04; C07D 487/18; C07D 519/00

(57) ABSTRACT

The disclosure relates to compounds that act as inhibitors of epidermal growth factor receptor (EGFR); pharmaceutical compositions comprising the compounds; and methods of treating or preventing kinase-mediated disorders, including cancer and other proliferation diseases.

### RELATED APPLICATIONS

This application claims priority to U.S. Provisional Application No. 63/262,659 filed on Oct. 18, 2021, and U.S. Provisional Application No. 63/364,116 filed on May 4, 2022, the entire contents of which are hereby incorporated by reference in their entirety.

### BACKGROUND

The epidermal growth factor receptor (EGFR, Erb-B1) belongs to a family of receptor tyrosine kinases that mediate the proliferation, differentiation, and survival of normal and malignant cells (Arteaga, C. L., J. Clin. Oncol. 19, 2001, 32-40). Deregulation of EGFR has been implicated in many types of human cancer, with overexpression of the receptor present in at least 70% of human cancers (Seymour, L. K., Curr. Drug Targets 2, 2001, 117-133), including non-small lung cell carcinomas, breast cancers, gliomas, squamous cell carcinomas of the head and neck, and prostate cancer (Raymond, E., et al., Drugs 60 (Suppl. 1), 2000, 15-23, discussion 41-2; Salomon, D. S., et al., Crit. Rev. Oncol. Hematol. 19, 1995, 183-232; Voldborg B. R., et al., Ann. Oncol. 8, 1997, 1197-1206). EGFR has therefore emerged as an attractive target for the design and development of diagnostic and therapeutic agents that can specifically bind and inhibit the receptor's tyrosine kinase activity and signal transduction pathway in cancer cells. For example, the EGFR tyrosine kinase (EGFR-TK) reversible inhibitor TARCEVA® is approved by the FDA for treatment of NSCLC and advanced pancreatic cancer. Other anti-EGFR targeted molecules have also been approved, including Lapatinib and IRESSA®.

The epidermal growth factor receptor (EGFR) is one of the most investigated receptor protein tyrosine kinases and its link to non-small-cell lung cancer (NSCLC) is well established (A. Russo, T. et al., Oncotarget 2015, 6, 26814). However, over 75% of patients die five years after their NSCLC diagnosis. Tumors driven by activating mutations within the EGFR tyrosine kinase domain, e.g., point-mutation L858R or in-frame exon-19 deletions (ex19del) are initially sensitive to EGFR tyrosine kinase inhibitors (TKIs), such as gefitinib and erlotinib, (Paez, J. G., et al., Science (New York, N.Y.) 2004, 304, 1497; Lynch, T. J., et al., The New England Journal of Medicine 2004, 350, 2129), but these inhibitors are rendered resistant due to the acquisition of the secondary ‘gatekeeper’ T790M mutation (Pao, W., et al., PLoS Medicine 2005, 2, e73; Yu, H. A., et al., Clinical Cancer Research 2013, 19, 2240). Efforts to overcome first-generation TKI drug resistance resulted in the discovery and optimization of T790M-targeting irreversible inhibitors, which are rendered effective due to the ability to form covalent bonds with C797 (D. A. E. Cross, et al., Cancer Discovery 2014, 4, 1046; E. L. Kwak, et al., Proceedings of the National Academy of Sciences of the United States of America 2005, 102, 7665). Patients harboring T790M positive tumors respond well to treatment with AZD9291, and more recently this drug has been shown to be a superior treatment as a front-line therapy in untreated EGFR mutant NSCLC patients (J.-C. Soria, et al., The New England Journal of Medicine 2018, 378, 113). However, despite these successes, patients can acquire resistance to AZD9291 through the acquisition of the C797S mutation that precludes the ability for the drug to form their essential covalent bonds (K. S. Thress, et al., Nature Medicine 2015, 21, 560).

Thus, there is a need for potent small molecule EGFR inhibitors with alternative mechanisms of action targeting mutant EGFR.

### SUMMARY

In an aspect, provided herein is a compound of Formula I-H:

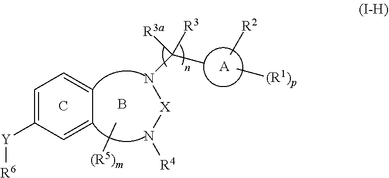

or a pharmaceutically acceptable salt thereof;
   wherein the variables are defined herein.

In an embodiment, the compound of Formula I-H is a compound of Formula Ia:

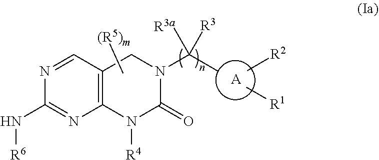

or a pharmaceutically acceptable salt thereof.

In another embodiment, the compound of Formula I-H is a compound of Formula Ib:

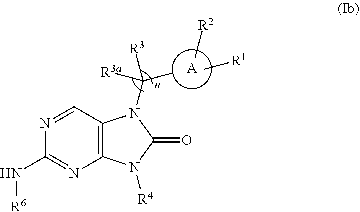

or a pharmaceutically acceptable salt thereof.

In another embodiment, the compound of Formula I-H is a compound of Formula Ic:

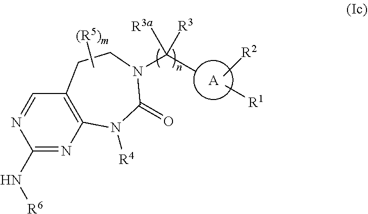

or a pharmaceutically acceptable salt thereof.

In yet another embodiment, the compound of Formula I-H is a compound of Formula Id:

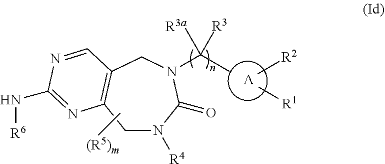

or a pharmaceutically acceptable salt thereof.

In still another embodiment, the compound of Formula I-H is a compound of Formula Ie:

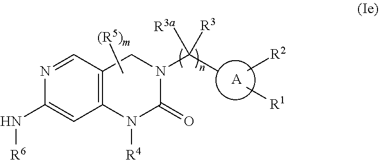

or a pharmaceutically acceptable salt thereof.

In another embodiment, the compound of Formula I-H is a compound of Formula If:

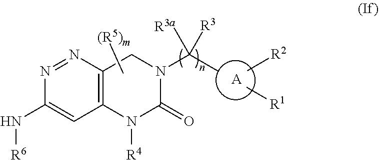

or a pharmaceutically acceptable salt thereof.

In an aspect, provided herein is a method of treating cancer or a proliferation disease, comprising administering to a subject in need thereof an effective amount of a compound disclosed herein or a pharmaceutical composition comprising a compound disclosed herein and a pharmaceutically acceptable carrier. In one embodiment, the cancer is lung cancer, breast cancer, glioma, squamous cell carcinoma, or prostate cancer. In another embodiment, the cancer is non-small cell lung cancer (NSCLC).

In another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

The disclosure also provides a kit comprising a compound capable of inhibiting EGFR activity selected from a compound of the present disclosure, or a pharmaceutically acceptable salt thereof, and instructions for use in treating cancer. In one embodiment, the kit further comprises components for performing a test to determine whether a subject has an activating mutation in EGFR or a resistance mutation in EGFR

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, including ±5%, 1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with wild-type or mutant EGFR an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the disclosure and a co-agent, are both administered to a patient simultaneously in the form of a single entity or dosage. The term “non-fixed combination” means that the active ingredients, e.g. a compound of the disclosure and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “EGFR” refers to epidermal growth factor receptor (alternately referred to as ErbB-1 or HER1) and may refer to the wild-type receptor or to a receptor containing one or more mutations.

As used herein, the term “HER” or Her” refers to members of the ErbB receptor tyrosine kinase family, including EGFR, ERBB2, HER3, and HER4.

As used herein, the term “allosteric site” refers to a site on EGFR other than the ATP binding site, such as that characterized in a crystal structure of EGFR. An “allosteric site” can be a site that is close to the ATP binding site, such as that characterized in a crystal structure of EGFR. For example, one allosteric site includes one or more of the following amino acid residues of epidermal growth factor receptor (EGFR): Lys745, Leu788, Ala743, Cys755, Leu777, Phe856, Asp855, Met766, Ile759, Glu762, and/or Ala763.

As used herein, the term “agent that prevents EGFR dimer formation,” or iterations thereof, refers to an agent that prevents dimer formation in which the C-lobe of the “activator” subunit impinges on the N-lobe of the “receiver” subunit. Examples of agents that prevent EGFR dimer formation include, but are not limited to, cetuximab, trastuzumab, panitumumab, and Mig6.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “haloalkyl” refers to an alkyl group, as defined above, substituted with one or more halo substituents, wherein alkyl and halo are as defined herein. Haloalkyl includes, by way of example, chloromethyl, trifluoromethyl, bromoethyl, chlorofluoroethyl, and the like.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. In an embodiment, “cycloalkyl” is C₃-C₁₀ cycloalkyl. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl.

As used herein, the term “bicyclic ring” means a fused ring system comprising two rings, wherein the first ring is aryl or heteroaryl and the second ring is cycloalkyl or heterocycloalkyl. The term “bicyclic ring” includes, but is not limited to, indoline, isoindoline-1,3-dione, isoindolin-1-one, and dihydro-indene. In an embodiment, the bicyclic ring is indoline.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. In an embodiment, “heterocyclyl” or “heterocycloalkyl” is 3-10 membered heterocycloalkyl. Heterocyclyl also includes polycyclic structures that may be bridged or spirocyclic in nature with each individual ring within the polycyclic structure varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydro-furanyl, tetrahydropyranyl (i.e., oxanyl), pyranyl, dioxanyl, aziridinyl, azetidinyl, pyrrolidinyl, 2,5-dihydro-1 H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo-[2.1.1]hexanyl, 6-azabicyclo[3.1.1] heptanyl, 2-azabicyclo[2.2.1]heptanyl, 3-azabicyclo[3.1.1]-heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-S-azabicyclo[3.1.1]-heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl.

As used herein, the term “heterocycloalkenyl” refers to monounsaturated or polyunsaturated monocyclic carbocycles containing at least one heteroatom selected from oxygen, sulfur, and nitrogen and may comprise benzo-fused analogues thereof. The term “heterocycloalkenyl” specifically includes, but is not limited to dihydropyranyl, dihydrothiopyranyl, dihydrothiophenyl, and tetrahydropyridinyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. In an embodiment, “aryl” is C₆-C₁₀ aryl. The term “aryl” includes, but is not limited to, phenyl, naphthyl, indanyl, and 1, 2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. In an embodiment, “heteroaryl” is 5-10 membered heteroaryl. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl, heteroaryl, cycloalkyl, bicyclic ring, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

Provided herein are compounds that are inhibitors of epidermal growth factor receptor (EGFR) useful in the treatment of kinase-mediated disorders, including cancer and other proliferation diseases. In an embodiment, the compounds provided herein are mutant selective EGFR inhibitors.

In an aspect, provided herein is a compound of Formula I-H:

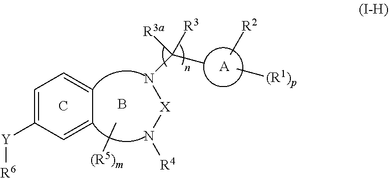

or a pharmaceutically acceptable salt thereof;
   wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, D, halo, CN, OR⁷, NO₂, C₁-C₆ alkyl, C₁-C₆ haloalkyl, C₁-C₆ alkyl-N(R⁷)₂, C₁-C₆ alkyl-OH, C(O)OH, ═O, N(R¹)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
p is 1 or 2;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein alkyl, cycloalkyl, heterocycloalkyl, aryl, and heteroaryl are each optionally substituted with R¹;
each R⁵ is independently selected from the group consisting of C₁-C₆ alkyl, ═O, halo, OR⁷, and N(R⁷)₂;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 8-10 membered bicyclic ring, and 3-10 membered heterocycloalkenyl all of which are optionally substituted 1, 2, 3, 4, or 5 times with R^{6a};
each R^{6a} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkyl, 3-10 membered heterocycloalkenyl, and 5-10 membered heteroaryl, wherein C₁-C₆ alkyl, OC₁-C₆ alkyl, N(C₁-C₆ alkyl)₂, heterocycloalkyl, heterocycloalkenyl, and heteroaryl are optionally substituted with halo, OH, C₁-C₆ alkyl, N(C₁-C₆ alkyl)₂, SO₂N(R⁷)₂, SO₂(C₁-C₆ alkyl), and 3-10 membered heterocycloalkyl, wherein 3-10 membered heterocycloalkyl is optionally substituted 1 or 2 times with R^{6aa};
R^{6aa} is selected from C₁-C₆ alkyl, C₁-C₆ haloalkyl, C₁-C₆ alkoxy, and C₁-C₆ alkyl-OH;
each R⁷ is independently selected from the group consisting of H, OH, halo, C₁-C₆ alkyl, C₁-C₆ alkyl-OH, C₆-C₁₀ aryl, 5-10 membered heteroaryl, C₃-C₁₀ cycloalkyl, and 3-10 membered heterocycloalkyl;
R⁸ is selected from OH, C(O)C₁-C₆ alkyl-OH, SO₂C₁-C₆ alkyl, SO₂C3-C₆ cycloalkyl, halo, C(O)C₁-C₆ alkyl, C₁-C₆ alkyl, NHC(O)C₁-C₆ alkyl, and C₁-C₆ alkoxy;
R² is selected from the group consisting of:

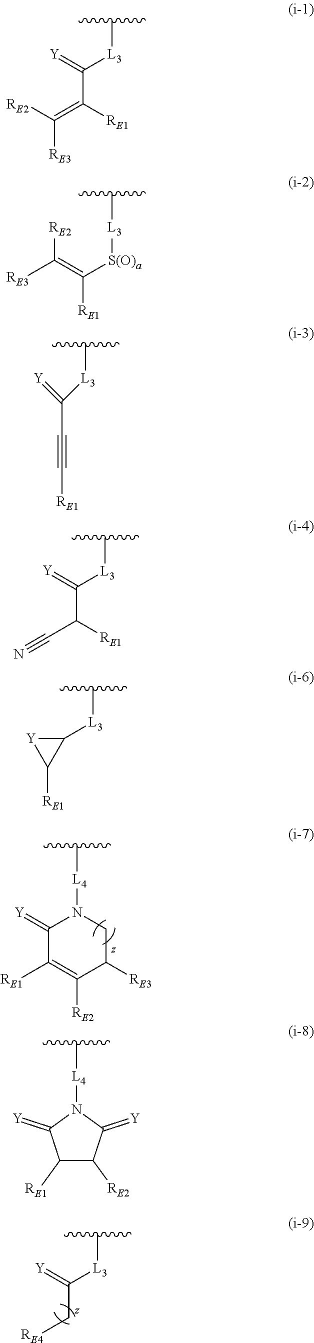

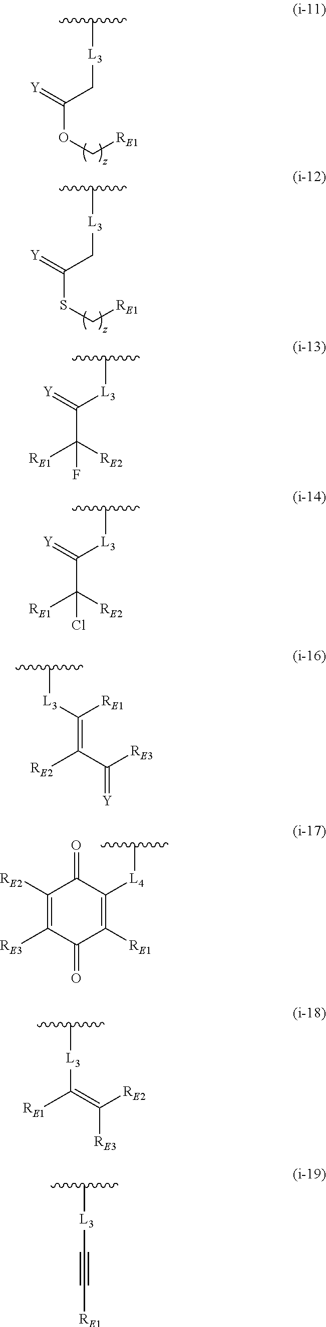

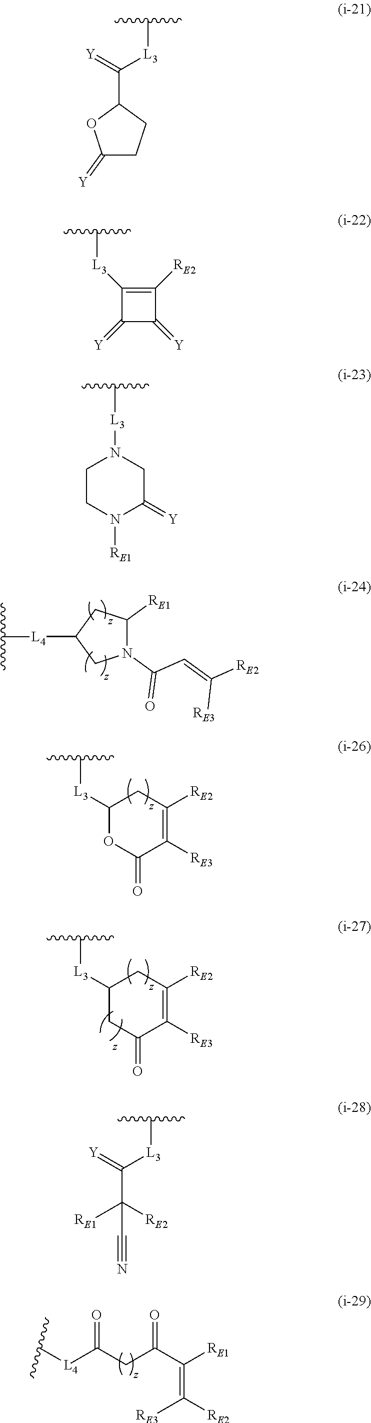

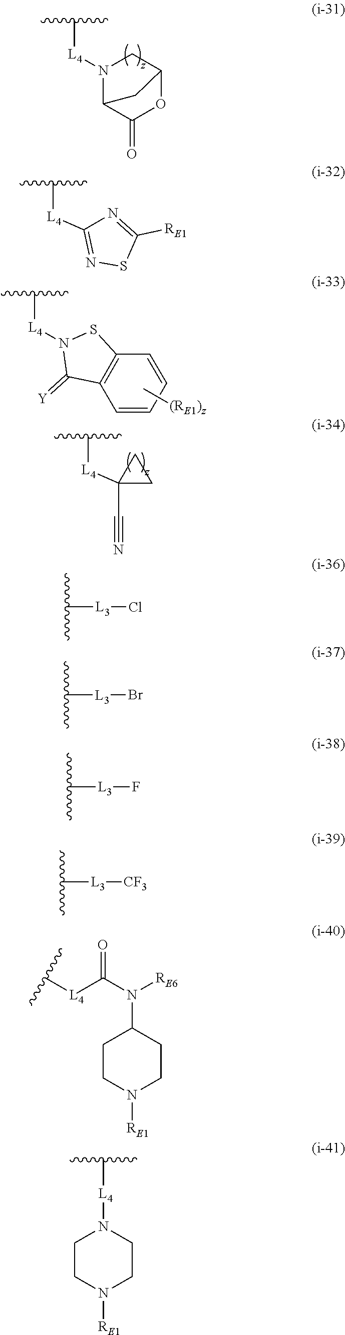

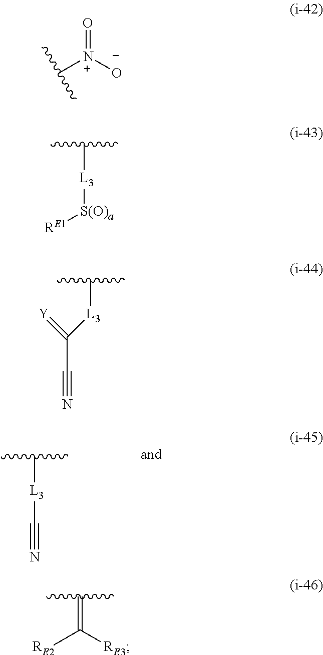

L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}CR_{L3b}—, cis-CR_{L3b}CR_{L3b}—, —C═C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a} —, or —NR_{L3a} S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹; L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C(O)OH, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{E}E groups, together with the atom to which they are attached, form 4-7 membered heterocycloalkyl;
R_{E6} is hydrogen, C₁-C₆ alkyl, or a nitrogen protecting group;
each Y is independently 0, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 0, 1, 2, or 3.

In an aspect, provided herein is a compound of Formula I-G:

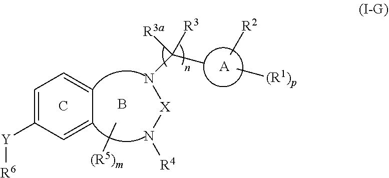

or a pharmaceutically acceptable salt thereof;
   wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, D, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
p is 1 or 2;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R¹;
each R⁵ is independently selected from the group consisting of C₁-C₆ alkyl, halo, OR⁷, and N(R⁷)₂;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 8-10 membered bicyclic ring, and 3-10 membered heterocycloalkenyl all of which are optionally substituted 1, 2, 3, 4, or 5 times with R^{6a};
each R^{6a} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkyl, 3-10 membered heterocycloalkenyl, and 5-10 membered heteroaryl, wherein C₁-C₆ alkyl, OC₁-C₆ alkyl, N(C₁-C₆ alkyl)₂, heterocycloalkyl, heterocycloalkenyl, and heteroaryl are optionally substituted with halo, OH, C₁-C₆ alkyl, N(C₁-C₆ alkyl)₂, SO₂N(R⁷)₂, and 3-10 membered heterocycloalkyl, wherein 3-10 membered heterocycloalkyl is optionally substituted 1 or 2 times with R^{6aa};
R^{6aa} is C₁-C₆ alkyl;
each R⁷ is independently selected from the group consisting of H, OH, halo, C₁-C₆ alkyl, C₁-C₆ alkyl-OH, C₆-C₁₀ aryl, 5-10 membered heteroaryl, C₃-C₁₀ cycloalkyl, and 3-10 membered heterocycloalkyl;
R⁸ is selected from C(O)C₁-C₆ alkyl-OH, SO₂C1-C₆ alkyl, SO₂C3-C₆ cycloalkyl, halo, C(O)C₁-C₆ alkyl, C₁-C₆ alkyl, NHC(O)C₁-C₆ alkyl, and C₁-C₆ alkoxy;
R² is selected from the group consisting of:

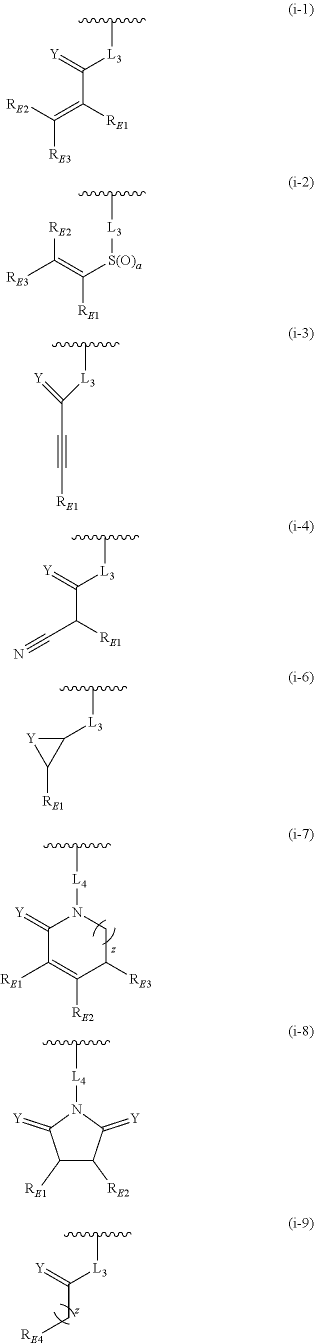

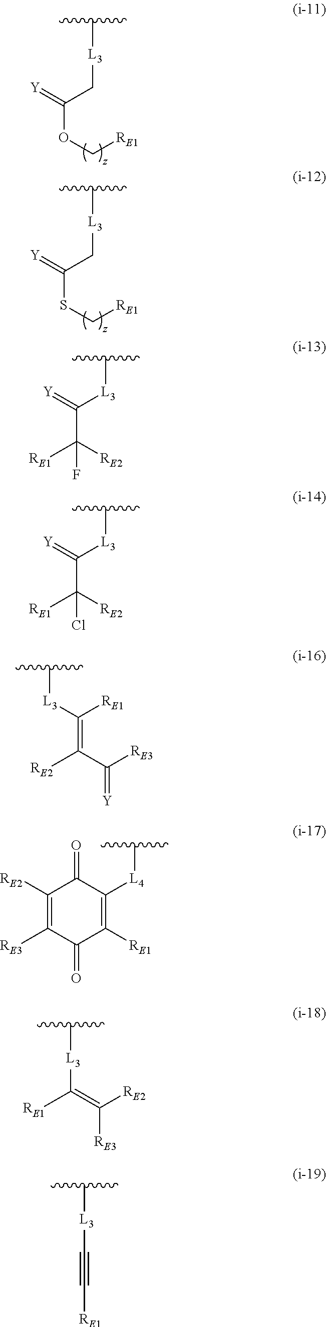

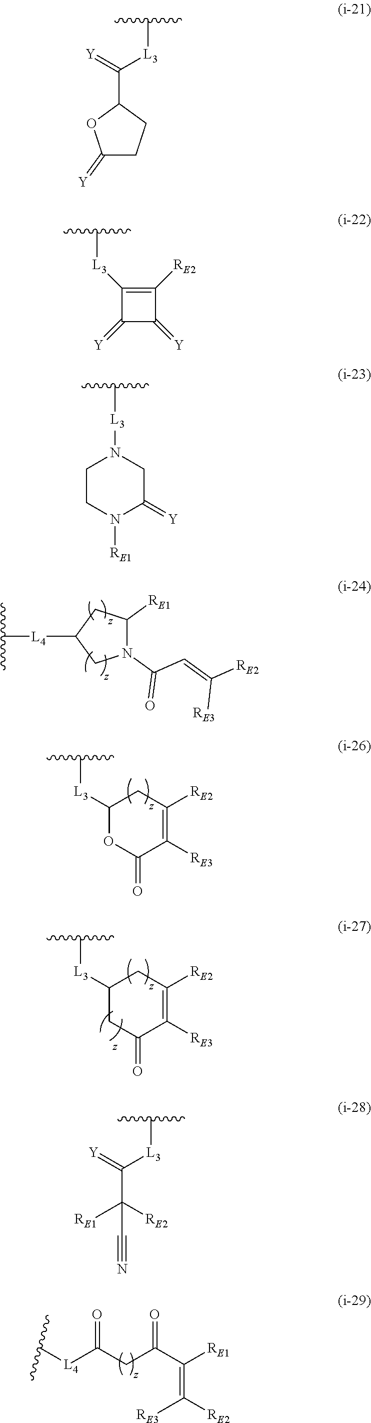

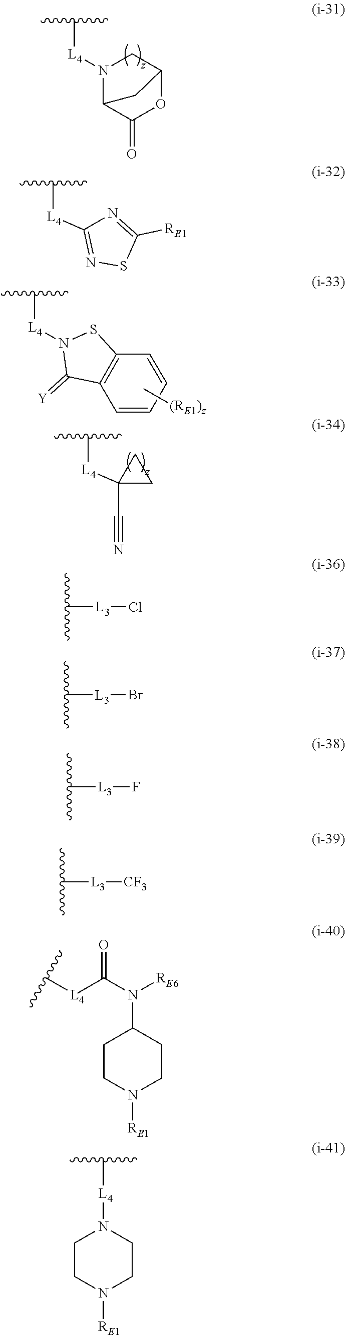

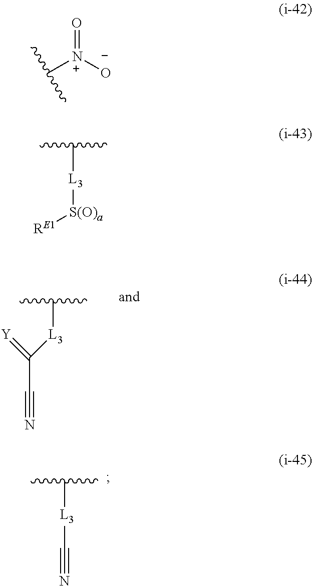

L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}, —SC(O)—, —C(O)S—, —OC(O)—, —C(O)O—, —NR_{L3a}C(S)—, —C(S)NR_{L3a}, trans-CR_{L3b}═CR_{L3b}—, cis-CR_{L3b}═CR_{L3b}—, —C≡C— —S(O)—, —S(O)O—, OS(O)—, —S(O)NR_{L3a}—, —NR_{L3a}S(O)—, —S(O)₂—, —S(O)₂O—, OS(O)₂—, —S(O)₂NR_{L3a}, or —NR_{L3a} S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{E}E groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

In an embodiment, Formula I-G is a compound of Formula I:

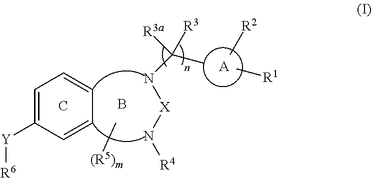

or a pharmaceutically acceptable salt thereof;
   wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R¹;
each R⁵ is independently selected from the group consisting of C₁-C₆ alkyl, halo, OR⁷, and N(R⁷)₂;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl all of which are substituted with 3-10 membered heterocycloalkyl, wherein the heterocycloalkyl is optionally substituted with C₁-C₆ alkyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
R⁸ is selected from C(O)C₁-C₆ alkyl-OH, SO₂C1-C₆ alkyl, SO₂C3-C₆ cycloalkyl, halo, C(O)C₁-C₆ alkyl, C₁-C₆ alkyl, and C₁-C₆ alkoxy;
R² is selected from the group consisting of:

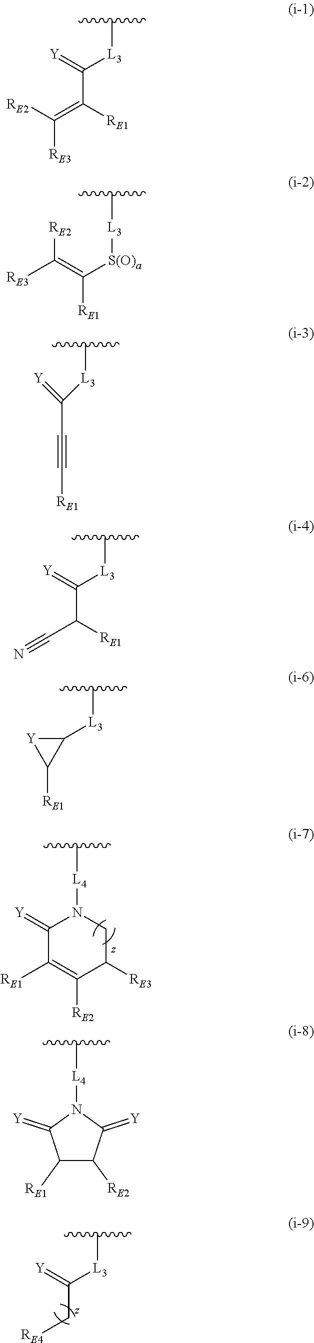

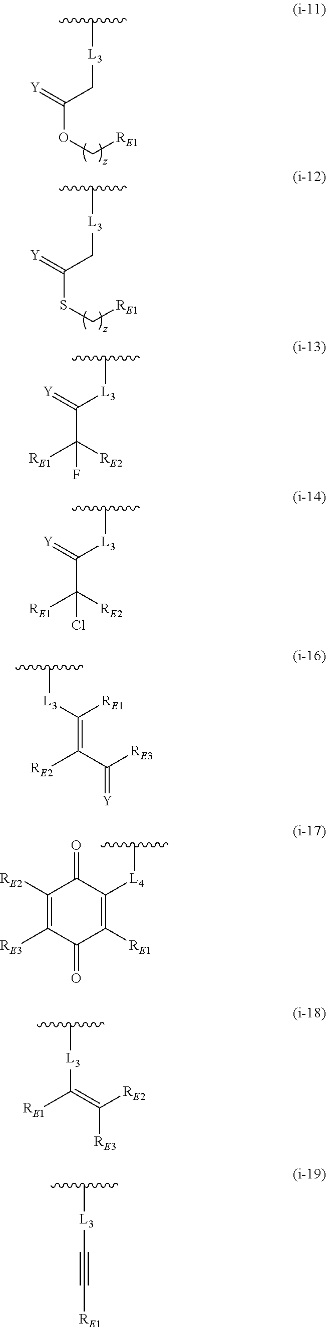

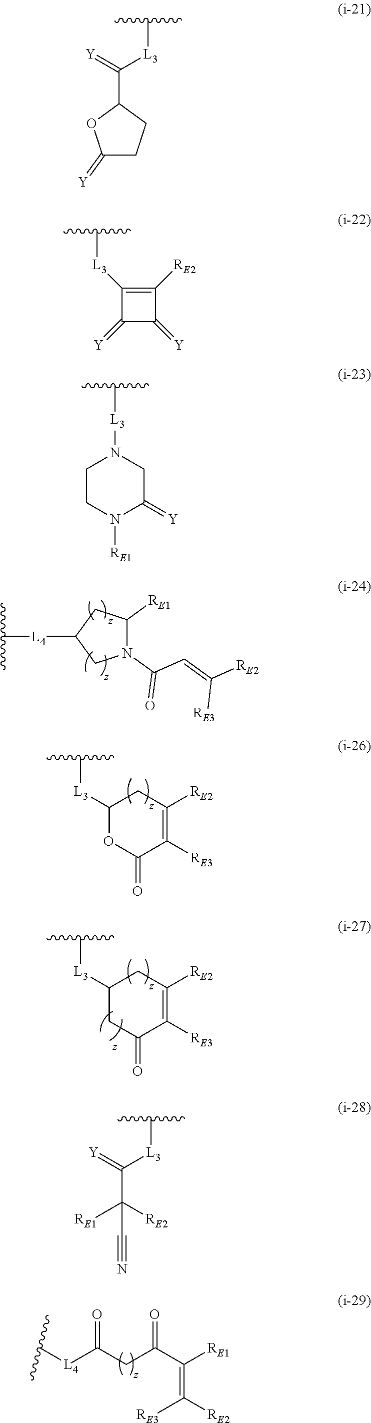

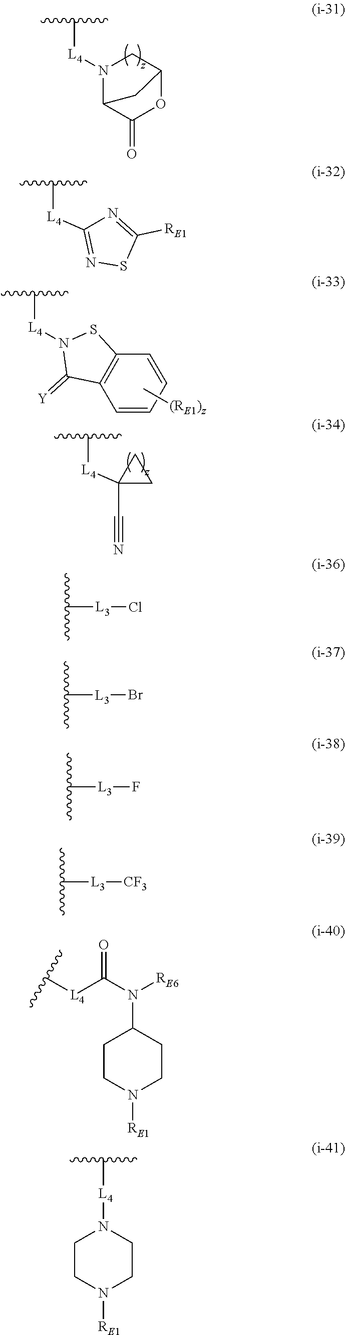

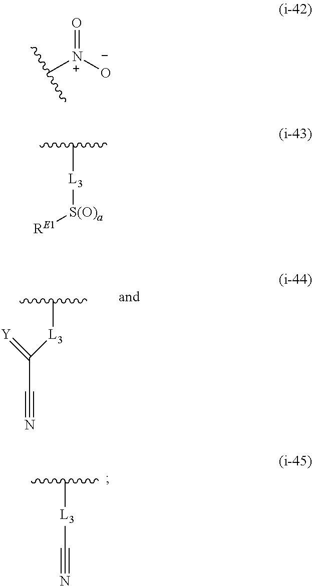

L₃ is a bond, —NH—, —N(C₁-C₄ alkyl)-, or C₁-C₄ alkylene, optionally wherein one or more carbons is independently replaced with —C(O)—, —O—, —S—, —NR_{L3a}——, —NR_{L3a}C(O)—, —C(O)NR_{L3a}, —SC(O)—, —C(O)S—, —OC(O)—, —C(O)O—, —NR_{L3a}C(S)—, —C(S)NR_{L3a}, trans-CR_{L3b}═CR_{L3b}—, cis-CR_{L3b}═CR_{L3b}—, —C═C—, —S(O)—, —S(O)O—, OS(O)—, —S(O)NR_{L3a}, —NR_{L3a}S(O)—, —S(O)₂—, —S(O)₂O—, OS(O)₂—, —S(O)₂NR_{L3a}, or —NR_{L3a} S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

In an embodiment of Formula I-G,
p is 2;
R⁶ is 8-10 membered bicyclic ring optionally substituted 1, 2, 3, 4, or 5 times with R^{6a};
each R^{6a} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkenyl, and 5-10 membered heteroaryl, wherein C₁-C₆ alkyl, OC₁-C₆ alkyl, N(C₁-C₆ alkyl)₂, heterocycloalkenyl, and heteroaryl are optionally substituted with halo, OH, N(C₁-C₆ alkyl)₂, SO₂N(R⁷)₂, 3-10 membered heterocycloalkyl, wherein 3-10 membered heterocycloalkyl is optionally substituted 1 or 2 times with R^{6aa};
R^{6aa} is C₁-C₆ alkyl;
each R⁷ is independently selected from H and C₁-C₆ alkyl-OH; and
R⁸ is NHC(O)C₁-C₆ alkyl.

In an embodiment of Formula I,
X is C(O);
Y is NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, and 6-10 membered bicyclic ring;
B is 5-7 membered heterocyclic ring;
C is pyrimidine, pyridine, or pyridazine;
R¹ is selected from the group consisting of H, halo, CN, C₁-C₆ alkyl, C₁-C₆ haloalkyl, O—C₁-C₆ alkyl, 0(3-10 membered heterocycloalkyl), NHSO₂C1-C₆ alkyl, and NH(3-10 membered heterocycloalkyl);
R³ and R^{3a} are each independently selected from the group consisting of H and C₁-C₆ alkyl;
alternatively, R³ and R^{3a} optionally combine to form C₃-C₁₀ cycloalkyl;
n is 0, 1, or 2;
R⁴ is C₁-C₆ alkyl or 3-10 membered heterocycloalkyl optionally substituted with R¹;
each R⁵ is independently selected from the group consisting of C₁-C₆ alkyl and halo;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl and 5-10 membered 30 heteroaryl, both of which are substituted with 3-10 membered heterocycloalkyl optionally substituted with C₁-C₆ alkyl;
R⁸ is C(O)C₁-C₆ alkyl-OH or SO₂C₃-C₆ cycloalkyl;
R² is selected from the group consisting of:

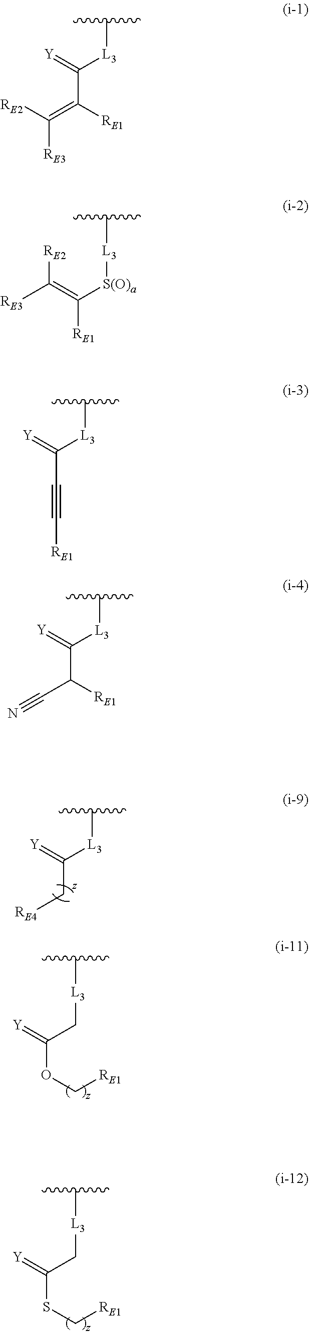

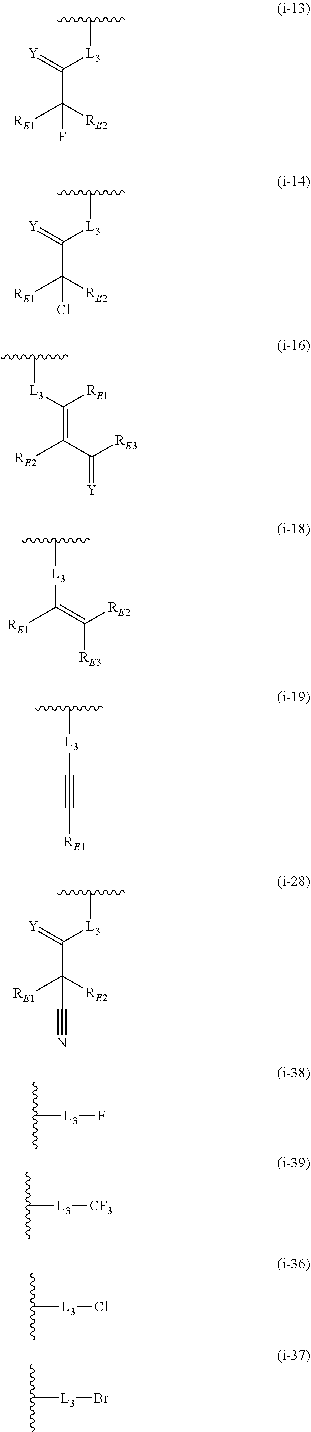

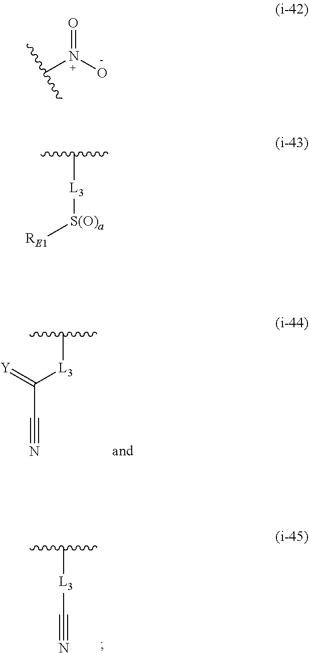

L₃ is a bond, —NH—, —N(C₁-C₄ alkyl)-, or C₁-C₄ alkylene;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

In another embodiment of Formula I,
X is C(O);
Y is NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, and 6-10 membered bicyclic ring;
B is 5-7 membered heterocyclic ring;
C is pyrimidine, pyridine, or pyridazine;
R¹ is selected from the group consisting of H, halo, CN, C₁-C₆ alkyl, C₁-C₆ haloalkyl, O—C₁-C₆ alkyl, 0(3-10 membered heterocycloalkyl), NHSO₂C₁-C₆ alkyl, and NH(3-10 membered heterocycloalkyl);
R³ and R^{3a} are each independently selected from the group consisting of H and C₁-C₆ alkyl;
alternatively, R³ and R^{3a} optionally combine to form C₃-C₁₀ cycloalkyl;
n is 0, 1, or 2;
R⁴ is C₁-C₆ alkyl or 3-10 membered heterocycloalkyl optionally substituted with R¹;
each R⁵ is independently selected from the group consisting of C₁-C₆ alkyl and halo;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl and 5-10 membered heteroaryl, both of which are substituted with 3-10 membered heterocycloalkyl optionally substituted with C₁-C₆ alkyl;
R⁸ is C(O)C₁-C₆ alkyl-OH or SO₂C₃-C₆ cycloalkyl; and
R² is

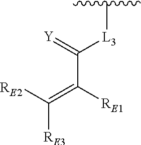

wherein
L³ is a bond, N(H), —N(C₁-C₄ alkyl)-, or C₁-C₄ alkylene;
Y is O, S, or CH₂; and
each of R_{E1}, R_{E2}, and R_{E3}, are independently selected from H, halo, and C₁-C₆ alkyl.

In another embodiment, the compound of Formula I is a compound of Formula Ia:

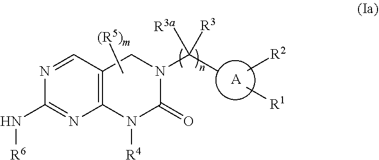

or a pharmaceutically acceptable salt thereof.

In yet another embodiment, the compound of Formula I is a compound of Formula Ib:

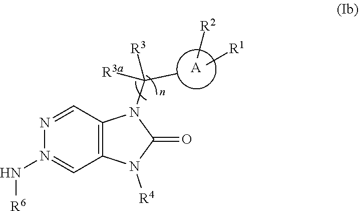

or a pharmaceutically acceptable salt thereof.

In still another embodiment, the compound of Formula I is a compound of Formula Ic:

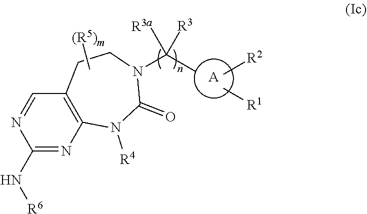

or a pharmaceutically acceptable salt thereof.

In an embodiment, the compound of Formula I is a compound of Formula Id:

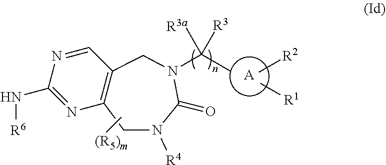

or a pharmaceutically acceptable salt thereof.

In another embodiment, the compound of Formula I is a compound of Formula Ie:

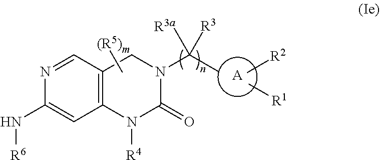

or a pharmaceutically acceptable salt thereof.

In yet another embodiment, the compound of Formula I is a compound of Formula If:

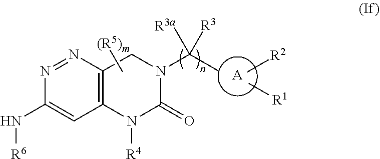

or a pharmaceutically acceptable salt thereof.

In still another embodiment, X is C(O), C(S), or SO₂. In an embodiment, X is C(O). In another embodiment, X is C(S). In yet another embodiment, X is C(NH). In still another embodiment, X is SO₂.

In an embodiment, Y is NH. In another embodiment, Y is O. In yet another embodiment, Y is S.

In still another embodiment, A is selected from the group consisting of C₂-C₆ alkenyl, C₆-C₁₀ aryl, 5-10 membered heteroaryl, 3-10 membered heterocycloalkyl, and 6-10 membered bicyclic ring.

In an embodiment, A is 4-7 membered heterocycloalkyl. In another embodiment, A is 7 membered heterocycloalkyl. In yet another embodiment, A is spiro 7 membered heterocycloalkyl. In still another embodiment, A is 4 membered heterocycloalkyl. In an embodiment A is 5 membered heterocycloalkyl.

In an embodiment, A is selected from the group consisting of phenyl, 5-6 membered heteroaryl, 5-6 membered heterocycloalkyl, and 9-10 membered bicyclic ring. In another embodiment, A is selected from the group consisting of phenyl, thiophene, pyrrole, pyridine, pyrrolidine, piperidine, indoline, and tetrahydroquinoline. In an embodiment, A is C₂-C₆ alkenyl.

In an embodiment, B is selected from the group consisting of

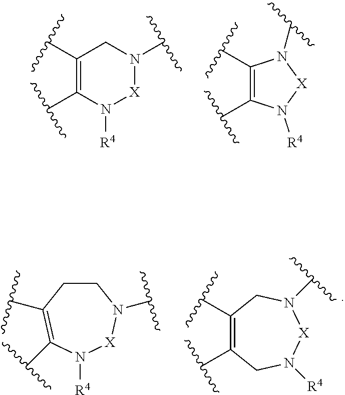

In another embodiment, B is

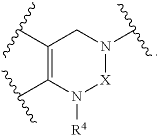

In yet another embodiment, C is pyrimidine. In still another embodiment, C is pyridine. In an embodiment, C is pyridazine.

In another embodiment, R¹ is selected from the group consisting of H, halo, CN, C₁-C₃ alkyl, C₁-C₃ haloalkyl, 0-C₁-C₃ alkyl, 0(3-4 membered heterocycle), NHSO₂C₁-C₃ alkyl, and NH(3-5 membered heterocycloalkyl). In an embodiment, R¹ is selected from the group consisting of H, D, OH, C₁-C₃ alkyl, C₁-C₃ haloalkyl, and O—C₁-C₃ alkyl. In an embodiment, R¹ is C₆-C₁₀ aryl. In an embodiment, R¹ is phenyl.

In yet another embodiment,
R³ and R^{3a} are each independently H or C₁-C₃ alkyl;
alternatively, R³ and R^{3a} optionally combine to form C₃-C₅ cycloalkyl.

In still another embodiment, n is 0. In an embodiment, n is 1. In another embodiment, n is 2.

In yet another embodiment, R⁴ is C₁-C₃ alkyl or 3-6 membered heterocycloalkyl optionally substituted with R⁸. In still another embodiment, R⁴ is C₁-C₃ alkyl, pyrrolidine, or piperidine, wherein pyrrolidine and piperidine are optionally substituted with R⁸. In an embodiment, R⁴ is 4-7 membered cycloalkyl or 5-7 membered heterocycloalkyl.

In an embodiment, R⁵ is C₁-C₃ alkyl or halo.

In another embodiment, R⁴ and R⁵ combine to form 3-10 membered heterocycloalkyl.

In still another embodiment, R⁴ and R⁵ combine with B to form heterobicyclo[2.2.1]heptane or heterobicyclo[3.2.1]octane.

In an embodiment, two R⁵ combine to form C₃-C₁₀ cycloalkyl. In another embodiment, two R⁵ combine to form C₃-C₆ cycloalkyl.

In still another embodiment, m is 0. In an embodiment, m is 1. In another embodiment, m is 2.

In yet another embodiment, R⁶ is selected from the group consisting of phenyl and 5-6 membered heteroaryl, both of which are substituted with 5-6 membered heterocycloalkyl optionally substituted with C₁-C₃ alkyl.

In an embodiment, R⁶ is selected from the group consisting of phenyl, 5-6 membered heteroaryl, and 5-6 membered heterocycloalkenyl all of which are optionally substituted 1, 2, or 3 times with R^{6a}, and wherein each R^{6a} is independently selected from the group consisting of halo, CN, C₁-C₃ alkyl, OC₁-C₃ alkyl, N(C₁-C₃ alkyl)₂, SO₂N(H)(C₁-C₃ alkyl-OH), 6-8 membered heterocycloalkyl, 5-6 membered heterocycloalkenyl, and 5-6 membered heteroaryl, wherein C₁-C₃ alkyl, OC₁-C₃ alkyl, 6-8 membered heterocycloalkyl, 5-6 membered heterocycloalkenyl, and 5-6 membered heteroaryl are optionally substituted with halo, OH, C₁-C₃ alkyl, N(C₁-C₃ alkyl)₂, SO₂N(H)(C₁-C₃ alkyl), and 5-6 membered heterocycloalkyl, wherein 5-6 membered heterocycloalkyl is optionally substituted with C₁-C₃ alkyl.

In an embodiment, R⁶ is phenyl, R^{6a} is piperidinyl, and R^{6aa} is C₁-C₃ alkyl. In an embodiment, R⁶ is phenyl, R^{6a} is piperazinyl, and R^{6aa} is C₁-C₃ alkyl. In another embodiment R⁶ is phenyl and R^{6a} is OC₁-C₃ alkyl-5 membered heterocycloalkyl. In yet another embodiment, R⁶ is 5 membered heteroaryl and R^{6a} is C₁-C₃ alkyl-OH.

In another embodiment, R^{6a} is 6-8 membered heterocycloalkyl and has the following structure:

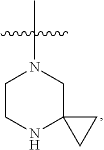 [SMILES: *N1CCNC2(CC2)C1]

wherein said heterocycloalkyl is optionally substituted with C₁-C₃ alkyl.

In still another embodiment, R⁵ is C(O)C₁-C₃ alkyl-OH or SO₂C₃-C₄ cycloalkyl. In an embodiment, R⁸ is OH.

In an embodiment, R² is

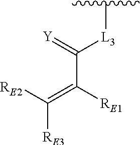

wherein
L³ is a bond, N(H), —N(C₁-C₄ alkyl)-, or C₁-C₄ alkylene;
Y is O, S, or CH₂; and
each of R_{E1}, R_{E2}, and R_{E3}, are independently selected from H, halo, and C₁-C₆ alkyl.

In another embodiment, R² is selected from the group consisting of

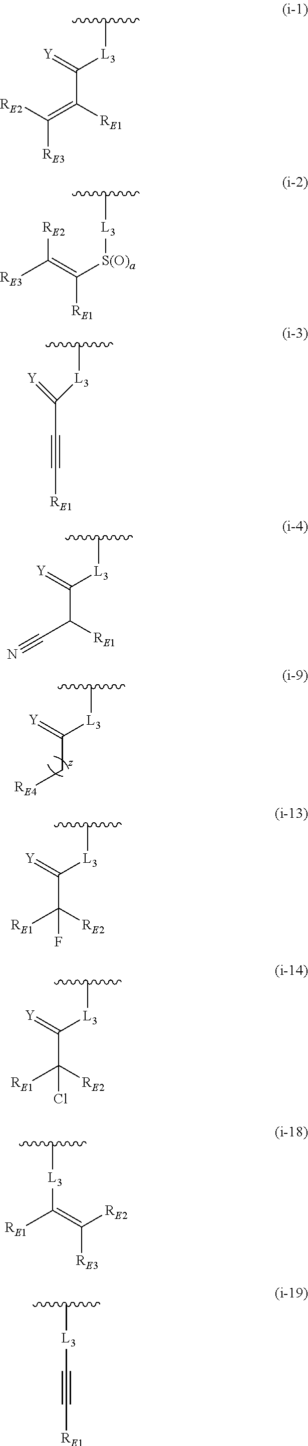

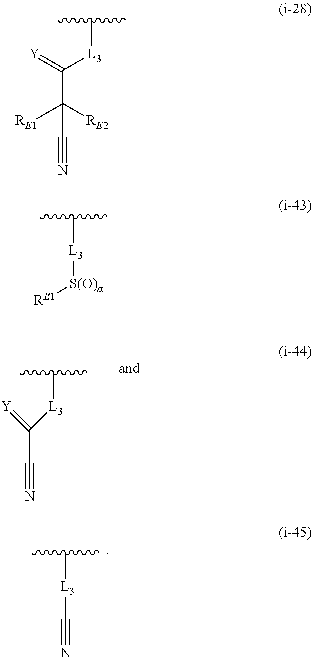

In yet another embodiment, R² is selected from the group consisting of

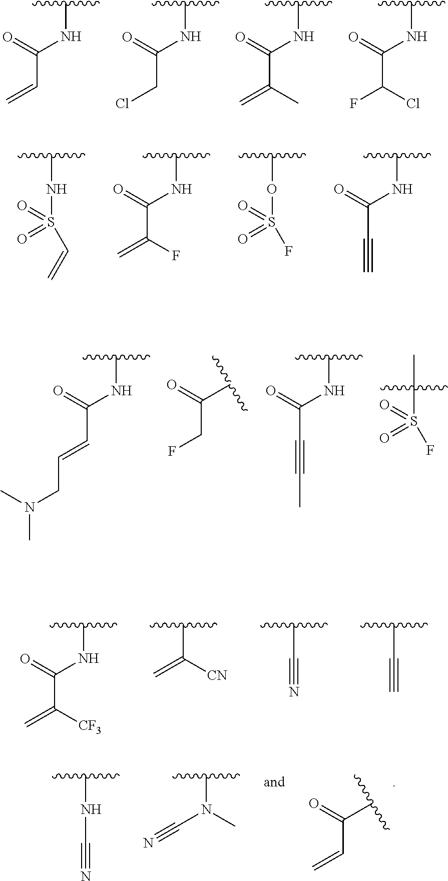

In still another embodiment, R² is selected from the group consisting of

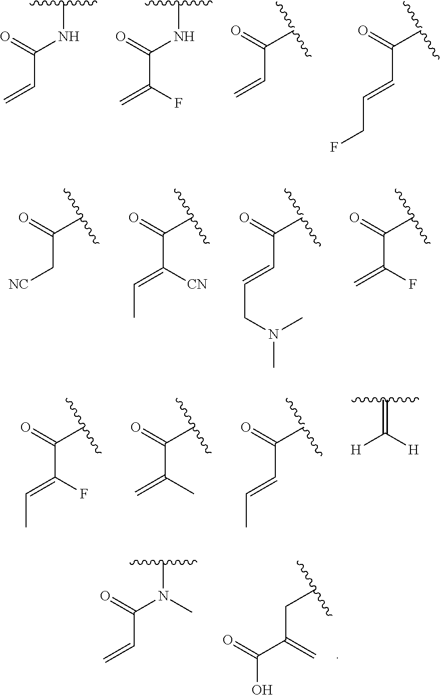

In another embodiment, R² is selected from the group consisting of

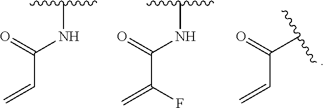

In yet another embodiment, R² is

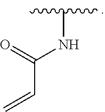 [SMILES: *NC(=O)C=C]

In an embodiment, R² is selected from the group consisting of

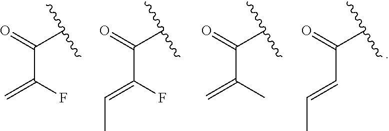

In another embodiment, R² is

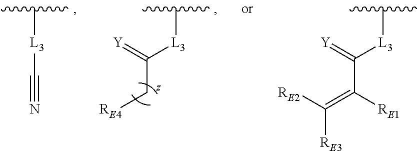

wherein
L³ is a bond;
Y is O;
z is 1;
each of R_{E1}, R_{E2}, R_{E3}, and R_{E4} are independently selected from CN and C₁-C₆ alkyl
wherein C₁-C₆ alkyl is optionally substituted with one R⁹; and
R⁹ is selected from the group consisting of halo and N(C₁-C₆ alkyl)₂.

In still another embodiment, R² is selected from the group consisting of

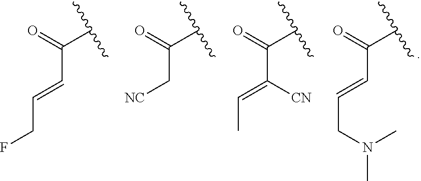

In an embodiment, the compound is selected from the group consisting of a compound in Table 1.

TABLE 1

| Compound No. | Structure |
|---|---|
| 001 | 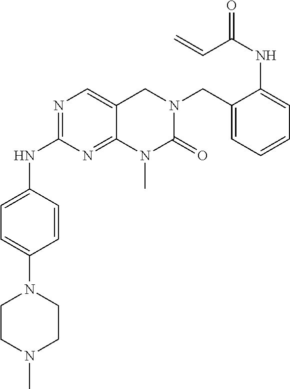 [SMILES: C=CC(=O)Nc1ccccc1CN1Cc2cnc(Nc3ccc(N4CCN(C)CC4)cc3)nc2N(C)C1=O] |
| 002 | 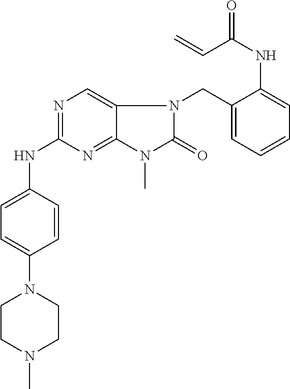 [SMILES: C=CC(=O)Nc1ccccc1Cn1c(=O)n(C)c2nc(Nc3ccc(N4CCN(C)CC4)cc3)ncc21] |
| 003 | 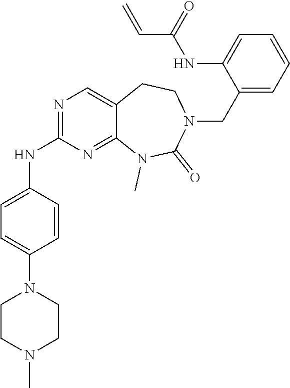 [SMILES: C=CC(=O)Nc1ccccc1CN1CCc2cnc(Nc3ccc(N4CCN(C)CC4)cc3)nc2N(C)C1=O] |
| 004 | 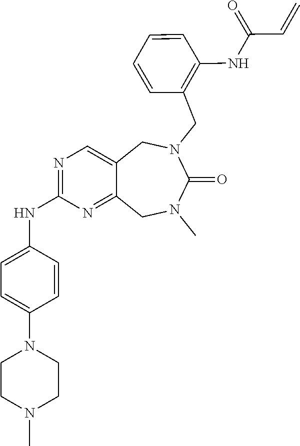 [SMILES: C=CC(=O)Nc1ccccc1CN1Cc2cnc(Nc3ccc(N4CCN(C)CC4)cc3)nc2CN(C)C1=O] |
| 005 | 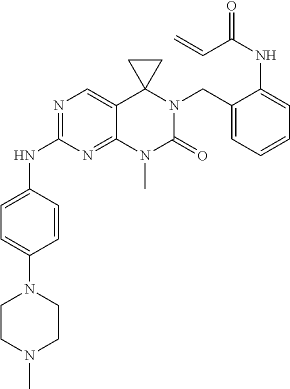 [SMILES: C=CC(=O)Nc1ccccc1CN1C(=O)N(C)c2nc(Nc3ccc(N4CCN(C)CC4)cc3)ncc2C12CC2] |
| 006 | 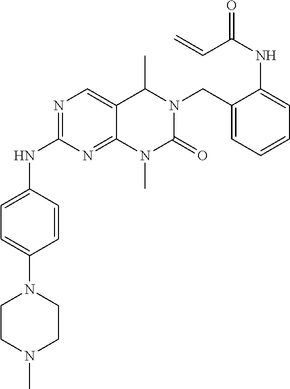 [SMILES: C=CC(=O)Nc1ccccc1CN1C(=O)N(C)c2nc(Nc3ccc(N4CCN(C)CC4)cc3)ncc2C1C] |
| 007 | 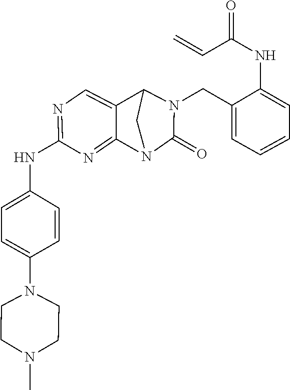 [SMILES: C=CC(=O)Nc1ccccc1CN1C(=O)N2CC1c1cnc(Nc3ccc(N4CCN(C)CC4)cc3)nc12] |
| 008 | 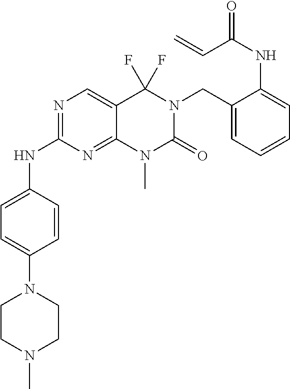 [SMILES: C=CC(=O)Nc1ccccc1CN1C(=O)N(C)c2nc(Nc3ccc(N4CCN(C)CC4)cc3)ncc2C1(F)F] |
| 009 | 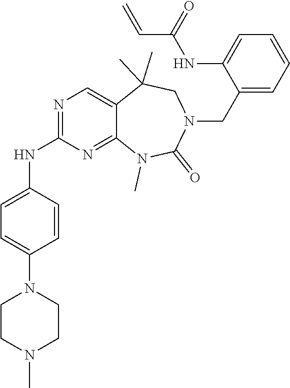 [SMILES: C=CC(=O)Nc1ccccc1CN1CC(C)(C)c2cnc(Nc3ccc(N4CCN(C)CC4)cc3)nc2N(C)C1=O] |
| 010 | 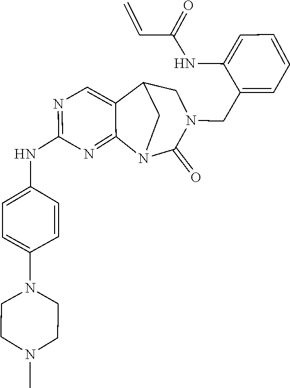 [SMILES: C=CC(=O)Nc1ccccc1CN1CC2CN(C1=O)c1nc(Nc3ccc(N4CCN(C)CC4)cc3)ncc12] |
| 011 | 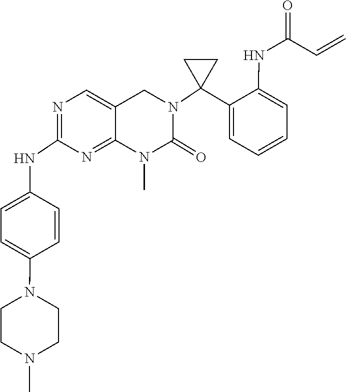 [SMILES: C=CC(=O)Nc1ccccc1C1(N2Cc3cnc(Nc4ccc(N5CCN(C)CC5)cc4)nc3N(C)C2=O)CC1] |
| 012 | 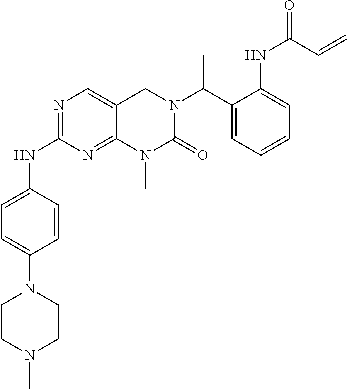 [SMILES: C=CC(=O)Nc1ccccc1C(C)N1Cc2cnc(Nc3ccc(N4CCN(C)CC4)cc3)nc2N(C)C1=O] |
| 013 | 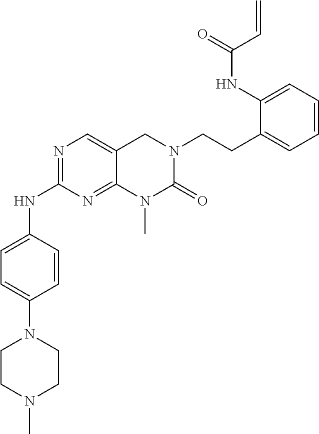 [SMILES: C=CC(=O)Nc1ccccc1CCN1Cc2cnc(Nc3ccc(N4CCN(C)CC4)cc3)nc2N(C)C1=O] |
| 014 | 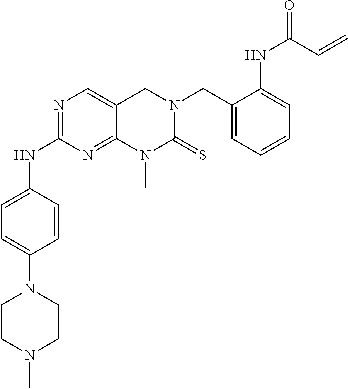 [SMILES: C=CC(=O)Nc1ccccc1CN1Cc2cnc(Nc3ccc(N4CCN(C)CC4)cc3)nc2N(C)C1=S] |
| 015 | 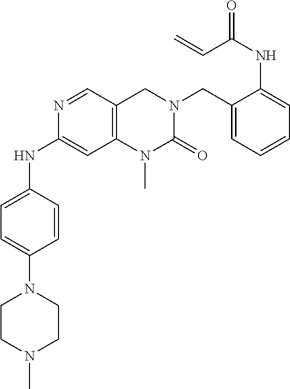 [SMILES: C=CC(=O)Nc1ccccc1CN1Cc2cnc(Nc3ccc(N4CCN(C)CC4)cc3)cc2N(C)C1=O] |
| 016 | 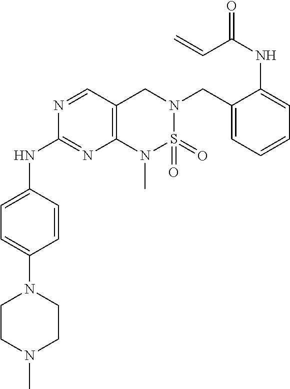 [SMILES: C=CC(=O)Nc1ccccc1CN1Cc2cnc(Nc3ccc(N4CCN(C)CC4)cc3)nc2N(C)S1(=O)=O] |
| 017 | 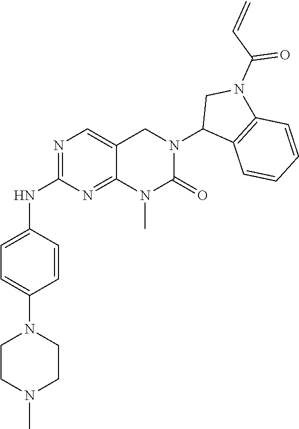 [SMILES: C=CC(=O)N1CC(N2Cc3cnc(Nc4ccc(N5CCN(C)CC5)cc4)nc3N(C)C2=O)c2ccccc21] |
| 018 | 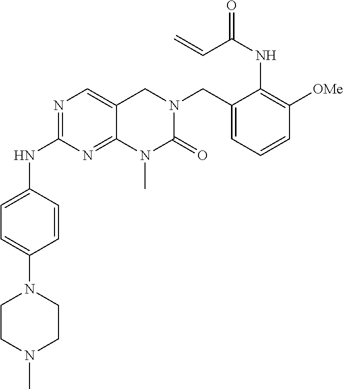 [SMILES: C=CC(=O)Nc1c(CN2Cc3cnc(Nc4ccc(N5CCN(C)CC5)cc4)nc3N(C)C2=O)cccc1OC] |
| 019 | 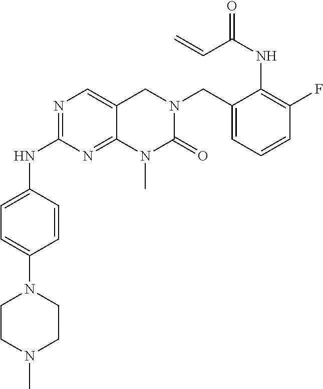 [SMILES: C=CC(=O)Nc1c(F)cccc1CN1Cc2cnc(Nc3ccc(N4CCN(C)CC4)cc3)nc2N(C)C1=O] |
| 020 | 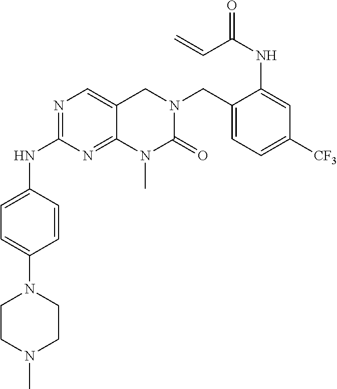 [SMILES: C=CC(=O)Nc1cc(C(F)(F)F)ccc1CN1Cc2cnc(Nc3ccc(N4CCN(C)CC4)cc3)nc2N(C)C1=O] |
| 021 | 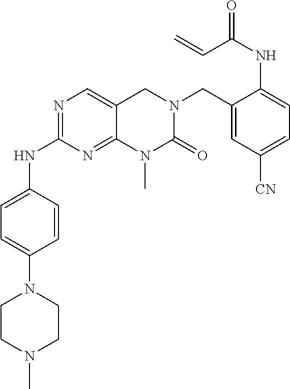 [SMILES: C=CC(=O)Nc1ccc(C#N)cc1CN1Cc2cnc(Nc3ccc(N4CCN(C)CC4)cc3)nc2N(C)C1=O] |
| 022 | 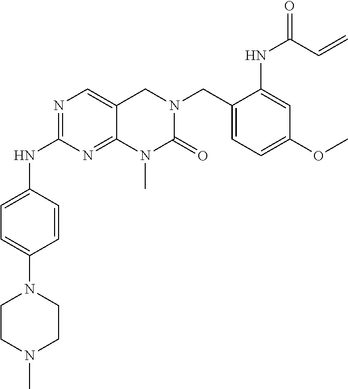 [SMILES: C=CC(=O)Nc1cc(OC)ccc1CN1Cc2cnc(Nc3ccc(N4CCN(C)CC4)cc3)nc2N(C)C1=O] |
| 023 | 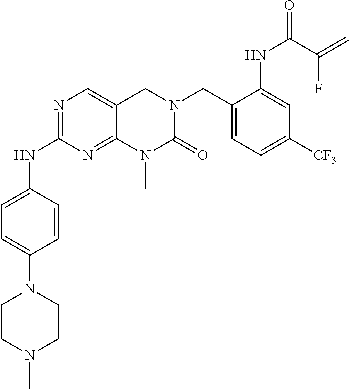 [SMILES: C=C(F)C(=O)Nc1cc(C(F)(F)F)ccc1CN1Cc2cnc(Nc3ccc(N4CCN(C)CC4)cc3)nc2N(C)C1=O] |
| 024 | 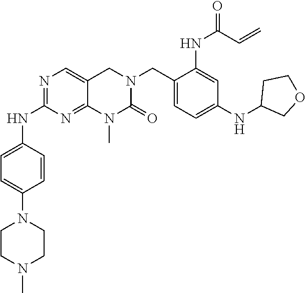 [SMILES: C=CC(=O)Nc1cc(NC2CCOC2)ccc1CN1Cc2cnc(Nc3ccc(N4CCN(C)CC4)cc3)nc2N(C)C1=O] |
| 025 | 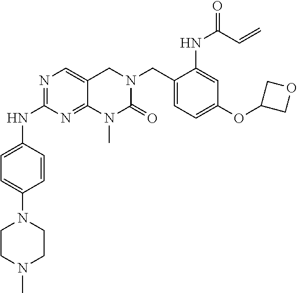 [SMILES: C=CC(=O)Nc1cc(OC2COC2)ccc1CN1Cc2cnc(Nc3ccc(N4CCN(C)CC4)cc3)nc2N(C)C1=O] |
| 026 | 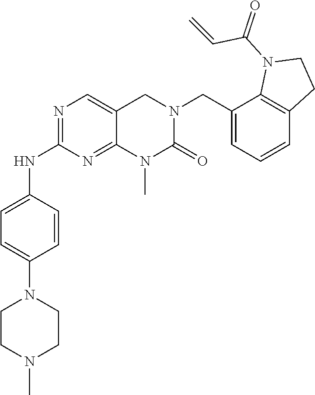 [SMILES: C=CC(=O)N1CCc2cccc(CN3Cc4cnc(Nc5ccc(N6CCN(C)CC6)cc5)nc4N(C)C3=O)c21] |
| 027 | 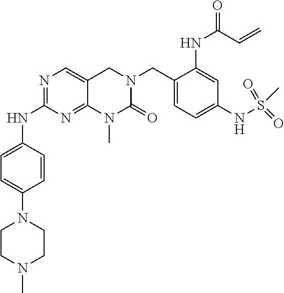 [SMILES: C=CC(=O)Nc1cc(NS(C)(=O)=O)ccc1CN1Cc2cnc(Nc3ccc(N4CCN(C)CC4)cc3)nc2N(C)C1=O] |
| 028 | 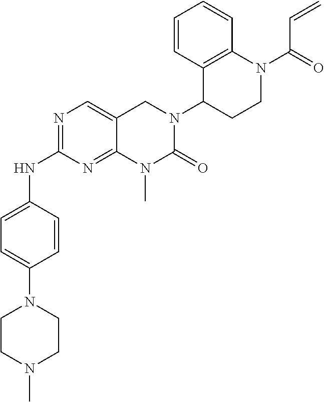 [SMILES: C=CC(=O)N1CCC(N2Cc3cnc(Nc4ccc(N5CCN(C)CC5)cc4)nc3N(C)C2=O)c2ccccc21] |
| 029 | 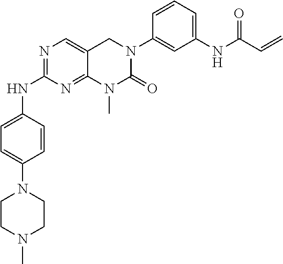 [SMILES: C=CC(=O)Nc1cccc(N2Cc3cnc(Nc4ccc(N5CCN(C)CC5)cc4)nc3N(C)C2=O)c1] |
| 030 | 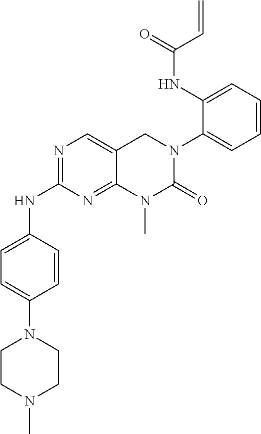 [SMILES: C=CC(=O)Nc1ccccc1N1Cc2cnc(Nc3ccc(N4CCN(C)CC4)cc3)nc2N(C)C1=O] |
| 031 | 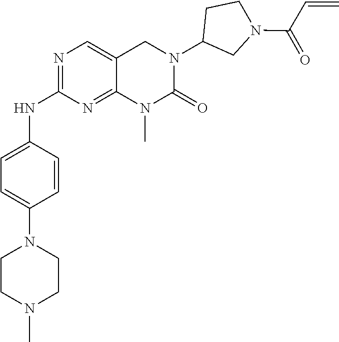 [SMILES: C=CC(=O)N1CCC(N2Cc3cnc(Nc4ccc(N5CCN(C)CC5)cc4)nc3N(C)C2=O)C1] |
| 032 | 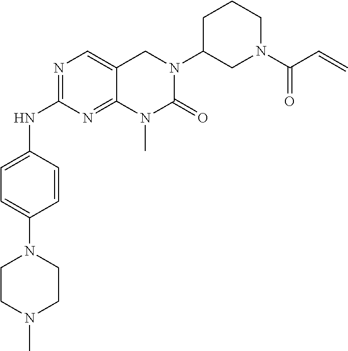 [SMILES: C=CC(=O)N1CCCC(N2Cc3cnc(Nc4ccc(N5CCN(C)CC5)cc4)nc3N(C)C2=O)C1] |
| 033 | 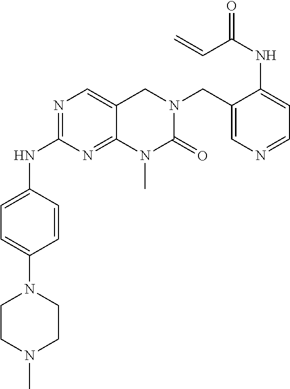 [SMILES: C=CC(=O)Nc1ccncc1CN1Cc2cnc(Nc3ccc(N4CCN(C)CC4)cc3)nc2N(C)C1=O] |
| 034 | 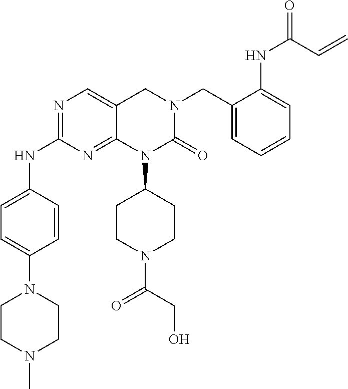 |
| 035 | 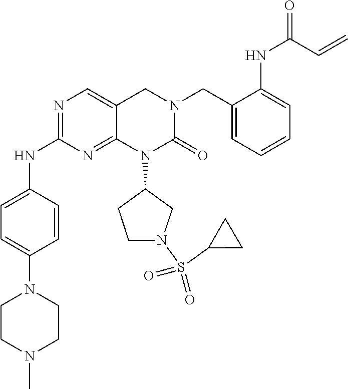 [SMILES: C=CC(=O)Nc1ccccc1CN1Cc2cnc(Nc3ccc(N4CCN(C)CC4)cc3)nc2N([C@H]2CCN(S(=O)(=O)C3CC3)C2)C1=O] |
| 036 | 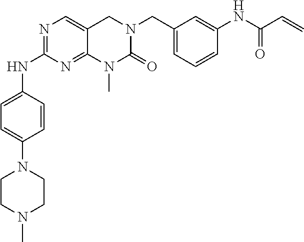 [SMILES: C=CC(=O)Nc1cccc(CN2Cc3cnc(Nc4ccc(N5CCN(C)CC5)cc4)nc3N(C)C2=O)c1] |
| 037 | 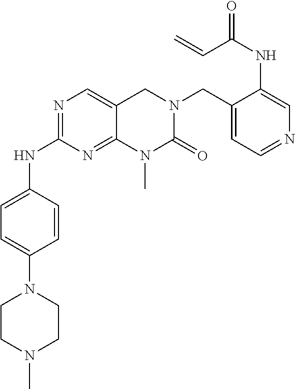 [SMILES: C=CC(=O)Nc1cnccc1CN1Cc2cnc(Nc3ccc(N4CCN(C)CC4)cc3)nc2N(C)C1=O] |
| 038 | 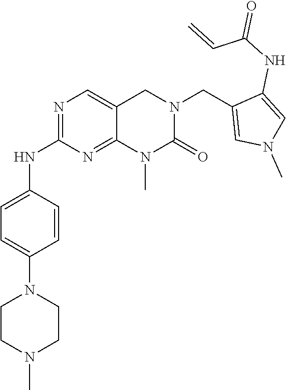 [SMILES: C=CC(=O)Nc1cn(C)cc1CN1Cc2cnc(Nc3ccc(N4CCN(C)CC4)cc3)nc2N(C)C1=O] |
| 039 | 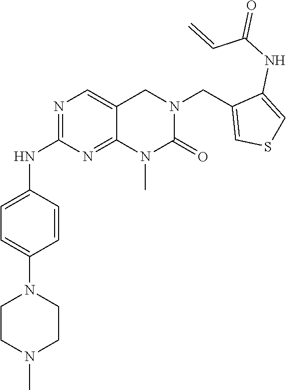 [SMILES: C=CC(=O)Nc1cscc1CN1Cc2cnc(Nc3ccc(N4CCN(C)CC4)cc3)nc2N(C)C1=O] |
| 040 | 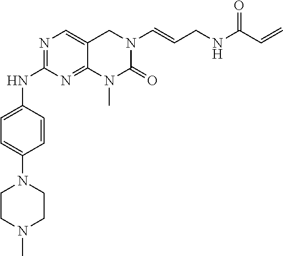 [SMILES: C=CC(=O)NC/C=C/N1Cc2cnc(Nc3ccc(N4CCN(C)CC4)cc3)nc2N(C)C1=O] |
| 041 | 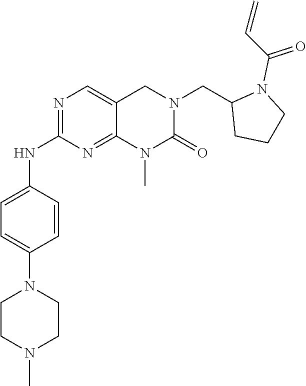 [SMILES: C=CC(=O)N1CCCC1CN1Cc2cnc(Nc3ccc(N4CCN(C)CC4)cc3)nc2N(C)C1=O] |
| 042 | 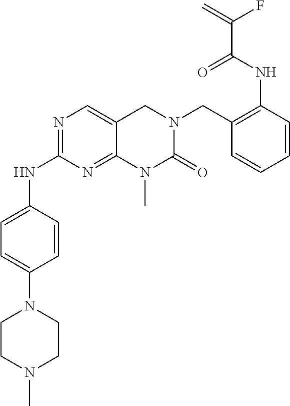 [SMILES: C=C(F)C(=O)Nc1ccccc1CN1Cc2cnc(Nc3ccc(N4CCN(C)CC4)cc3)nc2N(C)C1=O] |
| 043 | 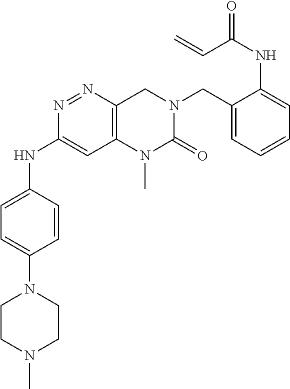 [SMILES: C=CC(=O)Nc1ccccc1CN1Cc2nnc(Nc3ccc(N4CCN(C)CC4)cc3)cc2N(C)C1=O] |
| 044 | 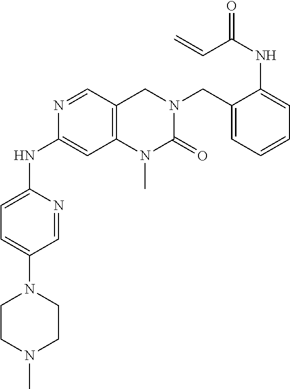 [SMILES: C=CC(=O)Nc1ccccc1CN1Cc2cnc(Nc3ccc(N4CCN(C)CC4)cn3)cc2N(C)C1=O] |
| 045 | 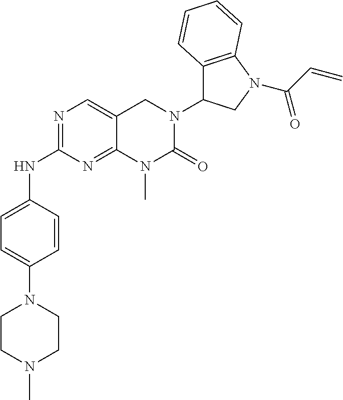 [SMILES: C=CC(=O)N1CC(N2Cc3cnc(Nc4ccc(N5CCN(C)CC5)cc4)nc3N(C)C2=O)c2ccccc21] |

or a pharmaceutically acceptable salt thereof.

In another embodiment, the compound is selected from the group consisting of a compound in Table 1a.

TABLE 1a

| Compound | |
|---|---|
| No. | Structure |
| 046 | 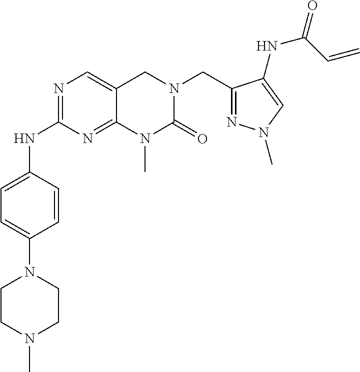 [SMILES: C=CC(=O)Nc1cn(C)nc1CN1Cc2cnc(Nc3ccc(N4CCN(C)CC4)cc3)nc2N(C)C1=O] |
| 047 | 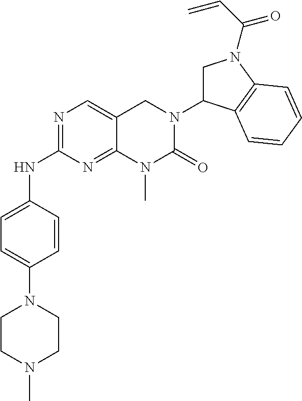 [SMILES: C=CC(=O)N1CC(N2Cc3cnc(Nc4ccc(N5CCN(C)CC5)cc4)nc3N(C)C2=O)c2ccccc21] |
| 048 | 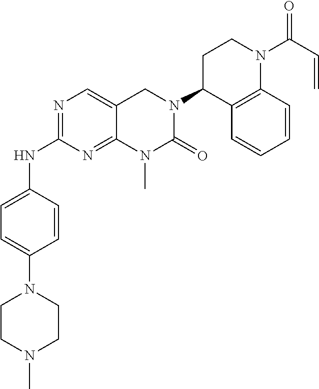 [SMILES: C=CC(=O)N1CC[C@H](N2Cc3cnc(Nc4ccc(N5CCN(C)CC5)cc4)nc3N(C)C2=O)c2ccccc21] |
| 049 | 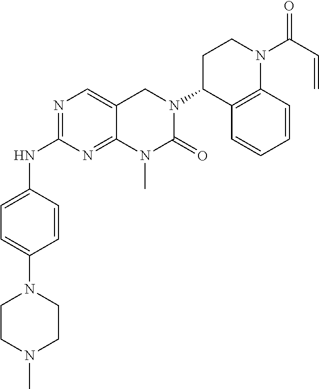 [SMILES: C=CC(=O)N1CC[C@@H](N2Cc3cnc(Nc4ccc(N5CCN(C)CC5)cc4)nc3N(C)C2=O)c2ccccc21] |
| 050 | 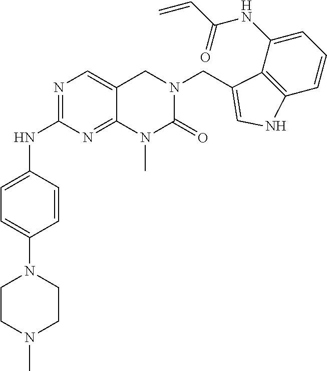 [SMILES: C=CC(=O)Nc1cccc2[nH]cc(CN3Cc4cnc(Nc5ccc(N6CCN(C)CC6)cc5)nc4N(C)C3=O)c12] |
| 051 | 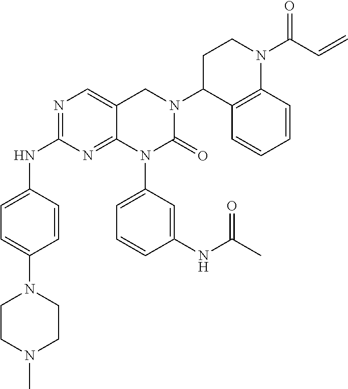 [SMILES: C=CC(=O)N1CCC(N2Cc3cnc(Nc4ccc(N5CCN(C)CC5)cc4)nc3N(c3cccc(NC(C)=O)c3)C2=O)c2ccccc21] |
| 052 | 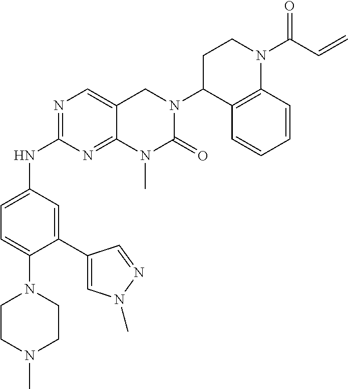 [SMILES: C=CC(=O)N1CCC(N2Cc3cnc(Nc4ccc(N5CCN(C)CC5)c(-c5cnn(C)c5)c4)nc3N(C)C2=O)c2ccccc21] |
| 053 | 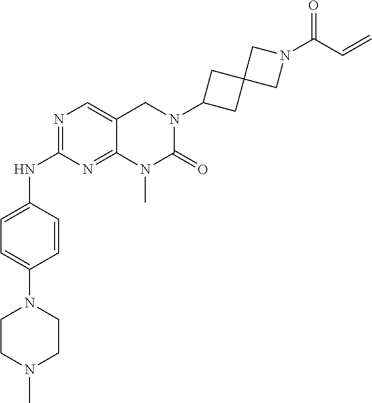 [SMILES: C=CC(=O)N1CC2(CC(N3Cc4cnc(Nc5ccc(N6CCN(C)CC6)cc5)nc4N(C)C3=O)C2)C1] |
| 054 | 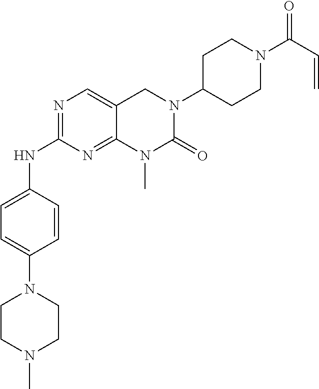 [SMILES: C=CC(=O)N1CCC(N2Cc3cnc(Nc4ccc(N5CCN(C)CC5)cc4)nc3N(C)C2=O)CC1] |
| 055 | 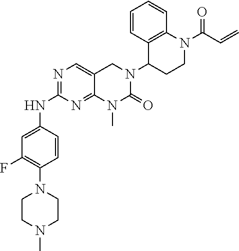 [SMILES: C=CC(=O)N1CCC(N2Cc3cnc(Nc4ccc(N5CCN(C)CC5)c(F)c4)nc3N(C)C2=O)c2ccccc21] |
| 056 | 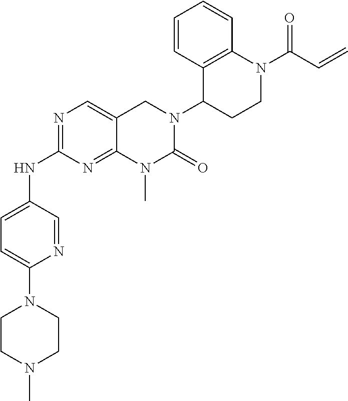 [SMILES: C=CC(=O)N1CCC(N2Cc3cnc(Nc4ccc(N5CCN(C)CC5)nc4)nc3N(C)C2=O)c2ccccc21] |
| 057 | 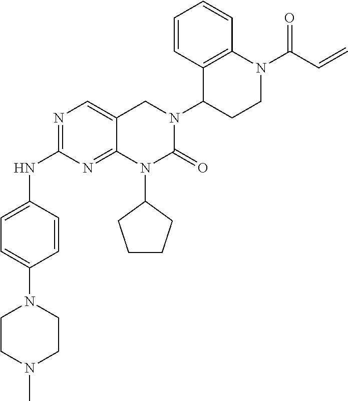 [SMILES: C=CC(=O)N1CCC(N2Cc3cnc(Nc4ccc(N5CCN(C)CC5)cc4)nc3N(C3CCCC3)C2=O)c2ccccc21] |
| 058 | 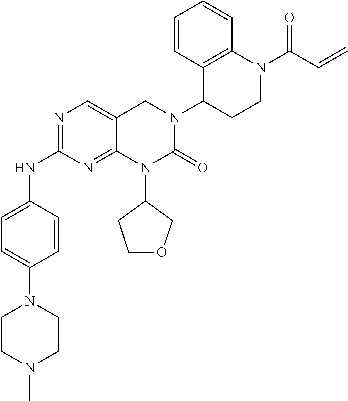 [SMILES: C=CC(=O)N1CCC(N2Cc3cnc(Nc4ccc(N5CCN(C)CC5)cc4)nc3N(C3CCOC3)C2=O)c2ccccc21] |
| 059 | 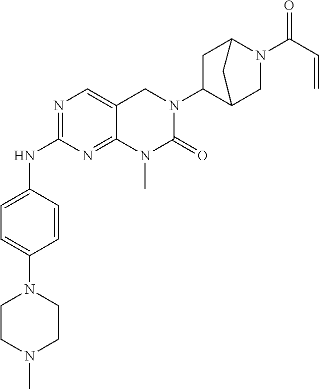 [SMILES: C=CC(=O)N1CC2CC1CC2N1Cc2cnc(Nc3ccc(N4CCN(C)CC4)cc3)nc2N(C)C1=O] |
| 060 | 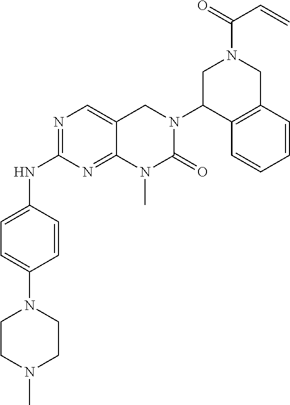 [SMILES: C=CC(=O)N1Cc2ccccc2C(N2Cc3cnc(Nc4ccc(N5CCN(C)CC5)cc4)nc3N(C)C2=O)C1] |
| 061 | 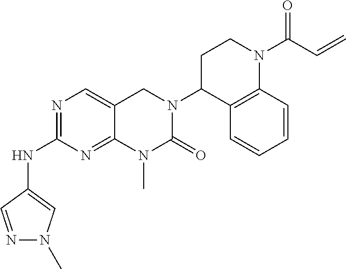 [SMILES: C=CC(=O)N1CCC(N2Cc3cnc(Nc4cnn(C)c4)nc3N(C)C2=O)c2ccccc21] |
| 062 | 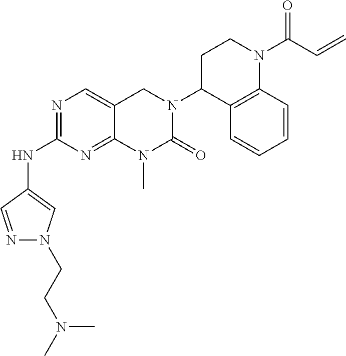 [SMILES: C=CC(=O)N1CCC(N2Cc3cnc(Nc4cnn(CCN(C)C)c4)nc3N(C)C2=O)c2ccccc21] |
| 063 | 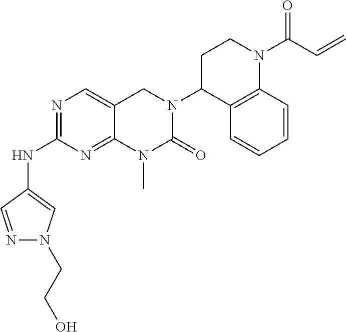 [SMILES: C=CC(=O)N1CCC(N2Cc3cnc(Nc4cnn(CCO)c4)nc3N(C)C2=O)c2ccccc21] |
| 064 | 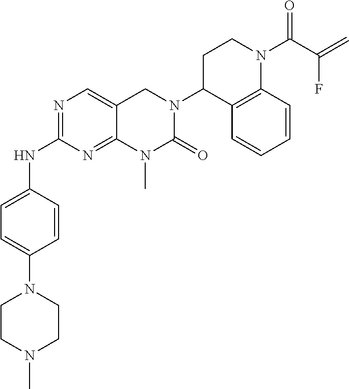 [SMILES: C=C(F)C(=O)N1CCC(N2Cc3cnc(Nc4ccc(N5CCN(C)CC5)cc4)nc3N(C)C2=O)c2ccccc21] |
| 065 | 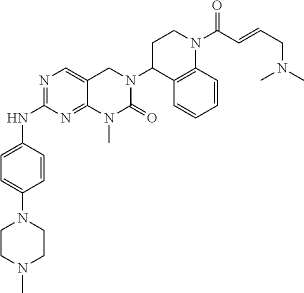 [SMILES: CN(C)C/C=C/C(=O)N1CCC(N2Cc3cnc(Nc4ccc(N5CCN(C)CC5)cc4)nc3N(C)C2=O)c2ccccc21] |
| 066 | 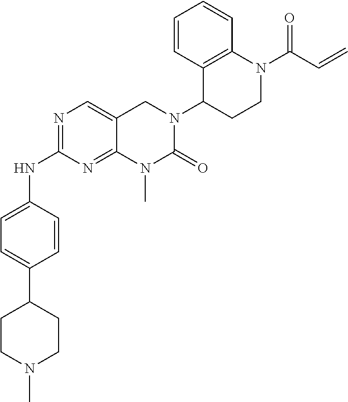 [SMILES: C=CC(=O)N1CCC(N2Cc3cnc(Nc4ccc(C5CCN(C)CC5)cc4)nc3N(C)C2=O)c2ccccc21] |
| 067 | 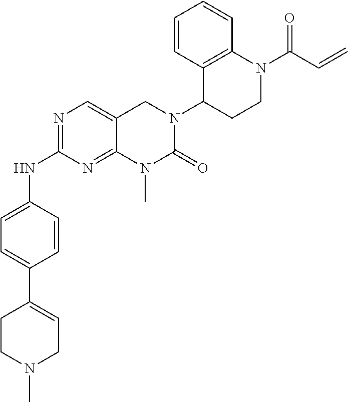 [SMILES: C=CC(=O)N1CCC(N2Cc3cnc(Nc4ccc(C5=CCN(C)CC5)cc4)nc3N(C)C2=O)c2ccccc21] |
| 068 | 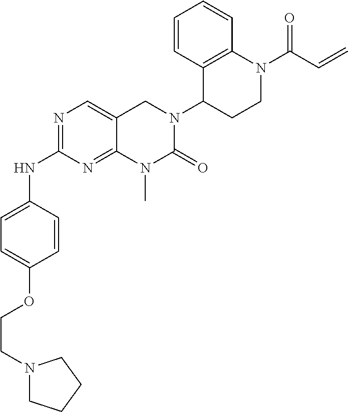 [SMILES: C=CC(=O)N1CCC(N2Cc3cnc(Nc4ccc(OCCN5CCCC5)cc4)nc3N(C)C2=O)c2ccccc21] |
| 069 | 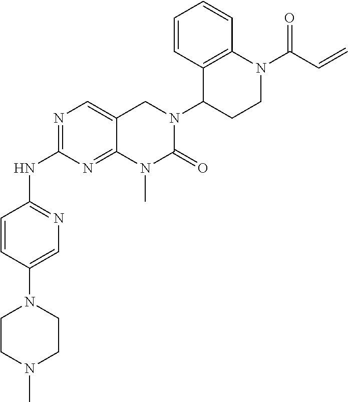 [SMILES: C=CC(=O)N1CCC(N2Cc3cnc(Nc4ccc(N5CCN(C)CC5)cn4)nc3N(C)C2=O)c2ccccc21] |
| 070 | 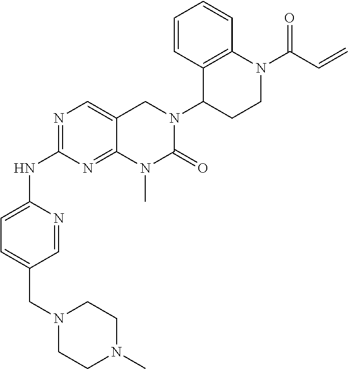 [SMILES: C=CC(=O)N1CCC(N2Cc3cnc(Nc4ccc(CN5CCN(C)CC5)cn4)nc3N(C)C2=O)c2ccccc21] |
| 071 | 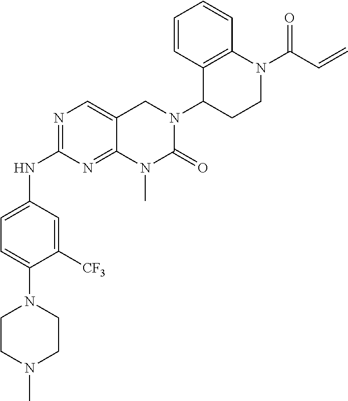 [SMILES: C=CC(=O)N1CCC(N2Cc3cnc(Nc4ccc(N5CCN(C)CC5)c(C(F)(F)F)c4)nc3N(C)C2=O)c2ccccc21] |
| 072 | 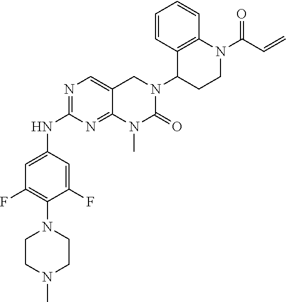 [SMILES: C=CC(=O)N1CCC(N2Cc3cnc(Nc4cc(F)c(N5CCN(C)CC5)c(F)c4)nc3N(C)C2=O)c2ccccc21] |
| 073 | 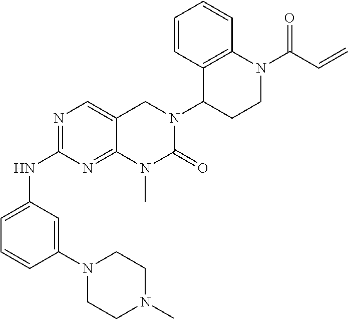 [SMILES: C=CC(=O)N1CCC(N2Cc3cnc(Nc4cccc(N5CCN(C)CC5)c4)nc3N(C)C2=O)c2ccccc21] |
| 074 | 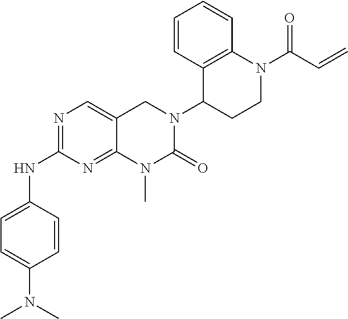 [SMILES: C=CC(=O)N1CCC(N2Cc3cnc(Nc4ccc(N(C)C)cc4)nc3N(C)C2=O)c2ccccc21] |
| 075 | 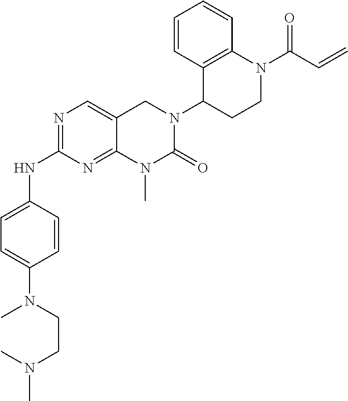 [SMILES: C=CC(=O)N1CCC(N2Cc3cnc(Nc4ccc(N(C)CCN(C)C)cc4)nc3N(C)C2=O)c2ccccc21] |
| 076 | 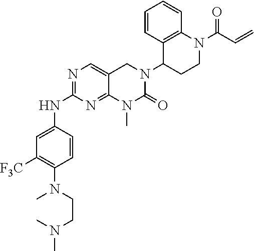 [SMILES: C=CC(=O)N1CCC(N2Cc3cnc(Nc4ccc(N(C)CCN(C)C)c(C(F)(F)F)c4)nc3N(C)C2=O)c2ccccc21] |
| 077 | 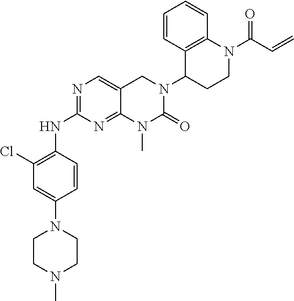 [SMILES: C=CC(=O)N1CCC(N2Cc3cnc(Nc4ccc(N5CCN(C)CC5)cc4Cl)nc3N(C)C2=O)c2ccccc21] |
| 078 | 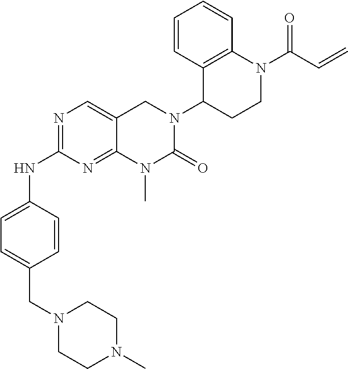 [SMILES: C=CC(=O)N1CCC(N2Cc3cnc(Nc4ccc(CN5CCN(C)CC5)cc4)nc3N(C)C2=O)c2ccccc21] |
| 079 | 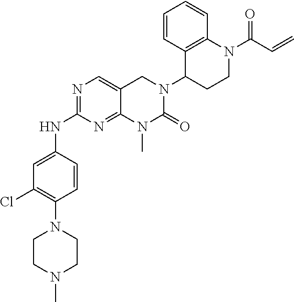 [SMILES: C=CC(=O)N1CCC(N2Cc3cnc(Nc4ccc(N5CCN(C)CC5)c(Cl)c4)nc3N(C)C2=O)c2ccccc21] |
| 080 | 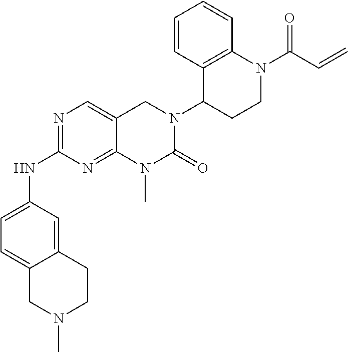 [SMILES: C=CC(=O)N1CCC(N2Cc3cnc(Nc4ccc5c(c4)CCN(C)C5)nc3N(C)C2=O)c2ccccc21] |
| 081 | 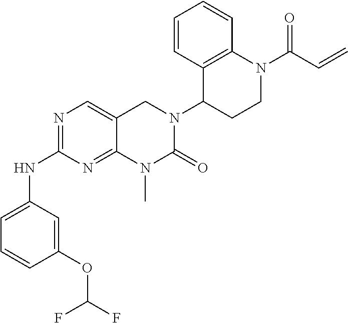 [SMILES: C=CC(=O)N1CCC(N2Cc3cnc(Nc4cccc(OC(F)F)c4)nc3N(C)C2=O)c2ccccc21] |
| 082 | 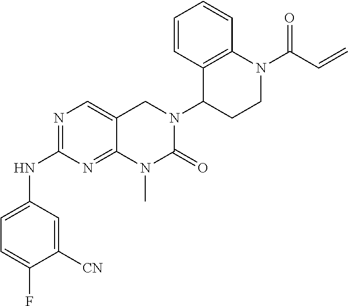 [SMILES: C=CC(=O)N1CCC(N2Cc3cnc(Nc4ccc(F)c(C#N)c4)nc3N(C)C2=O)c2ccccc21] |
| 083 | 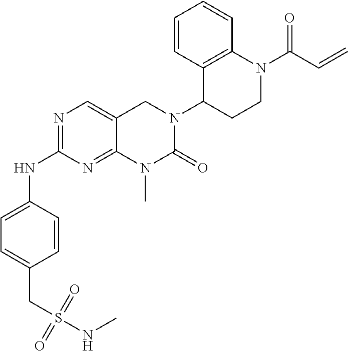 [SMILES: C=CC(=O)N1CCC(N2Cc3cnc(Nc4ccc(CS(=O)(=O)NC)cc4)nc3N(C)C2=O)c2ccccc21] |
| 084 | 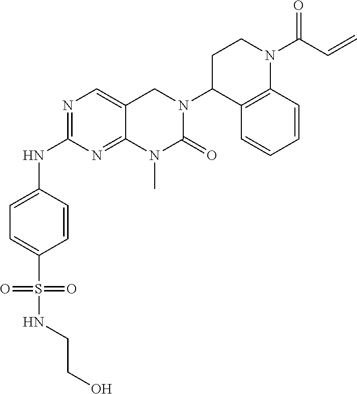 [SMILES: C=CC(=O)N1CCC(N2Cc3cnc(Nc4ccc(S(=O)(=O)NCCO)cc4)nc3N(C)C2=O)c2ccccc21] |
| 085 | 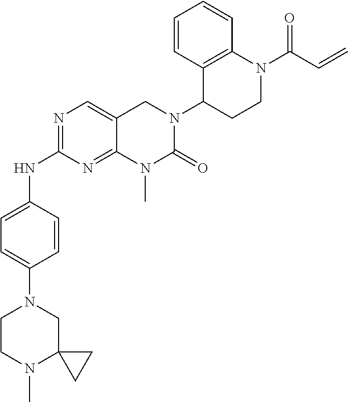 [SMILES: C=CC(=O)N1CCC(N2Cc3cnc(Nc4ccc(N5CCN(C)C6(CC6)C5)cc4)nc3N(C)C2=O)c2ccccc21] |
| 086 | 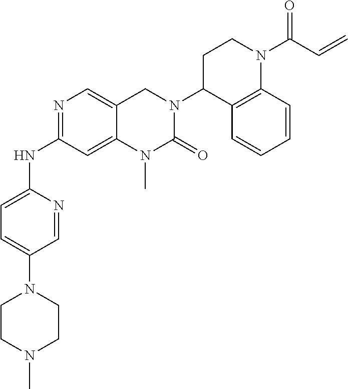 [SMILES: C=CC(=O)N1CCC(N2Cc3cnc(Nc4ccc(N5CCN(C)CC5)cn4)cc3N(C)C2=O)c2ccccc21] |
| 087 | 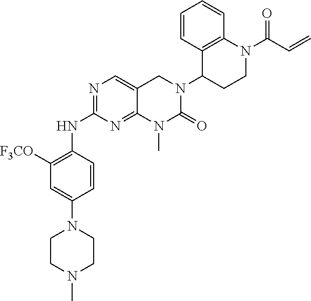 [SMILES: C=CC(=O)N1CCC(N2Cc3cnc(Nc4ccc(N5CCN(C)CC5)cc4OC(F)(F)F)nc3N(C)C2=O)c2ccccc21] |
| 088 | 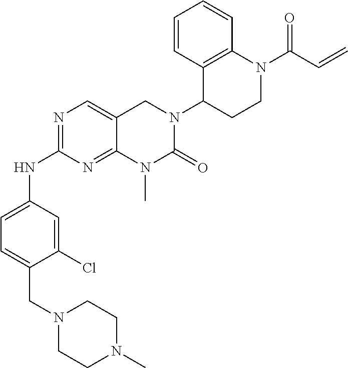 [SMILES: C=CC(=O)N1CCC(N2Cc3cnc(Nc4ccc(CN5CCN(C)CC5)c(Cl)c4)nc3N(C)C2=O)c2ccccc21] |
| 089 | 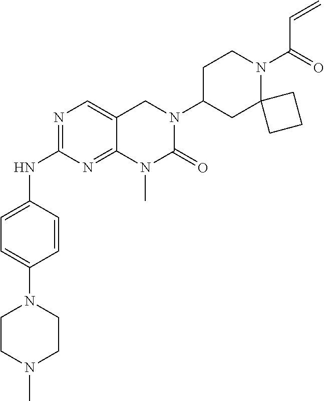 [SMILES: C=CC(=O)N1CCC(N2Cc3cnc(Nc4ccc(N5CCN(C)CC5)cc4)nc3N(C)C2=O)CC12CCC2] |
| 090 | 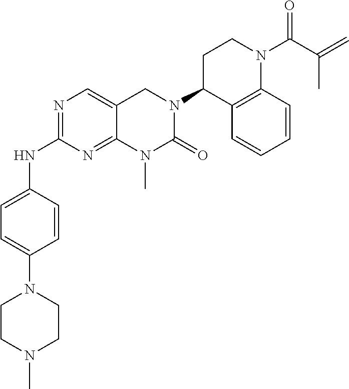 [SMILES: C=C(C)C(=O)N1CC[C@H](N2Cc3cnc(Nc4ccc(N5CCN(C)CC5)cc4)nc3N(C)C2=O)c2ccccc21] |
| 091 | 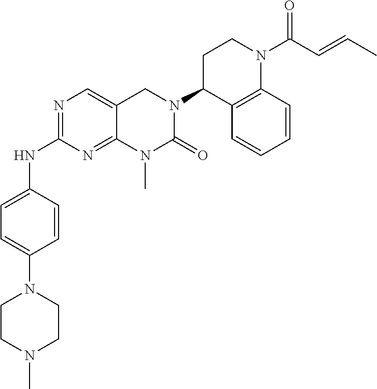 [SMILES: C/C=C/C(=O)N1CC[C@H](N2Cc3cnc(Nc4ccc(N5CCN(C)CC5)cc4)nc3N(C)C2=O)c2ccccc21] |
| 092 | 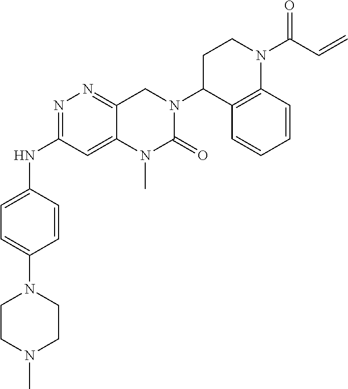 [SMILES: C=CC(=O)N1CCC(N2Cc3nnc(Nc4ccc(N5CCN(C)CC5)cc4)cc3N(C)C2=O)c2ccccc21] |
| 093 | 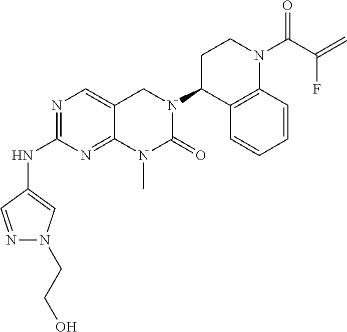 [SMILES: C=C(F)C(=O)N1CC[C@H](N2Cc3cnc(Nc4cnn(CCO)c4)nc3N(C)C2=O)c2ccccc21] |
| 094 | 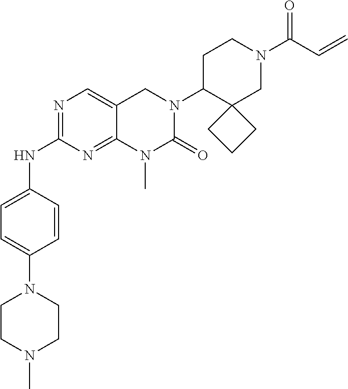 [SMILES: C=CC(=O)N1CCC(N2Cc3cnc(Nc4ccc(N5CCN(C)CC5)cc4)nc3N(C)C2=O)C2(CCC2)C1] |
| 095 | 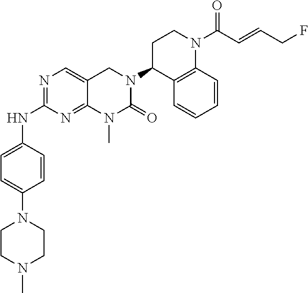 [SMILES: CN1CCN(c2ccc(Nc3ncc4c(n3)N(C)C(=O)N([C@H]3CCN(C(=O)/C=C/CF)c5ccccc53)C4)cc2)CC1] |
| 096 | 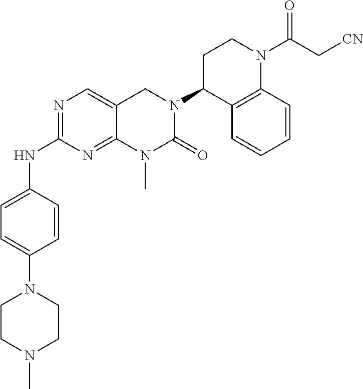 [SMILES: CN1CCN(c2ccc(Nc3ncc4c(n3)N(C)C(=O)N([C@H]3CCN(C(=O)CC#N)c5ccccc53)C4)cc2)CC1] |
| 097 | 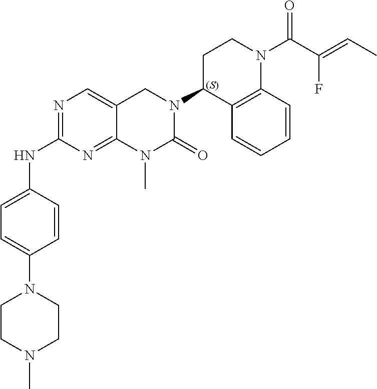 |
| 098 | 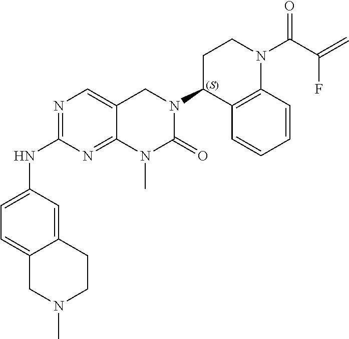 |
| 099 | 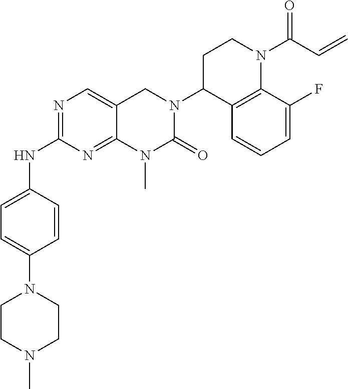 [SMILES: C=CC(=O)N1CCC(N2Cc3cnc(Nc4ccc(N5CCN(C)CC5)cc4)nc3N(C)C2=O)c2cccc(F)c21] |
| 100 | 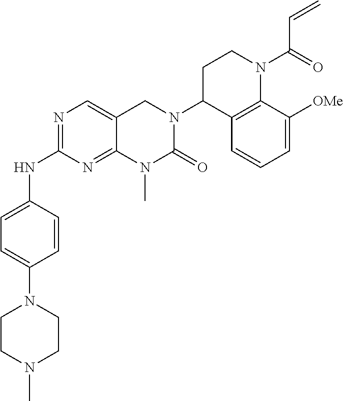 [SMILES: C=CC(=O)N1CCC(N2Cc3cnc(Nc4ccc(N5CCN(C)CC5)cc4)nc3N(C)C2=O)c2cccc(OC)c21] |
| 101 | 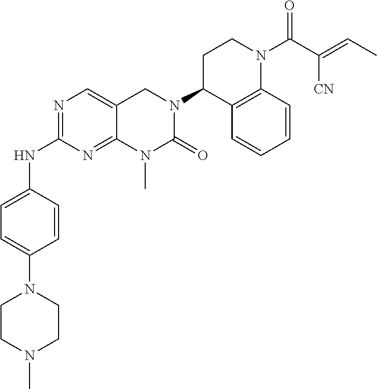 [SMILES: C/C=C(\C#N)C(=O)N1CC[C@H](N2Cc3cnc(Nc4ccc(N5CCN(C)CC5)cc4)nc3N(C)C2=O)c2ccccc21] |
| 102 | 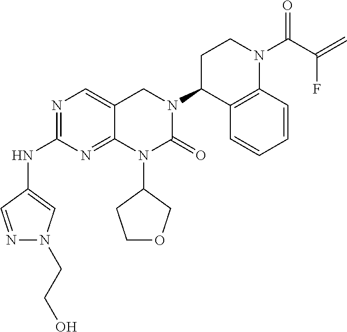 [SMILES: C=C(F)C(=O)N1CC[C@H](N2Cc3cnc(Nc4cnn(CCO)c4)nc3N(C3CCOC3)C2=O)c2ccccc21] |
| 103 | 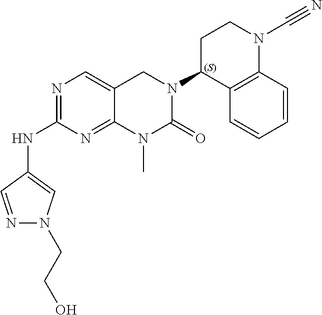 |
| 104 | 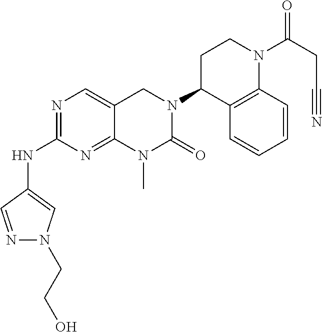 [SMILES: CN1C(=O)N([C@H]2CCN(C(=O)CC#N)c3ccccc32)Cc2cnc(Nc3cnn(CCO)c3)nc21] |
| 105 | 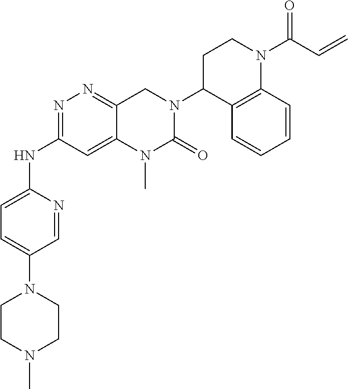 [SMILES: C=CC(=O)N1CCC(N2Cc3nnc(Nc4ccc(N5CCN(C)CC5)cn4)cc3N(C)C2=O)c2ccccc21] |
| 106 | 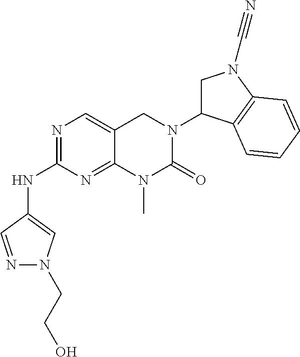 [SMILES: CN1C(=O)N(C2CN(C#N)c3ccccc32)Cc2cnc(Nc3cnn(CCO)c3)nc21] |
| 107 | 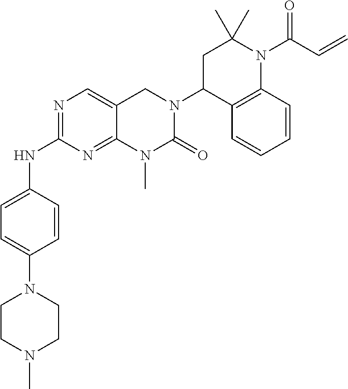 [SMILES: C=CC(=O)N1c2ccccc2C(N2Cc3cnc(Nc4ccc(N5CCN(C)CC5)cc4)nc3N(C)C2=O)CC1(C)C] |
| 108 | 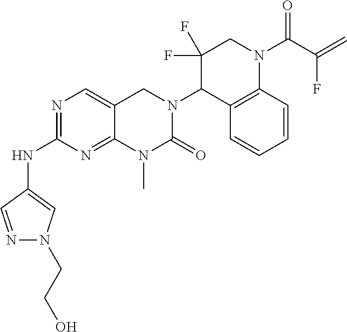 [SMILES: C=C(F)C(=O)N1CC(F)(F)C(N2Cc3cnc(Nc4cnn(CCO)c4)nc3N(C)C2=O)c2ccccc21] |
| 109 | 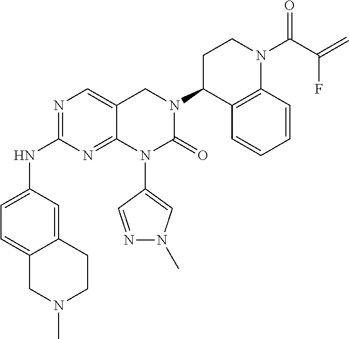 [SMILES: C=C(F)C(=O)N1CC[C@H](N2Cc3cnc(Nc4ccc5c(c4)CCN(C)C5)nc3N(c3cnn(C)c3)C2=O)c2ccccc21] |
| 110 | 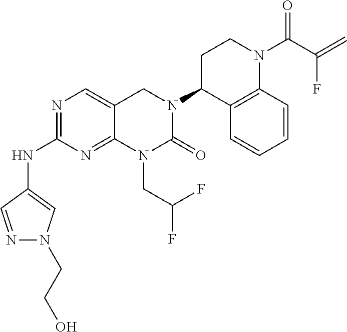 [SMILES: C=C(F)C(=O)N1CC[C@H](N2Cc3cnc(Nc4cnn(CCO)c4)nc3N(CC(F)F)C2=O)c2ccccc21] |
| 111 | 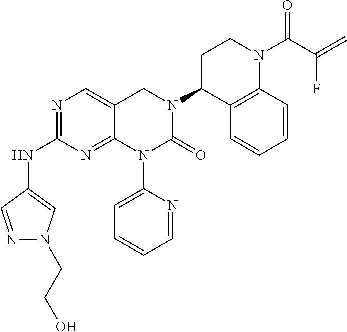 [SMILES: C=C(F)C(=O)N1CC[C@H](N2Cc3cnc(Nc4cnn(CCO)c4)nc3N(c3ccccn3)C2=O)c2ccccc21] |
| 112 | 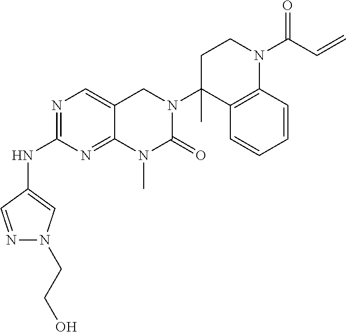 [SMILES: C=CC(=O)N1CCC(C)(N2Cc3cnc(Nc4cnn(CCO)c4)nc3N(C)C2=O)c2ccccc21] |
| 113 | 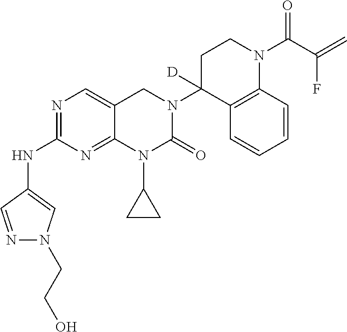 [SMILES: [2H]C1(N2Cc3cnc(Nc4cnn(CCO)c4)nc3N(C3CC3)C2=O)CCN(C(=O)C(=C)F)c2ccccc21] |
| 114 | 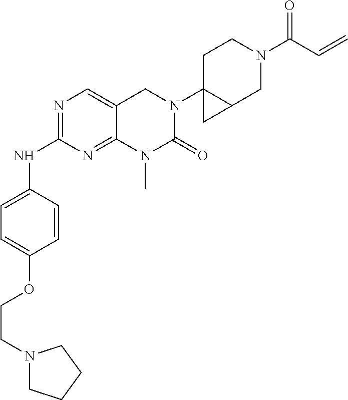 [SMILES: C=CC(=O)N1CCC2(N3Cc4cnc(Nc5ccc(OCCN6CCCC6)cc5)nc4N(C)C3=O)CC2C1] |
| 115 | 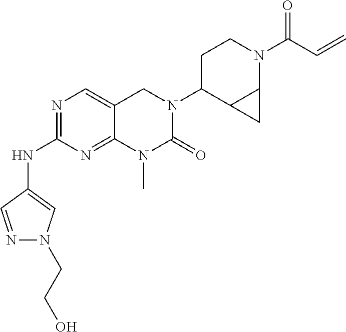 [SMILES: C=CC(=O)N1CCC(N2Cc3cnc(Nc4cnn(CCO)c4)nc3N(C)C2=O)C2CC21] |
| 116 | 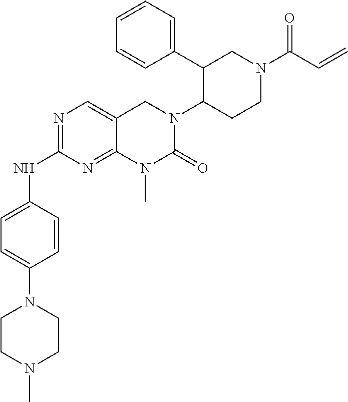 [SMILES: C=CC(=O)N1CCC(N2Cc3cnc(Nc4ccc(N5CCN(C)CC5)cc4)nc3N(C)C2=O)C(c2ccccc2)C1] |

or a pharmaceutically acceptable salt thereof.

In yet another embodiment, the compound is selected from the group consisting of a compound in Table 1b.

TABLE 1b

| Compound | |
|---|---|
| No. | Structure |
| 117 | 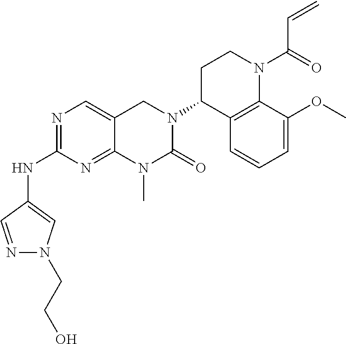 [SMILES: C=CC(=O)N1CC[C@@H](N2Cc3cnc(Nc4cnn(CCO)c4)nc3N(C)C2=O)c2cccc(OC)c21] |
| 118 | 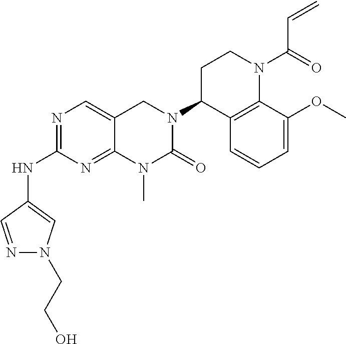 [SMILES: C=CC(=O)N1CC[C@H](N2Cc3cnc(Nc4cnn(CCO)c4)nc3N(C)C2=O)c2cccc(OC)c21] |
| 119 | 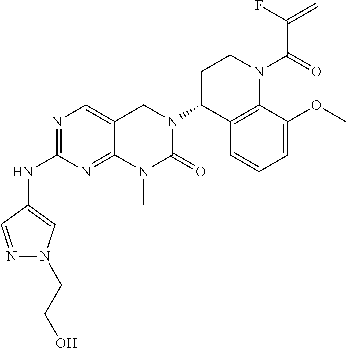 [SMILES: C=C(F)C(=O)N1CC[C@@H](N2Cc3cnc(Nc4cnn(CCO)c4)nc3N(C)C2=O)c2cccc(OC)c21] |
| 120 | 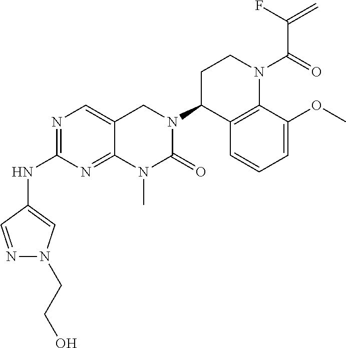 [SMILES: C=C(F)C(=O)N1CC[C@H](N2Cc3cnc(Nc4cnn(CCO)c4)nc3N(C)C2=O)c2cccc(OC)c21] |
| 121 | 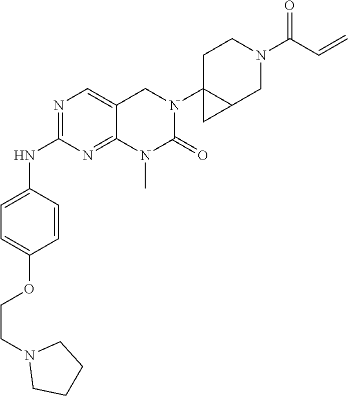 [SMILES: C=CC(=O)N1CCC2(N3Cc4cnc(Nc5ccc(OCCN6CCCC6)cc5)nc4N(C)C3=O)CC2C1] |
| 122 | 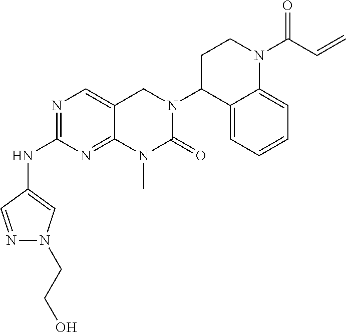 [SMILES: C=CC(=O)N1CCC(N2Cc3cnc(Nc4cnn(CCO)c4)nc3N(C)C2=O)c2ccccc21] |
| 123 | 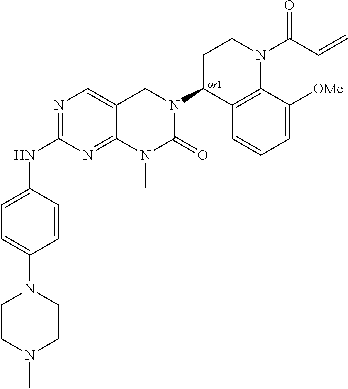 [SMILES: C=CC(=O)N1CC[C@H](N2Cc3cnc(Nc4ccc(N5CCN(C)CC5)cc4)nc3N(C)C2=O)c2cccc(OC)c21] |
| 124 | 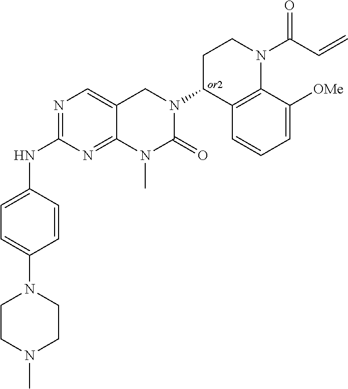 [SMILES: C=CC(=O)N1CC[C@@H](N2Cc3cnc(Nc4ccc(N5CCN(C)CC5)cc4)nc3N(C)C2=O)c2cccc(OC)c21] |
| 125 | 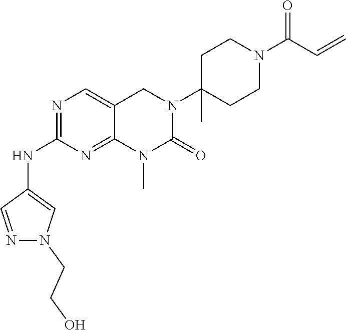 [SMILES: C=CC(=O)N1CCC(C)(N2Cc3cnc(Nc4cnn(CCO)c4)nc3N(C)C2=O)CC1] |
| 126 | 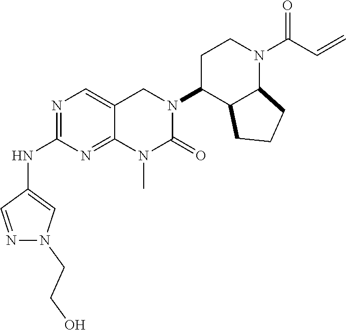 |
| 127 | 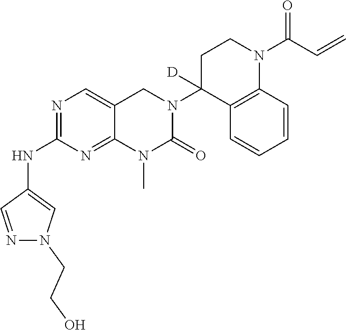 [SMILES: [2H]C1(N2Cc3cnc(Nc4cnn(CCO)c4)nc3N(C)C2=O)CCN(C(=O)C=C)c2ccccc21] |
| 128 | 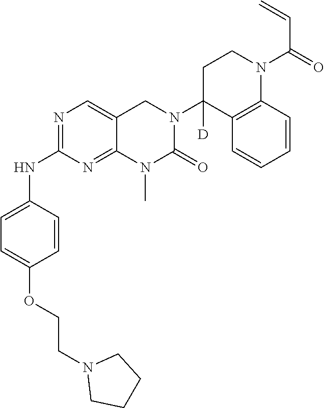 [SMILES: [2H]C1(N2Cc3cnc(Nc4ccc(OCCN5CCCC5)cc4)nc3N(C)C2=O)CCN(C(=O)C=C)c2ccccc21] |
| 129 | 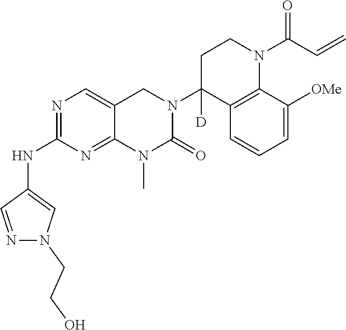 [SMILES: [2H]C1(N2Cc3cnc(Nc4cnn(CCO)c4)nc3N(C)C2=O)CCN(C(=O)C=C)c2c(OC)cccc21] |
| 130 | 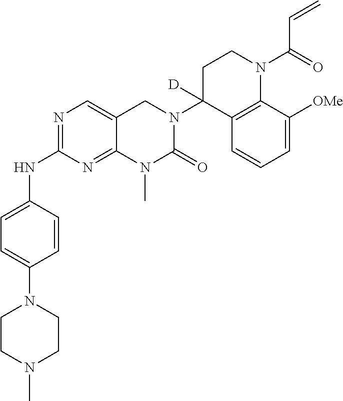 [SMILES: [2H]C1(N2Cc3cnc(Nc4ccc(N5CCN(C)CC5)cc4)nc3N(C)C2=O)CCN(C(=O)C=C)c2c(OC)cccc21] |
| 131 | 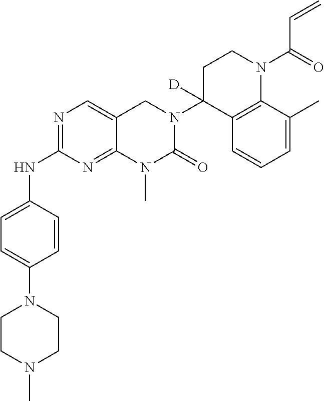 [SMILES: [2H]C1(N2Cc3cnc(Nc4ccc(N5CCN(C)CC5)cc4)nc3N(C)C2=O)CCN(C(=O)C=C)c2c(C)cccc21] |
| 132 | 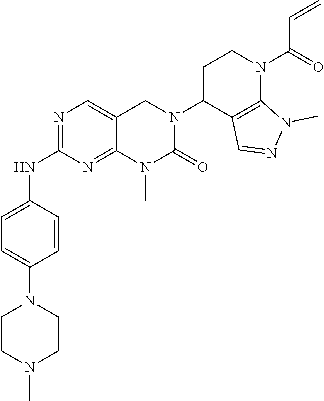 [SMILES: C=CC(=O)N1CCC(N2Cc3cnc(Nc4ccc(N5CCN(C)CC5)cc4)nc3N(C)C2=O)c2cnn(C)c21] |
| 133 | 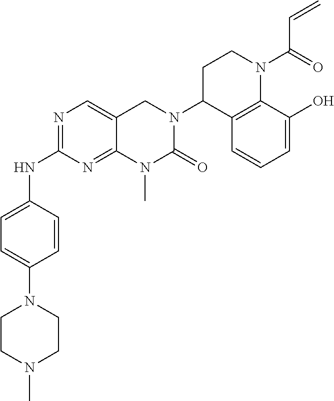 [SMILES: C=CC(=O)N1CCC(N2Cc3cnc(Nc4ccc(N5CCN(C)CC5)cc4)nc3N(C)C2=O)c2cccc(O)c21] |
| 134 | 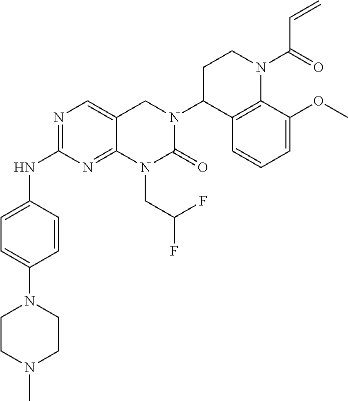 [SMILES: C=CC(=O)N1CCC(N2Cc3cnc(Nc4ccc(N5CCN(C)CC5)cc4)nc3N(CC(F)F)C2=O)c2cccc(OC)c21] |
| 135 | 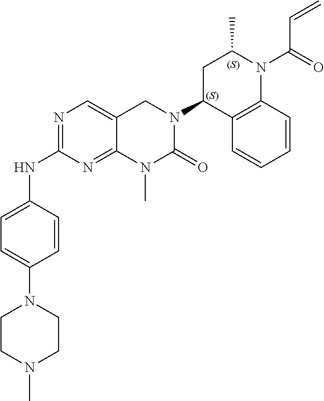 |
| 136 | 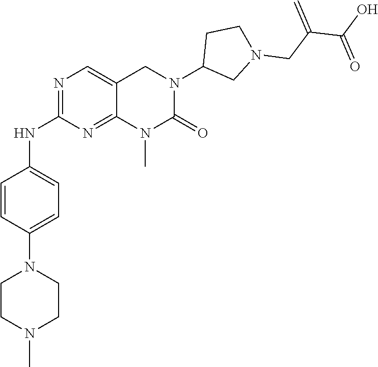 [SMILES: C=C(CN1CCC(N2Cc3cnc(Nc4ccc(N5CCN(C)CC5)cc4)nc3N(C)C2=O)C1)C(=O)O] |
| 137 | 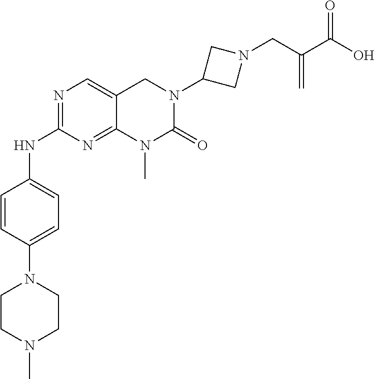 [SMILES: C=C(CN1CC(N2Cc3cnc(Nc4ccc(N5CCN(C)CC5)cc4)nc3N(C)C2=O)C1)C(=O)O] |
| 138 | 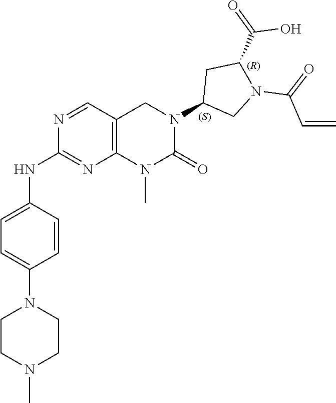 |
| 139 | 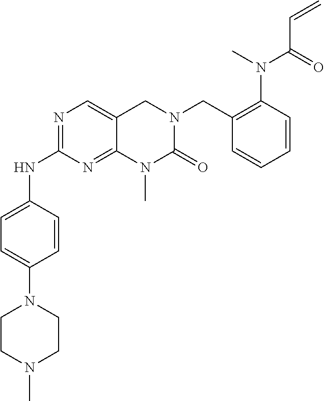 [SMILES: C=CC(=O)N(C)c1ccccc1CN1Cc2cnc(Nc3ccc(N4CCN(C)CC4)cc3)nc2N(C)C1=O] |
| 140 | 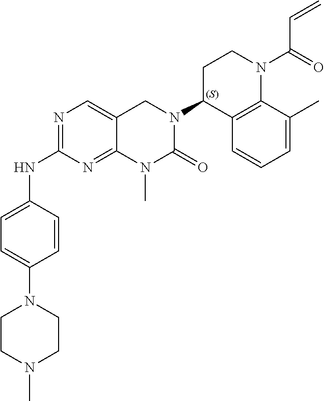 |
| 141 | 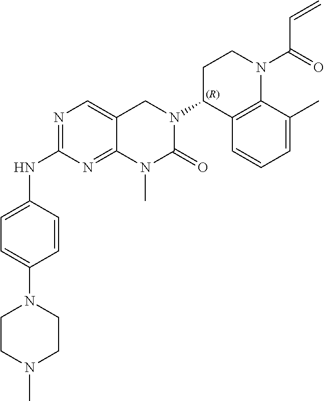 |
| 142 | 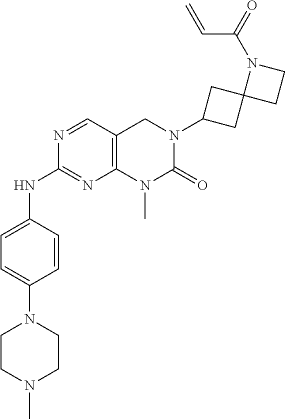 [SMILES: C=CC(=O)N1CCC12CC(N1Cc3cnc(Nc4ccc(N5CCN(C)CC5)cc4)nc3N(C)C1=O)C2] |
| 143 | 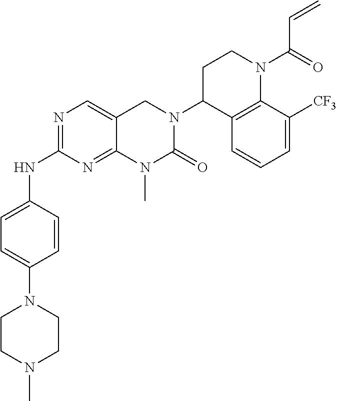 [SMILES: C=CC(=O)N1CCC(N2Cc3cnc(Nc4ccc(N5CCN(C)CC5)cc4)nc3N(C)C2=O)c2cccc(C(F)(F)F)c21] |
| 144 | 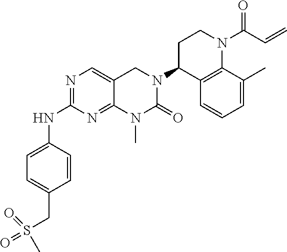 [SMILES: C=CC(=O)N1CC[C@H](N2Cc3cnc(Nc4ccc(CS(C)(=O)=O)cc4)nc3N(C)C2=O)c2cccc(C)c21] |
| 145 | 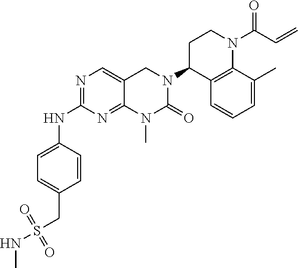 [SMILES: C=CC(=O)N1CC[C@H](N2Cc3cnc(Nc4ccc(CS(=O)(=O)NC)cc4)nc3N(C)C2=O)c2cccc(C)c21] |
| 146 | 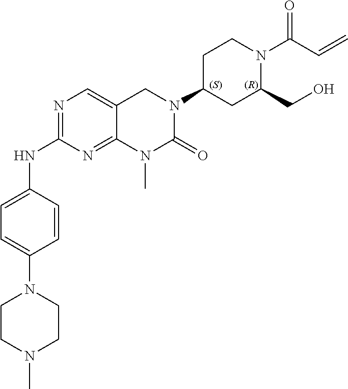 |
| 147 | 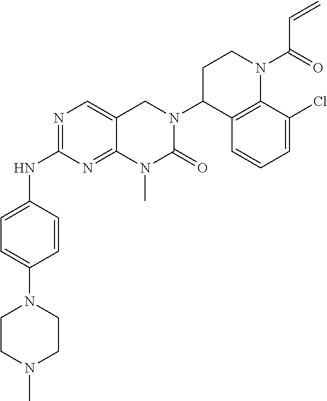 [SMILES: C=CC(=O)N1CCC(N2Cc3cnc(Nc4ccc(N5CCN(C)CC5)cc4)nc3N(C)C2=O)c2cccc(Cl)c21] |
| 148 | 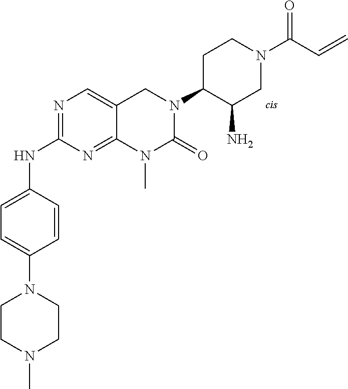 [SMILES: C=CC(=O)N1CC[C@H](N2Cc3cnc(Nc4ccc(N5CCN(C)CC5)cc4)nc3N(C)C2=O)[C@H](N)C1] |
| 149 | 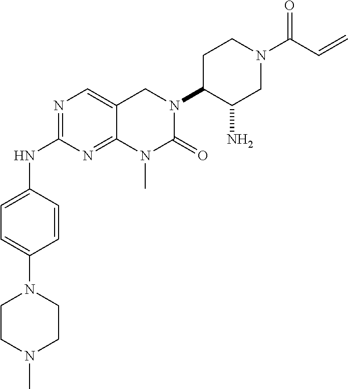 |
| 150 | 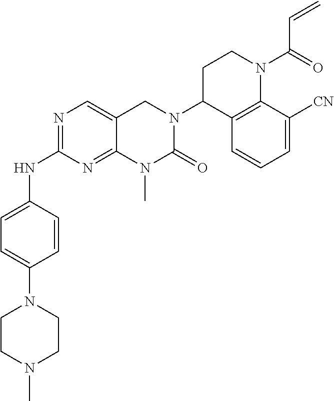 [SMILES: C=CC(=O)N1CCC(N2Cc3cnc(Nc4ccc(N5CCN(C)CC5)cc4)nc3N(C)C2=O)c2cccc(C#N)c21] |
| 151 | 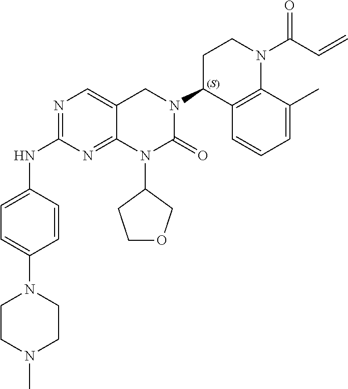 |
| 152 | 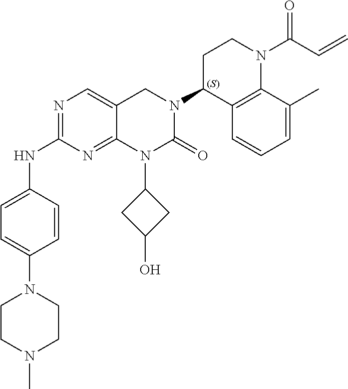 |
| 153 | 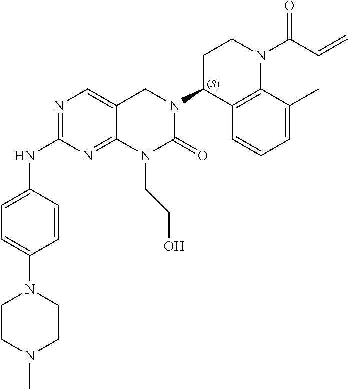 |
| 154 | 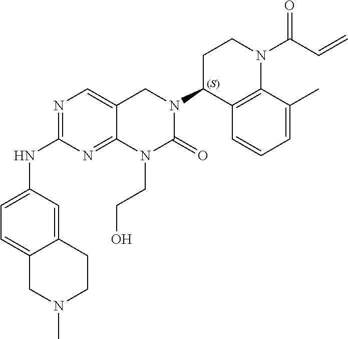 |
| 155 | 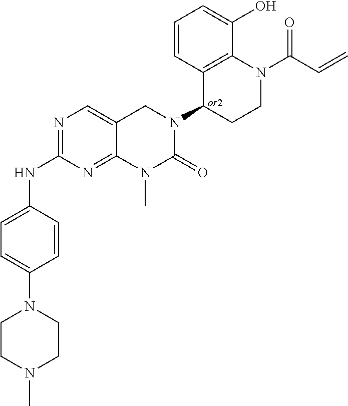 [SMILES: C=CC(=O)N1CC[C@@H](N2Cc3cnc(Nc4ccc(N5CCN(C)CC5)cc4)nc3N(C)C2=O)c2cccc(O)c21] |
| 156 | 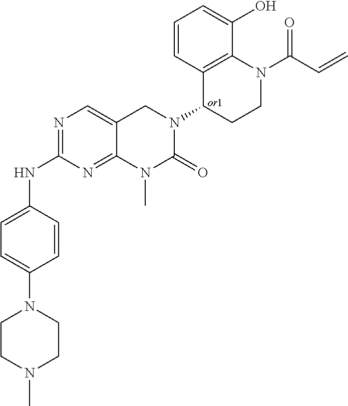 [SMILES: C=CC(=O)N1CC[C@H](N2Cc3cnc(Nc4ccc(N5CCN(C)CC5)cc4)nc3N(C)C2=O)c2cccc(O)c21] |
| 157 | 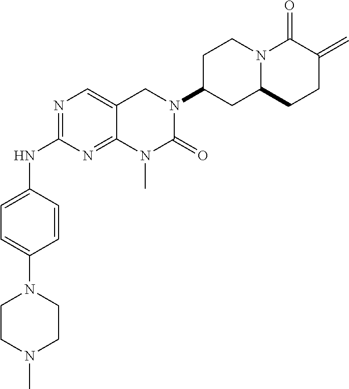 |
| 158 | 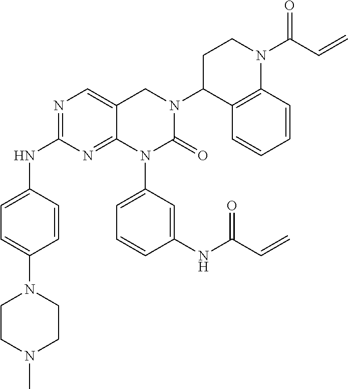 [SMILES: C=CC(=O)Nc1cccc(N2C(=O)N(C3CCN(C(=O)C=C)c4ccccc43)Cc3cnc(Nc4ccc(N5CCN(C)CC5)cc4)nc32)c1] |
| 159 | 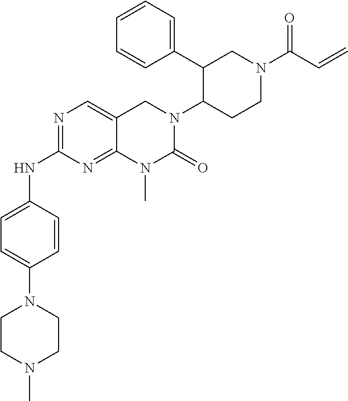 [SMILES: C=CC(=O)N1CCC(N2Cc3cnc(Nc4ccc(N5CCN(C)CC5)cc4)nc3N(C)C2=O)C(c2ccccc2)C1] |

or a pharmaceutically acceptable salt thereof.

In an aspect, the compound is:

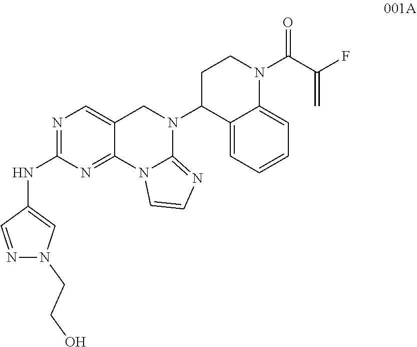 [SMILES: C=C(F)C(=O)N1CCC(N2Cc3cnc(Nc4cnn(CCO)c4)nc3-n3ccnc32)c2ccccc21]

or a pharmaceutically acceptable salt thereof.

The compounds disclosed herein may exist as tautomers and optical isomers (e.g., enantiomers, diastereomers, diastereomeric mixtures, racemic mixtures, and the like).

It is generally well known in the art that any compound that will be converted in vivo to provide a compound disclosed herein is a prodrug within the scope of the present disclosure.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In an aspect, provided herein is a pharmaceutical composition comprising any one of the compounds disclosed herein, or a pharmaceutically acceptable salt thereof, and at least one pharmaceutically acceptable carrier.

In an embodiment, the composition further comprises a second active agent. In another embodiment, the second active agent is selected from the group consisting of a MEK inhibitor, a PI3K inhibitor, and an mTor inhibitor. In yet another embodiment, the second active agent prevents EGFR dimer formation in a subject. In still another embodiment, the second active agent is selected from the group consisting of cetuximab, trastuzumab, and panitumumab. In an embodiment, the second active agent is an ATP competitive EGFR inhibitor. In another embodiment, the ATP competitive EGFR inhibitor is osimertinib, gefitinib, or erlotinib. In another embodiment, the ATP competitive EGFR inhibitor is osimertinib.

In another aspect, provided herein are pharmaceutical compositions comprising a compound of the present disclosure, or a pharmaceutically acceptable salt thereof, and a pharmaceutically acceptable carrier.

In another aspect, provided herein is a method of inhibiting the activity of EGFR, comprising administering to a subject in need thereof an effective amount of a compound of disclosed herein or a pharmaceutical composition comprising a compound disclosed herein and a pharmaceutically acceptable carrier. In an embodiment, the compound targets Cys775 on EGFR.

In another aspect, the pharmaceutical composition further comprises a second active agent, wherein said second active agent prevents EGFR dimer formation, and a pharmaceutically acceptable carrier. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

A compound that binds to an allosteric site in EGFR, such as the compounds of the present disclosure (e.g., the compounds of the formulae disclosed herein), optionally in combination with a second active agent, wherein said second active agent prevents EGFR dimer formation, are capable of modulating EGFR activity. In some embodiments, the compounds of the present disclosure are capable of inhibiting or decreasing EGFR activity without a second active agent (e.g., an antibody such as cetuximab, trastuzumab, or panitumumab). In other embodiments, the compounds of the present disclosure in combination with a second active agent. In an embodiment, the second active agent prevents EGFR dimer formation and/or are capable of inhibiting or decreasing EGFR activity. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

### Methods of Treatment

In an aspect, provided herein is a method of treating cancer in an individual in need thereof, comprising administering to the individual a therapeutically effective amount of a compound disclosed herein, or a pharmaceutically acceptable salt thereof. In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another aspect, provided herein is a method of inhibiting the activity of EGFR in an subject in need thereof, comprising administering to the subject a therapeutically effective amount of a compound provided herein, or a pharmaceutically acceptable salt thereof.

In yet another aspect, provided herein is a method of inhibiting a kinase in an individual in need thereof, comprising administering to the individual a therapeutically effective amount of a compound provided herein. In an embodiment, the kinase is EGFR. In another embodiment, the kinase is HER,

In yet another aspect, provided herein is a method of treating or preventing a kinase-mediated disorder in an individual in need thereof, comprising administering to the individual a therapeutically effective amount of a compound of the present disclosure. In an embodiment, the kinase-mediated disorder is resistant to an EGFR-targeted therapy. In another embodiment, the EGFR-treated therapy is selected from the group consisting of gefitinib, erlotinib, osimertinib, CO-1686, and WZ4002.

In still another aspect, provided herein is a method of inhibiting the activity of EGFR in a subject in need thereof comprising targeting both Cys775 and Cys797 on EGFR. In yet another aspect, provided herein is a method of inhibiting the activity of EGFR in a subject in need thereof comprising administering a compound that targets both Cys775 and Cys797 on EGFR. The compound can simultaneously form two covalent bonds to cysteine 797 and cysteine 775. In an embodiment, the compound is a compound of Formula I, described herein.

In some embodiments, the compounds of the present disclosure are capable of modulating (e.g., inhibiting or decreasing) the activity of EGFR containing one or more mutations. In some embodiments, the mutant EGFR contains one or more mutations selected from T790M, L718Q, L844V, V948R, L858R, 1941R, C797S, and Del. In other embodiments, the mutant EGFR contains a combination of mutations, wherein the combination is selected from Del/L718Q, Del/L844V, Del/T790M, Del/T790M/L718Q, Del/T790M/L844V, L858R/L718Q, L858R/L844V, L858R/T790M, L858R/T790M/1941R, Del/T790M, Del/T790M/C797S, L858R/T790M/C797S, and L858RIT790M/L718Q. In other embodiments, the mutant EGFR contains a combination of mutations, wherein the combination is selected from Del/L844V, L858R/L844V, L858R/T790M, L858R/T790M/1941R, L858R/T790M/C797S, Del/T790M, Del/T790M, Del/T790M/C797S, and L858R/T790M. In other embodiments, the mutant EGFR contains a combination of mutations, wherein the combination is selected from L858R/T790M, L858R/T790M/1941R, L858R/T790M/C797S, Del/T790M, Del/T790M/C797S, and L858R/T790M.

In some embodiments, the compounds of the present disclosure in combination with a second active agent, wherein said second active agent prevents EGFR dimer formation, are capable of modulating (e.g., inhibiting or decreasing) the activity of EGFR containing one or more mutations. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib.

In some embodiments, the compounds of the present disclosure are capable of modulating (e.g., inhibiting or decreasing) the activity of EGFR containing one or more mutations, but do not affect the activity of a wild-type EGFR.

Modulation of EGFR containing one or more mutations, such as those described herein, but not a wild-type EGFR, provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the compounds of the disclosure exhibit greater inhibition of EGFR containing one or more mutations as described herein relative to a wild-type EGFR. In certain embodiments, the compounds of the disclosure exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the compounds of the disclosure exhibit up to 1000-fold greater inhibition of EGFR containing one or more mutations as described herein relative to a wild-type EGFR. In various embodiments, the compounds of the disclosure exhibit up to 10000-fold greater inhibition of EGFR having a combination of mutations described herein (e.g., L858R/T790M, L858R/T790M/1941R, L858R/T790M/C797S, Del/T790M, Del/T790M/C797S, and L858R/T790M) relative to a wild-type EGFR.

In some embodiments, the inhibition of EGFR activity is measured by IC₅₀. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

In some embodiments, the inhibition of EGFR activity is measured by EC₅₀. A compound with a lower EC₅₀ value, as determined under substantially similar conditions, is a more potent inhibitor relative to a compound with a higher EC₅, value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

In some embodiments, the inhibition of EGFR by a compound of the disclosure can be measured via a biochemical assay. By illustrative and non-limiting example, a homogenous time-resolved fluorescence (HTRF) assay may be used to determine inhibition of EGFR activity using conditions and experimental parameters disclosed herein. The HTRF assay may, for example, employ concentrations of substrate (e.g., biotin-Lck-peptide substrate) of about 1 μM: concentrations of EGFR (mutant or WT) from about 0.2 nM to about 40 nM; and concentrations of inhibitor from about 0.000282 μM to about 50 μM. A compound of the disclosure screened under these conditions may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM. In certain embodiments, a compound of the disclosure screened under the above conditions for inhibition of EGFR having a mutation or combination of mutations selected from L858R/T790M, L858R, and T790M may, for example, exhibit an IC₅₀ value from about 1 nM to >1 μM: from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

In some embodiments, the compounds of the disclosure bind to an allosteric site in EGFR. In some embodiments, the compounds of the disclosure interact with at least one amino acid residue of epidermal growth factor receptor (EGFR) selected from Lys745, Leu788, and Ala743. In other embodiments, the compounds of the disclosure interact with at least one amino acid residue of epidermal growth factor receptor (EGFR) selected from Cys755, Leu777, Phe856, and Asp855. In other embodiments, the compounds of the disclosure interact with at least one amino acid residue of epidermal growth factor receptor (EGFR) selected from Met766, Ile759, Glu782, and Ala763. In other embodiments, the compounds of the disclosure interact with at least one amino acid residue of epidermal growth factor receptor (EGFR) selected from Lys745, Leu788, and Ala 743; at least one amino acid residue of epidermal growth factor receptor (EGFR) selected from Cys755, Leu777, Phe856, and Asp855; and at least one amino acid residue of epidermal growth factor receptor (EGFR) selected from Met766, Ile759, Glu762, and Ala763. In other embodiments, the compounds of the disclosure do not interact with any of the amino acid residues of epidermal growth factor receptor (EGFR) selected from Met793, Gly796, and Cys797.

An EGFR sensitizing mutation comprises without limitation L858R, G719S, G719C, G719A, L861Q, a deletion in exon 19 and/or an insertion in exon 20. A drug-resistant EGFR mutant can have without limitation a drug resistance mutation comprising T790M, T854A, L718Q, C797S, or D761Y.

The selectivity between wild-type EGFR and EGFR containing one or more mutations as described herein can also be measured using cellular proliferation assays where cell proliferation is dependent on kinase activity. For example, murine Ba/F3 cells transfected with a suitable version of wild-type EGFR (such as VIll; containing a WT EGFR kinase domain), or Ba/F3 cells transfected with L858R/T790M, Del/T790M/L718Q, L858R/T790M/L718Q, L858R/T790M/C797S, Del/T790M/C797S, L858R/T790M/1941R, or Exon 19 deletion/T790M can be used. Proliferation assays are performed at a range of inhibitor concentrations (10 μM, 3 μM, 1.1 μM, 330 nM, 110 nM, 33 nM, 11 nM, 3 nM, l nM) and an EC₅₀ is calculated.

In still another aspect, the disclosure provides a method of inhibiting epidermal growth factor receptor (EGFR), the method comprising administering to a subject in need thereof an effective amount of a compound disclosed herein, or a pharmaceutically acceptable salt thereof. In some embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In another aspect, provided herein is a method of treating or preventing a disease, the method comprising administering to a subject in need thereof an effective amount of a compound disclosed herein, or a pharmaceutically acceptable salt thereof. In some embodiments, the disease is mediated by a kinase. In further embodiments, the kinase comprises a mutated cysteine residue. In further embodiments, the mutated cysteine residue is located in or near the position equivalent to Cys797 in EGFR, including such positions in Jak3, Blk, Bmx, Btk, HER2 (ErbB2), HER4 (ErbB4), Itk, Tec, and Txk. In some embodiments, the method further comprises administering a second active agent, wherein said second active agent prevents dimer formation of the kinase. In some embodiments, the second active agent that prevents kinase dimer formation is an antibody. In further embodiments, the second active agent prevents EGFR dimer formation. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disease is mediated by EGFR (e.g., EGFR plays a role in the initiation or development of the disease). In some embodiments, the disease is mediated by a Her-kinase. In further embodiments, the Her-kinase is HER1, HER2, or HER4.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the disease is resistant to a known EGFR inhibitor, including but not limited to, gefitinib, erlotinib, osimertinib, CO-1686, or WZ4002. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In yet another aspect, provided herein is a method of treating a kinase-mediated disorder comprising administering to a subject in need thereof an effective amount of a compound disclosed herein, or a pharmaceutically acceptable salt thereof. In some embodiments, the compound is an inhibitor of HER1, HER2, or HER4. In other embodiments, the subject is administered an additional therapeutic agent. In other embodiments, the compound and the additional therapeutic agent are administered simultaneously or sequentially.

In another aspect, the disclosure provides a method of treating a kinase mediated disorder, the method comprising administering to a subject in need thereof an effective amount of a compound disclosed herein, or a pharmaceutically acceptable salt thereof, and a second active agent, wherein said second active agent prevents EGFR dimer formation. In some embodiments, the compound is an inhibitor of HER1, HER2, or HER4. In other embodiments, the subject is administered an additional therapeutic agent. In other embodiments, the compound, the second active agent that prevents EGFR dimer formation, and the additional therapeutic agent are administered simultaneously or sequentially. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In other embodiments, the disease is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the EGFR activation is selected from mutation of EGFR, amplification of EGFR, expression of EGFR, and ligand mediated activation of EGFR.

In further embodiments, the mutation of EGFR is selected from G719S, G719C, G719A, L858R, L861Q, an exon 19 deletion mutation, and an exon 20 insertion mutation.

In still another aspect, provided herein is a method of treating cancer in a subject, wherein the subject is identified as being in need of EGFR inhibition for the treatment of cancer, comprising administering to the subject an effective amount of a compound disclosed herein, or a pharmaceutically acceptable salt thereof.

In certain embodiments, the subject identified as being in need of EGFR inhibition is resistant to a known EGFR inhibitor, including but not limited to, gefitinib, erlotinib, osirnertinib, CO-1686, or WZ4002. In certain embodiments, a diagnostic test is performed to determine if the subject has an activating mutation in EGFR. In certain embodiments, a diagnostic test is performed to determine if the subject has an EGFR harboring an activating mutation and/or a drug resistance mutation. Activating mutations comprise without limitation L858R, G719S, G719C, G719A, L718Q, L861 Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In an aspect, provided herein is a method of preventing resistance to a known EGFR inhibitor (including but not limited to gefitinib, erlotinib, osimertinib, CO-1686, or WZ4002) in a subject, comprising administering to a subject in need thereof an effective amount of a compound disclosed herein, or a pharmaceutically acceptable salt thereof.

In another aspect, provided herein is a method of preventing resistance to a known EGFR inhibitor (including but not limited to gefitinib, erlotinib, osimertinib, CO-1686, or WZ4002) in a disease, comprising administering to a subject in need thereof an effective amount of a compound disclosed herein, or a pharmaceutically acceptable salt thereof, and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which EGFR plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof. In other embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The activity of the compounds and compositions of the present disclosure as EGFR kinase inhibitors may be assayed in vitro, in vivo, or in a cell line. In vitro assays include assays that determine inhibition of either the kinase activity or ATPase activity of the activated kinase. Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this disclosure as an inhibitor of various kinases are set forth in the Examples below.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent, wherein said second active agent prevents EGFR dimer formation. For any of the above uses, the required dosage will vary depending on the mode of administration, the particular condition to be treated and the effect desired.

In other embodiments, the compound and the second active agent that prevents EGFR dimer formation are administered simultaneously or sequentially.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, an agent that prevents EGFR dimer formation, chemotherapeutic agents or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer. In certain embodiments, the kit further comprises components for performing a test to determine whether a subject has activating and/or drug resistance mutations in EGFR.

In another aspect, the disclosure provides a kit comprising a compound capable of inhibiting EGFR activity selected from a compound disclosed herein, or a pharmaceutically acceptable salt thereof.

In another aspect, the disclosure provides a kit comprising a compound capable of inhibiting kinase activity selected from one or more compounds of disclosed herein, or pharmaceutically acceptable salts thereof; a second active agent, wherein said second active agent prevents EGFR dimer formation; and instructions for use in treating cancer. In certain embodiments, the kit further comprises components for performing a test to determine whether a subject has activating and/or drug resistance mutations in EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In another aspect, the disclosure provides a kit comprising a compound capable of inhibiting EGFR activity selected from a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Abbreviations

ACN acetonitrile
AcOH acetic acid
DCE dichloroethane
DCM dichloromethane
DIEA/DIPEA diisopropylethylamine
EtOAc ethyl acetate
EtOH ethanol
LAH lithium aluminum hydride
m-CPBA meta-chloroperoxybenzoic acid
MeOH methanol
TEA triethylamine
TFA trifluoroacetic acid
THE tetrahydrofuran
TLC thin layer chromatography

### Example 1: Synthetic Procedures

### Procedure for Preparation of Compound 001

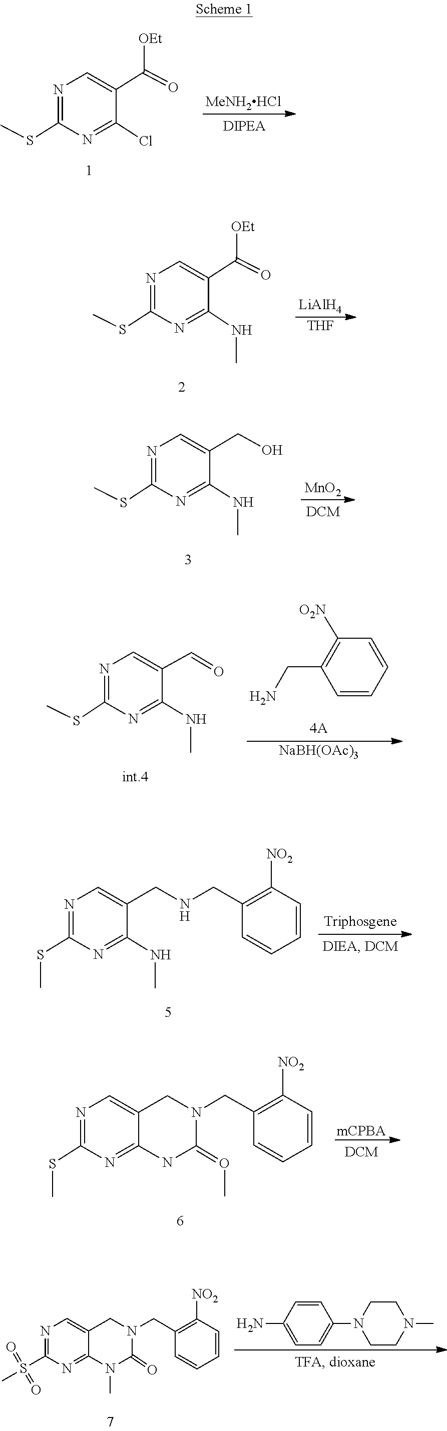

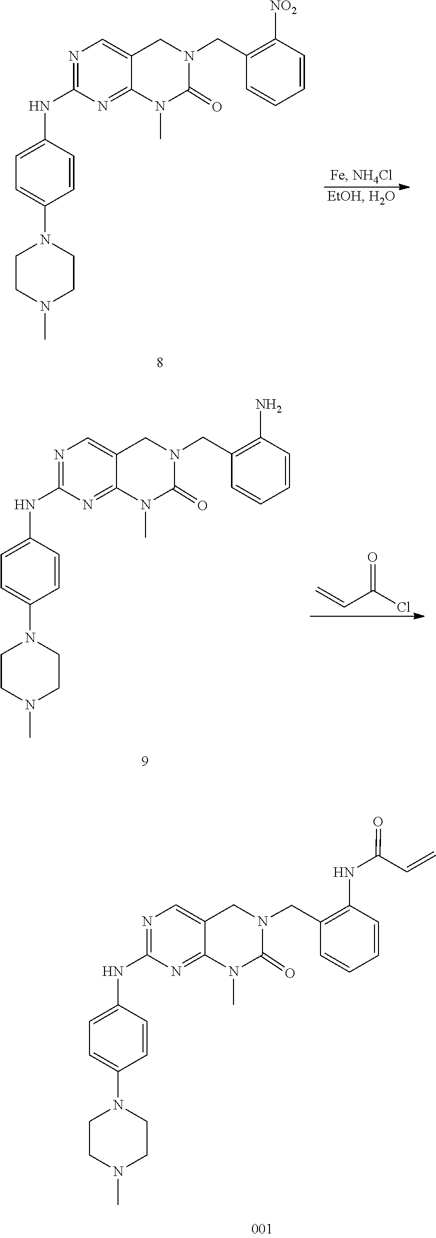

To a solution of ethyl 4-chloro-2-methylsulfanyl-pyrimidine-5-carboxylate (10 g, 42.98 mmol, 1 eq) in THE (200 mL) was added TEA (10.00 g, 98.85 mmol, 13.76 mL, 2.3 eq) and methenamine hydrochloride (6.67 g, 98.85 mmol, 2.3 eq) at 0° C. The mixture was stirred at 0° C. for 1 hr and at 25° C. for 12 hr. LCMS showed complete consumption of the reactant and formation of the desired product mass. The reaction mixture was concentrated under reduced pressure. The residue was diluted with H₂O 100 mL and extracted with EtOAc (100 mL×3). The combined organic layers were washed with brine (100 mL×3), dried over Na₂SO₄, filtered and concentrated under reduced pressure to give ethyl 4-(methylamino)-2-methylsulfanyl-pyrimidine-5-carboxylate (9.4 g) as a white solid.

The suspension of LAH (2.35 g, 62.04 mmol, 1.5 eq) in THE (40 mL) was cooled at 0° C. and to this the solution of ethyl 4-(methylamino)-2-methylsulfanyl-pyrimidine-5-carboxylate (9.4 g, 41.36 mmol, 1 eq) in THE (20 mL) was added dropwise under argon and allowed to stir at 0° C. for 30 min. TLC indicated one major new spot was detected. The reaction was quenched by addition of saturated MgSO₄ solution (20 mL). The mixture and filtered and the filtrate concentrated under reduced pressure to give [4-(methylamino)-2-methylsulfanyl-pyrimidin-5-yl]methanol (5.5 g, crude) as a yellow solid.

To a solution of [4-(methylamino)-2-methylsulfanyl-pyrimidin-5-yl]methanol (3.5 g, 18.89 mmol, 1 eq) in DCM (50 mL) was added MnO₂ (9.86 g, 113.36 mmol, 6 eq). The mixture was stirred at 40° C. for 12 hr. LC-MS showed the desired compound was detected. The reaction mixture was filtered and the filtrate was concentrated under reduced pressure to give a residue, the residue was purified by flash silica gel chromatography (ISCO®; 20 g SepaFlash® Silica Flash Column, Eluent of 5-50% Ethyl acetate/Petroleum ether gradient @ 80 mL/min) to give 4-(methylamino)-2-methylsulfanyl-pyrimidine-5-carbaldehyde (3 g, 16.37 mmol, 87% yield) as a yellow solid.

To a mixture of (2-nitrophenyl)methanamine hydrochloride (2 g, 10.60 mmol, 1 eq) in DCE (30 mL) was added TEA to adjust pH-=8, 4-(methylamino)-2-methylsulfanyl-pyrimidine-5-carbaldehyde (2.91 g, 15.91 mmol, 1.5 eq) and AcOH was added to adjust pH-5 and the mixture was stirred at 25° C. for 2 hr. NaBH(OAc)₃ (6.74 g, 31.81 mmol, 3 eq) was added to the reaction. The mixture was stirred at 25° C. for 12 hr. LCMS showed the desired compound was detected. The reaction mixture was partitioned between H₂O (30 mL) and DCM (30 mL). The water phase was separated, extracted with DCM (30 mL×3), dried over Na₂SO₄, filtered and concentrated under reduced pressure to give a residue. The residue was purified by flash silica gel chromatography (ISCO®; 20 g SepaFlash® Silica Flash Column, Eluent of 0-65% Ethyl acetate/Petroleum ether gradient @ 80 mL/min) to give N-methyl-2-(methylthio)-5-(((2-nitrobenzyl)amino)methyl)pyrimidin-4-amine (2.65 g, 8.30 mmol, 78.25% yield) as a yellow oil.

To a solution of N-methyl-2-(methylthio)-5-(((2-nitrobenzyl)amino)methyl)pyrimidin-4-amine (2.65 g, 8.30 mmol, 1 eq) in DCM (30 mL) was added DIEA (3.22 g, 24.89 mmol, 4.34 mL, 3 eq) and bis(trichloromethyl) carbonate (984.88 mg, 3.32 mmol, 0.4 eq) in DCM (10 mL) at 0° C. The mixture was stirred at 25° C. for 2 hr. LCMS showed complete consumption of the reactant and formation of the desired product mass. The reaction mixture was partitioned between NaHCO₃ 50 mL and DCM 50 mL. The water phase was separated, extracted with DCM (50 mL×3), dried over Na₂SO₄, filtered and concentrated under reduced pressure to give a residue. The residue was purified by flash silica gel chromatography (ISCO®; 20 g SepaFlash® Silica Flash Column, Eluent of 0-100% Ethyl acetate/Petroleum ether gradient @80 mL/min) to give 1-methyl-7-(methylthio)-S-(2-nitrobenzyl)-3,4-dihydropyrimido[4,5-d]pyrimidin-2(1H)-one (700 mg, 2.03 mmol, 24.43% yield) as a yellow solid.

A solution of m-CPBA (1.53 g, 7.53 mmol, 85% purity, 4 eq) in DCM (10 mL) was added to 1-methyl-7-(methylthio)-S-(2-nitrobenzyl)-3,4-dihydropyrimido[4,5-d]pyrimidin-2(1 H)-one (650 mg, 1.88 mmol, 1 eq) in DCM (10 mL) at 0° C. The mixture was stirred at 25° C. for 12 hr. LCMS showed complete consumption of the reactant and formation of the desired product mass. The residue was diluted with Na₂SO₃ (10%) 14 mL and extracted with DCM (10 mL×3). The combined organic layers were washed with NaHCO₃ (10 mL×3), dried over Na₂SO₄, filtered and concentrated under reduced pressure to give a residue. The crude material was suspended in ethanol (2 mL) and stirred at room temperature for 30 min. The solid was filtered, washed with ethanol (2 mL) and dried. The residue was purified by flash silica gel chromatography (ISCO®; 12 g SepaFlash® Silica Flash Column, Eluent of 0-100% Ethyl acetate/Petroleum ethergradient @ 80 mL/min) to give 1-methyl-7-(methylsulfonyl)-S-(2-nitrobenzyl)-3,4-dihydropyrimido[4,5-d]pyrimidin-2(1H)-one (450 mg, 1.19 mmol, 63.36% yield) as yellow gum.

To a solution of 1-methyl-7-(methylsulfonyl)-S-(2-nitrobenzyl)-3,4-dihydropyrimido[4,5-d]pyrimidin-2(1H)-one (160 mg, 423.98 umol, 1 eq) in dioxane (5 mL) was added TFA (72.52 mg, 635.97 umol, 47.09 uL, 1.5 eq) and 4-(4-methylpiperazin-1-yl)aniline (121.64 mg, 635.97 umol, 1.5 eq). The mixture was stirred at 120° C. for 12 hr. LCMS showed the desired compound was detected. The reaction mixture was concentrated under reduced pressure. The residue was diluted with NaHCO₃ 10 mL and extracted with EtOAc (10 mL×3). The combined organic layers were washed with brine (10 mL×3), dried over Na₂SO₄, filtered and concentrated under reduced pressure to give a residue. The residue was purified by prep-TLC (SiO₂, DCM: MeOH=10:1) to give 1-methyl-7-((4-(4-methylpiperazin-1-yl)phenyl)amino)-S-(2-nitrobenzyl)-3,4-dihydropyrimido[4,5-d]pyrimidin-2(1H)-one (180 mg, 305.81 umol, 72.13% yield, 83% purity) as a yellow solid.

To a solution of 1-methyl-7-((4-(4-methylpiperazin-1-yl)phenyl)amino)-S-(2-nitrobenzyl)-3,4-dihydropyrimido[4,5-d]pyrimidin-2(1H)-one (180 mg, 368.44 umol, 1 eq) in EtOH (6 mL) and H₂O (2 mL) was added NH₄Cl (39.42 mg, 736.89 umol, 2 eq) and stirred at 25° C. for 5 min. Then the mixture was heated to 90° C. Fe (82.30 mg, 1.47 mmol, 4 eq) was added to the mixture. The mixture was stirred at 90° C. for 2 hr. LCMS showed the desired compound was detected. The reaction mixture was filtered and the filtrate dried in vacuum to give a residue. The residue was purified by prep-TLC (SiO₂, Dichloromethane: Methanol=10:1) to give 3-(2-aminobenzyl)-1-methyl-7-((4-(4-methylpiperazin-1-yl)phenyl)amino)-3,4-dihydropyrimido[4,5-d]pyrimidin-2(1H)-one (110 mg, 239.9 umol, 65% yield) as a yellow solid.

To a solution of 3-(2-aminobenzyl)-1-methyl-7-((4-(4-methylpiperazin-1-yl)phenyl)amino)-3,4-dihydropyrimido[4,5-d]pyrimidin-2(1H)-one (50 mg, 109.04 umol, 1 eq) and DIPEA (42.3 mg, 327.11 umol, 54.1 uL, 3 eq) in DMF (1 mL) was added and prop-2-enoyl chloride (9.87 mg, 109.04 umol, 8.9 uL, 1 eq) at 0° C. The mixture was stirred at 0° C. for 90 min. LCMS showed the desired compound was detected. The reaction mixture was concentrated under reduced pressure to give a residue. The residue was purified by prep-HPLC (FA condition, column: Phenomenex Luna C18 75*30 mm*3 um; mobile phase: [water(FA)-ACN]; B %: 1%-30%, 8 min) to give N-(2-((1-methyl-7-((4-(4-methylpiperazin-1-yl)phenyl)amino)-2-oxo-1,4-dihydropyrimido[4,5-d]pyrimidin-S(2H)-yl)methyl)phenyl)acrylamide (11 mg, 18.54 umol, 17.01% yield, 94.166% purity, FA) as a white solid. MS (ESI): m/z=513.2 [M+H]+

### Procedure for Preparation of Compound 028

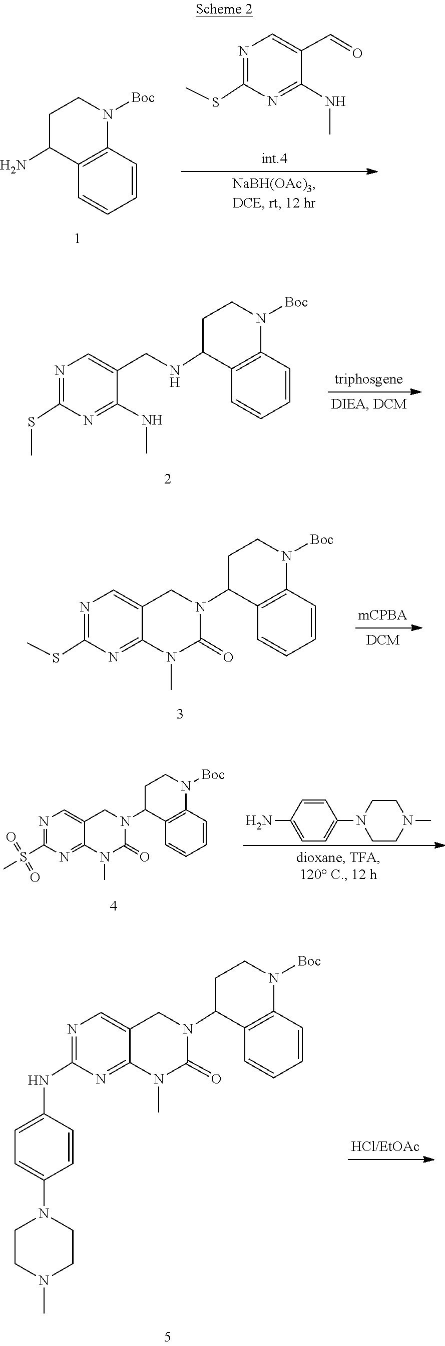

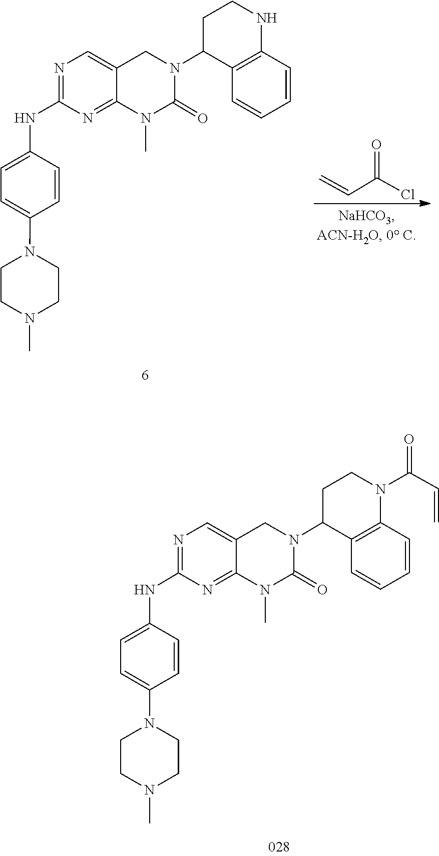

To a solution of tert-butyl 4-amino-3,4-dihydro-2H-quinoline-1-carboxylate (800 mg, 3.22 mmol, 1 eq) in DCE (40 mL) was added 4-(methylamino)-2-methylsulfanyl-pyrimidine-5-carbaldehyde (885.46 mg, 4.83 mmol, 1.5 eq) and AcOH was added to the mixture to adjust pH=5 and the mixture was stirred at 25° C. for 2 hr. NaBH(OAc)₃ (4.10 g, 19.33 mmol, 6 eq) was added to the mixture. The mixture was stirred at 25° C. for 12 hr. LCMS showed consumption of reactant and formation of the desired product mass. The reaction mixture was partitioned between NaHCO₃ (20 mL) and dichloromethane (30 mL). The organic phase was separated, washed with dichloromethane (30 mL×3), dried over Na₂SO₄, filtered and concentrated under reduced pressure to give a residue. The residue was purified by flash silica gel chromatography (ISCO®; 20 g SepaFlash® Silica Flash Column, Eluent of 0-100% Ethyl acetate/Petroleum ether gradient @ 120 mL/min) to give tert-butyl 4-[[4-(methylamino)-2-methylsulfanyl-pyrimidin-5-yl]methylamino]-3,4-dihydro-2H-quinoline-1-carboxylate (860 mg, 2.07 mmol, 64.24% yield) as a white solid.

To a solution of tert-butyl 4-[[4-(methylamino)-2-methylsulfanyl-pyrimidin-5-yl]methylamino]-3,4-dihydro-2H-quinoline-1-carboxylate (860 mg, 2.07 mmol, 1 eq) in DCM (15 mL) was added DIEA (802.4 mg, 6.21 mmol, 1.08 mL, 3 eq) and bis(trichloromethyl) carbonate (307.07 mg, 1.03 mmol, 0.5 eq) at 0° C. The mixture was stirred at 25° C. for 12 hr. LCMS showed consumption of reactant and formation of the desired product mass. The reaction mixture was partitioned between NaHCO₃ (20 mL) and dichloromethane (30 mL). The water phase was separated, extracted with dichloromethane (10 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @ 100 mL/min) to give tert-butyl 4-(1-methyl-7-methylsulfanyl-2-oxo-4H-pyrimido[4,5-d]pyrimidin-S-yl)-3,4-dihydro-2H-quinoline-1-carboxylate (900 mg, 2.04 mmol, 98.49% yield) as a white solid.

To a solution of tert-butyl 4-(1-methyl-7-methylsulfanyl-2-oxo-4H-pyrimido[4,5-d]pyrimidin-S-yl)-3,4-dihydro-2H-quinoline-1-carboxylate (780 mg, 1.77 mmol, 1 eq) in DCM (10 mL) was added m-CPBA (681.41 mg, 3.36 mmol, 85% purity, 1.9 eq) at 0° C. The reaction mixture was stirred at 25° C. for 12 hr. LCMS showed consumption of reactant and formation of the desired product mass. The residue was diluted with Na₂SO₃ (10%) 40 mL and extracted with dichloromethane (20 mL×3). The combined organic layers were washed with NaHCO₃ (20 mL×3), dried over Na₂SO₄, filtered and concentrated under reduced pressure to give tert-butyl 4-(1-methyl-7-methylsulfonyl-2-oxo-4H-pyrimido[4,5-d]pyrimidin-S-yl)-3,4-dihydro-2H-quinoline-1-carboxylate (770 mg, crude) as a white solid.

To a solution of tert-butyl 4-(1-methyl-7-methylsulfonyl-2-oxo-4H-pyrimido[4,5-d]pyrimidin-S-yl)-3,4-dihydro-2H-quinoline-1-carboxylate (300 mg, 633.52 umol, 1 eq) in dioxane (3 mL) was added 4-(4-methylpiperazin-1-yl)aniline (181.76 mg, 950.28 umol, 1.5 eq) and TFA (108.35 mg, 950.28 umol, 70.36 uL, 1.5 eq). The reaction mixture was stirred at 120° C. for 12 hr. LCMS showed consumption of reactant and formation of the desired product mass. The reaction mixture was filtered and concentrated under reduced pressure to give a residue. The residue was purified by prep-TLC (SiO₂, Dichloromethane: Methanol=10:1) to give tert-butyl 4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (175 mg, 299.29 umol, 47.24% yield) as a brown solid.

To a solution of tert-butyl 4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (125 mg, 213.78 umol, 1 eq) in HCl/EtOAc (2 mL, 4M). The mixture was stirred at 25° C. for 1 hr. LCMS showed consumption of reactant and formation of the desired product mass. The reaction mixture was concentrated under reduced pressure to remove solvent, the residue was basified by Et₃N and purified by prep-TLC [SiO₂, Dichloromethane: Methanol (1% NH₃)=10:1] to give 1-methyl-7-[4-(4-methylpiperazin-1-yl)anilino]-S-(1,2,3,4-tetrahydroquinolin-4-yl)-4H-pyrimido[4,5-d]pyrimidin-2-one (100 mg, 206.36 umol) as a yellow solid.

To a solution of 1-methyl-7-[4-(4-methylpiperazin-1-yl)anilino]-S-(1,2,3,4-tetrahydroquinolin-4-yl)-4H-pyrimido [4,5-d]pyrimidin-2-one (50 mg, 103.18 umol, 1 eq) and NaHCO₃ (26 mg, 309.54 umol, 3 eq) in ACN-H₂O (3 mL, 4:1 v/v) was added prop-2-enoyl chloride (9.3 mg, 103.18 umol, 8.4 uL, 1 eq) at 0° C. The mixture was stirred at 0° C. for 1 hr. LCMS showed consumption of reactant and formation of the desired product mass. The reaction mixture was concentrated under reduced pressure to give a residue. The residue was purified by prep-HPLC (FA condition, column: Phenomenex Luna C18 75*30 mm*3 um; mobile phase: [water(FA)-ACN]; B %: 10%-40%, 8 min) to give 1-methyl-7-[4-(4-methylpiperazin-1-yl)anilino]-S-(1-prop-2-enoyl-3,4-dihydro-2H-quinolin-4-yl)-4H-pyrimido[4,5-d]pyrimidin-2-one (15.35 mg, 27.86 umol, 27.00% yield, 97.75% purity) as a yellow solid.

### Procedure for Preparation of Compound 047

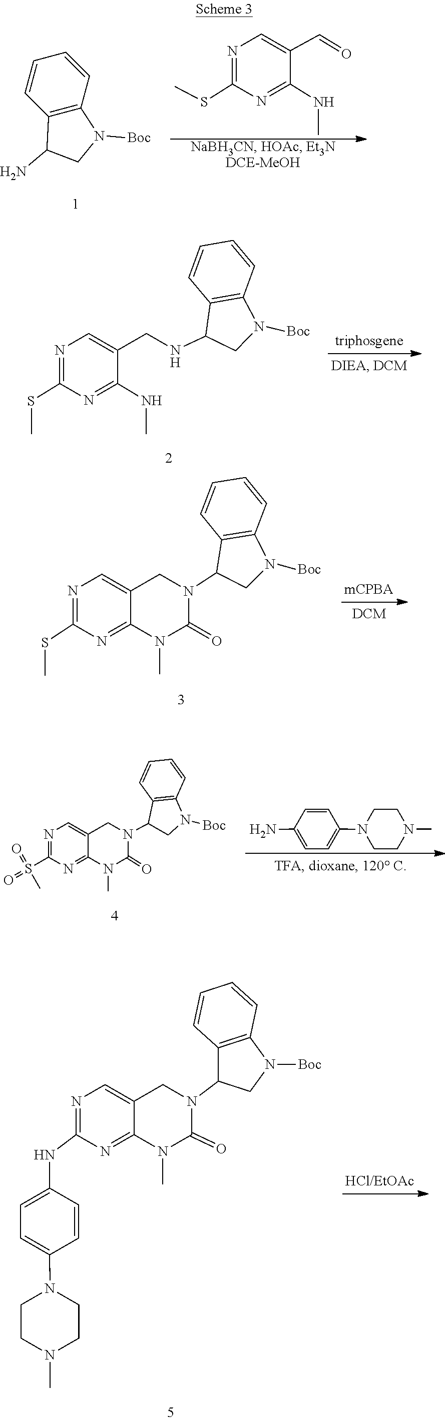

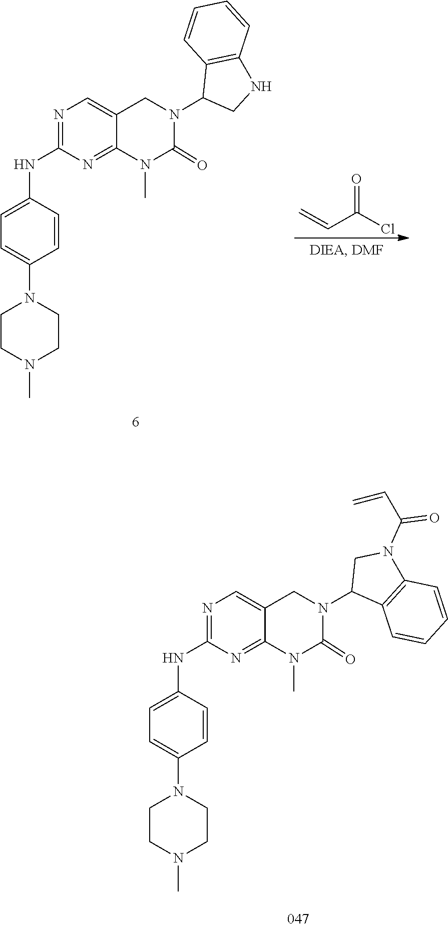

To a solution of 4-(methylamino)-2-methylsulfanyl-pyrimidine-5-carbaldehyde (355.5 mg, 1.9 mmol, 1 eq) in MeOH (20 mL) was added DIEA (626.8 mg, 4.8 mmol, 844.8 uL, 2.5 eq), tert-butyl 3-aminoindoline-1-carboxylate (500 mg, 2.1 mmol, 1.1 eq), AcOH (699.0 mg, 11.6 mmol, 665.7 uL, 6 eq) and NaBH₃CN (731.5 mg, 11.6 mmol, 6 eq). The mixture was stirred at 25° C. for 12 hr. LCMS showed desired mass was detected. The reaction mixture was concentrated under reduced pressure to remove solvent. The residue was diluted with NaHCO₃ (100 mL) and extracted with ethyl acetate (100 mL×3). The combined organic layers were washed with aqueous NaCl (100 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @ 100 mL/min) to give tert-butyl 3-[[4-(methylamino)-2-methylsulfanyl-pyrimidin-5-yl]methylamino]indoline-1-carboxylate (600 mg, 1.5 mmol, 77.0% yield) obtained as a white solid.

To a solution of tert-butyl 3-[[4-(methylamino)-2-methylsulfanyl-pyrimidin-5-yl]methylamino]indoline-1-carboxylate (600 mg, 1.5 mmol, 1 eq) in DCM (10 mL) was added DIEA (579.4 mg, 4.5 mmol, 780.8 uL, 3 eq) and bis(trichloromethyl) carbonate (443.4 mg, 1.5 mmol, 1 eq) at 0° C. The mixture was stirred at 25° C. for 12 hr. The reaction was monitored by LCMS which showed the reactant was consumed completely and the desired mass peak was detected. The reaction mixture was partitioned between NaHCO₃ (10 mL) and DCM (10 mL). The water phase was separated, extracted with DCM (10 mL×3), dried over Na₂SO₄, filtered and concentrated under reduced pressure to give tert-butyl 3-(1-methyl-7-methylsulfanyl-2-oxo-4H-pyrimido[4,5-d]pyrimidin-S-yl)indoline-1-carboxylate (800 mg, crude) obtained as a white solid.

To a solution of tert-butyl 3-(1-methyl-7-methylsulfanyl-2-oxo-4H-pyrimido[4,5-d]pyrimidin-S-yl) indoline-1-carboxylate (800 mg, 1.9 mmol, 1 eq) in DCM (5 mL) was added m-CPBA (759.8 mg, 3.7 mmol, 85% purity, 2 eq) at 0° C. The mixture was stirred at 25° C. for 2 hr. The reaction was monitored by LCMS which showed the reactant was consumed completely and the desired mass peak was detected. The residue was diluted with Na₂SO₃ (10%, 20 mL) and extracted with DCM (20 mL×3). The combined organic layers were washed with NaHCO₃ (20 mL×3), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @ 120 mL/min) to give tert-butyl 3-(1-methyl-7-methylsulfonyl-2-oxo-4H-pyrimido[4,5-d]pyrimidin-S-yl)indoline-1-carboxylate (400 mg, 870.5 umol, 46.5% yield) obtained as a white solid.

To a solution of tert-butyl 3-(1-methyl-7-methylsulfonyl-2-oxo-4H-pyrimido[4,5-d]pyrimidin-S-yl)indoline-1-carboxylate (180 mg, 391.7 umol, 1 eq) in dioxane (4 mL) was added TFA (133.9 mg, 1.2 mmol, 87.0 uL, 3 eq) and 4-(4-methylpiperazin-1-yl)aniline (112.4 mg, 587.6 umol, 1.5 eq). The mixture was stirred at 120° C. for 12 hr. The reaction was monitored by LCMS which showed the reactant was consumed completely and the desired mass peak was detected. The reaction mixture was partitioned between NaHCO₃ (10 mL) and DCM (10 mL). The water phase was separated, extracted with DCM (10 mL×3), dried over Na₂SO₄, filtered and concentrated under reduced pressure to give a residue. The residue was purified by prep-TLC (SiO2, DCM: MeOH=10:1) to give tert-butyl 3-[1-methyl-7-[4-(4-methylpiperazin-1-yl)anilino]-2-oxo-4H-pyrimido[4,5-d]pyrimidin-S-yl]indoline-1-carboxylate (100 mg, 175.2 umol, 44.7% yield) obtained as a yellow solid.

To a solution of 3-indolin-S-yl-1-methyl-7-[4-(4-methylpiperazin-1-yl)anilino]-4H-pyrimido[4,5-d]pyrimidin-2-one (40 mg, 85.0 umol, 1 eq) and DIEA (32.9 mg, 255.0 umol, 44.4 uL, 3 eq) in DMF (1 mL) was added prop-2-enoyl chloride (7.7 mg, 85.0 umol, 6.9 uL, 1 eq) at 0° C. The mixture was stirred at 0° C. for 1 hr. The reaction was monitored by LCMS which showed complete consumption of reactant and detection of the desired mass peak. The reaction mixture was filtered. The residue was purified by prep-HPLC (FA condition; column: Phenomenex Luna C18 75*30 mm*3 um; mobile phase: [water(FA)-ACN]; B %: 1%-30%, 8 min) to yield 1-methyl-7-[4-(4-methylpiperazin-1-yl)anilino]-S-(1-prop-2-enoylindolin-3-yl)-4H-pyrimido[4,5-d]pyrimidin-2-one (6 mg, 9.8 umol, 11.5% yield, 93.400% purity, FA) obtained as a white solid. Analytic data HNMR and QC were consistent with the desired product.

### Procedure for Preparation of Compounds 048 and 049

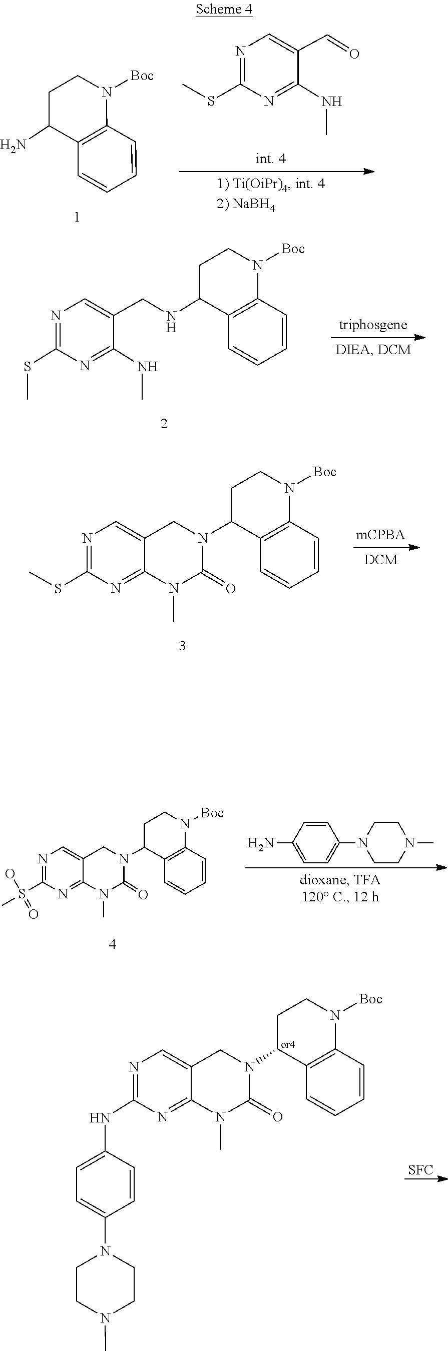

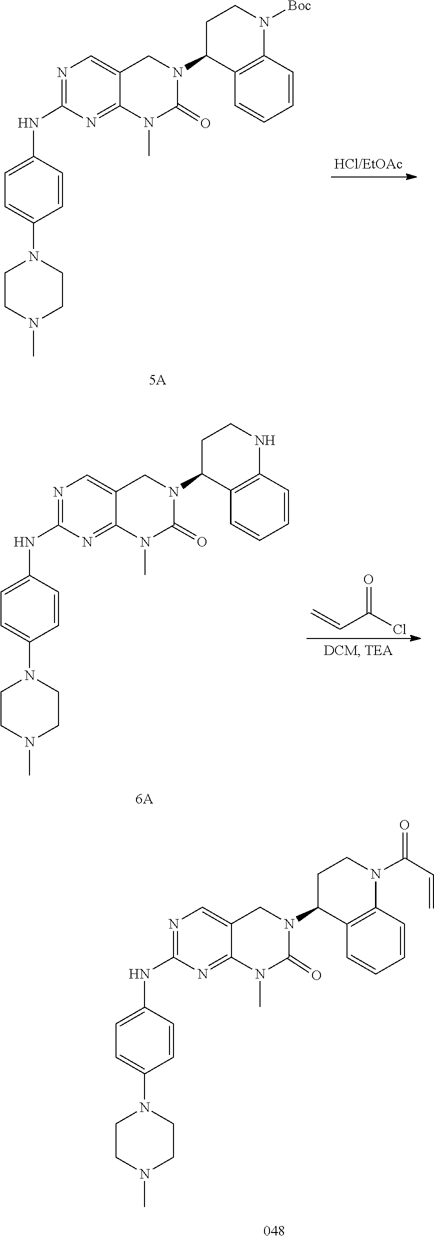

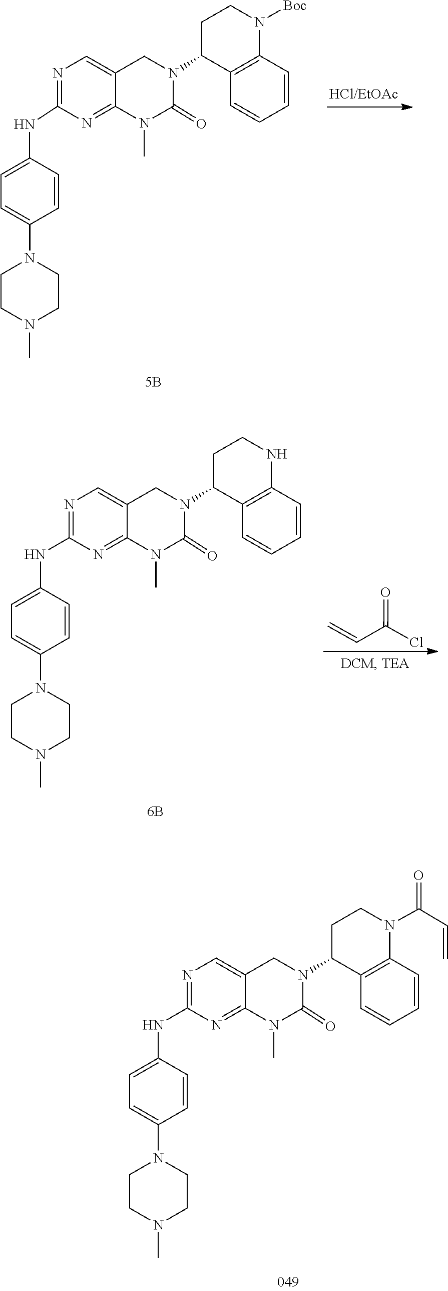

To a solution of 4-(methylamino)-2-methylsulfanyl-pyrimidine-5-carbaldehyde (3 g, 16.37 mmol, 1 eq) in MeOH (100 mL) was added tert-butyl 4-amino-3,4-dihydro-2H-quinoline-1-carboxylate (4.07 g, 16.37 mmol, 1 eq) and tetraisopropoxytitanium (13.96 g, 49.12 mmol, 14.50 mL, 3 eq). The mixture was stirred at 30° C. for 12 hr. Then NaBH₄ (3.72 g, 98.24 mmol, 6 eq) was added to the mixture at 0° C. The mixture was stirred at 25° C. for 3 hr. LC-MS showed the desired compound was detected. The reaction mixture was concentrated under reduced pressure to give a residue. The reaction mixture was quenched by addition NH₄Cl (100 mL) at 0° C. The reaction mixture was extracted with acetate ethyl (100 mL×3). The combined organic layers were washed with brine (30 mL×2), dried over Na₂SO₄, filtered and concentrated under reduced pressure to give a residue. The residue was purified by flash silica gel chromatography (ISCO®; 40 g SepaFlash® Silica Flash Column, Eluent of 0-50% Ethyl acetate/Petroleum ether gradient @ 120 mL/min) to give tert-butyl 4-[[4-(methylamino)-2-methylsulfanyl-pyrimidin-5-yl]methylamino]-3,4-dihydro-2H-quinoline-1-carboxylate (6 g) as a light yellow oil.

To a solution of tert-butyl 4-[[4-(methylamino)-2-methylsulfanyl-pyrimidin-5-yl]methylamino]-3,4-dihydro-2H-quinoline-1-carboxylate (6 g, 14.44 mmol, 1 eq) and DIEA (9.33 g, 72.19 mmol, 12.57 mL, 5 eq) in DCM (100 mL) was added triphosgene (4.28 g, 14.44 mmol, 1 eq) at 0° C. The mixture was stirred at 25° C. for 12 hr. LC-MS showed the desired compound was detected. The reaction mixture was partitioned between NaHCO₃ (100 mL) and dichloromethane (100 mL×2). The organic phase was separated, washed with brine (100 mL×1), dried over Na₂SO₄, filtered and concentrated under reduced pressure to give a residue. The residue was purified by flash silica gel chromatography (ISCO®; 40 g SepaFlash® Silica Flash Column, Eluent of 0-50% Ethyl acetate/Petroleum ether gradient @ 120 mL/min) to give tert-butyl 4-(1-methyl-7-methylsulfanyl-2-oxo-4H-pyrimido[4,5-d]pyrimidin-S-yl)-3,4-dihydro-2H-quinoline-1-carboxylate (5.8 g, 11.71 mmol, 81.13% yield, 89.181% purity) as a light yellow oil.

To a solution of tert-butyl 4-(1-methyl-7-methylsulfanyl-2-oxo-4H-pyrimido[4,5-d]pyrimidin-S-yl)-3,4-dihydro-2H-quinoline-1-carboxylate (3 g, 6.79 mmol, 1 eq) in DCM (50 mL) was added m-CPBA (2.21 g, 10.87 mmol, 85% purity, 1.6 eq) at 0° C. The mixture was stirred at 25° C. for 12 hr. LC-MS showed the desired compound was detected. The residue was diluted with Na₂SO₃ (50 mL) and extracted with dichloromethane (50 mL×3). The combined organic layers were washed with NaHCO₃ (50 mL×3), dried over Na₂SO₄, filtered and concentrated under reduced pressure to give tert-butyl 4-(1-methyl-7-methylsulfonyl-2-oxo-4H-pyrimido[4,5-d]pyrimidin-S-yl)-3,4-dihydro-2H-quinoline-1-carboxylate (3.2 g, crude) as a white solid.

To a solution of tert-butyl 4-(1-methyl-7-methylsulfonyl-2-oxo-4H-pyrimido[4,5-d]pyrimidin-S-yl)-3,4-dihydro-2H-quinoline-1-carboxylate (2 g, 4.22 mmol, 1 eq) in dioxane (20 mL) was added TFA (722.34 mg, 6.34 mmol, 469.05 uL, 1.5 eq) and 4-(4-methylpiperazin-1-yl)aniline (969.40 mg, 5.07 mmol, 1.2 eq). The mixture was stirred at 120° C. for 12 hr. LC-MS showed the desired compound was detected. The residue was purified by prep-HPLC (FA condition, column: Phenomenex luna C18 (250×70 mm, 15 um); mobile phase: [water (FA)-ACN]; B %: 30%-60%, 20 min) to give product 5.

The product was separated by SFC column: DAICEL CHIRALPAK AD(250 mm×50 mm, 10 um); mobile phase: A: CO2 B: EtOH(0.1% IPAm, v/v); B %: 60%-60%, 7 min to give 5A {tert-butyl (4S)-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate, Rt=1.709 min, 500 mg, 855.12 umol, 20.25% yield} and 5B {tert-butyl (4R)-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate, Rt=1.993 min, 530 mg, 906.43 umol, 21.46% yield} as a brown solid.

To a solution of tert-butyl (4S)-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (350 mg, 598.6 umol, 1 eq) in HCl/EtOAc (4 M, 3.5 mL, 23.3 eq) was stirred at 25° C. for 1 hr. LCMS showed desired mass was detected. The reaction mixture was concentrated under reduced pressure to give product 1-methyl-7-[4-(4-methylpiperazin-1-yl)anilino]-S-[(4S)-1,2,3,4-tetrahydroquinolin-4-yl]-4H-pyrimido[4,5-d]pyrimidin-2-one (350 mg, crude) as yellow solid used into the next step without further purification.

To a solution of 1-methyl-7-[4-(4-methylpiperazin-1-yl)anilino]-S-[(4S)-1,2,3,4-tetrahydroquinolin-4-yl]-4H-pyrimido[4,5-d]pyrimidin-2-one (125 mg, 239.9 umol, 1 eq, HCl) and DIEA (93.0 mg, 719.7 umol, 125.3 uL, 3 eq) in DMF (2 mL) was added a DCM solution of prop-2-enoyl chloride (26.1 mg, 287.9 umol, 23.5 uL, 1.2 eq) at 0° C. The reaction was stirred at 0° C. for 1 hr. The reaction was monitored by LCMS which showed complete consumption of reactant and detection of the desired mass peak. The reaction mixture was filtered. The residue was purified by prep-HPLC (FA condition; column: Phenomenex luna C18 100×40 mm×3 um; mobile phase: [water(FA)-ACN]; B %: 1%-40%, 8 min), and then the residue was purified by prep-HPLC (neutral condition; column: Waters Xbridge Prep OBD C18 150×40 mm×10 um; mobile phase: [water(NH₄HCO₃)-ACN]; B %: 20%-50%, 8 min) to yield 1-methyl-7-[4-(4-methylpiperazin-1-yl)anilino]-S-[(4S)-1-prop-2-enoyl-3,4-dihydro-2H-quinolin-4-yl]-4H-pyrimido[4,5-d]pyrimidin-2-one (100% purity, 69 mg) as a white solid.

A mixture of tert-butyl (4R)-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (350 mg, 598.59 umol, 1 eq) in HCl/EtOAc (4 mL, 4 M) was stirred at 25° C. for 0.5 hr. LC-MS showed the desired compound was detected. The reaction mixture was filtered and the filtrate dried in vacuum to give 1-methyl-7-[4-(4-methylpiperazin-1-yl)anilino]-S-[(4R)-1,2,3,4-tetrahydroquinolin-4-yl]-4H-pyrimido[4,5-d]pyrimidin-2-one (350 mg, crude) as a brown solid.

To a solution of 1-methyl-7-[4-(4-methylpiperazin-1-yl)anilino]-S-[(4R)-1,2,3,4-tetrahydroquinolin-4-yl]-4H-pyrimido[4,5-d]pyrimidin-2-one (300 mg, 575.75 umol, 1 eq, HCl) and TEA (174.78 mg, 1.73 mmol, 240.41 uL, 3 eq) in DCM (5 mL) was added a DCM solution of prop-2-enoyl chloride (52.11 mg, 575.75 umol, 46.95 uL, 1 eq). The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 75×30 mm×3 um; mobile phase: [water(FA)-ACN]; B %: 1%-35%, 8 min) and (NH₄HCO₃ condition, column: Phenomenex C18 75×30 mm×3 um; mobile phase: [water(NH₄HCO₃)-ACN]; B %: 20%-55%, 8 min) to give 1-methyl-7-[4-(4-methylpiperazin-1-yl)anilino]-S-[(4R)-1-prop-2-enoyl-3,4-dihydro-2H-quinolin-4-yl]-4H-pyrimido[4,5-d]pyrimidin-2-one (76.10 mg, 139.89 umol, 24.30% yield, 99.016% purity) as a yellow solid.

### Procedure for Preparation of Compound 052

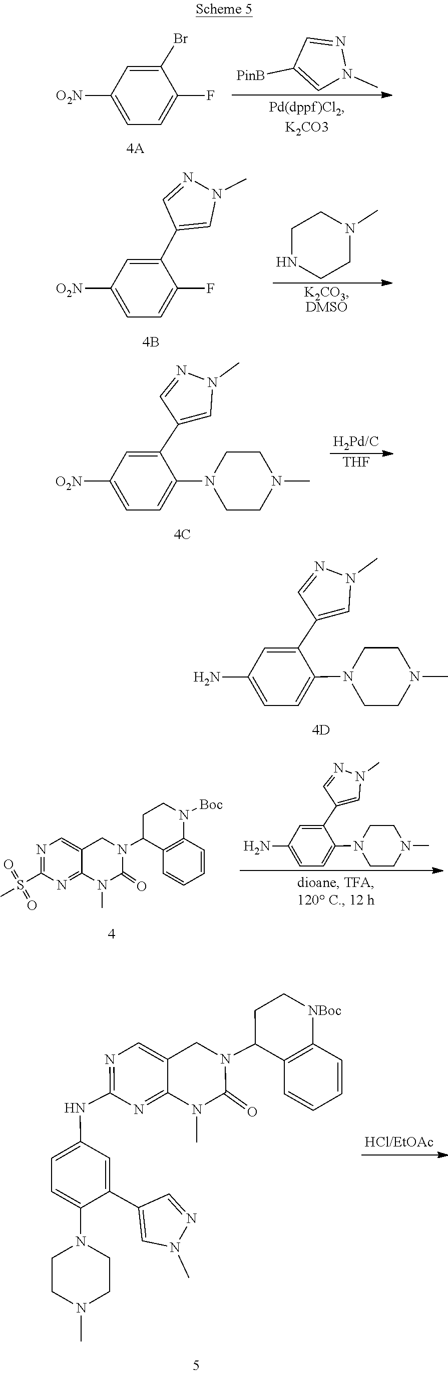

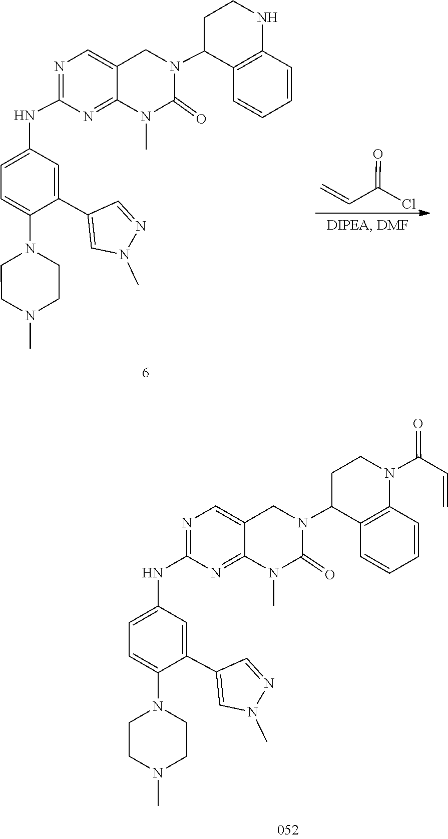

To a solution of 2-bromo-1-fluoro-4-nitro-benzene (1 g, 4.55 mmol, 1 eq) in dioxane (10 mL) and H₂O (1 mL) was added K₂CO₃ (1.88 g, 13.64 mmol, 3 eq), 1-methyl-4-(4,4,5,5-tetramethyl-1,3,2-dioxaborolan-2-yl)pyrazole (1.13 g, 5.45 mmol, 1.2 eq) and Pd(dppf)Cl₂ (332.60 mg, 454.55 umol, 0.1 eq). The mixture was stirred at 90° C. for 12 hr. The reaction mixture was partitioned between H₂O (50 mL) and ethyl acetate (50 mL×3). The organic phase was separated, washed with brine (20 mL×1), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @ 100 mL/min) to give 4-(2-fluoro-5-nitro-phenyl)-1-methyl-pyrazole (1 g, 4.52 mmol, 99.46% yield) as a yellow solid.

To a solution of 4-(2-fluoro-5-nitro-phenyl)-1-methyl-pyrazole (1 g, 4.52 mmol, 1 eq) in DMSO (10 mL) was added K₂CO₃ (1.87 g, 13.56 mmol, 3 eq) and 1-methylpiperazine (543.40 mg, 5.43 mmol, 601.78 uL, 1.2 eq). The mixture was stirred at 80° C. for 12 hr. The reaction mixture was partitioned between H₂O (50 mL) and ethyl acetate (50 mL×3). The organic phase was separated, washed with brine (20 mL×1), dried over Na₂SO₄, filtered and concentrated under reduced pressure to give a residue. The residue was purified by flash silica gel chromatography (ISCO®; 12 g SepaFlash® Silica Flash Column, Eluent of 0-15% Methanol/Dichloromethane ether gradient @ 100 mL/min) to give 1-methyl-4-[2-(1-methylpyrazol-4-yl)-4-nitro-phenyl]piperazine (1.1 g, 3.65 mmol, 80.74% yield) as a yellow solid.

To a solution of Pd/C (500 mg, 10% Pd) in THE (10 mL) was added 1-methyl-4-[2-(1-methylpyrazol-4-yl)-4-nitro-phenyl]piperazine (1.1 g, 3.65 mmol, 1 eq) under H₂ (50 psi) at 25° C. The reaction mixture was stirred at 25° C. for 12 hr. The reaction mixture was filtered and concentrated under reduced pressure to give a residue. The residue was purified by prep-HPLC (NH₃·H₂O+NH₄HCO₃ condition, column: Waters Xbridge Prep OBD C18 150×40 mm×10 um; mobile phase: [water (NH₃·H₂O+NH₄HCO₃)-ACN]; B %: 1%-30%, 8 min) to give 4-(4-methylpiperazin-1-yl)-S-(1-methylpyrazol-4-yl)aniline (120 mg, 442.22 umol, 12.11% yield) as a brown solid.

To a solution of tert-butyl 4-(1-methyl-7-methylsulfonyl-2-oxo-4H-pyrimido[4,5-d]pyrimidin-S-yl)-3,4-dihydro-2H-quinoline-1-carboxylate (150 mg, 316.76 umol, 1 eq) in dioxane (2 mL) was added TFA (54.18 mg, 475.14 umol, 35.18 uL, 1.5 eq) and 4-(4-methylpiperazin-1-yl)-S-(1-methylpyrazol-4-yl)aniline (85.96 mg, 316.76 umol, 1 eq). The mixture was stirred at 120° C. for 12 hr. The reaction mixture was concentrated under reduced pressure to remove solvent. The residue was purified by prep-TLC (SiO2, Dichloromethane: Methanol=10:1) to give tert-butyl 4-[1-methyl-7-[4-(4-methylpiperazin-1-yl)-S-(1-methylpyrazol-4-yl)anilino]-2-oxo-4H-pyrimido[4,5-d]pyrimidin-S-yl]-3,4-dihydro-2H-quinoline-1-carboxylate (200 mg) as a yellow solid.

To a solution of tert-butyl 4-[1-methyl-7-[4-(4-methylpiperazin-1-yl)-S-(1-methylpyrazol-4-yl)anilino]-2-oxo-4H-pyrimido[4,5-d]pyrimidin-S-yl]-3,4-dihydro-2H-quinoline-1-carboxylate (100 mg, 150.42 umol, 1 eq) in HCl/EtOAc (1 mL, 4 M). The mixture was stirred at 25° C. for 1 hr. The reaction mixture was concentrated under reduced pressure to remove solvent to give 1-methyl-7-[4-(4-methylpiperazin-1-yl)-S-(1-methylpyrazol-4-yl)anilino]-S-(1,2,3,4-tetrahydroquinolin-4-yl)-4H-pyrimido[4,5-d]pyrimidin-2-one (90 mg, crude, HCl) as a yellow solid.

To a solution of 1-methyl-7-[4-(4-methylpiperazin-1-yl)-S-(1-methylpyrazol-4-yl)anilino]-S-(1,2,3,4-tetrahydroquinolin-4-yl)-4H-pyrimido[4,5-d]pyrimidin-2-one (90 mg, 159.38 umol, 1 eq) and DIEA (61.80 mg, 478.14 umol, 83.28 uL, 3 eq) in DMF (1 mL) was dropwise added a DCM solution of prop-2-enoyl chloride (14.43 mg, 159.38 umol, 13.00 uL, 1 eq) at 0° C. The mixture was stirred at 25° C. for 1 hr. The reaction mixture was concentrated under reduced pressure to give a residue. The residue was purified by prep-HPLC (FA condition, column: Phenomenex Luna C18 200×40 mm×10 um; mobile phase: [water(FA)-ACN]; B %: 1%-30%, 8 min). The residue was purified by prep-TLC (SiO₂, dichloromethane: methanol=5:1) to give 1-methyl-7-[4-(4-methylpiperazin-1-yl)-S-(1-methylpyrazol-4-yl)anilino]-S-(1-prop-2-enoyl-3,4-dihydro-2H-quinolin-4-yl)-4H-pyrimido[4,5-d]pyrimidin-2-one (2.7 mg, 4.13 umol, 2.59% yield, 94.6% purity) as a yellow solid.

### Procedure for Preparation of Compound 057

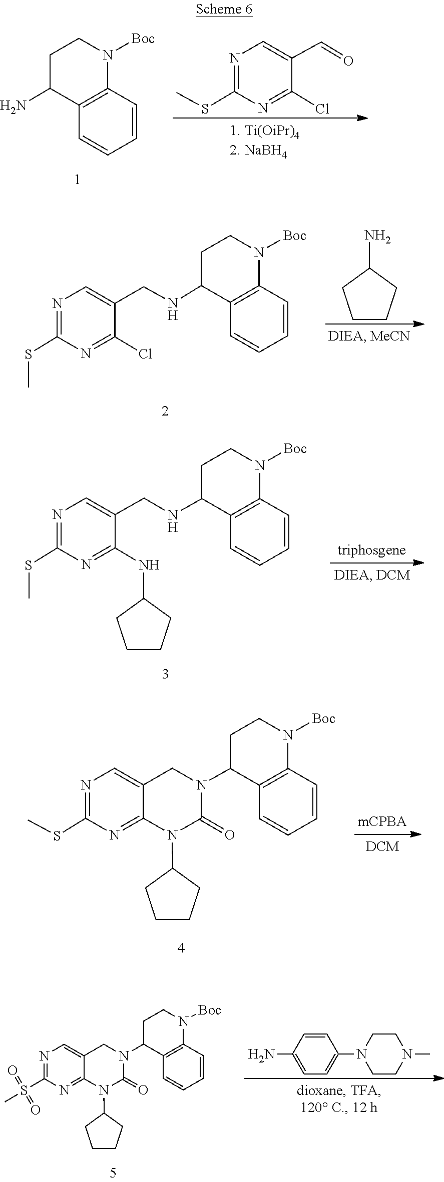

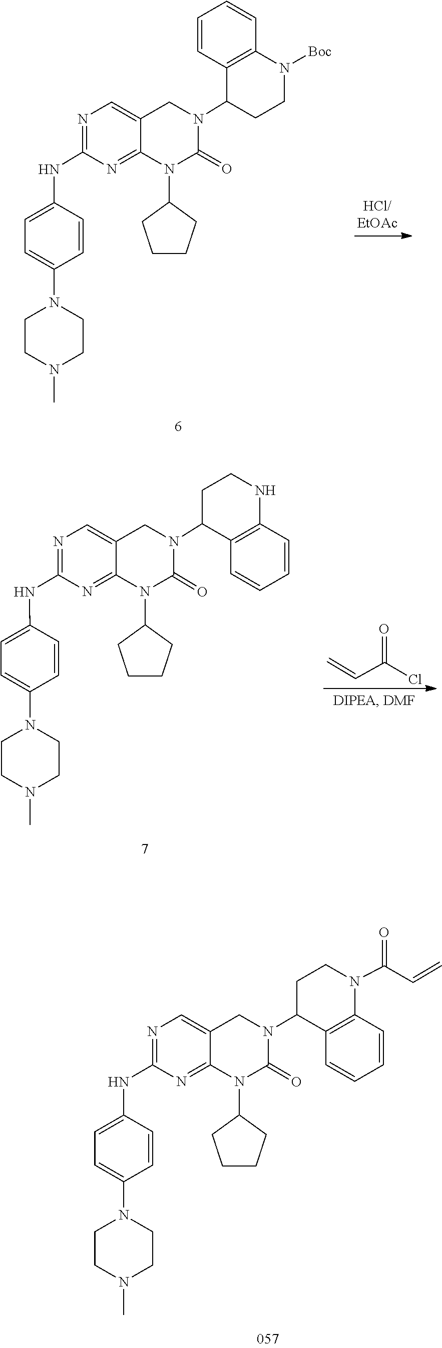

To a solution of tetraisopropoxytitanium (904.0 mg, 3.2 mmol, 938.7 uL, 3 eq) and tert-butyl 4-amino-3,4-dihydro-2H-quinoline-1-carboxylate (263.3 mg, 1.1 mmol, 1 eq) in MeOH (3 mL) was added 4-chloro-2-methylsulfanyl-pyrimidine-5-carbaldehyde (200 mg, 1.1 mmol, 1 eq). The reaction mixture was stirred at 25° C. for 12 hr. NaBH₄ (240.7 mg, 6.4 mmol, 6 eq) was added to the mixture at 0° C. The reaction mixture was stirred at 25° C. for 3 hr. LCMS showed consumption of reactant and formation of the desired product mass. The reaction mixture was concentrated under reduced pressure to give a residue. The reaction mixture was quenched by addition NH₄Cl (10 mL) at 0° C. The reaction mixture was extracted with acetate ethyl (10 mL×3). The combined organic layers were washed with brine (10 mL×3), dried over Na₂SO₄, filtered and concentrated under reduced pressure to give a residue. The residue was purified by prep-TLC (SiO₂, Petroleum ether: Ethyl acetate=3:2) to yield tert-butyl 4-[(4-chloro-2-methylsulfanyl-pyrimidin-5-yl)methylamino]-3,4-dihydro-2H-quinoline-1-carboxylate (250 mg, 593.89 umol, 56.01% yield) as a white solid.

To a solution of tert-butyl 4-[(4-chloro-2-methylsulfanyl-pyrimidin-5-yl)methylamino]-3,4-dihydro-2H-quinoline-1-carboxylate (200 mg, 475.1 umol, 1 eq) in MeCN (2 mL) was added DIEA (184.2 mg, 1.4 mmol, 248.3 uL, 3 eq) and cyclopentanamine (80.9 mg, 950.2 umol, 93.7 uL, 2 eq). The mixture was stirred at 70° C. for 12 hr. The reaction was monitored by LCMS which showed the reactant was consumed completely and the desired mass peak was detected. The reaction mixture was quenched by addition NH₄Cl 15 mL at 0° C., and then extracted with ethyl acetate (15 mL×3). The combined organic layers were washed with brine (20 mL×2), dried over Na₂SO₄, filtered and concentrated under reduced pressure to give tert-butyl 4-[[4-(cyclopentylamino)-2-methylsulfanyl-pyrimidin-5-yl]methylamino]-3,4-dihydro-2H-quinoline-1-carboxylate (200 mg, crude) as a yellow solid.

To a solution of tert-butyl 4-[[4-(cyclopentylamino)-2-methylsulfanyl-pyrimidin-5-yl]methylamino]-3,4-dihydro-2H-quinoline-1-carboxylate (200 mg, 425.9 umol, 1 eq) in DCM (3 mL) was added DIEA (110.1 mg, 851.7 umol, 148.3 uL, 2 eq) and triphosgene (126.4 mg, 425.8 umol, 1 eq) at 0° C. The mixture was stirred at 25° C. for 12 hr. The reaction was monitored by LCMS which showed complete consumption of reactant and detection of the desired mass peak. The reaction mixture was quenched by addition NaHCO₃ (15 mL) at 25° C., and then extracted with ethyl acetate (20 mL×3). The combined organic layers were washed with aqueous NaCl (20 mL×3), dried over Na₂SO₄, filtered and concentrated under reduced pressure to give the crude product tert-butyl 4-(1-cyclopentyl-7-methylsulfanyl-2-oxo-4H-pyrimido[4,5-d]pyrimidin-S-yl)-3,4-dihydro-2H-quinoline-1-carboxylate (210 mg, 423.7 umol, 99.5% yield) as a yellow solid.

A solution of tert-butyl 4-(1-cyclopentyl-7-methylsulfanyl-2-oxo-4H-pyrimido[4,5-d]pyrimidin-S-yl)-3,4-dihydro-2H-quinoline-1-carboxylate (210 mg, 423.70 umol, 1 eq) in DCM (3 mL) was added m-CPBA (172.0 mg, 847.4 umol, 85% purity, 2 eq) at 0° C. The mixture was stirred at 25° C. for 4 hr. The reaction was monitored by LCMS which showed complete consumption of reactant and detection of the desired mass peak. The reaction mixture was quenched by addition Na₂SO₃ (15 mL) at 0° C., and then extracted with DCM (15 mL×3). The combined organic layers were dried over Na₂SO₄, filtered and concentrated under reduced pressure to give a residue. The residue was purified by column chromatography (SiO₂, Petroleum ether/Ethyl acetate=0/1) to yield tert-butyl 4-(1-cyclopentyl-7-methylsulfonyl-2-oxo-4H-pyrimido[4,5-d]pyrimidin-S-yl)-3,4-dihydro-2H-quinoline-1-carboxylate (180 mg, 341.1 umol, 80.5% yield) as yellow oil.

To a solution of 4-(4-methylpiperazin-1-yl)aniline (36.2 mg, 189.5 umol, 1 eq) in dioxane (2 mL) was added tert-butyl 4-(1-cyclopentyl-7-methylsulfonyl-2-oxo-4H-pyrimido[4,5-d]pyrimidin-S-yl)-3,4-dihydro-2H-quinoline-1-carboxylate (100 mg, 189.5 umol, 1 eq) and TFA (32.4 mg, 284.3 umol, 21.0 uL, 1.5 eq). And then the mixture was warmed to 120° C. and stirred for 12 hr. The reaction was monitored by LCMS which showed complete consumption of reactant and detection of the desired product. The residue was purified by prep-HPLC (FA condition; column: Phenomenex Luna C18 75×30 mm×3 um; mobile phase: [water(FA)-ACN]; B %: 20%-50%, 8 min) to give tert-butyl 4-[1-cyclopentyl-7-[4-(4-methylpiperazin-1-yl)anilino]-2-oxo-4H-pyrimido[4,5-d]pyrimidin-S-yl]-3,4-dihydro-2H-quinoline-1-carboxylate (92.43% purity, 120 mg) as a white solid.

A mixture of tert-butyl 4-[1-cyclopentyl-7-[4-(4-methylpiperazin-1-yl)anilino]-2-oxo-4H-pyrimido[4,5-d]pyrimidin-S-yl]-3,4-dihydro-2H-quinoline-1-carboxylate (100 mg, 156.54 umol, 1 eq) in HCl/EtOAc (2 mL, 4M) was stirred at 25° C. for 10 min. The reaction was monitored by LCMS which showed complete consumption of reactant and detection of the desired product. The reaction mixture was concentrated under reduced pressure to give the crude product 1-cyclopentyl-7-[4-(4-methylpiperazin-1-yl)anilino]-S-(1,2,3,4-tetrahydroquinolin-4-yl)-4H-pyrimido[4,5-d]pyrimidin-2-one (100 mg, crude) as yellow solid.

To a solution of 1-cyclopentyl-7-[4-(4-methylpiperazin-1-yl)anilino]-S-(1,2,3,4-tetrahydroquinolin-4-yl)-4H-pyrimido[4,5-d]pyrimidin-2-one (85 mg, 157.8 umol, 1 eq) and DIEA (61.2 mg, 473.4 umol, 82.5 uL, 3 eq) in DMF (1 mL) was dropwise added a DCM solution of prop-2-enoyl chloride (17.1 mg, 189.3 umol, 15.4 uL, 1.2 eq). The mixture was stirred at 0° C. for 1 hr. The reaction was monitored by LCMS which showed complete consumption of reactant and detection of the desired mass peak. The residue was purified by prep-HPLC (FA condition; column: Phenomenex luna C18 100×40 mm×3 um; mobile phase: [water(FA)-ACN]; B %: 15%-45%, 8 min). After prep-HPLC, the residue was further purified by column chromatography by prep-TLC (SiO2, DCM: MeOH=20:1) to yield 1-cyclopentyl-7-[4-(4-methylpiperazin-1-yl)anilino]-S-(1-prop-2-enoyl-3,4-dihydro-2H-quinolin-4-yl)-4H-pyrimido[4,5-d]pyrimidin-2-one (5 mg, 8.3 umol, 5.2% yield, 97.89% purity) as a white solid.

### Procedure for Preparation of Compound 059

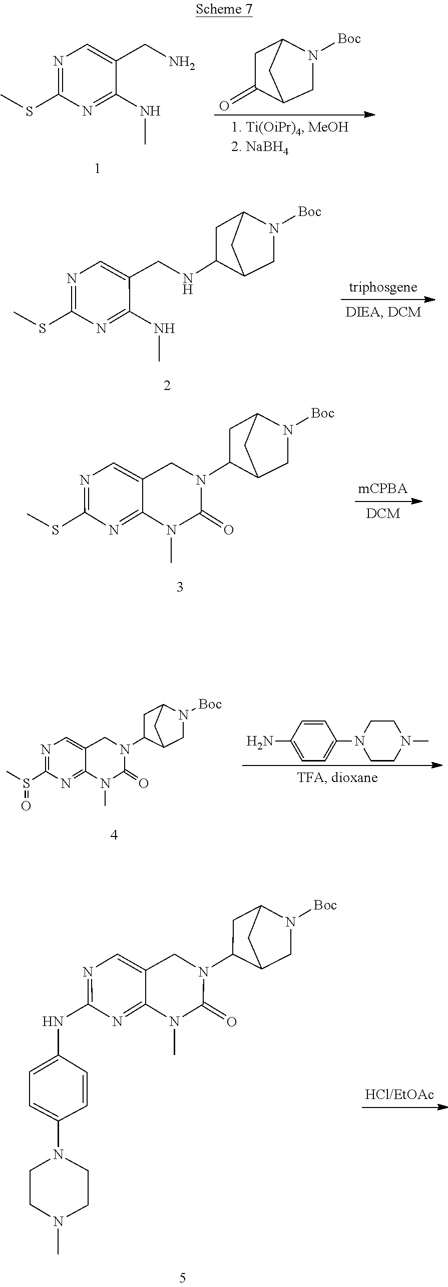

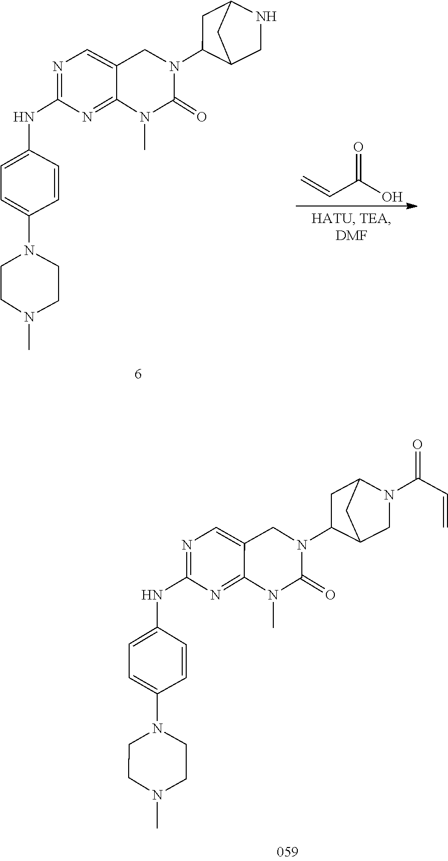

To a solution of 5-(aminomethyl)-N-methyl-2-methylsulfanyl-pyrimidin-4-amine (750.11 mg, 4.07 mmol, 1 eq) and tert-butyl (1S,4S)-5-oxo-2-azabicyclo[2.2.1]heptane-2-carboxylate (860 mg, 4.07 mmol, 1 eq) in MeOH (10 mL) was added tetraisopropoxytitanium (3.47 g, 12.21 mmol, 3.60 mL, 3 eq), stirred at 30° C. for 12 hr. NaBH4 (924.07 mg, 24.43 mmol, 6 eq) was added to the reaction at 0° C. and stirred at 20° C. for 4 hr. LC-MS showed desired compound was detected. The reaction mixture was diluted with NH₄Cl 50 mL and extracted with EtOAc 60 mL (20 mL×3). The combined organic layers were washed with brine 60 mL (30 mL×2), dried over Na₂SO₄, filtered and the filtrate was concentrated under reduced pressure to give a residue. The residue was purified by flash silica gel chromatography (ISCO®; 20 g SepaFlash® Silica Flash Column, Eluent of 0-60% Ethyl acetate/Petroleum ether gradient @ 120 mL/min) to give tert-butyl 5-[[4-(methylamino)-2-methylsulfanyl-pyrimidin-5-yl]methylamino]-2-azabicyclo[2.2.1]heptane-2-carboxylate (1.32 g, 3.14 mmol, 77.12% yield, 90.45% purity) as colorless oil.

To a solution of tert-butyl 5-[[4-(methylamino)-2-methylsulfanyl-pyrimidin-5-yl]methylamino]-2-azabicyclo[2.2.1]heptane-2-carboxylate (1.3 g, 3.43 mmol, 1 eq) and DIEA (1.33 g, 10.28 mmol, 1.79 mL, 3 eq) in DCM (10 mL) was added triphosgene (1.02 g, 3.43 mmol, 1 eq) at 0° C., the reaction was stirred at 25° C. for 12 hr. LC-MS showed desired compound was detected. The residue was diluted with NaHCO₃ 50 mL and extracted with DCM 60 mL (20 mL×3). The combined organic layers were washed with brine 40 mL (20 mL×2), dried over Na₂SO₄, filtered and concentrated under reduced pressure to give tert-butyl 5-(1-methyl-7-methylsulfanyl-2-oxo-4H-pyrimido[4,5-d]pyrimidin-S-yl)-2-azabicyclo[2.2.1]heptane-2-carboxylate (1.62 g, crude) as brown oil.

To a solution of tert-butyl 5-(1-methyl-7-methylsulfanyl-2-oxo-4H-pyrimido[4,5-d]pyrimidin-S-yl)-2-azabicyclo[2.2.1]heptane-2-carboxylate (158.6 mg, 391.11 umol, 1 eq) in DCM (4 mL) was added m-CPBA (142.93 mg, 704.00 umol, 85% purity, 1.8 eq) at 0° C., the reaction was stirred at 25° C. for 12 hr. LC-MS showed desired compound was detected. The residue was diluted with 10% Na₂SO₃ 10 mL and extracted with DCM 30 mL (10 mL×3). The combined organic layers were washed with NaHCO₃ 20 mL (10 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ether gradient @ 120 mL/min) to give tert-butyl 5-(1-methyl-7-methylsulfonyl-2-oxo-4H-pyrimido[4,5-d]pyrimidin-S-yl)-2-azabicyclo[2.2.1]heptane-2-carboxylate (150 mg, 157.37 umol, 40.24% yield, 45.9% purity) as a white solid.

To a solution of tert-butyl 5-(1-methyl-7-methylsulfonyl-2-oxo-4H-pyrimido[4,5-d]pyrimidin-S-yl)-2-azabicyclo[2.2.1]heptane-2-carboxylate (150 mg, 342.85 umol, 1 eq) in dioxane (2 mL) was added 4-(4-methylpiperazin-1-yl)aniline (131.15 mg, 685.70 umol, 2 eq) and TFA (58.64 mg, 514.27 umol, 38.08 uL, 1.5 eq), the reaction was stirred at 120° C. for 12 hr. LC-MS showed desired compound was detected. The reaction mixture was concentrated under reduced pressure to remove solvent. The residue was purified by prep-HPLC (FA condition, column: Phenomenex Luna C18 75*30 mm*3 um; mobile phase: [water(FA)-ACN];B %: 15%-45%, 8 min) to give tert-butyl 5-[1-methyl-7-[4-(4-methylpiperazin-1-yl)anilino]-2-oxo-4H-pyrimido[4,5-d]pyrimidin-S-yl]-2-azabicyclo[2.2.1]heptane-2-carboxylate (104 mg, 189.55 umol, 55.29% yield) as a brown solid.

tert-butyl 5-[1-methyl-7-[4-(4-methylpiperazin-1-yl)anilino]-2-oxo-4H-pyrimido[4,5-d]pyrimidin-S-yl]-2-azabicyclo[2.2.1]heptane-2-carboxylate (100 mg, 182.26 umol, 1 eq) in HCl/EtOAc (1 mL) (4 M) was stirred at 20° C. for 1 hr. LC-MS showed desired compound was detected. The reaction mixture was concentrated under reduced pressure to give 3-(2-azabicyclo[2.2.1]heptan-5-yl)-1-methyl-7-[4-(4-methylpiperazin-1-yl)anilino]-4H-pyrimido[4,5-d]pyrimidin-2-one (105 mg, crude) as a yellow solid.

To a solution of acrylic acid (8.32 mg, 115.46 umol, 7.92 uL, 0.8 eq) in DMF (1 mL) was added HATU (65.85 mg, 173.19 umol, 1.2 eq) and TEA (29.21 mg, 288.65 umol, 40.18 uL, 2 eq). The reaction was stirred at 25° C. for 30 min, 3-(2-azabicyclo[2.2.1]heptan-5-yl)-1-methyl-7-[4-(4-methylpiperazin-1-yl)anilino]-4H-pyrimido[4,5-d]pyrimidin-2-one (70 mg, 144.32 umol, 1 eq, HCl) was added to the mixture and stirred at 25° C. for 12 hr. LC-MS showed desired compound was detected. The reaction mixture was concentrated under reduced pressure to remove solvent. The residue was purified by prep-HPLC (FA condition, column: Phenomenex Luna C18 75*30 mm*3 um;mobile phase: [water(FA)-ACN];B %: 10%-40%, 8 min) to give 1-methyl-7-[4-(4-methylpiperazin-1-yl)anilino]-S-(2-prop-2-enoyl-2-azabicyclo[2.2.1]heptan-5-yl)-4H-pyrimido[4,5-d]pyrimidin-2-one (23.9 mg, 45.95 umol, 31.84% yield, 96.629% purity) as a white solid.

### Procedure for Preparation of Compound 060

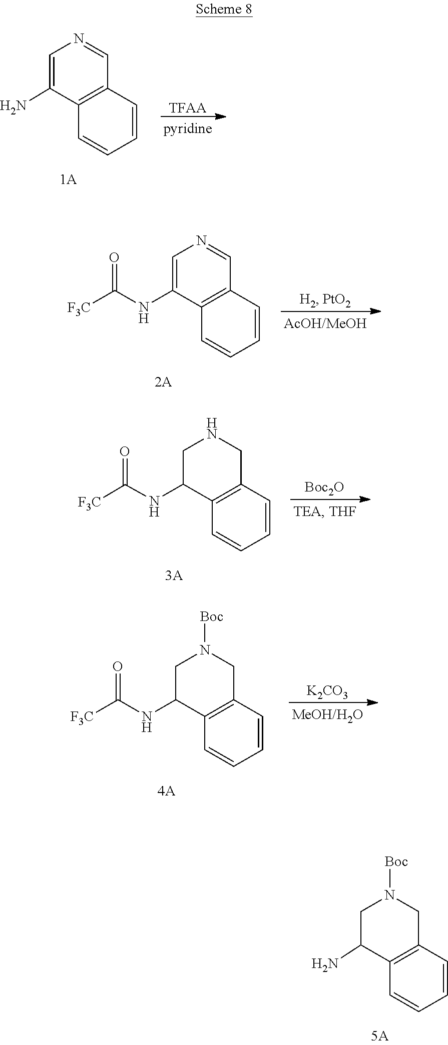

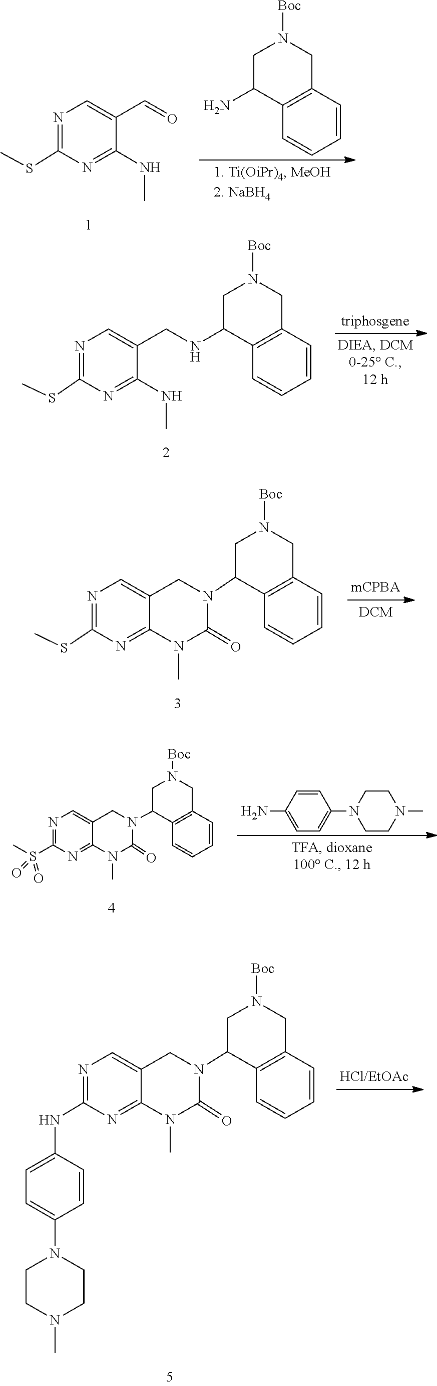

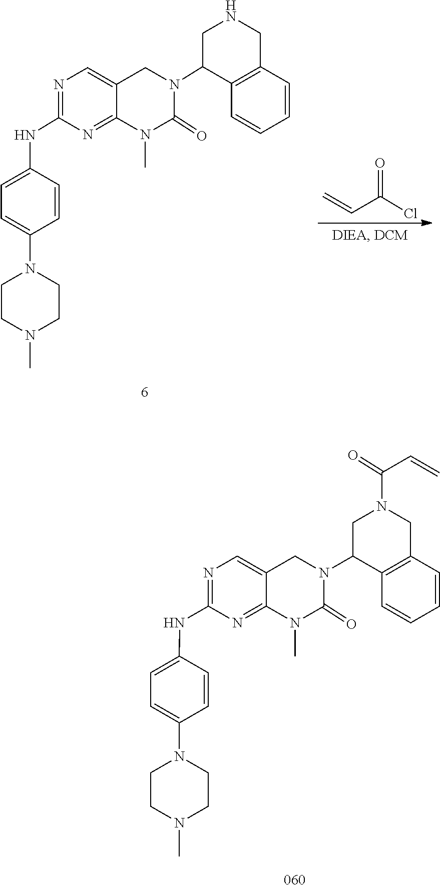

To a solution of isoquinolin-4-amine (3 g, 20.81 mmol, 1 eq) in Py (150 mL) was added TFAA (4.37 g, 20.81 mmol, 2.89 mL, 1 eq) at 0° C., and the reaction mixture was stirred at 100° C. for 1 hr under N₂. LCMS showed the reaction was completed and desired product was detected. The reaction mixture was quenched by saturated NaHCO3 solution (60 mL) and extracted with ethyl acetate (50 mL×3). The combined organic phase was washed with brine (50 mL×1), dried over anhydrous Na2SO4, filtered and concentrated give a residue. The residue was purified by flash silica gel chromatography (ISCO®; 12 g SepaFlash® Silica Flash Column, Eluent of 0˜40% Ethyl acetate/Petroleum ether gradient @ 120 mL/min) to afford 2,2,2-trifluoro-N-(4-isoquinolyl)acetamide (4 g, 16.65 mmol, 80.04% yield, 100% purity) was obtained as a white solid.

To a solution of 2,2,2-trifluoro-N-(4-isoquinolyl)acetamide (4 g, 16.65 mmol, 1 eq) in AcOH (80 mL) was added PtO₂ (0.8 g, 3.52 mmol, 2.12e-1 eq) was added under nitrogen atmosphere. The reaction mixture placed under H2 (15 Psi) at 20° C. for 10 h. LCMS showed the reaction was completed and desired product was detected. The catalyst was removed by filtration and the filtrate was concentrated in vacuo. The reaction mixture was quenched by saturated NaHCO₃solution (80 mL) and extracted with ethyl acetate (80 mL×3). The combined organic phase was washed with brine (80 mL×1), dried over anhydrous Na₂SO₄, filtered and concentrated give 2,2,2-trifluoro-N-(1,2,3,4-tetrahydroisoquinolin-4-yl)acetamide (3.6 g, crude) was obtained as a white solid, which was used in next step directly.

To a solution of 2,2,2-trifluoro-N-(1,2,3,4-tetrahydroisoquinolin-4-yl)acetamide (3.6 g, 14.74 mmol, 1 eq) in THE (40 mL) were added Boc₂O (3.54 g, 16.22 mmol, 3.73 mL, 1.1 eq) and TEA (1.79 g, 17.69 mmol, 2.46 mL, 1.2 eq), and the reaction mixture was stirred at 20° C. for 12 hr under N₂. LC-MS showed the desired compound was detected. The reaction mixture was concentrated to give a residue. The residue was diluted with H₂O (50 mL) and extracted with EtOAc (50 mL×3). The combined organic layers were dried over Na₂SO₄, filtered and the filtrate was concentrated to give a residue. The residue was purified by flash silica gel chromatography (ISCO®; 20 g SepaFlash® Silica Flash Column, Eluent of 0-8% Ethyl acetate/Petroleum ether gradient @ 120 mL/min) to give tert-butyl 4-[(2,2,2-trifluoroacetyl)amino]-3,4-dihydro-1H-isoquinoline-2-carboxylate (4.1 g, 11.91 mmol, 80.77% yield, 100.00% purity) was obtained as a white solid.

To a solution of tert-butyl 4-[(2,2,2-trifluoroacetyl)amino]-3,4-dihydro-1 H-isoquinoline-2-carboxylate (2.01 g, 5.84 mmol, 1 eq) in MeOH (16 mL) and H₂O (4 mL) was added K2CO3 (1.61 g, 11.67 mmol, 2 eq) at 20° C. The mixture was stirred at 50° C. for 12 hr. LCMS showed the reaction was completed and desired product. The reaction mixture was concentrated to give a residue. The residue was diluted with H₂O (20 mL) and extracted with EtOAc (20 mL×3). The combined organic layers were dried over Na₂SO₄, filtered and the filtrate was concentrated to give tert-butyl 4-amino-3,4-dihydro-1 H-isoquinoline-2-carboxylate (1.3 g, crude) was obtained as a yellow oil, which was used in next step directly.

To a solution of tert-butyl 4-amino-3,4-dihydro-1 H-isoquinoline-2-carboxylate (1 g, 4.03 mmol, 1 eq) in MeOH (25 mL) was added 4-(methylamino)-2-methylsulfanyl-pyrimidine-5-carbaldehyde (811.67 mg, 4.43 mmol, 1.1 eq) and tetraisopropoxytitanium (3.43 g, 12.08 mmol, 3.57 mL, 3 eq). The mixture was stirred at 25° C. for 12 hr. Then NaBH₄ (914.12 mg, 24.16 mmol, 6 eq) was added to the mixture at 0° C. The mixture was stirred at 25° C. for 3 hr. LCMS showed the reaction was completed and desired product. The reaction mixture was concentrated under reduced pressure to give a residue. The reaction mixture was quenched by saturated NH₄Cl solution (15 mL) and extracted with ethyl acetate (15 mL×3). The combined organic phase was washed with brine (15 mL×1), dried over anhydrous Na₂SO₄, filtered and concentrated give a residue. The residue was purified by flash silica gel chromatography (ISCO®; 4 g SepaFlash® Silica Flash Column, Eluent of 0-18% Ethyl acetate/Petroleum ether gradient @ 120 mL/min) to give tert-butyl 4-[[4-(methylamino)-2-methylsulfanyl-pyrimidin-5-yl]methylamino]-3,4-dihydro-1 H-isoquinoline-2-carboxylate (1 g, 2.33 mmol, 57.93% yield, 96.95% purity) was obtained as a yellow solid. 1H NMR (400 MHz, DMSO-d6) δ ppm 1.44 (s, 9H) 2.42 (s, 3H) 2.89 (d, J=4.75 Hz, 3H) 3.31-3.32 (m, 2H) 3.57-3.76 (m, 3H) 3.91 (br s, 1H) 4.28-4.44 (m, 1H) 4.67 (br d, J=16.88 Hz, 1H) 7.07-7.43 (m, 5H) 7.80 (s, 1H).

To a solution of bis(trichloromethyl) carbonate (928.34 mg, 3.13 mmol, 1eq) in DCM (13 mL) was added DIEA (2.02 g, 15.64 mmol, 2.72 mL, 5eq) and tert-butyl 4-[[4-(methylamino)-2-methylsulfanyl-pyrimidin-5-yl] methylamino]-3,4-dihydro-1 Hisoquinoline-2-carboxylate (1.3 g, 3.13 mmol, 1eq) at 0° C.. The mixture was stirred at 25° C. for 12 hr. The mixture was stirred under H₂ (15 psi) at 25° C. for 1 hours. LCMS showed the reaction was completed and desired product. The reaction mixture was quenched by saturated NaHCO₃solution (30 mL) and extracted with DCM (30 mL×3). The combined organic phase was washed with brine (30 mL×1), dried over anhydrous Na2SO₄, filtered and concentrated give a residue. The residue was purified by flash silica gel chromatography (ISCO®; 12 g SepaFlash® Silica Flash Column, Eluent of 0-18% Ethyl acetate/Petroleum ether gradient @ 120 mL/min) to give tert-butyl 4-(1-methyl-7-methylsulfanyl-2-oxo-4H-pyrimido[4,5-d]pyrimidin-S-yl)-3,4-dihydro-1H-isoquinoline-2-carboxylate (1.1 g, 2.34 mmol, 74.83% yield, 93.97% purity) was obtained as a white solid. 1H NMR (400 MHz, DMSO-d6) δ ppm 1.17 (t, J=7.13 Hz, 1H) 1.35 (br s, 9H) 1.99 (s, 1H) 2.48 (s, 3H) 3.28-3.31 (m, 1H)3.67 (br d, J=3.00 Hz, 1H) 3.85 (br d, J=14.76 Hz, 1H) 4.03 (q, J=7.09 Hz, 1H) 4.46-4.57 (m, 1H) 4.64-4.73 (m, 1H) 5.46 (br s, 1H) 7.12-7.38 (m, 4H) 8.07 (s, 1H).

To a solution of tert-butyl 4-(1-methyl-7-methylsulfanyl-2-oxo-4H-pyrimido[4,5-d]pyrimidin-S-yl)-3,4-dihydro-1H-isoquinoline-2-carboxylate (1.1 g, 2.49 mmol, 1 eq) in DCM (15 mL) was added MCPBA (1.26 g, 6.23 mmol, 85% purity, 2.5 eq) at 0° C. The mixture was stirred at 25° C. for 3 hr. LCMS showed the reaction was completed and desired product. The reaction mixture was quenched by saturated Na2SO₃ solution (30 mL) and extracted with DCM (20 mL×3). The combined organic phase was washed with NaHCO₃ (20 mL×1), dried over anhydrous Na2SO₄, filtered and concentrated give a residue. The residue was purified by flash silica gel chromatography (ISCO®; 4 g SepaFlash® Silica Flash Column, Eluent of 0-50% Ethyl acetate/Petroleum ether gradient @ 120 mL/min) to give tert-butyl 4-(1-methyl-7-methylsulfonyl-2-oxo-4H-pyrimido[4,5-d]pyrimidin-S-yl)-3,4-dihydro-1 H-isoquinoline-2-carboxylate (0.7 g, 1.48 mmol, 59.34% yield, 100.00% purity) was obtained as a white solid. 1H NMR (400 MHz, DMSO-d6) δ ppm 0.84 (br d, J=6.88 Hz, 1H) 1.35 (br s, 9H) 1.99 (s, 1H) 3.36 (s, 6H) 3.78-3.78 (m, 1H) 4.10-4.30 (m, 1H) 4.50-4.71 (m, 2H) 5.48 (br s, 1H) 7.21-7.39 (m, 4H) 8.44 (s, 1H).

To a solution of tert-butyl 4-(1-methyl-7-methylsulfonyl-2-oxo-4H-pyrimido[4,5-d]pyrimidin-S-yl)-3,4-dihydro-1 H-isoquinoline-2-carboxylate (200 mg, 422.35 umol, 1 eq) and 4-(4-methylpiperazin-1-yl)aniline (161.57 mg, 844.69 umol, 2 eq) in dioxane (1 mL) was added TFA (72.24 mg, 633.52 umol, 46.91 uL, 1.5 eq), and the reaction mixture was stirred at 100° C. for 12 hr under N₂. LCMS showed some reactant was remained and desired product was detected. The reaction mixture was concentrated to give a residue. The residue was dissolved in DMF(3 mL), and the resulting solution was purified by prep-HPLC (neutral condition, column: Waters Xbridge Prep OBD C18 150*40 mm*10 um;mobile phase: [water(NH₄HCO₃)-ACN];B %: 35%-65%, 8 min) to give tert-butyl 4-[1-methyl-7-[4-(4-methylpiperazin-1-yl)anilino]-2-oxo-4H-pyrimido[4,5-d]pyrimidin-S-yl]-3,4-dihydro-1 H-isoquinoline-2-carboxylate (180 mg, 307.84 umol, 36.44% yield) was obtained as a white solid.

To a solution of tert-butyl 4-[1-methyl-7-[4-(4-methylpiperazin-1-yl)anilino]-2-oxo-4H-pyrimido[4,5-d] pyrimidin-S-yl]-3,4-dihydro-1H-isoquinoline-2-carboxylate (180 mg, 307.84 umol, 1 eq) in EtOAc (0.1 mL) was added HCl/EtOAc (4 M, 9.00 mL, 116.94 eq) at 20° C. And the reaction mixture was stirred at 20° C. for 1 hr. LCMS showed the reaction was completed and desired product. The reaction mixture was filtered and concentrated under reduced pressure to give 1-methyl-7-[4-(4-methylpiperazin-1-yl)anilino]-S-(1,2,3,4-tetrahydroisoquinolin-4-yl)-4H-pyrimido[4,5-d]pyrimidin-2-one (149 mg, crude, HCl) was obtained as a white solid, which was used in next step directly.

To a solution of 1-methyl-7-[4-(4-methylpiperazin-1-yl)anilino]-S-(1,2,3,4-tetrahydroisoquinolin-4-yl)-4H-pyrimido[4,5-d]pyrimidin-2-one (140 mg, 268.69 umol, 1 eq, HCl) and DIEA (104.18 mg, 806.06 umol, 140.40 uL, 3eq) in DCM (2 mL) was dropwise added a DCM diluted solution of prop-2-enoyl chloride (36.48 mg, 403.03 umol, 32.86 uL, 1.5 eq) at 0° C. The mixture was stirred at 0° C. for 1 hr. LCMS showed the reaction completed and desired product was detected. The mixture was concentrated to get crude residue. The residue was dissolved in DMSO(1.5 mL), and the resulting solution was purified by prep-HPLC (FA condition, column:C18-1 150*30 mm*5 um;mobile phase: [water(FA)-ACN];B %:10%-40%, 10 min) to afford 3-(2-acryloyl-1,2,3,4-tetrahydroisoquinolin-4-yl)-1-methyl-7-((4-(4-methylpiperazin-1-yl)phenyl)amino)-3,4-dihydropyrimido[4,5-d]pyrimidin-2(1H)-one (22 mg, 44.13 umol, 49.04% yield, 100% purity) as a white solid.

### Procedure for Preparation of Compound 085

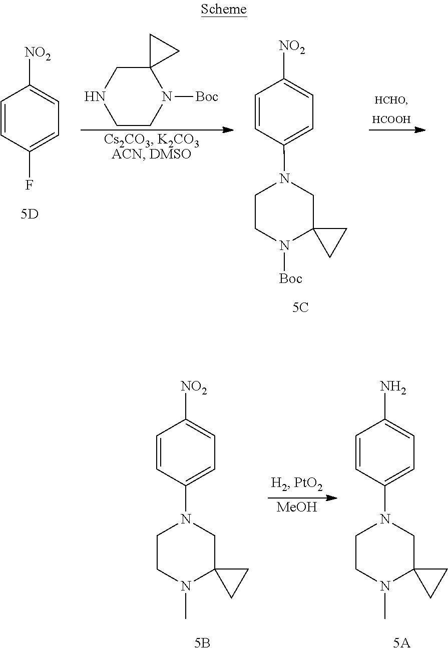

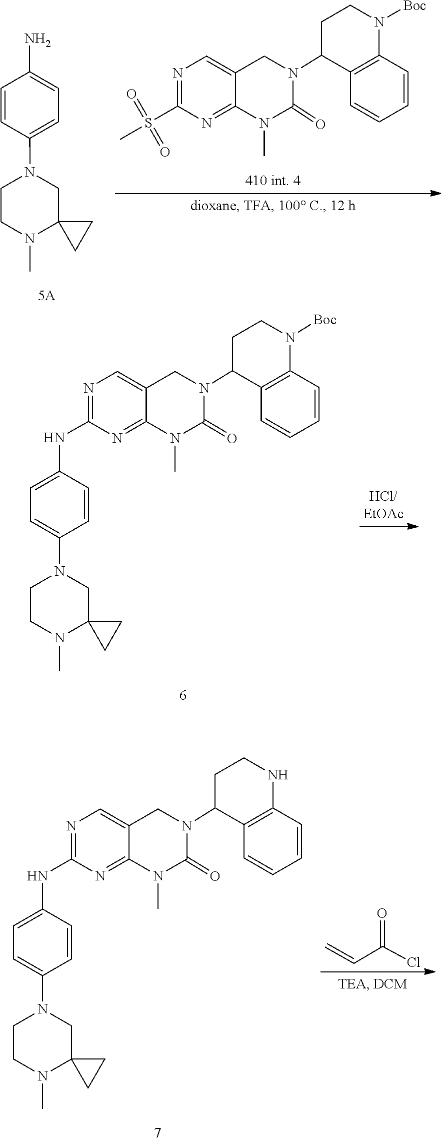

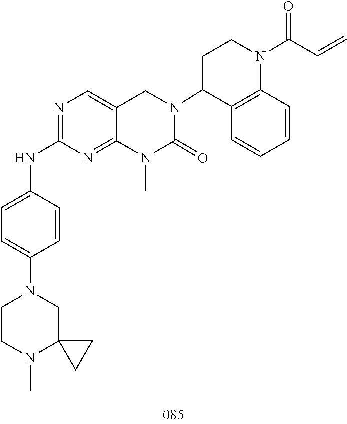 [SMILES: C=CC(=O)N1CCC(N2Cc3cnc(Nc4ccc(N5CCN(C)C6(CC6)C5)cc4)nc3N(C)C2=O)c2ccccc21]

To a solution of tert-butyl 4, 7-diazaspiro [2.5] octane-4-carboxylate (1 g, 4.7 mmol, 1 eq) in MeCN (20 mL) was added K₂CO₃ (2 g, 14.1 mmol, 3 eq) and 1-fluoro-4-nitro-benzene (1 g, 7.1 mmol, 749.6 uL, 1.5 eq). The mixture was stirred at 100° C. for 12 hr. Cs₂CO₃ (2.3 g, 7.1 mmol, 1.5 eq) and DMSO (10 mL) was added to the mixture, the mixture was stirred at 100° C. for another 12 hr. LC-MS showed the desired compound was detected. The reaction mixture was partitioned between NaHCO₃ 100 mL and ethyl acetate (100 mL×3). The organic phase was separated, washed with brine (50 mL×3), dried over Na₂SO₄, filtered and concentrated under reduced pressure to give a residue. The residue was purified by flash silica gel chromatography (20 g Silica Flash Column, Eluent of 0-50% Ethyl acetate/Petroleum ether gradient @ 120 mL/min) to give tert-butyl 7-(4-nitrophenyl)-4, 7-diazaspiro [2.5] octane-4-carboxylate (1 g, 2.8 mmol, 58.6% yield, 92.1% purity) as a yellow solid.

A mixture of tert-butyl 7-(4-nitrophenyl)-4,7-diazaspiro[2.5]octane-4-carboxylate (950 mg, 2.9 mmol, 1 eq) in HCOOH (12 mL) and HCHO (3 mL, 37% solution) was stirred at 100° C. for 1 hr. LC-MS showed the desired compound was detected. The reaction mixture was concentrated under reduced pressure to give a residue. The residue was purified by flash silica gel chromatography (20 g Silica Flash Column, Eluent of 0-10% Dichloromethane/Methanol @ 100 mL/min) to give 4-methyl-7-(4-nitrophenyl)-4, 7-diazaspiro [2.5] octane (440 mg, 1.8 mmol, 62.4% yield, 100% purity) as a yellow solid.

To a solution of 4-methyl-7-(4-nitrophenyl)-4, 7-diazaspiro [2.5]octane (380 mg, 1.5 mmol, 1 eq) in MeOH (10 mL) was added PtO₂ (69.8 mg, 307.3 umol, 0.2 eq) under N₂. The suspension was degassed under vacuum and purged with H₂ several times. The mixture was stirred under H₂ (15 psi) at 25° C. for 2 hr. LC-MS showed the desired compound was detected. The reaction mixture was concentrated under reduced pressure to give a residue. The residue was purified by prep-HPLC (NH₄HCO₃ condition column: Waters Xbridge BEH C18 100*30 mm*10 um; mobile phase: [water (NH₄HCO₃)-ACN]; B %: 1%-30%, 8 min) to give 4-(4-methyl-4, 7-diazaspiro [2.5] octan-7-yl) aniline (340 mg, 1.5 mmol, 99.7% yield, 97.9% purity) as a brown solid.

To a solution of tert-butyl 4-(1-methyl-7-methylsulfonyl-2-oxo-4H-pyrimido[4,5-d]pyrimidin-S-yl)-3,4-dihydro-2H-quinoline-1-carboxylate (440 mg, 929.2 umol, 1 eq) in dioxane (4 mL) was added TFA (158.9 mg, 1.4 mmol, 103.19 uL, 1.5 eq) and 4-(4-methyl-4,7-diazaspiro[2.5]octan-7-yl)aniline (242.3 mg, 1.1 mmol, 1.2 eq). The mixture was stirred at 100° C. for 12 hr. LC-MS showed the desired compound was detected. The reaction mixture was concentrated under reduced pressure to give a residue. The residue was purified by prep-HPLC (FA condition column: Phenomenex Luna C18 75*30 mm*3 um; mobile phase: [water(FA)-ACN]; B %: 10%-40%, 8 min) to give tert-butyl 4-[1-methyl-7-[4-(4-methyl-4,7-diazaspiro[2.5]octan-7-yl)anilino]-2-oxo-4H-pyrimido[4,5-d]pyrimidin-S-yl]-3,4-dihydro-2H-quinoline-1-carboxylate (300 mg, 491.2 umol, 52.9% yield) as a brown solid.

A mixture of tert-butyl 4-[1-methyl-7-[4-(4-methyl-4,7-diazaspiro[2.5]octan-7-yl)anilino]-2-oxo-4H-pyrimido[4,5-d]pyrimidin-S-yl]-3,4-dihydro-2H-quinoline-1-carboxylate (250 mg, 409.3 umol, 1 eq) in HCl/EtOAc (3 mL, 4 M) was stirred at 25° C. for 1 hr. LC-MS showed the desired compound was detected. The reaction mixture was concentrated under reduced pressure to give 1-methyl-7-[4-(4-methyl-4,7-diazaspiro[2.5]octan-7-yl)anilino]-S-(1,2,3,4-tetrahydroquinolin-4-yl)-4H-pyrimido[4,5-d]pyrimidin-2-one (250 mg, crude, HCl) as a white solid.

To a solution of 1-methyl-7-[4-(4-methyl-4,7-diazaspiro[2.5]octan-7-yl)anilino]-S-(1,2,3,4-tetrahydroquinolin-4-yl)-4H-pyrimido[4,5-d]pyrimidin-2-one (250 mg, 456.9 umol, 1 eq, HCl) in DCM (5 mL) was added TEA (138.7 mg, 1.4 mmol, 190.8 uL, 3 eq) and prop-2-enoyl chloride (82.7 mg, 913.9 umol, 74.5 uL, 2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 200*40 mm*10 um;mobile phase: [water(FA)-ACN]; B %: 5%-40%, 8 min) and (NH₄HCO₃ condition, column: Waters Xbridge Prep OBD C18 150*40 mm*10 um; mobile phase: [water (NH₄HCO₃)-ACN]; B %: 25%-55%, 8 min) to give 1-methyl-7-[4-(4-methyl-4,7-diazaspiro[2.5]octan-7-yl)anilino]-S-(1-prop-2-enoyl-3,4-dihydro-2H-quinolin-4-yl)-4H-pyrimido[4,5-d]pyrimidin-2-one (55.1 mg, 97.6 umol, 21.4% yield, 100% purity) as a white solid.

### Procedure for Preparation of Compound 086

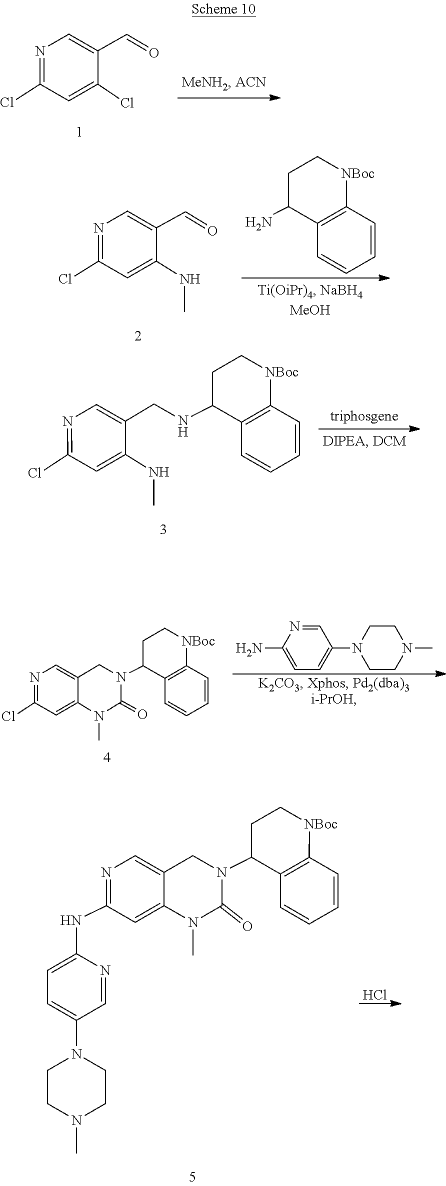

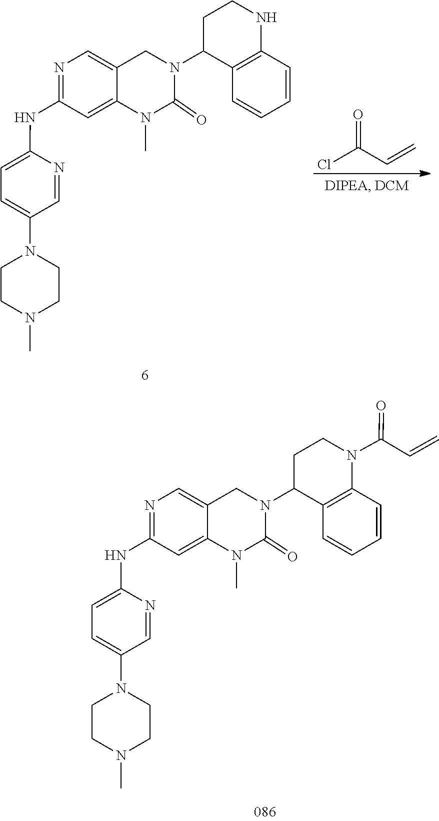

To a solution of 4,6-dichloropyridine-S-carbaldehyde (5 g, 28.41 mmol, 1 eq) in ACN (50 mL) was added MeNH2 (12.58 g, 162.03 mmol, 40% THE solution, 5.70 eq) at 20° C. The mixture was stirred at 50° C. for 12 hr. LCMS showed the reaction was completed and desired product was detected. The mixture was concentrated under vacuum. The residue was diluted with 20 mL H₂O and extracted with EtOAc (15 mL*3). The combined organic phase was dried with anhydrous Na₂SO₄, the mixture was filtered and the filtrate was concentrated under vacuum. The residue was purified by flash silica gel chromatography (ISCO®; 40 g SepaFlash® Silica Flash Column, Eluent of 0-50% Ethyl acetate/Petroleum ether gradient @ 200 mL/min)(Petroleum ether/Ethyl acetate=3/1, P1 Rf=0.33) to give 2.5 g desired compound. The residue was further purified by prep-HPLC (neutral condition)column: Phenomenex luna C18 (250*70 mm, 15 um);mobile phase: [water(FA)-ACN];B %: 2%-50%, 20 min to give 6-chloro-4-(methylamino)pyridine-S-carbaldehyde (2.2 g, 12.90 mmol, 45.39% yield) as a white solid.

To a solution of 6-chloro-4-(methylamino)pyridine-S-carbaldehyde (400 mg, 2.34 mmol, 1 eq) in MeOH (10 mL) was added Ti(i-PrO)₄ (2.00 g, 7.03 mmol, 2.08 mL, 3 eq) and tert-butyl 4-amino-3,4-dihydro-2H-quinoline-1-carboxylate (582.24 mg, 2.34 mmol, 1 eq) at 20° C.. The mixture was stirred at 30° C. for 12 hr. Then NaBH₄ (532.24 mg, 14.07 mmol, 6 eq) was added to the mixture at 0° C. The mixture was stirred at 20° C. for 3 hr. LCMS showed the reaction was completed and desired product was detected. The reaction mixture was quenched with H₂O (20 mL) at 0° C., The reaction mixture was diluted with 30 mL MeOH. The mixture was filtered and the filtrate was concentrated under vacuum. The residue was diluted with 20 mL H₂O and extracted with EtOAc(20 mL*3). The combined organic phase was dried with anhydrous Na2SO₄, the mixture was filtered and the filtrate was concentrated under vacuum. The residue was purified by flash silica gel chromatography (ISCO®; 20 g SepaFlash® Silica Flash Column, Eluent of 20-80% Ethyl acetate/Petroleum ether gradient @ 40 mL/min) to give tert-butyl 4-[[6-chloro-4-(methylamino)-S-pyridyl]methylamino]-3,4-dihydro-2H-quinoline-1-carboxylate (900 mg, 2.23 mmol, 95.27% yield) as a white solid.

To a solution of tert-butyl 4-[[6-chloro-4-(methylamino)-S-pyridyl]methylamino]-3,4-dihydro-2H-quinoline-1-carboxylate (900 mg, 2.23 mmol, 1 eq) in DCM (50 mL) was added DIEA (866.06 mg, 6.70 mmol, 1.17 mL, 3 eq) and bis(trichloromethyl) carbonate (662.85 mg, 2.23 mmol, 1 eq) at 0° C. The mixture was stirred at 25° C. for 12 hr. LC-MS showed the desired compound mass peak was detected. The reaction solution is quenched by adding 10 mL NaHCO₃ aqueous solution at 0° C., and then adding DCM (8 ml*3) for extraction. The combined organic phase was dried with anhydrous Na₂SO₄, the mixture was filtered and the filtrate was concentrated under vacuum. The residue was purified by flash silica gel chromatography (ISCO®; 20 g SepaFlash® Silica Flash Column, Eluent of 0-40% Ethyl acetate/Petroleum ether gradient @ 40 mL/min) to give tert-butyl 4-(7-chloro-1-methyl-2-oxo-4H-pyrido[4,3-d]pyrimidin-S-yl)-3,4-dihydro-2H-quinoline-1-carboxylate (630 mg, 1.47 mmol, 65.76% yield) as a yellow oil. 1 H NMR (400 MHz, CHLOROFORM-d) δ ppm 7.92 (s, 1H), 7.82 (br d, J=8.31 Hz, 1H), 7.28-7.33 (m, 1H), 7.12-7.17 (m, 1H), 7.04-7.10 (m, 1H), 6.84 (s, 1H), 5.88 (br t, J=8.31 Hz, 1H), 4.30 (dt, J=13.20, 4.03 Hz, 1H), 4.18 (d, J=6.60 Hz, 1H), 4.01-4.09 (m, 1H), 3.52-3.62 (m, 1H), 3.41 (s, 3H), 2.22-2.32 (m, 1H), 2.10 (s, 2H), 1.61 (s, 9H)

To a solution of tert-butyl 4-(7-chloro-1-methyl-2-oxo-4H-pyrido[4,3-d]pyrimidin-S-yl)-3,4-dihydro-2H-quinoline-1-carboxylate (600 mg, 1.40 mmol, 1 eq) and 5-(4-methylpiperazin-1-yl)pyridin-2-amine (537.90 mg, 2.80 mmol, 2 eq) in i-PrOH (20 mL) was added Pd2(dba)₃ (64.05 mg, 69.94 umol, 0.05 eq), K₂CO₃ (638.01 mg, 4.62 mmol, 3.3 eq) and XPhos (46.68 mg, 97.92 umol, 0.07 eq) at 20° C. under N₂ atmosphere. The mixture evacuated and recharged with nitrogen for 3 times, and was then stirred at 110° C. for 4 hr under N₂ atmosphere. LCMS showed the reaction was completed and desired product peak formation. The mixture was concentrated under vacuum. The residue was diluted with 10 mL H₂O and extracted with EtOAc(6 mL*3). The combined organic phase was dried with anhydrous Na₂SO₄, the mixture was filtered and the filtrate was concentrated under vacuum.

The residue was purified by prep-HPLC(basic condition) (column: Waters Xbridge Prep OBD C18 150*40 mm*10 um;mobile phase: [water(NH₄HCO₃)-ACN];B %: 30%-60%, 8 min) to give tert-butyl 4-[1-methyl-7-[[5-(4-methylpiperazin-1-yl)-2-pyridyl]amino]-2-oxo-4H-pyrido[4,3-d]pyrimidin-S-yl]-3,4-dihydro-2H-quinoline-1-carboxylate (320 mg, 547.28 umol, 39.12% yield) as a white solid.

1H NMR (400 MHz, CHLOROFORM-d) δ ppm 7.98 (s, 1H), 7.76 (d, J=8.38 Hz, 1H), 7.71 (s, 1H), 7.28 (d, J=1.50 Hz, 2H), 7.20-7.25 (m, 1H), 7.18 (s, 1H), 7.14 (d, J=7.63 Hz, 1H), 6.98-7.04 (m, 1H), 5.85 (dd, J=9.57, 7.32 Hz, 1H), 4.24 (dt,J=13.29, 4.24 Hz, 1H), 4.03-4.09 (m, 1H), 3.90-3.96 (m, 1H), 3.49-3.58 (m, 1H), 3.39 (s, 3H), 3.13-3.20 (m, 4H), 2.56-2.64 (m, 4H), 2.37 (s, 3H), 2.18-2.27 (m, 1H), 1.98-2.10 (m, 1H), 1.56 (s, 9H)

To a solution of tert-butyl 4-[1-methyl-7-[[5-(4-methylpiperazin-1-yl)-2-pyridyl]amino]-2-oxo-4H-pyrido[4,3-d]pyrimidin-S-yl]-3,4-dihydro-2H-quinoline-1-carboxylate (300 mg, 513.07 umol, 1 eq) in EtOAc (5 mL) was added HCl/EtOAc (4 M, 7.70 mL, 60 eq) at 20° C. . The mixture was stirred at 20° C. for 1 hr. LCMS showed the reaction was completed and desired product mass peak formation. The reaction mixture was concentrated under reduced pressure to remove solvent to give 1-methyl-7-[[5-(4-methylpiperazin-1-yl)-2-pyridyl]amino]-3-(1,2,3,4-tetrahydroquinolin-4-yl)-4H-pyrido[4,3-d]pyrimidin-2-one (460 mg, crude, HCl) as a white solid.

To a solution of 1-methyl-7-[[5-(4-methylpiperazin-1-yl)-2-pyridyl]amino]-S-(1,2,3,4-tetrahydroquinolin-4-yl)-4H-pyrido[4,3-d]pyrimidin-2-one (460 mg, 949.25 umol, 1 eq) in DCM (10 ml) was added DIPEA (368.04 mg, 2.85 mmol, 496.01 uL, 3 eq) and prop-2-enoyl chloride (42.96 mg, 474.62 umol, 38.70 uL, 0.5 eq) in DCM (0.14 mL) under N₂ atmosphere at 0° C. The mixture was stirred at 0° C. for 1 hr under N₂ atmosphere. LCMS showed the reaction completed and desired product mass peak was detected. The reaction mixture was concentrated under reduced pressure. The residue was purified by prep-HPLC (FA condition)(column: Phenomenex C18 75*30 mm*3 um;mobile phase: [water(FA)-ACN];B %: 15%-45%, 8 min) to give 1-methyl-7-[[5-(4-methylpiperazin-1-yl)-2-pyridyl]amino]-S-(1-prop-2-enoyl-3,4-dihydro-2H-quinolin-4-yl)-4Hpyrido[4,3-d]pyrimidin-2-one (49.6 mg, 85.64 umol, 9.02% yield, 93% purity) as a white solid.

### Procedure for Preparation of Compound 089

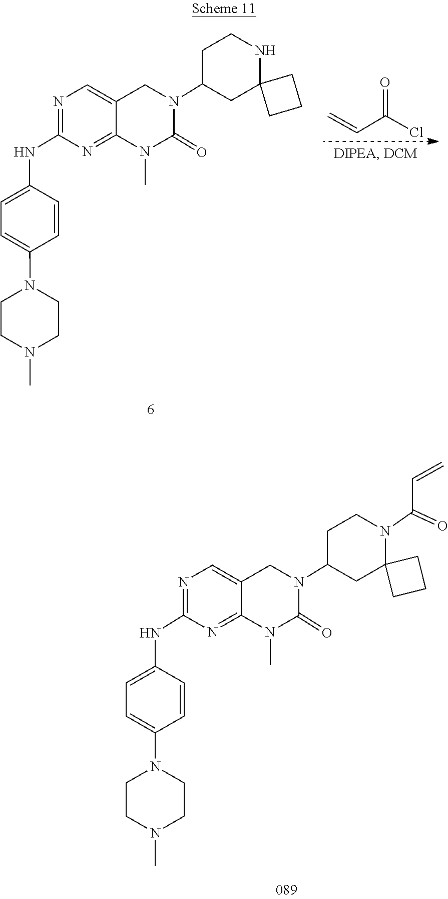

To a solution of 3-(5-azaspiro[3.5]nonan-8-yl)-1-methyl-7-[4-(4-methylpiperazin-1-yl)anilino]-4H-pyrimido[4,5-d]pyrimidin-2-one (110 mg, 214.39 umol, 1 eq, HCl) in DCM(3 ML) was added DIPEA (83.13 mg, 643.18 umol, 112.03 uL, 3 eq). The prop-2-enoyl chloride (29.11 mg, 321.59 umol, 26.22 uL, 1.5 eq) in DCM (1 ml) was added to the mixture under N₂ atmosphere at 0° C. The mixture was stirred 0° C. for 1 hr under N₂ atmosphere. LCMS showed the reaction completed and desired product was detected. The reaction mixture was concentrated under reduced pressure to remove solvent. The residue was purified by prep-HPLC (FA condition, column: Phenomenex Luna C18 75*30 mm*3 um;mobile phase:[water(FA)-ACN];B %: 1%-30%, 8 min) to give 1-methyl-7-[4-(4-methylpiperazin-1-yl)anilino]-S-(5-prop-2-enoyl-5-azaspiro[3.5]nonan-8-yl)-4H-pyrimido[4,5-d]pyrimidin-2-one (41.8 mg, 67.35 umol, 31.41% yield, 92.92% purity, FA) as a yellow solid.

### Procedure for Preparation of Compound 092

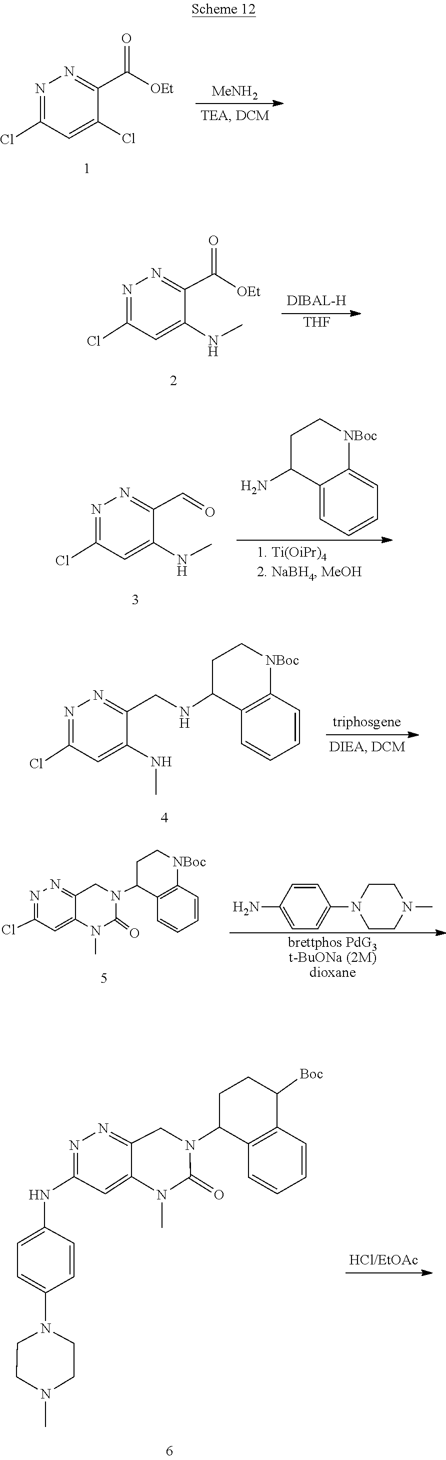

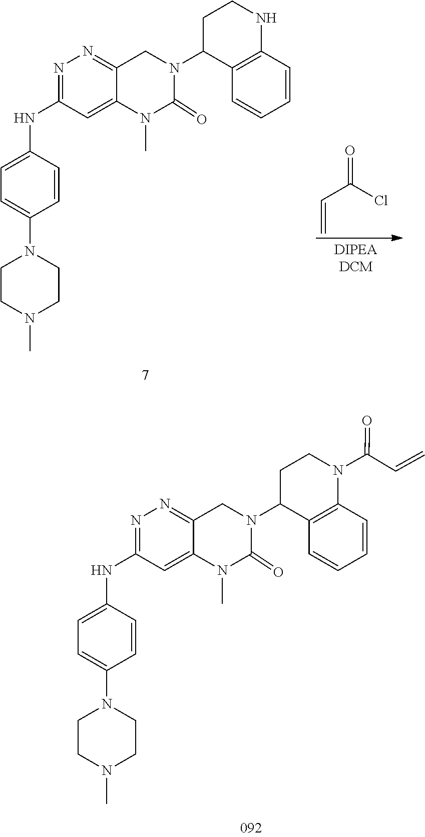

To a solution of methyl 4,6-dichloropyridazine-S-carboxylate (5 g, 24.2 mmol, 1 eq) in DCM (50 mL) was added TEA (6.4 g, 62.8 mmol, 8.7 mL, 2.6 eq), methanamine hydrochloride (2.1 g, 31.4 mmol, 1.3 eq). The mixture was stirred at 25° C. for 12 h. LC-MS showed desired compound was detected. The mixture was filtered and the filtrate was concentrated to get a residue. The residue was purified by column chromatography (SiO₂, Petroleum ether/Ethyl acetate=10/1 to 1/1) to give compound methyl 6-chloro-4-(methylamino) pyridazine-S-carboxylate (4.5 g, 22.3 mmol, 92.4% yield) as a pale yellow solid.

To a mixture of methyl 6-chloro-4-(methylamino)pyridazine-S-carboxylate (4 g, 19.8 mmol, 1 eq) in THE (200 mL) was added DIBAL-H (1 M, 39.7 mL, 2 eq) at −78° C., the mixture was stirred at −78° C. for 2 h. LC-MS showed desired compound was detected. The mixture was quenched by Sat. NH₄Cl at 0° C., then the mixture was filtered and the filtrate was extracted by ethyl acetate (30 mL×3), the combined organic layer was washed by brine (20 mL×2) and dried by Na₂SO₄, filtered and the filtrate was concentrated to get a residue. The crude product was triturated with (petroleum ether/ethyl acetate=3/1), 40 mL at 25° C. for 120 min to give compound 6-chloro-4-(methylamino)pyridazine-S-carbaldehyde (1.2 g, 7.0 mmol, 35.2% yield) as a brown solid.

To a solution of 6-chloro-4-(methylamino)pyridazine-S-carbaldehyde (950 mg, 5.5 mmol, 1 eq) and tert-butyl 4-amino-3,4-dihydro-2H-quinoline-1-carboxylate (1.4 g, 5.5 mmol, 1 eq) in MeOH (50 mL) was added Ti(i-PrO)₄ (4.7 g, 16.6 mmol, 4.9 mL, 3 eq), the mixture was stirred at 25° C. for 12 h, and then NaBH₄ (1.26 g, 33.2 mmol, 6 eq). The mixture was stirred at 25° C. for 12 h. LC-MS showed desired compound was detected. The mixture was concentrated to get a residue and the residue was quenched by Sat. NH₄Cl 20 mL and extracted by ethyl acetate (20 mL×3), the organic layers was washed by brine (10 mL×2) and dried by Na₂SO₄, filtered and the filtrate was concentrated to get a residue. The residue was purified by column chromatography (SiO₂, Petroleum ether/Ethyl acetate=3/1 to 0/1) to give compound tert-butyl 4-[[6-chloro-4-(methylamino)pyridazin-S-yl]methylamino]-3,4-dihydro-2H-quinoline-1-carboxylate (1.1 g, 2.7 mmol, 49.1% yield) as an orange oil.

To a solution of tert-butyl 4-[[6-chloro-4-(methylamino)pyridazin-S-yl]methylamino]-3,4-dihydro-2H-quinoline-1-carboxylate (1.1 g, 2.7 mmol, 1 eq) and DIEA (1.06 g, 8.2 mmol, 1.4 mL, 3 eq) in DCM (50 mL) was added triphosgene (646.5 mg, 2.2 mmol, 0.8 eq) in DCM (5 mL) drop wised at 0° C. The mixture was stirred at 25° C. for 3 h. LC-MS showed desired compound was detected. The mixture was quenched by Sat. NaHCO₃ 10 mL, then the mixture was extracted by DCM (15 mL×3), the organic layers was washed by brine (10 mL×2) and dried by Na₂SO₄, the mixture was filtered, and the filtrate was concentrated to get a residue. The residue was purified by column chromatography (SiO₂, Petroleum ether/Ethyl acetate=3/1 to 1/1) to give tert-butyl 4-(3-chloro-5-methyl-6-oxo-8H-pyrimido[5,4-c]pyridazin-7-yl)-3,4-dihydro-2H-quinoline-1-carboxylate (600 mg, 1.4 mmol, 51.2% yield) as a yellow solid.

A mixture of tert-butyl 4-(3-chloro-5-methyl-6-oxo-8H-pyrimido[5,4-c]pyridazin-7-yl)-3,4-dihydro-2H-quinoline-1-carboxylate (200 mg, 465.2 umol, 1 eq), 4-(4-methylpiperazin-1-yl)aniline (177.0 mg, 930.5 umol, 2 eq), tBuONa (2 M, 697.8 uL, 3 eq), BrettPhos Pd G3 (42.2 mg, 46.5 umol, 0.1 eq) in dioxane (3 mL) was degassed and purged with N₂ for 3 times, and then the mixture was stirred at 90° C. for 12 h under N₂ atmosphere. LC-MS showed desired compound was detected. The reaction mixture was concentrated to get a residue. The residue was purified by prep-HPLC (FA condition; column: Phenomenex Luna C18 75*30 mm*3 um;mobile phase: [water(FA)-ACN];B %: 15%-55%, 8 min) to give tert-butyl 4-[5-methyl-S-[4-(4-methylpiperazin-1-yl)anilino]-6-oxo-8H-pyrimido[5,4-c]pyridazin-7-yl]-3,4-dihydro-2H-quinoline-1-carboxylate (130 mg, 222.3 umol, 47.8% yield) as a white solid.

To a solution of tert-butyl 4-[5-methyl-S-[4-(4-methylpiperazin-1-yl)anilino]-6-oxo-8H-pyrimido[5,4-c]pyridazin-7-yl]-3,4-dihydro-2H-quinoline-1-carboxylate (130 mg, 222.3 umol, 1 eq) in EtOAc (1 mL) was added HCl/EtOAc (1 mL, 4 M). The mixture was stirred at 25° C. for 1 h. LC-MS showed desired compound was detected. The mixture was concentrated to give 5-methyl-S-((4-(4-methylpiperazin-1-yl)phenyl)amino)-7-(1,2,3,4-tetrahydroquinolin-4-yl)-7,8-dihydropyrimido[5,4-c]pyridazin-6(5H)-one (130 mg, crude, HCl) as a yellow solid.

To a solution of 5-methyl-S-[4-(4-methylpiperazin-1-yl)anilino]-7-(1,2,3,4-tetrahydroquinolin-4-yl)-8H-pyrimido[5,4-c]pyridazin-6-one (100 mg, 191.9 umol, 1 eq, HCl) in DCM (1 mL) was added TEA (97.1 mg, 959.6 umol, 133.6 uL, 5 eq) and prop-2-enoyl chloride (17.4 mg, 191.9 umol, 15.7 uL, 1 eq). The mixture was stirred at 0° C. for 1 h. LC-MS showed desired compound was detected. The mixture was concentrated to get a residue and the residue was purified by prep-HPLC (FA condition;column: Phenomenex Luna C18 75*30 mm*3 um;mobile phase: [water(FA)-ACN];B %: 1%-40%, 8 min) to give 5-methyl-S-[4-(4-methylpiperazin-1-yl)anilino]-7-(1-prop-2-enoyl-3,4-dihydro-2H-quinolin-4-yl)-8H-pyrimido[5,4-c]pyridazin-6-one (16.3 mg, 23.9 umol, 12.5% yield, 86% purity, FA) as a white solid.

### Procedure for Preparation of Compound 094

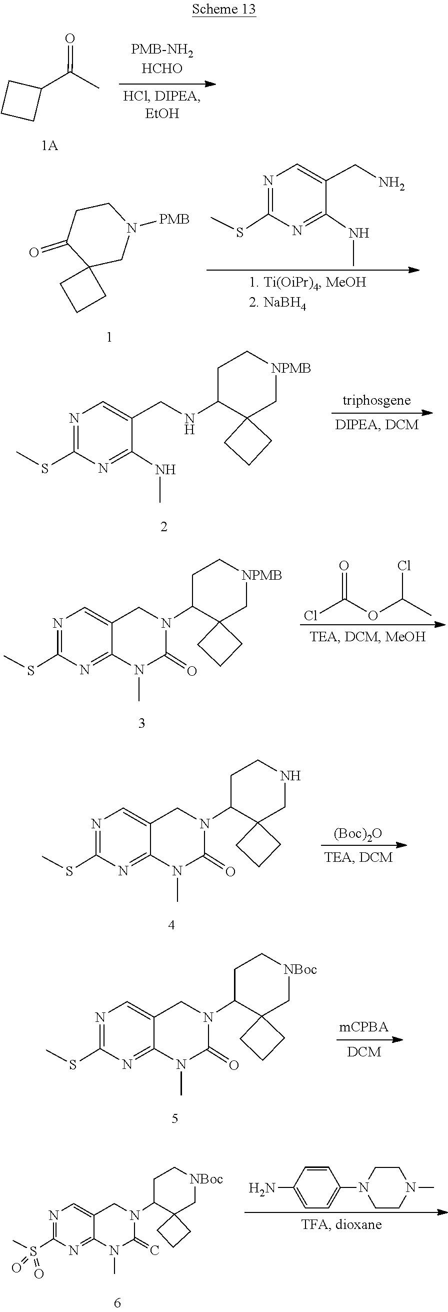

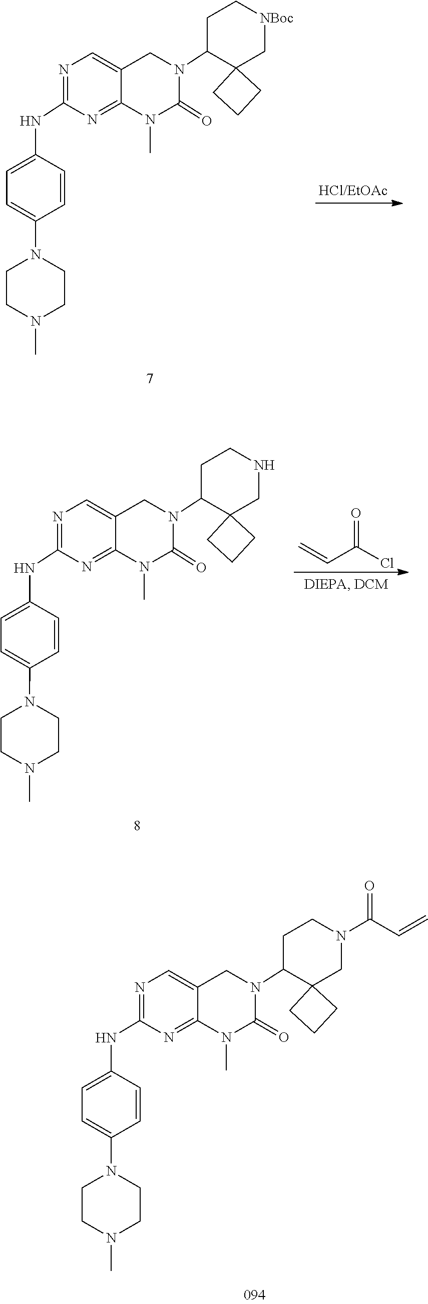

A solution of 1-cyclobutylethanone (1.02, 10.43 mmol, 1.14 mL, 1.06 eq) and HCl (12 M, 885.71 uL, 1.08 eq) in ethanol (5 mL) was heated at 80° C. to reflux. A mixture of (4-methoxyphenyl)methanamine (1.35,e9.84 mmol, 1.27 mL, 1 eq) and HCHO (1.80 g, 22.14 mmol, 1.65 mL, 37% solution, 2.25 eq) in ethanol (5 mL) was added to the refluxing reaction mixture. The reaction was stirred at 80° C. for 12 hrs. Then, HCHO (239.59 mg, 2.95 mmol, 219.80 uL, 37% solution, 0.3 eq) and DIPEA (1.36, 10.53 mmol, 1.83 mL, 1.07 eq) was added to the reaction mixture and heated at 80° C. for another 16 hrs. LCMS detected the formation of the desired mass in the major peak. The reaction mixture was concentrated under reduced pressure. The reaction concentrate was then diluted with 20 mL NaHCO₃ solution and extracted with EtOAc (20 mL*3). The combined organic phase was dried with anhydrous Na₂SO₄, filtered and concentrated under vacuum. The residue was purified by flash silica gel chromatography (ISCO®; 12 g SepaFlash® Silica Flash Column, Petroleum ether/Ethyl acetate=3/1, Rf=0.59 @ 30 mL/min) to give 6-[(4-methoxyphenyl)methyl]-6-azaspiro[3.5]nonan-9-one (1 g, 3.85 mmol, 39.2% yield) as a colorless oil was obtained as a yellow solid.

To a solution of 5-(aminomethyl)-N-methyl-2-methylsulfanyl-pyrimidin-4-amine (600 mg, 3.26 mmol, 1.41 eq) in MeOH (20 mL) was added Ti(i-PrO)₄ (1.97 g, 6.94 mmol, 2.05 mL, 3 eq) and 6-[(4-methoxyphenyl)methyl]-6-azaspiro[3.5]nonan-9-one (600.02 mg, 2.31 mmol, 1 eq) at 20° C.. The mixture was stirred at 20° C. for 12 hr. Then NaBH₄ (525.17 mg, 13.88 mmol, 6 eq) was added to the mixture at 0° C. The mixture was stirred at 20° C. for 3 hr. TLC indicated reactant 1 was consumed completely and new spots formed. LCMS detected the formation of the desired mass. The reaction mixture was quenched with NH₄Cl solution (20 mL) at 0° C., The reaction mixture was diluted with 40 mL MeOH. The mixture was filtered and the filtrate was concentrated under vacuum. The residue was diluted with 20 mL H₂O and extracted with EtOAc (20 mL*3). The combined organic phase was dried with anhydrous Na₂SO₄, the mixture was filtered and the filtrate was concentrated under vacuum. The residue was purified by flash silica gel chromatography (ISCO®; 20 g SepaFlash® Silica Flash Column, Eluent of 0-8% MeOH/DCM gradient @ 50 mL/min) to give 6-[(4-methoxyphenyl)methyl]-N-[[4-(methylamino)-2-methylsulfanyl-pyrimidin-5-yl]methyl]-6-azaspiro[3.5]nonan-9-amine (900 mg, 2.10 mmol, 90.97% yield) as a yellow solid.

To a solution of 6-[(4-methoxyphenyl)methyl]-N-[[4-(methylamino)-2-methylsulfanyl-pyrimidin-5-yl]methyl]-6-azaspiro[3.5]nonan-9-amine (720 mg, 1.68 mmol, 1 eq) in DCM(8 ml) was added DIPEA (652.86 mg, 5.05 mmol, 879.86 uL, 3 eq). The bis(trichloromethyl) carbonate (199.87 mg, 673.52 umol, 0.4 eq) in DCM(1 ml) was added to the mixture at 0° C. under the N₂. The mixture was stirred at 20° C. for 12 hr under the N₂. LCMS showed the reaction was completed and desired product was detected. The reaction mixture was quenched with saturated NAHCO3 aqueous solution(50 mL) at 0° C., then the mixture was extracted with EtOAc (30 mL*3). The combined organic phase was dried with anhydrous Na2SO₄, the mixture was filtered and the filtrate was concentrated under vacuum. The residue was purified by flash silica gel chromatography (ISCO®; 10 g SepaFlash® Silica Flash Column, Eluent of 0-8% MeOH/DCM@ 25 mL/min) to give 3-[6-[(4-methoxyphenyl)methyl]-6-azaspiro[3.5]nonan-9-yl]-1-methyl-7-methylsulfanyl-4H-pyrimido[4,5-d]pyrimidin-2-one (500 mg, 1.10 mmol, 65.46% yield) as a yellow solid

To a solution of 3-[6-[(4-methoxyphenyl)methyl]-6-azaspiro[3.5]nonan-9-yl]-1-methyl-7-methylsulfanyl-4H-pyrimido[4,5-d]pyrimidin-2-one (400 mg, 881.84 umol, 1 eq) in DCM (16 mL) was added TEA (89.23 mg, 881.84 umol, 122.74 uL, 1 eq) and 1-chloroethyl carbonochloridate (252.15 mg, 1.76 mmol, 2 eq) at 0° C.. The mixture was stirred at 20° C. for 12 hr. The reaction mixture was then concentrated under reduced pressure to remove solvent. Then MeOH(16 ml) was added to the mixture at 20° C. The mixture was stirred at 80° C. for 45 min. LCMS showed the reaction was completed and desired product was detected. The reaction mixture was concentrated under reduced pressure to give 3-(6-azaspiro[3.5]nonan-9-yl)-1-methyl-7-methylsulfanyl-4H-pyrimido[4,5-d]pyrimidin-2-one (290 mg, crude) as a yellow solid.

To a solution of 3-(6-azaspiro[3.5]nonan-9-yl)-1-methyl-7-methylsulfanyl-4H-pyrimido[4,5-d]pyrimidin-2-one (290 mg, 869.69 umol, 1 eq) in DCM (8 mL) was added TEA (440.02 mg, 4.35 mmol, 605.25 uL, 5 eq) and (Boc)₂0 (379.61 mg, 1.74 mmol, 399.59 uL, 2 eq). The mixture was stirred at 20° C. for 3 hr. LCMS showed the reaction was completed and desired product mass peak was detected. The reaction mixture was concentrated under reduced pressure. The residue was purified by flash silica gel chromatography (ISCO®; 12 g SepaFlash® Silica Flash Column, Eluent of 0-40% Ethyl acetate/Petroleum ether gradient @ 20 mL/min) to give tert-butyl 9-(1-methyl-7-methylsulfanyl-2-oxo-4H-pyrimido[4,5-d]pyrimidin-S-yl)-6-azaspiro[3.5]nonane-6-carboxylate (500 mg, 807.26 umol, 92.82% yield, 70% purity) as a white solid.

1H NMR (400 MHz, CHLOROFORM-d) δ ppm 8.07 (s, 1H) 4.23-4.47 (m, 5H) 3.43 (s, 3H) 2.70-2.86 (m, 2H) 2.57 (s, 3H) 1.76-1.93 (m, 6H) 1.65 (br d, J=4.25 Hz, 2H) 1.49 (s, 9 H)

To a solution of tert-butyl 9-(1-methyl-7-methylsulfanyl-2-oxo-4H-pyrimido[4,5-d]pyrimidin-S-yl)-6-azaspiro[3.5]nonane-6-carboxylate (500 mg, 1.15 mmol, 1 eq) in DCM (10 mL) was added m-CPBA (585.32 mg, 2.88 mmol, 85% purity, 2.5 eq) at 20° C. The mixture was stirred at 20° C. for 1.5 hr. LCMS showed the reaction was completed and desired product was detected. The reaction mixture was quenched with saturated Na₂SO₃ aqueous solution(15 mL) at 0° C., then the mixture was extracted with DCM (20 mL*3). The combined organic phase was dried with anhydrous Na₂SO₄, filtered and concentrated under vacuum. The residue was purified by flash silica gel chromatography (ISCO®; 12 g SepaFlash® Silica Flash Column, Eluent of 0-8% Ethyl acetate/Petroleum ether gradient @20 mL/min) to give tert-butyl 9-(1-methyl-7-methylsulfonyl-2-oxo-4H-pyrimido[4,5-d]pyrimidin-3-yl)-6-azaspiro[3.5]nonane-6-carboxylate (350 mg, 751.77 umol, 65.19% yield) as a white solid.

To a solution of tert-butyl 9-(1-methyl-7-methylsulfonyl-2-oxo-4H-pyrimido[4,5-d]pyrimidin-S-yl)-6-azaspiro[3.5]nonane-6-carboxylate (380 mg, 816.21 umol, 1 eq) and 4-(4-methylpiperazin-1-yl)aniline (468.36 mg, 2.45 mmol, 3 eq) in dioxane (15 mL) was added TFA (186.13 mg, 1.63 mmol, 120.87 uL, 2 eq) at 20° C. under N₂ atmosphere. The mixture was stirred at 120° C. for 12 hr under N₂ atmosphere. LCMS showed the reaction was completed and desired product was detected. The reaction mixture was concentrated under reduced pressure. The residue was dissolved in MeCN (4 mL) and filtered through a syringe filter. The filtrate was purified by prep-HPLC (FA condition) column: [Phenomenex Luna C18 75*30 mm*3 um;mobile phase: [water(FA)-ACN];B %: 1%-50%, 8 min] to give tert-butyl 9-[1-methyl-7-[4-(4-methylpiperazin-1-yl)anilino]-2-oxo-4H-pyrimido[4,5-d]pyrimidin-S-yl]-6-azaspiro[3.5]nonane-6-carboxylate (250 mg, 433.48 umol, 53.11% yield) as a white solid. 1H NMR (400 MHz, CHLOROFORM-d) δ ppm 7.95 (s, 1H) 7.53 (d, J=8.88 Hz, 2H) 6.95 (d, J=8.88 Hz, 2H) 4.12-4.51 (m, 5H) 3.28-3.52 (m, 7H) 3.05 (br s, 4H) 2.60-2.89 (m, 5H) 1.91 (br s, 3H) 1.74-1.85 (m, 2H) 1.54-1.73 (m, 3H) 1.49 (s, 9H)

To a solution of tert-butyl 9-[1-methyl-7-[4-(4-methylpiperazin-1-yl)anilino]-2-oxo-4H-pyrimido[4,5-d]pyrimidin-S-yl]-6-azaspiro[3.5]nonane-6-carboxylate (240 mg, 416.14 umol, 1 eq) in HCl/EtOAc (5 mL) (4M)was stirred at 20° C. for 1 hr. LCMS showed the reaction was completed and desired product was detected. The reaction mixture was concentrated under reduced pressure to give 3-(6-azaspiro[3.5]nonan-9-yl)-1-methyl-7-[4-(4-methylpiperazin-1-yl)anilino]-4H-pyrimido[4,5-d]pyrimidin-2-one (190 mg, crude, HCl) as a white solid.

To a solution of 3-(6-azaspiro[3.5]nonan-9-yl)-1-methyl-7-[4-(4-methylpiperazin-1-yl)anilino]-4H-pyrimido[4,5-d]pyrimidin-2-one (190 mg, 370.32 umol, 1 eq, HCl) in DCM(4 ML) was added DIPEA (143.58 mg, 1.11 mmol, 193.50 uL, 3 eq). Then, prop-2-enoyl chloride (33.52 mg, 370.32 umol, 30.20 uL, 1 eq) in DCM (1 ml) was added to the mixture under N₂ atmosphere at 0° C. The mixture was stirred 0° C. for 1 hr under N₂ atmosphere. LC-MS showed the desired compound was detected. The reaction mixture was concentrated under reduced pressure. The residue was purified by prep-HPLC (FA condition) [column: Phenomenex Luna C18 75*30 mm*3 um;mobile phase: [water(FA)-ACN];B %: 10%-50%, 8 min]to give 1-methyl-7-[4-(4-methylpiperazin-1-yl)anilino]-S-(6-prop-2-enoyl-6-azaspiro[3.5]nonan-9-yl)-4H-pyrimido[4,5-d]pyrimidin-2-one (21.21 mg, 35.99 umol, 9.72% yield, 97.86% purity, FA) as a pale yellow solid.

### Procedure for Preparation of Compound 099

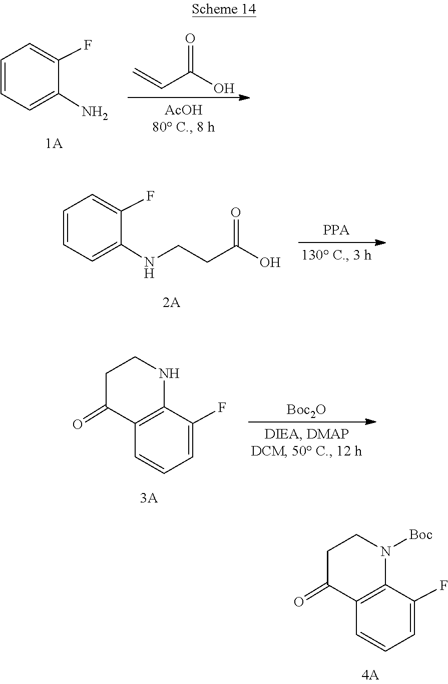

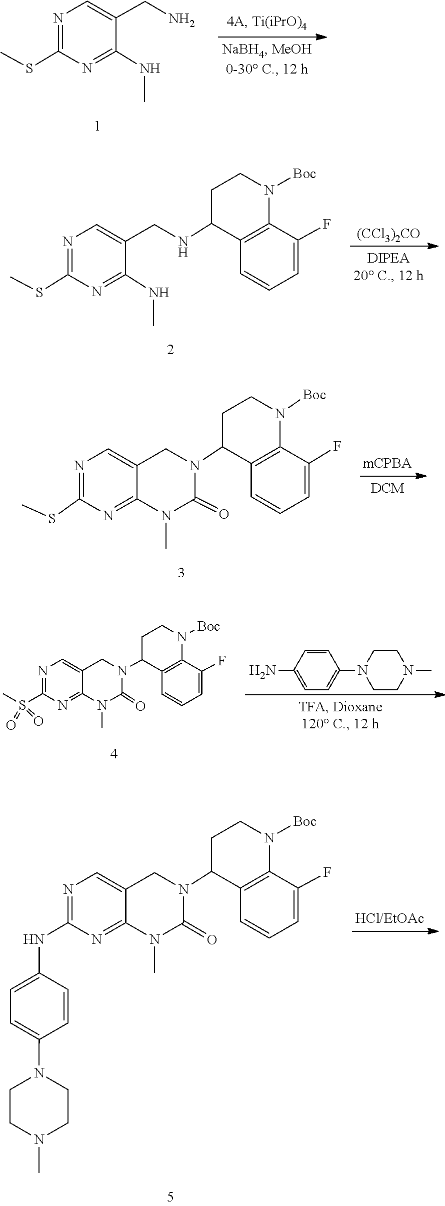

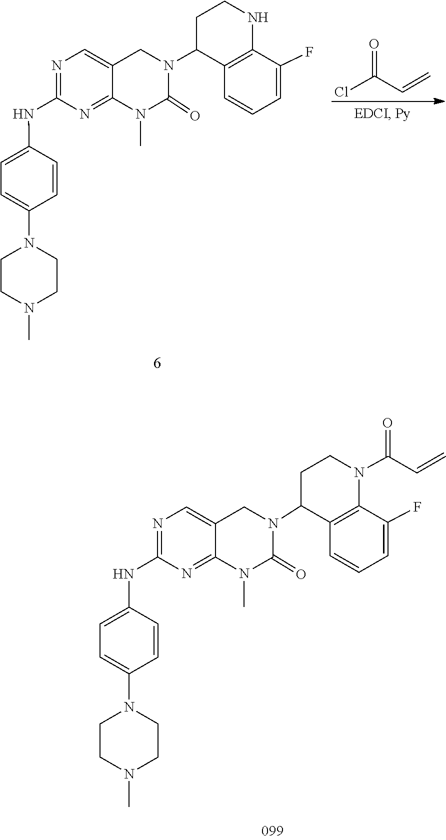

To a solution of 2-fluoroaniline (20 g, 179.99 mmol, 17.39 mL, 1 eq) and acrylic acid (12.97 g, 179.99 mmol, 12.35 mL, 1 eq) in AcOH (300 mL). The mixture was stirred at 80° C. for 8 hr. LCMS showed the desired product was detected. The reaction mixture was concentrated to give a residue. The residue was diluted with H₂O (200 mL) and extracted with EtOAc (200 mL×3). The combined organic layers were dried over Na2SO₄, filtered and the filtrate was concentrated to give a residue. The residue was purified by flash silica gel chromatography (ISCO®; 80 g SepaFlash® Silica Flash Column, Eluent of 0-10% Ethyl acetate/Petroleum ether gradient @ 100 mL/min) to give 3-(2-fluoroanilino)propanoic acid (26 g, 103.61 mmol, 57.57% yield, 73% purity) as a yellow solid.

To a solution of 3-(2-fluoroanilino)propanoic acid (16 g, 87.35 mmol, 1 eq) and PPA (160 g, 87.35 mmol, 1 eq). The mixture was stirred at 130° C. for 3 hr. LCMS showed the reaction was completed and desired product was detected. The reaction mixture was diluted with H₂O (1 L) and adjusted to pH=7 by NaOH solution. Then the mixture was extracted with dichloromethane (300 mL×3). The combined organic layers were dried over Na₂SO₄, filtered and the filtrate was concentrated to give 8-fluoro-2,3-dihydro-1 H-quinolin-4-one (7.96 g, crude) as a yellow solid. 1H NMR (400 MHz, DMSO-d6) 5=7.85 (d, J=8.0 Hz, 1H), 7.72-7.48 (m, 2H), 7.22-7.15 (m, 1H), 7.03-6.94 (m, 1H), 5.48 (br s, 1H), 3.94-3.83 (m, 2H), 3.04-2.95 (m, 2H)

To a solution of 8-fluoro-2,3-dihydro-1H-quinolin-4-one (7.7 g, 46.62 mmol, 1 eq) in DCM (250 mL) was added DMAP (569.55 mg, 4.66 mmol, 0.1 eq), (Boc)₂0 (12.21 g, 55.94 mmol, 12.85 mL, 1.2 eq) and DIEA (6.63 g, 51.28 mmol, 8.93 mL, 1.1 eq). The mixture was stirred at 50° C. for 12 hr. LCMS showed the reaction was completed and desired product was detected. The reaction mixture was quenched by HCl(1M) (200 ml)and extracted with Dichloromethane(150 mL×3). The combined organic phase was washed with brine (80 mL×1), dried over anhydrous Na2SO₄, filtered and concentrated give a residue. The residue was purified by flash silica gel chromatography (ISCO®; 25 g SepaFlash® Silica Flash Column, Eluent of 0-5% Ethyl acetate/Petroleum ether gradient @ 100 mL/min) to give tert-butyl 8-fluoro-4-oxo-2,3-dihydroquinoline-1-carboxylate (2.1 g, 3.72 mmol, 7.98% yield) as a yellow oil.

To a solution of tert-butyl 8-fluoro-4-oxo-2,3-dihydroquinoline-1-carboxylate (885.41 mg, 3.34 mmol, 1.5 eq) in MeOH (70 mL) was added 5-(aminomethyl)-N-methyl-2-methylsulfanyl-pyrimidin-4-amine (410 mg, 2.23 mmol, 1 eq) and tetraisopropoxytitanium (1.90 g, 6.68 mmol, 1.97 mL, 3 eq). The mixture was stirred at 30° C. for 12 hr. Then NaBH₄ (673.40 mg, 17.80 mmol, 8 eq) was added to the mixture at 0° C. The mixture was stirred at 30° C. for 3 hr. LCMS showed the desired product was detected. The residue was diluted with H₂O (70 mL) and filtered through a celite plug, the filtrate was extracted with EtOAc (60 mL×3). The combined organic layers were dried over Na2SO₄, filtered and the filtrate was concentrated to give a residue. The residue was purified by flash silica gel chromatography (ISCO®; 12 g SepaFlash® Silica Flash Column, Eluent of 0-22% Ethyl acetate/Petroleum ether gradient @ 100 mL/min) to give tert-butyl 8-fluoro-4-[[4-(methylamino)-2-methylsulfanyl-pyrimidin-5-yl]methylamino]-3,4-dihydro-2H-quinoline-1-carboxylate (100 mg, 207.59 umol, 9.33% yield, 90% purity) as a white oil.

To a solution of tert-butyl 8-fluoro-4-[[4-(methylamino)-2-methylsulfanyl-pyrimidin-5-yl]methylamino]-3,4-dihydro-2H-quinoline-1-carboxylate (100 mg, 230.66 umol, 1 eq) in DCM (4 mL) was added DIEA (149.05 mg, 1.15 mmol, 200.88 uL, 5 eq) and bis(trichloromethyl) carbonate (68.45 mg, 230.66 umol, 1 eq) at 0° C. The mixture was stirred at 20° C. for 12 hr. LCMS showed the reaction was completed and desired product was detected. The reaction mixture was quenched by saturated NaHCO₃solution (8 mL) and extracted with DCM (10 mL×3). The combined organic phase was washed with brine (10 mL×1), dried over anhydrous Na2SO₄, filtered and concentrated give a residue. The residue was purified by Prep-TLC to give tert-butyl 8-fluoro-4-(1-methyl-7-methylsulfanyl-2-oxo-4H-pyrimido[4,5-d]pyrimidin-S-yl)-3,4-dihydro-2H-quinoline-1-carboxylate (80 mg, 174.09 umol, 75.47% yield, 100% purity) as a white oil.

To a solution of tert-butyl 8-fluoro-4-(1-methyl-7-methylsulfanyl-2-oxo-4H-pyrimido[4,5-d]pyrimidin-S-yl)-3,4-dihydro-2H-quinoline-1-carboxylate (170 mg, 369.94 umol, 1 eq) in DCM (3 mL) was added MCPBA (150.22 mg, 739.87 umol, 85% purity, 2.0 eq) at 0° C. The mixture was stirred at 20° C. for 12 hr. LCMS showed the reaction was completed and desired product was detected. The reaction mixture was quenched by saturated Na2SO₃ solution (6 mL) and extracted with dichloromethane (6 mL×3). The combined organic phase was washed with NaHCO₃solution(5 mL×1), dried over anhydrous Na2SO₄, filtered and concentrated give a residue. The residue was purified by flash silica gel chromatography (ISCO®; 4 g SepaFlash® Silica Flash Column, Eluent of 0-32% Ethyl acetate/Petroleum ether gradient @ 100 mL/min) to give tert-butyl 8-fluoro-4-(1-methyl-7-methylsulfonyl-2-oxo-4H-pyrimido[4,5-d]pyrimidin-S-yl)-3,4-dihydro-2H-quinoline-1-carboxylate (40 mg, 62.66 umol, 16.94% yield, 77% purity) as a yellow solid.

To a solution of tert-butyl 8-fluoro-4-(1-methyl-7-methylsulfinyl-2-oxo-4H-pyrimido[4,5-d]pyrimidin-S-yl)-3,4-dihydro-2H-quinoline-1-carboxylate (71 mg, 149.31 umol, 1 eq) in dioxane (2 mL) was added TFA (25.54 mg, 223.96 umol, 16.58 uL, 1.5 eq) and 4-(4-methylpiperazin-1-yl)aniline (34.27 mg, 179.17 umol, 1.2 eq). The mixture was stirred at 120° C. for 12 hr. LCMS showed the desired product was detected. The reaction mixture was concentrated under reduced pressure. The residue was dissolved in ACN(1 mL), and the resulting solution was filtered through a syringe filter and then purified by prep-HPLC (FA condition; column: Phenomenex Luna C18 75*30 mm*3 um;mobile phase: [water(FA)-ACN];B %: 10%-50%, 8 min) to give tert-butyl 8-fluoro-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (50 mg, 76.32 umol, 51.12% yield, 92% purity) as a white solid.

A solution of tert-butyl 8-fluoro-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (50 mg, 82.96 umol, 1 eq) in HCl/EtOAc (3 mL) (4M) was stirred at 20° C. for 1 hr. LCMS showed the reaction was completed and desired product was detected. The reaction mixture was concentrated under reduced pressure to give 3-(8-fluoro-1,2,3,4-tetrahydroquinolin-4-yl)-1-methyl-7-[4-(4-methylpiperazin-1-yl)anilino]-4H-pyrimido[4,5-d]pyrimidin-2-one (44 mg, crude, HCl) as a white solid.

To a solution of 3-(8-fluoro-1,2,3,4-tetrahydroquinolin-4-yl)-1-methyl-7-[4-(4-methylpiperazin-1-yl)anilino]-4H-pyrimido[4,5-d]pyrimidin-2-one (10 mg, 18.55 umol, 1 eq, HCl) and acrylic acid (4.02 mg, 55.65 umol, 3.81 uL, 3 eq) in Pyridine (0.5 mL) was added EDCI (5.33 mg, 27.83 umol, 1.5 eq) at 20° C. The mixture was stirred at 50° C. for 12 hr under N₂ atmosphere. LCMS showed the reaction was completed and desired mass peak was detected. The mixture was concentrated under vacuum. The residue was diluted with 2 mL H₂O and extracted with EtOAc (3 mL*3). The combined organic phase was dried with anhydrous Na₂SO₄, the mixture was filtered and the filtrate was concentrated under vacuum. The residue was purified by prep-HPLC (neutral condition, column: Waters Xbridge Prep OBD C18 150*40 mm*10 um;mobile phase: [water(NH₄HCO₃)-ACN];B %: 35%-55%, 8 min) to give 3-(8-fluoro-1-prop-2-enoyl-3,4-dihydro-2H-quinolin-4-yl)-1-methyl-7-[4-(4-methylpiperazin-1-yl)anilino]-4H-pyrimido[4,5-d]pyrimidin-2-one (5.17 mg, 94.26% purity) as a pale yellow solid.

### Procedure for Preparation of Compound 100

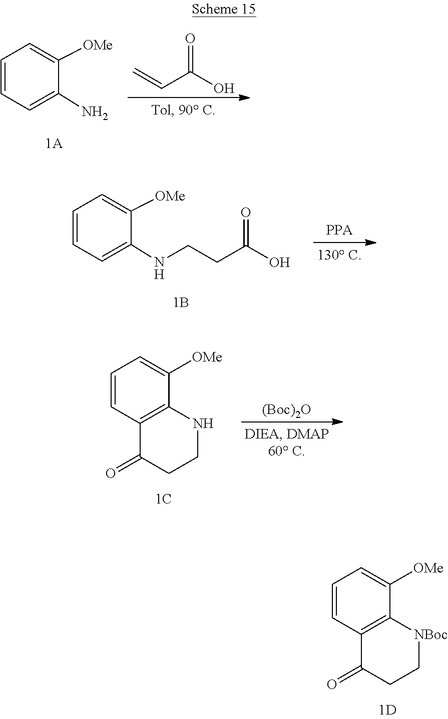

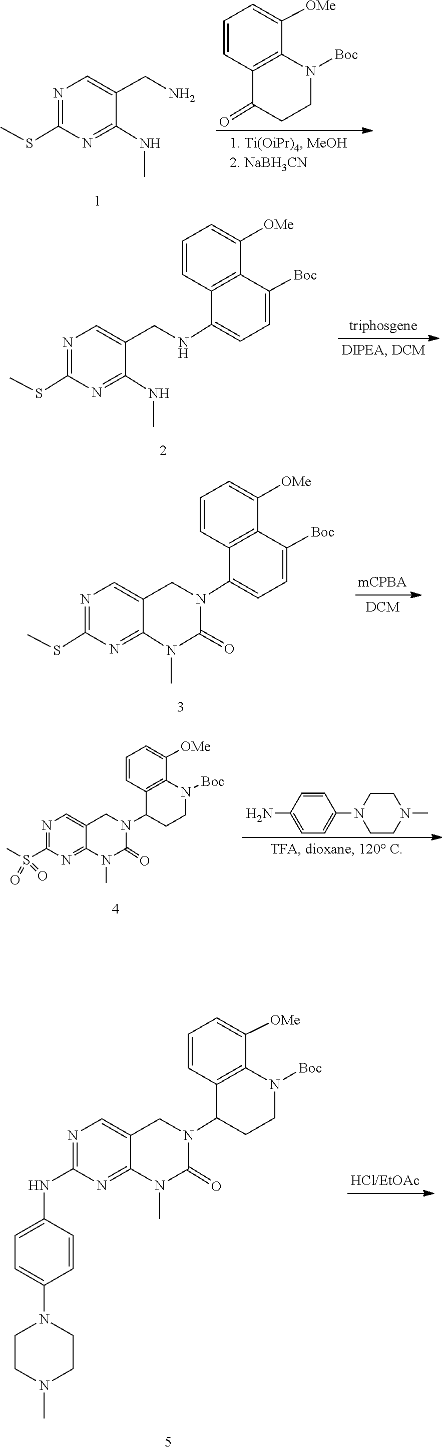

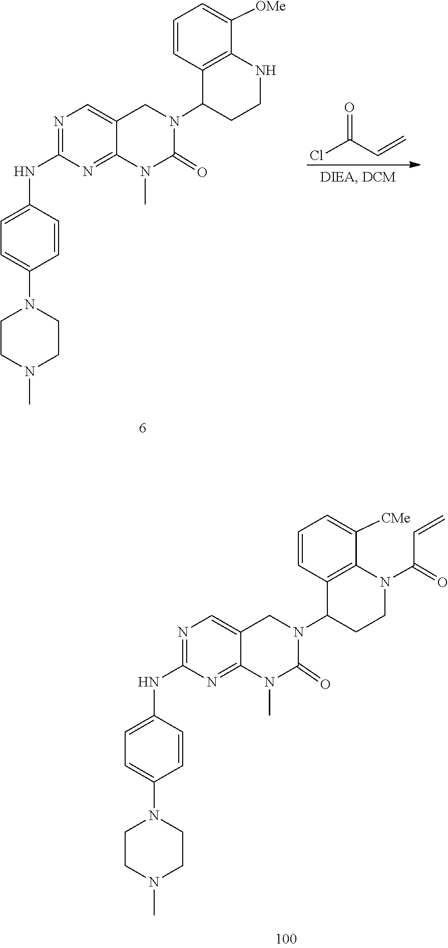

To a solution of 2-methoxyaniline (15 g, 121.8 mmol, 13.7 mL, 1 eq) in Tol. (80 mL) was added acrylic acid (8.7 g, 121.8 mmol, 8.4 mL, 1 eq). The reaction mixture was stirred at 90° C. for 12 hr. LCMS showed consumption of reactant and formation of the desired product mass. The reaction mixture was concentrated under reduced pressure to give a residue. The residue was purified by flash silica gel chromatography (12 g Silica Flash Column, Eluent of 0-100% Ethyl acetate/Petroleum ether gradient @ 100 mL/min) to yield 3-(2-methoxyanilino)propanoic acid (15 g, 76.8 mmol, 63.0% yield) obtained as a yellow solid.

A mixture of 3-(2-methoxyanilino)propanoic acid (7.5 g, 38.4 mmol, 1 eq) in PPA (80 g) was stirred at 130° C. for 3 hr. The reaction was monitored by LCMS which showed the reactant was consumed completely and the desired mass peak was detected. The reaction mixture was diluted with H₂O (200 mL). And then the pH of the reaction mixture was adjusted by addition NaOH (4 M) to 7. After that, the mixture was extracted with dichloromethane (500 mL×3). The combined organic layers were dried over Na₂SO₄, filtered and concentrated under reduced pressure. The residue was purified by flash silica gel chromatography (40 g Silica Flash Column, Eluent of 0-100% Ethyl acetate/Petroleum ether gradient @ 100 mL/min) to give 8-methoxy-2, 3-dihydro-1 H-quinolin-4-one (6 g) as a red solid.

To a solution of 8-methoxy-2,3-dihydro-1 H-quinolin-4-one (5 g, 28.2 mmol, 1 eq) was added Boc₂O (123.1 g, 564.3 mmol, 129.6 mL, 20 eq), DMAP (344.7 mg, 2.8 mmol, 0.1 eq) and DIEA (7.2 g, 56.4 mmol, 9.82 mL, 2 eq). The reaction mixture was stirred at 60° C. for 3 hr. The reaction was monitored by LCMS which showed complete consumption of reactant and detection of the desired mass peak. The reaction mixture was concentrated under reduced pressure to give the residue. The residue was purified by flash silica gel chromatography (40 g Silica Flash Column, Eluent of 0-100% Ethyl acetate/Petroleum ether gradient @ 100 mL/min) to give 8-methoxy-2,3-dihydro-1H-quinolin-4-one (4 g, 22.5 mmol, 80.0% yield) as a white solid.

To a solution of tert-butyl 8-methoxy-4-oxo-2,3-dihydroquinoline-1-carboxylate (830 mg, 2.9 mmol, 1 eq) in MeOH (30 mL) was added 5-(aminomethyl)-N-methyl-2-methylsulfanyl-pyrimidin-4-amine (882.3 mg, 4.7 mmol, 1.6 eq), Ti(i-PrO)₄ (1.2 g, 4.4 mmol, 1.3 mL, 1.5 eq) and AcOH (1.0 g, 17.9 mmol, 1.0 mL, 6 eq) was added to the mixture to adjust pH=5 and the mixture was stirred at 25° C. for 2 hr. NaBH₃CN (564.2 mg, 8.9 mmol, 3 eq) was added to the mixture. The mixture was stirred at 65° C. for 12 hr. The reaction was monitored by LCMS which showed complete consumption of reactant and detection of the desired mass peak. The reaction mixture was concentrated under reduced pressure to remove solvent. The residue was diluted with NaHCO₃ 30 mL and extracted with dichloromethane (30 mL×3). The combined organic layers were washed with brine (30 mL×3), dried over Na₂SO₄, filtered and concentrated under reduced pressure to give a residue.

The residue was purified by flash silica gel chromatography (12 g Silica Flash Column, Eluent of 0-100% Ethyl acetate/Petroleum ether gradient @ 100 mL/min) to yield the product tert-butyl 8-methoxy-4-[[4-(methylamino)-2-methylsulfanyl-pyrimidin-5-yl]methylamino]-3,4-dihydro-2H-quinoline-1-carboxylate (600 mg, 1.35 mmol, 44.99% yield) as a yellow solid.

To a solution of tert-butyl 8-methoxy-4-[[4-(methylamino)-2-methylsulfanyl-pyrimidin-5-yl]methylamino]-3,4-dihydro-2H-quinoline-1-carboxylate (500 mg, 1.1 mmol, 1 eq) in DCM (8 mL) was added DIEA (435.0 mg, 3.3 mmol, 586.3 uL, 3 eq) and bis(trichloromethyl) carbonate (332.9 mg, 1.1 mmol, 1 eq) at 0° C. The mixture was stirred at 25° C. for 12 hr.

The reaction was monitored by LCMS which showed complete consumption of reactant and detection of the desired product. The residue was diluted with NaHCO₃ 15 mL and extracted with dichloromethane (20 mL×3). The combined organic layers were washed with brine (15 mL×1), dried over Na₂SO₄, filtered and concentrated under reduced pressure to give tert-butyl 8-methoxy-4-(1-methyl-7-methylsulfanyl-2-oxo-4H-pyrimido[4,5-d] pyrimidin-S-yl)-3,4-dihydro-2H-quinoline-1-carboxylate (550 mg, crude) as a yellow oil.

To a solution of tert-butyl 8-methoxy-4-(1-methyl-7-methylsulfanyl-2-oxo-4H-pyrimido[4,5-d]pyrimidin-S-yl)-3,4-dihydro-2H-quinoline-1-carboxylate (450 mg, 954.2 umol, 1 eq) in DCM (8 mL) was added m-CPBA (387.4 mg, 1.9 mmol, 85% purity, 2 eq) at 0° C.

The mixture was stirred at 25° C. for 12 hr. The reaction was monitored by LCMS which showed complete consumption of reactant and detection of the desired product. The residue was diluted with Na₂SO₃ (10%) 10 mL and extracted with dichloromethane (20 mL×3). The combined organic layers were washed with NaHCO₃ (20 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 120 mL/min). to give tert-butyl 8-methoxy-4-(1-methyl-7-methylsulfonyl-2-oxo-4H-pyrimido[4,5-d]pyrimidin-S-yl)-3,4-dihydro-2H-quinoline-1-carboxylate (350 mg, 695.0 umol, 72.8% yield) as a white solid.

4-(4-methylpiperazin-1-yl)aniline (45.5 mg, 238.3 umol, 1.2 eq) in dioxane (2 mL) was added tert-butyl 8-methoxy-4-(1-methyl-7-methylsulfonyl-2-oxo-4H-pyrimido[4,5-d]pyrimidin-3-yl)-3,4-dihydro-2H-quinoline-1-carboxylate (100 mg, 198.5 umol, 1 eq) and TFA (33.9 mg, 297.8 umol, 22.0 uL, 1.5 eq). The reaction mixture was stirred at 120° C. for 12 hr. The reaction was monitored by LCMS which showed complete consumption of reactant and detection of the desired product. The reaction mixture was diluted with dimethyl sulfoxide 3 mL and filtered. The residue was purified by prep-HPLC (FA condition; column: Phenomenex Luna C18 75*30 mm*3 um;mobile phase: [water(FA)-ACN];B %: 15%-45%, 8 min) to give tert-butyl 8-methoxy-4-[1-methyl-7-[4-(4-methylpiperazin-1-yl) anilino]-2-oxo-4H-pyrimido[4,5-d]pyrimidin-S-yl]-3,4-dihydro-2H-quinoline-1-carboxylate (160 mg, 260.2 umol, 43.6% yield) as a white solid.

A solution of tert-butyl 8-methoxy-4-[1-methyl-7-[4-(4-methylpiperazin-1-yl)anilino]-2-oxo-4H-pyrimido[4,5-d] pyrimidin-S-yl]-3,4-dihydro-2H-quinoline-1-carboxylate (60 mg, 97.6 umol, 1 eq) in HCl/EtOAc (2 mL, 4 M) was stirred at 25° C. for 10 min. The reaction was monitored by LCMS which showed complete consumption of reactant and detection of the desired product. The reaction mixture was concentrated under reduced pressure to give the crude product 3-(8-methoxy-1, 2, 3, 4-tetrahydroquinolin-4-yl)-1-methyl-7-[4-(4-methylpiperazin-1-yl) anilino]-4H-pyrimido [4, 5-d] pyrimidin-2-one (60 mg, crude) as yellow solid and was used into the next step without further purification.

To a solution of 3-(8-methoxy-1,2,3,4-tetrahydroquinolin-4-yl)-1-methyl-7-[4-(4-methylpiperazin-1-yl)anilino]-4H-pyrimido[4,5-d]pyrimidin-2-one (30 mg, 58.3 umol, 1 eq) in DCM (2 mL) was added DIEA (22.6 mg, 174.8 umol, 30.4 uL, 3 eq) and prop-2-enoyl chloride (6.3 mg, 69.9 umol, 5.7 uL, 1.2 eq) at 0° C. The mixture was stirred at 25° C. for 1 hr. The reaction was monitored by LCMS which showed complete consumption of reactant and detection of the desired mass peak. The residue was purified by prep-HPLC (FA condition; column: Phenomenex Luna 80*30 mm*3 um;mobile phase: [water(FA)-ACN];B %: 1%-30%, 8 min) to yield 3-(8-methoxy-1-prop-2-enoyl-3,4-dihydro-2H-quinolin-4-yl)-1-methyl-7-[4-(4-methylpiperazin-1-yl)anilino]-4H-pyrimido[4,5-d]pyrimidin-2-one (15 mg, 24.4 umol, 20.9% yield, 100% purity, FA) as a white solid. MS (ESI): m/z=569.2 [M+H]⁺

### Procedure for Preparation of Compound 102

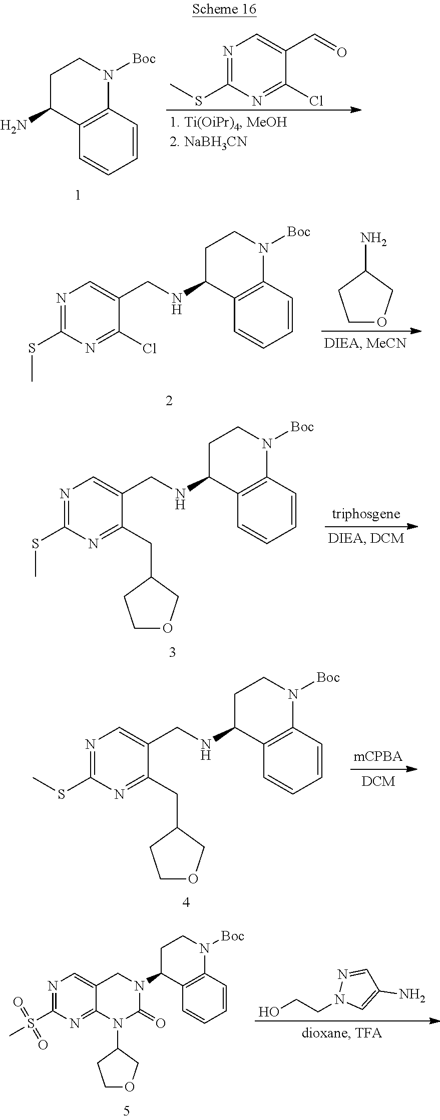

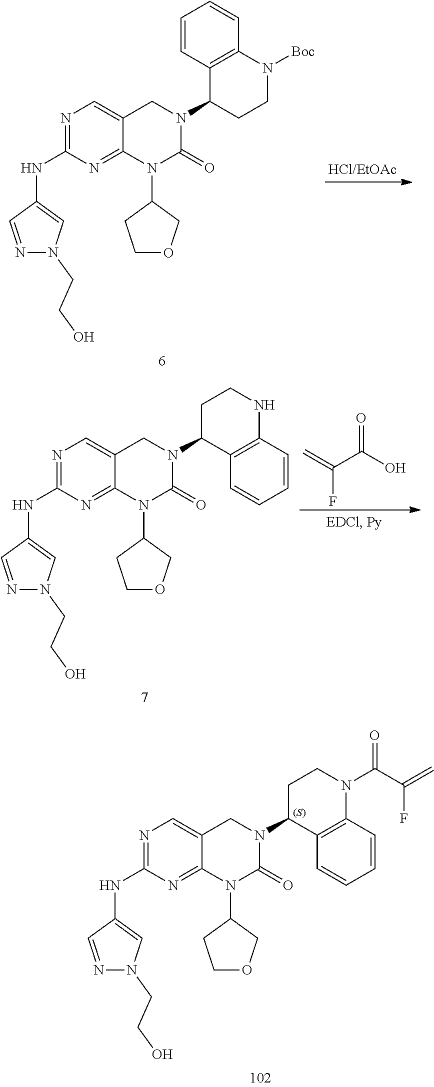

To a solution of Ti(Oi-Pr)₄ (3.49 g, 12.0 mmol, 3.5 mL, 3.0 eq) and (S)-tert-butyl 4-amino-3,4-dihydroquinoline-1(2H)-carboxylate (1.0 g, 4.0 mmol, 1.0 eq) in MeOH (15 mL) was added 4-chloro-2-(methylthio)pyrimidine-5-carbaldehyde (759.6 mg, 4.0 mmol, 1.0 eq). The mixture was stirred at 30° C. for 12 hr. NaBH₃CN (759.1 mg, 12.0 mmol, 3.0 eq) was added to the mixture. The mixture was stirred at 25° C. for 3 hr. LC-MS showed desired mass was detected. The reaction mixture was concentrated under reduced pressure to remove MeOH. The reaction mixture was quenched by addition NH₄Cl 50 mL at 0° C. The reaction mixture was extracted with EtOAc 60 mL (20 mL×3), dried over Na₂SO₄, filtered and concentrated under reduced pressure to give a residue. The residue was purified by flash silica gel chromatography (Silica Flash Column, Eluent of 0˜30% Ethyl acetate/Petroleum ether gradient @ 100 mL/min) to give (3)-tert-butyl 4-(((4-chloro-2-(methylthio)pyrimidin-5-yl)methyl)amino)-3,4-dihydroquinoline-1(2H)-carboxylate (1.1 g, 2.08 mmol, 51.65% yield, 79.6% purity) as a colorless oil.

¹H NMR (400 MHz, CHLOROFORM-d) δ 8.57-8.52 (m, 1H), 7.75 (d, J=8.4 Hz, 1H), 7.28 (br s, 1H), 7.25-7.19 (m, 1H), 7.07-7.01 (m, 1H), 4.75 (br s, 1H), 3.93-3.87 (m, 1H), 3.87-3.73 (m, 2H), 3.71 (s, 1H), 2.60-2.56 (m, 3H), 2.11-1.93 (m, 2H), 1.54 (s, 8H).

To a solution of tetrahydrofuran-S-amine (387.5 mg, 3.1 mmol, 1.2 eq, HCl) in MeCN (12 mL) was added DIEA (1.0 g, 7.8 mmol, 1.3 mL, 3.0 eq) and (S)-tert-butyl 4-(((4-chloro-2-(methylthio)pyrimidin-5-yl)methyl)amino)-3,4-dihydroquinoline-1 (2H)-carboxylate (1.1 g, 2.6 mmol, 1.0 eq). The mixture was stirred at 70° C. for 12 hr. LC-MS showed desired mass was detected. The residue was diluted with H₂O 60 mL and extracted with EtOAc 90 mL (30 mL×3). The combined organic layers were washed with brine 90 mL (30 mL×3), dried over Na₂SO₄, filtered and concentrated under reduced pressure to give a residue. The residue was purified by flash silica gel chromatography (Silica Flash Column, Eluent of 0-30% Ethyl acetate/Petroleum ether gradient @ 100 mL/min) to give (4S)-tert-butyl 4-(((2-(methylthio)-4-((tetrahydrofuran-S-yl)amino)pyrimidin-5-yl)methyl)amino)-3,4-dihydroquinoline-1(2H)-carboxylate (386 mg, 808.64 umol, 30.95% yield, 98.8% purity) as a white solid.

¹H NMR (400 MHz, CHLOROFORM-d) δ 7.83-7.75 (m, 2H), 7.57-7.48 (m, 1H), 7.26-7.18 (m, 2H), 7.09-7.01 (m, 1H), 4.74-4.64 (m, 1H), 4.04-3.92 (m, 2H), 3.91-3.82 (m, 3H), 3.81-3.76 (m, 2H), 3.76-3.62 (m, 3H), 2.52 (s, 3H), 2.39-2.27 (m, 1H), 2.14-2.03 (m, 2H), 2.00-1.80 (m, 3H), 1.57-1.53 (m, 9H).

To a solution of (4S)-tert-butyl 4-(((2-(methylthio)-4-((tetrahydrofuran-S-yl)amino)pyrimidin-5-yl)methyl)amino)-3,4-dihydroquinoline-1(2H)-carboxylate (386.0 mg, 818.4 umol, 1.0 eq) in DCM (3 mL) was added DIEA (317.3 mg, 2.4 mmol, 427.6 uL, 3.0 eq) and bis(trichloromethyl) carbonate (218.5 mg, 736.6 umol, 0.9 eq) at 0° C. The mixture was stirred at 20° C. for 12 hr. LC-MS showed desired mass was detected. The residue was diluted with NaHCO₃ 5 mL and extracted with DCM 30 mL (10 mL×3). The combined organic layers were washed with brine 20 mL (10 mL×2), dried over Na₂SO₄, filtered and concentrated under reduced pressure to give (4S)-tert-butyl 4-(7-(methylthio)-2-oxo-1-(tetrahydrofuran-S-yl)-1,2-dihydropyrimido[4,5-d]pyrimidin-S(4H)-yl)-3,4-dihydroquinoline-1(2H)-carboxylate (480 mg, crude) as yellow oil.

To a solution of (4S)-tert-butyl 4-(7-(methylthio)-2-oxo-1-(tetrahydrofuran-S-yl)-1,2-dihydropyrimido[4,5-d]pyrimidin-S(4H)-yl)-3,4-dihydroquinoline-1(2H)-carboxylate (400 mg, 803.8 umol, 1.0 eq) in DCM (4 mL) was added m-CPBA (326.4 mg, 1.6 mmol, 85% purity, 2.0 eq) at 0° C. The mixture was stirred at 25° C. for 12 hr. LC-MS showed desired mass was detected. The reaction was quenched by addition of Na₂SO₃ solution (5 mL) and the mixture was extracted with DCM (5×3 mL). The organic phase was separated, washed with NaHCO₃ (5 mL×3), dried over Na₂SO₄, filtered and concentrated under reduced pressure to give a residue. The residue was purified by flash silica gel chromatography (Silica Flash Column, Eluent of 0-100% Ethyl acetate/Petroleum ether gradient @ 100 mL/min) to give (4S)-tert-butyl 4-(7-(methylsulfonyl)-2-oxo-1-(tetrahydrofuran-S-yl)-1,2-dihydropyrimido[4,5-d]pyrimidin-S(4H)-yl)-3,4-dihydroquinoline-1(2H)-carboxylate (350 mg, 581.56 umol, 72.35% yield, 88% purity) as a yellow solid.

To a solution of (4S)-tert-butyl 4-(7-(methylsulfonyl)-2-oxo-1-(tetrahydrofuran-S-yl)-1,2-dihydropyrimido[4,5-d]pyrimidin-S(4H)-yl)-3,4-dihydroquinoline-1(2H)-carboxylate (310 mg, 585.3 umol, 1.0 eq) in dioxane (2 mL) was added 2-(4-aminopyrazol-1-yl)ethanol (111.6 mg, 878.0 umol, 1.5 eq) and TFA (133.4 mg, 1.1 mmol, 86.6 uL, 2.0 eq). The mixture was stirred at 80° C. for 12 hr. LC-MS showed desired mass was detected. The reaction mixture was concentrated under reduced pressure to remove solvent. The residue was purified by prep-HPLC (FA condition, column: Phenomenex Luna C18 75*30 mm*3 um;mobile phase: [water(FA)-ACN];B %: 15%-55%, 8 min) to give (4R)-tert-butyl 4-(7-((1-(2-hydroxyethyl)-1H-pyrazol-4-yl)amino)-2-oxo-1-(tetrahydrofuran-S-yl)-1,2-dihydropyrimido[4,5-d]pyrimidin-3(4H)-yl)-3,4-dihydroquinoline-1(2H)-carboxylate (110 mg, 190.76 umol, 32.59% yield) as a yellow solid.

A mixture of (4R)-tert-butyl 4-(7-((1-(2-hydroxyethyl)-1H-pyrazol-4-yl)amino)-2-oxo-1-(tetrahydrofuran-S-yl)-1,2-dihydropyrimido[4,5-d]pyrimidin-S(4H)-yl)-3,4-dihydroquinoline-1(2H)-carboxylate (100 mg, 173.4 umol, 1.0 eq) in HCl/EtOAc (2 mL, 4 M) was stirred at 25° C. for 1 hr. LC-MS showed desired mass was detected. The reaction mixture was concentrated under reduced pressure to give 7-((1-(2-hydroxyethyl)-1H-pyrazol-4-yl)amino)-1-(tetrahydrofuran-S-yl)-S—((S)-1,2,3,4-tetrahydroquinolin-4-yl)-3,4-dihydropyrimido[4,5-d]pyrimidin-2(1H)-one (123 mg, crude, HCl salt) as a yellow solid.

To a solution of 7-((1-(2-hydroxyethyl)-1 H-pyrazol-4-yl)amino)-1-(tetrahydrofuran-S-yl)-S—((S)-1,2,3,4-tetrahydroquinolin-4-yl)-3,4-dihydropyrimido[4,5-d]pyrimidin-2(1H)-one (80 mg, 167.8 umol, 1.0 eq) in Pyridine (1 mL) was added 2-fluoroacrylic acid (18.1 mg, 201.4 umol, 1.2 eq) and EDCI (48.2 mg, 251.8 umol, 1.5 eq). The mixture was stirred at 50° C. for 12 hr. LC-MS showed desired mass was detected. The reaction mixture was concentrated under reduced pressure to remove solvent. The residue was purified by prep-HPLC (FA condition, column: Phenomenex Luna C18 75*30 mm*3 um;mobile phase: [water(FA)-ACN];B %: 1%-40%, 8 min) to give 3-((S)-1-(2-fluoroacryloyl)-1,2,3,4-tetrahydroquinolin-4-yl)-7-((1-(2-hydroxyethyl)-1 H-pyrazol-4-yl)amino)-1-(tetrahydrofuran-S-yl)-3,4-dihydropyrimido[4,5-d]pyrimidin-2(1H)-one (7 mg, 12.4 umol, 7.3% yield, 97.2% purity) as a yellow solid.

### Procedure for Preparation of Compound 106

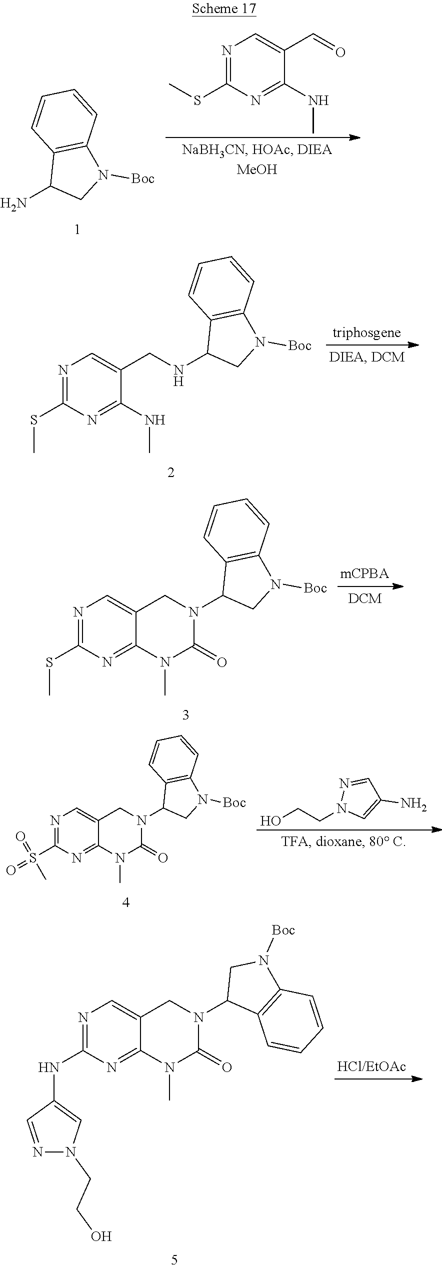

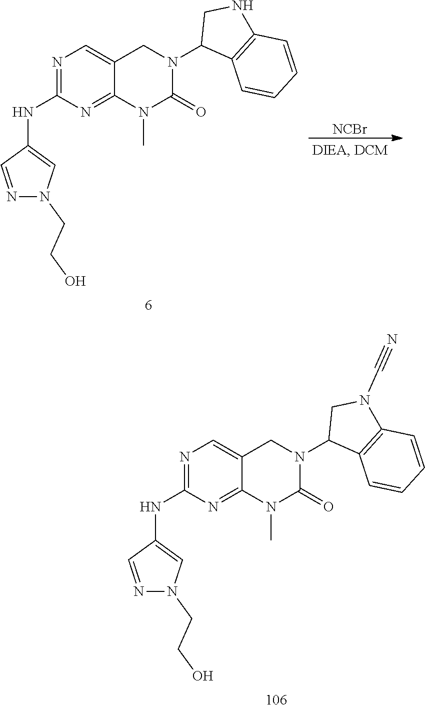

To a solution of tert-butyl 3-aminoindoline-1-carboxylate (350 mg, 1.5 mmol, 1 eq) in MeOH (8 mL) was added DIEA (482.6 mg, 3.7 mmol, 650.5 uL, 2.5 eq), 4-(methylamino)-2-methylsulfanyl-pyrimidine-5-carbaldehyde (301.0 mg, 1.6 mmol, 1.1 eq), AcOH (538.2 mg, 8.9 mmol, 512.6 uL, 6 eq) and NaBH₃CN (563.2 mg, 8.9 mmol, 6 eq). The reaction mixture was stirred at 25° C. for 12 hr. LCMS showed consumption of reactant and formation of the desired product mass. The residue was diluted with NaHCO₃ 15 mL and extracted with DCM (20 mL×3). The combined organic layers were dried over Na₂SO₄, filtered and concentrated under reduced pressure to give a residue. The residue was purified by flash silica gel chromatography (12 g Silica Flash Column, Eluent of 0-12% Ethyl acetate/Petroleum ether gradient @ 80 mL/min) to yield tert-butyl 3-[[4-(methylamino)-2-methylsulfanyl-pyrimidin-5-yl]methylamino] indoline-1-carboxylate (500 mg, 1.2 mmol, 83.3% yield) as a white solid.

To a solution of tert-butyl 3-[[4-(methylamino)-2-methylsulfanyl-pyrimidin-5-yl]methylamino]indoline-1-carboxylate (500 mg, 1.2 mmol, 1 eq) in DCM (10 mL) was added bis(trichloromethyl) carbonate (295.6 mg, 996.2 umol, 0.8 eq) and DIEA (482.8 mg, 3.7 mmol, 650.7 uL, 3 eq) at 0° C. The reaction mixture was stirred at 25° C. for 12 hr. The reaction was monitored by LCMS which showed the reactant was consumed completely and the desired mass peak was detected. The residue was diluted with NaHCO₃ (15 mL) and extracted with dichloromethane (20 mL×3). The combined organic layers were washed with brine (15 mL×1), dried over Na₂SO₄, filtered and concentrated under reduced pressure to give the crude product tert-butyl 3-(1-methyl-7-methylsulfanyl-2-oxo-4H-pyrimido[4,5-d]pyrimidin-S-yl)indoline-1-carboxylate (650 mg, crude) as yellow oil.

To a solution of tert-butyl 3-(1-methyl-7-methylsulfanyl-2-oxo-4H-pyrimido[4,5-d]pyrimidin-S-yl)indoline-1-carboxylate (650 mg, 1.5 mmol, 1 eq) in DCM (15 mL) was added m-CPBA (555.6 mg, 2.7 mmol, 85% purity, 1.8 eq) at 0° C. The mixture was stirred at 25° C. for 12 hr. The reaction was monitored by LCMS which showed complete consumption of reactant and detection of the desired mass peak. The reaction mixture was quenched by addition Na₂SO₃ (15 mL) at 0° C., and then extracted with dichloromethane (15 mL×3). The combined organic layers were dried over Na₂SO₄, filtered and concentrated under reduced pressure to give the product ttert-butyl 3-(1-methyl-7-methylsulfonyl-2-oxo-4H-pyrimido[4,5-d]pyrimidin-S-yl)indoline-1-carboxylate (700 mg, crude) as a white solid.

2-(4-aminopyrazol-1-yl)ethanol (17.9 mg, 141.4 umol, 1.3 eq) in dioxane (2 mL) was added tert-butyl 3-(1-methyl-7-methylsulfonyl-2-oxo-4H-pyrimido[4,5-d]pyrimidin-S-yl)indoline-1-carboxylate (50 mg, 108.8 umol, 1 eq) and TFA (18.6 mg, 163.1 umol, 12.0 uL, 1.5 eq). The reaction mixture was stirred at 80° C. for 12 hr. The reaction was monitored by LCMS which showed complete consumption of reactant and detection of the desired product. The residue was purified by prep-HPLC (FA condition; column: Phenomenex Luna C18 200*40 mm*10 um; mobile phase: [water(FA)-ACN];B %: 30%-60%, 8 min) to give tert-butyl 3-[7-[[1-(2-hydroxyethyl)pyrazol-4-yl] amino]-1-methyl-2-oxo-4H-pyrimido[4,5-d]pyrimidin-S-yl]indoline-1-carboxylate (300 mg) as a white solid.

A mixture of tert-butyl 3-[7-[[1-(2-hydroxyethyl) pyrazol-4-yl] amino]-1-methyl-2-oxo-4H-pyrimido [4, 5-d] pyrimidin-S-yl] indoline-1-carboxylate (120 mg, 236.8 umol, 1 eq) in HCl/EtOAc (2 mL, 4 M) was stirred at 25° C. for 10 min. The reaction was monitored by LCMS which showed complete consumption of reactant and detection of the desired product. The reaction mixture was concentrated under reduced pressure to give 7-[[1-(2-hydroxyethyl) pyrazol-4-yl] amino]-S-indolin-S-yl-1-methyl-4H-pyrimido [4, 5-d] pyrimidin-2-one (150 mg, crude, HCl salt) as yellow solid.

To a solution of 7-[[1-(2-hydroxyethyl)pyrazol-4-yl]amino]-S-indolin-S-yl-1-methyl-4H-pyrimido[4,5-d]pyrimidin-2-one (50 mg, 112.8 umol, 1 eq, HCl) in DCM (2 mL) was added DIEA (18.9 mg, 146.7 umol, 25.5 uL, 1.3 eq) and bromocyanide (23.9 mg, 225.7 umol, 16.6 uL, 2 eq). The mixture was stirred at 20° C. for 12 hr. The reaction was monitored by LCMS which showed complete consumption of reactant and detection of the desired mass peak. The residue was purified by prep-HPLC (FA condition; column: Phenomenex Luna C18 200*40 mm*10 um;mobile phase: [water(FA)-ACN];B %: 10%-40%, 8 min) to yield 3-[7-[[1-(2-hydroxyethyl) pyrazol-4-yl]amino]-1-methyl-2-oxo-4H-pyrimido[4,5-d]pyrimidin-S-yl]indoline-1-carbonitrile (92.804% purity, 15 mg) as a white solid. MS (ESI): m/z=432.1 [M+H]⁺

### Procedure for Preparation of Compound 107

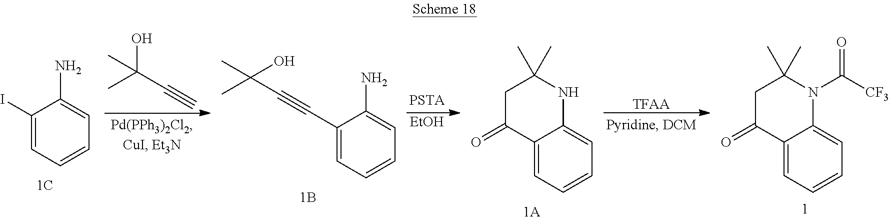

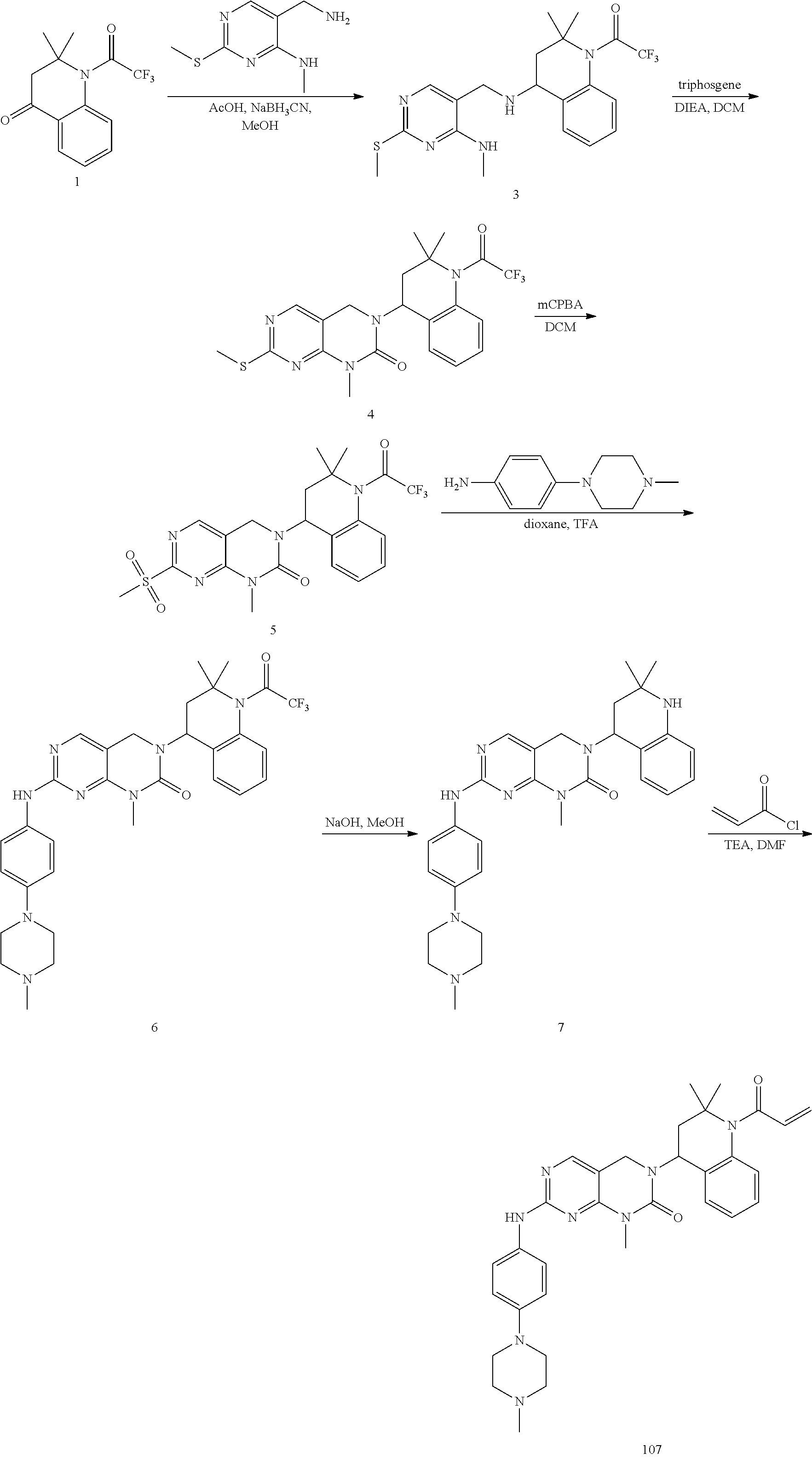

To a solution of 2-iodoaniline (10.0 g, 45.6 mmol, 1.0 eq) and 2-methylbut-3-yn-2-ol (4.6 g, 54.7 mmol, 5.3 mL, 1.2 eq) in TEA (140 mL) was added Pd(PPh₃)₂Cl₂ (640.9 mg, 913.1 umol, 0.02 eq) and CuI (173.9 mg, 913.1 umol, 0.02 eq). The mixture was degassed and purged with N₂ for 3 times, and then the mixture was stirred at 50° C. for 12 hr. LC-MS showed desired mass was detected. The reaction mixture was diluted with H₂O 100 mL and extracted with EtOAc (100 mL×3). The combined organic layers were washed with brine (100 mL×3), dried over Na₂SO₄, filtered and concentrated under reduced pressure to give a residue. The residue was purified by flash silica gel chromatography (Silica Flash Column, Eluent of 0-30% Ethyl acetate/Petroleum ether gradient @ 100 mL/min) to give 4-(2-aminophenyl)-2-methylbut-3-yn-2-ol (5.2 g, 26.92 mmol, 58.95% yield, 90.7% purity) as brown oil.

¹H NMR (400 MHz, CHLOROFORM-d) δ 7.21-7.15 (m, 1H), 7.08-7.00 (m, 1H), 6.64-6.56 (m, 2H), 4.05 (br d, J=7.1 Hz, 2H), 2.24-1.82 (m, 1H), 1.57 (s, 6H).

To a solution of 4-(2-aminophenyl)-2-methylbut-3-yn-2-ol (5.2 g, 29.6 mmol, 1.0 eq) in EtOH (50 mL) was added PTSA (7.1 g, 41.5 mmol, 1.4 eq). The mixture was stirred at 80° C. for 10 hr. LC-MS showed desired mass was detected. The residue was diluted with H₂O 10 mL and extracted with DCM 30 mL (10 mL×3). The combined organic layers were washed with brine 30 mL (10 mL×3), dried over Na₂SO₄, filtered and concentrated under reduced pressure to give a residue. The residue was purified by flash silica gel chromatography (Silica Flash Column, Eluent of 0-30% Ethyl acetate/Petroleum ether gradient @ 100 mL/min) to give 2,2-dimethyl-2,3-dihydroquinolin-4(1H)-one (2.3 g, 12.48 mmol, 42.06% yield, 95.1% purity) as a white solid.

To a solution of 2,2-dimethyl-2,3-dihydroquinolin-4(1H)-one (2.3 g, 13.1 mmol, 1.0 eq) in DCM (25 mL) was added pyridine (4.1 g, 52.5 mmol, 4.2 mL, 4.0 eq) at 0° C. And then TFAA (4.1 g, 19.6 mmol, 2.7 mL, 1.5 eq) was added and the mixture was stirred at 20° C. for 2 hr. LC-MS showed desired mass was detected. The reaction was stirred an additional 1.5 hr and poured into water (20 mL). The reaction was separated and the organic layer was washed with 1 M HCl (20 mL), brine (20 mL), dried over Na₂SO₄, and concentrated under reduced pressure. The residue was purified by flash silica gel chromatography (Silica Flash Column, Eluent of 0-10% Ethyl acetate/Petroleum ether gradient @ 100 mL/min) to give 2,2-dimethyl-1-(2,2,2-trifluoroacetyl)-2,3-dihydroquinolin-4(1 H)-one (2.7 g, 9.86 mmol, 75.16% yield, 99.1% purity) as a yellow solid.

¹H NMR (400 MHz, CHLOROFORM-d) δ 8.07 (dd, J=1.1, 7.8 Hz, 1H), 7.60-7.53 (m, 1H), 7.36 (t, J=7.6 Hz, 1H), 6.97 (d, J=8.1 Hz, 1H), 2.84 (s, 2H), 1.54 (s, 6H). To a solution of 2,2-dimethyl-1-(2,2,2-trifluoroacetyl)-2,3-dihydroquinolin-4(1H)-one (1.7 g, 6.2 mmol, 1.0 eq) in MeOH (20 mL) was added 5-(aminomethyl)-N-methyl-2-(methylthio)pyrimidin-4-amine (1.50 g, 8.15 mmol, 1.3 eq). Then AcOH (2.2 g, 37.6 mmol, 2.1 mL, 6.0 eq) was added to adjust pH to 5. The mixture was stirred at 70° C. for 2 hr. And then NaBH₃CN (1.1 g, 18.8 mmol, 3.0 eq) was added the mixture and stirred at 70° C. for 12 hr. LC-MS showed desired mass was detected. The reaction mixture was concentrated under reduced pressure to remove MeOH. The residue was diluted with H₂O 30 mL and extracted with EtOAc 100 mL. The combined organic layers were washed with brine (20 mL×3), dried over Na₂SO₄, filtered and concentrated under reduced pressure to give a residue. The residue was purified by flash silica gel chromatography (Silica Flash Column, Eluent of 0-50% Ethyl acetate/Petroleum ether gradient @ 100 mL/min) to give 1-(2,2-dimethyl-4-(((4-(methylamino)-2-(methylthio)pyrimidin-5-yl)methyl)amino)-3,4-dihydroquinolin-1(2H)-yl)-2,2,2-trifluoroethanone (330 mg, 750.86 umol, 11.98% yield) as yellow oil.

To a solution of 1-(2,2-dimethyl-4-(((4-(methylamino)-2-(methylthio)pyrimidin-5-yl)methyl)amino)-3,4-dihydroquinolin-1(2H)-yl)-2,2,2-trifluoroethanone (800.0 mg, 1.8 mmol, 1.0 eq) in DCM (10 mL) was added DIEA (705.7 mg, 5.4 mmol, 951.1 uL, 3.0 eq) and bis(trichloromethyl) carbonate (486.1 mg, 1.6 mmol, 0.9 eq) at 0° C. The mixture was stirred at 25° C. for 12 hr. LC-MS showed desired mass was detected. The residue was diluted with NaHCO₃ 15 mL and extracted with Dichloromethane (20 mL×3). The combined organic layers were washed with brine (15 mL×3), dried over Na₂SO₄, filtered and concentrated under reduced pressure to give a residue. The residue was purified by flash silica gel chromatography (Silica Flash Column, Eluent of 0-100% Ethyl acetate/Petroleum ether gradient @ 100 mL/min) to give 3-(2,2-dimethyl-1-(2,2,2-trifluoroacetyl)-1,2,3,4-tetrahydroquinolin-4-yl)-1-methyl-7-(methylthio)-3,4-dihydropyrimido[4,5-d]pyrimidin-2(1H)-one (520 mg, crude) as a yellow solid.

To a solution of 3-(2,2-dimethyl-1-(2,2,2-trifluoroacetyl)-1,2,3,4-tetrahydroquinolin-4-yl)-1-methyl-7-(methylthio)-3,4-dihydropyrimido[4,5-d]pyrimidin-2(1H)-one (520.0 mg, 1.1 mmol, 1.0 eq) in DCM (2 mL) was added m-CPBA (453.6 mg, 2.2 mmol, 85% purity, 2.0 eq) at 0° C. The mixture was stirred at 25° C. for 12 hr. LC-MS showed desired mass was detected. The residue was diluted with Na₂SO₃ 5 mL and extracted with DCM 30 mL (10 mL×3). The combined organic layers were washed with brine 30 mL (10 mL×3), dried over Na₂SO₄, filtered and concentrated under reduced pressure to give 3-(2,2-dimethyl-1-(2,2,2-trifluoroacetyl)-1,2,3,4-tetrahydroquinolin-4-yl)-1-methyl-7-(methylsulfonyl)-3,4-dihydropyrimido[4,5-d]pyrimidin-2(1H)-one (524 mg, crude) as a yellow solid.

To a solution of 3-(2,2-dimethyl-1-(2,2,2-trifluoroacetyl)-1,2,3,4-tetrahydroquinolin-4-yl)-1-methyl-7-(methylsulfonyl)-3,4-dihydropyrimido[4,5-d]pyrimidin-2(1H)-one (250.0 mg, 502.5 umol, 1.0 eq) in dioxane (3 mL) was added 4-(4-methylpiperazin-1-yl)aniline (96.1 mg, 502.5 umol, 1.0 eq) and TFA (85.9 mg, 753.7 umol, 55.8 uL, 1.5 eq). The mixture was stirred at 120° C. for 12 hr. LC-MS showed desired compound was detected. The reaction mixture was concentrated under reduced pressure to remove solvent. The residue was purified by prep-HPLC (FA condition; column: Phenomenex Luna C18 75*30 mm*3 um;mobile phase: [water(FA)-ACN];B %: 20%-50%, 8 min) to give 3-(2,2-dimethyl-1-(2,2,2-trifluoroacetyl)-1,2,3,4-tetrahydroquinolin-4-yl)-1-methyl-7-((4-(4-methylpiperazin-1-yl)phenyl)amino)-3,4-dihydropyrimido[4,5-d]pyrimidin-2(1H)-one (430 mg, 706.47 umol, 70.29% yield, 100% purity) as a brown solid.

A mixture of 3-(2,2-dimethyl-1-(2,2,2-trifluoroacetyl)-1,2,3,4-tetrahydroquinolin-4-yl)-1-methyl-7-((4-(4-methylpiperazin-1-yl)phenyl)amino)-3,4-dihydropyrimido[4,5-d]pyrimidin-2(1 H)-one (430 mg, 706.4 umol, 1.0 eq), NaOH (1 M, 2.5 mL) in MeOH (4 mL) was stirred at 20° C. for 8 hr. LC-MS showed desired mass was detected. The reaction mixture was concentrated. The reaction mixture was diluted with H₂O 10 mL and extracted with chloroform: Isopropyl alcohol=3:1 (10 mL×3). The combined organic layers were washed with brine (10 mL×3), dried over Na₂SO₄, filtered and concentrated under reduced pressure to give a residue. The residue was purified by prep-HPLC (HCl condition; column: Phenomenex Luna 80*30 mm*3 um;mobile phase: [water(HCl)-ACN];B %: 10%-35%, 8 min) to give 3-(2,2-dimethyl-1,2,3,4-tetrahydroquinolin-4-yl)-1-methyl-7-((4-(4-methylpiperazin-1-yl)phenyl)amino)-3,4-dihydropyrimido[4,5-d]pyrimidin-2(1H)-one (100 mg, 195.07 umol, 27.61% yield) as a yellow solid.

To a solution of 3-(2,2-dimethyl-1,2,3,4-tetrahydroquinolin-4-yl)-1-methyl-7-((4-(4-methylpiperazin-1-yl)phenyl)amino)-3,4-dihydropyrimido[4,5-d]pyrimidin-2(1H)-one (25.0 mg, 45.5 umol, 1.0 eq, HCl) in DMF (2 mL) was added TEA (23.04 mg, 227.64 umol, 31.69 uL, 5 eq) and prop-2-enoyl chloride (4.1 mg, 45.5 umol, 3.71 uL, 1.0 eq) in DMF (0.1 mL) at 0° C. The mixture was stirred at 0° C. for 1 hr. LC-MS showed desired compound was detected. The reaction mixture was filtered and to give a residue. The residue was purified by prep-HPLC (FA condition, column: Phenomenex Luna C18 75*30 mm*3 um;mobile phase: [water(FA)-ACN];B %: 1%-30%, 8 min) to give 3-(1-acryloyl-2,2-dimethyl-1,2,3,4-tetrahydroquinolin-4-yl)-1-methyl-7-((4-(4-methylpiperazin-1-yl)phenyl)amino)-3,4-dihydropyrimido[4,5-d]pyrimidin-2(1H)-one (8 mg, 8.75 umol, 19.22% yield, 62% purity) as a yellow solid. MS (ESI): m/z=567.2 [M+H]⁺

### Procedure for Preparation of Compound 108

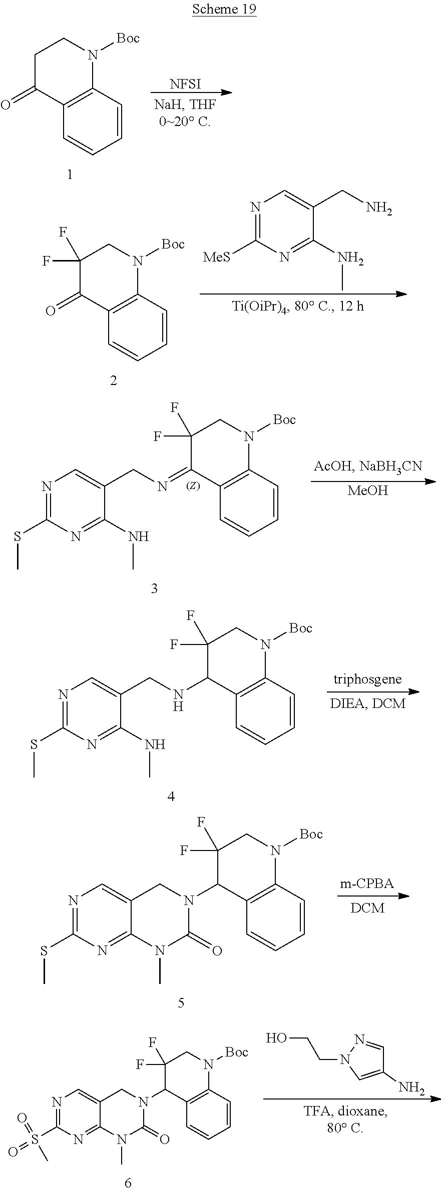

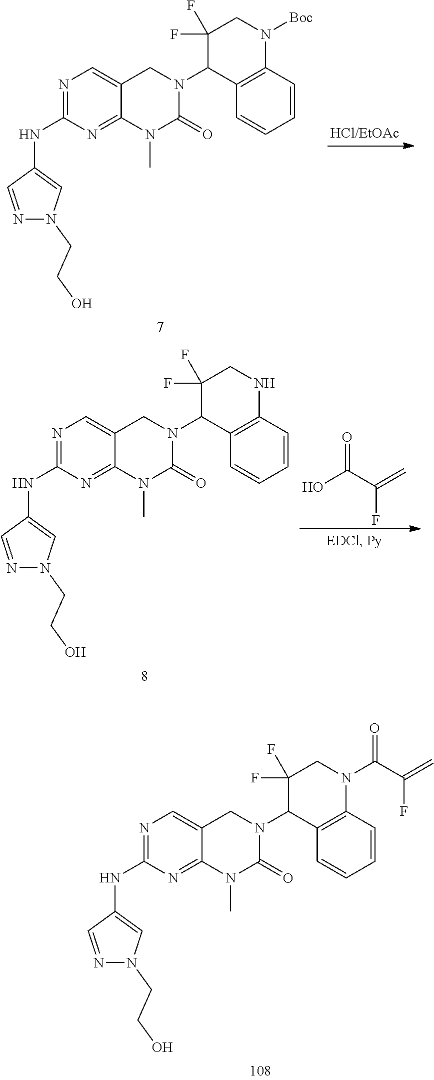

To a solution of tert-butyl 4-oxo-2,3-dihydroquinoline-1-carboxylate (1 g, 4.04 mmol, 1 eq) in THF(10 mL) was added NaH (970.52 mg, 24.26 mmol, 60% purity, 6 eq) at 0° C. The mixture was stirred at 20° C. for 3 hr under N₂ atmosphere. A solution of N-(benzenesulfonyl)-N-fluoro-benzenesulfonamide (5.10 g, 16.18 mmol, 4 eq) in THE (10 mL) was added in the mixture at 20° C. The mixture was stirred at 20° C. for 40 hr under N₂ atmosphere. TLC indicated reactant 1 was remained and two new spots formed. The reaction mixture was quenched with NH₄Cl solution (60 mL) at 0° C., then the mixture was extracted with EtOAc (80 mL*3). The combined organic phase was dried with anhydrous Na₂SO₄, the mixture was filtered and the filtrate was concentrated under vacuum. The residue was purified by flash silica gel chromatography (ISCO®; 20 g SepaFlash® Silica Flash Column, Eluent of 0-10% Ethyl acetate/Petroleum ether gradient @ 30 mL/min) to give tert-butyl 3,3-difluoro-4-oxo-2H-quinoline-1-carboxylate (1.15 g) as a yellow solid.

1H NMR (400 MHz, CHLOROFORM-d) δ ppm 8.07 (dd, J=7.88, 1.63 Hz, 1H), 7.81 (d, J=8.38 Hz, 1H), 7.62 (ddd, J=8.57, 7.13, 1.69 Hz, 1H), 7.28-7.36 (m, 1H), 4.42 (t, J=12.26 Hz, 2H), 1.58 (s, 9H)

To a solution of tert-butyl 3,3-difluoro-4-oxo-2H-quinoline-1-carboxylate (650 mg, 2.29 mmol, 1 eq) and 5-(aminomethyl)-N-methyl-2-methylsulfanyl-pyrimidin-4-amine (845.62 mg, 4.59 mmol, 2 eq) in Tol. (20 mL) was added Ti(i-PrO)₄ (1.96 g, 6.88 mmol, 2.03 mL, 3 eq) at 20° C. The mixture was stirred at 80° C. for 12 hr. TLC indicated reactant was consumed completely and three new spots formed. The reaction mixture was quenched with 20 mL NH₄Cl solution. The mixture was filtered and the filtrate was concentrated under vacuum. The residue was diluted with 20 mL H₂O and extracted with EtOAc (20 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-8% Ethyl acetate/Petroleum ether gradient @ 15 mL/min) to give tert-butyl (4Z)—S-fluoro-4-[[4-(methylamino)-2-methylsulfanyl-pyrimidin-5-yl]methylimino]quinoline-1-carboxylate (340 mg, 791.60 umol, 34.50% yield) as a yellow oil and tert-butyl (4Z)-3,3-difluoro-4-[[4-(methylamino)-2-methylsulfanyl-pyrimidin-5-yl]methylimino]-2H-quinoline-1-carboxylate (620 mg, 1.38 mmol, 60.11% yield) as a yellow solid.

To a solution of tert-butyl (4Z)-3,3-difluoro-4-[[4-(methylamino)-2-methylsulfanyl-pyrimidin-5-yl]methylimino]-2H-quinoline-1-carboxylate (620 mg, 1.38 mmol, 1 eq) in MeOH (15 mL) was added AcOH (91.11 mg, 1.52 mmol, 86.77 uL, 1.1 eq) and NaBH₃CN (95.34 mg, 1.52 mmol, 1.1 eq) at 0° C. The mixture was stirred at 20° C. for 24 hr. LCMS detected the formation of the desired mass peak. The mixture was concentrated under vacuum. The residue was diluted with 30 mL brine and extracted with EtOAc (10 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-10% Ethyl acetate/Petroleum ether gradient @ 15 mL/min) to give tert-butyl 3,3-difluoro-4-[[4-(methylamino)-2-methylsulfanyl-pyrimidin-5-yl]methylamino]-2,4-dihydroquinoline-1-carboxylate (380 mg, 841.58 umol, 61.02% yield) as a yellow oil.

To a solution of tert-butyl 3,3-difluoro-4-[[4-(methylamino)-2-methylsulfanyl-pyrimidin-5-yl]methylamino]-2,4-dihydroquinoline-1-carboxylate (570 mg, 1.26 mmol, 1 eq) in DCM(8 mL) was added DIPEA (489.46 mg, 3.79 mmol, 659.65 uL, 3 eq) at 0° C. under N₂ atmosphere. Then bis(trichloromethyl) carbonate (930 mg, 3.13 mmol, 2.48 eq) in DCM (2 mL) was added in the mixture at 0° C. under N₂ atmosphere. The mixture was stirred at 20° C. for 3 hr under N₂ atmosphere. LCMS detected the formation of the desired mass peak. The reaction mixture was quenched with NaHCO₃ solution (20 mL) at 0° C., then the mixture was extracted with DCM (10 mL*3). The combined organic phases were dried with anhydrous Na₂SO₄, filtered and concentrated under vacuum. The residue was purified by flash silica gel chromatography (ISCO®; 4 g SepaFlash® Silica Flash Column, Eluent of 0-30% Ethyl acetate/Petroleum ether gradient @ 10 mL/min) to give tert-butyl 3,3-difluoro-4-(1-methyl-7-methylsulfanyl-2-oxo-4H-pyrimido[4,5-d]pyrimidin-S-yl)-2,4-dihydroquinoline-1-carboxylate (540 mg, 1.13 mmol, 89.58% yield) as a pale yellow solid.

To a solution of tert-butyl 3,3-difluoro-4-(1-methyl-7-methylsulfanyl-2-oxo-4H-pyrimido[4,5-d]pyrimidin-S-yl)-2,4-dihydroquinoline-1-carboxylate (540 mg, 1.13 mmol, 1 eq) in DCM (20 mL) was added MCPBA (459.17 mg, 2.26 mmol, 85% purity, 2 eq) at 0° C. The mixture was stirred at 20° C. for 12 hr. LCMS detected the formation of the desired mass peak. The reaction mixture was quenched with saturated Na₂SO₃ aqueous solution(10 mL) at 0° C., the reaction mixture was diluted with 45 mL brine and extracted with EtOAc (15 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-32% Ethyl acetate/Petroleum ether gradient @ 20 mL/min) to give tert-butyl 3,3-difluoro-4-(1-methyl-7-methylsulfonyl-2-oxo-4H-pyrimido[4,5-d]pyrimidin-S-yl)-2,4-dihydroquinoline-1-carboxylate (550 mg, 1.08 mmol, 95.46% yield) as a pale yellow solid.

To a solution of tert-butyl 3,3-difluoro-4-(1-methyl-7-methylsulfonyl-2-oxo-4H-pyrimido[4,5-d]pyrimidin-S-yl)-2,4-dihydroquinoline-1-carboxylate (550 mg, 1.08 mmol, 1 eq) and 2-(4-aminopyrazol-1-yl)ethanol (274.49 mg, 2.16 mmol, 2 eq) in dioxane (20 mL) was added TFA (184.62 mg, 1.62 mmol, 119.88 uL, 1.5 eq) at 20° C. The mixture was stirred at 80° C. for 12 hr under N₂ atmosphere. LCMS detected the formation of the desired mass peak. The mixture was concentrated under vacuum. The residue was diluted with 20 mL brine and extracted with EtOAc (10 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-5% MeOH/DCM @ 15 mL/min) to give tert-butyl 3,3-difluoro-4-[7-[[1-(2-hydroxyethyl)pyrazol-4-yl]amino]-1-methyl-2-oxo-4H-pyrimido[4,5-d]pyrimidin-S-yl]-2,4-dihydroquinoline-1-carboxylate (530 mg, 923.70 umol, 85.57% yield, 97% purity) as a yellow solid.

1H NMR (400 MHz, CHLOROFORM-d) δ ppm 7.70-7.86 (m, 3H), 7.56 (br s, 1H), 7.36 (s, 1H), 7.01-7.22 (m, 2H), 6.16 (br dd, J=19.70, 12.32 Hz, 1H), 4.58-4.71 (m, 1H), 4.21-4.27 (m, 2H), 4.15 (br d, J=14.63 Hz, 1H), 3.94-4.04 (m, 3H), 3.62-3.76 (m, 1H), 3.56 (s, 3H), 1.57 (s, 9H)

A solution of tert-butyl 3,3-difluoro-4-[7-[[1-(2-hydroxyethyl)pyrazol-4-yl]amino]-1-methyl-2-oxo-4H-pyrimido[4,5-d]pyrimidin-S-yl]-2,4-dihydroquinoline-1-carboxylate (300 mg, 539.02 umol, 1 eq) in HCl/EtOAc (4 M, 6 mL) at 20° C. for 1 hr. LCMS showed the reaction was completed and desired product mass peak was detected. The reaction mixture was concentrated under reduced pressure to to give 3-(3,3-difluoro-2,4-dihydro-1H-quinolin-4-yl)-7-[[1-(2-hydroxyethyl)pyrazol-4-yl]amino]-1-methyl-4H-pyrimido[4,5-d]pyrimidin-2-one (265 mg, crude, HCl) as a pale yellow solid.

To a solution of 3-(3,3-difluoro-2,4-dihydro-1 H-quinolin-4-yl)-7-[[1-(2-hydroxyethyl)pyrazol-4-yl]amino]-1-methyl-4H-pyrimido[4,5-d]pyrimidin-2-one (265 mg, 537.62 umol, 1 eq, HCl) and 2-fluoroprop-2-enoic acid (145.24 mg, 1.61 mmol, 3 eq) in Pyridine (4 mL) was added EDCI (206.13 mg, 1.08 mmol, 2 eq) at 20° C. The mixture was stirred at 50° C. for 12 hr. LCMS showed the reaction was completed and desired product was detected. The mixture was concentrated under vacuum. The residue was dissolved in DMF (3 mL), filtered through a syringe filter, and then purified by prep-HPLC (neutral condition; column: Waters Xbridge Prep OBD C18 150*40 mm*10 um;mobile phase: [water(NH4HCO3)-ACN];B %: 35%-65%, 8 min) to give 3-[3,3-difluoro-1-(2-fluoroprop-2-enoyl)-2,4-dihydroquinolin-4-yl]-7-[[1-(2-hydroxyethyl)pyrazol-4-yl]amino]-1-methyl-4H-pyrimido[4,5-d]pyrimidin-2-one (60.2 mg, 104.74 umol, 19.48% yield, 91.95% purity) as a pale yellow solid. MS (ESI): m/z=529.3 [M+H]⁺

### Procedure for Preparation of Compound 110

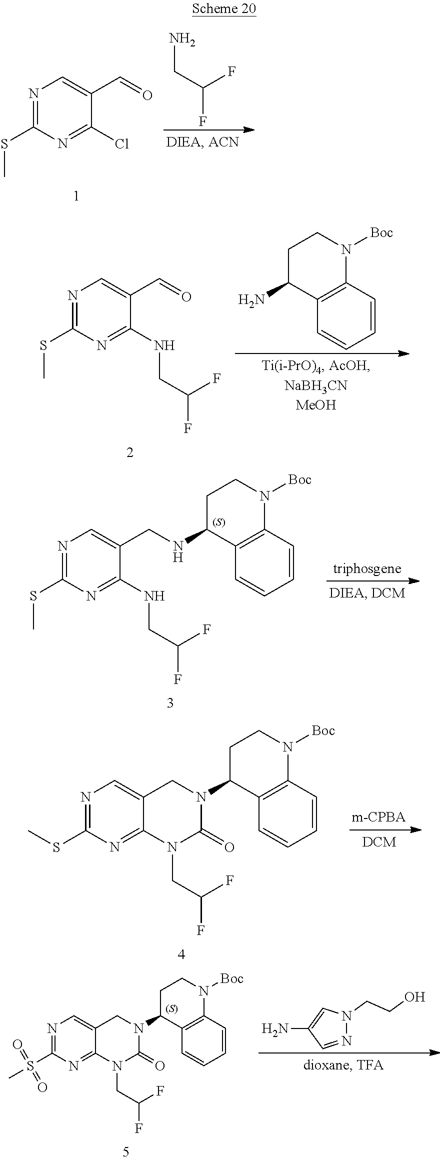

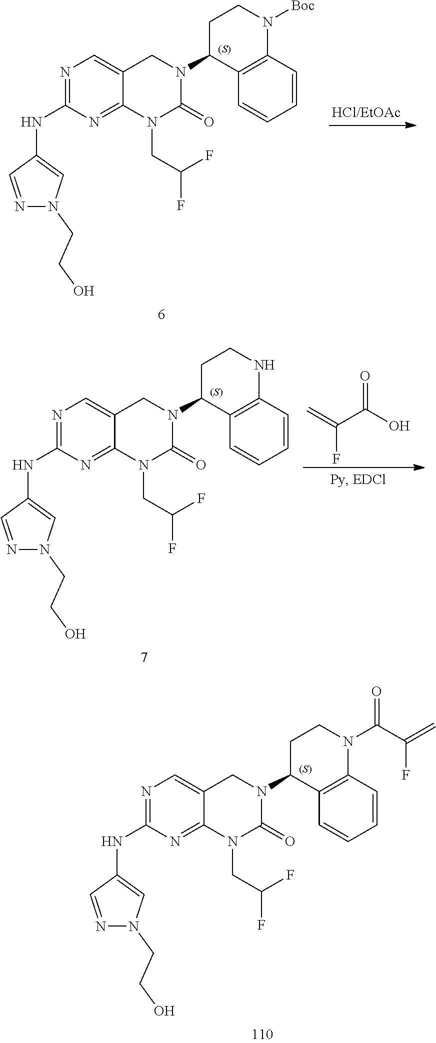

To a solution of 4-chloro-2-(methylthio)pyrimidine-5-carbaldehyde (1.0 g, 5.3 mmol, 1.0 eq) in ACN (10 mL) was added DIEA (2.0 g, 15.9 mmol, 2.7 mL, 3.0 eq) and 2,2-difluoroethanamine hydrochloride (429.7 mg, 5.3 mmol, 1.0 eq). The mixture was stirred at 50° C. for 12 hr. LC-MS showed desired compound was detected. The reaction mixture was concentrated under reduced pressure to remove solvent. The residue was purified by flash silica gel chromatography (Silica Flash Column, Eluent of 0-50% Ethyl acetate/Petroleum ether gradient @ 120 mL/min) to give 4-((2,2-difluoroethyl)amino)-2-(methylthio)pyrimidine-5-carbaldehyde (1.0 g, 2.4 mmol, 46.5% yield, 56.4% purity) as a yellow solid.

To a solution of 4-((2,2-difluoroethyl)amino)-2-(methylthio)pyrimidine-5-carbaldehyde (1.0 g, 4.3 mmol, 1.0 eq) in MeOH (20 mL) was added (S)-tert-butyl 4-amino-3,4-dihydroquinoline-1(2H)-carboxylate (1.0 g, 4.3 mmol, 1.0 eq), Ti(i-PrO)₄ (3.7 g, 13.1 mmol, 3.8 mL, 3.0 eq) and AcOH (1.3 g, 21.8 mmol, 1.2 mL, 5.0 eq), stirred at 70° C. for 12 hr. NaBH₃CN (1.6 g, 26.2 mmol, 6.0 eq) was added. The mixture was stirred at 70° C. for 2 hr. LC-MS showed desired compound was detected. The reaction mixture was concentrated under reduced pressure to remove solvent. The reaction mixture was diluted with NH₄Cl 50 mL and extracted with EtOAc 150 mL (50 mL×3). The combined organic layers were washed with brine 100 mL (50 mL×2), dried over Na₂SO₄, filtered and the filtrate was concentrated under reduced pressure to give a residue. The residue was purified by flash silica gel chromatography (Silica Flash Column, Eluent of 0-60% Ethyl acetate/Petroleum ether gradient @ 120 mL/min) to give (S)-tert-butyl 4-(((4-((2,2-difluoroethyl)amino)-2-(methylthio)pyrimidin-5-yl)methyl)amino)-3,4-dihydroquinoline-1(2H)-carboxylate (1.0 g, 1.0 mmol, 24.8% yield, 47.3% purity) as yellow oil.

To a solution of (S)-tert-butyl 4-(((4-((2,2-difluoroethyl)amino)-2-(methylthio)pyrimidin-5-yl)methyl)amino)-3,4-dihydroquinoline-1(2H)-carboxylate (1.0 g, 2.3 mmol, 1.0 eq) in DCM (20 mL) was added DIEA (891.1 mg, 6.8 mmol, 1.2 mL, 3.0 eq) and bis(trichloromethyl) carbonate (682.0 mg, 2.3 mmol, 1.0 eq) at 0° C., then stirred at 20° C. for 2 hr. LC-MS showed desired compound was detected. The reaction mixture was partitioned between NaHCO₃ 100 mL and DCM 100 mL. The water phase was separated, extracted with DCM 300 mL (100 mL×3), dried over Na₂SO₄, filtered and concentrated under reduced pressure to give a residue. The residue was purified by flash silica gel chromatography (Silica Flash Column, Eluent of 0-50% Ethyl acetate/Petroleum ether gradient @ 120 mL/min) to give (S)-tert-butyl 4-(1-(2,2-difluoroethyl)-7-(methylthio)-2-oxo-1,2-dihydropyrimido[4,5-d]pyrimidin-S(4H)-yl)-3,4-dihydroquinoline-1(2H)-carboxylate (540.0 mg, 953.1 umol, 41.4% yield, 86.7% purity) as colorless oil.

To a solution of (S)-tert-butyl 4-(1-(2,2-difluoroethyl)-7-(methylthio)-2-oxo-1,2-dihydropyrimido[4,5-d]pyrimidin-S(4H)-yl)-3,4-dihydroquinoline-1(2H)-carboxylate (540.0 mg, 1.1 mmol, 1.0 eq) in DCM (10 mL) was added m-CPBA (334.5 mg, 1.6 mmol, 85% purity, 1.5 eq) at 0° C. The mixture was stirred at 25° C. for 1 hr. LC-MS showed desired compound was detected. The reaction mixture was diluted with Na₂SO₃ 20 mL and extracted with DCM 45 mL (15 mL×3). The combined organic layers were washed with NaHCO₃ 45 mL (15 mL×2), dried over Na₂SO₄, filtered and the filtrate was concentrated under reduced pressure to give (S)-tert-butyl 4-(1-(2,2-difluoroethyl)-7-(methylsulfonyl)-2-oxo-1,2-dihydropyrimido[4,5-d]pyrimidin-S(4H)-yl)-3,4-dihydroquinoline-1(2H)-carboxylate (580 mg, crude) as a white solid.

To a solution of (S)-tert-butyl 4-(1-(2,2-difluoroethyl)-7-(methylsulfonyl)-2-oxo-1,2-dihydropyrimido[4,5-d]pyrimidin-S(4H)-yl)-3,4-dihydroquinoline-1(2H)-carboxylate (50.0 mg, 95.5 umol, 1.0 eq) in dioxane (2 mL) was added TFA (16.3 mg, 143.2 umol, 10.6 uL, 1.5 eq) and 2-(4-amino-1H-pyrazol-1-yl)ethanol (14.5 mg, 114.6 umol, 1.2 eq). The mixture was stirred at 80° C. for 12 hr. LC-MS showed Reactant 5 remained. Another batch of 2-(4-aminopyrazol-1-yl)ethanol (18.2 mg, 143.2 umol, 1.5 eq) and TFA (16.3 mg, 143.2 umol, 10.6 uL, 1.5 eq) was added to the reaction and stirred at 100° C. for extra 12 hr. LC-MS showed desired compound was detected. The reaction mixture was concentrated under reduced pressure to remove solvent. The residue was purified by prep-HPLC (TFA condition; column: Phenomenex Luna 80*30 mm*3 um;mobile phase: [water(FA)-ACN];B %: 65%-90%, 8 min) to give (R)-tert-butyl 4-(1-(2,2-difluoroethyl)-7-((1-(2-hydroxyethyl)-1 H-pyrazol-4-yl)amino)-2-oxo-1,2-dihydropyrimido[4,5-d]pyrimidin-S(4H)-yl)-3,4-dihydroquinoline-1(2H)-carboxylate (44.0 mg, crude) as a yellow oil.

A mixture of (R)-tert-butyl 4-(1-(2,2-difluoroethyl)-7-((1-(2-hydroxyethyl)-1 H-pyrazol-4-yl)amino)-2-oxo-1,2-dihydropyrimido[4,5-d]pyrimidin-S(4H)-yl)-3,4-dihydroquinoline-1(2H)-carboxylate (44.0 mg, 77.1 umol, 1.0 eq) in HCl/EtOAc (2 mL, 4 M) was stirred at 20° C. for 30 min. LC-MS showed desired compound was detected. The reaction mixture was concentrated under reduced pressure to give (R)-1-(2,2-difluoroethyl)-7-((1-(2-hydroxyethyl)-1H-pyrazol-4-yl)amino)-S-(1,2,3,4-tetrahydroquinolin-4-yl)-3,4-dihydropyrimido[4,5-d]pyrimidin-2(1H)-one (50.0 mg, crude) as a white solid.

To a solution of (R)-1-(2,2-difluoroethyl)-7-((1-(2-hydroxyethyl)-1H-pyrazol-4-yl)amino)-S-(1,2,3,4-tetrahydroquinolin-4-yl)-3,4-dihydropyrimido[4,5-d]pyrimidin-2(1 H)-one (50.0 mg, 106.2 umol, 1.0 eq) in pyridine (2 mL) was added EDCI (61.1 mg, 318.8 umol, 3.0 eq) and 2-fluoroacrylic acid (19.1 mg, 212.5 umol, 2.0 eq). The mixture was stirred at 20° C. for 12 hr. LC-MS showed desired compound was detected. The reaction mixture was concentrated under reduced pressure. The residue was purified by prep-HPLC (FA condition; column: Phenomenex Luna C18 75*30 mm*3 um;mobile phase: [water(FA)-ACN];B %: 1%-50%, 8 min) to give 1-(2,2-difluoroethyl)-S-[(4S)-1-(2-fluoroprop-2-enoyl)-3,4-dihydro-2H-quinolin-4-yl]-7-[[1-(2-hydroxyethyl)pyrazol-4-yl]amino]-4H-pyrimido[4,5-d]pyrimidin-2-one (8.1 mg, 14.7 umol, 13.9% yield, 98.9% purity) as a white solid.

MS (ESI): m/z=543.2 [M+H]⁺

### Procedure for Preparation of Compound 112

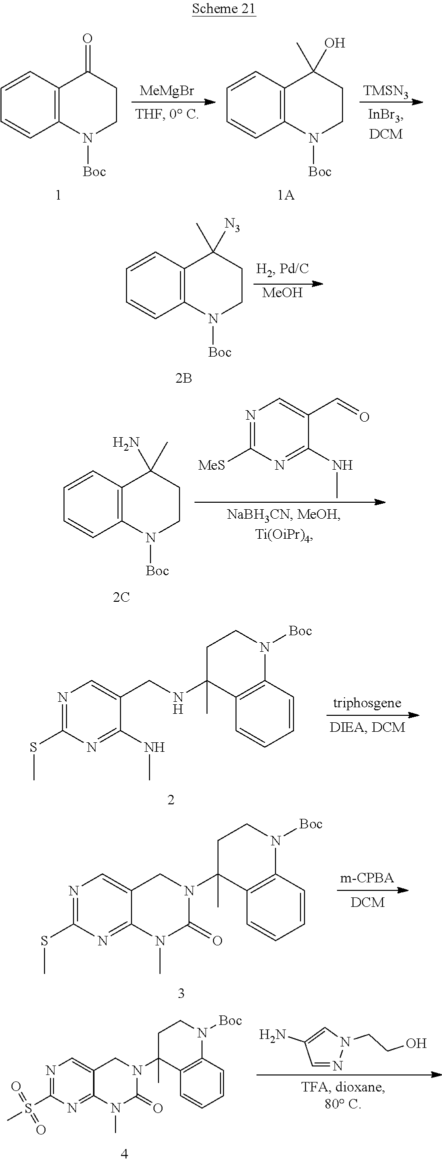

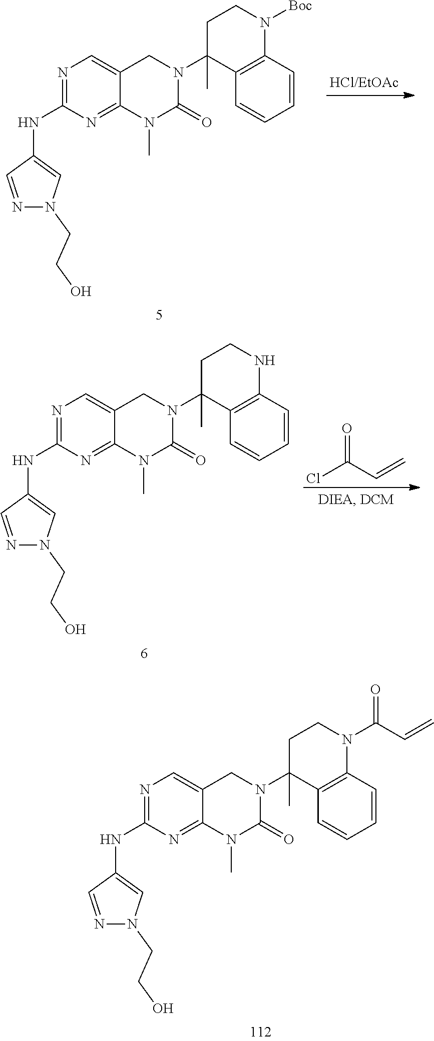

A mixture of tert-butyl 4-oxo-2,3-dihydroquinoline-1-carboxylate (3 g, 12.1 mmol, 1 eq) in THE (100 mL) which was cooled to 0° C. was degassed and purged with N₂ for 3 times, then MeMgBr (3 M, 20.2 mL, 5 eq) was added and then the mixture was stirred at 0° C. for 2 h under N₂ atmosphere. LC-MS showed desired compound was detected. The mixture was quenched by Sat. NH₄Cl 100 mL and then extracted by ethyl acetate (30 mL×3), the combined organic layers was dried by Na₂SO₄, filtered and the filtrate was concentrated to get a residue. The residue was purified by column chromatography (SiO₂, Petroleum ether/Ethyl acetate=50/1 to 5/1) to give tert-butyl 4-hydroxy-4-methyl-2, 3-dihydroquinoline-1-carboxylate (1.8 g, 6.8 mmol, 56.3% yield) as a yellow oil.

TMSN₃ (1.7 g, 14.8 mmol, 1.9 mL, 3 eq) was added to a mixture of tert-butyl 4-hydroxy-4-methyl-2,3-dihydroquinoline-1-carboxylate (1.3 g, 4.9 mmol, 1 eq) and tribromoindigane (175.0 mg, 493.7 umol, 0.1 eq) in DCM (100 mL), the mixture was stirred at 0° C. for 1 h. TLC showed desired compound was detected. The mixture was filtered and the filtrate was concentrated to get a residue. The residue was purified by column chromatography (SiO₂, Petroleum ether/Ethyl acetate=50/1 to 30/1) to give tert-butyl 4-azido-4-methyl-2, 3-dihydroquinoline-1-carboxylate (1.3 g, 4.5 mmol, 91.3% yield) as a colorless oil.

Pd/C (300 mg, 10% purity) was added to a mixture of tert-butyl 4-azido-4-methyl-2, 3-dihydroquinoline-1-carboxylate (1.3 g, 4.5 mmol, 1 eq) in MeOH (10 mL), the mixture was evacuated and recharged with H₂ for 3 times and then stirred under H₂ (15 psi) at 20° C. for 1 h. LC-MS showed desired compound was detected. The mixture was filtered through a celite plug and the filtrate was concentrated to get compound tert-butyl 4-amino-4-methyl-2, 3-dihydroquinoline-1-carboxylate (1.1 g, crude) as a colorless oil.

To a solution of tert-butyl 4-amino-4-methyl-2,3-dihydroquinoline-1-carboxylate (1 g, 3.8 mmol, 1 eq) 4-(methylamino)-2-methylsulfanyl-pyrimidine-5-carbaldehyde (698.4 mg, 3.8 mmol, 1 eq) in MeOH (20 mL) was added Ti(i-PrO)₄ (3.3 g, 11.4 mmol, 3.4 mL, 3 eq), the mixture pH was adjusted to 4-5 by AcOH and stirred at 65° C. for 12 h, and then NaBH₃CN (718.6 mg, 11.4 mmol, 3 eq) was added. The mixture was stirred at 65° C. for 2 h. LC-MS showed desired compound was detected. The reaction mixture was quenched by Sat·NH₄Cl 20 mL, then extracted with ethyl acetate (20 mL×3). The combined organic layers were dried by Na₂SO₄, filtered and the filtrate was concentrated to get a residue. The residue was purified by column chromatography (SiO₂, Petroleum ether/Ethyl acetate=10/1 to 1/1) to afford tert-butyl 4-methyl-4-[[4-(methylamino)-2-methylsulfanyl-pyrimidin-5-yl] methylamino]-2, 3-dihydroquinoline-1-carboxylate (1.3 g, 3.0 mmol, 79.4% yield) as a colorless oil.

To a solution of tert-butyl 4-methyl-4-[[4-(methylamino)-2-methylsulfanyl-pyrimidin-5-yl]methylamino]-2,3-dihydroquinoline-1-carboxylate (1.3 g, 3.0 mmol, 1 eq) and DIEA (1.2 g, 9.1 mmol, 1.6 mL, 3 eq) in DCM (10 mL) was added bis(trichloromethyl) carbonate (898.0 mg, 3.0 mmol, 1 eq) in DCM (5 mL) at 0° C. Then the mixture was stirred at 20° C. for 12 h.

LC-MS showed desired compound was detected. The mixture was quenched by Sat·NaHCO₃ 50 mL and then extracted with DCM (30 mL×3). The combined organic layers were washed with brine (10 mL×2), dried over Na₂SO₄, filtered and concentrated under reduced pressure to give a residue. The residue was purified by column chromatography (SiO₂, Petroleum ether/Ethyl acetate=10/1 to 1/1) to give tert-butyl 4-methyl-4-(1-methyl-7-methylsulfanyl-2-oxo-4H-pyrimido [4, 5-d]pyrimidin-S-yl)-2,3-dihydroquinoline-1-carboxylate (1.2 g, 2.6 mmol, 87.0% yield) as a colorless oil.

To a solution of tert-butyl 4-methyl-4-(1-methyl-7-methylsulfanyl-2-oxo-4H-pyrimido[4,5-d]pyrimidin-S-yl)-2,3-dihydroquinoline-1-carboxylate (1.1 g, 2.4 mmol, 1 eq) in DCM (20 mL) was added m-CPBA (931.4 mg, 4.6 mmol, 85% purity, 1.9 eq) at 0° C. Then the mixture was stirred at 20° C. for 1 h. LC-MS showed desired compound was detected. The reaction mixture was concentrated under reduced pressure to remove solvent. The reaction mixture was partitioned between Na₂SO₃ 20 mL and dichloromethane (20×3 mL). The organic phase was separated, washed with NaHCO₃ (50 mL×2), dried over Na₂SO₄, filtered and concentrated under reduced pressure to give tert-butyl 4-methyl-4-(1-methyl-7-methylsulfonyl-2-oxo-4H-pyrimido[4,5-d]pyrimidin-S-yl)-2,3-dihydroquinoline-1-carboxylate (1.2 g, crude) as a colorless oil.

To a solution of tert-butyl 4-methyl-4-(1-methyl-7-methylsulfonyl-2-oxo-4H-pyrimido[4,5-d]pyrimidin-S-yl)-2,3-dihydroquinoline-1-carboxylate (200 mg, 410.2 umol, 1 eq) in dioxane (3 mL) was added TFA (56.1 mg, 492.2 umol, 36.4 uL, 1.2 eq) and 2-(4-aminopyrazol-1-yl)ethanol (52.2 mg, 410.2 umol, 1 eq). The mixture was stirred at 80° C. for 12 h. LC-MS showed desired compound was detected. The mixture was poured into Sat·NaHCO₃ 20 mL and then extracted by ethyl acetate (10 mL×3), then dried by Na₂SO₄, filtered and the filtrate was concentrated to get a residue. The residue was purified by column chromatography (SiO₂, Petroleum ether/Ethyl acetate=3/1 to 0/1) to give compound tert-butyl 4-[7-[[1-(2-hydroxyethyl)pyrazol-4-yl]amino]-1-methyl-2-oxo-4H-pyrimido[4,5-d]pyrimidin-S-yl]-4-methyl-2,3-dihydroquinoline-1-carboxylate (220 mg, 411.5 umol, 33.4% yield) as a yellow oil.

To a solution of tert-butyl 4-[7-[[1-(2-hydroxyethyl) pyrazol-4-yl]amino]-1-methyl-2-oxo-4H-pyrimido[4,5-d]pyrimidin-S-yl]-4-methyl-2,3-dihydroquinoline-1-carboxylate (220 mg, 411.5 umol, 1 eq) in EtOAc (5 mL) was added HCl/EtOAc (5 mL, 4 M). The mixture was stirred at 20° C. for 1 h. LC-MS showed desired compound was detected. The reaction was concentrated to give 7-[[1-(2-hydroxyethyl) pyrazol-4-yl] amino]-1-methyl-S-(4-methyl-2, 3-dihydro-1H-quinolin-4-yl)-4H-pyrimido [4, 5-d] pyrimidin-2-one (220 mg, crude, HCl) as a yellow solid.

To a solution of 7-[[1-(2-hydroxyethyl)pyrazol-4-yl]amino]-1-methyl-S-(4-methyl-2,3-dihydro-1 H-quinolin-4-yl)-4H-pyrimido[4,5-d]pyrimidin-2-one (90 mg, 191.1 umol, 1 eq, HCl) in DCM (2 mL) was added DIEA (123.5 mg, 955.5 umol, 166.4 uL, 5 eq). The mixture was cooled to 0° C., then a solution of prop-2-enoyl chloride (10.4 mg, 114.7 umol, 9.4 uL, 0.6 eq) in DCM (0.15 mL) was added, the mixture was stirred at 0° C. for 1 h. LC-MS showed desired compound was detected. The solvent was removed by a stream of N₂ to get a residue. The residue was purified by prep-HPLC (FA condition; column: Phenomenex Luna C18 200×40 mm×10 um; mobile phase: [water(FA)-ACN];B %: 1%-40%, 8 min) to give 7-[[1-(2-hydroxyethyl)pyrazol-4-yl]amino]-1-methyl-S-(4-methyl-1-prop-2-enoyl-2,3-dihydroquinolin-4-yl)-4H-pyrimido[4,5-d]pyrimidin-2-one (11 mg, 20.6 umol, 10.8% yield, 93.26% purity, 0.2 FA) as a gray solid. MS (ESI): m/z=489.2 [M+H]⁺

### Procedure for Preparation of Compound 113

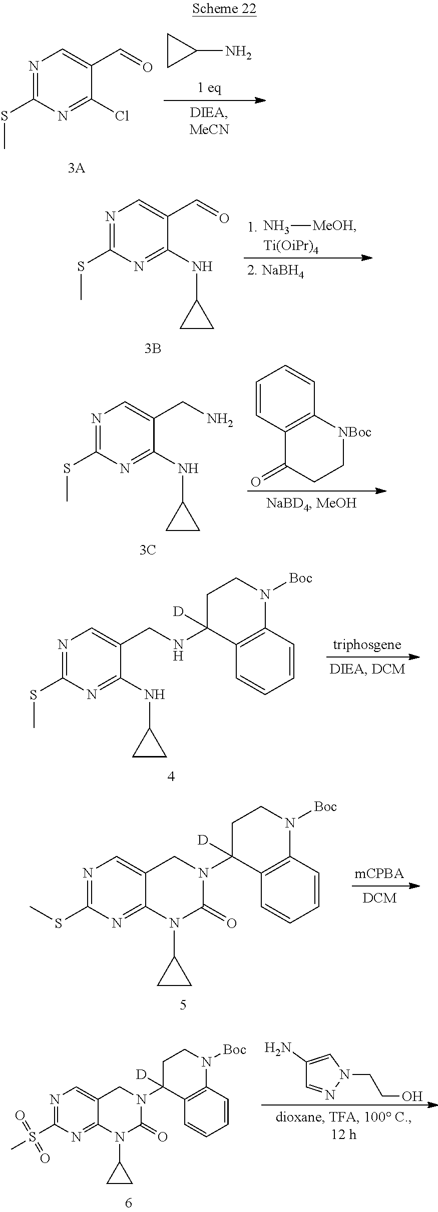

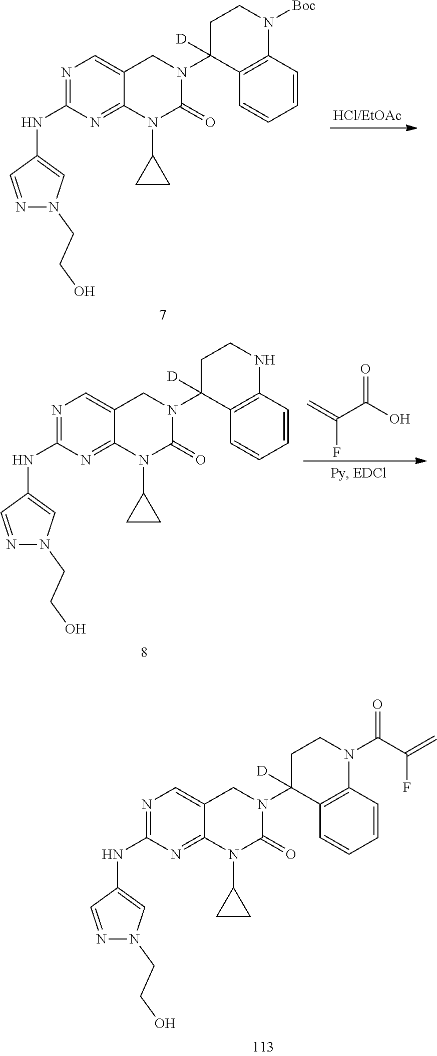

To a solution of 4-chloro-2-methylsulfanyl-pyrimidine-5-carbaldehyde (1 g, 5.3 mmol, 1 eq) in MeCN (10 mL) was added DIEA (2.1 g, 15.9 mmol, 2.8 mL, 3 eq) and cyclopropanamine (302.7 mg, 5.3 mmol, 367.3 uL, 1 eq). The mixture was stirred at 50° C. for 2 hr. LC-MS showed desired compound was detected. The reaction mixture was concentrated under reduced pressure to remove solvent to give 4-(cyclopropylamino)-2-methylsulfanyl-pyrimidine-5-carbaldehyde (1.1 g, crude) as yellow oil.

A mixture of 4-(cyclopropylamino)-2-methylsulfanyl-pyrimidine-5-carbaldehyde (2 g, 9.6 mmol, 1 eq) and tetraisopropoxytitanium (8.2 g, 28.7 mmol, 8.5 mL, 3 eq) in NH₃/MeOH (10 M, 100 mL, 104.6 eq) was stirred at 20° C. for 12 hr. NaBH₄ (1.8 g, 47.8 mmol, 5 eq) was then added to the reaction at 0° C., and stirred at 20° C. for 2 hr. LC-MS showed desired compound was detected. The reaction mixture was concentrated under reduced pressure to remove solvent. The reaction mixture was diluted with Sat·NH₄Cl 50 mL and extracted with ethyl acetate 150 mL (50 mL×3). The combined organic layers were washed with brine 100 mL (50 mL×2), dried over Na₂SO₄, filtered and the filtrate was concentrated under reduced pressure. The residue was purified by flash silica gel chromatography (40 g Silica Flash Column, Eluent of 0-30% Ethyl acetate/Petroleum ether gradient @ 70 mL/min) to give 5-(aminomethyl)-N-cyclopropyl-2-methylsulfanyl-pyrimidin-4-amine (780 mg, 3.7 mmol, 38.3% yield, 98.8% purity) as yellow oil.

To a solution of 5-(aminomethyl)-N-cyclopropyl-2-methylsulfanyl-pyrimidin-4-amine (530 mg, 2.5 mmol, 1 eq) in MeOH (10 mL) was added Ti(i-PrO)₄ (2.2 g, 7.6 mmol, 2.2 mL, 3 eq) and tert-butyl 4-oxo-2,3-dihydroquinoline-1-carboxylate (623.2 mg, 2.5 mmol, 1 eq). The mixture was stirred at 30° C. for 12 hr. Then NaBD₄ (286.0 mg, 7.6 mmol, 3 eq) was added to the mixture at 0° C. The mixture was stirred at 20° C. for 3 hr. LC-MS showed desired compound was detected. The reaction mixture was concentrated under reduced pressure to remove solvent. The residue was diluted with H₂O 300 mL and extracted with ethyl acetate (300 mL×3). The combined organic layers were washed with brine (250 mL×1), dried over Na₂SO₄, filtered and concentrated under reduced pressure to give a residue. The residue was purified by flash silica gel chromatography (20 g Silica Flash Column, Eluent of 0-100% Ethyl acetate/Petroleum ether gradient @ 120 mL/min) to give tert-butyl 4-[[4-(cyclopropylamino)-2-methylsulfanyl-pyrimidin-5-yl]methylamino]-4-deuterio-2,3-dihydroquinoline-1-carboxylate (950 mg) as a yellow oil.

To a solution of tert-butyl 4-[[4-(cyclopropylamino)-2-methylsulfanyl-pyrimidin-5-yl]methylamino]-4-deuterio-2,3-dihydroquinoline-1-carboxylate (950 mg, 2.2 mmol, 1 eq) in DCM (10 mL) was added DIEA (1.4 g, 10.7 mmol, 1.9 mL, 5 eq) and bis(trichloromethyl) carbonate (636.9 mg, 2.2 mmol, 1 eq) at 0° C. The mixture was stirred at 20° C. for 2 hr. LC-MS showed desired compound was detected. The residue was diluted with Sat·NaHCO₃ 500 mL and extracted with ethyl acetate (500 mL×3). The combined organic layers were washed with brine (300 mL×1), dried over Na₂SO₄, filtered and concentrated under reduced pressure to give tert-butyl 4-(1-cyclopropyl-7-methylsulfanyl-2-oxo-4H-pyrimido[4,5-d]pyrimidin-S-yl)-4-deuterio-2,3-dihydroquinoline-1-carboxylate (1 g, crude) as a yellow oil.

To a solution of tert-butyl 4-(1-cyclopropyl-7-methylsulfanyl-2-oxo-4H-pyrimido[4,5-d]pyrimidin-S-yl)-4-deuterio-2,3-dihydroquinoline-1-carboxylate (100 mg, 213.4 umol, 1 eq) in DCM (1 mL) was added m-CPBA (65.0 mg, 320.1 umol, 85% purity, 1.5 eq) at 0° C. The mixture was stirred at 20° C. for 2 hr. LC-MS showed desired compound was detected. The reaction mixture was partitioned between Sat·Na₂SO₃ 30 mL and dichloromethane (30×3 mL). The organic phase was separated, washed with Sat·NaHCO₃ (20 mL×3), dried over Na₂SO₄, filtered and concentrated under reduced pressure to give tert-butyl 4-(1-cyclopropyl-7-methylsulfonyl-2-oxo-4H-pyrimido[4,5-d]pyrimidin-S-yl)-4-deuterio-2,3-dihydroquinoline-1-carboxylate (120 mg, crude) as a yellow solid.

To a solution of tert-butyl 4-(1-cyclopropyl-7-methylsulfonyl-2-oxo-4H-pyrimido[4,5-d]pyrimidin-S-yl)-4-deuterio-2,3-dihydroquinoline-1-carboxylate (120 mg, 240.0 umol, 1 eq) in dioxane (0.5 mL) was added TFA (6.8 mg, 59.9 umol, 4.4 uL, 1.5 eq) and 2-(4-aminopyrazol-1-yl)ethanol (6.1 mg, 47.9 umol, 1.2 eq). The mixture was stirred at 100° C. for 12 hr. LC-MS showed desired compound was detected. The reaction mixture was concentrated under reduced pressure to remove solvent. The residue was purified by prep-HPLC (FA condition; column: Phenomenex Luna C18 200*40 mm*10 um; mobile phase: [water(FA)-ACN]; B %: 25%-55%, 8 min) to give tert-butyl 4-[1-cyclopropyl-7-[[1-(2-hydroxyethyl)pyrazol-4-yl]amino]-2-oxo-4H-pyrimido[4,5-d]pyrimidin-S-yl]-4-deuterio-2,3-dihydroquinoline-1-carboxylate (120 mg, 219 umol) as a brown solid.

A mixture of tert-butyl 4-[1-cyclopropyl-7-[[1-(2-hydroxyethyl)pyrazol-4-yl]amino]-2-oxo-4H-pyrimido[4,5-d] pyrimidin-S-yl]-4-deuterio-2,3-dihydroquinoline-1-carboxylate (120 mg, 219 umol, 1 eq) in HCl/EtOAc (1 mL, 4 M) was stirred at 20° C. for 1 hr. LC-MS showed desired compound was detected. The reaction mixture was filtered and concentrated under reduced pressure to give 1-cyclopropyl-S-(4-deuterio-2, 3-dihydro-1H-quinolin-4-yl)-7-[[1-(2-hydroxyethyl) pyrazol-4-yl] amino]-4H-pyrimido [4, 5-d] pyrimidin-2-one (120 mg, crude) as a brown solid.

To a solution of 2-fluoroprop-2-enoic acid (24.2 mg, 268.2 umol, 1 eq) in Pyridine (1 mL) was added EDCI (77.1 mg, 402.2 umol, 1.5 eq) and 1-cyclopropyl-S-(4-deuterio-2,3-dihydro-1H-quinolin-4-yl)-7-[[1-(2-hydroxyethyl) pyrazol-4-yl]amino]-4H-pyrimido[4,5-d]pyrimidin-2-one (120 mg, 268.2 umol, 1 eq). The mixture was stirred at 50° C. for 12 hr. LC-MS showed desired compound was detected. The reaction mixture was concentrated under reduced pressure to remove solvent. The residue was purified by prep-HPLC (FA condition; column: Phenomenex Luna C18 200*40 mm*10 um; mobile phase: [water(FA)-ACN]; B %: 15%-45%, 8 min) to give 1-cyclopropyl-S-[4-deuterio-1-(2-fluoroprop-2-enoyl)-2,3-dihydroquinolin-4-yl]-7-[[1-(2-hydroxyethyl)pyrazol-4-yl]amino]-4H-pyrimido[4,5-d]pyrimidin-2-one (41.3 mg, 79.5 umol, 29.6% yield, 100% purity) as a yellow solid.

Deuteration rate (QTOF-MS): 98.41%. MS (ESI): m/z=520.2 [M+H]⁺

### Procedure for Preparation of Compound 116

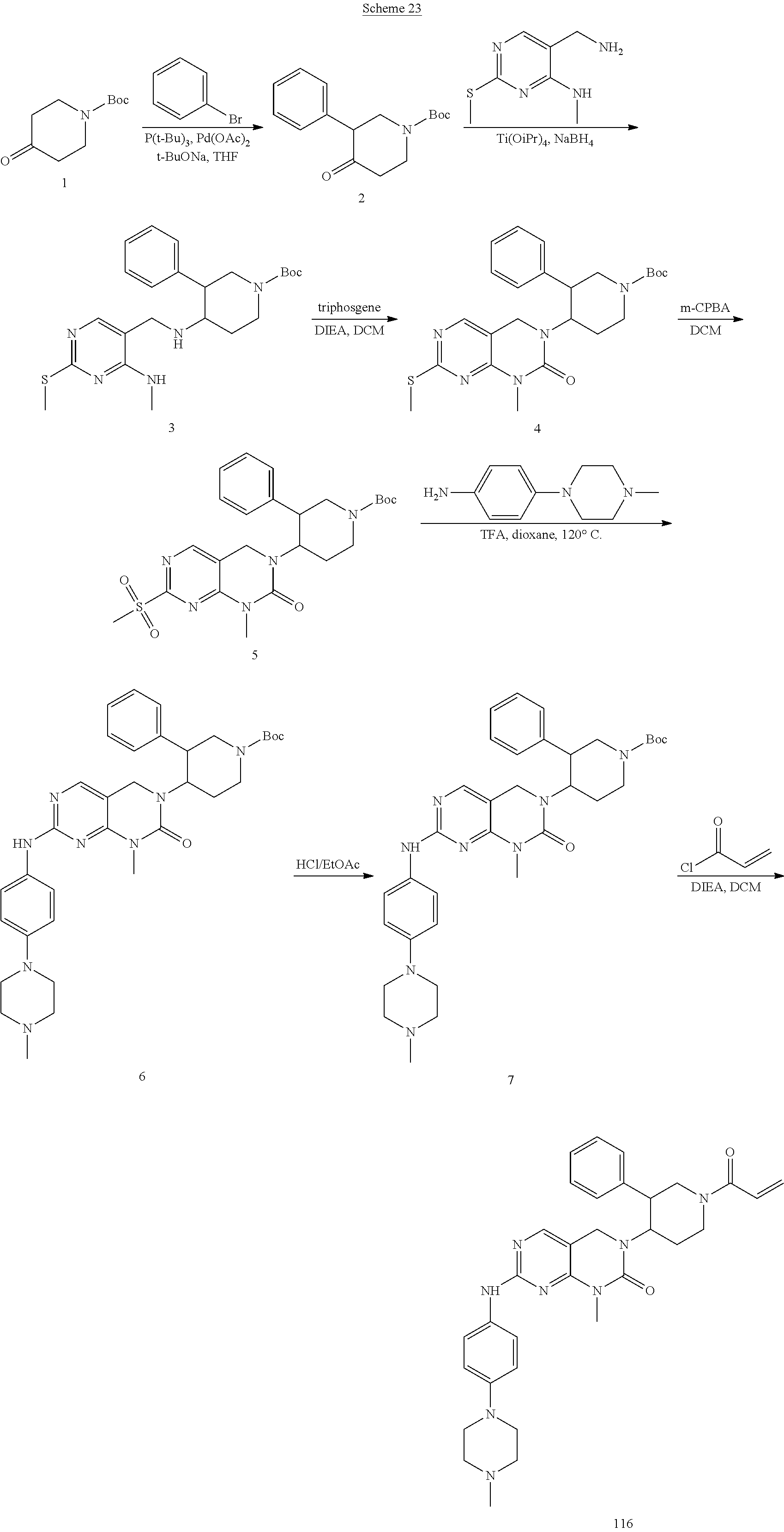

To a solution of tert-butyl 4-oxopiperidine-1-carboxylate (5 g, 25.09 mmol, 1 eq) and bromobenzene (7.88 g, 50.19 mmol, 5.29 mL, 2 eq) in THE (100 mL) was added t-BuONa (3.62 g, 37.64 mmol, 1.5 eq), Pd(OAc)₂ (281.70 mg, 1.25 mmol, 0.05 eq) and t-Bu₃P (330.01 mg, 1.63 mmol, 382.84 uL, 0.065 eq) at 20° C. under N₂. The reaction mixture was evacuated and recharged with N₂ for 3 times and then stirred at 65° C. for 12 hr under N₂. LCMS showed the desired product mass peak was detected. The mixture was concentrated under vacuum. The residue was diluted with 150 mL brine and extracted with EtOAc (300 mL*3). The combined organic phase was dried with anhydrous Na₂SO₄, the mixture was filtered and the filtrate was concentrated under vacuum. The residue was purified by flash silica gel chromatography (ISCO®; 80 g SepaFlash® Silica Flash Column, Eluent of 0-8% Ethyl acetate/Petroleum ether gradient @ 100 mL/min) to give tert-butyl 4-oxo-S-phenyl-piperidine-1-carboxylate (6 g, 21.79 mmol, 43.42% yield) as a yellow oil.

1H NMR (400 MHz, CHLOROFORM-d) δ ppm 7.29-7.40 (m, 3H) 7.19 (br d, J=7.13 Hz, 2 H) 3.93-4.41 (m, 2H) 3.44-3.81 (m, 3H) 2.39-2.77 (m, 2H) 1.42-1.68 (m, 9H)

To a solution of tert-butyl 4-oxo-S-phenyl-piperidine-1-carboxylate (2.5 g, 9.08 mmol, 1 eq) and 5-(aminomethyl)-N-methyl-2-methylsulfanyl-pyrimidin-4-amine (1.67 g, 9.08 mmol, 1 eq) in MeOH (20 mL) was added Ti(i-PrO)₄ (7.74 g, 27.24 mmol, 8.04 mL, 3 eq) at 20° C. under N₂. The mixture was stirred at 30° C. for 12 hr under N₂. Then NaBH₄ (2.06 g, 54.48 mmol, 6 eq) was added to the mixture at 0° C. The mixture was stirred at 20° C. for 12 hr. LCMS showed the desired product mass peak was detected. The reaction mixture was quenched with NH₄Cl solution (30 mL) at 0° C., The reaction mixture was diluted with 50 mL MeOH. The mixture was filtered and the filtrate was concentrated under vacuum. The residue was diluted with 20 mL H₂O and extracted with EtOAc(50 mL*3). The combined organic phase was dried with anhydrous Na₂SO₄, the mixture was filtered and the filtrate was concentrated under vacuum. The residue was purified by flash silica gel chromatography (ISCO; 40 g SepaFlash Silica Flash Column, Eluent of 0-40% Ethyl acetate/Petroleum ether gradient @ 50 mL/min) to give tert-butyl 4-[[4-(methylamino)-2-methylsulfanyl-pyrimidin-5-yl]methylamino]-S-phenyl-piperidine-1-carboxylate (1.4 g, 3.16 mmol, 34.76% yield) as a yellow oil.

To a solution of tert-butyl 4-[[4-(methylamino)-2-methylsulfanyl-pyrimidin-5-yl]methylamino]-S-phenyl-piperidine-1-carboxylate (1.40 g, 3.16 mmol, 1 eq) in DCM(18 mL) was added DIPEA (1.22 g, 9.47 mmol, 1.65 mL, 3 eq) at 0° C. Then bis(trichloromethyl) carbonate (936.53 mg, 3.16 mmol, 1 eq) in DCM(2 mL) was added to the mixture at 0° C. The mixture was stirred at 20° C. for 12 hr. LCMS showed the reaction was completed and desired product mass peak was detected. The reaction mixture was quenched with saturated NaHCO₃ aqueous solution(30 mL) at 0° C., then the mixture was extracted with DCM (50 mL*3). The combined organic phase was dried with anhydrous Na₂SO₄, the mixture was filtered and the filtrate was concentrated under vacuum. The residue was purified by flash silica gel chromatography (ISCO®; 20 g SepaFlash® Silica Flash Column, Eluent of 0-35% Ethyl acetate/Petroleum ether gradient @ 20 mL/min) to give tert-butyl 4-(1-methyl-7-methylsulfanyl-2-oxo-4H-pyrimido[4,5-d]pyrimidin-S-yl)-S-phenyl-piperidine-1-carboxylate (900 mg, 1.92 mmol, 60.73% yield) as a yellow solid.

1H NMR (400 MHz, CHLOROFORM-d) δ ppm 7.45 (s, 1H) 7.27 (br s, 5H) 4.10-4.26 (m, 2H) 3.74-3.87 (m, 2H) 3.47 (br d, J=1.00 Hz, 3H) 2.51-2.66 (m, 3H) 2.04-2.20 (m, 4H) 1.53 (br s, 9H) 1.30-1.34 (m, 2H)

To a solution of tert-butyl 4-(1-methyl-7-methylsulfanyl-2-oxo-4H-pyrimido[4,5-d]pyrimidin-S-yl)-S-phenyl-piperidine-1-carboxylate (900.00 mg, 1.92 mmol, 1 eq) in DCM (10 mL) was added MCPBA (826.82 mg, 4.79 mmol, 2.5 eq) at 0° C. The mixture was stirred at 20° C. for 1.5 hr. LCMS showed the reaction was completed and desired product was detected. The reaction mixture was quenched with saturated Na₂SO₃ aqueous solution(15 mL) at 0° C., then the mixture was extracted with DCM (15 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-45% Ethyl acetate/Petroleum ether gradient @ 15 mL/min) to give tert-butyl 4-(1-methyl-7-methylsulfonyl-2-oxo-4H-pyrimido[4,5-d]pyrimidin-S-yl)-S-phenyl-piperidine-1-carboxylate (550 mg, 1.10 mmol, 57.21% yield) as a white solid.

To a solution of tert-butyl 4-(1-methyl-7-methylsulfonyl-2-oxo-4H-pyrimido[4,5-d]pyrimidin-S-yl)-S-phenyl-piperidine-1-carboxylate (530 mg, 1.06 mmol, 1 eq) and 4-(4-methylpiperazin-1-yl)aniline (606.31 mg, 3.17 mmol, 3 eq) in dioxane (10 mL) was added TFA (240.95 mg, 2.11 mmol, 156.46 uL, 2 eq) at 20° C. under N₂. The mixture was stirred at 120° C. for 12 hr under the N₂. LCMS showed the desired product mass peak was detected. The reaction mixture was concentrated under reduced pressure. The residue was purified by prep-HPLC (FA condition, column: Phenomenex Luna C18 75*30 mm*3 um;mobile phase: [water(FA)-ACN];B %: 20%-50%, 8 min) to give tert-butyl 4-[1-methyl-7-[4-(4-methylpiperazin-1-yl)anilino]-2-oxo-4H-pyrimido[4,5-d]pyrimidin-S-yl]-S-phenyl-piperidine-1-carboxylate (140 mg, 194.62 umol, 18.42% yield, 91.58% purity, FA) as a white solid.

1H NMR (400 MHz, CHLOROFORM-d) δ ppm 8.40 (br s, 1H) 7.46 (d, J=8.76 Hz, 2H) 7.29 (s, 1H) 7.15-7.26 (m, 5H) 6.89-7.01 (m, 3H) 4.83 (dt, J=13.20, 4.22 Hz, 1H) 4.28-4.66 (m, 2H) 3.72 (d, J=13.63 Hz, 1H) 3.41 (s, 3H) 3.28-3.38 (m, 1H) 3.16-3.27 (m, 5H) 2.92-3.02 (m, 1H) 2.70-2.79 (m, 5H) 2.44 (s, 3H) 2.07 (qd, J=12.44, 4.19 Hz, 1H) 1.63 (br d, J=11.38 Hz, 1H) 1.43-1.52 (m, 9H)

To a solution of tert-butyl 4-[1-methyl-7-[4-(4-methylpiperazin-1-yl)anilino]-2-oxo-4H-pyrimido[4,5-d]pyrimidin-S-yl]-S-phenyl-piperidine-1-carboxylate (120.00 mg, 195.83 umol, 1 eq) in HCl/EtOAc (4 mL). The mixture was stirred at 20° C. for 1 hr. LCMS showed the reaction was completed and desired product was detected. The reaction mixture was concentrated under reduced pressure to give 1-methyl-7-[4-(4-methylpiperazin-1-yl)anilino]-3-(3-phenyl-4-piperidyl)-4H-pyrimido[4,5-d]pyrimidin-2-one (100 mg, crude, HCl) as a white solid.

To a solution of 1-methyl-7-[4-(4-methylpiperazin-1-yl)anilino]-S-(3-phenyl-4-piperidyl)-4H-pyrimido[4,5-d]pyrimidin-2-one (100.00 mg, 182.11 umol, 1 eq, HCl) in DCM(2 ml) was added DIPEA (70.61 mg, 546.34 umol, 95.16 uL, 3 eq). The prop-2-enoyl chloride (16.48 mg, 182.11 umol, 14.85 uL, 1 eq) in DCM (0.2 ml) was added to the mixture under N₂ atmosphere at 0° C. The mixture was stirred at 0° C. for 1 hr under N₂. LCMS showed detection of the desired product mass peak. The reaction mixture was concentrated under reduced pressure. The residue was purified by prep-HPLC (FA condition; column: Phenomenex Luna C18 75*30 mm*3 um;mobile phase: [water(FA)-ACN];B %: 1%-30%, 8 min) to give 1-methyl-7-[4-(4-methylpiperazin-1-yl)anilino]-S-(3-phenyl-1-prop-2-enoyl-4-piperidyl)-4H-pyrimido[4,5-d]pyrimidin-2-one (22.6 mg, 36.88 umol, 20.25% yield, 100% purity, FA) as a white solid. MS (ESI): m/z=567.2 [M+H]⁺

TABLE 2

| Compound | | LCMS | |
|---|---|---|---|
| No. | Structure | (M + H) | 1H NMR (DMSO unless stated) |
| 001 | 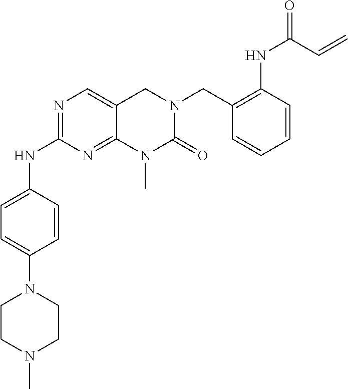 [SMILES: C=CC(=O)Nc1ccccc1CN1Cc2cnc(Nc3ccc(N4CCN(C)CC4)cc3)nc2N(C)C1=O] | 513.2 | |
| 036 | 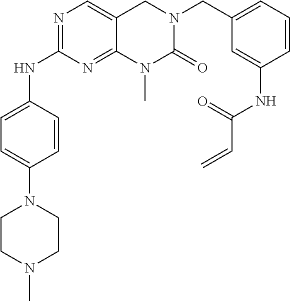 [SMILES: C=CC(=O)Nc1cccc(CN2Cc3cnc(Nc4ccc(N5CCN(C)CC5)cc4)nc3N(C)C2=O)c1] | 513.2 | δ: 10.17 (s, 1H), 9.22 (s, 1H), 8.20 (s, 1H), 8.00 (s, 1H), 7.68 (br d, J = 8.3 Hz, 1H), 7.58 − 7.53 (m, 3H), 7.31 (t, J = 7.8 Hz, 1H), 7.03 (d, J = 7.6 Hz, 1H), 6.87 (d, J = 9.1 Hz, 2H), 6.46 − 6.37 (m, 1H), 6.28 − 6.21 (m, 1H), 5.77 − 5.71 (m, 1H), 4.57 (s, 2H), 4.23 (s, 2H), 3.31 (s, 3H), 3.08 − 3.03 (m, 4H), 2.47 (br d, J = 4.9 Hz, 4H), 2.23 (s, 3H) |
| 028 | 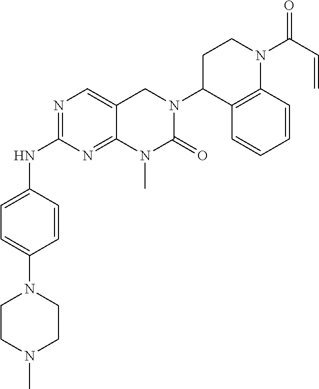 [SMILES: C=CC(=O)N1CCC(N2Cc3cnc(Nc4ccc(N5CCN(C)CC5)cc4)nc3N(C)C2=O)c2ccccc21] | 539.2 | δ: 8.96 − 8.80 (m, 1H), 7.99 − 7.84 (m, 1H), 7.59 − 7.43 (m, 2H), 7.30 − 7.15 (m, 3H), 6.86 (d, J = 9.0 Hz, 2H), 6.72 − 6.51 (m, 1H), 6.29 − 6.12 (m, 1H), 5.78 − 5.68 (m, 1H), 5.68 − 5.50 (m, 1H), 4.25 − 4.05 (m, 2H), 3.96 − 3.82 (m, 1H), 3.80 − 3.69 (m, 1H), 3.62 − 3.49 (m, 1H), 3.34 (s, 3H), 3.10 − 3.04 (m, 4H), 2.46 (br d, J = 4.9 Hz, 4H), 2.28 − 2.17 (m, 4H), 2.15 − 1.96 (m, 1H) |
| 041 | 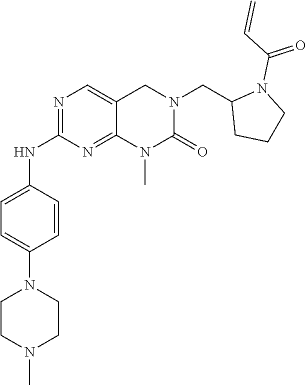 [SMILES: C=CC(=O)N1CCCC1CN1Cc2cnc(Nc3ccc(N4CCN(C)CC4)cc3)nc2N(C)C1=O] | 491.2 | δ: 9.28 − 9.15 (m, 1H), 8.24 − 8.15 (m, 1H), 8.10 − 7.98 (m, 1H), 7.60 − 7.44 (m, 2H), 6.88 (d, J = 8.4 Hz, 2H), 6.81 − 6.53 (m, 1H), 6.19 − 6.02 (m, 1H), 5.75 − 5.47 (m, 1H), 4.48 − 4.29 (m, 3H), 3.68 − 3.61 (m, 2H), 3.37 − 3.29 (m, 2H), 3.27 − 3.23 (m, 3H), 3.09 − 3.01 (m, 4H), 2.50 − 2.46 (m, 4H), 2.27 − 2.20 (m, 3H), 2.06 − 1.72 (m, 4H). |
| 039 | 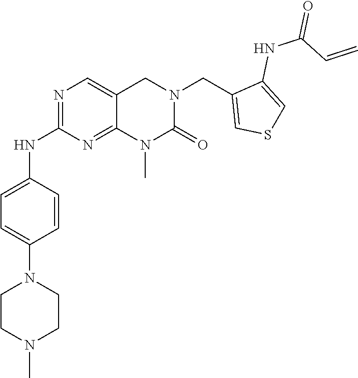 [SMILES: C=CC(=O)Nc1cscc1CN1Cc2cnc(Nc3ccc(N4CCN(C)CC4)cc3)nc2N(C)C1=O] | 519.1 | δ: 10.12 (s, 1H), 9.25 (s, 1H), 8.03 (s, 1H), 7.81 (d, J = 3.1 Hz, 1H), 7.58 − 7.46 (m, 3H), 6.91 − 6.82 (m, 2H), 6.50 − 6.23 (m, 2H), 5.80 (d, J = 10.0 Hz, 1H), 4.57 (s, 2H), 4.31 (s, 2H), 3.30 (s, 3H), 3.04 (br s, 4H), 2.45 (br s, 4H), 2.22 (s, 3H) |
| 037 | 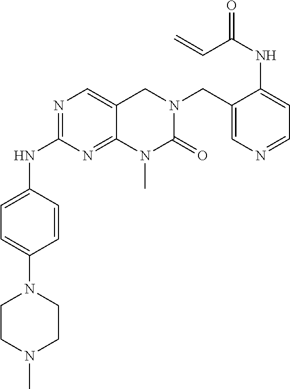 [SMILES: C=CC(=O)Nc1ccncc1CN1Cc2cnc(Nc3ccc(N4CCN(C)CC4)cc3)nc2N(C)C1=O] | 514.2 | δ: 2.19 − 2.23 (m, 3 H) 2.41 − 2.45 (m, 4 H) 3.02 − 3.08 (m, 4 H) 3.31 (s, 3 H) 4.32 (s, 2 H) 4.67 (s, 2 H) 5.86 − 5.94 (m, 1 H) 6.31 − 6.40 (m, 1 H) 6.47 − 6.59 (m, 1 H) 6.80 − 6.91 (m, 2 H) 7.53 (d, J = 9.01 Hz, 2 H) 8.03 (s, 1 H) 8.17 − 8.23 (m, 1 H) 8.30 − 8.35 (m, 1 H) 8.46 (d, J = 5.63 Hz, 1 H) 8.53 (s, 1 H) 9.20 − 9.28 (m, 1 H) 10.26 − 10.33 (m, 1 H) |
| 031 | 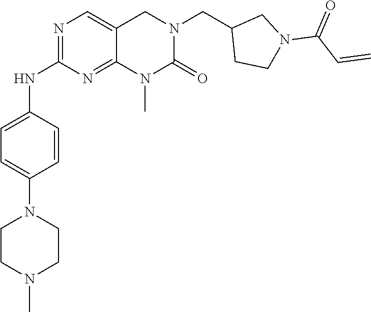 [SMILES: C=CC(=O)N1CCC(CN2Cc3cnc(Nc4ccc(N5CCN(C)CC5)cc4)nc3N(C)C2=O)C1] | 491.2 | δ: 9.21 (s, 1H), 8.21 (s, 1H), 8.05 − 8.00 (m, 1H), 7.56 (d, J = 9.0 Hz, 2H), 6.87 (d, J = 9.0 Hz, 2H), 6.55 (ddd, J = 4.9, 10.3, 16.7 Hz, 1H), 6.16 − 6.07 (m, 1H), 5.64 (ddd, J = 2.4, 7.2, 10.1 Hz, 1H), 4.34 (br s, 2H), 3.55 − 3.47 (m, 3H), 3.43 − 3.38 (m, 2H), 3.25 (s, 3H), 3.13 − 3.03 (m, 5H), 2.66 − 2.53 (m, 1H), 2.47 (br s, 4H), 2.24 (s, 3H), 2.05 − 1.87 (m, 1H), 1.74 − 1.52 (m, 1H) |
| 042 | 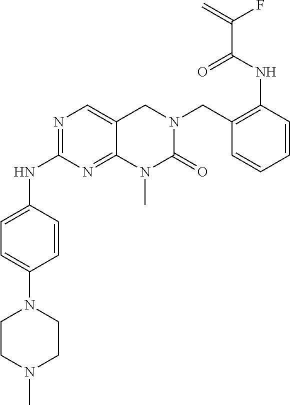 [SMILES: C=C(F)C(=O)Nc1ccccc1CN1Cc2cnc(Nc3ccc(N4CCN(C)CC4)cc3)nc2N(C)C1=O] | 531.1 | δ: 10.32 (s, 1H), 9.24 − 9.20 (m, 1H), 8.21 (s, 1H), 8.01 − 7.98 (m, 1H), 7.58 − 7.52 (m, 2H), 7.48 − 7.44 (m, 1H), 7.39 − 7.26 (m, 3H), 6.90 − 6.83 (m, 2H), 5.78 − 5.60 (m, 1H), 5.41 (dd, J = 3.6, 15.6 Hz, 1H), 4.56 (s, 2H), 4.29 − 4.24 (m, 2H), 3.27 (s, 3H), 3.06 − 3.03 (m, 4H), 2.47 − 2.43 (m, 4H), 2.22 (s, 3H) |
| 019 | 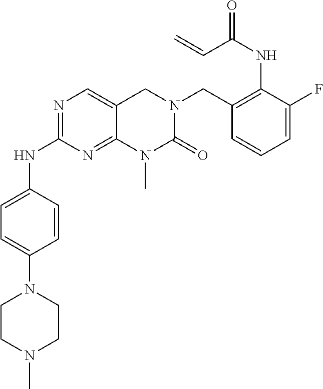 [SMILES: C=CC(=O)Nc1c(F)cccc1CN1Cc2cnc(Nc3ccc(N4CCN(C)CC4)cc3)nc2N(C)C1=O] | 531.2 | δ: 10.49 − 10.48 (m, 1H), 9.95 − 9.91 (s, 1H), 9.84 − 9.81 (m, 1H), 8.01 (s, 1H), 7.59 − 7.51 (m, 2H), 7.34 (dt, J = 5.6, 7.9 Hz, 1H), 7.29 − 7.21 (m, 1H), 7.15 (br d, J = 7.8 Hz, 1H), 7.04 − 6.94 (m, 2H), 6.53 − 6.41 (m, 1H), 6.22 (dd, J = 1.8, 17.1 Hz, 1H), 5.82 − 5.73 (m, 1H), 4.57 (s, 2H), 4.23 (s, 2H), 3.76 (br d, J = 12.8 Hz, 2H), 3.29 (s, 3H), 3.23 − 3.10 (m, 4H), 3.07 − 2.99 (m, 2H), 2.83 (br d, J = 4.1 Hz, 3H). |
| 031 | 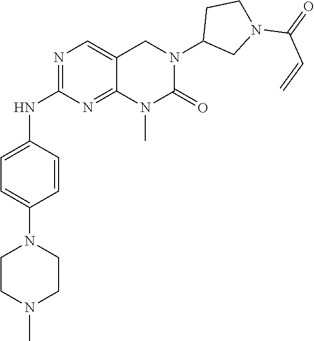 [SMILES: C=CC(=O)N1CCC(N2Cc3cnc(Nc4ccc(N5CCN(C)CC5)cc4)nc3N(C)C2=O)C1] | 477.1 | δ: 9.22 (br d, J = 1.8 Hz, 1H), 8.09 − 8.01 (m, 1H), 7.55 (d, J = 9.0 Hz, 2H), 6.92 − 6.82 (m, 2H), 6.66 − 6.53 (m, 1H), 6.21 − 6.08 (m, 1H), 5.69 (dt, J = 2.3, 10.0 Hz, 1H), 5.00 − 4.81 (m, 1H), 4.29 (br d, J = 6.9 Hz, 2H), 3.82 − 3.76 (m, 1H), 3.68 − 3.59 (m, 3H), 3.27 (s, 3H), 3.08 − 3.02 (m, 4H), 2.47 − 2.42 (m, 4H), 2.21 (s, 3H), 2.18 − 2.02 (m, 2H) |
| 032 | 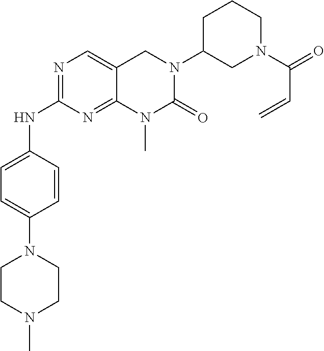 [SMILES: C=CC(=O)N1CCCC(N2Cc3cnc(Nc4ccc(N5CCN(C)CC5)cc4)nc3N(C)C2=O)C1] | 491.2 | δ: 9.22 (s, 1H), 8.02 (s, 1H), 7.55 (d, J = 8.9 Hz, 2H), 6.87 (d, J = 9.0 Hz, 2H), 6.83 − 6.70 (m, 1H), 6.10 (dd, J = 2.1, 16.7 Hz, 1H), 5.76 − 5.61 (m, 1H), 4.46 − 4.26 (m, 3H), 4.11 − 3.95 (m, 2H), 3.26 (s, 3H), 3.07 − 3.02 (m, 4H), 2.46 − 2.41 (m, 4H), 2.21 (s, 3H), 1.96 − 1.85 (m, 1H), 1.84 − 1.72 (m, 2H), 1.50 − 1.36 (m, 1H) |
| 012 | 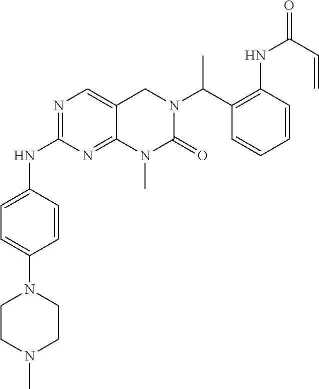 [SMILES: C=CC(=O)Nc1ccccc1C(C)N1Cc2cnc(Nc3ccc(N4CCN(C)CC4)cc3)nc2N(C)C1=O] | 527.2 | δ: 9.61 − 9.54 (m, 1H), 9.21 − 9.13 (m, 1H), 7.93 (s, 1H), 7.65 (br d, J = 7.9 Hz, 1H), 7.57 − 7.50 (m, 3H), 7.39 − 7.21 (m, 2H), 6.90 − 6.81 (m, 2H), 6.43 − 6.31 (m, 1H), 6.16 (dd, J = 1.8, 17.0 Hz, 1H), 5.96 − 5.86 (m, 1H), 5.78 − 5.69 (m, 1H), 4.27 − 4.17 (m, 1H), 3.62 (d, J = 14.3 Hz, 1H), 3.31 − 3.29 (m, 2H), 3.24 (s, 3H), 3.06 − 3.01 (m, 4H), 2.70 − 2.64 (m, 1H), 2.35 − 2.30 (m, 1H), 2.21 (s, 3H), 1.52 (d, J = 7.0 Hz, 3H) |
| 046 | 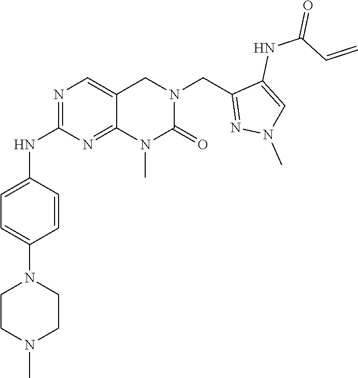 [SMILES: C=CC(=O)Nc1cn(C)nc1CN1Cc2cnc(Nc3ccc(N4CCN(C)CC4)cc3)nc2N(C)C1=O] | 517.2 | δ: 9.70 (s, 1 H) 9.18 − 9.26 (m, 1 H) 8.22 (s, 1 H) 8.06 (d, J = 4.13 Hz, 2 H) 7.55 (d, J = 9.13 Hz, 2 H) 6.87 (d, J = 9.01 Hz, 2 H) 6.30 − 6.44 (m, 1 H) 6.17 − 6.27 (m, 1 H) 5.68 − 5.86 (m, 1 H) 4.52 − 4.65 (m, 2 H) 4.26 − 4.40 (m, 2 H) 3.73 − 3.85 (m, 3 H) 3.28 (s, 3 H) 2.98 − 3.08 (m, 4 H) 2.41 − 2.47 (m, 4 H) 2.22 (s, 3 H) |
| 047 | 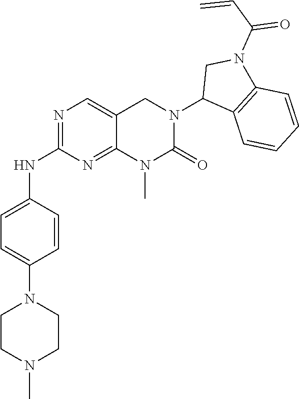 [SMILES: C=CC(=O)N1CC(N2Cc3cnc(Nc4ccc(N5CCN(C)CC5)cc4)nc3N(C)C2=O)c2ccccc21] | 525.3 | δ: 9.13 − 9.27 (m, 1 H) 8.24 − 8.33 (m, 1 H) 8.16 − 8.23 (m, 1 H) 7.91 − 8.00 (m, 1 H) 7.48 − 7.60 (m, 2 H) 7.26 − 7.41 (m, 2 H) 7.08 − 7.18 (m, 1 H) 6.83 − 6.91 (m, 2 H) 6.70 − 6.82 (m, 1 H) 6.27 − 6.39 (m, 1 H) 6.11 − 6.26 (m, 1 H) 5.78 − 5.90 (m, 1 H) 4.39 − 4.54 (m, 1 H) 4.23 − 4.33 (m, 1 H) 4.11 − 4.21 (m, 1 H) 3.70 − 3.70 (m, 1 H) 3.68 (br d, J = 13.63 Hz, 1 H) 3.29 − 3.34 (m, 3 H) 2.99 − 3.09 (m, 4 H) 2.40 − 2.47 (m, 4 H) 2.16 − 2.28 (m, 3 H) |
| 048 | 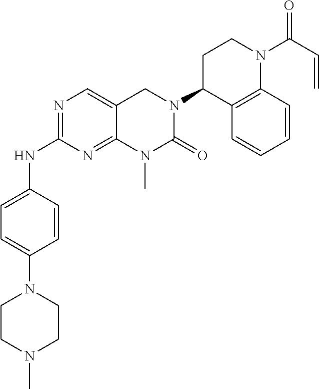 [SMILES: C=CC(=O)N1CC[C@H](N2Cc3cnc(Nc4ccc(N5CCN(C)CC5)cc4)nc3N(C)C2=O)c2ccccc21] | 539.2 | δ: 9.23 (s, 1 H) 7.97 (s, 1 H) 7.55 (d, J = 9.01 Hz, 2 H) 7.21 − 7.32 (m, 2 H) 7.13 − 7.21 (m, 2 H) 6.83 − 6.91 (m, 2 H) 6.65 (br dd, J = 16.76, 10.38 Hz, 1 H) 6.26 (dd, J = 16.76, 1.88 Hz, 1 H) 5.77 (dd, J = 10.32, 1.94 Hz, 1 H) 5.61 − 5.70 (m, 1 H) 4.08 − 4.31 (m, 2 H) 3.83 − 3.95 (m, 1 H) 3.58 − 3.78 (m, 1 H) 2.98 − 3.10 (m, 4 H) 2.39 − 2.47 (m, 4 H) 2.14 − 2.28 (m, 4 H) 1.96 − 2.12 (m, 1 H) |
| 049 | 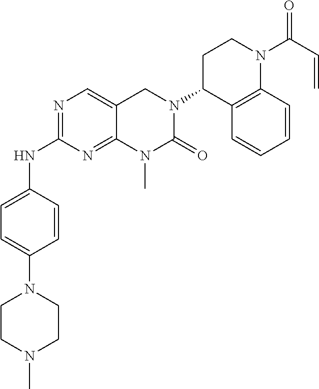 [SMILES: C=CC(=O)N1CC[C@@H](N2Cc3cnc(Nc4ccc(N5CCN(C)CC5)cc4)nc3N(C)C2=O)c2ccccc21] | 539.2 | δ: 9.24 (s, 1H), 8.01 − 7.94 (m, 1H), 7.56 (d, J = 9.0 Hz, 2H), 7.33 − 7.21 (m, 2H), 7.19 (br d, J = 3.9 Hz, 2H), 6.92 − 6.84 (m, 2H), 6.65 (br dd, J = 10.4, 16.8 Hz, 1H), 6.32 − 6.23 (m, 1H), 5.78 (dd, J = 1.9, 10.4 Hz, 1H), 5.71 − 5.62 (m, 1H), 4.28 − 4.13 (m, 2H), 3.95 − 3.87 (m, 1H), 3.79 − 3.65 (m, 1H), 3.33 (s, 3H), 3.10 − 3.01 (m, 4H), 2.49 − 2.40 (m, 4H), 2.27 − 2.16 (m, 4H), 2.12 − 2.00 (m, 1H) |
| 050 | 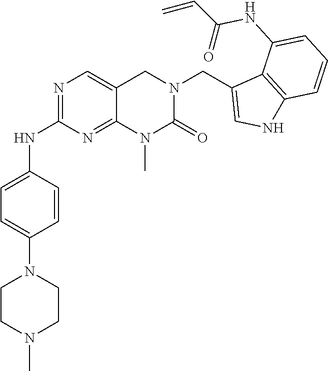 [SMILES: C=CC(=O)Nc1cccc2[nH]cc(CN3Cc4cnc(Nc5ccc(N6CCN(C)CC6)cc5)nc4N(C)C3=O)c12] | 552.3 | δ: 11.37 − 11.24 (m, 1H), 10.29 − 10.14 (m, 1H), 9.95 − 9.71 (m, 1H), 9.58 − 9.45 (m, 1H), 7.99 (s, 1H), 7.57 (d, J = 8.9 Hz, 2H), 7.46 − 7.39 (m, 1H), 7.32 (br d, J = 7.5 Hz, 1H), 7.23 (d, J = 7.9 Hz, 1H), 7.06 (t, J = 7.9 Hz, 1H), 6.96 (d, J = 9.0 Hz, 2H), 6.61 (br dd, J = 10.2, 17.1 Hz, 1H), 6.28 − 6.18 (m, 1H), 5.75 (br d, J = 11.0 Hz, 1H), 4.82 (s, 2H), 4.28 (s, 2H), 3.56 − 3.41 (m, 4H), 3.30 − 3.26 (m, 3H), 3.21 − 3.09 (m, 3H), 2.86 (s, 4H) |
| 051 | 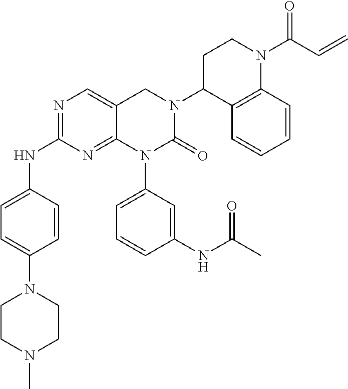 [SMILES: C=CC(=O)N1CCC(N2Cc3cnc(Nc4ccc(N5CCN(C)CC5)cc4)nc3N(c3cccc(NC(C)=O)c3)C2=O)c2ccccc21] | 658.2 | δ: 10.17 (s, 1 H) 9.11 − 9.28 (m, 1 H) 8.04 − 8.15 (m, 1 H) 7.92 − 8.01 (m, 1 H) 7.68 − 7.77 (m, 1 H) 7.57 − 7.63 (m, 1 H) 7.45 (t, J = 8.01 Hz, 1 H) 7.29 − 7.35 (m, 2 H) 7.22 − 7.29 (m, 2 H) 7.16 (br d, J = 7.46 Hz, 2 H) 7.00 − 7.09 (m, 1 H) 6.62 − 6.73 (m, 1 H) 6.55 − 6.60 (m, 1 H) 6.22 − 6.35 (m, 1 H) 5.75 − 5.85 (m, 1 H) 5.69 (br t, J = 8.25 Hz, 1 H) 4.43 (d, J = 14.18 Hz, 1 H) 4.13 − 4.27 (m, 1 H) 4.00 − 4.12 (m, 1 H) 3.67 − 3.84 (m, 1 H) 3.02 (br s, 4 H) 2.91 (s, 3 H) 2.75 (s, 3 H) 2.54 − 2.59 (m, 4 H) 2.23 − 2.28 (m, 1 H) 2.10 − 2.21 (m, 1 H) |
| 052 | 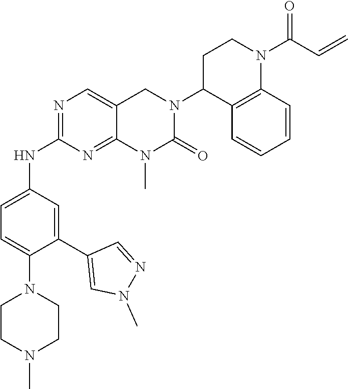 | 619.4 | δ: 9.40 − 9.34 (m, 1H), 8.14 − 8.11 (m, 1H), 8.04 − 8.00 (m, 1H), 7.91 − 7.86 (m, 1H), 7.84 − 7.80 (m, 1H), 7.56 (dd, J = 2.4, 8.8 Hz, 1H), 7.30 − 7.22 (m, 2H), 7.19 (br s, 1H), 7.06 (d, J = 8.6 Hz, 1H), 6.69 − 6.60 (m, 1H), 6.30 − 6.22 (m, 1H), 5.81 − 5.76 (m, 1H), 5.70 − 5.64 (m, 1H), 5.42 − 5.36 (m, 1H), 4.27 − 4.14 (m, 2H), 3.90 (s, 4H), 3.75 − 3.68 (m, 1H), 3.35 (br s, 3H), 2.88 (br s, 4H), 2.48 − 2.45 (m, 4H), 2.21 − 2.16 (m, 1H), 2.07 (s, 1H), 1.23 (s, 3H) |
| 053 | 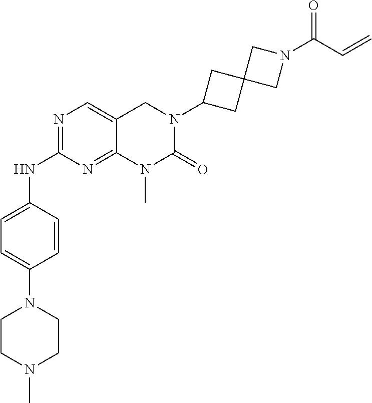 [SMILES: C=CC(=O)N1CC2(CC(N3Cc4cnc(Nc5ccc(N6CCN(C)CC6)cc5)nc4N(C)C3=O)C2)C1] | 503.2 | δ: 9.20 (s, 1H), 8.20 − 8.16 (m, 1H), 8.06 (s, 1H), 7.59 − 7.52 (m, 2H), 6.89 − 6.84 (m, 2H), 6.33 − 6.22 (m, 1H), 6.08 (dd, J = 1.6, 16.9 Hz, 1H), 5.68 − 5.62 (m, 1H), 4.59 − 4.47 (m, 1H), 4.30 (s, 1H), 4.27 − 4.22 (m, 2H), 4.17 − 4.14 (m, 1H), 4.03 − 3.99 (m, 1H), 3.90 − 3.86 (m, 2H), 3.26 (s, 3H), 3.14 − 3.03 (m, 4H), 2.49 − 2.33 (m, 8H), 2.23 (s, 3H) |
| 054 | 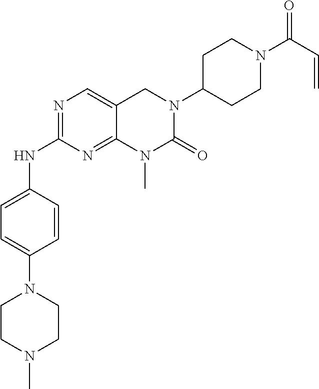 [SMILES: C=CC(=O)N1CCC(N2Cc3cnc(Nc4ccc(N5CCN(C)CC5)cc4)nc3N(C)C2=O)CC1] | 491.2 | δ: 9.26 − 9.19 (m, 1H), 8.19 (s, 1H), 8.04 − 7.99 (m, 1H), 7.59 − 7.47 (m, 2H), 6.98 − 6.79 (m, 3H), 6.11 (dd, J = 2.4, 16.6 Hz, 1H), 5.74 − 5.64 (m, 1H), 4.55 (br d, J = 12.5 Hz, 1H), 4.46 − 4.38 (m, 2H), 4.20 (s, 3H), 3.25 (s, 3H), 3.17 − 3.11 (m, 2H), 3.08 (br s, 4H), 2.62 − 2.55 (m, 4H), 2.30 − 2.27 (m, 2H), 1.65 (br d, J = 5.5 Hz, 4H) |
| 055 | 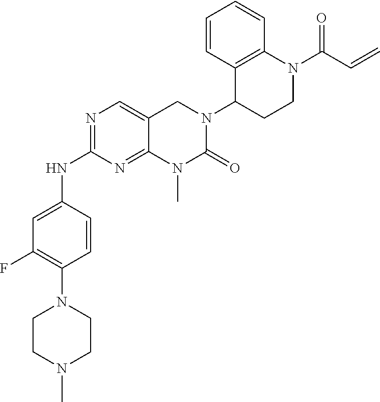 [SMILES: C=CC(=O)N1CCC(N2Cc3cnc(Nc4ccc(N5CCN(C)CC5)c(F)c4)nc3N(C)C2=O)c2ccccc21] | 557.1 | δ: 9.56 − 9.47 (m, 1H), 8.19 − 8.14 (m, 1H), 8.05 − 7.98 (m, 1H), 7.72 − 7.61 (m, 1H), 7.44 − 7.38 (m, 1H), 7.32 − 7.21 (m, 2H), 7.20 − 7.14 (m, 2H), 7.02 − 6.93 (m, 1H), 6.72 − 6.59 (m, 1H), 6.32 − 6.21 (m, 1H), 5.82 − 5.73 (m, 1H), 5.71 − 5.64 (m, 1H), 4.29 − 4.22 (m, 1H), 4.21 − 4.14 (m, 1H), 3.97 − 3.89 (m, 1H), 3.77 − 3.66 (m, 1H), 3.34 (s, 3H), 2.94 (br t, J = 4.1 Hz, 4H), 2.47 (br s, 4H), 2.28 − 2.21 (m, 3H), 2.21 − 2.15 (m, 1H), 2.13 − 1.99 (m, 1H) |
| 056 | 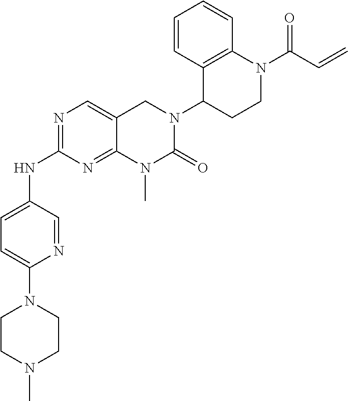 | 540.2 | δ: 2.01 − 2.10 (m, 1 H) 2.14 − 2.19 (m, 1 H) 2.22 (s, 3 H) 2.40 (br t, J = 4.82 Hz, 4 H) 3.34 − 3.41 (m, 7 H) 3.67 − 3.75 (m, 1 H) 3.90 (d, J = 14.01 Hz, 1 H) 4.12 − 4.25 (m, 2 H) 5.66 (dd, J = 9.13, 7.50 Hz, 1 H) 5.74 − 5.80 (m, 1 H) 6.26 (dd, J = 16.76, 2.00 Hz, 1 H) 6.64 (br dd, J = 16.76, 10.51 Hz, 1 H) 6.81 (d, J = 9.01 Hz, 1 H) 7.18 (d, J = 4.00 Hz, 2 H) 7.21 − 7.31 (m, 2 H) 7.85 (dd, J = 9.07, 2.69 Hz, 1 H) 7.97 (s, 1 H) 8.43 (d, J = 2.38 Hz, 1 H) 9.24 (s, 1 H) |
| 057 | 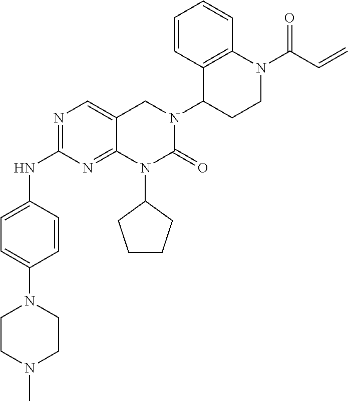 [SMILES: C=CC(=O)N1CCC(N2Cc3cnc(Nc4ccc(N5CCN(C)CC5)cc4)nc3N(C3CCCC3)C2=O)c2ccccc21] | 593.2 | δ: 9.16 − 9.30 (m, 1 H) 8.00 − 8.10 (m, 1 H) 7.56 (d, J = 9.01 Hz, 2 H) 7.22 − 7.42 (m, 3 H) 7.13 − 7.21 (m, 1 H) 6.93 (d, J = 9.01 Hz, 2 H) 6.70 (dd, J = 16.70, 10.32 Hz, 1 H) 6.32 (dd, J = 16.82, 2.06 Hz, 1 H) 5.79 − 5.90 (m, 1 H) 5.63 − 5.73 (m, 1 H) 5.03 − 5.35 (m, 1 H) 4.13 − 4.29 (m, 2 H) 3.86 − 3.97 (m, 1 H) 3.73 − 3.84 (m, 1 H) 3.00 − 3.20 (m, 5 H) 2.58 (br d, J = 2.00 Hz, 4 H) 2.29 - 2.36 (m, 3 H) 2.18 − 2.28 (m, 3 H) 2.09 (ddt, J = 18.29, 10.13, 5.05, 5.05 Hz, 1 H) 1.84 − 1.99 (m, 4 H) 1.54 − 1.67 (m, 2 H) |
| 058 | 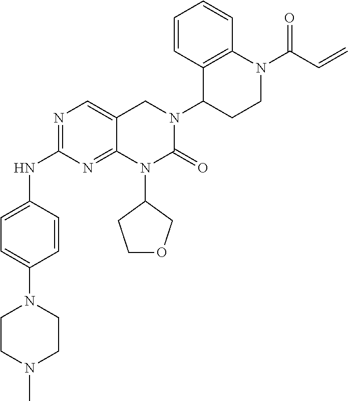 [SMILES: C=CC(=O)N1CCC(N2Cc3cnc(Nc4ccc(N5CCN(C)CC5)cc4)nc3N(C3CCOC3)C2=O)c2ccccc21] | 595.2 | δ: 9.21 (s, 1H), 8.21 (s, 1H), 8.01 (d, J = 1.0 Hz, 1H), 7.51 (d, J = 9.0 Hz, 2H), 7.33 − 7.10 (m, 4H), 6.87 (d, J = 9.0 Hz, 2H), 6.64 (dd, J = 10.3, 16.6 Hz, 1H), 6.26 (dd, J = 2.1, 16.7 Hz, 1H), 5.80 − 5.74 (m, 1H), 5.68 − 5.60 (m, 1H), 5.44 − 5.32 (m, 1H), 4.20 − 4.10 (m, 2H), 4.08 − 3.92 (m, 2H), 3.92 − 3.81 (m, 3H), 3.78 − 3.66 (m, 1H), 3.09 − 3.01 (m, 4H), 2.48 − 2.41 (m, 4H), 2.40 − 2.32 (m, 1H), 2.25 − 2.11 (m, 5H), 2.08 − 1.97 (m, 1H) |
| 059 | 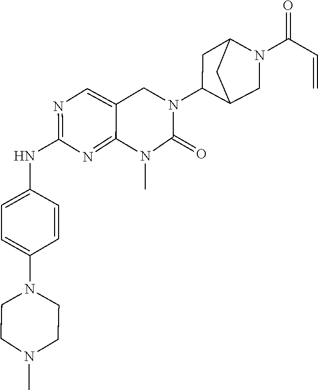 [SMILES: C=CC(=O)N1CC2CC1CC2N1Cc2cnc(Nc3ccc(N4CCN(C)CC4)cc3)nc2N(C)C1=O] | 503.2 | δ: 9.27 (br d, J = 13.2 Hz, 1H), 8.14 (s, 1H), 8.02 − 7.91 (m, 1H), 7.56 (dd, J = 3.7, 8.9 Hz, 2H), 6.88 (br d, J = 8.9 Hz, 2H), 6.68 (dd, J = 10.3, 16.7 Hz, 1H), 6.45 (dd, J = 10.3, 16.7 Hz, 1H), 6.21 − 6.10 (m, 1H), 5.71 − 5.61 (m, 1H), 4.54 − 4.46 (m, 1H), 4.45 − 4.30 (m, 1H), 4.29 − 4.20 (m, 1H), 3.93 (br dd, J = 4.1, 13.6 Hz, 1H), 3.27 − 3.16 (m, 5H), 3.08 (br s, 4H), 2.91 − 2.78 (m, 1H), 2.62 − 2.53 (m, 4H), 2.31 (br s, 3H), 2.20 − 1.98 (m, 1H), 1.85 − 1.57 (m, 3H) |
| 060 | 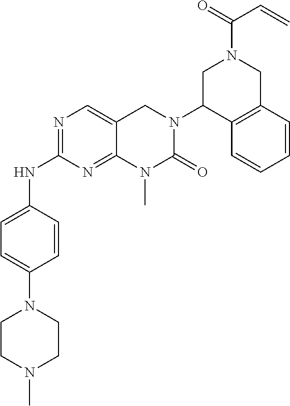 [SMILES: C=CC(=O)N1Cc2ccccc2C(N2Cc3cnc(Nc4ccc(N5CCN(C)CC5)cc4)nc3N(C)C2=O)C1] | 539.2 | δ: 2.21 (s, 3 H) 2.42 − 2.47 (m, 4 H) 3.01 − 3.07 (m, 4 H) 3.33 (s,3 H) 3.73 − 3.95 (m, 2 H) 4.01 (br d, J = 5.63 Hz, 2 H) 4.74 − 4.93 (m, 2 H) 5.54 (br d, J = 4.75 Hz, 1 H) 5.64 − 5.78 (m, 1 H) 6.04 − 6.19 (m, 1 H) 6.76 − 6.98 (m, 3 H) 7.18 − 7.37 (m,4 H) 7.55 (d, J = 8.88 Hz, 2 H) 7.85 (s, 1 H) 9.22 (s, 1 H) |
| 061 | 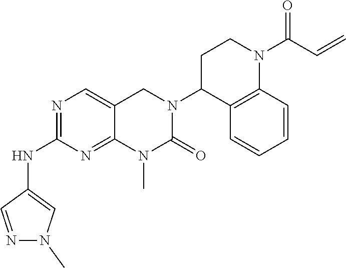 [SMILES: C=CC(=O)N1CCC(N2Cc3cnc(Nc4cnn(C)c4)nc3N(C)C2=O)c2ccccc21] | 445.1 | δ: 9.39 − 9.26 (m, 1H), 8.00 − 7.92 (m, 1H), 7.85 − 7.74 (m, 1H), 7.48 (s, 1H), 7.31 − 7.21 (m, 2H), 7.18 (d, J = 4.3 Hz, 2H), 6.72 − 6.58 (m, 1H), 6.29 − 6.20 (m, 1H), 5.83 − 5.73 (m, 1H), 5.71 − 5.61 (m, 1H), 4.25 − 4.12 (m, 2H), 3.94 − 3.85 (m, 1H), 3.79 (s, 3H), 3.76 − 3.66 (m, 1H), 3.34 (br s, 3H), 2.24 − 2.15 (m, 1H), 2.12 − 1.99 (m, 1H) |
| 062 | 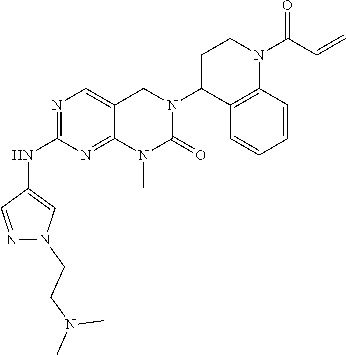 [SMILES: C=CC(=O)N1CCC(N2Cc3cnc(Nc4cnn(CCN(C)C)c4)nc3N(C)C2=O)c2ccccc21] | 502.2 | δ: 9.40 − 9.28 (m, 1H), 8.20 − 8.14 (m, 1H), 8.02 − 7.94 (m, 1H), 7.93 − 7.85 (m, 1H), 7.51 − 7.45 (m, 1H), 7.31 − 7.23 (m, 2H), 7.20 − 7.11 (m, 2H), 6.72 − 6.58 (m, 1H), 6.30 − 6.21 (m, 1H), 5.82 − 5.74 (m, 1H), 5.71 − 5.61 (m, 1H), 4.24 − 4.12 (m, 4H), 3.95 − 3.85 (m, 1H), 3.77 − 3.67 (m, 1H), 3.32 − 3.23 (m, 3H), 2.60 (br t, J = 5.9 Hz, 3H), 2.16 (s, 7H), 2.11 − 2.01 (m, 1H) |
| 063 | 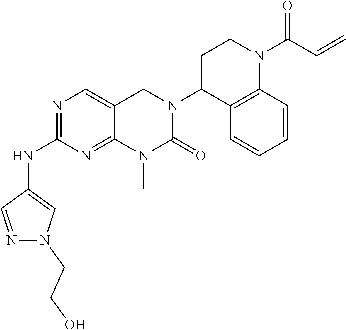 [SMILES: C=CC(=O)N1CCC(N2Cc3cnc(Nc4cnn(CCO)c4)nc3N(C)C2=O)c2ccccc21] | 475.1 | δ: 9.43 − 9.25 (m, 1H), 7.97 (s, 1H), 7.92 − 7.84 (m, 1H), 7.56 − 7.47 (m, 1H), 7.35 − 7.21 (m, 2H), 7.18 (br d, J = 3.9 Hz, 2H), 6.74 − 6.57 (m, 1H), 6.34 − 6.20 (m, 1H), 5.85 − 5.74 (m, 1H), 5.73 − 5.61 (m, 1H), 5.00 − 4.73 (m, 1H), 4.29 − 4.12 (m, 2H), 4.09 (br t, J = 5.3 Hz, 2H), 3.90 (br d, J = 13.9 Hz, 1H), 3.77 − 3.65 (m, 3H), 3.59 − 3.37 (m, 3H), 2.26 − 2.14 (m, 1H), 2.14 − 1.98 (m, 1H) |
| 064 | 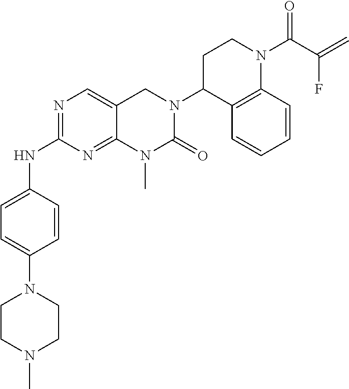 [SMILES: C=C(F)C(=O)N1CCC(N2Cc3cnc(Nc4ccc(N5CCN(C)CC5)cc4)nc3N(C)C2=O)c2ccccc21] | 557.2 | δ: 9.25 (s, 1H), 8.18 (s, 1H), 7.97 (s, 1H), 7.56 (br d, J = 8.8 Hz, 2H), 7.38 (br d, J = 8.0 Hz, 1H), 7.30 − 7.22 (m, 1H), 7.21 − 7.14 (m, 2H), 6.87 (br d, J = 8.9 Hz, 2H), 5.75 − 5.66 (m, 1H), 5.40 (br dd, J = 3.9, 17.1 Hz, 1H), 5.32 (br dd, J = 3.8, 15.7 Hz, 1H), 4.24 (br d, J = 13.9 Hz, 1H), 4.15 − 4.01 (m, 1H), 3.93 − 3.83 (m, 1H), 3.82 − 3.69 (m, 1H), 3.33 (s, 3H), 3.07 (br s, 4H), 2.50 (br s, 4H), 2.32 − 2.24 (m, 3H), 2.23 − 2.06 (m, 2H) |
| 065 | 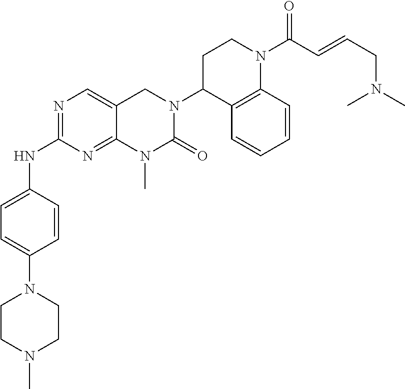 | 596.4 | δ: 9.24 (s, 1 H) 8.19 (s, 1 H) 7.98 (s, 1 H) 7.57 (d, J = 9.01 Hz, 2 H) 7.30 (br s, 4 H) 6.88 (br d, J = 8.88 Hz, 2 H) 6.75 (dt, J = 15.10, 5.96 Hz, 1 H) 6.46 (br d, J = 15.26 Hz, 1 H) 5.66 (br t, J = 8.32 Hz, 1 H) 4.10 − 4.28 (m, 2 H) 3.91 (d, J = 14.01 Hz, 1 H) 3.66 − 3.77 (m, 1 H) 3.33 (s, 3 H) 2.98 − 3.13 (m, 6 H) 2.46 − 2.50 (m, 4 H) 2.25 (s, 3 H) 2.19 (s, 7 H) 1.98 − 2.09 (m, 1 H) |
| 066 | 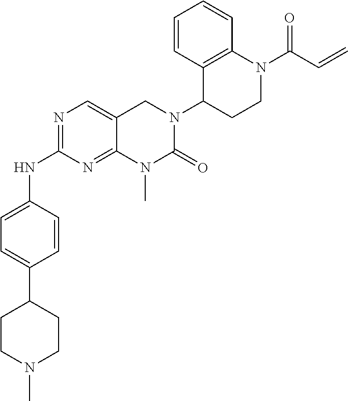 [SMILES: C=CC(=O)N1CCC(N2Cc3cnc(Nc4ccc(C5CCN(C)CC5)cc4)nc3N(C)C2=O)c2ccccc21] | 538.2 | δ: 9.42 (s, 1H), 8.23 (s, 1H), 8.01 (s, 1H), 7.65 (br d, J = 8.5 Hz, 2H), 7.27 (br dd, J = 5.3, 8.3 Hz, 2H), 7.19 (br s, 2H), 7.13 (br d, J = 8.5 Hz, 2H), 6.71 − 6.58 (m, 1H), 6.26 (dd, J = 2.0, 16.8 Hz, 1H), 5.77 (dd, J = 1.9, 10.4 Hz, 1H), 5.67 (br dd, J = 7.8, 8.8 Hz, 1H), 4.29 − 4.11 (m, 2H), 3.92 (br d, J = 14.1 Hz, 1H), 3.71 (br s, 1H), 3.34 (s, 3H), 2.94 (br d, J = 10.9 Hz, 2H), 2.47 − 2.37 (m, 1H), 2.27 (s, 3H), 2.23 − 2.15 (m, 1H), 2.14 − 2.00 (m, 3H), 1.78 − 1.59 (m, 4H) |
| 067 | 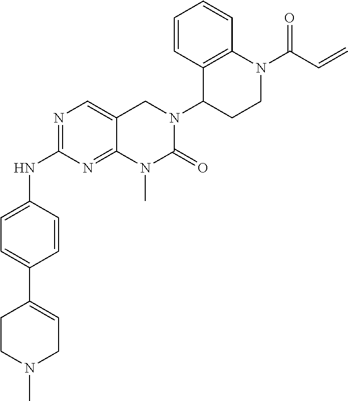 | 536.3 | δ: 9.61 − 9.49 (m, 1H), 8.09 − 8.02 (m, 1H), 7.79 − 7.67 (m, 2H), 7.43 − 7.11 (m, 6H), 6.72 − 6.59 (m, 1H), 6.31 − 6.21 (m, 1H), 6.08 (br s, 1H), 5.83 − 5.75 (m, 1H), 5.73 − 5.64 (m, 1H), 4.33 − 4.14 (m, 2H), 3.98 − 3.89 (m, 1H), 3.79 − 3.64 (m, 1H), 3.36 (br s, 3H), 3.02 − 2.95 (m, 2H), 2.57 − 2.54 (m, 2H), 2.48 − 2.42 (m, 2H), 2.32 − 2.16 (m, 4H), 2.13 − 2.00 (m, 1H) |
| 068 | 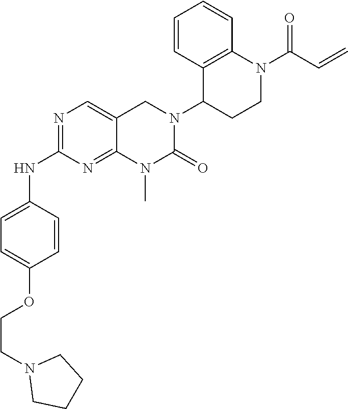 [SMILES: C=CC(=O)N1CCC(N2Cc3cnc(Nc4ccc(OCCN5CCCC5)cc4)nc3N(C)C2=O)c2ccccc21] | 554.2 | δ: 9.33 (s, 1H), 8.22 (s, 1H), 8.01 − 7.96 (m, 1H), 7.61 (br d, J = 8.6 Hz, 2H), 7.33 − 7.11 (m, 4H), 6.88 (br d, J = 8.7 Hz, 2H), 6.64 (br dd, J = 10.7, 16.9 Hz, 1H), 6.26 (br d, J = 17.1 Hz, 1H), 5.77 (br d, J = 11.4 Hz, 1H), 5.67 (br t, J = 8.2 Hz, 1H), 4.25 − 4.15 (m, 2H), 4.13 − 3.96 (m, 3H), 3.90 (br d, J = 14.2 Hz, 1H), 3.71 (br t, J = 10.3 Hz, 1H), 2.91 (br t, J = 5.1 Hz, 2H), 2.67 (br s, 5H), 2.19 (br d, J = 3.7 Hz, 1H), 2.10 − 2.02 (m, 1H), 1.73 (br s, 5H) |
| 069 | 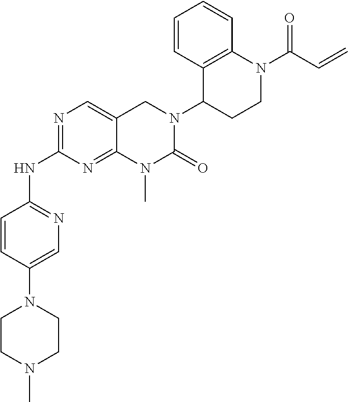 [SMILES: C=CC(=O)N1CCC(N2Cc3cnc(Nc4ccc(N5CCN(C)CC5)cn4)nc3N(C)C2=O)c2ccccc21] | 540.1 | δ: 9.32 (s, 1 H) 8.29 − 8.35 (m, 1 H) 7.94 − 8.08 (m, 3 H) 7.41 (dd, J = 9.19, 2.94 Hz, 1 H) 7.14 − 7.31 (m, 4 H) 6.65 (br dd, J = 16.82, 10.32 Hz, 1 H) 6.26 (dd, J = 16.82, 1.94 Hz, 1 H) 5.63 − 5.81 (m, 2 H) 4.22 − 4.30 (m, 1 H) 4.18 (dt, J = 12.88, 4.44 Hz, 1 H) 3.90 − 3.97 (m, 1 H) 3.66 − 3.76 (m, 1 H) 3.26 (br s, 3 H) 3.05 − 3.16 (m, 4 H) 2.43 − 2.48 (m, 4 H) 2.22 (s, 3 H) 2.15 − 2.20 (m, 1 H) 1.99 − 2.12 (m, 1 H) |
| 070 | 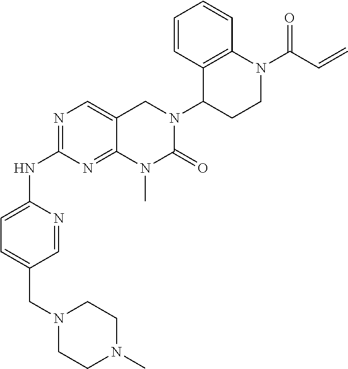 [SMILES: C=CC(=O)N1CCC(N2Cc3cnc(Nc4ccc(CN5CCN(C)CC5)cn4)nc3N(C)C2=O)c2ccccc21] | 554.2 | δ: 2.02 − 2.11 (m, 1 H) 2.14 (s, 3 H) 2.17 − 2.23 (m, 2 H) 2.26 − 2.44 (m, 7 H) 3.35 (s, 3 H) 3.41 (s, 2 H) 3.67 − 3.76 (m, 1 H) 3.96 (d, J = 14.26 Hz, 1 H) 4.18 (dt, J = 12.85, 4.33 Hz, 1 H) 4.28 (d, J = 14.26 Hz, 1 H) 5.68 (br t, J = 8.25 Hz, 1 H) 5.75 − 5.81 (m, 1 H) 6.26 (dd, J = 16.76, 2.00 Hz, 1 H) 6.65 (br dd, J = 16.70, 10.32 Hz, 1 H) 7.15 − 7.22 (m, 2 H) 7.22 − 7.31 (m, 2 H) 7.65 (dd, J = 8.57, 2.06 Hz, 1 H) 8.09 (s, 1 H) 8.14 (d, J = 1.63 Hz, 1 H) 8.19 (d, J = 8.51 Hz, 1 H) 9.60 (s, 1 H) |
| 071 | 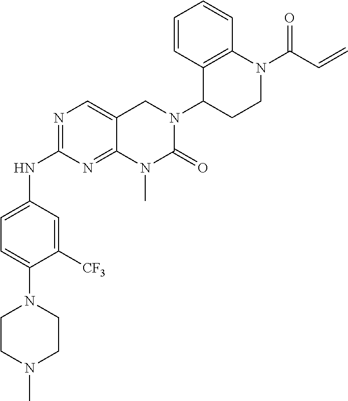 [SMILES: C=CC(=O)N1CCC(N2Cc3cnc(Nc4ccc(N5CCN(C)CC5)c(C(F)(F)F)c4)nc3N(C)C2=O)c2ccccc21] | 607.4 | δ: 9.76 (s, 1 H) 8.27 (d, J = 2.25 Hz, 1 H) 8.06 (s, 1 H) 7.88 (dd, J = 8.69, 2.06 Hz, 1 H) 7.50 (d, J = 8.88 Hz, 1 H) 7.22 − 7.34 (m, 2 H) 7.13 − 7.21 (m, 2 H) 6.65 (br dd, J = 17.07, 10.19 Hz, 1 H) 6.26 (dd, J = 16.76, 2.00 Hz, 1 H) 5.75 − 5.80 (m, 1 H) 5.67 (t, J = 8.44 Hz, 1 H) 4.26 (d, J = 14.26 Hz, 1 H) 4.18 (dt, J = 13.10, 4.64 Hz, 1 H) 3.94 (d, J = 14.26 Hz, 1 H) 3.66 − 3.77 (m, 1 H) 3.35 (s, 3 H) 2.81 (br d, J = 4.38 Hz, 4 H) 2.35 − 2.47 (m, 4 H) 2.21 (s, 3 H) 2.18 (br s, 1 H) 2.01 − 2.12 (m, 1 H) |
| 072 | 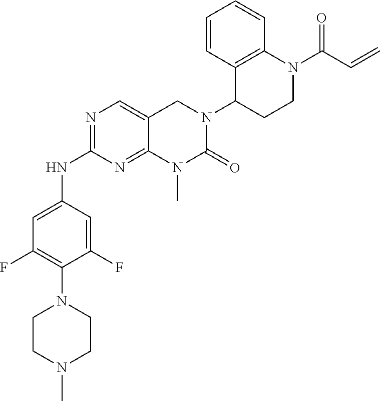 | 575.2 | δ: 9.78 (s, 1H), 8.08 (s, 1H), 7.47 (br d, J = 12.3 Hz, 2H), 7.19 (s, 3H), 6.71 − 6.59 (m, 1H), 6.30 − 6.21 (m, 1H), 5.81 − 5.74 (m, 1H), 5.67 (br t, J = 8.3 Hz, 1H), 4.27 (br d, J = 14.3 Hz, 1H), 4.22 − 4.13 (m, 1H), 3.98 − 3.90 (m, 1H), 3.76 − 3.66 (m, 1H), 3.03 (br s, 4H), 2.40 (br s, 4H), 2.21 (s, 4H), 2.12 − 1.98 (m, 1H) |
| 073 | 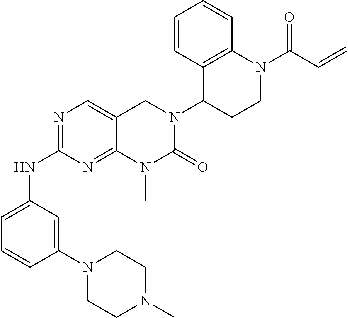 [SMILES: C=CC(=O)N1CCC(N2Cc3cnc(Nc4cccc(N5CCN(C)CC5)c4)nc3N(C)C2=O)c2ccccc21] | 539.2 | δ: 1.97 − 2.15 (m, 1 H) 2.22 (s, 3 H) 2.42 − 2.47 (m, 3 H) 3.08 − 3.13 (m, 3 H) 3.35 (br s, 7 H) 3.64 − 3.79 (m, 1 H) 3.92 (d, J = 14.01 Hz, 1 H) 4.06 − 4.34 (m, 2 H) 5.61 − 5.72 (m, 1 H) 5.75 − 5.79 (m, 1 H) 6.26 (dd, J = 16.82, 1.94 Hz, 1 H) 6.53 (br d, J = 8.00 Hz, 1 H) 6.65 (br dd, J = 16.70, 10.32 Hz, 1 H) 7.02 − 7.36 (m, 5 H) 7.45 (s, 1 H) 8.02 (s, 1 H) 9.32 (s, 1 H) |
| 074 | 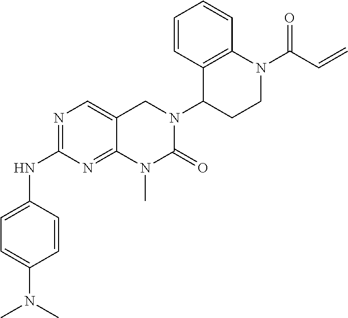 [SMILES: C=CC(=O)N1CCC(N2Cc3cnc(Nc4ccc(N(C)C)cc4)nc3N(C)C2=O)c2ccccc21] | 484.2 | δ: 9.13 (s, 1 H) 7.95 (s, 1 H) 7.51 (d, J = 9.01 Hz, 2 H) 7.22 − 7.33 (m, 2 H) 7.18 (d, J = 4.00 Hz, 2 H) 6.70 (d, J = 9.01 Hz, 2 H) 6.58 − 6.67 (m, 1 H) 6.26 (dd, J = 16.82, 2.06 Hz, 1 H) 5.73 − 5.81 (m, 1 H) 5.66 (dd, J = 9.07, 7.44 Hz, 1 H) 4.12 − 4.26 (m, 2 H) 3.89 (d, J = 13.88 Hz, 1 H) 3.66 − 3.77 (m, 1 H) 3.35 (br s, 3 H) 2.83 (s, 6 H) 2.20 (qd, J = 8.07, 3.81 Hz, 1 H) 1.99 − 2.12 (m, 1 H) |
| 075 | 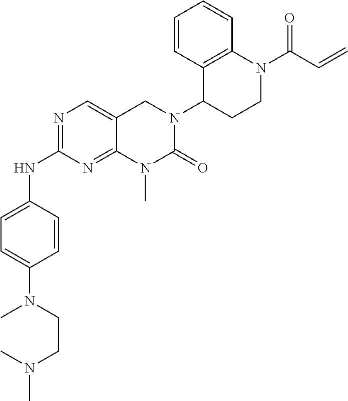 [SMILES: C=CC(=O)N1CCC(N2Cc3cnc(Nc4ccc(N(C)CCN(C)C)cc4)nc3N(C)C2=O)c2ccccc21] | 541.2 | δ: 9.16 − 9.07 (m, 1H), 8.23 − 8.17 (m, 1H), 7.99 − 7.90 (m, 1H), 7.49 (d, J = 9.0 Hz, 2H), 7.32 − 7.20 (m, 2H), 7.18 (d, J = 4.1 Hz, 2H), 6.70 − 6.57 (m, 3H), 6.26 (dd, J = 2.1, 16.8 Hz, 1H), 5.83 − 5.73 (m, 1H), 5.67 (dd, J = 7.3, 9.3 Hz, 1H), 4.25 − 4.12 (m, 2H), 3.92 − 3.86 (m, 1H), 3.77 − 3.65 (m, 1H), 3.36 (br t, J = 7.1 Hz, 2H), 3.32 (s, 3H), 2.85 (s, 3H), 2.39 (br t, J = 7.1 Hz, 2H), 2.20 (s, 7H), 2.09 − 1.99 (m, 1H) |
| 076 | 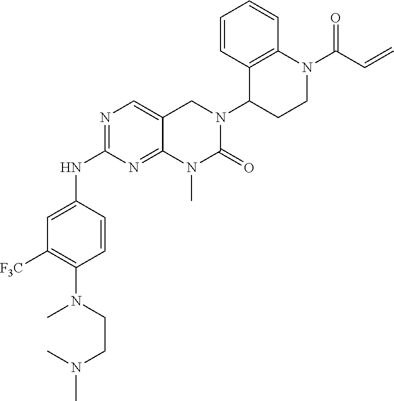 [SMILES: C=CC(=O)N1CCC(N2Cc3cnc(Nc4ccc(N(C)CCN(C)C)c(C(F)(F)F)c4)nc3N(C)C2=O)c2ccccc21] | 609.4 | δ: 9.78 (s, 1H), 8.27 (br s, 1H), 8.21 (s, 1H), 8.07 (s, 1H), 7.90 (br d, J = 8.6 Hz, 1H), 7.54 (br d, J = 8.9 Hz, 1H), 7.32 − 7.14 (m, 4H), 6.65 (br dd, J = 10.4, 16.7 Hz, 1H), 6.26 (dd, J = 1.7, 16.8 Hz, 1H), 5.82 − 5.75 (m, 1H), 5.67 (br t, J = 8.2 Hz, 1H), 4.30 − 4.15 (m, 2H), 3.99 − 3.89 (m, 1H), 3.77 − 3.66 (m, 1H), 3.35 (s, 3H), 3.00 − 2.92 (m, 2H), 2.59 (s, 3H), 2.37 − 2.29 (m, 2H), 2.15 (s, 7H), 2.12 − 2.00 (m, 1H) |
| 077 | 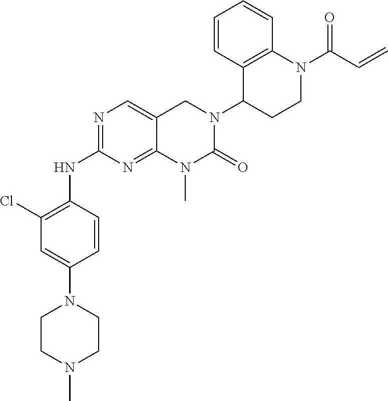 [SMILES: C=CC(=O)N1CCC(N2Cc3cnc(Nc4ccc(N5CCN(C)CC5)cc4Cl)nc3N(C)C2=O)c2ccccc21] | 573.1 | δ: 8.57 − 8.47 (m, 1H), 7.95 − 7.83 (m, 1H), 7.48 − 7.39 (m, 1H), 7.31 − 7.20 (m, 2H), 7.20 − 7.12 (m, 2H), 7.00 − 6.97 (m, 1H), 6.93 − 6.87 (m, 1H), 6.69 − 6.59 (m, 1H), 6.29 − 6.21 (m, 1H), 5.81 − 5.73 (m, 1H), 5.70 − 5.60 (m, 1H), 4.24 − 4.13 (m, 2H), 3.92 − 3.83 (m, 1H), 3.75 − 3.64 (m, 1H), 3.23 (s, 3H), 3.16 − 3.09 (m, 4H), 2.47 − 2.40 (m, 4H), 2.21 (s, 3H), 2.19 − 2.13 (m, 1H), 2.10 − 1.98 (m, 1H) |
| 078 | 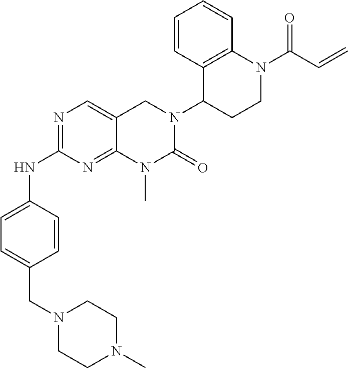 [SMILES: C=CC(=O)N1CCC(N2Cc3cnc(Nc4ccc(CN5CCN(C)CC5)cc4)nc3N(C)C2=O)c2ccccc21] | 553.3 | δ: 9.47 (s, 1 H) 8.18 (s, 1 H) 8.02 (s, 1 H) 7.68 (d, J = 8.25 Hz, 2 H) 7.22 − 7.32 (m, 2 H) 7.11 − 7.21 (m, 4 H) 6.65 (br dd, J = 16.82, 10.44 Hz, 1 H) 6.26 (br d, J = 16.76 Hz, 1 H) 5.77 (br d, J = 11.51 Hz, 1 H) 5.67 (br t, J = 8.38 Hz, 1 H) 4.25 (d, J = 14.13 Hz, 1 H) 4.18 (dt, J = 12.69, 4.10 Hz, 1 H) 3.92 (d, J = 14.13 Hz, 1 H) 3.65 − 3.77 (m, 1 H) 3.37 (s, 2 H) 3.35 (s, 3 H) 2.34 (br s, 7 H) 2.17 − 2.26 (m, 2 H) 2.16 (s, 3 H) 1.98 − 2.10 (m, 1 H) |
| 079 | 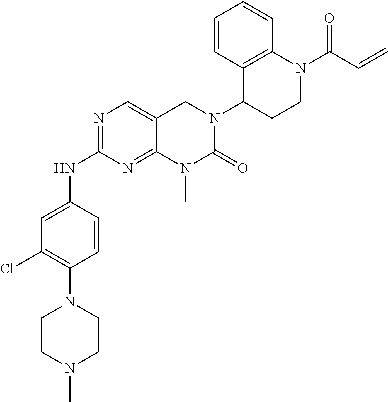 [SMILES: C=CC(=O)N1CCC(N2Cc3cnc(Nc4ccc(N5CCN(C)CC5)c(Cl)c4)nc3N(C)C2=O)c2ccccc21] | 573.1 | δ: 9.55 (s, 1H), 8.04 (s, 1H), 7.92 (d, J = 2.5 Hz, 1H), 7.61 (dd, J = 2.4, 8.8 Hz, 1H), 7.32 − 7.15 (m, 4H), 7.10 (d, J = 8.8 Hz, 1H), 6.71 − 6.60 (m, 1H), 6.26 (dd, J = 2.1, 16.8 Hz, 1H), 5.82 − 5.74 (m, 1H), 5.67 (dd, J = 7.3, 9.4 Hz, 1H), 4.25 (d, J = 13.9 Hz, 1H), 4.18 (td, J = 4.4, 12.9 Hz, 1H), 3.93 (d, J = 14.3 Hz, 1H), 3.76 − 3.67 (m, 1H), 3.34 (s, 3H), 2.91 (br s, 4H), 2.49 − 2.39 (m, 4H), 2.27 − 2.15 (m, 4H), 2.12 − 1.99 (m, 1H). |
| 080 | 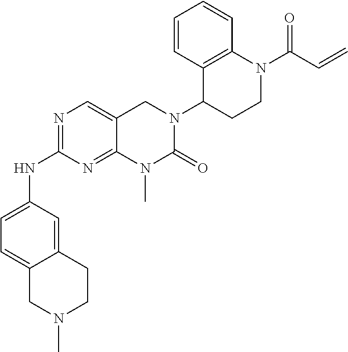 | 510.2 | δ: 9.37 (s, 1 H) 8.02 (s, 1 H) 7.52 (s, 1 H) 7.45 (dd, J = 8.34, 2.03 Hz, 1 H) 7.22 − 7.34 (m, 2 H) 7.15 − 7.21 (m, 2 H) 6.93 (d, J = 8.34 Hz, 1 H) 6.65 (dd, J = 16.75, 10.31 Hz, 1 H) 6.26 (dd, J = 16.81, 2.15 Hz, 1 H) 5.77 (dd, J = 10.37, 2.03 Hz, 1 H) 5.67 (dd, J = 9.42, 7.51 Hz, 1 H) 4.13 − 4.28 (m, 2 H) 3.92 (d, J = 14.19 Hz, 1 H) 3.68 − 3.75 (m, 1 H) 3.43 (s, 2 H) 3.34 (s, 3 H) 2.74 − 2.83 (m, 2 H) 2.59 (br t, J = 5.54 Hz, 2 H) 2.34 (s, 3 H) 2.16 − 2.25 (m, 1 H) 1.99 − 2.12 (m, 1 H) |
| 081 | 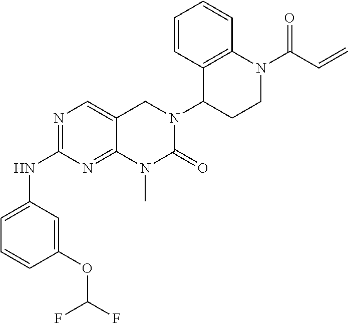 [SMILES: C=CC(=O)N1CCC(N2Cc3cnc(Nc4cccc(OC(F)F)c4)nc3N(C)C2=O)c2ccccc21] | 507.2 | δ: 9.79 − 9.69 (m, 1H), 8.16 − 8.14 (m, 1H), 8.13 − 8.04 (m, 1H), 7.87 − 7.75 (m, 1H), 7.52 (br d, J = 8.3 Hz, 1H), 7.38 − 6.99 (m, 6H), 6.73 (dd, J = 1.8, 8.1 Hz, 1H), 6.70 − 6.60 (m, 1H), 6.31 − 6.21 (m, 1H), 5.82 − 5.73 (m, 1H), 5.72 − 5.63 (m, 1H), 4.32 − 4.23 (m, 1H), 4.18 (td, J = 4.5, 12.9 Hz, 1H), 3.99 − 3.90 (m, 1H), 3.77 − 3.66 (m, 1H), 3.36 (s, 3H), 2.27 − 2.15 (m, 1H), 2.12 − 2.00 (m, 1H) |
| 082 | 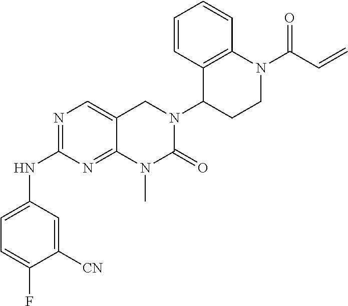 [SMILES: C=CC(=O)N1CCC(N2Cc3cnc(Nc4ccc(F)c(C#N)c4)nc3N(C)C2=O)c2ccccc21] | 484.2 | δ: 9.96 − 9.84 (m, 1H), 8.32 − 8.23 (m, 1H), 8.12 − 8.06 (m, 1H), 8.04 − 7.94 (m, 1H), 7.52 − 7.42 (m, 1H), 7.34 − 7.23 (m, 2H), 7.22 − 7.11 (m, 2H), 6.77 − 6.55 (m, 1H), 6.35 − 6.19 (m, 1H), 5.83 − 5.73 (m, 1H), 5.72 − 5.61 (m, 1H), 4.32 − 4.23 (m, 1H), 4.23 − 4.13 (m, 1H), 3.99 − 3.90 (m, 1H), 3.78 − 3.65 (m, 1H), 3.35 (s, 3H), 2.52 − 2.51 (m, 1H), 2.26 − 2.13 (m, 1H), 2.09 − 1.96 (m, 1H) |
| 083 | 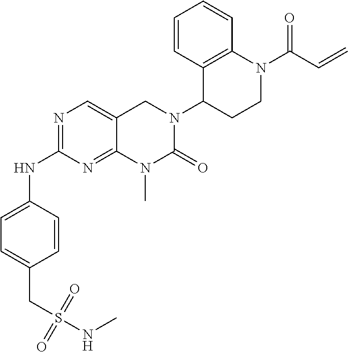 [SMILES: C=CC(=O)N1CCC(N2Cc3cnc(Nc4ccc(CS(=O)(=O)NC)cc4)nc3N(C)C2=O)c2ccccc21] | 548.1 | δ: 9.59 (s, 1H), 8.05 (s, 1H), 7.74 (d, J = 8.5 Hz, 2H), 7.34 − 7.14 (m, 6H), 6.86 (q, J = 4.9 Hz, 1H), 6.65 (br dd, J = 10.2, 17.1 Hz, 1H), 6.26 (dd, J = 2.0, 16.8 Hz, 1H), 5.82 − 5.74 (m, 1H), 5.72 − 5.64 (m, 1H), 4.38 − 4.10 (m, 4H), 3.93 (d, J = 14.0 Hz, 1H), 3.78 − 3.65 (m, 1H), 3.36 (s, 3H), 2.56 (d, J = 4.9 Hz, 3H), 2.25 − 2.14 (m, 1H), 2.12 − 1.98 (m, 1H) |
| 084 | 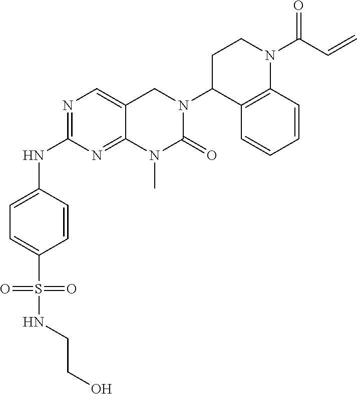 [SMILES: C=CC(=O)N1CCC(N2Cc3cnc(Nc4ccc(S(=O)(=O)NCCO)cc4)nc3N(C)C2=O)c2ccccc21] | 564.1 | δ: 8.07 (s, 1 H) 7.43 (s, 1 H) 7.21 − 7.31 (m, 2 H) 7.14 − 7.21 (m, 2 H) 6.53 − 6.70 (m, 3 H) 6.25 (dd, J = 16.76, 2.00 Hz, 1 H) 5.91 (s, 2 H) 5.77 (dd, J = 10.38, 2.00 Hz, 1 H) 5.65 (br t, J = 8.32 Hz, 1 H) 4.13 − 4.33 (m, 4 H) 3.97 (br d, J = 14.38 Hz, 1 H) 3.64 − 3.75 (m, 1 H) 3.31 (s, 2 H) 3.27 (s, 3 H) 3.05 (q, J = 5.54 Hz, 2 H) 2.12 − 2.23 (m, 1 H) 2.00 − 2.11 (m, 1 H) |
| 085 | 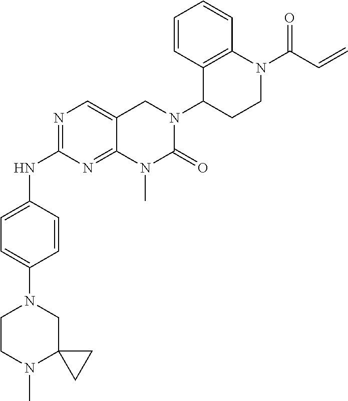 [SMILES: C=CC(=O)N1CCC(N2Cc3cnc(Nc4ccc(N5CCN(C)C6(CC6)C5)cc4)nc3N(C)C2=O)c2ccccc21] | 565.2 | δ: 9.24 (s, 1H), 8.00 − 7.94 (m, 1H), 7.55 (dd, J = 2.3, 9.0 Hz, 2H), 7.31 − 7.20 (m, 2H), 7.18 (d, J = 4.2 Hz, 2H), 6.85 (d, J = 9.0 Hz, 2H), 6.64 (br dd, J = 10.5, 16.6 Hz, 1H), 6.30 − 6.22 (m, 1H), 5.81 − 5.74 (m, 1H), 5.71 − 5.62 (m, 1H), 4.27 − 4.14 (m, 2H), 3.89 (d, J = 13.8 Hz, 1H), 3.76 − 3.66 (m, 1H), 3.32 (s, 3H), 3.06 − 3.02 (m, 2H), 2.92 − 2.88 (m, 2H), 2.86 (s, 2H), 2.27 − 2.15 (m, 4H), 2.11 − 1.99 (m, 1H), 0.67 − 0.59 (m, 2H), 0.51 − 0.42 (m, 2H) |
| 086 | 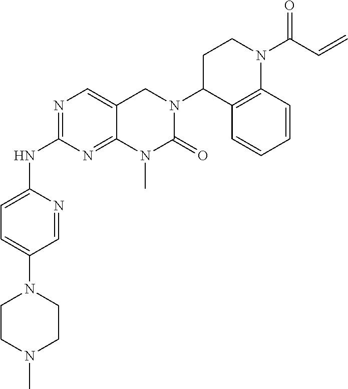 [SMILES: C=CC(=O)N1CCC(N2Cc3cnc(Nc4ccc(N5CCN(C)CC5)cn4)nc3N(C)C2=O)c2ccccc21] | 539.1 | δ: 10.73 − 11.16 (m, 1 H), 8.14 (s, 1 H), 7.99 (d, J = 2.88 Hz, 1 H), 7.93 (br s, 1 H), 7.57 −7.74 (m, 1 H), 7.34 − 7.49 (m, 1 H), 7.22 − 7.33 (m, 2 H), 7.17 (br d, J = 4.25 Hz, 2 H), 7.00 (br d, J = 5.13 Hz, 1 H), 6.66 (br dd,J = 16.82, 10.32 Hz, 1 H), 6.26 (dd, J = 16.76, 2.13 Hz, 1 H), 5.73 - 5.83 (m, 1 H), 5.64 (dd, J = 9.38, 7.25 Hz, 1 H), 4.33 (br d,J = 14.63 Hz, 1 H), 4.18 (dt, J = 12.91, 4.61 Hz, 1 H), 4.03 (br d, J = 14.51 Hz, 1 H), 3.63 − 3.86 (m, 3 H), 3.48 (br d, J = 2.63 Hz, 2H), 3.28 (s, 3 H), 3.06 − 3.24 (m, 4 H), 2.81 (s, 3 H), 2.15 − 2.26 (m, 1 H), 1.96 − 2.13 (m, 1 H) |
| 087 | 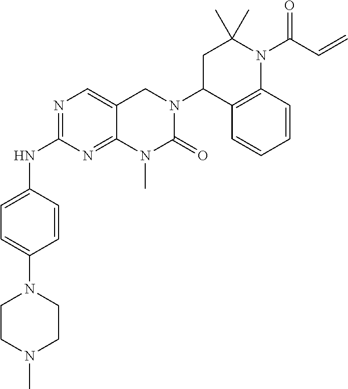 [SMILES: C=CC(=O)N1c2ccccc2C(N2Cc3cnc(Nc4ccc(N5CCN(C)CC5)cc4)nc3N(C)C2=O)CC1(C)C] | 623.2 | δ: 8.65 (s, 1H), 7.91 (s, 1H), 7.58 − 7.48 (m, 1H), 7.32 − 7.21 (m, 2H), 7.18 (d, J = 4.1 Hz, 2H), 6.98 − 6.91 (m, 1H), 6.87 − 6.82 (m, 1H), 6.65 (dd, J = 10.3, 16.8 Hz, 1H), 6.26 (dd, J = 2.1, 16.8 Hz, 1H), 5.80 − 5.75 (m, 1H), 5.66 (dd, J = 7.3, 9.3 Hz, 1H), 4.28 − 4.13 (m, 2H), 3.93 − 3.85 (m, 1H), 3.76 − 3.65 (m, 1H), 3.24 (s, 3H), 3.18 − 3.10 (m, 4H), 2.47 − 2.41 (m, 4H), 2.26 − 2.13 (m, 4H), 2.11 − 1.97 (m, 1H) |
| 088 | 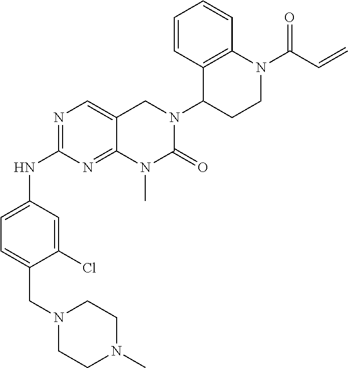 [SMILES: C=CC(=O)N1CCC(N2Cc3cnc(Nc4ccc(CN5CCN(C)CC5)c(Cl)c4)nc3N(C)C2=O)c2ccccc21] | 587.2 | δ: 9.71 (s, 1 H), 8.20 (br s, 1 H), 8.07 (s, 1 H), 7.98 (d, J = 1.96 Hz, 1 H), 7.61 (dd, J = 8.50,1.90 Hz, 1 H), 7.32 (d, J = 8.44 Hz, 1 H), 7.28 (br dd, J = 6.11, 1.96 Hz, 1 H), 7.17 − 7.22 (m, 2 H), 6.65 (br dd, J = 16.99, 10.76 Hz,1 H), 6.26 (dd, J = 16.75, 2.08 Hz, 1 H), 5.78 (dd, J = 10.33, 2.02 Hz, 1 H), 5.67 (br t, J = 8.19 Hz, 1 H), 4.26 (d, J = 14.18 Hz, 1 H),4.13 − 4.22 (m, 1 H), 3.94 (d, J = 14.06 Hz, 1 H), 3.65 − 3.76 (m, 1 H), 3.47 (s, 2 H), 3.35 (s, 3 H), 2.13 − 2.48 (m, 12 H), 1.98 −2. 12 (m, 1 H) |
| 089 | 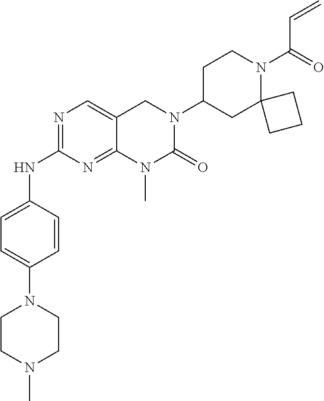 [SMILES: C=CC(=O)N1CCC(N2Cc3cnc(Nc4ccc(N5CCN(C)CC5)cc4)nc3N(C)C2=O)CC12CCC2] | 531.3 | δ: 9.22 (s, 1 H), 8.19 (s, 1 H), 8.06 (s, 1 H), 7.55 (d, J = 8.92 Hz, 2 H), 6.86 (d, J = 9.05 Hz, 2H), 6.67 (br dd, J = 3.73, 1.77 Hz, 1 H), 6.09 (dd, J = 16.75, 2.20 Hz, 1 H), 5.64 (dd, J = 10.39, 2.32 Hz, 1 H), 4.47 − 4.67 (m, 1 H),4.17 (s, 2 H), 3.97 (br dd, J = 15.71, 1.53 Hz, 1 H), 3.25 (s, 3 H), 2.96 − 3.11 (m, 4 H), 2.89 (br s, 1 H), 2.54 (br d, J = 3.55 Hz, 5H), 2.25 (s, 3 H), 2.15 (br d, J = 8.44 Hz, 2 H), 1.83 − 2.02 (m, 3 H), 1.51 − 1.79 (m, 3 H), 1.41 (br d, J = 9.17 Hz, 1 H) |
| 090 | 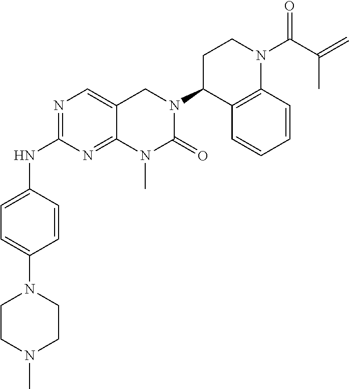 [SMILES: C=C(C)C(=O)N1CC[C@H](N2Cc3cnc(Nc4ccc(N5CCN(C)CC5)cc4)nc3N(C)C2=O)c2ccccc21] | 553.2 | δ: 9.25 (s, 1H), 8.17 (s, 1H), 8.00 (s, 1H), 7.62 − 7.52 (m, 2H), 7.37 (d, J = 8.1 Hz, 1H), 7.25 − 7.07 (m, 3H), 6.88 (d, J = 9.1 Hz, 2H), 5.71 (t, J = 8.4 Hz, 1H), 5.27 (s, 1H), 5.14 (s, 1H), 4.26 (d, J = 14.1 Hz, 1H), 4.12 (td, J = 4.4, 13.0 Hz, 1H), 3.89 (d, J = 14.1 Hz, 1H), 3.76 − 3.64 (m, 1H), 3.34 (s, 3H), 3.11 − 2.99 (m, 4H), 2.49 − 2.41 (m, 4H), 2.23 (s, 3H), 2.20 − 2.03 (m, 2H), 1.93 (s, 3H) |
| 091 | 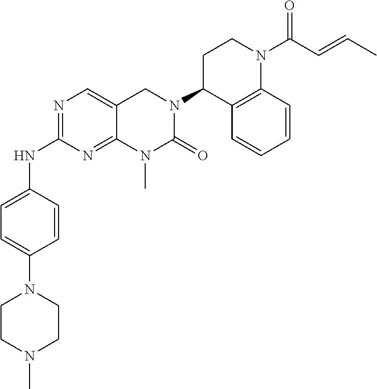 [SMILES: C/C=C/C(=O)N1CC[C@H](N2Cc3cnc(Nc4ccc(N5CCN(C)CC5)cc4)nc3N(C)C2=O)c2ccccc21] | 553.3 | δ: 9.24 (s, 1H), 8.17 (s, 1H), 7.98 (s, 1H), 7.56 (d, J = 9.1 Hz, 2H), 7.33 − 7.22 (m, 1H), 7.20 − 7.10 (m, 2H), 6.88 (d, J = 9.1 Hz, 2H), 6.85 − 6.79 (m, 1H), 6.41 − 6.33 (m, 1H), 6.14 − 6.10 (m, 1H), 6.02 − 5.90 (m, 1H), 5.72 − 5.61 (m, 1H), 5.19 − 5.09 (m, 1H), 4.27 − 4.00 (m, 2H), 3.87 (d, J = 13.6 Hz, 1H), 3.75 − 3.62 (m, 1H), 3.39 (br d, J = 6.5 Hz, 2H), 3.33 (s, 3H), 3.08 − 3.03 (m, 4H), 2.48 − 2.44 (m, 4H), 2.25 − 2.14 (m, 4H), 2.10 − 1.98 (m, 1H), 1.86 (dd, J = 1.3, 6.9 Hz, 1H) |
| 092 | 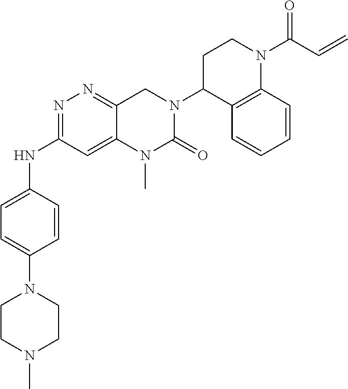 [SMILES: C=CC(=O)N1CCC(N2Cc3nnc(Nc4ccc(N5CCN(C)CC5)cc4)cc3N(C)C2=O)c2ccccc21] | 539.3 | δ: 8.38 (s, 1H), 8.17 (s, 1H), 7.60 (d, J = 8.9 Hz, 2H), 7.35 − 7.27 (m, 2H), 7.20 − 7.15 (m, 1H), 6.96 (d, J = 7.6 Hz, 1H), 6.86 (d, J = 8.9 Hz, 2H), 6.72 − 6.63 (m, 2H), 6.62 − 6.60 (m, 1H), 6.27 (dd, J = 1.9, 16.8 Hz, 1H), 5.82 − 5.76 (m, 1H), 5.53 − 5.47 (m, 1H), 5.11 (s, 1H), 4.03 − 3.83 (m, 2H), 3.31 (br d, J = 7.9 Hz, 2H), 3.07 − 3.02 (m, 4H), 2.74 (d, J = 4.6 Hz, 3H), 2.48 − 2.44 (m, 4H), 2.37 − 2.32 (m, 1H), 2.22 (s, 3H), 2.17 − 2.07 (m, 1H) |
| 093 | 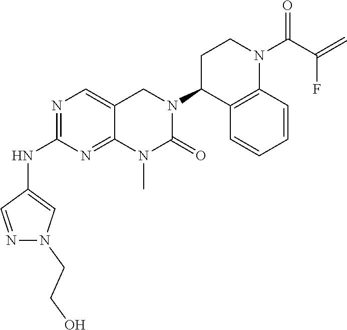 [SMILES: C=C(F)C(=O)N1CC[C@H](N2Cc3cnc(Nc4cnn(CCO)c4)nc3N(C)C2=O)c2ccccc21] | 493.1 | δ: 9.35 (br s, 1H), 7.97 (s, 1H), 7.88 (s, 1H), 7.51 (s, 1H), 7.38 (br d, J = 8.0 Hz, 1H), 7.26 (br d, J = 3.1 Hz, 1H), 7.21 − 7.14 (m, 2H), 5.74 − 5.66 (m, 1H), 5.45 − 5.28 (m, 2H), 4.86 (br s, 1H), 4.24 (d, J = 14.1 Hz, 1H), 4.09 (br t, J = 5.6 Hz, 3H), 3.88 (d, J = 14.0 Hz, 1H), 3.81 − 3.75 (m, 1H), 3.70 (br d, J = 5.1 Hz, 2H), 3.35 (br s, 3H), 2.27 − 2.08 (m, 2H) |
| 094 | 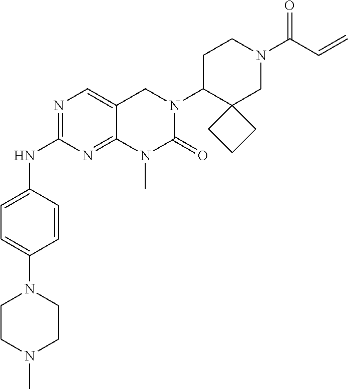 [SMILES: C=CC(=O)N1CCC(N2Cc3cnc(Nc4ccc(N5CCN(C)CC5)cc4)nc3N(C)C2=O)C2(CCC2)C1] | 531.3 | δ: 9.24 (s, 1 H) 8.20 (s, 1 H) 8.08 (s, 1 H) 7.57 (d, J = 9.13 Hz, 2 H) 6.78 − 7.03 (m, 3 H) 6.05 − 6.22 (m, 1 H) 5.63 − 5.78 (m, 1 H) 4.48 − 4.76 (m, 1 H) 4.43 (br d, J = 14.01 Hz, 1 H) 4.07 − 4.33 (m, 3 H) 3.28 (s, 3 H) 2.98 − 3.21 (m, 5 H) 2.55 − 2.82 (m, 1 H) 2.47 (br d, J = 4.88 Hz, 4 H) 2.24 (s, 3 H) 1.36 − 1.97 (m, 8 H) |
| 095 | 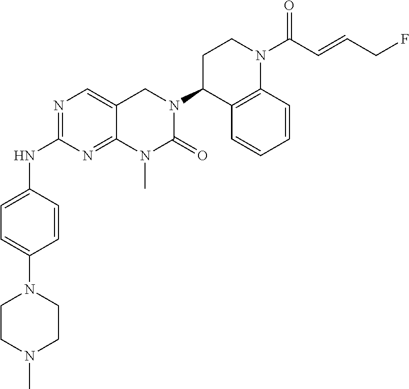 [SMILES: CN1CCN(c2ccc(Nc3ncc4c(n3)N(C)C(=O)N([C@H]3CCN(C(=O)/C=C/CF)c5ccccc53)C4)cc2)CC1] | 571.2 | δ: 9.23 (s, 1H), 8.15 (s, 1H), 8.01 − 7.93 (m, 1H), 7.56 (d, J = 9.1 Hz, 2H), 7.34 − 7.13 (m, 4H), 6.95 − 6.80 (m, 3H), 6.61 − 6.47 (m, 1H), 5.67 (dd, J = 7.4, 9.3 Hz, 1H), 5.21 (dd, J = 1.8, 3.9 Hz, 1H), 5.10 (dd, J = 1.8, 3.9 Hz, 1H), 4.28 − 4.13 (m, 2H), 3.95 − 3.86 (m, 1H), 3.77 − 3.66 (m, 1H), 3.32 (s, 3H), 3.09 − 3.01 (m, 4H), 2.48 − 2.41 (m, 4H), 2.29 − 2.15 (m, 4H), 2.07 (ddd, J = 4.3, 9.7, 13.7 Hz, 1H) |
| 096 | 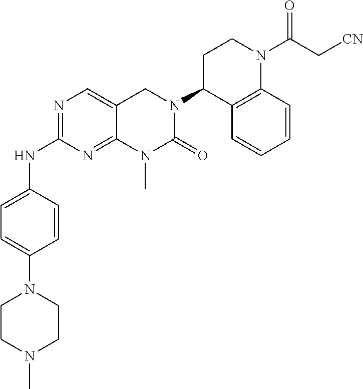 [SMILES: CN1CCN(c2ccc(Nc3ncc4c(n3)N(C)C(=O)N([C@H]3CCN(C(=O)CC#N)c5ccccc53)C4)cc2)CC1] | 552.2 | δ: 9.27 (s, 1H), 8.13 (s, 1H), 7.97 (s, 1H), 7.58 (br d, J = 8.9 Hz, 2H), 7.31 − 7.24 (m, 1H), 7.18 (br s, 2H), 6.90 (br d, J = 8.8 Hz, 2H), 5.79 − 5.59 (m, 1H), 4.34 − 4.15 (m, 3H), 4.03 − 3.82 (m, 2H), 3.76 − 3.60 (m, 2H), 3.33 (s, 3H), 3.16 (br s, 4H), 2.89 − 2.72 (m, 4H), 2.48 − 2.46 (m, 3H), 2.19 − 2.03 (m, 2H), 1.47 (br d, J = 9.4 Hz, 1H), 0.97 − 0.89 (m, 1H) |
| 097 | 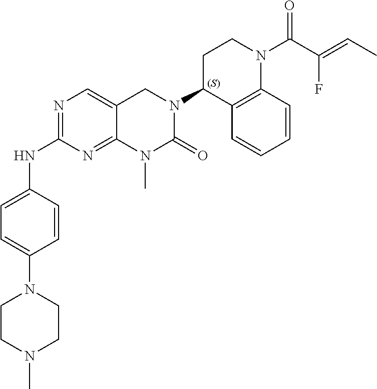 | 571.3 | δ: 9.27 (s, 1H), 7.97 (s, 1H), 7.56 (d, J = 9.0 Hz, 2H), 7.51 − 7.40 (m, 1H), 7.30 − 7.22 (m, 1H), 7.21 − 7.11 (m, 2H), 6.87 (d, J = 9.0 Hz, 2H), 5.77 − 5.66 (m, 2H), 4.22 (d, J = 13.8 Hz, 1H), 4.10 − 4.01 (m, 1H), 3.91 − 3.69 (m, 2H), 3.33 (s, 3H), 3.09 − 3.02 (m, 4H), 2.47 − 2.41 (m, 4H), 2.21 (s, 4H), 2.11 (br s, 1H), 1.73 − 1.58 (m, 3H) |
| 098 | 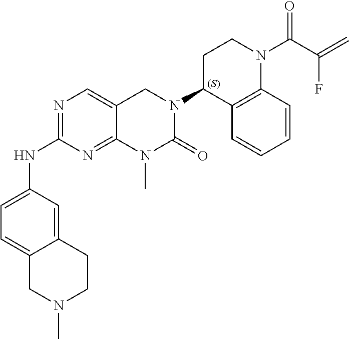 | 528.2 | δ: 9.41 (s, 1 H), 8.15 (s, 1 H), 8.02 (s, 1 H), 7.55 (s, 1 H), 7.48 (br d, J = 8.38 Hz, 1 H), 7.39 (br d, J = 8.00 Hz, 1 H), 7.22 − 7.30 (m, 1 H), 7.15 − 7.21 (m, 2 H), 6.96 (br d, J = 8.25 Hz, 1 H), 5.71 (br t, J = 8.38 Hz, 1 H), 5.27 − 5.47 (m, 2 H), 4.26 (br d, J = 14.01 Hz, 1 H), 4.04 − 4.14 (m, 1 H), 3.90 (br d, J = 14.01 Hz, 1 H), 3.71 − 3.81 (m, 1 H), 3.57 (br s, 2 H), 3.34 (s, 3 H), 2.68 − 2.88 (m, 4 H), 2.43 (br s, 3 H), 2.10 − 2.26 (m, 2 H) |
| 099 | 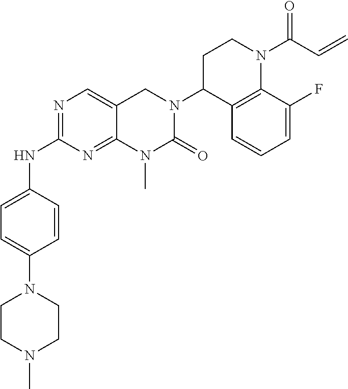 [SMILES: C=CC(=O)N1CCC(N2Cc3cnc(Nc4ccc(N5CCN(C)CC5)cc4)nc3N(C)C2=O)c2cccc(F)c21] | 557.3 | δ: 9.31 (s, 1 H), 8.00 (s, 1 H), 7.60 (br d, J = 8.88 Hz, 2 H), 7.19 − 7.27 (m, 2 H), 7.06 (br d, J = 7.13 Hz, 1 H), 6.93 (br d, J = 8.75 Hz, 2 H), 6.42 − 6.61 (m, 1 H), 6.25 (br d, J = 15.38 Hz, 1 H), 5.77 (br d, J = 11.13 Hz, 1 H), 5.66 (br d, J = 3.50 Hz, 1 H), 4.06 − 4.40 (m, 2 H), 3.78 − 4.02 (m, 2 H), 3.31 (br s, 3 H), 3.11 (br s, 5 H), 2.67 (br s, 4 H), 1.78 − 2.37 (m, 4 H) |
| 100 | 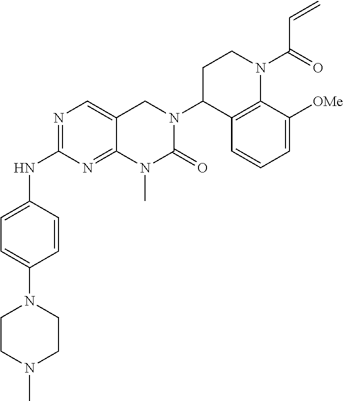 [SMILES: C=CC(=O)N1CCC(N2Cc3cnc(Nc4ccc(N5CCN(C)CC5)cc4)nc3N(C)C2=O)c2cccc(OC)c21] | 569.2 | δ: 9.16 − 9.34 (m, 1 H) 8.16 − 8.28 (m, 1 H) 7.86 − 8.06 (m, 1 H) 7.55 (br s, 2 H) 7.15 − 7.29 (m, 1 H) 6.96 − 7.09 (m, 1 H) 6.72 − 6.93 (m, 3 H) 6.07 − 6.38 (m, 2 H) 5.41 − 5.77 (m, 2 H) 3.79 − 4.70 (m, 4 H) 3.73 (s, 3 H) 3.34 − 3.41 (m, 3 H) 3.01 − 3.09 (m, 4 H) 2.40 − 2.47 (m, 4 H) 1.80 − 2.27 (m, 5 H) |
| 101 | 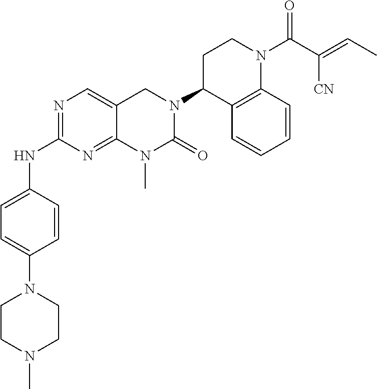 [SMILES: C/C=C(\C#N)C(=O)N1CC[C@H](N2Cc3cnc(Nc4ccc(N5CCN(C)CC5)cc4)nc3N(C)C2=O)c2ccccc21] | 578.2 | δ: 9.27 (s, 1H), 8.14 (s, 1H), 7.88 (s, 1H), 7.57 (d, J = 9.0 Hz, 2H), 7.39 (q, J = 7.1 Hz, 1H), 7.31 − 7.18 (m, 4H), 6.94 − 6.82 (m, 2H), 5.74 (dd, J = 7.9, 9.1 Hz, 1H), 4.25 (d, J = 14.1 Hz, 1H), 4.16 (td, J = 4.3, 12.9 Hz, 1H), 3.91 (d, J = 14.1 Hz, 1H), 3.77 − 3.66 (m, 1H), 3.33 (s, 3H), 3.09 (br s, 4H), 2.58 (br s, 4H), 2.32 (br d, J = 3.8 Hz, 3H), 2.25 − 2.19 (m, 1H), 2.18 − 2.04 (m, 4H) |
| 102 | 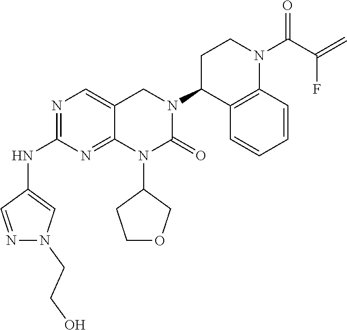 [SMILES: C=C(F)C(=O)N1CC[C@H](N2Cc3cnc(Nc4cnn(CCO)c4)nc3N(C3CCOC3)C2=O)c2ccccc21] | 549.1 | δ: 9.33 (br d, J = 19.1 Hz, 1H), 8.18 (br s, 0.1H), 8.02 (br s, 1H), 7.87 (br s, 1H), 7.50 (br s, 1H), 7.39 (br d, J = 7.6 Hz, 1H), 7.31 − 7.11 (m, 3H), 5.67 (br s, 1H), 5.50 − 5.27 (m, 3H), 4.87 (br s, 1H), 4.22 − 3.64 (m, 12H), 2.43 − 2.29 (m, 1H), 2.26 − 2.02 (m, 3H). |
| 103 | 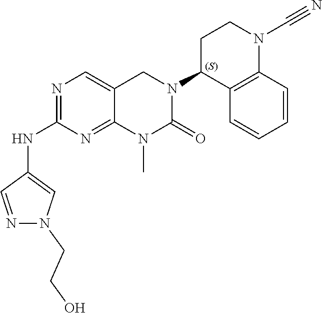 | 446.1 | δ: 9.37 (br s, 1H), 8.01 − 7.84 (m, 2H), 7.51 (br s, 1H), 7.34 (br t, J = 7.6 Hz, 1H), 7.16 − 7.00 (m, 3H), 5.66 (br dd, J = 5.2, 10.2 Hz, 1H), 4.89 (br s, 1H), 4.17 (br d, J = 13.9 Hz, 1H), 4.09 (br s, 2H), 4.01 − 3.89 (m, 2H), 3.85 (br d, J = 14.0 Hz, 1H), 3.70 (br s, 2H), 3.34 − 3.28 (m, 3H), 2.34 − 2.20 (m, 1H), 2.12 − 2.02 (m, 1H) |
| 104 | 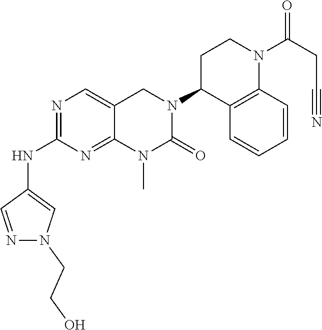 [SMILES: CN1C(=O)N([C@H]2CCN(C(=O)CC#N)c3ccccc32)Cc2cnc(Nc3cnn(CCO)c3)nc21] | 488.1 | δ: 9.38 (br s, 1H), 7.96 (s, 1H), 7.88 (s, 1H), 7.51 (s, 1H), 7.32 − 7.24 (m, 1H), 7.18 (br d, J = 2.8 Hz, 2H), 5.67 (br s, 1H), 4.32 − 4.16 (m, 3H), 4.09 (br t, J = 5.4 Hz, 2H), 3.99 − 3.82 (m, 2H), 3.74 − 3.65 (m, 3H), 3.35 (br s, 3H), 2.23 − 2.04 (m, 2H) |
| 105 | 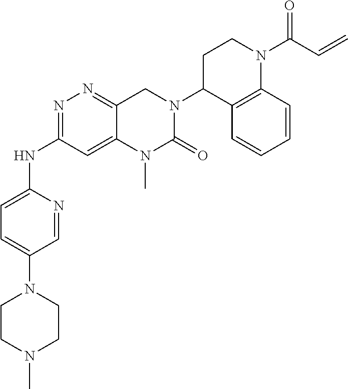 [SMILES: C=CC(=O)N1CCC(N2Cc3nnc(Nc4ccc(N5CCN(C)CC5)cn4)cc3N(C)C2=O)c2ccccc21] | 540.3 | δ: 9.02 (s, 1H), 8.28 (d, J = 9.0 Hz, 1H), 7.98 (d, J = 2.9 Hz, 1H), 7.46 (dd, J = 3.0, 9.3 Hz, 1H), 7.39 (br d, J = 4.6 Hz, 1H), 7.28 − 7.20 (m, 1H), 7.03 (d, J = 7.6 Hz, 1H), 6.82 (q, J = 4.5 Hz, 1H), 6.71 − 6.68 (m, 1H), 6.40 − 6.27 (m, 1H), 5.93 − 5.78 (m, 1H), 5.65 − 5.49 (m, 2H), 4.09 − 4.00 (m, 1H), 4.00 − 3.91 (m, 1H), 3.38 − 3.37 (m, 2H), 3.19 − 3.10 (m, 4H), 2.83 − 2.74 (m, 3H), 2.54 − 2.49 (m, 4H), 2.46 − 2.35 (m, 1H), 2.28 (s, 3H), 2.25 − 2.14 (m, 1H) |
| 106 | 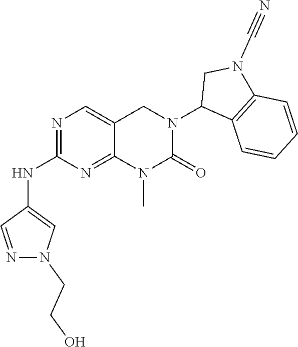 [SMILES: CN1C(=O)N(C2CN(C#N)c3ccccc32)Cc2cnc(Nc3cnn(CCO)c3)nc21] | 432.1 | δ: 9.33 (br s, 1 H) 7.83 − 8.01 (m, 2 H) 7.46 − 7.52 (m, 1 H) 7.41 (t, J = 7.69 Hz, 1 H) 7.33 (d, J = 7.38 Hz, 1 H) 7.00 − 7.15 (m, 2 H) 6.18 (dd, J = 9.69, 4.44 Hz, 1 H) 4.86 (br s, 1 H) 4.13 − 4.39 (m, 3 H) 4.03 − 4.12 (m, 2 H) 3.65 − 3.76 (m, 3 H) 3.33 − 3.37 (m, 3 H) |
| 107 | 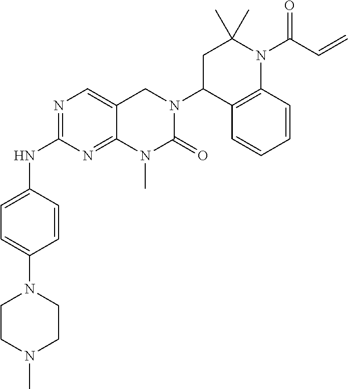 [SMILES: C=CC(=O)N1c2ccccc2C(N2Cc3cnc(Nc4ccc(N5CCN(C)CC5)cc4)nc3N(C)C2=O)CC1(C)C] | 567.2 | δ: 8.55 − 8.44 (m, 1H), 8.33 (br s, 1H), 7.85 − 7.77 (m, 1H), 7.24 − 7.11 (m, 2H), 7.10 − 7.01 (m, 1H), 7.00 − 6.79 (m, 3H), 6.60 − 6.42 (m, 2H), 5.87 − 5.57 (m, 2H), 4.70 − 4.50 (m, 2H), 4.38 − 4.25 (m, 2H), 3.24 − 3.15 (m, 4H), 3.10 − 2.94 (m, 3H), 2.49 − 2.41 (m, 4H), 2.28 − 2.17 (m, 3H), 1.89 − 1.64 (m, 2H), 1.34 − 1.06 (m, 6H). |
| 108 | 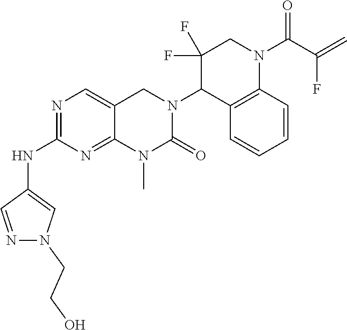 [SMILES: C=C(F)C(=O)N1CC(F)(F)C(N2Cc3cnc(Nc4cnn(CCO)c4)nc3N(C)C2=O)c2ccccc21] | 529.3 | δ: 9.42 (br s, 1 H), 8.00 (s, 1 H), 7.85 − 7.95 (m, 1 H), 7.46 − 7.59 (m, 1 H), 7.08 (t, J = 7.63 Hz, 1 H), 6.91 (d, J = 7.75 Hz, 1 H), 6.70 (d, J = 8.13 Hz, 1 H), 6.62 (t, J = 7.38 Hz, 1 H), 6.35 (br s, 1 H), 5.95 − 6.08 (m, 1 H), 5.58 − 5.77 (m, 2 H), 4.51 (br d, J = 4.50 Hz, 2 H), 4.41 (br d, J = 4.50 Hz, 2 H), 4.06 − 4.14 (m, 1 H), 3.92 − 4.01 (m, 1 H), 3.61 − 3.73 (m, 1 H), 3.43 − 3.54 (m, 1 H), 3.35 (br s, 3 H) |
| 109 | 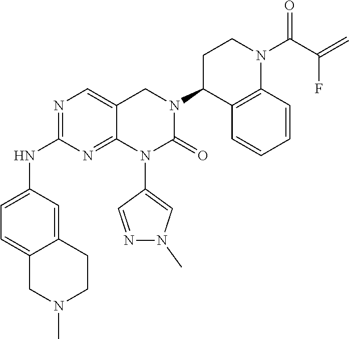 [SMILES: C=C(F)C(=O)N1CC[C@H](N2Cc3cnc(Nc4ccc5c(c4)CCN(C)C5)nc3N(c3cnn(C)c3)C2=O)c2ccccc21] | 594.2 | δ: 9.32 (s, 1H), 8.27 − 8.18 (m, 1H), 8.09 (s, 1H), 7.86 (s, 1H), 7.49 (s, 1H), 7.40 (br d, J = 8.0 Hz, 1H), 7.32 − 7.24 (m, 3H), 7.23 − 7.18 (m, 1H), 7.13 (br d, J = 8.0 Hz, 1H), 6.78 (d, J = 8.5 Hz, 1H), 5.71 (br t, J = 8.3 Hz, 1H), 5.45 − 5.27 (m, 2H), 4.37 (d, J = 14.0 Hz, 1H), 4.15 − 4.06 (m, 1H), 3.99 (br d, J = 14.0 Hz, 1H), 3.89 (s, 3H), 3.82 − 3.73 (m, 1H), 3.37 (br s, 2H), 3.17 (s, 4H), 2.65 (br t, J = 5.0 Hz, 2H), 2.55 (br t, J = 5.5 Hz, 2H), 2.35 − 2.28 (m, 3H), 2.28 − 2.12 (m, 2H) |
| 110 | 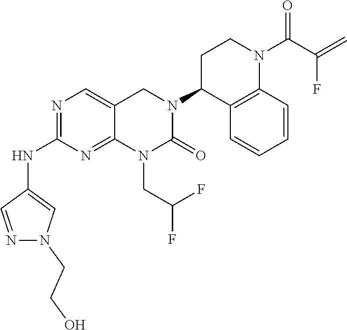 [SMILES: C=C(F)C(=O)N1CC[C@H](N2Cc3cnc(Nc4cnn(CCO)c4)nc3N(CC(F)F)C2=O)c2ccccc21] | 543.2 | δ: 9.48 − 9.36 (m, 1H), 8.05 (s, 1H), 7.92 − 7.74 (m, 1H), 7.50 (s, 1H), 7.39 (br d, J = 8.1 Hz, 1H), 7.31 − 7.23 (m, 1H), 7.21 − 7.16 (m, 2H), 6.51 − 6.17 (m, 1H), 5.72 (br t, J = 8.3 Hz, 1H), 5.48 − 5.30 (m, 2H), 4.94 − 4.84 (m, 1H), 4.56 − 4.40 (m, 2H), 4.30 (d, J = 14.1 Hz, 1H), 4.14 − 4.03 (m, 3H), 3.96 − 3.87 (m, 1H), 3.84 − 3.66 (m, 3H), 2.28 − 2.07 (m, 2H) |
| 111 | 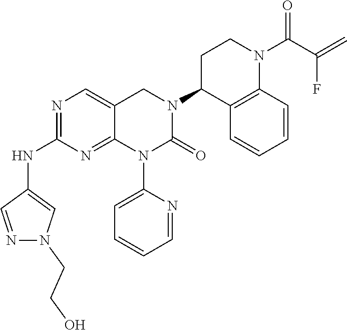 [SMILES: C=C(F)C(=O)N1CC[C@H](N2Cc3cnc(Nc4cnn(CCO)c4)nc3N(c3ccccn3)C2=O)c2ccccc21] | 556.2 | δ: ppm 9.20 − 9.52 (m, 1 H) 8.61 − 8.76 (m, 1 H) 8.00 − 8.17 (m, 2 H) 7.52 − 7.65 (m, 2 H) 7.35 − 7.44 (m, 1 H) 7.18 − 7.32 (m, 2 H) 6.90 − 7.06 (m, 1 H) 6.56 − 6.71 (m, 1 H) 5.72 (br t, J = 8.25 Hz, 1 H) 5.26 − 5.48 (m, 2 H) 4.81 (br t, J = 5.25 Hz, 1 H) 4.46 (br d, J = 14.13 Hz, 1 H) 4.08 − 4.15 (m, 1 H) 4.05 (br d, J = 14.13 Hz, 1 H) 3.73 − 3.86 (m, 3 H) 3.56 (br s, 2 H) 2.12 − 2.35 (m, 2 H) |
| 112 | 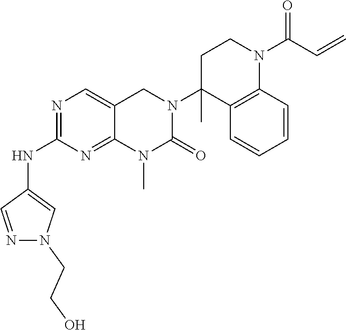 [SMILES: C=CC(=O)N1CCC(C)(N2Cc3cnc(Nc4cnn(CCO)c4)nc3N(C)C2=O)c2ccccc21] | 489.2 | δ: 9.26 (br s, 1H), 8.00 (s, 1H), 7.90 − 7.87 (m, 1H), 7.84 (s, 1H), 7.48 − 7.43 (m, 2H), 7.33 − 7.23 (m, 2H), 7.04 (br d, J = 7.6 Hz, 1H), 6.48 − 6.35 (m, 1H), 6.08 (dd, J = 2.1, 16.8 Hz, 1H), 5.67 − 5.56 (m, 1H), 4.91 − 4.79 (m, 1H), 4.19 − 4.12 (m, 1H), 4.07 (t, J = 5.5 Hz, 2H), 3.98 − 3.86 (m, 2H), 3.69 (br t, J = 5.2 Hz, 2H), 3.47 − 3.42 (m, 1H), 3.18 (br s, 3H), 3.09 − 3.01 (m, 1H), 1.76 − 1.67 (m, 4H) |
| 113 | 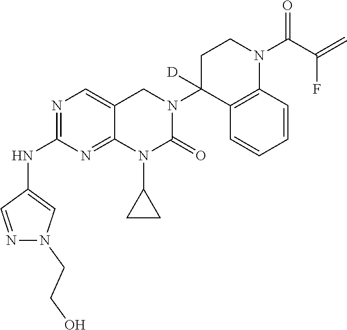 [SMILES: [2H]C1(N2Cc3cnc(Nc4cnn(CCO)c4)nc3N(C3CC3)C2=O)CCN(C(=O)C(=C)F)c2ccccc21] | 520.2 | δ = 9.48 − 9.30 (m, 1H), 8.13 − 8.00 (m, 1H), 7.96 (s, 1H), 7.53 (s, 1H), 7.39 (d, J = 8.1 Hz, 1H), 7.25 (dt, J = 1.6, 7.7 Hz, 1H), 7.17 (dt, J = 1.1, 7.5 Hz, 1H), 7.12 − 7.06 (m, 1H), 5.46 − 5.27 (m, 2H), 4.86 (t, J = 5.1 Hz, 1H), 4.11 − 4.03 (m, 4H), 3.83 − 3.63 (m, 4H), 2.89 − 2.77 (m, 1H), 2.19 (td, J = 3.9, 13.3 Hz, 1H), 2.13 − 1.99 (m, 1H), 1.10 (br s, 2H), 0.66 (br d, J = 2.0 Hz, 2H) |
| 114 | 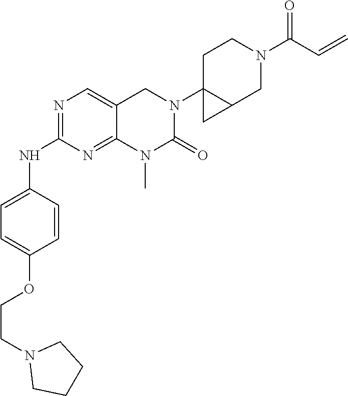 [SMILES: C=CC(=O)N1CCC2(N3Cc4cnc(Nc5ccc(OCCN6CCCC6)cc5)nc4N(C)C3=O)CC2C1] | | |
| 115 | 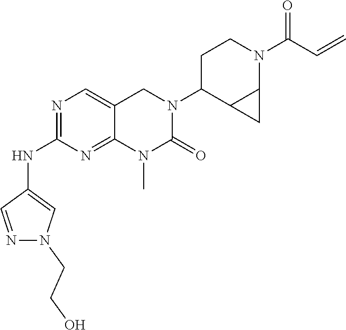 [SMILES: C=CC(=O)N1CCC(N2Cc3cnc(Nc4cnn(CCO)c4)nc3N(C)C2=O)C2CC21] | | |
| 116 | 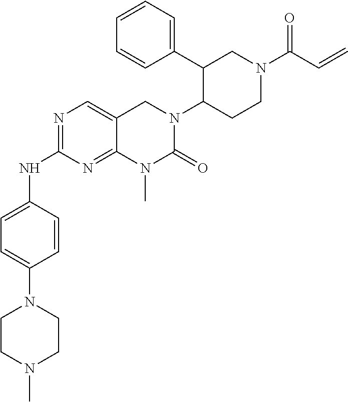 [SMILES: C=CC(=O)N1CCC(N2Cc3cnc(Nc4ccc(N5CCN(C)CC5)cc4)nc3N(C)C2=O)C(c2ccccc2)C1] | 567.2 | |

### Procedure for Preparation of Compound 126

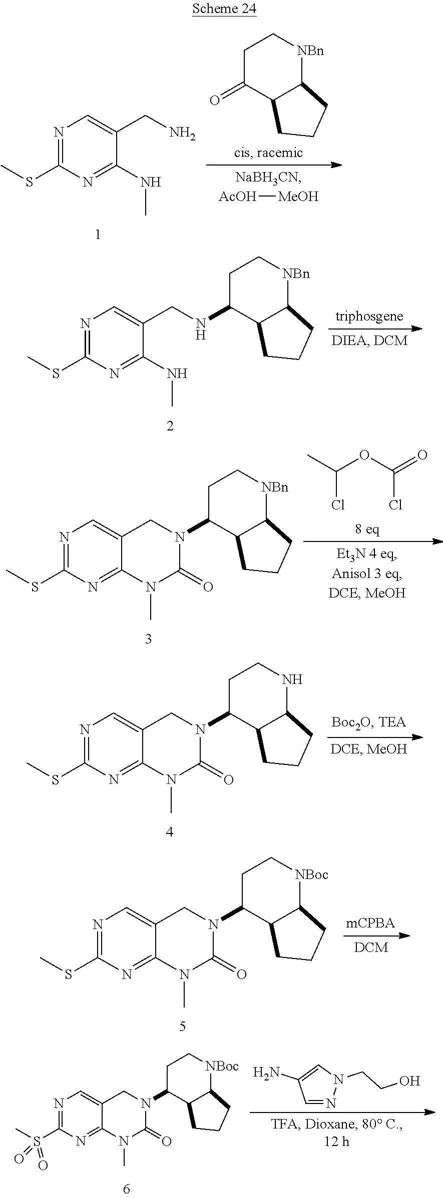

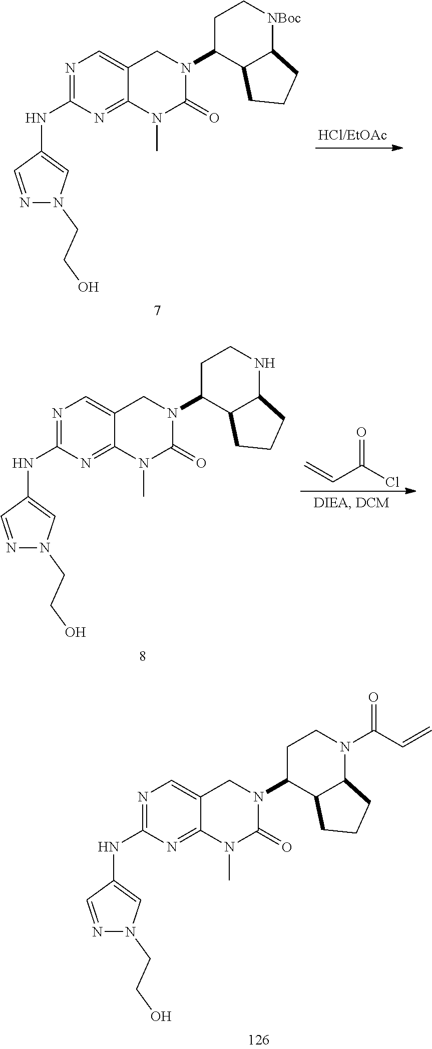

To a solution of 5-(aminomethyl)-N-methyl-2-methylsulfanyl-pyrimidin-4-amine (843.0 mg, 4.6 mmol, 1.5 eq) in MeOH (10 mL) was added 1-benzyl-3,4a, 5,6,7,7a-hexahydro-2H-cyclopenta[b]pyridin-4-one (700 mg, 3.1 mmol, 1 eq) and AcOH (1.1 g, 18.3 mmol, 1.1 mL, 6 eq). The mixture was stirred at 25° C. for 3 hr. Then NaBH₃CN (959.1 mg, 15.5 mmol, 5 eq) was added to the mixture, the mixture was stirred at 25° C. for 12 hr. LC-MS showed the desired compound was detected. The reaction mixture was concentrated under reduced pressure to remove methanol. The reaction mixture was partitioned between NaHCO₃ (50 mL) and ethyl acetate (50 mL×3). The organic phase was separated, washed with brine (30 mL×1), dried over Na₂SO₄, filtered and concentrated under reduced pressure to give a residue. The residue was purified by flash silica gel chromatography (20 g Silica Flash Column, Eluent of 0-20% Dichloromethane: Methanol @ 100 mL/min) to give 1-benzyl-N—[[4-(methylamino)-2-methylsulfanyl-pyrimidin-5-yl]methyl]-2,3,4,4a, 5,6,7,7a-octahydrocyclopenta[b]pyridin-4-amine (650 mg, 1.6 mmol, 53.6% yield) as a light yellow oil.

To a solution of 1-benzyl-N-[[4-(methylamino)-2-methylsulfanyl-pyrimidin-5-yl]methyl]-2,3,4,4a, 5,6,7,7a-octahydrocyclopenta[b]pyridin-4-amine (650 mg, 1.6 mmol, 1 eq) in DCM (20 mL) was added DIEA (1.1 g, 8.2 mmol, 1.4 mL, 5 eq) and triphosgene (485.2 mg, 1.6 mmol, 1 eq) at 0° C. The mixture was stirred at 25° C. for 12 hr. LC-MS showed the desired compound was detected. The residue was diluted with NaHCO₃ (50 mL) and extracted with dichloromethane (50 mL×3). The combined organic layers were washed with brine (30 mL×1), dried over Na₂SO₄, filtered and concentrated under reduced pressure to give a residue. The residue was purified by flash silica gel chromatography (20 g Silica Flash Column, Eluent of 0-15% Dichloromethane: Methanol @ 100 mL/min) to give 3-[(4aR)-1-benzyl-2,3,4,4a, 5,6,7,7a-octahydrocyclopenta[b]pyridin-4-yl]-1-methyl-7-methylsulfanyl-4H-pyrimido[4,5-d]pyrimidin-2-one (550 mg, 1.3 mmol, 79.4% yield) as a light yellow oil.

To a solution of 3-[(4aR)-1-benzyl-2,3,4,4a, 5,6,7,7a-octahydrocyclopenta[b]pyridin-4-yl]-1-methyl-7-methylsulfanyl-4H-pyrimido[4,5-d]pyrimidin-2-one (340 mg, 802.7 umol, 1 eq) in DCE (5 mL) was added TEA (324.9 mg, 3.2 mmol, 446.9 uL, 4 eq), anisole (260.4 mg, 2.4 mmol, 261.7 uL, 3 eq) and 1-chloroethyl carbonochloridate (918.1 mg, 6.4 mmol, 8 eq). The mixture was stirred at 80° C. for 24 hr. MeOH (5 mL) was added to the mixture, the mixture was stirred at 65° C. for 1 hr. LC-MS showed the desired compound was detected. The crude product 3-[(4aR)-2,3,4,4a, 5,6,7,7a-octahydro-1 H-cyclopenta[b]pyridin-4-yl]-1-methyl-7-methylsulfanyl-4H-pyrimido[4,5-d]pyrimidin-2-one (340 mg, crude) in DCE and MeOH as a black liquid was used into the next step without further work up.

To a solution of 3-[(4aR)-2,3,4,4a, 5,6,7,7a-octahydro-1 H-cyclopenta[b]pyridin-4-yl]-1-methyl-7-methylsulfanyl-4H-pyrimido[4,5-d]pyrimidin-2-one (340 mg, 1.0 mmol, 1 eq) in DCE (5 mL) and MeOH (5 mL) was added TEA (309.53 mg, 3.06 mmol, 425.76 uL, 3 eq) and Boc₂O (333.8 mg, 1.5 mmol, 351.4 uL, 1.5 eq). The mixture was stirred at 25° C. for 12 hr. LC-MS showed the desired compound was detected. The reaction mixture was concentrated under reduced pressure to give a residue. The reaction mixture was partitioned between H₂O (30 mL) and dichloromethane (30×3 mL). The organic phase was separated, washed with brine (20 mL×1), dried over Na₂SO₄, filtered and concentrated under reduced pressure to give a residue. The residue was purified by prep-TLC (SiO₂, Petroleum ether: Ethyl acetate=1:1) to give tert-butyl (4aS)-4-(1-methyl-7-methylsulfanyl-2-oxo-4H-pyrimido[4,5-d]pyrimidin-S-yl)-2,3,4,4a, 5,6,7,7a-octahydrocyclopenta[b]pyridine-1-carboxylate (160 mg, 369.0 umol, 36.2% yield) as a brown solid.

To a solution of tert-butyl (4aS)-4-(1-methyl-7-methylsulfanyl-2-oxo-4H-pyrimido[4,5-d]pyrimidin-S-yl)-2,3,4,4a, 5,6,7,7a-octahydrocyclopenta[b]pyridine-1-carboxylate (141 mg, 325.2 umol, 1 eq) in DCM (2 mL) was added mCPBA (99.0 mg, 487.8 umol, 85% purity, 1.5 eq). The mixture was stirred at 0° C. for 2 hr. LC-MS showed the desired compound was detected. The reaction mixture was partitioned between Na₂SO₃ (20 mL) and dichloromethane (20×3 mL). The organic phase was separated, washed with NaHCO₃ (20 mL×3), dried over Na₂SO₄, filtered and concentrated under reduced pressure to give tert-butyl (4aS)-4-(1-methyl-7-methylsulfonyl-2-oxo-4H-pyrimido[4,5-d]pyrimidin-S-yl)-2,3,4,4a, 5,6,7,7a-octahydrocyclopenta[b]pyridine-1-carboxylate (150 mg, crude) as a white solid.

To a solution of tert-butyl (4aS)-4-(1-methyl-7-methylsulfonyl-2-oxo-4H-pyrimido[4,5-d]pyrimidin-S-yl)-2,3,4,4a, 5,6,7,7a-octahydrocyclopenta[b]pyridine-1-carboxylate (50 mg, 107.4 umol, 1 eq) in dioxane (2 mL) was added TFA (18.4 mg, 161.1 umol, 11.9 uL, 1.5 eq) and 2-(4-aminopyrazol-1-yl)ethanol (16.4 mg, 128.8 umol, 1.2 eq). The mixture was stirred at 80° C. for 12 hr. LC-MS showed the desired compound was detected. The reaction mixture was concentrated under reduced pressure to remove solvent. The residue was purified by prep-HPLC (FA condition; column: Phenomenex Luna C18 200×40 mm×10 um;mobile phase: [water(FA)-ACN];B %: 25%-55%, 8 min) to give tert-butyl 4-[7-[[1-(2-hydroxyethyl)pyrazol-4-yl]amino]-1-methyl-2-oxo-4H-pyrimido[4,5-d]pyrimidin-S-yl]-2,3,4,4a, 5,6,7,7a-octahydrocyclopenta[b]pyridine-1-carboxylate (60 mg, 117.1 umol, 54.5% yield) as a yellow solid.

A solution of tert-butyl 4-[7-[[1-(2-hydroxyethyl)pyrazol-4-yl]amino]-1-methyl-2-oxo-4H-pyrimido[4,5-d]pyrimidin-S-yl]-2,3,4,4a, 5,6,7,7a-octahydrocyclopenta[b]pyridine-1-carboxylate (50 mg, 97.5 umol, 1 eq) in HCl/EtOAc (4 mL, 4 M) was stirred at 25° C. for 10 min. LC-MS showed the desired compound was detected. The reaction mixture was concentrated under reduced pressure to give crude product 3-(2,3,4,4a, 5,6,7,7a-octahydro-1H-cyclopenta[b]pyridin-4-yl)-7-[[1-(2-hydroxyethyl)pyrazol-4-yl]amino]-1-methyl-4H-pyrimido[4,5-d]pyrimidin-2-one (60 mg, crude) obtained as yellow solid used into the next step without further purification.

To a solution of 3-(2,3,4,4a, 5,6,7,7a-octahydro-1 H-cyclopenta[b]pyridin-4-yl)-7-[[1-(2-hydroxyethyl)pyrazol-4-yl]amino]-1-methyl-4H-pyrimido[4,5-d]pyrimidin-2-one (30 mg, 66.8 umol, 1 eq, HCl) and DIEA (25.9 mg, 200.5 umol, 34.9 uL, 3 eq) in DCM (2 mL) was added a solution of prop-2-enoyl chloride (6.05 mg, 66.8 umol, 5.45 uL, 1 eq) in DCM (0.2 mL) and at 0° C. The reaction was stirred at 25° C. for 1 hr. LC-MS showed the desired compound was detected. The reaction mixture was concentrated under reduced pressure to remove the solvent. The residue was purified by prep-HPLC (FA condition; column: Phenomenex Luna C18 200×40 mm×10 um;mobile phase: [water(FA)-ACN];B %: 10%-40%, 8 min) to give 7-[[1-(2-hydroxyethyl)pyrazol-4-yl]amino]-1-methyl-S-(1-prop-2-enoyl-2,3,4,4a, 5,6,7,7a-octahydrocyclopenta[b]pyridin-4-yl)-4H-pyrimido[4,5-d]pyrimidin-2-one (10 mg, 21.4 umol, 32.1% yield, 100% purity) as a white solid.

### Procedure for Preparation of Compound 123

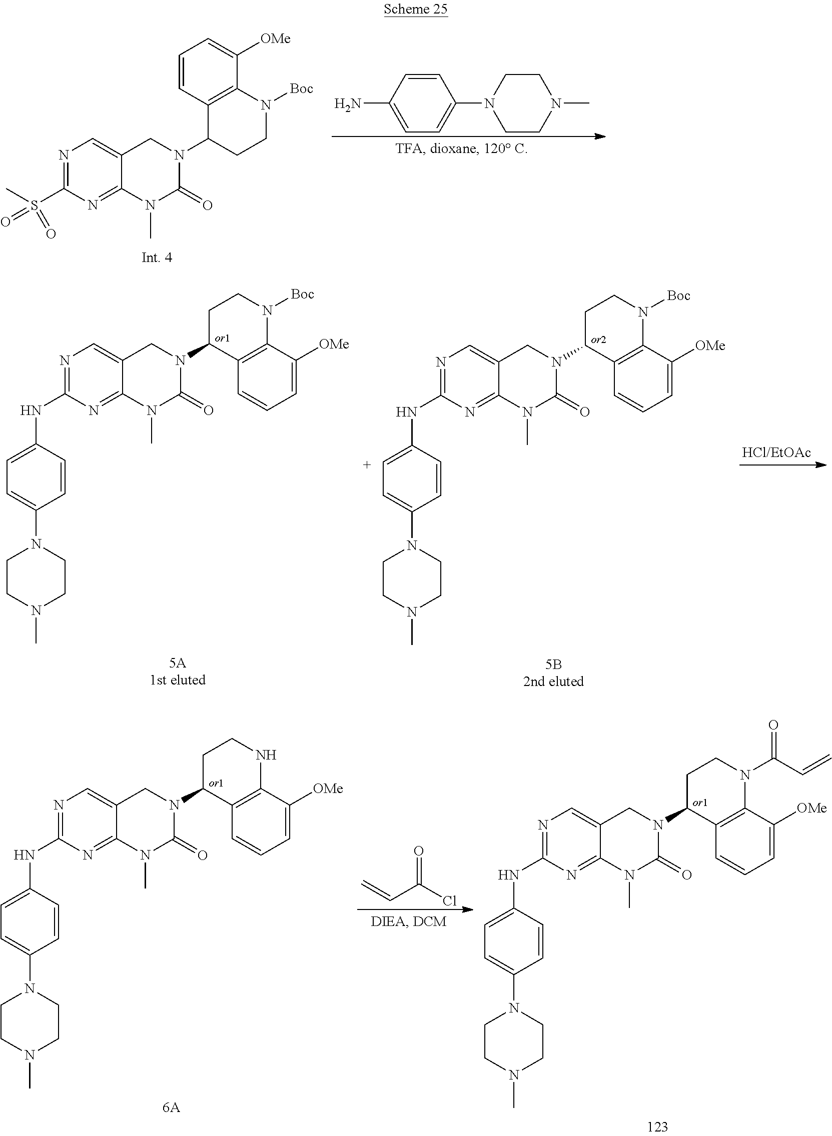

4-(4-methylpiperazin-1-yl)aniline (91.2 mg, 476.6 umol, 1.2 eq) in dioxane (2 mL) was added tert-butyl 8-methoxy-4-(1-methyl-7-methylsulfonyl-2-oxo-4H-pyrimido[4,5-d]pyrimidin-3-yl)-3,4-dihydro-2H-quinoline-1-carboxylate (200 mg, 397.2 umol, 1 eq) and TFA (68.0 mg, 595.8 umol, 44.1 uL, 1.5 eq) (8×batch). The reaction mixture was stirred at 120° C. for 12 hr. LC-MS showed desired mass was detected. The reaction mixture was concentrated under reduced pressure to give a residue. The reaction mixture was diluted with dimethyl sulfoxide (10 mL) and filtered. The residue was purified by prep-HPLC (FA condition; column: Phenomenex Luna C18 200×40 mm×10 um;mobile phase:[water(FA)-ACN];B %: 5%-45%, 8 min) to isolate the racemic product, which was separated by SFC (column: DAICEL CHIRALCEL OX(250 mm×30 mm, 10 um); mobile phase:[0.1% NH₃H₂O MeOH]; B %: 50%-50%, 9 min)) to give tert-butyl rel-(4S)-8-methoxy-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate(400 mg, 650.7 umol, 18.2% yield) (Rt=2.670 min) and tert-butyl rel-(4R)-8-methoxy-4-[1-methyl-7-[4-(4-methylpiperazin-1-yl)anilino]-2-oxo-4H-pyrimido[4,5-d]pyrimidin-3-yl]-3,4-dihydro-2H-quinoline-1-carboxylate (400 mg, 650.7 umol, 18.2% yield) (Rt=3.210 min).

A solution of tert-butyl rel-(4S)-8-methoxy-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (150 mg, 244.0 umol, 1 eq) in HCl/EtOAc (4 mL, 4 M) was stirred at 25° C. for 10 min. The reaction was monitored by LCMS which showed the reactant was consumed completely and the desired mass peak was detected. The reaction mixture was concentrated under reduced pressure to give 3-[rel-(4S)-8-methoxy-1,2,3,4-tetrahydroquinolin-4-yl]-1-methyl-7-[4-(4-methylpiperazin-1-yl)anilino]-4H-pyrimido[4,5-d]pyrimidin-2-one (350 mg, crude) as yellow solid. The crude product was used into the next step without further purification.

To a solution of 3-[rel-(4S)-8-methoxy-1,2,3,4-tetrahydroquinolin-4-yl]-1-methyl-7-[4-(4-methylpiperazin-1-yl)anilino]-4H-pyrimido[4,5-d]pyrimidin-2-one (70 mg, 127.0 umol, 1 eq, HCl) in DCM (2 mL) was added DIEA (49.3 mg, 381.1 umol, 66.4 uL, 3 eq) and prop-2-enoyl chloride (13.8 mg, 152.4 umol, 12.4 uL, 1.2 eq) at 0° C. The mixture was stirred at 25° C. for 1 hr. The reaction was monitored by LCMS which showed complete consumption of reactant and detection of the desired mass peak. The reaction mixture was concentrated under reduced pressure to remove solvent. The residue was diluted with DMF (3 mL). The residue was purified by prep-HPLC (FA condition; column: Phenomenex Luna 80×30 mm×3 um; mobile phase:[water(FA)-ACN];B %: 15%-40%, 8 min) to yield 3-[rel-(4S)-8-methoxy-1-prop-2-enoyl-3,4-dihydro-2H-quinolin-4-yl]-1-methyl-7-[4-(4-methylpiperazin-1-yl)anilino]-4H-pyrimido[4,5-d]pyrimidin-2-one (10 mg, 15.0 umol, 11.8% yield, 91.7% purity, FA) as a white solid.

### Procedure for Preparation of Compound 124

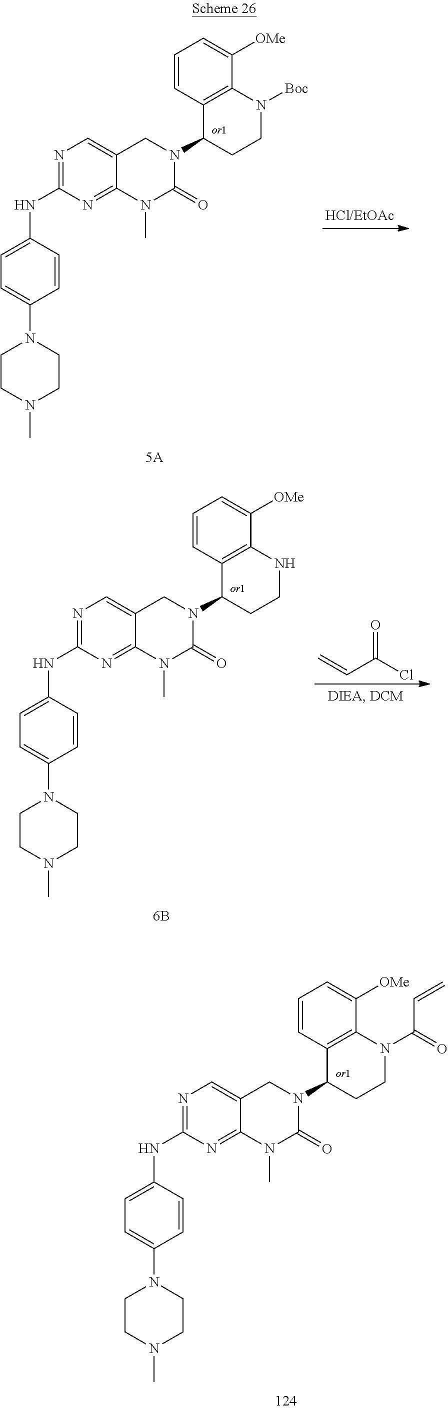

A mixture of tert-butyl rel-(4R)-8-methoxy-4-[1-methyl-7-[4-(4-methylpiperazin-1-yl)anilino]-2-oxo-4H-pyrimido [4,5-d]pyrimidin-S-yl]-3,4-dihydro-2H-quinoline-1-carboxylate (306 mg, 497.8 umol, 1 eq) in HCl/EtOAc (3 mL, 4 M) was stirred at 20° C. for 1 hr. LC-MS showed the desired compound was detected. The reaction mixture was filtrated and then concentrated under reduced pressure to remove solvent to give 3-[rel-(4R)-8-methoxy-1,2,3,4-tetrahydroquinolin-4-yl]-1-methyl-7-[4-(4-methylpiperazin-1-yl)anilino]-4H-pyrimido[4, 5-d]pyrimidin-2-one (260 mg, crude, HCl) as a brown solid.

To a solution of 3-[rel-(4R)-8-methoxy-1,2,3,4-tetrahydroquinolin-4-yl]-1-methyl-7-[4-(4-methylpiperazin-1-yl)anilino]-4H-pyrimido[4,5-d]pyrimidin-2-one (50 mg, 90.7 umol, 1 eq, HCl) in DCM (2 mL) was added DIEA (35.2 mg, 272.2 umol, 47.4 uL, 3 eq) and prop-2-enoyl chloride (9.8 mg, 108.9 umol, 8.9 uL, 1.2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition) column: Phenomenex Luna C18 200×40 mm×10 um; mobile phase: [water(FA)-ACN]; B %: 5%-40%, 8 min to give 3-[rel-(4R)-8-methoxy-1-prop-2-enoyl-3,4-dihydro-2H-quinolin-4-yl]-1-methyl-7-[4-(4-methylpiperazin-1-yl)anilino]-4H-pyrimido[4,5-d]pyrimidin-2-one (5.3 mg, 8.6 umol, 9.5% yield, 100% purity, FA) as a white solid.

### Procedure for Preparation of Compound 122

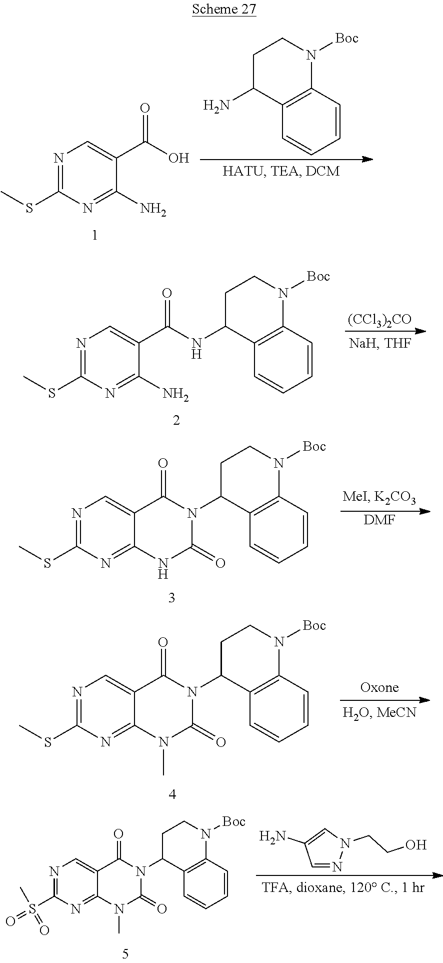

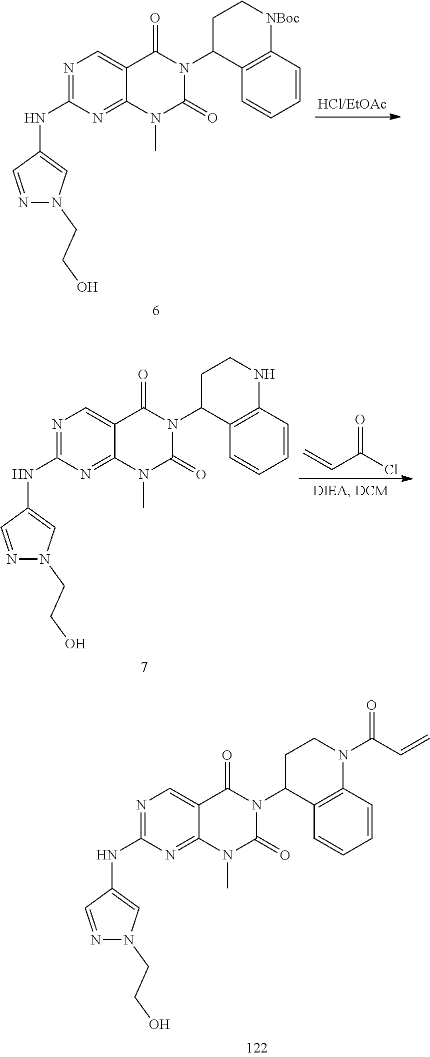

To a solution of 4-amino-2-methylsulfanyl-pyrimidine-5-carboxylic acid (2.2 g, 11.8 mmol, 1 eq) in DCM (50 mL) was added tert-butyl 4-amino-3, 4-dihydro-2H-quinoline-1-carboxylate (2.9 g, 11.8 mmol, 1 eq), HATU (6.7 g, 17.7 mmol, 1.5 eq) and TEA (3.6 g, 35.3 mmol, 4.9 mL, 3 eq). The mixture was stirred at 20° C. for 12 hr. LC-MS showed desired compound was detected. The residue was diluted with H₂O 200 mL and extracted with dichloromethane (200 mL×3). The combined organic layers were washed with brine (150 mL×1), dried over Na₂SO₄, filtered and concentrated under reduced pressure to give a residue. The residue was purified by flash silica gel chromatography (40 g Silica Flash Column, Eluent of 0-30% Ethyl acetate/Petroleum ether gradient @ 120 mL/min) to give tert-butyl 4-[(4-amino-2-methylsulfanyl-pyrimidine-5-carbonyl)amino]-3,4-dihydro-2H-quinoline-1-carboxylate (2 g, 4.8 mmol, 40.9% yield) as a yellow solid.

To a solution of tert-butyl 4-[(4-amino-2-methylsulfanyl-pyrimidine-5-carbonyl)amino]-3,4-dihydro-2H-quinoline-1-carboxylate (2 g, 4.8 mmol, 1 eq) in THE (40 mL) was added NaH (1.2 g, 28.9 mmol, 60% purity, 6 eq) at 0° C. Then bis (trichloromethyl) carbonate (1.4 g, 4.8 mmol, 1 eq) was added to the mixture at 0° C. The mixture was stirred at 20° C. for 12 hr. LC-MS showed desired compound was detected. The residue was diluted with NaHCO₃ 100 mL and extracted with ethyl acetate (50 mL×3). The combined organic layers were washed with brine (30 mL×1), dried over Na₂SO₄, filtered and concentrated under reduced pressure to give a residue. The residue was purified by flash silica gel chromatography (40 g Silica Flash Column, Eluent of 0-15% Ethyl acetate/Petroleum ether gradient @ 120 mL/min) to give tert-butyl 4-(2-methylsulfanyl-5,7-dioxo-8H-pyrimido[4,5-d]pyrimidin-6-yl)-3,4-dihydro-2H-quinoline-1-carboxylate (925 mg, 2.1 mmol, 43.5% yield) as a yellow oil.

To a solution of tert-butyl 4-(2-methylsulfanyl-5,7-dioxo-8H-pyrimido[4,5-d]pyrimidin-6-yl)-3,4-dihydro-2H-quinoline-1-carboxylate (925 mg, 2.10 mmol, 1 eq) in DMF (10 mL) was added K₂CO₃ (868.7 mg, 6.3 mmol, 3 eq) and Mel (356.8 mg, 2.5 mmol, 156.5 uL, 1.2 eq). The mixture was stirred at 20° C. for 12 hr. LC-MS showed desired compound was detected. The reaction mixture was quenched by addition H₂O 100 mL, and extracted with ethyl acetate (100 mL×7). The combined organic layers were washed with brine 80 mL, dried over Na₂SO₄, filtered and concentrated under reduced pressure to give a residue. The residue was purified by flash silica gel chromatography (20 g Silica Flash Column, Eluent of 0-9% Ethyl acetate/Petroleum ether gradient @ 120 mL/min) to give tert-butyl 4-(1-methyl-7-methylsulfanyl-2,4-dioxo-pyrimido[4,5-d]pyrimidin-S-yl)-3,4-dihydro-2H-quinoline-1-carboxylate (400 mg, 878.1 umol, 41.9% yield) as a yellow solid.

To a solution of tert-butyl 4-(1-methyl-7-methylsulfanyl-2,4-dioxo-pyrimido[4,5-d]pyrimidin-S-yl)-3,4-dihydro-2H-quinoline-1-carboxylate (400 mg, 878.1 umol, 1 eq) in MeCN (2 mL)/H₂O (2 mL) was added Oxone (1.1 g, 1.8 mmol, 2 eq). The mixture was stirred at 20° C. for 12 hr. LC-MS showed desired compound was detected. The reaction mixture was concentrated under reduced pressure to remove solvent. The reaction mixture was partitioned between Na₂SO₃ 200 mL and DCM (200×3 mL). The organic phase was separated, washed with NaHCO₃ (150 mL×3), dried over Na₂SO₄, filtered and concentrated under reduced pressure to give tert-butyl 4-(1-methyl-7-methylsulfonyl-2,4-dioxo-pyrimido[4,5-d]pyrimidin-S-yl)-3,4-dihydro-2H-quinoline-1-carboxylate (328 mg, crude) as a brown solid.

To a solution of tert-butyl 4-(1-methyl-7-methylsulfonyl-2,4-dioxo-pyrimido[4,5-d]pyrimidin-S-yl)-3,4-dihydro-2H-quinoline-1-carboxylate (164 mg, 336.4 umol, 1 eq) in dioxane (1.5 mL) was added TFA (57.5 mg, 504.6 umol, 37.4 uL, 1.5 eq) and 2-(4-aminopyrazol-1-yl)ethanol (51.3 mg, 403.7 umol, 1.2 eq). The mixture was stirred at 120° C. for 1 hr. LC-MS showed desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 30%-65%, 8 min to give tert-butyl 4-[7-[[1-(2-hydroxyethyl)pyrazol-4-yl]amino]-1-methyl-2,4-dioxo-pyrimido[4,5-d]pyrimidin-S-yl]-3,4-dihydro-2H-quinoline-1-carboxylate (60 mg, 112.2 umol, 16.7% yield) as a yellow solid.

A mixture of tert-butyl 4-[7-[[1-(2-hydroxyethyl)pyrazol-4-yl]amino]-1-methyl-2,4-dioxo-pyrimido[4,5-d]pyrimidin-S-yl]-3,4-dihydro-2H-quinoline-1-carboxylate (60 mg, 112.2 umol, 1 eq) in HCl/EtOAc (1 mL, 4 M) was stirred at 20° C. for 1 hr. LC-MS showed desired compound was detected. The reaction mixture was filtered and concentrated under reduced pressure to give 7-[[1-(2-hydroxyethyl)pyrazol-4-yl]amino]-1-methyl-S-(1,2,3,4-tetrahydroquinolin-4-yl)pyrimido[4,5-d]pyrimidine-2,4-dione (50 mg, crude, HCl) as a yellow solid.

To a solution of 7-[[1-(2-hydroxyethyl)pyrazol-4-yl]amino]-1-methyl-S-(1,2,3,4-tetrahydroquinolin-4-yl)pyrimido[4,5-d]pyrimidine-2,4-dione (50 mg, 106.2 umol, 1 eq, HCl) and DIEA (41.2 mg, 318.5 umol, 55.5 uL, 3 eq) in DCM (1 mL) was added prop-2-enoyl chloride (9.6 mg, 106.2 umol, 8.7 uL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 20%-55%, 8 min to give 7-[[1-(2-hydroxyethyl)pyrazol-4-yl]amino]-1-methyl-S-(1-prop-2-enoyl-3,4-dihydro-2H-quinolin-4-yl)pyrimido[4,5-d]pyrimidine-2,4-dione (15.3 mg, 31.3 umol, 29.5% yield, 100% purity) as a yellow solid.

### Procedure for Preparation of Compound 117

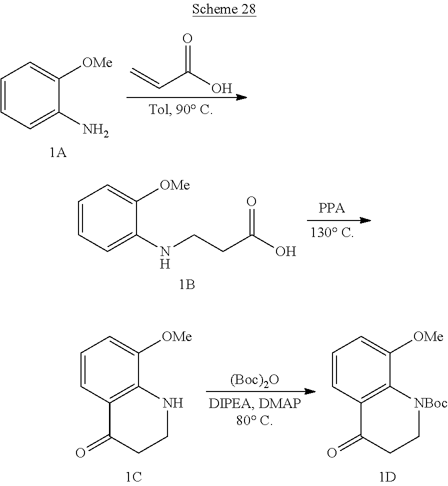

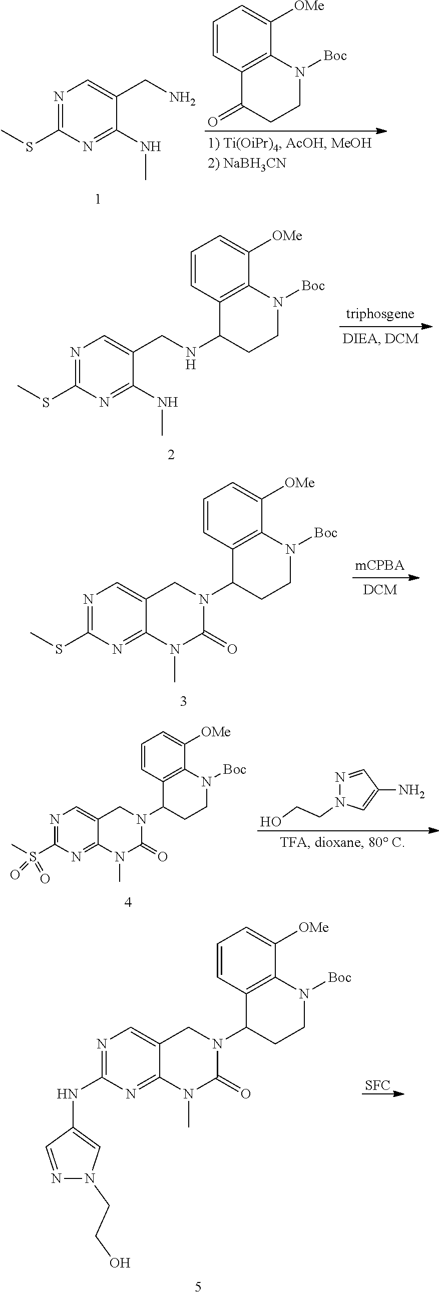

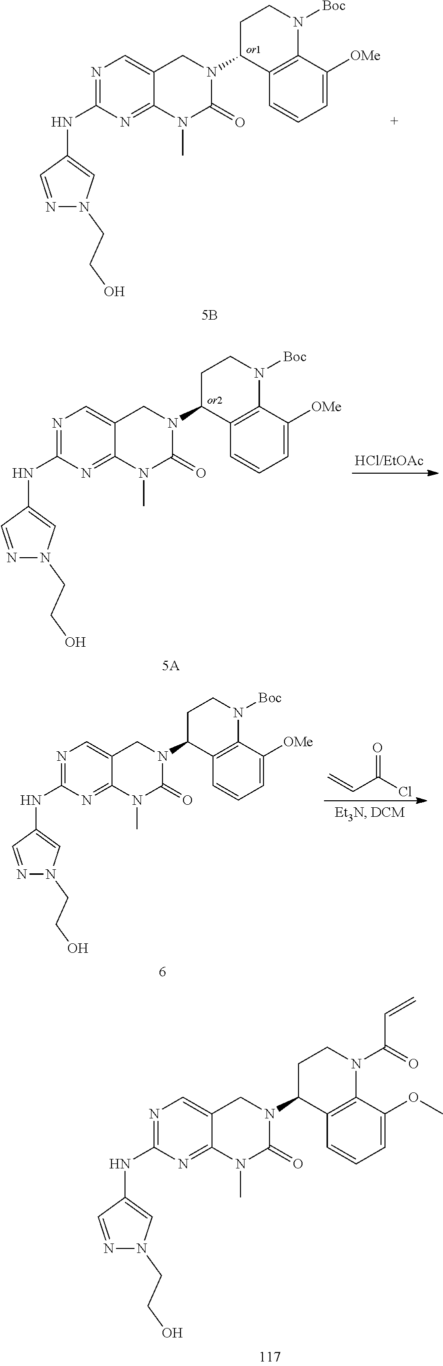

To a solution of 2-methoxyaniline (10 g, 81.2 mmol, 9.2 mL, 1 eq) in Tol. (200 mL) was added acrylic acid (5.9 g, 81.2 mmol, 5.6 mL, 1 eq). The mixture was stirred at 90° C. for 12 hr. LC-MS showed desired mass was detected. The reaction mixture was concentrated under reduced pressure to give a residue. The residue was purified by flash silica gel chromatography (40 g Silica Flash Column, Eluent of 0-50% Ethyl acetate/Petroleum ether gradient @ 120 mL/min) to yield 3-(2-methoxyanilino)propanoic acid (15 g, 76.8 mmol, 47.3% yield) as a brown solid.

A mixture of 3-(2-methoxyanilino)propanoic acid (12 g, 61.5 mmol, 1 eq) in PPA (200 mL) was stirred at 130° C. for 3 hr. The reaction was monitored by LCMS which showed the reactant was consumed completely and the desired mass peak was detected. The reaction mixture was diluted with H₂O (2000 mL) and set it to neutral with sodium hydroxide. The residue was extracted with dichloromethane (2000 mL×3). The combined organic layers were dried over Na₂SO₄, filtered and concentrated under reduced pressure to give a residue. The residue was purified by flash silica gel chromatography (10 g Silica Flash Column, Eluent of 0-100% Ethyl acetate/Petroleum ether gradient @ 150 mL/min) to give 8-methoxy-2,3-dihydro-1H-quinolin-4-one(8.7 g, 49.1 mmol, 40.0% yield) as a black solid.

To a solution of 8-methoxy-2,3-dihydro-1 H-quinolin-4-one (1 g, 5.6 mmol, 1 eq) was added Boc₂O (8 g, 36.7 mmol, 8.4 mL, 6.5 eq), DMAP (69.0 mg, 564.3 umol, 0.1 eq) and DIEA (1.1 g, 8.5 mmol, 1.5 mL, 1.5 eq). The reaction mixture was stirred at 80° C. for 2 hr. The reaction was monitored by LCMS which showed complete consumption of reactant and detection of the desired mass peak. The residue was purified by flash silica gel chromatography (20 g Silica Flash Column, Eluent of 0-15% Ethyl acetate/Petroleum ether gradient @ 80 mL/min) to give the crude product tert-butyl 8-methoxy-4-oxo-2,3-dihydroquinoline-1-carboxylate (7 g, 25.2 mmol, 56.0% yield) as a yellow solid.

To a solution of tert-butyl 8-methoxy-4-oxo-2,3-dihydroquinoline-1-carboxylate (4.4 g, 15.9 mmol, 1 eq) in MeOH (30 mL) was added 5-(aminomethyl)-N-methyl-2-methylsulfanyl-pyrimidin-4-amine (3.0 g, 15.9 mmol, 1 eq), Ti(i-PrO)₄ (6.8 g, 23.8 mmol, 7.0 mL, 1.5 eq) and then AcOH (5.7 g, 95.2 mmol, 5.4 mL, 6 eq) was added to the mixture to adjust pH=5 and the mixture was stirred at 25° C. for 2 hr. NaBH₃CN (3.0 g, 47.6 mmol, 3 eq) was added to the mixture. The mixture was stirred at 65° C. for 12 hr. The reaction was monitored by LCMS which showed complete consumption of reactant and detection of the desired mass peak. The reaction mixture was concentrated under reduced pressure to remove solvent. The residue was diluted with NaHCO₃ (30 mL) and extracted with dichloromethane (30 mL×3). The combined organic layers were washed with brine (30 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 100 mL/min) to yield Compound tert-butyl 8-methoxy-4-[[4-(methylamino)-2-methylsulfanyl-pyrimidin-5-yl]methylamino]-3,4-dihydro-2H-quinoline-1-carboxylate(7 g, 15.7 mmol, 99.0% yield) as a yellow solid.

To a solution of tert-butyl 8-methoxy-4-[[4-(methylamino)-2-methylsulfanyl-pyrimidin-5-yl]methylamino]-3,4-dihydro-2H-quinoline-1-carboxylate (2.9 g, 6.5 mmol, 1 eq) in DCM (15 mL) was added DIEA (2.5 g, 19.5 mmol, 3.4 mL, 3 eq) and bis(trichloromethyl) carbonate (1.6 g, 5.2 mmol, 0.8 eq) at 0° C. The mixture was stirred at 25° C. for 12 hr. The reaction was monitored by LCMS which showed complete consumption of reactant and detection of the desired product. The residue was diluted with NaHCO₃ (30 mL) and extracted with Dichloromethane (50 mL×3). The combined organic layers were washed with brine(50 mL×1), dried over Na₂SO₄, filtered and concentrated under reduced pressure to give compound tert-butyl 8-methoxy-4-(1-methyl-7-methylsulfanyl-2-oxo-4H-pyrimido[4,5-d]pyrimidin-S-yl)-3,4-dihydro-2H-quinoline-1-carboxylate(3.8 g, crude) as yellow oil.

To a solution of tert-butyl 8-methoxy-4-(1-methyl-7-methylsulfanyl-2-oxo-4H-pyrimido[4,5-d]pyrimidin-S-yl)-3,4-dihydro-2H-quinoline-1-carboxylate (3 g, 6.4 mmol, 1 eq) in DCM (8 mL) was added m-CPBA (2.3 g, 11.5 mmol, 85% purity, 1.8 eq) at 0° C. The mixture was stirred at 25° C. for 1.5 hr. The reaction was monitored by LCMS which showed complete consumption of reactant and detection of the desired product. The residue was diluted with Na₂SO₃ (10%) (10 mL) and extracted with Dichloromethane (20 mL×3). The combined organic layers were washed with NaHCO₃ (20 mL×3), dried over Na₂SO₄, filtered and concentrated under reduced pressure to give tert-butyl 8-methoxy-4-(1-methyl-7-methylsulfonyl-2-oxo-4H-pyrimido[4,5-d]pyrimidin-S-yl)-3,4-dihydro-2H-quinoline-1-carboxylate(3.8 g, crude) as a white solid.

To a solution of 2-(4-aminopyrazol-1-yl)ethanol (65.6 mg, 516.3 umol, 1.3 eq) in dioxane (2 mL) was added tert-butyl 8-methoxy-4-(1-methyl-7-methylsulfonyl-2-oxo-4H-pyrimido[4,5-d]pyrimidin-S-yl)-3,4-dihydro-2H-quinoline-1-carboxylate (200 mg, 397.2 umol, 1 eq) and TFA (68.0 mg, 595.8 umol, 44.1 uL, 1.5 eq). The reaction mixture was stirred at 80° C. for 12 hr (8×batch). The reaction was monitored by LCMS which showed complete consumption of reactant and detection of the desired product. The residue was purified by prep-HPLC (FA condition; column: Phenomenex Luna C18 200×40 mm×10 um;mobile phase: [water(FA)-ACN];B %: 15%-45%, 8 min). to isolate the racemic product. The product was separated by SFC (column: DAICEL CHIRALCEL OX (250 mm×30 mm, 10 um);mobile phase: [0.1% NH₃H₂O MeOH];B %: 50%-50%, 9 min)) to give tert-butyl rel-(4S)-4-[7-[[1-(2-hydroxyethyl)pyrazol-4-yl]amino]-1-methyl-2-oxo-4H-pyrimido[4,5-d]pyrimidin-S-yl]-8-methoxy-3,4-dihydro-2H-quinoline-1-carboxylate (550 mg, 998.9 umol, 50.3% yield) (Rt=1.914 min) and tert-butyl rel-(4R)-4-[7-[[1-(2-hydroxyethyl)pyrazol-4-yl]amino]-1-methyl-2-oxo-4H-pyrimido[4,5-d]pyrimidin-S-yl]-8-methoxy-3,4-dihydro-2H-quinoline-1-carboxylate (450 mg, 817.3 umol, 41.2% yield) (Rt=2.108 min).

A solution of tert-butyl rel-(4S)-4-[7-[[1-(2-hydroxyethyl)pyrazol-4-yl]amino]-1-methyl-2-oxo-4H-pyrimido[4,5-d]pyrimidin-S-yl]-8-methoxy-3,4-dihydro-2H-quinoline-1-carboxylate (520 mg, 944.4 umol, 1 eq) in HCl/EtOAc (4 mL, 4 M) was stirred at 25° C. for 10 min. The reaction was monitored by LCMS which showed complete consumption of reactant and detection of the desired product. The reaction mixture was concentrated under reduced pressure to give 7-[[1-(2-hydroxyethyl)pyrazol-4-yl]amino]-S-[rel-(4S)-8-methoxy-1,2,3,4-tetrahydroquinolin-4-yl]-1-methyl-4H-pyrimido[4,5-d]pyrimidin-2-one(600 mg, crude) as yellow solid. The crude product was used into the next step without further purification.

To a solution of 7-[[1-(2-hydroxyethyl)pyrazol-4-yl]amino]-S-[rel-(4S)-8-methoxy-1,2,3,4-tetrahydroquinolin-4-yl]-1-methyl-4H-pyrimido[4,5-d]pyrimidin-2-one (50 mg, 111.0 umol, 1 eq) in DCM(2 mL) was added DIEA (43.0 mg, 333.0 umol, 58.0 uL, 3 eq) and prop-2-enoyl chloride (12.1 mg, 133.2 umol, 10.9 uL, 1.2 eq) at 0° C. The mixture was stirred at 25° C. for 1 hr. The reaction was monitored by LCMS which showed complete consumption of reactant and detection of the desired mass peak. The reaction mixture was concentrated under reduced pressure to remove solvent. The residue was diluted with dimethyl formamide (3 mL). The residue was purified by prep-HPLC (FA condition; column: Phenomenex Luna C18 200×40 mm×10 um;mobile phase: [water(FA)-ACN];B %: (1%-35%, 8 min)) to yield 1-cyclopentyl-7-[4-(4-methylpiperazin-1-yl)anilino]-S-(1-prop-2-enoyl-3,4-dihydro-2H-quinolin-4-yl)-4H-pyrimido[4,5-d]pyrimidin-2-one(5 mg, 8.3 umol, 5.2% yield, 97.9% purity) as a white solid.

### General Procedure for Preparation of Compound 119

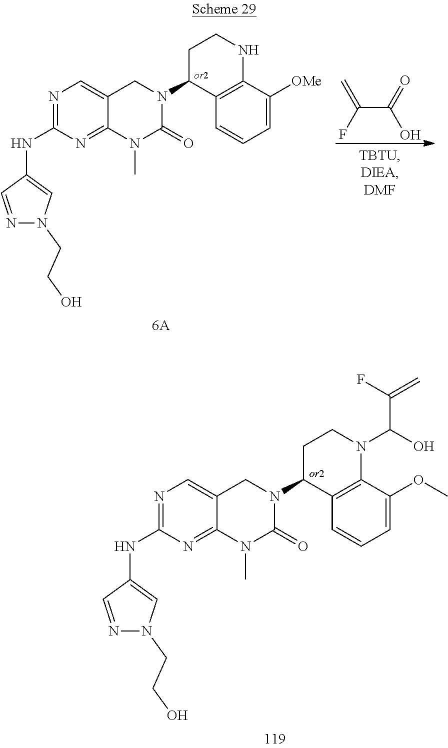

To a solution of 2-fluoroprop-2-enoic acid(24.0 mg, 266.4 umol, 1.2 eq) in DMF(2 mL) was added 7-[[1-(2-hydroxyethyl)pyrazol-4-yl]amino]-S-[rel-(4S)-8-methoxy-1,2,3,4-tetrahydroquinolin-4-yl]-1-methyl-4H-pyrimido[4,5-d]pyrimidin-2-one(100 mg, 222.0 umol, 1 eq), DIEA(86.1 mg, 666.0 umol, 116.0 uL, 3 eq) and TBTU(92.7 mg, 288.6 umol, 1.3 eq). The mixture was stirred at 25° C. for 12 hr. LC-MS showed desired mass was detected. The reaction mixture was concentrated under reduced pressure to remove solvent. The residue was diluted with methanol (2 mL) and dimethyl formamide (1 mL). The residue was purified by prep-HPLC(FA condition; column: column: Phenomenex Luna C18 200×40 mm×10 um;mobile phase: [water(FA)-ACN]; B %: 15%-50%, 8 min) to yield 3-[rel-(4S)-1-(2-fluoroprop-2-enoyl)-8-methoxy-3,4-dihydro-2H-quinolin-4-yl]-7-[[1-(2-hydroxyethyl)pyrazol-4-yl]amino]-1-methyl-4H-pyrimido[4,5-d]pyrimidin-2-one(25 mg, 47.5 umol, 21.4% yield, 99.4% purity) obtained as a white solid.

### General Procedure for Preparation of Compound 120

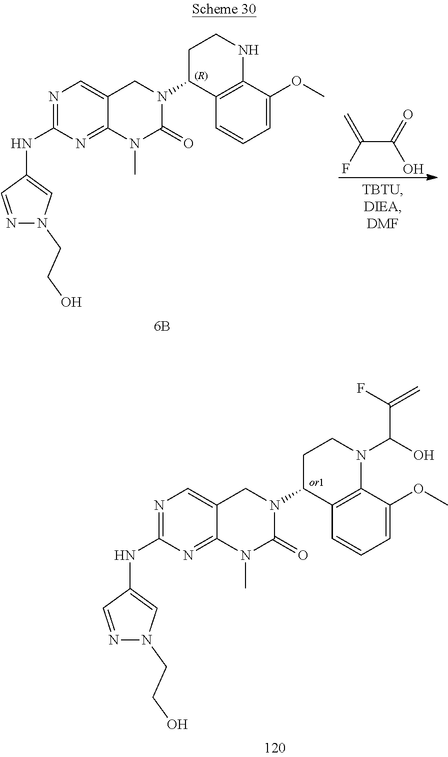

To a solution of 7-[[1-(2-hydroxyethyl)pyrazol-4-yl]amino]-S-[rel-(4R)-8-methoxy-1,2,3,4-tetrahydroquinolin-4-yl]-1-methyl-4H-pyrimido[4,5-d]pyrimidin-2-one (100 mg, 222.0 umol, 1 eq) in (1 mL) was added TBTU (71.3 mg, 222.0 umol, 1 eq), DIEA (86.1 mg, 665.9 umol, 116.0 uL, 3 eq) and 2-fluoroprop-2-enoic acid (24.0 mg, 266.4 umol, 1.2 eq). The mixture was stirred at 20° C. for 12 hr. LC-MS showed the desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 5%-45%, 8 min to give 3-[rel-(4R)-1-(2-fluoroprop-2-enoyl)-8-methoxy-3,4-dihydro-2H-quinolin-4-yl]-7-[[1-(2-hydroxyethyl)pyrazol-4-yl]amino]-1-methyl-4H-pyrimido[4,5-d]pyrimidin-2-one (25.2 mg, 48.2 umol, 21.7% yield, 100% purity) as a brown solid.

### Procedure for Preparation of Compound 125

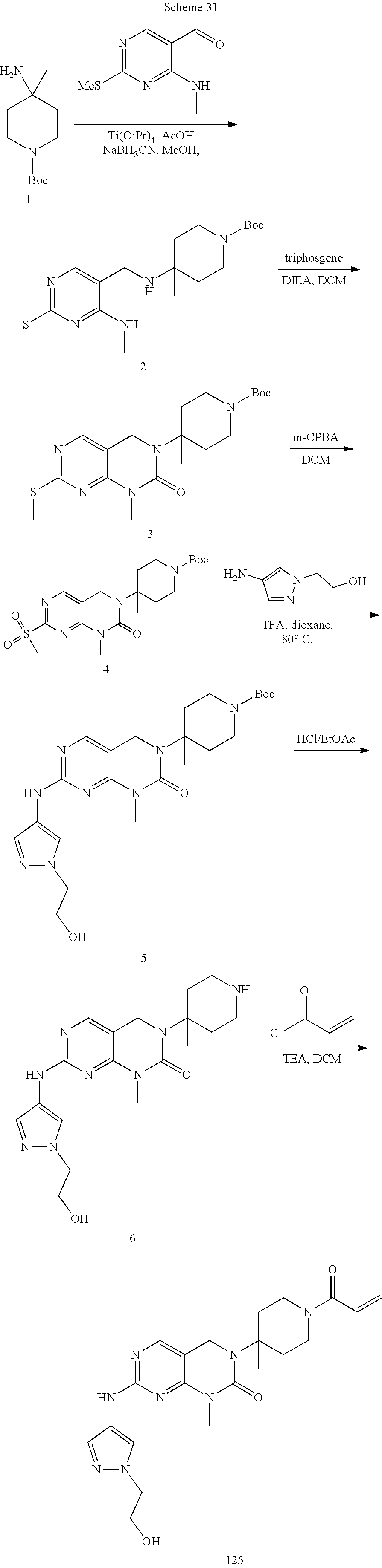

To a solution of tert-butyl 4-amino-4-methyl-piperidine-1-carboxylate (2 g, 9.33 mmol, 1 eq) 4-(methylamino)-2-methylsulfanyl-pyrimidine-5-carbaldehyde (1.71 g, 9.33 mmol, 1 eq) in MeOH (40 mL) was added AcOH (2.80 g, 46.66 mmol, 2.67 mL, 5 eq) and Ti(i-PrO)₄ (7.96 g, 28.00 mmol, 8.26 mL, 3 eq), the mixture was stirred at 60° C. for 12 hr and then NaBH₃CN (1.76 g, 28.00 mmol, 3 eq). The mixture was stirred at 60° C. for 2 hr. LC-MS showed desired compound was detected. The mixture was concentrated to get a residue, and the residue was quenched by Sat·NaHCO₃ 20 mL, the mixture was filtered and the filtrate was extracted by ethyl acetate (10 mL×3), the combined organic layers was dried by Na₂SO₄, filtered and the filtrate was concentrated to get a residue. The residue was purified by flash silica gel chromatography (12 g, Silica Flash Column, Eluent of 0-60% Ethyl acetate/Petroleum ether gradient @ 100 mL/min) to give compound tert-butyl 4-methyl-4-[[4-(methylamino)-2-methylsulfanyl-pyrimidin-5-yl]methylamino]piperidine-1-carboxylate (2.3 g, 6.03 mmol, 64.59% yield) as a colorless oil.

To a solution of tert-butyl 4-methyl-4-[[4-(methylamino)-2-methylsulfanyl-pyrimidin-5-yl]methylamino]piperidine-1-carboxylate (2.3 g, 6.03 mmol, 1 eq) and DIEA (3.90 g, 30.14 mmol, 5.25 mL, 5 eq) in DCM (20 mL) was added triphosgene (1.79 g, 6.03 mmol, 1 eq). The mixture was stirred at 20° C. for 12 hr. LC-MS showed desired compound was detected. The mixture was quenched by NaHCO₃ 20 mL, then the mixture was extracted by DCM (10 mL×3), the combined organic layers was washed by brine, then dried by Na₂SO₄, filtered and the filtrate was concentrated to get compound tert-butyl 4-methyl-4-(1-methyl-7-methylsulfanyl-2-oxo-4H-pyrimido[4,5-d]pyrimidin-S-yl)piperidine-1-carboxylate (2.3 g, crude) as an orange solid

To a solution of tert-butyl 4-methyl-4-(1-methyl-7-methylsulfanyl-2-oxo-4H-pyrimido[4,5-d]pyrimidin-S-yl)piperidine-1-carboxylate (1.3 g, 3.19 mmol, 1 eq) in DCM (10 mL) was added m-CPBA (1.17 g, 5.74 mmol, 85% purity, 1.8 eq). The mixture was stirred at 25° C. for 12 hr. LC-MS showed desired compound was detected. The reaction mixture was partitioned between sat. Na₂SO₃ 20 mL and dichloromethane 40 mL The organic phase was separated, washed with NaHCO₃ 20 mL dried over Na₂SO₄, filtered and concentrated under reduced pressure to give compound tert-butyl 4-methyl-4-(1-methyl-7-methylsulfonyl-2-oxo-4H-pyrimido[4,5-d]pyrimidin-S-yl)piperidine-1-carboxylate (1 g, crude) as a yellow solid.

To a solution of tert-butyl 4-methyl-4-(1-methyl-7-methylsulfonyl-2-oxo-4H-pyrimido[4,5-d]pyrimidin-S-yl)piperidine-1-carboxylate (220 mg, 500.54 umol, 1 eq) 2-(4-aminopyrazol-1-yl)ethanol (70.00 mg, 550.59 umol, 1.1 eq) in dioxane (3 mL) was added TFA (68.49 mg, 600.64 umol, 44.47 uL, 1.2 eq). The mixture was stirred at 80° C. for 12 hr. LC-MS showed desired compound was detected. The mixture was concentrated to get a residue. The residue was purified by flash silica gel chromatography (12 g Silica Flash Column, Eluent of 0-100% Ethyl acetate/Petroleum ether gradient @ 100 mL/min) to give compound tert-butyl 4-[7-[[1-(2-hydroxyethyl)pyrazol-4-yl]amino]-1-methyl-2-oxo-4H-pyrimido[4,5-d]pyrimidin-S-yl]-4-methyl-piperidine-1-carboxylate (330 mg, 678.22 umol, 67.75% yield) as a yellow oil.

To a solution of tert-butyl 4-[7-[[1-(2-hydroxyethyl)pyrazol-4-yl]amino]-1-methyl-2-oxo-4H-pyrimido[4,5-d]pyrimidin-S-yl]-4-methyl-piperidine-1-carboxylate (330 mg, 678.22 umol, 1 eq) in EtOAc (5 mL) was added HCl/EtOAc (5 mL, 4M). The mixture was stirred at 20° C. for 1 hr. The mixture was stirred at 25° C. for 1 hr. LC-MS showed desired compound was detected. The mixture was concentrated to give compound 7-[[1-(2-hydroxyethyl)pyrazol-4-yl]amino]-1-methyl-S-(4-methyl-4-piperidyl)-4H-pyrimido[4,5-d]pyrimidin-2-one (250 mg, crude, HCl) as a yellow solid.

To a solution of 7-[[1-(2-hydroxyethyl)pyrazol-4-yl]amino]-1-methyl-S-(4-methyl-4-piperidyl)-4H-pyrimido[4,5-d]pyrimidin-2-one (150 mg, 354.68 umol, 1 eq, HCl) and TEA (215.34 mg, 2.13 mmol, 296.20 uL, 6 eq) in DCM (3 mL) was added a solution of prop-2-enoyl chloride (32.10 mg, 354.68 umol, 28.92 uL, 1 eq) in DCM (0.5 mL). The mixture was stirred at 0° C. for 1 hr. LC-MS showed desired compound was detected. The solution was removed by a stream of N₂ to get a residue. The residue was purified by prep-HPLC (FA condition;column: Phenomenex luna C18 100×40 mm×3 um;mobile phase: [water(FA)-ACN];B %: 1%-40%, 8 min). Then purified by prep-HPLC (neutral condition column: Waters Xbridge BEH C18 100×30 mm×10 um;mobile phase: [water(NH4HCO₃)-ACN];B %: 1%-40%, 8 min) to give compound 7-[[1-(2-hydroxyethyl)pyrazol-4-yl]amino]-1-methyl-S-(4-methyl-1-prop-2-enoyl-4-piperidyl)-4H-pyrimido[4,5-d]pyrimidin-2-one (13.1 mg, 29.74 umol, 8.38% yield, 100% purity) as a white solid.

### Procedure for Preparation of Compound 121

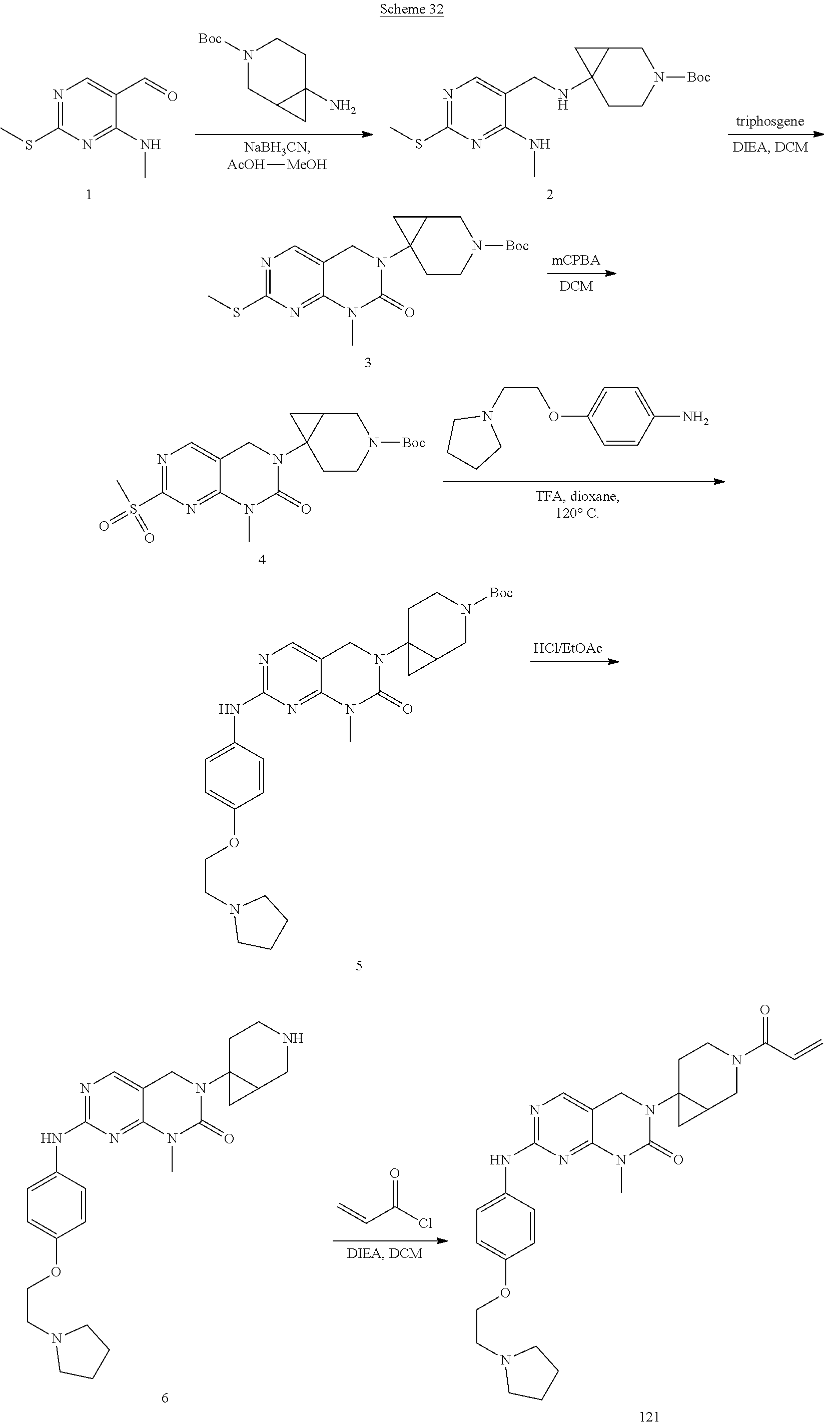

To a solution of tert-butyl 6-amino-S-azabicyclo[4.1.0]heptane-S-carboxylate(500 mg, 2.4 mmol, 1 eq) in MeOH (30 mL) was added 4-(methylamino)-2-methylsulfanyl-pyrimidine-5-carbaldehyde (431.6 mg, 2.4 mmol, 1 eq), Ti(i-PrO)₄ (1.0 g, 3.5 mmol, 1.0 mL, 1.5 eq) and AcOH (848.6 mg, 14.1 mmol, 808.2 uL, 6 eq) was added to the mixture to adjust pH=5 and the mixture was stirred at 25° C. for 2 hr. NaBH₃CN (444.0 mg, 7.0 mmol, 3 eq) was added to the mixture. The mixture was stirred at 65° C. for 12 hr. LC-MS showed desired mass was detected. The reaction mixture was concentrated under reduced pressure to remove solvent. The residue was diluted with NaHCO₃ (30 mL) and extracted with dichloromethane (30 mL×3). The combined organic layers were washed with brine (30 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 100 mL/min) to yield compound tert-butyl 6-[[4-(methylamino)-2-methylsulfanyl-pyrimidin-5-yl]methylamino]-S-azabicyclo[4.1.0]heptane-S-carboxylate (700 mg, 1.8 mmol, 78.3% yield) obtained as a yellow solid.

To a solution of tert-butyl 6-[[4-(methylamino)-2-methylsulfanyl-pyrimidin-5-yl]methylamino]-S-azabicyclo[4.1.0]heptane-S-carboxylate (540 mg, 1.4 mmol, 1 eq) in DCM (5 mL) was added DIEA (551.7 mg, 4.3 mmol, 743.5 uL, 3 eq) and bis(trichloromethyl) carbonate (337.8 mg, 1.1 mmol, 0.8 eq) at 0° C. The mixture was stirred at 25° C. for 12 hr. The reaction was monitored by LCMS which showed the reactant was consumed completely and the desired mass peak was detected. The residue was diluted with NaHCO₃ 30 mL and extracted with dichloromethane (50 mL×3). The combined organic layers were washed with brine (50 mL×1), dried over Na₂SO₄, filtered and concentrated under reduced pressure to give compound tert-butyl 6-(1-methyl-7-methylsulfanyl-2-oxo-4H-pyrimido[4,5-d]pyrimidin-3-yl)-S-azabicyclo[4.1.0]heptane-S-carboxylate (700 mg, crude) obtained as yellow oil.

To a solution of tert-butyl 6-(1-methyl-7-methylsulfanyl-2-oxo-4H-pyrimido[4,5-d]pyrimidin-S-yl)-S-azabicyclo[4.1.0]heptane-S-carboxylate (600 mg, 1.5 mmol, 1 eq) in DCM (8 mL) was added m-CPBA (540.7 mg, 2.7 mmol, 85% purity, 1.8 eq) at 0° C. The mixture was stirred at 25° C. for 1.5 hr. The reaction was monitored by LCMS which showed complete consumption of reactant and detection of the desired mass peak. The residue was diluted with Na₂SO₃ (10%) (10 mL). and extracted with Dichloromethane (20 mL×3). The combined organic layers were washed with NaHCO₃ (20 mL×3), dried over Na₂SO₄, filtered and concentrated under reduced pressure to give compound tert-butyl 6-(1-methyl-7-methylsulfonyl-2-oxo-4H-pyrimido[4,5-d]pyrimidin-S-yl)-S-azabicyclo[4.1.0]heptane-S-carboxylate (800 mg, crude) obtained as a white solid.

tert-butyl 6-(1-methyl-7-methylsulfonyl-2-oxo-4H-pyrimido[4,5-d]pyrimidin-S-yl)-S-azabicyclo[4.1.0]heptane-S-carboxylate (180 mg, 411.4 umol, 1 eq) in dioxane (2 mL) was added 4-(2-pyrrolidin-1-ylethoxy)aniline (93.4 mg, 452.6 umol, 1.1 eq) and TFA (70.4 mg, 617.1 umol, 45.7 uL, 1.5 eq). The reaction mixture was split in 4 batches in parallel and stirred at 120° C. for 12 hr. The reaction was monitored by LCMS which showed complete consumption of reactant and detection of the desired mass peak. The combined reaction mixture was concentrated under reduced pressure to give a residue. The residue was purified by prep-HPLC (FA condition; column: Phenomenex Luna C18 200×40 mm×10 um;mobile phase: [water(FA)-ACN];B %: 10%-45%, 8 min). to yield compound tert-butyl 6-[1-methyl-2-oxo-7-[4-(2-pyrrolidin-1-ylethoxy)anilino]-4H-pyrimido[4,5-d]pyrimidin-3-yl]-S-azabicyclo[4.1.0]heptane-S-carboxylate (300 mg, 532.2 umol, 32.4% yield) obtained as a yellow solid.

A solution of tert-butyl 6-[1-methyl-2-oxo-7-[4-(2-pyrrolidin-1-ylethoxy)anilino]-4H-pyrimido[4,5-d]pyrimidin-S-yl]-S-azabicyclo[4.1.0]heptane-S-carboxylate (100 mg, 177.4 umol, 1 eq) in HCl/EtOAc (2 mL) (4 M) was stirred at 25° C. for 10 min. The reaction was monitored by LCMS which showed complete consumption of reactant and detection of the desired product. The reaction mixture was concentrated under reduced pressure to give the crude product 3-(3-azabicyclo[4.1.0]heptan-6-yl)-1-methyl-7-[4-(2-pyrrolidin-1-ylethoxy)anilino]-4H-pyrimido[4,5-d]pyrimidin-2-one (250 mg, crude) obtained as yellow solid used into the next step without further purification.

To a solution of 3-(3-azabicyclo[4.1.0]heptan-6-yl)-1-methyl-7-[4-(2-pyrrolidin-1-ylethoxy)anilino]-4H-pyrimido[4,5-d]pyrimidin-2-one (30 mg, 64.7 umol, 1 eq) and DIEA (25.1 mg, 194.1 umol, 33.8 uL, 3 eq) in DCM (2 mL) was added prop-2-enoyl chloride (7.0 mg, 77.7 umol, 6.3 uL, 1.2 eq) at 0° C. The mixture was stirred at 25° C. for 1 hr. The reaction was monitored by LCMS which showed complete consumption of reactant and detection of the desired mass peak. The reaction mixture was concentrated under reduced pressure to remove solvent. The residue was diluted with DMF (3 mL) and purified by prep-HPLC (FA condition; column: Phenomenex Luna C18 200×40 mm×10 um;mobile phase: [water(FA)-ACN];B %: 1%-35%, 8 min) to yield compound 1-methyl-S-(3-prop-2-enoyl-S-azabicyclo[4.1.0]heptan-6-yl)-7-[4-(2-pyrrolidin-1-ylethoxy)anilino]-4H-pyrimido[4,5-d]pyrimidin-2-one (12 mg, 20.9 umol, 16.2% yield, 98.30% purity, FA) obtained as a white solid.

### Procedure for Preparation of Compound 129

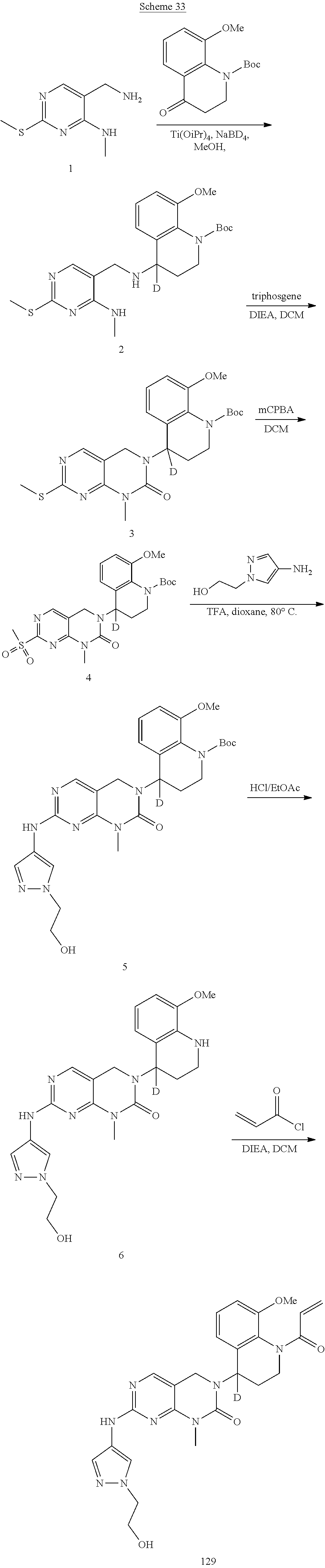

To a solution of 5-(aminomethyl)-N-methyl-2-methylsulfanyl-pyrimidin-4-amine (498.3 mg, 2.3 mmol, 8.4 e-1 eq, HCl) in MeOH (15 mL) was added Ti(i-PrO)₄ (2.3 g, 8.1 mmol, 2.4 mL, 3 eq), AcOH (974.4 mg, 16.2 mmol, 928.0 uL, 6 eq) and tert-butyl 8-methoxy-4-oxo-2,3-dihydroquinoline-1-carboxylate (750 mg, 2.7 mmol, 1 eq). The mixture was stirred at 65° C. for 12 hr. Then sodium tetradeuterioboranuide (307.0 mg, 8.1 mmol, 3 eq) was added to the mixture at 0° C. The mixture was stirred at 20° C. for 3 hr. LC-MS showed desired mass was detected. The reaction mixture was concentrated under reduced pressure to remove solvent. The residue was diluted with NH₄Cl (50 mL) and extracted with ethyl acetate (50 mL×3). The combined organic layers were washed with NaCl (50 mL×3), dried over Na₂SO₄, filtered and concentrated under reduced pressure to give a residue. The residue was purified by flash silica gel chromatography (40 g Silica Flash Column, Eluent of 0-100% Ethyl acetate/Petroleum ether gradient @ 120 mL/min) to yield compound tert-butyl 4-deuterio-8-methoxy-4-[[4-(methylamino)-2-methylsulfanyl-pyrimidin-5-yl]methylamino]-2,3-dihydroquinoline-1-carboxylate (1.2 g, 2.7 mmol, 24.8% yield) obtained as a yellow solid.

To a solution of tert-butyl 4-deuterio-8-methoxy-4-[[4-(methylamino)-2-methylsulfanyl-pyrimidin-5-yl]methylamino]-2,3-dihydroquinoline-1-carboxylate (1.4 g, 3.1 mmol, 1 eq) in DCM (20 mL) was added triphosgene (744.2 mg, 2.5 mmol, 0.8 eq) and DIEA(1.2 g, 9.4 mmol, 1.6 mL, 3 eq) at 0° C. The mixture was stirred at 25° C. for 12 hr. The reaction was monitored by LCMS which showed the reactant was consumed completely and the desired mass peak was detected. The residue was diluted with NaHCO₃ (30 mL) and extracted with dichloromethane (40 mL×3). The combined organic layers were washed with brine (40 mL×1), dried over Na₂SO₄, filtered and concentrated under reduced pressure to give the crude product tert-butyl 4-deuterio-8-methoxy-4-(1-methyl-7-methylsulfanyl-2-oxo-4H-pyrimido[4,5-d]pyrimidin-S-yl)-2,3-dihydroquinoline-1-carboxylate (2.5 g, crude) obtained as yellow solid used into the next step without further purification.

To a solution of tert-butyl 4-deuterio-8-methoxy-4-(1-methyl-7-methylsulfanyl-2-oxo-4H-pyrimido[4,5-d]pyrimidin-S-yl)-2,3-dihydroquinoline-1-carboxylate (1.5 g, 3.1 mmol, 1 eq) in DCM (15 mL) was added m-CPBA (953.7 mg, 4.7 mmol, 85% purity, 1.5 eq). The mixture was stirred at 0° C. for 2 hr. The reaction was monitored by LCMS which showed complete consumption of reactant and detection of the desired mass peak. The reaction mixture was concentrated under reduced pressure to remove solvent. The residue was diluted with Na₂SO₃ (60 mL) and extracted with dichloromethane (60 mL×3). The combined organic layers were washed with NaHCO₃ (60 mL×2), dried over Na₂SO₄, filtered and concentrated under reduced pressure to give compound tert-butyl 4-deuterio-8-methoxy-4-(1-methyl-7-methylsulfonyl-2-oxo-4H-pyrimido[4,5-d]pyrimidin-S-yl)-2,3-dihydroquinoline-1-carboxylate (1.4 g, crude) obtained as a yellow solid.

To a solution of tert-butyl 4-deuterio-8-methoxy-4-(1-methyl-7-methylsulfonyl-2-oxo-4H-pyrimido[4,5-d]pyrimidin-S-yl)-2,3-dihydroquinoline-1-carboxylate (200 mg, 396.4 umol, 1 eq) in dioxane (0.7 mL) was added TFA (45.2 mg, 396.4 umol, 29.4 uL, 1 eq) and 2-(4-aminopyrazol-1-yl)ethanol (60.5 mg, 475.7 umol, 1.2 eq). The mixture was stirred at 80° C. for 12 hr(6×batch). The reaction was monitored by LCMS which showed complete consumption of reactant and detection of the desired mass peak. The reaction mixture was concentrated under reduced pressure to give a residue. The residue was purified by prep-HPLC (FA condition, column: Phenomenex Luna C18 200×40 mm×10 um;mobile phase: [water(FA)-ACN];B %: 15%-55%, 8 min) to yield compound tert-butyl 4-deuterio-4-[7-[[1-(2-hydroxyethyl)pyrazol-4-yl]amino]-1-methyl-2-oxo-4H-pyrimido[4,5-d]pyrimidin-S-yl]-8-methoxy-2,3-dihydroquinoline-1-carboxylate (130 mg, 235.7 umol, 29.7% yield) obtained as a yellow solid.

To a solution of tert-butyl 4-deuterio-4-[7-[[1-(2-hydroxyethyl)pyrazol-4-yl]amino]-1-methyl-2-oxo-4H-pyrimido[4,5-d]pyrimidin-S-yl]-8-methoxy-2,3-dihydroquinoline-1-carboxylate (130 mg, 235.7 umol, 1 eq) in HCl/EtOAc (1 mL, 4 M). The mixture was stirred at 25° C. for 3 hr. The reaction was monitored by LCMS which showed complete consumption of reactant and detection of the desired product. The reaction mixture was concentrated under reduced pressure to give compound 3-(4-deuterio-8-methoxy-2,3-dihydro-1 H-quinolin-4-yl)-7-[[1-(2-hydroxyethyl)pyrazol-4-yl]amino]-1-methyl-4H-pyrimido[4,5-d]pyrimidin-2-one (130 mg, crude, HCl) obtained as a brown solid.

To a solution of 3-(4-deuterio-8-methoxy-2,3-dihydro-1 H-quinolin-4-yl)-7-[[1-(2-hydroxyethyl)pyrazol-4-yl]amino]-1-methyl-4H-pyrimido[4,5-d]pyrimidin-2-one (50 mg, 102.5 umol, 1 eq, HCl) and DIEA (39.7 mg, 307.4 umol, 53.5 uL, 3 eq) in DCM (2 mL) was added prop-2-enoyl chloride (4.6 mg, 51.2 umol, 4.2 uL, 0.5 eq) at 0° C. The mixture was stirred at 25° C. for 1 hr. The reaction was monitored by LCMS which showed complete consumption of reactant and detection of the desired mass peak. The reaction mixture was concentrated under reduced pressure to remove solvent. The residue was purified by prep-HPLC (FA condition, column: Phenomenex Luna C18 200×40 mm×10 um;mobile phase: [water(FA)-ACN];B %: 10%-40%, 8 min to yield compound 3-(4-deuterio-8-methoxy-1-prop-2-enoyl-2,3-dihydroquinolin-4-yl)-7-[[1-(2-hydroxyethyl)pyrazol-4-yl]amino]-1-methyl-4H-pyrimido[4,5-d]pyrimidin-2-one (2 mg, 4.00 umol, 3.9% yield) as a white solid.

### Procedure for Preparation of Compound 132

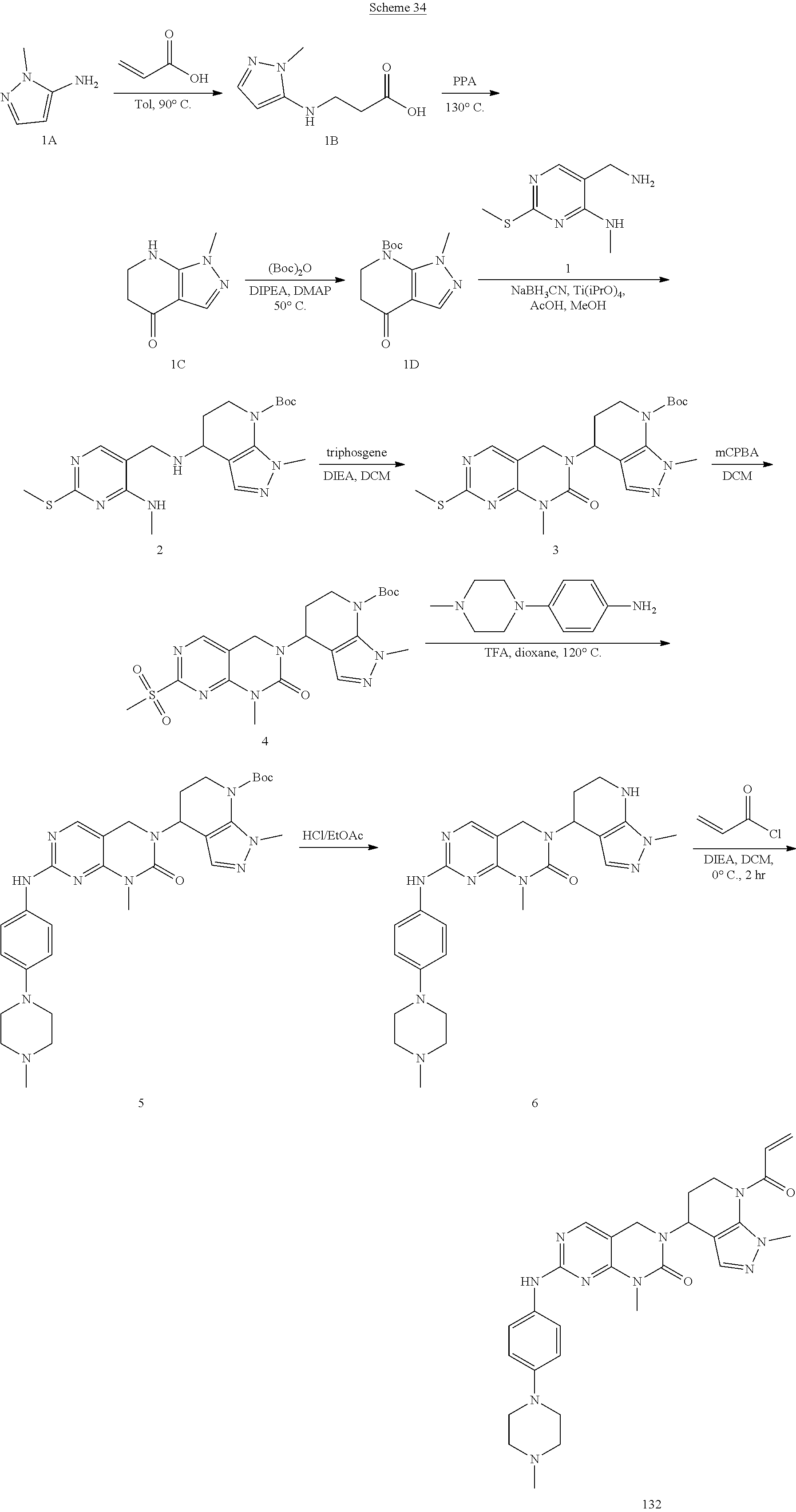

To a solution of 2-methylpyrazol-S-amine (10 g, 103.0 mmol, 1 eq) in Tol. (150 mL) was added acrylic acid (7.4 g, 103.0 mmol, 7.1 mL, 1 eq). The mixture was stirred at 90° C. for 24 hr. LC-MS showed desired compound was detected. The mixture was concentrated to get a residue. The crude product was triturated with EtOAc/MeOH=10/1 (120 mL) at 25° C. for 12 hr to give compound 3-[(2-methylpyrazol-S-yl)amino]propanoic acid (30 g, crude) as a white solid.

3-[(2-methylpyrazol-S-yl)amino]propanoic acid (15 g, 88.7 mmol, 1 eq) was dissolved in PPA (150 mL), the mixture was stirred at 130° C. for 12 hr. LC-MS showed desired compound was detected. The hot mixture was poured into an ice NaOH (50%, aq) solution 500 mL, then the mixture was cooled to 25° C. then extracted by ethyl acetate (100 mL×3) the organic layers was washed by brine (100 mL×3), then dried by Na₂SO₄, filtered and the filtrate was concentrated to get crude product 1-methyl-6,7-dihydro-5H-pyrazolo[3,4-b]pyridin-4-one (6 g, crude) as a red oil.

To a solution of 1-methyl-6,7-dihydro-5H-pyrazolo[3,4-b]pyridin-4-one (5 g, 33.1 mmol, 1 eq) DMAP (404.1 mg, 3.3 mmol, 0.1 eq) in DCM (10 mL) was added Boc₂O (8.7 g, 39.7 mmol, 9.12 mL, 1.2 eq) and DIPEA (12.8 g, 99.2 mmol, 17.28 mL, 3 eq). The mixture was stirred at 25° C. for 12 hr. LC-MS showed desired compound was detected. The mixture was concentrated to get a residue. The residue was purified by column chromatography (SiO₂, Petroleum ether/Ethyl acetate=10/1 to 3/1) to give compound tert-butyl 1-methyl-4-oxo-5,6-dihydropyrazolo[3,4-b]pyridine-7-carboxylate (2.4 g, 9.6 mmol, 28.88% yield) as an orange oil.

To a solution of 5-(aminomethyl)-N-methyl-2-methylsulfanyl-pyrimidin-4-amine (1.76 g, 9.6 mmol, 1 eq) and tert-butyl 1-methyl-4-oxo-5,6-dihydropyrazolo[3,4-b]pyridine-7-carboxylate (2.4 g, 9.6 mmol, 1 eq) in MeOH (30 mL) was added AcOH (2.9 g, 47.8 mmol, 2.73 mL, 5 eq) and Ti(i-PrO)₄ (8.1 g, 28.7 mmol, 8.5 mL, 3 eq), the mixture was stirred at 25° C. for 12 hr. Then NaBH₃CN (1.80 g, 28.7 mmol, 3 eq) was added, the reaction was stirred at 25° C. for 2 hr. LC-MS showed desired compound was detected. The reaction mixture was filtered and the filtrate was quenched by Sat. NaHCO₃ 100 mL, then the mixture was extracted by ethyl acetate (50 mL×3), the combined organic layers was dried by Na₂SO₄, filtered and the filtrate was concentrated to get a residue. The residue was purified by column chromatography (SiO₂, Petroleum ether/Ethyl acetate=10/1 to 1/1) to give compound tert-butyl 1-methyl-4-[[4-(methylamino)-2-methylsulfanyl-pyrimidin-5-yl]methylamino]-5,6-dihydro-4H-pyrazolo[3,4-b]pyridine-7-carboxylate (460 mg, 1.1 mmol, 11.48% yield) as a yellow oil.

To a solution of tert-butyl 1-methyl-4-[[4-(methylamino)-2-methylsulfanyl-pyrimidin-5-yl]methylamino]-5,6-dihydro-4H-pyrazolo[3,4-b]pyridine-7-carboxylate (460 mg, 1.10 mmol, 1 eq) in DCM (10 mL) was added triphosgene (195.2 mg, 657.9 umol, 0.6 eq) and DIEA (425.1 mg, 3.3 mmol, 572.9 uL, 3 eq). The mixture was stirred at 25° C. for 1 hr. LC-MS showed desired compound was detected. The mixture was concentrated to get a residue, and the residue was quenched by Sat. NH₄Cl 20 mL and extracted by ethyl acetate (20 mL×3), the organic layers was washed by brine (10 mL×2) and dried by Na₂SO₄, filtered and the filtrate was concentrated to get a residue. The residue was purified by column chromatography (SiO₂, Petroleum ether/Ethyl acetate=3/1 to 0/1) to give compound tert-butyl 4-[[6-chloro-4-(methylamino)pyridazin-S-yl]methylamino]-3,4-dihydro-2H-quinoline-1-carboxylate (1.1 g, 2.7 mmol, 49.1% yield) as an orange oil.

To a solution of tert-butyl 1-methyl-4-(1-methyl-7-methylsulfanyl-2-oxo-4H-pyrimido[4,5-d]pyrimidin-S-yl)-5,6-dihydro-4H-pyrazolo[3,4-b]pyridine-7-carboxylate (420 mg, 942.7 umol, 1 eq) in DCM (10 mL) was added m-CPBA (306.2 mg, 1.5 mmol, 85% purity, 1.6 eq). The mixture was stirred at 25° C. for 1 hr. LC-MS showed desired compound was detected. The mixture was quenched by sat·NaHCO₃ 15 mL and then extracted by DCM (15 mL×3), the combined organic layers was washed by brine and dried by Na₂SO₄, filtered and the filtrate was concentrated to get a residue. The residue was purified by column chromatography (SiO₂, Petroleum ether/Ethyl acetate=10/1 to 1/1) to give compound tert-butyl 1-methyl-4-(1-methyl-7-methylsulfanyl-2-oxo-4H-pyrimido[4,5-d]pyrimidin-S-yl)-5,6-dihydro-4H-pyrazolo[3,4-b]pyridine-7-carboxylate (420 mg, 942.7 umol, 85.98% yield) as a yellow oil

To a solution of tert-butyl 1-methyl-4-(1-methyl-7-methylsulfinyl-2-oxo-4H-pyrimido[4,5-d]pyrimidin-S-yl)-5,6-dihydro-4H-pyrazolo[3,4-b]pyridine-7-carboxylate (180 mg, 390.0 umol, 1 eq) in dioxane (2 mL) was added TFA (53.36 mg, 468.00 umol, 34.7 uL, 1.2 eq) and 4-(4-methylpiperazin-1-yl)aniline (82.1 mg, 429.0 umol, 1.1 eq). The mixture was stirred at 120° C. for 12 hr. LC-MS showed desired compound was detected. The mixture was concentrated to get a residue. The residue was purified by prep-HPLC (TFA condition column: Phenomenex Luna C18 100×30 mm×5 um;mobile phase: [water(TFA)-ACN];B %: 5%-35%, 8 min) to give compound tert-butyl 1-methyl-4-[1-methyl-7-[4-(4-methylpiperazin-1-yl)anilino]-2-oxo-4H-pyrimido[4,5-d]pyrimidin-S-yl]-5,6-dihydro-4H-pyrazolo[3,4-b]pyridine-7-carboxylate (120 mg, 203.8 umol, 52.27% yield) as a brown solid.

To a solution of tert-butyl 1-methyl-4-[1-methyl-7-[4-(4-methylpiperazin-1-yl)anilino]-2-oxo-4H-pyrimido[4,5-d]pyrimidin-S-yl]-5,6-dihydro-4H-pyrazolo[3,4-b]pyridine-7-carboxylate (120 mg, 203.8 umol, 1 eq) in EtOAc (1 mL) was added HCl/EtOAc (1 mL, 4M). The mixture was stirred at 25° C. for 1 hr. LC-MS showed desired compound was detected. the mixture was concentrated to give compound 1-methyl-7-[4-(4-methylpiperazin-1-yl)anilino]-S-(1-methyl-4,5,6,7-tetrahydropyrazolo[3,4-b]pyridin-4-yl)-4H-pyrimido[4,5-d]pyrimidin-2-one (120 mg, crude, HCl) as a brown solid.

To a solution of 1-methyl-7-[4-(4-methylpiperazin-1-yl)anilino]-S-(1-methyl-4,5,6,7-tetrahydropyrazolo[3,4-b]pyridin-4-yl)-4H-pyrimido[4,5-d]pyrimidin-2-one (60 mg, 114.3 umol, 1 eq, HCl) and DIEA (73.9 mg, 571.4 umol, 99.5 uL, 5 eq) in DCM (2 mL) was added prop-2-enoyl chloride (10.34 mg, 114.3 umol, 9.3 uL, 1 eq) at 0° C. The mixture was stirred at 25° C. for 1 hr. LC-MS showed desired compound was detected. The mixture was concentrated to get a residue. The residue was purified by prep-HPLC (FA condition; column: Phenomenex Luna C18 200×40 mm×10 um;mobile phase: [water(FA)-ACN];B %: 1%-25%, 8 min) to give compound 1-methyl-7-[4-(4-methylpiperazin-1-yl)anilino]-S-(1-methyl-7-prop-2-enoyl-5,6-dihydro-4H-pyrazolo[3,4-b]pyridin-4-yl)-4H-pyrimido[4,5-d]pyrimidin-2-one (17 mg, 30.44 umol, 13.32% yield, 99.64% purity, 0.3FA) was obtained as a white solid.

### Procedure for Preparation of Compound 130

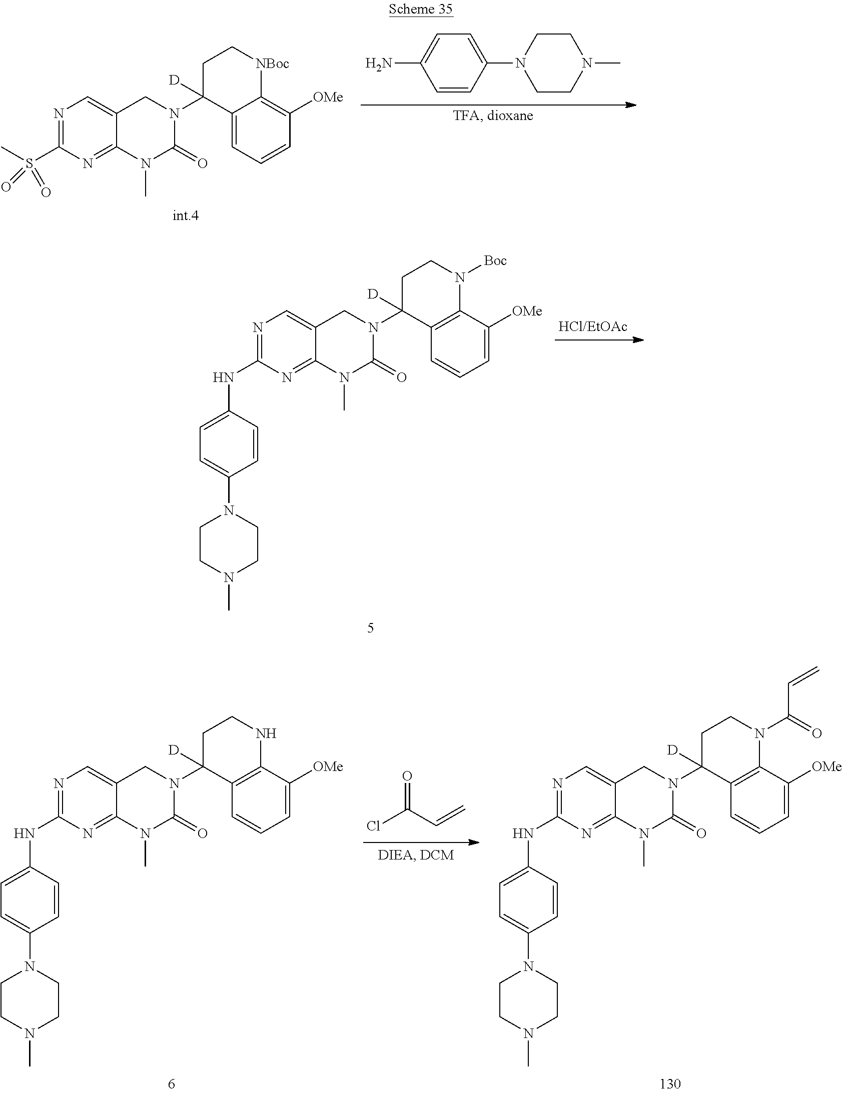

To a solution of tert-butyl 4-deuterio-8-methoxy-4-(1-methyl-7-methylsulfonyl-2-oxo-4H-pyrimido[4,5-d]pyrimidin-S-yl)-2,3-dihydroquinoline-1-carboxylate (200 mg, 396.4 umol, 1 eq) in dioxane (2.5 mL) was added TFA (45.2 mg, 396.4 umol, 29.4 uL, 1 eq) and 4-(4-methylpiperazin-1-yl)aniline (91.00 mg, 475.7 umol, 1.2 eq). The reaction solution was spitted in 4 batches in parallel and the mixture was stirred at 80° C. for 12 hr. LCMS showed consumption of reactant and formation of the desired product mass. The reaction mixture was combined and concentrated under reduced pressure to give a residue. The residue was purified by prep-HPLC (FA condition; column: Phenomenex Luna C18 200×40 mm×10 um; mobile phase: [water(FA)-ACN];B %: 15%-55%, 8 min) to yield compound tert-butyl 4-deuterio-8-methoxy-4-[1-methyl-7-[4-(4-methylpiperazin-1-yl)anilino]-2-oxo-4H-pyrimido[4,5-d]pyrimidin-S-yl]-2,3-dihydroquinoline-1-carboxylate (180 mg, 292.3 umol, 73.8% yield) obtained as a brown solid.

A solution of tert-butyl 4-deuterio-8-methoxy-4-[1-methyl-7-[4-(4-methylpiperazin-1-yl)anilino]-2-oxo-4H-pyrimido[4,5-d]pyrimidin-S-yl]-2,3-dihydroquinoline-1-carboxylate (90 mg, 146.2 umol, 1 eq) in HCl/EtOAc (4 mL, 4 M) was stirred at 25° C. for 10 min. The reaction was monitored by LCMS which showed the reactant was consumed completely and the desired mass peak was detected. The reaction mixture was concentrated under reduced pressure to give the crude product 3-(4-deuterio-8-methoxy-2,3-dihydro-1H-quinolin-4-yl)-1-methyl-7-[4-(4-methylpiperazin-1-yl)anilino]-4H-pyrimido[4,5-d]pyrimidin-2-one (200 mg, crude) obtained as yellow solid used into the next step without further purification.

To a solution of 3-(4-deuterio-8-methoxy-2,3-dihydro-1H-quinolin-4-yl)-1-methyl-7-[4-(4-methylpiperazin-1-yl)anilino]-4H-pyrimido[4,5-d]pyrimidin-2-one (50 mg, 90.6 umol, 1 eq, HCl) and DIEA (35.1 mg, 271.7 umol, 47.3 uL, 3 eq) in DCM (2 mL) was added prop-2-enoyl chloride (8.2 mg, 90.6 umol, 7.4 uL, 1 eq) at 0° C. The mixture was stirred at 25° C. for 1 hr. The reaction was monitored by LCMS which showed complete consumption of reactant and detection of the desired mass peak. The reaction mixture was concentrated under reduced pressure to remove solvent. The residue was diluted with DMF (3 mL) and purified by prep-HPLC (FA condition; column: Phenomenex Luna C18 200×40 mm×10 um; mobile phase: [water (FA)-ACN]; B %: 15%-50%, 8 min). The residue was further purified by prep-HPLC neutral condition column: Waters Xbridge Prep OBD C18 150×40 mm×10 um;mobile phase: [water(NH4HCO3)-ACN];B %: 25%-65%, 8 min) to yield 3-(4-deuterio-8-methoxy-1-prop-2-enoyl-2,3-dihydroquinolin-4-yl)-1-methyl-7-[4-(4-methylpiperazin-1-yl)anilino]-4H-pyrimido[4,5-d]pyrimidin-2-one (5 mg, 8.8 umol, 4.9% yield, 100% purity) obtained as a white solid.

### Procedure for Preparation of Compound 133

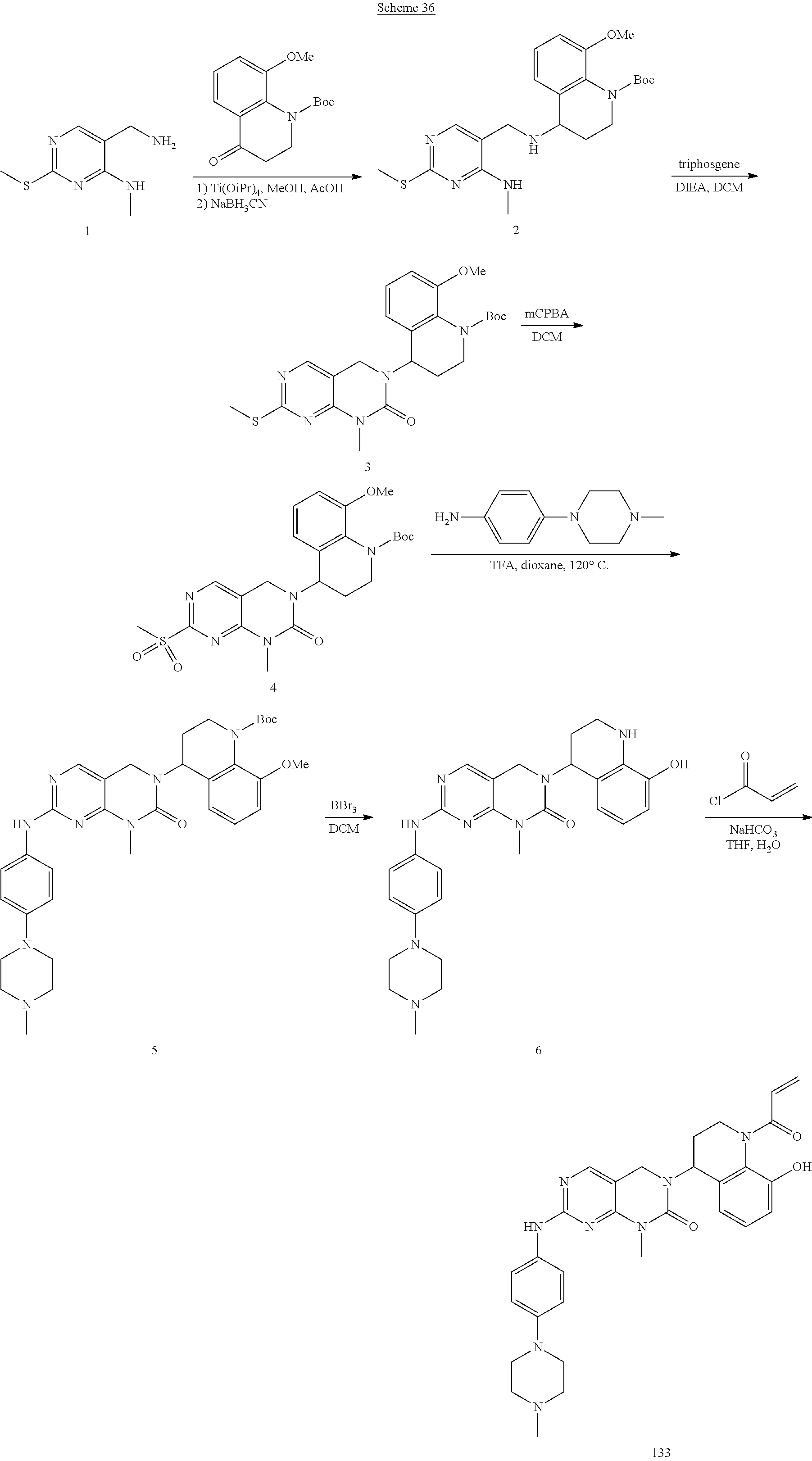

To a solution of 5-(aminomethyl)-N-methyl-2-methylsulfanyl-pyrimidin-4-amine (664.45 mg, 3.61 mmol, 1 eq) in MeOH (20 mL) was added Ti(i-PrO)₄ (1.5 g, 5.4 mmol, 1.6 mL, 1.5 eq), tert-butyl 8-methoxy-4-oxo-2,3-dihydroquinoline-1-carboxylate (1 g, 3.61 mmol, 1 eq) and CH₃COOH (1.3 g, 21.6 mmol, 1.2 mL, 6 eq). The mixture was stirred at 65° C. for 12 hr. Then NaBH₃CN (679.8 mg, 10.8 mmol, 3 eq) was added to the mixture. The mixture was stirred at 65° C. for 12 hr. LC-MS showed desired compound was detected. The reaction mixture was concentrated under reduced pressure to remove solvent. The residue was diluted with NaHCO₃ 200 mL and extracted with ethyl acetate (200 mL×3). The combined organic layers were washed with aqueous NaCl (100 mL×1), dried over Na₂SO₄, filtered and concentrated under reduced pressure to give a residue. The residue was purified by flash silica gel chromatography (20 g Silica Flash Column, Eluent of 0-50% Ethyl acetate/Petroleum ether gradient @ 120 mL/min) to give tert-butyl 8-methoxy-4-[[4-(methylamino)-2-methylsulfanyl-pyrimidin-5-yl]methylamino]-3,4-dihydro-2H-quinoline-1-carboxylate (200 mg, 448.8 umol, 12.5% yield) as a yellow oil.

To a solution of tert-butyl 8-methoxy-4-[[4-(methylamino)-2-methylsulfanyl-pyrimidin-5-yl]methylamino]-3,4-dihydro-2H-quinoline-1-carboxylate (200 mg, 448.9 umol, 1 eq) in DCM (5 mL) was added DIEA (227.1 mg, 2.2 mmol, 312.4 uL, 5 eq) and triphosgene (133.2 mg, 448.9 umol, 1 eq) at 0° C. The mixture was stirred at 0° C. for 2 hr. LC-MS showed desired compound was detected. The residue was diluted with NaHCO₃ 50 mL and extracted with ethyl acetate (50 mL×3). The combined organic layers were washed with brine (30 mL×1), dried over Na₂SO₄, filtered and concentrated under reduced pressure to give tert-butyl 8-methoxy-4-(1-methyl-7-methylsulfanyl-2-oxo-4H-pyrimido[4,5-d]pyrimidin-S-yl)-3,4-dihydro-2H-quinoline-1-carboxylate (247 mg, crude) as a yellow oil.

To a solution of tert-butyl 8-methoxy-4-(1-methyl-7-methylsulfanyl-2-oxo-4H-pyrimido[4,5-d]pyrimidin-S-yl)-3,4-dihydro-2H-quinoline-1-carboxylate (247 mg, 523.8 umol, 1 eq) in DCM (3 mL) was added m-CPBA (159.5 mg, 785.7 umol, 85% purity, 1.5 eq) at 0° C.

The mixture was stirred at 25° C. for 2 hr. LC-MS showed desired compound was detected.

The reaction mixture was partitioned between Na₂SO₃ 30 mL and dichloromethane (30×3 mL). The organic phase was separated, washed with NaHCO₃ (20 mL×3), dried over Na₂SO₄, filtered and concentrated under reduced pressure to give tert-butyl 8-methoxy-4-(1-methyl-7-methylsulfonyl-2-oxo-4H-pyrimido[4,5-d]pyrimidin-S-yl)-3,4-dihydro-2H-quinoline-1-carboxylate (220 mg, crude) as a yellow solid.

To a solution of tert-butyl 8-methoxy-4-(1-methyl-7-methylsulfonyl-2-oxo-4H-pyrimido[4,5-d]pyrimidin-S-yl)-3,4-dihydro-2H-quinoline-1-carboxylate (355 mg, 705.0 umol, 1 eq) in dioxane (1 mL) was added 4-(4-methylpiperazin-1-yl)aniline (161.8 mg, 846.0 umol, 1.2 eq) and TFA (120.6 mg, 1.1 mmol, 78.3 uL, 1.5 eq). The mixture was stirred at 120° C. for 12 hr. LC-MS showed desired compound was detected. The reaction mixture was concentrated under reduced pressure to remove solvent. The residue was purified by prep-HPLC (TFA condition) column: Phenomenex Luna C18 200×40 mm×10 um; mobile phase: [water(TFA)-ACN]; B %: 15%-45%, 8 min to give tert-butyl 8-methoxy-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (240 mg, 390.4 umol, 55.4% yield) as a yellow solid.

To a solution of tert-butyl 8-methoxy-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (220 mg, 357.9 umol, 1 eq) in DCM (2 mL) was added BBr₃ (448.3 mg, 1.8 mmol, 172.4 uL, 5 eq) at 0° C. The mixture was stirred at 25° C. for 12 hr. LC-MS showed desired compound was detected. The residue was poured into ice-water (2 mL). The mixture was diluted with H₂O 5 mL and extracted with (Dichloromethane: Methanol=10:1) (10 mL×3), dried over Na₂SO₄, filtered and concentrated under reduced pressure to give 3-(8-hydroxy-1,2,3,4-tetrahydroquinolin-4-yl)-1-methyl-7-[4-(4-methylpiperazin-1-yl)anilino]-4H-pyrimido[4,5-d]pyrimidin-2-one (120 mg, crude) as a yellow solid.

To a solution of 3-(8-hydroxy-1,2,3,4-tetrahydroquinolin-4-yl)-1-methyl-7-[4-(4-methylpiperazin-1-yl)anilino]-4H-pyrimido[4,5-d]pyrimidin-2-one (50 mg, 99.9 umol, 1 eq) and NaHCO₃ (25.2 mg, 299.6 umol, 11.6 uL, 3 eq) in THE (1 mL)/H₂O (1 mL) was added prop-2-enoyl chloride (9.0 mg, 99.9 umol, 8.1 uL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 1%-35%, 8 min to give 3-(8-hydroxy-1-prop-2-enoyl-3,4-dihydro-2H-quinolin-4-yl)-1-methyl-7-[4-(4-methylpiperazin-1-yl)anilino]-4H-pyrimido[4,5-d]pyrimidin-2-one (16.5 mg, 27.4 umol, 27.4% yield, 99.7% purity, FA) was obtained as a white solid.

### Procedure for Preparation of Compound 131

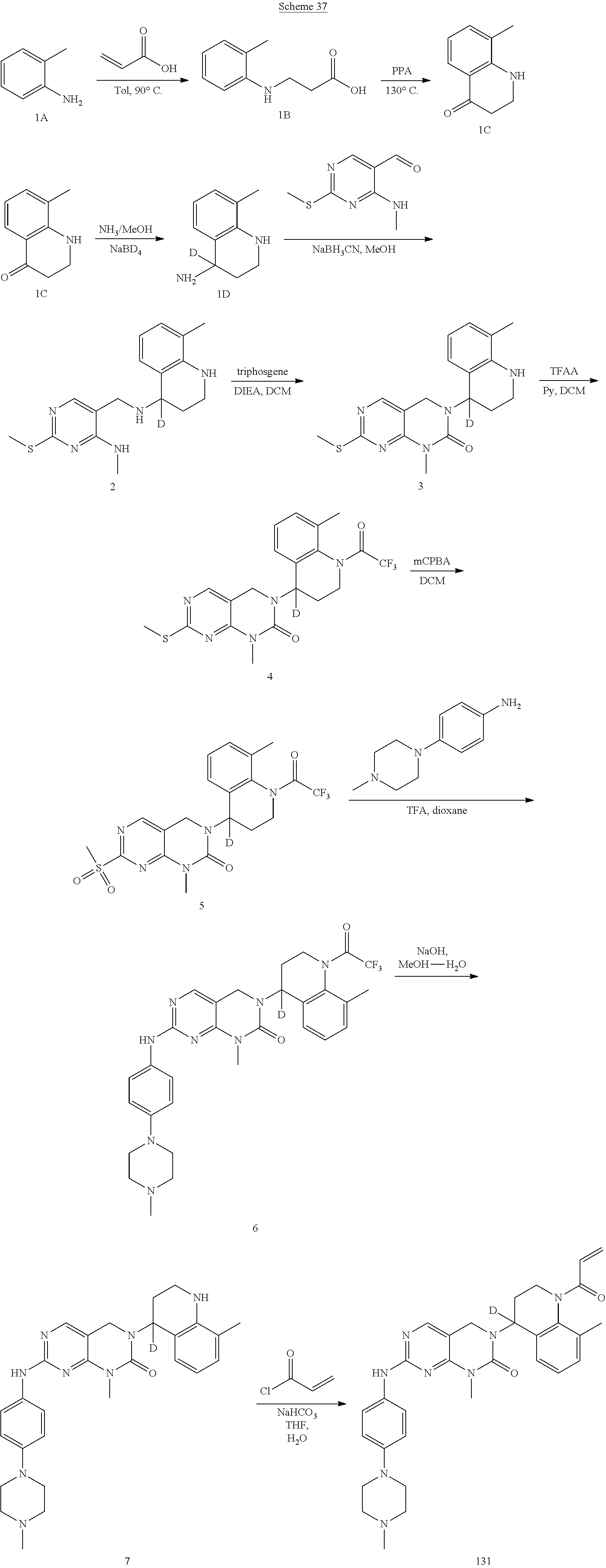

To a solution of 2-methylaniline (20 g, 93.3 mmol, 10.0 mL, 1 eq) in Tol. (200 mL) was added acrylic acid (12.1 g, 84.0 mmol, 5.8 mL, 0.9 eq). The mixture was stirred at 90° C. for 12 hr. LC-MS showed desired compound was detected. The reaction mixture was concentrated under reduced pressure to remove solvent. The residue was purified by flash silica gel chromatography (120 g Silica Flash Column, Eluent of 0-15% Ethyl acetate/Petroleum ether gradient @ 200 mL/min) to give 3-(2-methylanilino)propanoic acid (27 g, 150.7 mmol, 80.7% yield) as a yellow solid.

A mixture of 3-(2-methylanilino)propanoic acid (17 g, 94.86 mmol, 1 eq) in PPA (170 mL) was stirred at 110° C. for 8 hr. LC-MS showed desired compound was detected. The reaction mixture was quenched by addition NaOH (2 M) adjust to pH=7, and then diluted with H₂O (800 mL) and extracted with ethyl acetate (500 mL×3). The combined organic layers were washed with brine 400 mL, dried over Na₂SO₄, filtered and concentrated under reduced pressure to give a residue. The residue was purified by flash silica gel chromatography (120 g Silica Flash Column, Eluent of 0-15% Ethyl acetate/Petroleum ether gradient @ 120 mL/min) to give 8-methyl-2,3-dihydro-1H-quinolin-4-one (13.6 g, 84.4 mmol, 88.9% yield) as a brown solid.

To a solution of 8-methyl-2,3-dihydro-1 H-quinolin-4-one (3 g, 18.6 mmol, 1 eq) in MeOH (50 mL) was added Ti(i-PrO)₄ (15.8 g, 55.8 mmol, 16.5 mL, 3 eq) and NH₃/MeOH (10 M, 37.2 mL, 20 eq). The mixture was stirred at 20° C. for 12 hr. Then the mixture was added NaBD₄ (2.1 g, 56.2 mmol, 3 eq) at 0° C. The mixture was stirred at 20° C. for 3 hr. LC-MS showed desired compound was detected. The reaction mixture was concentrated under reduced pressure to remove solvent. The residue was diluted with H₂O 300 mL and extracted with ethyl acetate (300 mL×3). The combined organic layers were washed with brine (250 mL×1), dried over Na₂SO₄, filtered and concentrated under reduced pressure to give a residue. The residue was purified by prep-HPLC (FA condition) column: 330 g Agela C18;mobile phase: [water(FA)-ACN];B %:10-40% 25 min;40% 10 min, then purified by prep-HPLC (HCl condition) column: Waters Xbridge BEH C18 250×50 mm×10 um;mobile phase: [water(HCl)-ACN];B %: 1%-30%, 10 min to give 4-deuterio-8-methyl-2,3-dihydro-1 H-quinolin-4-amine (2.1 g, 12.9 mmol, 68.7% yield) as a brown oil.

To a solution of 4-deuterio-8-methyl-2,3-dihydro-1 H-quinolin-4-amine (1.0 g, 6.3 mmol, 1 eq) in MeOH (20 mL) was added Ti(Oi-Pr)₄ (5.4 g, 18.9 mmol, 5.6 mL, 3 eq), CH₃COOH (1.9 g, 31.6 mmol, 1.8 mL, 5 eq) and 4-(methylamino)-2-methylsulfanyl-pyrimidine-5-carbaldehyde (1.2 g, 6.3 mmol, 1 eq). The mixture was stirred at 50° C. for 48 hr. Then NaBH₃CN (3.2 g, 50.5 mmol, 8 eq) was added to the mixture. The mixture was stirred at 20° C. for 3 hr. LC-MS showed desired compound was detected. The reaction mixture was concentrated under reduced pressure to remove solvent. The residue was diluted with NaHCO₃ 300 mL and extracted with ethyl acetate (300 mL×3). The combined organic layers were washed with brine (250 mL×1), dried over Na₂SO₄, filtered and concentrated under reduced pressure to give a residue. The residue was purified by flash silica gel chromatography (20 g Silica Flash Column, Eluent of 0-33% Ethyl acetate/Petroleum ether gradient @ 120 mL/min) to give 4-deuterio-8-methyl-N-[[4-(methylamino)-2-methylsulfanyl-pyrimidin-5-yl]methyl]-2,3-dihydro-1 H-quinolin-4-amine (1.1 g, 3.3 mmol, 52.8% yield) as a yellow solid.

To a solution of 4-deuterio-8-methyl-N-[[4-(methylamino)-2-methylsulfanyl-pyrimidin-5-yl]methyl]-2,3-dihydro-1H-quinolin-4-amine (1.1 g, 3.3 mmol, 1 eq) in DCM (15 mL) was added DIEA (2.2 g, 16.6 mmol, 2.9 mL, 5 eq) and triphosgene (987.7 mg, 3.3 mmol, 1 eq) at 0° C. The mixture was stirred at 20° C. for 12 hr. LC-MS showed desired compound was detected. The residue was diluted with NaHCO₃ 50 mL and extracted with dichloromethane (50 mL×3). The combined organic layers were washed with brine (300 mL×1), dried over Na₂SO₄, filtered and concentrated under reduced pressure to give a residue. The residue was purified by flash silica gel chromatography (20 g Silica Flash Column, Eluent of 0-25% Ethyl acetate/Petroleum ether gradient @ 100 mL/min) to give 3-(4-deuterio-8-methyl-2,3-dihydro-1H-quinolin-4-yl)-1-methyl-7-methylsulfanyl-4H-pyrimido[4,5-d]pyrimidin-2-one (900 mg, 2.5 mmol, 75.8% yield) as a white solid.

To a solution of 3-(4-deuterio-8-methyl-2,3-dihydro-1 H-quinolin-4-yl)-1-methyl-7-methylsulfanyl-4H-pyrimido[4,5-d]pyrimidin-2-one (400 mg, 1.1 mmol, 1 eq) in DCM (5 mL) was added Py (355.0 mg, 4.5 mmol, 362.3 uL, 4 eq) and TFAA (353.5 mg, 1.7 mmol, 234.1 uL, 1.5 eq) at 0° C. The mixture was stirred at 25° C. for 12 hr. LC-MS showed desired compound was detected. The residue was diluted with H₂O 50 mL and extracted with dichloromethane (40 mL×3). The combined organic layers were washed with brine (30 mL×1), dried over Na₂SO₄, filtered and concentrated under reduced pressure to give a residue.

The residue was purified by flash silica gel chromatography (12 g Silica Flash Column, Eluent of 0-11% Ethyl acetate/Petroleum ether gradient @ 100 mL/min) to give 3-[4-deuterio-8-methyl-1-(2,2,2-trifluoroacetyl)-2,3-dihydroquinolin-4-yl]-1-methyl-7-methylsulfanyl-4H-pyrimido[4,5-d]pyrimidin-2-one (500 mg, 1.1 mmol, 98.5% yield) as a yellow solid.

To a solution of 3-[4-deuterio-8-methyl-1-(2,2,2-trifluoroacetyl)-2,3-dihydroquinolin-4-yl]-1-methyl-7-methylsulfanyl-4H-pyrimido[4,5-d]pyrimidin-2-one (600 mg, 1.3 mmol, 1 eq) in DCM (10 mL) was added mCPBA (403.8 mg, 2.0 mmol, 85% purity, 1.5 eq) at 0° C. The mixture was stirred at 25° C. for 12 hr. LC-MS showed desired compound was detected. The reaction mixture was partitioned between Na₂SO₃ 30 mL and dichloromethane (30 mL×3).

The organic phase was separated, washed with NaHCO₃ (20 mL×1), dried over Na₂SO₄, filtered and concentrated under reduced pressure to give 3-[4-deuterio-8-methyl-1-(2,2,2-trifluoroacetyl)-2,3-dihydroquinolin-4-yl]-1-methyl-7-methylsulfonyl-4H-pyrimido[4,5-d]pyrimidin-2-one (530 mg, crude) as a white solid.

To a solution of 3-[4-deuterio-8-methyl-1-(2,2,2-trifluoroacetyl)-2,3-dihydroquinolin-4-yl]-1-methyl-7-methylsulfonyl-4H-pyrimido[4,5-d]pyrimidin-2-one (100 mg, 206.4 umol, 1 eq) in dioxane (2 mL) was added TFA (35.3 mg, 309.6 umol, 22.9 uL, 1.5 eq) and 4-(4-methylpiperazin-1-yl)aniline (47.4 mg, 247.7 umol, 1.2 eq). The mixture was stirred at 120° C. for 12 hr. LC-MS showed desired compound was detected. The reaction mixture was concentrated under reduced pressure to remove solvent. The residue was purified by prep-HPLC (TFA condition) column: Phenomenex C18 100×30 mm×5 um; mobile phase: [water(TFA)-ACN]; B %: 15%-45%, 8 min to give 3-[4-deuterio-8-methyl-1-(2,2,2-trifluoroacetyl)-2,3-dihydroquinolin-4-yl]-1-methyl-7-[4-(4-methylpiperazin-1-yl)anilino]-4H-pyrimido[4,5-d]pyrimidin-2-one (250 mg, 419.7 umol, 40.7% yield) as a brown solid.

To a solution of 3-[4-deuterio-8-methyl-1-(2,2,2-trifluoroacetyl)-2,3-dihydroquinolin-4-yl]-1-methyl-7-[4-(4-methylpiperazin-1-yl)anilino]-4H-pyrimido[4,5-d]pyrimidin-2-one (200 mg, 335.8 umol, 1 eq) in MeOH (3 mL)/H₂O (1 mL) was added NaOH (67.2 mg, 1.7 mmol, 5 eq). The mixture was stirred at 50° C. for 8 hr. LC-MS showed desired compound was detected. The reaction mixture was diluted with H₂O 10 mL and extracted with chloroform: Isopropyl alcohol=3:1 (10 mL×5). The combined organic layers were washed with brine (10 mL×1), dried over Na₂SO₄, filtered and concentrated under reduced pressure to give 3-(4-deuterio-8-methyl-2,3-dihydro-1 H-quinolin-4-yl)-1-methyl-7-[4-(4-methylpiperazin-1-yl)anilino]-4H-pyrimido[4,5-d]pyrimidin-2-one (120 mg, crude) as a purple solid.

To a solution of 3-(4-deuterio-8-methyl-2,3-dihydro-1 H-quinolin-4-yl)-1-methyl-7-[4-(4-methylpiperazin-1-yl)anilino]-4H-pyrimido[4,5-d]pyrimidin-2-one (60 mg, 120.1 umol, 1 eq) and NaHCO₃ (30.3 mg, 360.3 umol, 14.0 uL, 3 eq) in THE (1 mL)/H₂O (1 mL) was added prop-2-enoyl chloride (10.9 mg, 120.1 umol, 9.8 uL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 1%-40%, 8 min, then purified by prep-HPLC (neutral condition) column: Waters Xbridge BEH C18 100×30 mm×10 um; mobile phase: [water(NH₄HCO₃)-ACN]; B %: 15%-55%, 8 min to give 3-(8-hydroxy-1-prop-2-enoyl-3,4-dihydro-2H-quinolin-4-yl)-1-methyl-7-[4-(4-methylpiperazin-1-yl)anilino]-4H-pyrimido[4,5-d]pyrimidin-2-one (17.2 mg, 31.0 umol, 17.2% yield, 100% purity) as a yellow solid.

### Procedure for Preparation of Compound 134

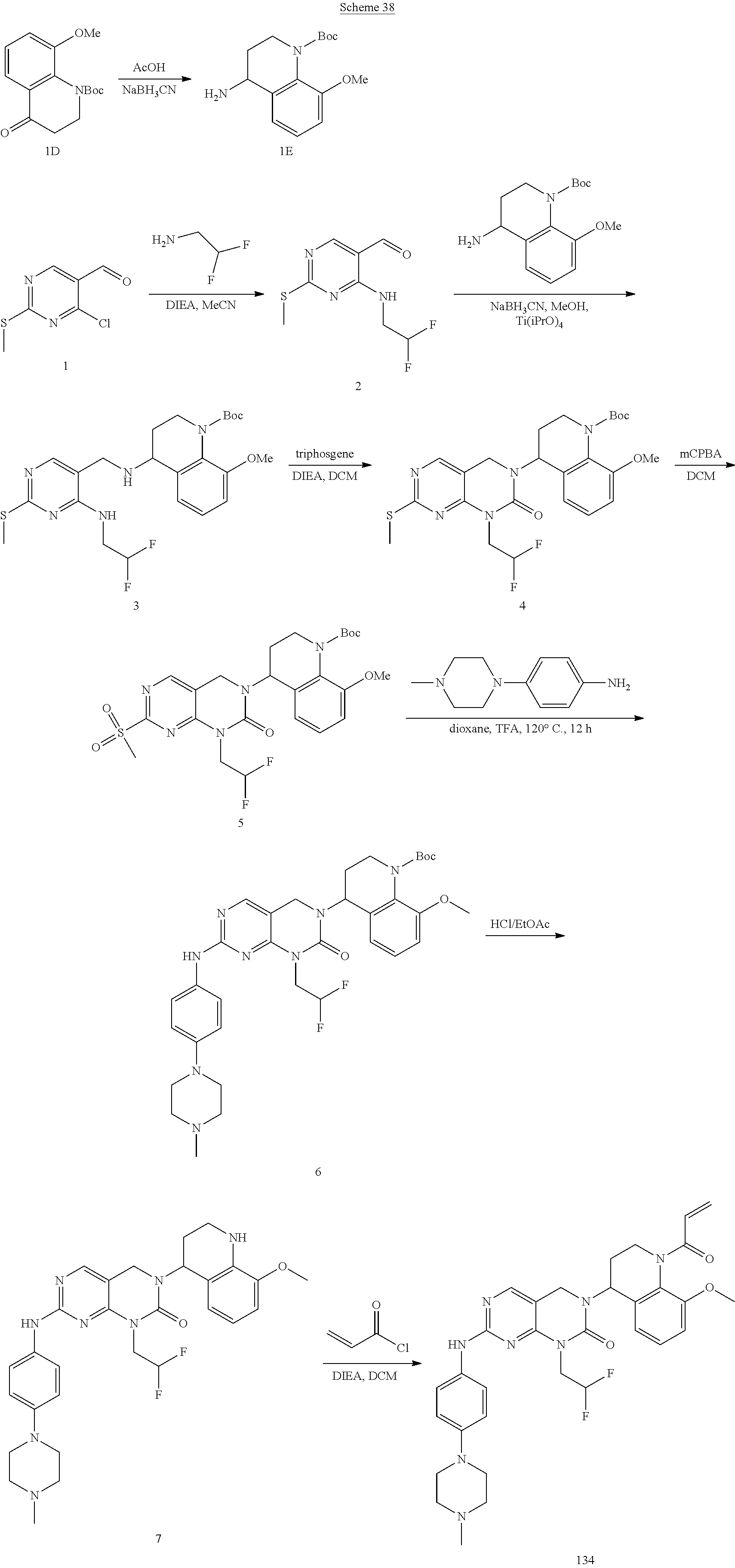

To a solution of tert-butyl 8-methoxy-4-oxo-2,3-dihydroquinoline-1-carboxylate(500 mg, 1.8 mmol, 1 eq) in MeOH(30 mL) was added NH40Ac (2.8 g, 36.1 mmol, 20 eq), AcOH(216.6 mg, 3.6 mmol, 206.2 uL, 2 eq) and NaBH₃CN (566.5 mg, 9.0 mmol, 5 eq). The mixture was stirred at 75° C. for 12 hr. LC-MS showed desired mass was detected. The reaction mixture was concentrated under reduced pressure to remove solvent. The residue was diluted with NaHCO₃ (200 mL) and extracted with ethyl acetate (200 mL×3). The combined organic layers were washed with aqueous NaCl (100 mL×1), dried over Na₂SO₄, filtered and concentrated under reduced pressure to give a residue. The residue was purified by flash silica gel chromatography (20 g Silica Flash Column, Eluent of 0-100% Ethyl acetate/Petroleum ether gradient @ 100 mL/min) to yield compound tert-butyl 4-amino-8-methoxy-3,4-dihydro-2H-quinoline-1-carboxylate(1 g, 3.6 mmol, 99.6% yield) obtained as a white solid.

To a solution of 4-chloro-2-methylsulfanyl-pyrimidine-5-carbaldehyde(4.7 g, 24.7 mmol, 1 eq) in MeCN(50 mL) was added DIEA(9.6 g, 74.0 mmol, 12.9 mL, 3 eq) and 2,2-difluoroethanamine(2 g, 24.7 mmol, 1 eq). The mixture was stirred at 50° C. for 12 hr. The reaction was monitored by LCMS which showed the reactant was consumed completely and the desired mass peak was detected. The reaction mixture was concentrated under reduced pressure to remove solvent. The residue was purified by flash silica gel chromatography(40 g SepaFlash® Silica Flash Column, Eluent of 0-100% Ethyl acetate/Petroleum ether gradient @ 120 mL/min) to give Compound 4-(2,2-difluoroethylamino)-2-methylsulfanyl-pyrimidine-5-carbaldehyde(4.8 g, 20.6 mmol, 83.4% yield) obtained as a white solid.

To a solution of 4-(2,2-difluoroethylamino)-2-methylsulfanyl-pyrimidine-5-carbaldehyde(837.9 mg, 3.6 mmol, 1 eq) in MeOH (2 mL) was added NaBH₃CN (2.3 g, 35.9 mmol, 10 eq), tert-butyl 4-amino-8-methoxy-3,4-dihydro-2H-quinoline-1-carboxylate(1 g, 3.6 mmol, 1 eq) and Ti(i-PrO)₄(3.1 g, 10.8 mmol, 3.2 mL, 3 eq). The mixture was stirred at 70° C. for 16 hr. The reaction was monitored by LCMS which showed complete consumption of reactant and detection of the desired mass peak. The reaction mixture was concentrated under reduced pressure to remove solvent. The residue was diluted with NaHCO₃ 50 mL and extracted with ethyl acetate (50 mL×3). The combined organic layers were washed with NaCl(40 mL×2), dried over Na₂SO₄, filtered and concentrated under reduced pressure to give a residue. The residue was purified by flash silica gel chromatography(12 g Silica Flash Column, Eluent of 0-100% Ethyl acetate/Petroleum ether gradient @ 80 mL/min) to give compound tert-butyl 4-[[4-(2,2-difluoroethylamino)-2-methylsulfanyl-pyrimidin-5-yl]methylamino]-8-methoxy-3,4-dihydro-2H-quinoline-1-carboxylate (1.2 g, 2.4 mmol, 67.4% yield) obtained as a yellow solid.

To a solution of tert-butyl 4-[[4-(2,2-difluoroethylamino)-2-methylsulfanyl-pyrimidin-5-yl]methylamino]-8-methoxy-3,4-dihydro-2H-quinoline-1-carboxylate (1.5 g, 3.0 mmol, 1 eq) in DCM (12 mL) was added DIEA (1.2 g, 9.1 mmol, 1.6 mL, 3 eq) and triphosgene (898.2 mg, 3.0 mmol, 1 eq) at 0° C. The mixture was stirred at 25° C. for 12 hr. The reaction was monitored by LCMS which showed complete consumption of reactant and detection of the desired mass peak. The residue was diluted with NaHCO₃ (30 mL) and extracted with dichloromethane (40 mL×3). The combined organic layers were washed with brine(40 mL×1), dried over Na₂SO₄, filtered and concentrated under reduced pressure to give Compound tert-butyl 4-[1-(2,2-difluoroethyl)-7-methylsulfanyl-2-oxo-4H-pyrimido[4,5-d]pyrimidin-S-yl]-8-methoxy-3,4-dihydro-2H-quinoline-1-carboxylate (1.7 g, crude) obtained as a white solid.

To a solution of tert-butyl 4-[1-(2,2-difluoroethyl)-7-methylsulfanyl-2-oxo-4H-pyrimido[4,5-d]pyrimidin-S-yl]-8-methoxy-3,4-dihydro-2H-quinoline-1-carboxylate(1.8 g, 3.5 mmol, 1 eq) in DCM(18 mL) was added m-CPBA(910.8 mg, 4.5 mmol, 85% purity, 1.3 eq) at 0° C. The mixture was stirred at 25° C. for 2 hr. The reaction was monitored by LCMS which showed complete consumption of reactant and detection of the desired product. The reaction mixture was concentrated under reduced pressure to remove solvent. The residue was diluted with Na₂SO₃ (20 mL) and extracted with dichloromethane (20 mL×3). The combined organic layers were washed with NaHCO₃ (20 mL×2), dried over Na₂SO₄, filtered and concentrated under reduced pressure to give tert-butyl 4-[1-(2,2-difluoroethyl)-7-methylsulfonyl-2-oxo-4H-pyrimido[4,5-d]pyrimidin-S-yl]-8-methoxy-3,4-dihydro-2H-quinoline-1-carboxylate(1.7 g, crude) obtained as a white solid.

To a solution of tert-butyl 4-[1-(2,2-difluoroethyl)-7-methylsulfonyl-2-oxo-4H-pyrimido[4,5-d]pyrimidin-S-yl]-8-methoxy-3,4-dihydro-2H-quinoline-1-carboxylate(200.0 mg, 361.3 umol, 1 eq) in dioxane (3 mL) was added TFA(41.2 mg, 361.3 umol, 26.8 uL, 1 eq) and 4-(4-methylpiperazin-1-yl)aniline(82.9 mg, 433.5 umol, 1.2 eq). The mixture was stirred at 120° C. for 12 hr(4×batch). The reaction was monitored by LCMS which showed complete consumption of reactant and detection of the desired product. The reaction mixture was concentrated under reduced pressure to remove solvent. The residue was purified by prep-HPLC (TFA condition; column: Phenomenex Luna C18 100×30 mm×5 um; mobile phase: [water(TFA)-ACN]; B %: 25%-55%, 8 min) to compound tert-butyl 4-[1-(2,2-difluoroethyl)-7-[4-(4-methylpiperazin-1-yl)anilino]-2-oxo-4H-pyrimido[4,5-d]pyrimidin-S-yl]-8-methoxy-3,4-dihydro-2H-quinoline-1-carboxylate(180 mg, 270.8 umol, 25.0% yield) obtained as a brown solid.

A mixture of tert-butyl 4-[1-(2,2-difluoroethyl)-7-[4-(4-methylpiperazin-1-yl)anilino]-2-oxo-4H-pyrimido[4,5-d]pyrimidin-S-yl]-8-methoxy-3,4-dihydro-2H-quinoline-1-carboxylate(173.7 mg, 261.3 umol, 1 eq) in HCl/EtOAc(3 mL, 4M) was stirred at 25° C. for 2 hr. The reaction was monitored by LCMS which showed complete consumption of reactant and detection of the desired product. The reaction mixture was concentrated under reduced pressure to give Compound 1-(2,2-difluoroethyl)-S-(8-methoxy-1,2,3,4-tetrahydroquinolin-4-yl)-7-[4-(4-methylpiperazin-1-yl)anilino]-4H-pyrimido[4,5-d]pyrimidin-2-one (170 mg, crude) obtained as a brown solid.

To a solution of 1-(2,2-difluoroethyl)-S-(8-methoxy-1,2,3,4-tetrahydroquinolin-4-yl)-7-[4-(4-methylpiperazin-1-yl)anilino]-4H-pyrimido[4,5-d]pyrimidin-2-one(60 mg, 106.3 umol, 1 eq) in DCM(1 mL) was added DIEA(41.2 mg, 318.8 umol, 55.5 uL, 3 eq) and prop-2-enoyl chloride(9.6 mg, 106.3 umol, 8.7 uL, 1 eq) at 0° C. The mixture was stirred at 25° C. for 1 hr. The reaction was monitored by LCMS which showed complete consumption of reactant and detection of the desired mass peak. The residue was purified by prep-HPLC (TFA condition; column: Phenomenex Luna C18 100×30 mm×5 um; mobile phase: [water(TFA)-ACN];B %: 10%-35%, 8 min) to yield 3-(1-acryloyl-8-methoxy-1,2,3,4-tetrahydroquinolin-4-yl)-1-(2,2-difluoroethyl)-7-((4-(4-methylpiperazin-1-yl)phenyl)amino)-3,4-dihydropyrimido[4,5-d]pyrimidin-2(1H)-one(12 mg, 19.1 umol, 18.0% yield, 98.4% purity) as a white solid.

### Procedure for Preparation of Compound 139

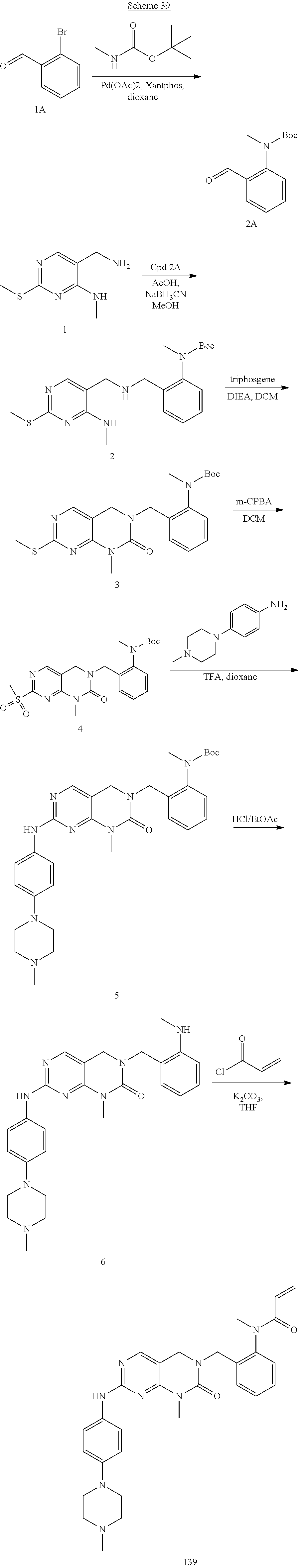

A mixture of 2-bromobenzaldehyde (2 g, 10.8 mmol, 1.2 mL, 1 eq), tert-butyl N-methylcarbamate (1.6 g, 11.9 mmol, 1.1 eq), Cs₂CO₃ (5.3 g, 16.2 mmol, 1.5 eq), Pd(OAc)₂ (169.9 mg, 756.7 umol, 0.07 eq) and Xantphos (625.5 mg, 1.1 mmol, 0.1 eq) in dioxane (100 mL) was degassed and purged with N₂ for 3 times at 20° C., and then the mixture was stirred at 85° C. for 12 hr under N₂ atmosphere. TLC indicated reactants was consumed completely and four new spots formed (Petroleum ether/Ethyl acetate=3/1, R_{f}^{P}=0.50). The reaction mixture was concentrated under reduced pressure to remove solvent. The residue was diluted with H₂O (200 mL) and extracted with ethyl acetate (200 mL×3). The combined organic layers were washed with aqueous NaCl (100 mL×1), dried over Na₂SO₄, filtered and concentrated under reduced pressure to give a residue. The residue was purified by flash silica gel chromatography (20 g Silica Flash Column, Eluent of 0-100% Ethyl acetate/Petroleum ether gradient @ 100 mL/min) to yield compound tert-butyl N-(2-formylphenyl)-N-methyl-carbamate (6 g, 39.5 mmol, 73.1% yield, 93% purity) obtained as a yellow oil.

To a solution of 5-(aminomethyl)-N-methyl-2-methylsulfanyl-pyrimidin-4-amine (587.4 mg, 3.2 mmol, 1 eq) in MeOH (9 mL) was added AcOH (1.2 g, 19.1 mmol, 1.1 mL, 6 eq) , tert-butyl N-(2-formylphenyl)-N-methyl-carbamate (100 mg, 425.0 umol, 1 eq),Ti(i-PrO)₄ (2.7 g, 9.6 mmol, 2.8 mL, 3 eq), tert-butyl N-(2-formylphenyl)-N-methyl-carbamate (750 mg, 3.2 mmol, 1 eq) and NaBH₃CN (601.0 mg, 9.6 mmol, 3 eq). The mixture was stirred at 25° C. for 12 hr. The reaction was monitored by LCMS which showed the reactant was consumed completely and the desired mass peak was detected. The reaction mixture was concentrated under reduced pressure to remove solvent. The residue was diluted with NH₄Cl (20 mL) and extracted with ethyl acetate (20 mL×3). The combined organic layers were washed with NaCl (20 mL×3), dried over Na₂SO₄, filtered and concentrated under reduced pressure to give a residue. The residue was purified by flash silica gel chromatography (12 g Silica Flash Column, Eluent of 0-100% THE/Petroleum ether gradient @ 80 mL/min) to give compound tert-butyl N-methyl-N-[2-[[[4-(methylamino)-2-methylsulfanyl-pyrimidin-5-yl]methylamino]methyl]phenyl]carbamate (700 mg, 1.2 mmol, 19.4% yield) obtained as a white solid.

To a solution of tert-butyl N-methyl-N-[2-[[[4-(methylamino)-2-methylsulfanyl-pyrimidin-5-yl]methylamino]methyl]phenyl]carbamate (700 mg, 1.7 mmol, 1 eq) in DCM (10 mL) was added DIEA (896.8 mg, 6.9 mmol, 1.2 mL, 4 eq) and triphosgene (411.8 mg, 1.4 mmol, 0.8 eq) at 0° C. The mixture was stirred at 25° C. for 1 hr. The reaction was monitored by LCMS which showed complete consumption of reactant and detection of the desired mass peak.

The residue was diluted with NaHCO₃ (30 mL) and extracted with dichloromethane (40 mL×3). The combined organic layers were washed with brine (40 mL×1), dried over Na₂SO₄, filtered and concentrated under reduced pressure to give a residue to give compound tert-butyl N-methyl-N-[2-[(1-methyl-7-methylsulfanyl-2-oxo-4H-pyrimido[4,5-d]pyrimidin-S-yl)methyl]phenyl]carbamate (670 mg, crude) obtained as a yellow solid.

To a solution of tert-butyl N-methyl-N-[2-[(1-methyl-7-methylsulfanyl-2-oxo-4H-pyrimido[4,5-d]pyrimidin-S-yl)methyl]phenyl]carbamate (660 mg, 1.5 mmol, 1 eq) in DCM (6 mL) was added m-CPBA (405.5 mg, 2.0 mmol, 85% purity, 1.3 eq) at 0° C. The mixture was stirred at 25° C. for 12 hr. The reaction was monitored by LCMS which showed complete consumption of reactant and detection of the desired mass peak. The residue was diluted with Na₂SO₃ (20 mL) and extracted with dichloromethane (20 mL×3). The combined organic layers were washed with NaHCO₃ (20 mL×2), dried over Na₂SO₄, filtered and concentrated under reduced pressure to give a residue. The residue was purified by column chromatography (SiO2, Petroleum ether/Ethyl acetate=3/1) to give compound tert-butyl N-methyl-N-[2-[(1-methyl-7-methylsulfinyl-2-oxo-4H-pyrimido[4,5-d]pyrimidin-S-yl)methyl]phenyl]carbamate (200 mg, 448.9 umol, 29.2% yield) obtained as a yellow solid.

To a solution of tert-butyl N-methyl-N-[2-[(1-methyl-7-methylsulfonyl-2-oxo-4H-pyrimido[4,5-d]pyrimidin-S-yl)methyl]phenyl]carbamate (200 mg, 433.3 umol, 1 eq) in dioxane (2 mL) was added TFA (59.3 mg, 520.0 umol, 38.5 uL, 1.2 eq) and 4-(4-methylpiperazin-1-yl)aniline (99.5 mg, 520.0 umol, 1.2 eq). The mixture was stirred at 120° C. for 12 hr. The reaction was monitored by LCMS which showed complete consumption of reactant and detection of the desired product. The reaction mixture was concentrated under reduced pressure to remove solvent. The residue was diluted with DMF (2 mL). The residue was purified by prep-HPLC (FA condition; column: C18(75×30 mm×3 um); mobile phase: [water(FA)-ACN];B %: 5%-35%, 8 min) to give tert-butyl N-methyl-N-[2-[[1-methyl-7-[4-(4-methylpiperazin-1-yl) anilino]-2-oxo-4H-pyrimido[4,5-d]pyrimidin-S-yl]methyl]phenyl]carbamate (80 mg, 139.7 umol, 62.2% yield) as a white solid.

A solution of tert-butyl N-methyl-N-[2-[[1-methyl-7-[4-(4-methylpiperazin-1-yl)anilino]-2-oxo-4H-pyrimido[4,5-d]pyrimidin-S-yl]methyl]phenyl]carbamate (40 mg, 69.8 umol, 1 eq) in HCl/EtOAc (2 mL, 4 M) was stirred at 25° C. for 10 min. The reaction was monitored by LCMS which showed complete consumption of reactant and detection of the desired product. The reaction mixture was concentrated under reduced pressure to give 1-methyl-S-[[2-(methylamino)phenyl]methyl]-7-[4-(4-methylpiperazin-1-yl)anilino]-4H-pyrimido[4,5-d]pyrimidin-2-one (50 mg, crude) as a yellow solid used into the next step without further purification.

To a solution of 1-methyl-S-[[2-(methylamino)phenyl]methyl]-7-[4-(4-methylpiperazin-1-yl)anilino]-4H-pyrimido[4,5-d]pyrimidin-2-one (50 mg, 105.8 umol, 1 eq) in THE (1.5 mL) was added K₂CO₃ (73.1 mg, 529.0 umol, 5 eq) and prop-2-enoyl chloride (9.6 mg, 105.8 umol, 8.6 uL, 1 eq) at 0° C. The mixture was stirred at 50° C. for 12 hr. The reaction was monitored by LCMS which showed complete consumption of reactant and detection of the desired mass peak. The reaction mixture was concentrated under reduced pressure to remove solvent. The residue was diluted with DMF (3 mL) and purified by prep-HPLC (neutral condition; column: Phenomenex C18(75×30 mm×3 um);mobile phase: [water(NH₄HCO₃)-ACN];B %: 5%-50%, 8 min) to yield compound N-methyl-N-[2-[[1-methyl-7-[4-(4-methylpiperazin-1-yl)anilino]-2-oxo-4H-pyrimido[4,5-d]pyrimidin-S-yl]methyl]phenyl]prop-2-enamide (10 mg, 17.9 umol, 16.9% yield, 94.2% purity) obtained as a white solid.

### Procedure for Preparation of Compound 135

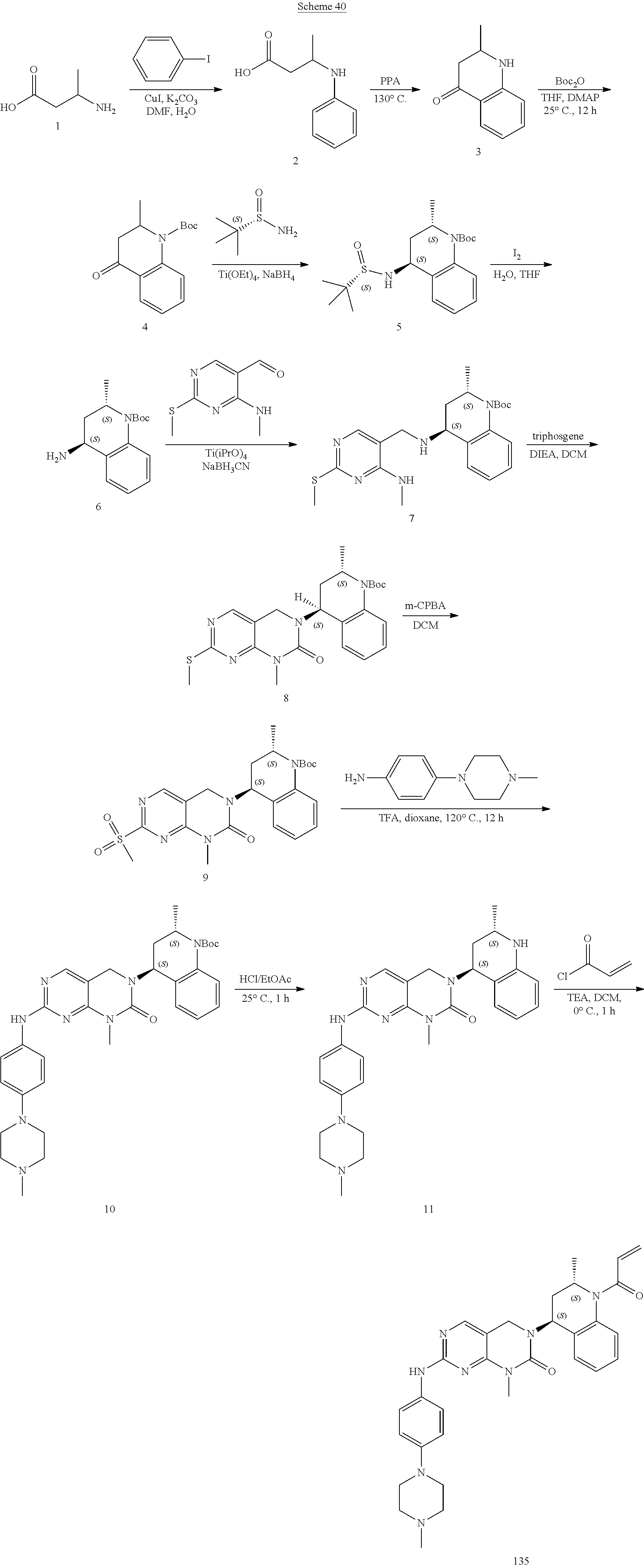

To a solution of iodobenzene (20 g, 24.5 mmol, 2.7 mL, 1 eq) and 3-aminobutanoic acid (10.1 g, 24.5 mmol, 1 eq) in DMF (200 mL) and H₂O (4 mL) was added CuI (1.9 g, 2.45 mmol, 0.1 eq) and K₂CO₃ (33.9 g, 61.3 mmol, 2.5 eq). The mixture was stirred at 100° C. for 24 hr under N₂. LC-MS showed the desired compound was detected. The reaction mixture was acidified to pH=4 with HCl (3 M). The reaction mixture was partitioned between H₂O (400 mL) and ethyl acetate (400 mL×3). The organic phase was separated, washed with brine (200 mL×1), dried over Na₂SO₄, filtered and concentrated under reduced pressure to give a residue. The residue was purified by flash silica gel chromatography (80 g Silica Flash Column, Eluent of 0-20% Ethyl acetate/Petroleum ether gradient @ 150 mL/min) to give 3-anilinobutanoic acid (12.5 g, 69.7 mmol, 71.1% yield) as a light yellow oil.

A solution of 3-anilinobutanoic acid (10 g, 27.90 mmol, 1 eq) in PPA (100 mL) was stirred at 130° C. for 2 hr. LC-MS showed the desired compound was detected. The reaction mixture was quenched by addition NaOH (2 M) adjust to pH=8, and then diluted with H₂O (500 mL) and extracted with ethyl acetate (1000 mL×3). The combined organic layers were washed with brine (800 mL×1), dried over Na₂SO₄, filtered and concentrated under reduced pressure to give a residue. The residue was purified by flash silica gel chromatography (80 g Silica Flash Column, Eluent of 0-10% Ethyl acetate/Petroleum ether gradient @ 120 mL/min) to give 2-methyl-2,3-dihydro-1 H-quinolin-4-one (7 g) as a yellow solid.

To a solution of 2-methyl-2,3-dihydro-1H-quinolin-4-one (3 g, 18.6 mmol, 1 eq) in THE (50 mL) was added (Boc)₂0 (6.1 g, 27.9 mmol, 6.4 mL, 1.5 eq), TEA (5.6 g, 55.8 mmol, 7.8 mL, 3 eq) and DMAP (227.3 mg, 1.9 mmol, 0.1 eq). The mixture was stirred at 25° C. for 12 hr. LC-MS showed the desired compound was detected. The reaction mixture was concentrated under reduced pressure to give a residue. The residue was purified by flash silica gel chromatography (20 g Silica Flash Column, Eluent of 0-20% Ethyl acetate/Petroleum ether gradient @ 100 mL/min) to give tert-butyl 2-methyl-4-oxo-2, 3-dihydroquinoline-1-carboxylate (1.2 g, 4.6 mmol, 24.6% yield) as a light yellow solid.

To a solution of tert-butyl 2-methyl-4-oxo-2, 3-dihydroquinoline-1-carboxylate (1.2 g, 4.6 mmol, 1 eq) in THE (20 mL) was added Ti(OEt)₄ (10.5 g, 45.9 mmol, 9.5 mL, 10 eq) and (S)-2-methylpropane-2-sulfinamide (1.7 g, 13.8 mmol, 3 eq). The mixture was stirred at 75° C. for 12 hr. The reaction was cooled down to 0° C., then NaBH₄ (1.0 g, 27.6 mmol, 6 eq) was added to the mixture at 0° C. The mixture was stirred for 3 hr and was allowed to warm to 25° C. LC-MS showed the desired compound was detected. The reaction mixture was quenched by addition of NH₄Cl solution (50 mL), and extracted with ethyl acetate (50 mL×3). The combined organic layers were washed with brine (30 mL×1), dried over Na₂SO₄, filtered and concentrated under reduced pressure to give tert-butyl (4S)-4-[[(S)-tert-butylsulfinyl]amino]-2-methyl-3,4-dihydro-2H-quinoline-1-carboxylate (1.2 g, 3.3 mmol, 71.3% yield) as a light yellow solid.

To a solution of tert-butyl (4S)-4-[[(S)-tert-butylsulfinyl]amino]-2-methyl-3,4-dihydro-2H-quinoline-1-carboxylate (1.2 g, 3.3 mmol, 1 eq) in THE (10 mL) and H₂O (2 mL) was added 12 (166.2 mg, 654.8 umol, 131.9 uL, 0.2 eq). The mixture was stirred at 50° C. for 12 hr. LC-MS showed the desired compound was detected. The reaction mixture was partitioned between Na₂SO₃ (50 mL) and ethyl acetate (50 mL×3). The organic phase was separated, dried over Na₂SO₄, filtered and concentrated under reduced pressure to give tert-butyl (2S, 4S)-4-amino-2-methyl-3,4-dihydro-2H-quinoline-1-carboxylate (1 g, crude) as a yellow oil.

To a solution of 4-(methylamino)-2-methylsulfanyl-pyrimidine-5-carbaldehyde (698.4 mg, 3.8 mmol, 1 eq) and tert-butyl (2S, 4S)-4-amino-2-methyl-3,4-dihydro-2H-quinoline-1-carboxylate (1 g, 3.8 mmol, 1 eq) in MeOH (10 mL) was added Ti(iPrO)₄ (3.3 g, 11.4 mmol, 3.4 mL, 3 eq) and AcOH (1.4 g, 22.9 mmol, 1.3 mL, 6 eq). The mixture was stirred at 65° C. for 12 hr. Then NaBH₃CN (1.20 g, 19.06 mmol, 5 eq) was added to the mixture, the mixture was stirred at 65° C. for 3 hr. LC-MS showed the desired compound was detected. The reaction mixture was partitioned between NaHCO₃ (100 mL) and ethyl acetate (100 mL×3). The organic phase was separated, washed with brine (50 mL×1), dried over Na₂SO₄, filtered and concentrated under reduced pressure to give a residue. The residue was purified by flash silica gel chromatography (20 g Silica Flash Column, Eluent of 0-30% Ethyl acetate/Petroleum ether gradient @ 120 mL/min) to give tert-butyl (2S, 4S)-2-methyl-4-[[4-(methylamino)-2-methylsulfanyl-pyrimidin-5-yl]methylamino]-3,4-dihydro-2H-quinoline-1-carboxylate (1.3 g, 3.0 mmol, 78.2% yield) as a light yellow oil.

To a solution of tert-butyl (2S, 4S)-2-methyl-4-[[4-(methylamino)-2-methylsulfanyl-pyrimidin-5-yl]methylamino]-3,4-dihydro-2H-quinoline-1-carboxylate (1.3 g, 3.0 mmol, 1 eq) in DCM (20 mL) was added DIEA (1.6 g, 11.9 mmol, 2.1 mL, 4 eq) and triphosgene (707.4 mg, 2.4 mmol, 0.8 eq) at 0° C. The mixture was stirred at 25° C. for 2 hr. LC-MS showed the desired compound was detected. The reaction mixture was partitioned between NaHCO₃ (100 mL) and dichloromethane (100 mL×2). The organic phase was separated, washed with brine (30 mL×1), dried over Na₂SO₄, filtered and concentrated under reduced pressure to give tert-butyl (2S, 4S)-2-methyl-4-(1-methyl-7-methylsulfanyl-2-oxo-4H-pyrimido[4,5-d]pyrimidin-S-yl)-3,4-dihydro-2H-quinoline-1-carboxylate (1.5 g, crude) as a yellow oil.

To a solution of tert-butyl (2S, 4S)-2-methyl-4-(1-methyl-7-methylsulfanyl-2-oxo-4H-pyrimido[4,5-d]pyrimidin-S-yl)-3,4-dihydro-2H-quinoline-1-carboxylate (1.5 g, 3.3 mmol, 1 eq) in DCM (30 mL) was added mCPBA (869.0 mg, 4.3 mmol, 85% purity, 1.3 eq) at 0° C. The mixture was stirred at 25° C. for 12 hr. LC-MS showed the desired compound was detected.

The reaction mixture was partitioned between Na₂SO₃ (100 mL) and dichloromethane (100×3 mL). The organic phase was separated, washed with NaHCO₃ (50 mL×3), dried over Na₂SO₄, filtered and concentrated under reduced pressure to give tert-butyl (2S, 4S)-2-methyl-4-(1-methyl-7-methylsulfonyl-2-oxo-4H-pyrimido[4,5-d]pyrimidin-S-yl)-3,4-dihydro-2H-quinoline-1-carboxylate (1.6 g, crude) as a light yellow oil.

To a solution of tert-butyl (2S, 4S)-2-methyl-4-(1-methyl-7-methylsulfonyl-2-oxo-4H-pyrimido[4,5-d]pyrimidin-S-yl)-3,4-dihydro-2H-quinoline-1-carboxylate (800 mg, 410.2 umol, 1 eq) in dioxane (8 mL) was added 4-(4-methylpiperazin-1-yl)aniline (576.8 mg, 492.2 umol, 1.2 eq) and TFA (280.7 mg, 615.3 umol, 45.5 uL, 1.5 eq). The mixture was stirred at 120° C. for 12 hr. LC-MS showed the desired compound was detected. The residue was purified by prep-HPLC (TFA condition, column: Phenomenex Luna C18 100×30 mm×5 um; mobile phase: [water(TFA)-ACN];B %: 20%-55%, 8 min) to give tert-butyl (2S, 4S)-2-methyl-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (400 mg, 668.0 umol, 40.72% yield) as a brown solid.

A mixture of tert-butyl (2S, 4S)-2-methyl-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (400 mg, 668.0 umol, 1 eq) in HCl/EtOAc (4 mL, 4 M) was stirred at 25° C. for 1 hr. LC-MS showed the desired compound was detected. The reaction mixture was concentrated under reduced pressure to give 1-methyl-7-[4-(4-methylpiperazin-1-yl)anilino]-S-[(2S, 4S)-2-methyl-1,2,3,4-tetrahydroquinolin-4-yl]-4H-pyrimido[4,5-d]pyrimidin-2-one (380 mg, crude, HCl) as a light brown solid.

To a solution of 1-methyl-7-[4-(4-methylpiperazin-1-yl)anilino]-S-[(2S, 4S)-2-methyl-1,2,3,4-tetrahydroquinolin-4-yl]-4H-pyrimido[4,5-d]pyrimidin-2-one (150 mg, 280.3 umol, 1 eq, HCl) in DCM (4 mL) was added TEA (113.4 mg, 1.1 mmol, 156.1 uL, 4 eq) and prop-2-enoyl chloride (30.5 mg, 336.4 umol, 27.4 uL, 1.2 eq) at 0° C. The mixture was stirred at 0° C. for 1 hr. LC-MS showed the desired compound was detected. The reaction mixture was concentrated under reduced pressure to give a residue. The residue was purified by prep-HPLC (FA condition, column: Phenomenex Luna C18 100×30 mm×5 um; mobile phase: [water(FA)-ACN];B %: 10%-40%, 8 min) to give 1-methyl-7-[4-(4-methylpiperazin-1-yl)anilino]-3-[(2S,4S)-2-methyl-1-prop-2-enoyl-3,4-dihydro-2H-quinolin-4-yl]-4H-pyrimido[4,5-d]pyrimidin-2-one (69.3 mg, 122.4 umol, 43.7% yield, 97.6% purity) as a white solid.

### Procedure for Preparation of Compound 149

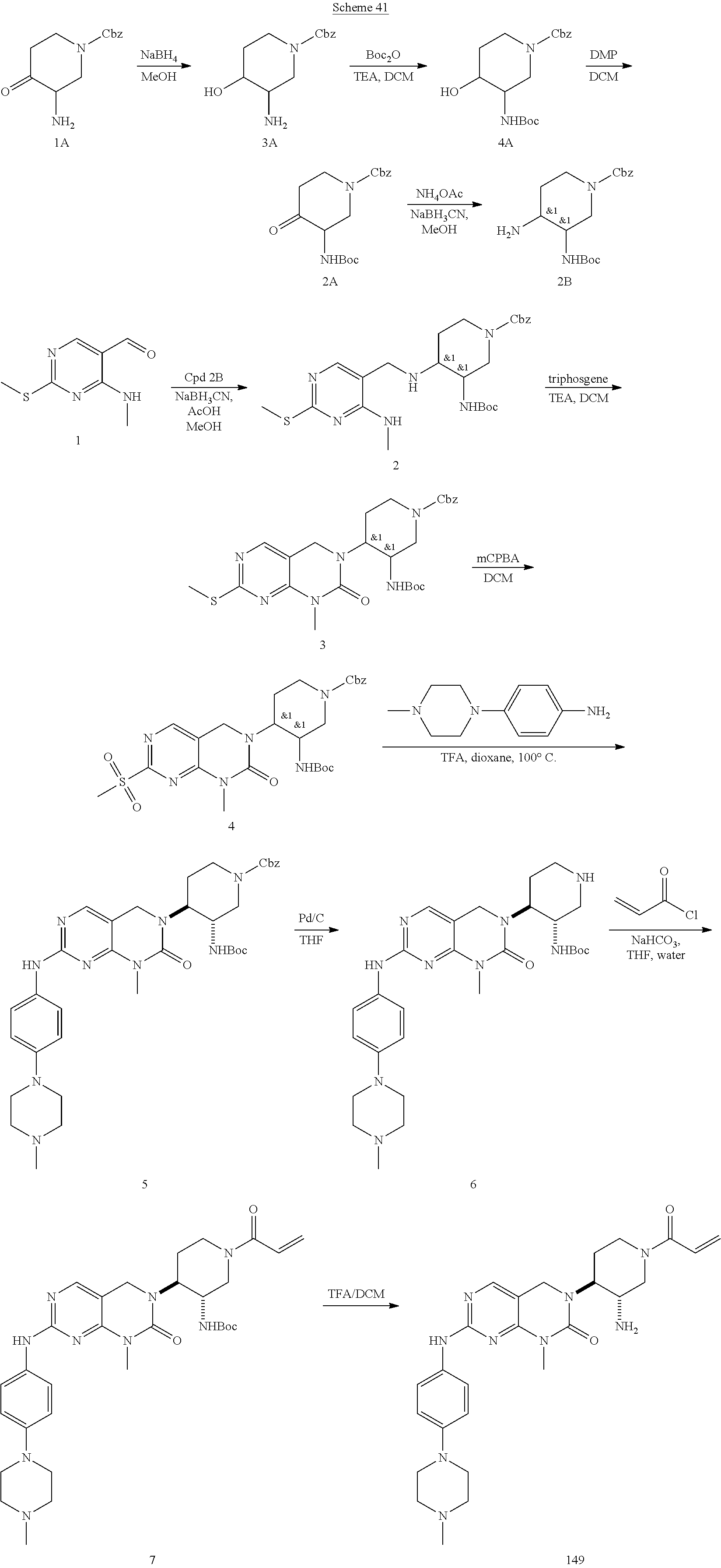

To a solution of benzyl 3-amino-4-oxo-piperidine-1-carboxylate (5 g, 17.6 mmol, 1 eq, HCl) in MeOH (50 mL) was added NaBH₄ (3.3 g, 87.8 mmol, 5 eq) at 0° C. The mixture was stirred at 0° C. for 3 hr. LC-MS showed desired compound was detected. The reaction mixture was concentrated under reduced pressure to remove solvent to give compound benzyl 3-amino-4-hydroxy-piperidine-1-carboxylate (5 g, crude) was obtained as a colorless oil.

To a solution of benzyl 3-amino-4-hydroxy-piperidine-1-carboxylate (5 g, 20.0 mmol, 1 eq) in DCM (20 mL) was added Boc₂O (5.23 g, 24.0 mmol, 5.5 mL, 1.2 eq) and TEA (5.05 g, 50.0 mmol, 7.0 mL, 2.5 eq). The mixture was stirred at 25° C. for 2 hr. LC-MS showed desired compound was detected. The mixture was concentrated to get a residue. The residue was purified by column chromatography (SiO₂, Petroleum ether/Ethyl acetate=10/1 to 1/1) to give compound benzyl 3-(tert-butoxycarbonylamino)-4-hydroxy-piperidine-1-carboxylate (5.5 g, 15.70 mmol, 78.57% yield) was obtained as a white solid.

To a solution of benzyl 3-(tert-butoxycarbonylamino)-4-hydroxy-piperidine-1-carboxylate (2.7 g, 7.7 mmol, 1 eq) in DCM (100 mL) was added Dess-Martin (4.3 g, 10.0 mmol, 1.3 eq). The mixture was stirred at 25° C. for 12 hr. LC-MS showed desired compound was detected. The mixture was quenched by Sat. NaSO₃ 50 ml and then extracted by DCM (300 mL×3), the organic layers was washed by Sat, NaHCO₃ (30 mL×3), the combined organic layers was dried by Na₂SO₄, filtered and the filtrate was concentrated to get a residue. The residue was purified by column chromatography (SiO₂, Petroleum ether/Ethyl acetate=20/1 to 5/1) to give compound benzyl 3-(tert-butoxycarbonylamino)-4-oxo-piperidine-1-carboxylate (5 g, 14.4 mmol, 93.13% yield) was obtained as a yellow solid.

To a solution of benzyl 3-(tert-butoxycarbonylamino)-4-oxo-piperidine-1-carboxylate (2.5 g, 7.2 mmol, 1 eq) in MeOH (20 mL) was added NH₄OAc (5.5 g, 71.8 mmol, 10 eq) and NaBH₃CN (1.8 g, 28.7 mmol, 4 eq). The mixture was stirred at 25° C. for 12 hr. LC-MS showed desired compound was detected. The mixture was quenched by NaHCO₃ 50 mL and then extracted by ethyl acetate (50 mL×3), the combined organic layers was dried by Na₂SO₄, filtered and the filtrate was concentrated to get a residue. The residue was purified by column chromatography (SiO₂, Petroleum ether/Ethyl acetate=3/1 to 0/1) to give Compound benzyl 4-amino-S-(tert-butoxycarbonylamino)piperidine-1-carboxylate (4.05 g, 11.59 mmol, 80.76% yield) was obtained as a yellow oil.

To a solution of 4-(methylamino)-2-methylsulfanyl-pyrimidine-5-carbaldehyde (1.0 g, 5.7 mmol, 1 eq) benzyl 4-amino-S-(tert-butoxycarbonylamino)piperidine-1-carboxylate (2 g, 5.7 mmol, 1 eq) in MeOH (20 mL) was added AcOH (1.0 g, 17.2 mmol, 982.06 uL, 3 eq) . The mixture was stirred at 50° C. for 12 hr, then NaBH₃CN (1.1 g, 17.2 mmol, 3 eq) was added, the mixture was stirred at 50° C. for 2 hr. LC-MS showed desired compound was detected. The mixture was quenched by NaHCO₃ 10 mL and then extracted by ethyl acetate (10 mL×3), the combined organic layers was dried by Na₂SO₄, filtered and the filtrate was concentrated to get a residue. The residue was purified by column chromatography (SiO₂, Petroleum ether/Ethyl acetate=5/1 to 1/1) to give compound benzyl 3-(tert-butoxycarbonylamino)-4-[[4-(methylamino)-2-methylsulfanyl-pyrimidin-5-yl]methylamino]piperidine-1-carboxylate (3.7 g, 7.2 mmol, 62.56% yield) was obtained as a colorless oil.

To a solution of benzyl 3-(tert-butoxycarbonylamino)-4-[[4-(methylamino)-2-methylsulfanyl-pyrimidin-5-yl]methylamino]piperidine-1-carboxylate (3.7 g, 7.2 mmol, 1 eq) in DCM (40 mL) was added TEA (2.90 g, 28.7 mmol, 4.0 mL, 4 eq) the mixture was cooled to 0° C., and then triphosgene (850.1 mg, 2.9 mmol, 0.4 eq) was added. The mixture was stirred at 25° C. for 12 hr. LC-MS showed reactant remained, so more triphosgene (1.1 g, 3.6 mmol, 0.5 eq) was added, the mixture was stirred at 25° C. for 2 hr. LC-MS showed desired compound was detected. The mixture was quenched by Sat. NaHCO₃ 15 ml and then extracted by DCM (5 mL×3), the organic layers was dried by Na₂SO₄, filtered and the filtrate was concentrated to get a residue. The residue was purified by flash silica gel chromatography (12 g Silica Flash Column, Eluent of 0-35% Ethyl acetate/Petroleum ether gradient @ 100 mL/min) to give benzyl 3-(tert-butoxycarbonylamino)-4-(1-methyl-7-methylsulfanyl-2-oxo-4H-pyrimido[4,5-d]pyrimidin-S-yl)piperidine-1-carboxylate (3.01 g, 5.55 mmol, 77.45% yield) as a yellow solid.

To a solution of benzyl 3-(tert-butoxycarbonylamino)-4-(1-methyl-7-methylsulfanyl-2-oxo-4H-pyrimido[4,5-d]pyrimidin-S-yl)piperidine-1-carboxylate (3 g, 5.5 mmol, 1 eq) in DCM (30 mL) was added m-CPBA (2.24 g, 11.1 mmol, 85% purity, 2 eq). The mixture was stirred at 25° C. for 1 hr. LC-MS showed desired compound was detected. The mixture was quenched by sat. Na₂SO₃ 50 mL, then the mixture was extracted by DCM (20 mL×3), the combined organic layers was washed by sat. NaHCO₃ (15 mL×3), then dried by Na₂SO₄, filtered and the filtrate was concentrated to get compound benzyl 3-(tert-butoxycarbonylamino)-4-(1-methyl-7-methylsulfonyl-2-oxo-4H-pyrimido[4,5-d]pyrimidin-S-yl)piperidine-1-carboxylate (2.7 g, crude) was obtained as a yellow oil.

To a solution of benzyl 3-(tert-butoxycarbonylamino)-4-(1-methyl-7-methylsulfonyl-2-oxo-4H-pyrimido[4,5-d]pyrimidin-S-yl)piperidine-1-carboxylate (300 mg, 522.1 umol, 1 eq) 4-(4-methylpiperazin-1-yl)aniline (109.84 mg, 574.3 umol, 1.1 eq) in dioxane (2 mL) was added TFA (71.4 mg, 626.5 umol, 46.4 uL, 1.2 eq). The mixture was stirred at 100° C. for 12 hr. LC-MS showed desired compounds were detected. The mixture was concentrated to get a residue. The residue was purified by prep-HPLC (TFA condition; column: Phenomenex luna C18 100×40 mm×5 um; mobile phase: [water(TFA)-CAN]) to give benzyl rel-(3S,4S)—S-(tert-butoxycarbonylamino)-4-[1-methyl-7-[4-(4-methylpiperazin-1-yl)anilino]-2-oxo-4H-pyrimido[4,5-d]pyrimidin-S-yl]piperidine-1-carboxylate (240 mg, 350.0 umol, 22.34% yield) as a white solid; and benzyl rel-(3R,4S)—S-((tert-butoxycarbonyl)amino)-4-(1-methyl-7-((4-(4-methylpiperazin-1-yl)phenyl)amino)-2-oxo-1,4-dihydropyrimido[4,5-d]pyrimidin-S(2H)-yl)piperidine-1-carboxylate (230 mg, 335.0 umol, 20.4% yield) as a white solid.

To a mixture of Pd/C (200 mg, 291.6 umol, 10% purity), in THE (10 mL) was added benzyl rel-(3S,4S)—S-(tert-butoxycarbonylamino)-4-[1-methyl-7-[4-(4-methylpiperazin-1-yl)anilino]-2-oxo-4H-pyrimido[4,5-d]pyrimidin-S-yl]piperidine-1-carboxylate (200.0 mg, 291.6 umol, 1 eq) then degassed and purged with H₂ for 3 times, and then the mixture was stirred at 25° C. for 1 hr under H₂ atmosphere (15 psi). LC-MS showed desired compound was detected. The mixture was filtered and the filtrate was concentrated to get compound tert-butyl rel-(3S,4S)—N-[4-[1-methyl-7-[4-(4-methylpiperazin-1-yl)anilino]-2-oxo-4H-pyrimido[4,5-d]pyrimidin-S-yl]-S-piperidyl]carbamate (156 mg, 282.8 umol, 96.96% yield) as an orange solid.

To a solution of tert-butyl rel-(3S,4S)—N-[4-[1-methyl-7-[4-(4-methylpiperazin-1-yl)anilino]-2-oxo-4H-pyrimido[4,5-d]pyrimidin-S-yl]-S-piperidyl]carbamate (156.0 mg, 282.8 umol, 1 eq) in THE (2 mL) and H₂O (0.2 mL) was added NaHCO₃ (71.3 mg, 848.3 umol, 33.0 uL, 3 eq), then prop-2-enoyl chloride (25.6 mg, 282.8 umol, 23.1 uL, 1 eq) was added, then the mixture was stirred at 25° C. for 1 hr. LC-MS showed desired compound was detected.

The mixture was concentrated to get a residue. The residue was purified by prep-HPLC (TFA condition; column: Phenomenex Luna C18 100×30 mm×5 um; mobile phase: [water(TFA)-ACN];B %: 1%-30%, 8 min) to give compound tert-butyl rel-(3S,4S)—N-[4-[1-methyl-7-[4-(4-methylpiperazin-1-yl)anilino]-2-oxo-4H-pyrimido[4,5-d]pyrimidin-S-yl]-1-prop-2-enoyl-S-piperidyl]carbamate (20 mg, 33.0 umol, 11.68% yield) as a pale yellow solid.

To a solution of tert-butyl rel-(3S,4S)—N-[4-[1-methyl-7-[4-(4-methylpiperazin-1-yl)anilino]-2-oxo-4H-pyrimido[4,5-d]pyrimidin-S-yl]-1-prop-2-enoyl-S-piperidyl]carbamate (14 mg, 23.11 umol, 1 eq) in DCM (3 mL) was added TFA (0.2 mL). The mixture was stirred at 25° C. for 1 hr. LC-MS showed desired compound was detected. The mixture was concentrated to get a residue. The residue was purified by prep-HPLC (TFA condition; column: Phenomenex Luna C18 100×30 mm×5 um;mobile phase: [water(TFA)-ACN];B %: 1%-25%, 8 min) to give rel-(3S,4S)—S-(3-amino-1-prop-2-enoyl-4-piperidyl)-1-methyl-7-[4-(4-methylpiperazin-1-yl)anilino]-4H-pyrimido[4,5-d]pyrimidin-2-one (6 mg, 9.68 umol, 41.90% yield, 100% purity, TFA) as a white solid.

### Procedure for Preparation of Compound 148

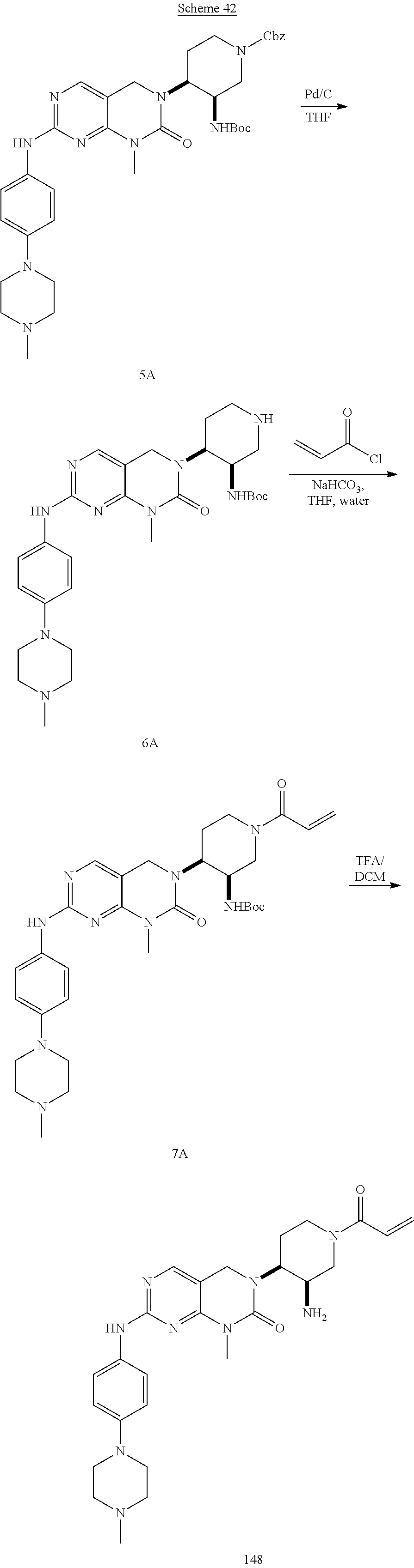

To a mixture of Pd/C (200 mg, 335.4 umol, 10% purity) in THE (10 mL) was added benzyl rel-(3R,4S)—S-(tert-butoxycarbonylamino)-4-[1-methyl-7-[4-(4-methylpiperazin-1-yl)anilino]-2-oxo-4H-pyrimido[4,5-d]pyrimidin-S-yl]piperidine-1-carboxylate (230 mg, 335.4 umol, 1 eq) then degassed and purged with H₂ for 3 times, and then the mixture was stirred at 25° C. for 1 hr under H₂ atmosphere (15 psi). LC-MS showed desired compound was detected. The mixture was filtered and the filtrate was concentrated to get compound tert-butyl rel-(3R,4S)—N-[4-[1-methyl-7-[4-(4-methylpiperazin-1-yl)anilino]-2-oxo-4H-pyrimido[4,5-d]pyrimidin-S-yl]-S-piperidyl]carbamate (140 mg, crude) as a yellow solid.

To a solution of tert-butyl rel-(3R,4S)—N-[4-[1-methyl-7-[4-(4-methylpiperazin-1-yl)anilino]-2-oxo-4H-pyrimido[4,5-d]pyrimidin-S-yl]-S-piperidyl]carbamate (140.0 mg, 253.8 umol, 1 eq) in THE (1.5 mL) and H₂O (0.1 mL) was added NaHCO₃ (63.95 mg, 761.31 umol, 29.61 uL, 3 eq), then prop-2-enoyl chloride (23.0 mg, 253.8 umol, 20.7 uL, 1 eq) was added, then the mixture was stirred at 25° C. for 1 hr. LC-MS showed desired compound was detected. The mixture was concentrated to get a residue. The residue was purified by prep-HPLC (TFA condition; column: Phenomenex luna C18 100×40 mm×5 um; mobile phase: [water(TFA)-ACN];B %: 1%-30%, 8 min) to give compound tert-butyl rel-(3R,4S)—N-[4-[1-methyl-7-[4-(4-methylpiperazin-1-yl)anilino]-2-oxo-4H-pyrimido[4,5-d]pyrimidin-S-yl]-1-prop-2-enoyl-S-piperidyl]carbamate (100 mg, 165.09 umol, 65.06% yield) as a pale yellow oil.

To a solution of tert-butyl rel-(3R,4S)—N-[4-[1-methyl-7-[4-(4-methylpiperazin-1-yl)anilino]-2-oxo-4H-pyrimido[4,5-d]pyrimidin-S-yl]-1-prop-2-enoyl-S-piperidyl]carbamate (80.0 mg, 132.1 umol, 1 eq) in DCM (3 mL) was added TFA (0.2 mL). The mixture was stirred at 25° C. for 1 hr. LC-MS showed desired compound was detected. The mixture was concentrated to get a residue. The residue was purified by prep-HPLC (TFA condition; column: Phenomenex Luna C18 100×30 mm×5 um;mobile phase: [water(TFA)-ACN];B %: 1%-25%, 8 min) to give rel-(3R,4S)—S-(3-amino-1-prop-2-enoyl-4-piperidyl)-1-methyl-7-[4-(4-methylpiperazin-1-yl)anilino]-4H-pyrimido[4,5-d]pyrimidin-2-one (63 mg, 101.67 umol, 76.98% yield, 100% purity, TFA) as a yellow solid.

### Procedure for Preparation of Compound 142

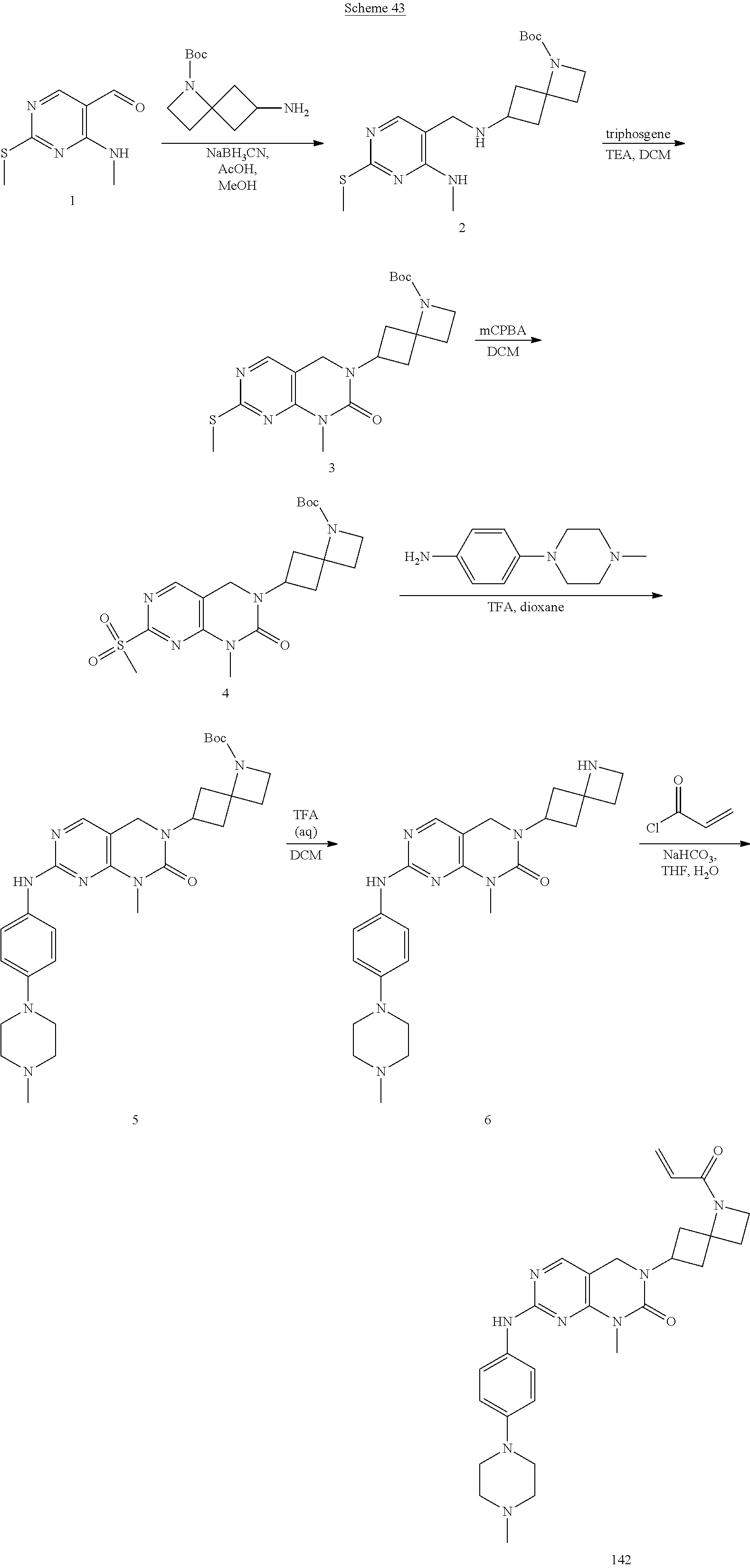

To a solution of 4-(methylamino)-2-methylsulfanyl-pyrimidine-5-carbaldehyde (431.5 mg, 2.3 mmol, 1 eq), tert-butyl 6-amino-1-azaspiro[3.3]heptane-1-carboxylate (500 mg, 2.3 mmol, 1 eq) in MeOH (10 mL) was added AcOH (848.6 mg, 14.1 mmol, 808.2 uL, 6 eq) to adjust pH=5 and the mixture was stirred at 25° C. for 2 hr. NaBH₃CN (444.0 mg, 7.0 mmol, 3 eq) was added to the mixture. The mixture was stirred at 25° C. for 12 hr. LC-MS showed desired mass was detected. The reaction mixture was concentrated under reduced pressure to remove solvent. The residue was diluted with NaHCO₃ 30 mL and extracted with dichloromethane (30 mL×3). The combined organic layers were washed with brine (30 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 100 mL/min) to give tert-butyl 6-[[4-(methylamino)-2-methylsulfanyl-pyrimidin-5-yl]methylamino]-1-azaspiro[3.3]heptane-1-carboxylate (425 mg, 1.0 mmol, 80.7% yield, 95% purity) as colorless oil.

To a solution of tert-butyl 6-[[4-(methylamino)-2-methylsulfanyl-pyrimidin-5-yl]methyla mino]-1-azaspiro[3.3]heptane-1-carboxylate (425 mg, 1.1 mmol, 1 eq) in DCM (5 mL) was ad ded DIEA (434.1 mg, 3.3 mmol, 585.1 uL, 3 eq) and bis(trichloromethyl) carbonate (265.8 m g, 895.8 umol, 0.8 eq) at 0° C. The mixture was stirred at 25° C. for 1 hr. LC-MS showed desi red mass was detected. The residue was diluted with NaHCO₃ 30 mL and extracted with Dic hloromethane (15 mL×3). The combined organic layers were washed with brine (30 mL×1), dried over Na₂SO₄, filtered and concentrated under reduced pressure to give tert-butyl 6-(1-methyl-7-methylsulfanyl-2-oxo-4H-pyrimido[4,5-d]pyrimidin-S-yl)-1-azaspiro[3.3]heptane-1-c arboxylate (450 mg, crude) as yellow oil.

To a solution of tert-butyl 6-(1-methyl-7-methylsulfanyl-2-oxo-4H-pyrimido[4,5-d]pyrimidin-S-yl)-1-azaspiro[3.3]heptane-1-carboxylate (450 mg, 1.1 mmol, 1 eq) in DCM (5 mL) was added m-CPBA (337.9 mg, 1.6 mmol, 85% purity, 1.5 eq) at 0° C. The mixture was stirred at 0° C. for 2 hr. LC-MS showed desired mass was detected. The residue was diluted with Na₂SO₃ 10 mL and extracted with dichloromethane (20 mL×3). The combined organic layers were washed with NaHCO₃ (20 mL×3), dried over Na₂SO₄, filtered and concentrated under reduced pressure to give tert-butyl 6-(1-methyl-7-methylsulfonyl-2-oxo-4H-pyrimido[4,5-d]pyrimidin-S-yl)-1-azaspiro[3.3]heptane-1-carboxylate (480 mg, crude) as yellow oil.

To a solution of tert-butyl 6-(1-methyl-7-methylsulfonyl-2-oxo-4H-pyrimido[4,5-d]pyrimidin-S-yl)-1-azaspiro[3.3]heptane-1-carboxylate (480 mg, 1.1 mmol, 1 eq) in dioxane (5 mL) was added TFA (187.6 mg, 1.6 mmol, 121.8 uL, 1.5 eq) and 4-(4-methylpiperazin-1-yl)aniline (314.7 mg, 1.6 mmol, 1.5 eq). The mixture was stirred at 120° C. for 3 hr. LC-MS showed desired mass was detected. The reaction mixture was concentrated under reduced pressure to remove solvent. The residue was purified by prep-HPLC (FA condition, column: Phenomenex C18(75×30 mm×3 um);mobile phase: [water(FA)-ACN];B %: 1%-30%, 8 min) to give tert-butyl 6-[1-methyl-7-[4-(4-methylpiperazin-1-yl)anilino]-2-oxo-4H-pyrimido[4,5-d]pyrimidin-S-yl]-1-azaspiro[3.3]heptane-1-carboxylate (250 mg, 455.6 umol, 41.5% yield) as a white solid.

To a solution of tert-butyl 6-[1-methyl-7-[4-(4-methylpiperazin-1-yl)anilino]-2-oxo-4H-pyrimido[4,5-d]pyrimidin-S-yl]-1-azaspiro[3.3]heptane-1-carboxylate (127 mg, 231.4 umol, 1 eq) in DCM (2 mL) was added TFA (395.8 mg, 3.4 mmol, 257.0 uL, 15 eq). The mixture was stirred at 20° C. for 2 hr. LC-MS showed desired mass was detected. The reaction mixture was concentrated under reduced pressure to remove solvent to give 3-(1-azaspiro[3.3]heptan-6-yl)-1-methyl-7-[4-(4-methylpiperazin-1-yl)anilino]-4H-pyrimido[4,5-d]pyrimidin-2-one (130 mg, 231.0 umol, TFA) as a colorless oil.

To a solution of 3-(1-azaspiro[3.3]heptan-6-yl)-1-methyl-7-[4-(4-methylpiperazin-1-yl)anilino]-4H-pyrimido[4,5-d]pyrimidin-2-one (130 mg, 231.0 umol, 1 eq, TFA) and NaHCO₃ (58.2 mg, 693.2 umol, 26.9 uL, 3 eq) in THE (0.5 mL) and H₂O (0.5 mL) was added and prop-2-enoyl chloride (18.8 mg, 207.9 umol, 16.9 uL, 0.9 eq) at 0° C. The mixture was stirred at 0° C. for 1 hr. LC-MS showed desired mass was detected. The reaction mixture was concentrated under reduced pressure to remove solvent. The residue was purified by prep-HPLC (FA condition, column: Phenomenex C18 75×30 mm×3 um;mobile phase: [water(FA)-ACN];B %: 1%-25%, 8 min) to give 1-methyl-7-[4-(4-methylpiperazin-1-yl)anilino]-S-(1-prop-2-enoyl-1-azaspiro[3.3]heptan-6-yl)-4H-pyrimido[4,5-d]pyrimidin-2-one (30 mg, 59.6 umol, 25.8% yield, 100% purity) as a white solid.

### Procedure for Preparation of Compound 138

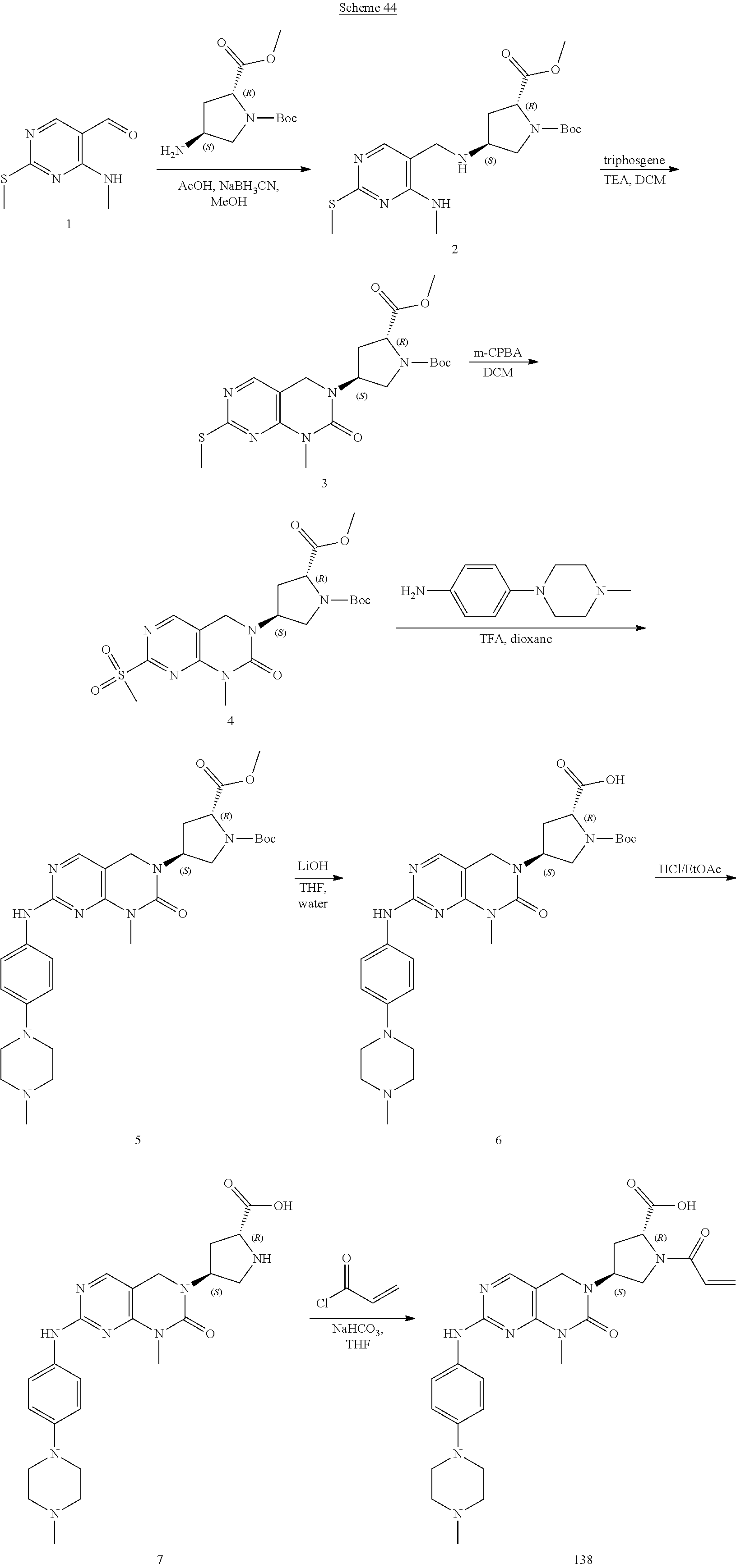

To a solution of 4-(methylamino)-2-methylsulfanyl-pyrimidine-5-carbaldehyde (500 mg, 2.7 mmol, 1 eq) and O1-tert-butyl 02-methyl 4-aminopyrrolidine-1,2-dicarboxylate (666.6 mg, 2.7 mmol, 1 eq) in MeOH (20 mL) was added AcOH (16.3 mg, 272.8 umol, 15.6 uL, 0.1 eq) to make pH=5, the mixture was stirred at 60° C. for 1 hr, then NaBH₃CN (1.0 g, 16.3 mmol, 6 eq) was added in the mixture. The mixture was stirred at 60° C. for 12 hr. TLC indicated 4-(methylamino)-2-methylsulfanyl-pyrimidine-5-carbaldehyde was consumed completely and one new spot formed. The mixture was concentrated under vacuum. The residue was diluted with 50 mL H₂O and extracted with Ethyl acetate (30 mL×3). The combined organic phase was dried with anhydrous Na₂SO₄, and the mixture was filtered and the filtrate was concentrated under vacuum. The residue was purified by flash silica gel chromatography (20 g Silica Flash Column, Eluent of 0-100% Ethyl acetate/Petroleum ether gradient @ 100 mL/min) to give 01-tert-butyl 02-methyl 4-[[4-(methylamino)-2-methylsulfanyl-pyrimidin-5-yl]methylamino] pyrrolidine-1,2-dicarboxylate (1.9 g, 4.6 mmol, 84.6% yield) as a yellow oil.

To a solution of O1-tert-butyl 02-methyl 4-[[4-(methylamino)-2-methylsulfanyl-pyrimidin-5-yl]methylamino]pyrrolidine-1,2-dicarboxylate (2.9 g, 7.0 mmol, 1 eq) in DCM (30 mL) was added DIEA (2.7 g, 21.1 mmol, 3.6 mL, 3 eq) and bis(trichloromethyl) carbonate (1.8 g, 6.3 mmol, 0.9 eq). The mixture was stirred at 0° C. for 2 hr. LC-MS showed O1-tert-butyl 02-methyl 4-[[4-(methylamino)-2-methylsulfanyl-pyrimidin-5-yl]methylamino]pyrrolidine-1,2-dicarboxylate was consumed completely and desired mass was detected. The residue was diluted with NaHCO₃ 50 mL and extracted with Dichloromethane 30 mL (10 mL×3). The combined organic layers were washed with brine 20 mL (10 mL×2), dried over Na₂SO₄, filtered and concentrated under reduced pressure to give 01-tert-butyl 02-methyl 4-(1-methyl-7-methylsulfanyl-2-oxo-4H-pyrimido[4,5-d]pyrimidin-S-yl)pyrrolidine-1,2-dicarboxylate (2.4 g, crude) as yellow oil.

To a solution of O1-tert-butyl 02-methyl 4-(1-methyl-7-methylsulfanyl-2-oxo-4H-pyrimido[4,5-d]pyrimidin-S-yl)pyrrolidine-1,2-dicarboxylate (2.4 g, 5.4 mmol, 1 eq) in DCM (20 mL) was added m-CPBA (1.4 g, 7.1 mmol, 85% purity, 1.3 eq) at 0° C. The mixture was stirred at 0° C. for 2 hr. LC-MS showed desired mass was detected. The reaction mixture was partitioned between Na₂SO₃ 50 mL and Dichloromethane (50×3 mL). The organic phase was separated, washed with NaHCO₃ (50 mL×3), dried over Na₂SO₄, filtered and concentrated under reduced pressure to give a residue. The residue was purified by flash silica gel chromatography (20 g Silica Flash Column, Eluent of 0-100% Ethyl acetate/Petroleum ether gradient @ 100 mL/min) to give 01-tert-butyl 02-methyl (2R,4S)-4-(1-methyl-7-methylsulfonyl-2-oxo-4H-pyrimido[4,5-d]pyrimidin-S-yl)pyrrolidine-1,2-dicarboxylate (1.4 g, 1.4 mmol, 27.1% yield, 50% purity) as a yellow solid.

To a solution of 01-tert-butyl 02-methyl (2R,4S)-4-(1-methyl-7-methylsulfonyl-2-oxo-4H-pyrimido[4,5-d]pyrimidin-S-yl)pyrrolidine-1,2-dicarboxylate (350 mg, 745.4 umol, 1 eq) in dioxane (3 mL) was added TFA (127.5 mg, 1.1 mmol, 82.7 uL, 1.5 eq) and 4-(4-methylpiperazin-1-yl)aniline (213.8 mg, 1.1 mmol, 1.5 eq). The mixture was stirred at 120° C. for 2 hr. LC-MS showed desired mass was detected. The reaction mixture was concentrated under reduced pressure to remove solvent. The residue was purified by prep-HPLC (FA condition, column: Phenomenex Luna C18 100×30 mm×5 um;mobile phase: [water(FA)-ACN];B %: 5%-35%, 8 min) to give 01-tert-butyl 02-methyl (2R,4S)-4-[1-methyl-7-[4-(4-methylpiperazin-1-yl)anilino]-2-oxo-4H-pyrimido[4,5-d]pyrimidin-S-yl]pyrrolidine-1,2-dicarboxylate (280 mg, 482.2 umol, 64.6% yield) as a yellow solid.

To a solution of 01-tert-butyl 02-methyl (2R,4S)-4-[1-methyl-7-[4-(4-methylpiperazin-1-yl)anilino]-2-oxo-4H-pyrimido[4,5-d]pyrimidin-S-yl]pyrrolidine-1,2-dicarboxylate (280.0 mg, 482.2 umol, 1 eq) in THE (1 mL)/MeOH (0.3 mL)/H₂O (0.1 mL) was added LiGH (69.2 mg, 2.8 mmol, 6 eq). The mixture was stirred at 75° C. for 4 hr. LC-MS showed desired mass was detected. The reaction mixture was concentrated under reduced pressure to remove solvent. The residue was added 0.5M HCl to tune the pH-2, and then diluted with NH4Cl solution 30 mL and extracted with Dichloromethane 30 mL (10 mL×3). The combined organic layers were washed with brine 20 mL (10 mL×2), dried over Na₂SO₄, filtered and concentrated under reduced pressure to give (2R,4S)-1-tert-butoxycarbonyl-4-[1-methyl-7-[4-(4-methylpiperazin-1-yl)anilino]-2-oxo-4H-pyrimido[4,5-d]pyrimidin-S-yl]pyrrolidine-2-carboxylic acid (250 mg, crude) as a white solid.

A solution of (2R,4S)-1-tert-butoxycarbonyl-4-[1-methyl-7-[4-(4-methylpiperazin-1-yl)anilino]-2-oxo-4H-pyrimido[4,5-d]pyrimidin-S-yl]pyrrolidine-2-carboxylic acid (250 mg, 441.1 umol, 1 eq) in HCl/EtOAc (2 mL, 4 M) was stirred at 20° C. for 1 hr. LC-MS showed desired mass was detected. The reaction mixture was concentrated under reduced pressure to give (2R, 4S)-4-[1-methyl-7-[4-(4-methylpiperazin-1-yl)anilino]-2-oxo-4H-pyrimido[4,5-d]pyrimidin-S-yl]pyrrolidine-2-carboxylic acid (166 mg, crude, HCl) as a white solid.

To a solution of (2R,4S)-4-[1-methyl-7-[4-(4-methylpiperazin-1-yl)anilino]-2-oxo-4H-pyrimido[4,5-d]pyrimidin-S-yl]pyrrolidine-2-carboxylic acid (100 mg, 198.8 umol, 1 eq, HCl) and NaHCO₃ (50.1 mg, 596.4 umol, 23.2 uL, 3 eq) in THE (1 mL) was added prop-2-enoyl chloride (14.4 mg, 159.0 umol, 12.9 uL, 0.8 eq) at 0° C. The mixture was stirred at 0° C. for 15 min. LC-MS showed desired compound was detected. The reaction mixture was concentrated under reduced pressure to remove solvent. The residue was purified by prep-HPLC (FA condition, column: Phenomenex Luna C18 100×30 mm×5 um;mobile phase: [water(FA)-ACN];B %: 1%-15%, 8 min) to give (2R,4S)-4-[1-methyl-7-[4-(4-methylpiperazin-1-yl)anilino]-2-oxo-4H-pyrimido[4,5-d]pyrimidin-S-yl]-1-prop-2-enoyl-pyrrolidine-2-carboxylic acid (17 mg, 31.7 umol, 15.9% yield, 97.1% purity) as a yellow solid.

### Procedure for Preparation of Compound 136

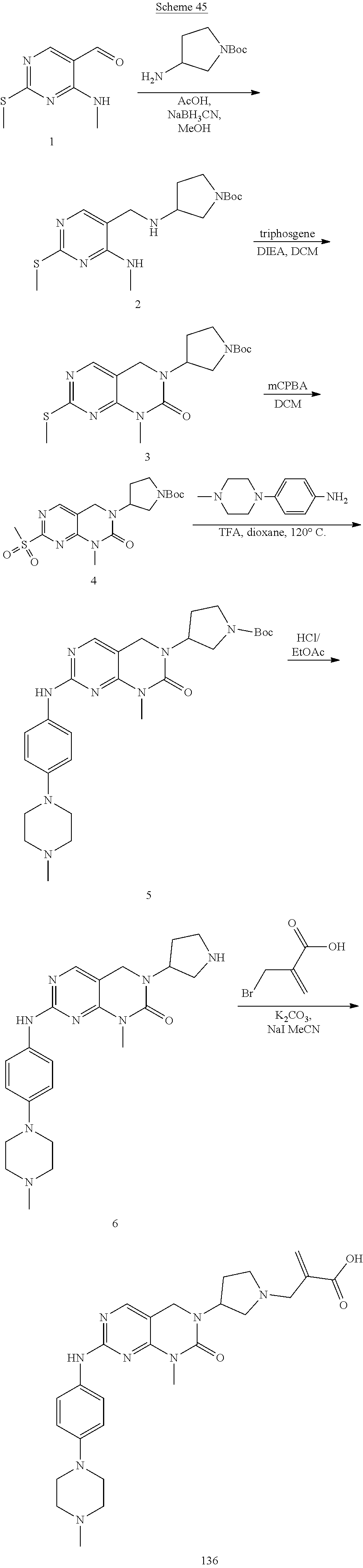

To a solution of 4-(methylamino)-2-methylsulfanyl-pyrimidine-5-carbaldehyde (1 g, 5.4 mmol, 1 eq) in MeOH (10 mL) was added tert-butyl 3-aminopyrrolidine-1-carboxylate (1.0 g, 5.4 mmol, 1 eq). Then CH₃COOH (1.3 g, 21.8 mmol, 1.2 mL, 4 eq) was added to adjust pH to 5-6. The mixture was stirred at 65° C. for 12 hr. NaBH₃CN (1.0 g, 16.3 mmol, 3 eq) was added, the mixture was stirred at 65° C. for 2 hr. LC-MS showed desired mass was detected. The reaction mixture was concentrated under reduced pressure to remove solvent. The reaction mixture was partitioned between H₂O 50 mL and dichloromethane (50×3 mL). The organic phase was separated, washed with brine (50 mL×3), dried over Na₂SO₄, filtered and concentrated under reduced pressure to give a residue. The residue was purified by flash silica gel chromatography (20 g Silica Flash Column, Eluent of 0-80% Ethyl acetate/Petroleum ether gradient @ 100 mL/min) to give tert-butyl 3-[[4-(methylamino)-2-methylsulfanyl-pyrimidin-5-yl]methyl amino]pyrrolidine-1-carboxylate (2 g, 4.9 mmol, 90.1% yield, 87% purity) as a white solid.

To a solution of tert-butyl 3-[[4-(methylamino)-2-methylsulfanyl-pyrimidin-5-yl]methylamino]pyrrolidine-1-carboxylate (1 g, 2.8 mmol, 1 eq) in DCM (20 mL) was added DIEA (1.1 g, 8.4 mmol, 1.4 mL, 3 eq) and bis(trichloromethyl) carbonate (755.5 mg, 2.5 mmol, 0.9 eq) at 0° C. The mixture was stirred at 0° C. for 2 hr. LC-MS showed desired mass was detected. The residue was diluted with NaHCO₃ 30 mL and extracted with dichloromethane 60 mL (20 mL×3). The combined organic layers were washed with brine 40 mL (20 mL×2), dried over Na₂SO₄, filtered and concentrated under reduced pressure to give tert-butyl 3-(1-methyl-7-methylsulfanyl-2-oxo-4H-pyrimido[4,5-d]pyrimidin-S-yl)pyrrolidine-1-carboxylate (2.2 g, crude) as brown oil.

To a solution of tert-butyl 3-(1-methyl-7-methylsulfanyl-2-oxo-4H-pyrimido[4,5-d]pyrimidin-S-yl)pyrrolidine-1-carboxylate (1.5 g, 3.9 mmol, 1 eq) in DCM (10 mL) was added m-CPBA (963.0 mg, 4.7 mmol, 85% purity, 1.2 eq) at 0° C. The mixture was stirred at 20° C. for 5 hr. LC-MS showed desired mass was detected. The reaction mixture was partitioned between Na₂SO₃ 50 mL and dichloromethane (50×3 mL). The organic phase was separated, washed with NaHCO₃ (50 mL×3), dried over Na₂SO₄, filtered and concentrated under reduced pressure to give a residue. The residue was purified by flash silica gel chromatography (20 g Silica Flash Column, Eluent of 0-100% Ethyl acetate/Petroleum ether gradient @100 mL/min) to give tert-butyl 3-(1-methyl-7-methylsulfonyl-2-oxo-4H-pyrimido [4, 5-d] pyrimidin-S-yl) pyrrolidine-1-carboxylate (300 mg, 729.0 umol, 18.4% yield) as a yellow solid.

To a solution of tert-butyl 3-(1-methyl-7-methylsulfonyl-2-oxo-4H-pyrimido[4,5-d]pyrimidin-S-yl)pyrrolidine-1-carboxylate (280 mg, 680.4 umol, 1 eq) in dioxane (2 mL) was added TFA (116.3 mg, 1.0 mmol, 75.5 uL, 1.5 eq) and 4-(4-methylpiperazin-1-yl)aniline (156.1 mg, 816.5 umol, 1.2 eq). The mixture was stirred at 120° C. for 2 hr. LC-MS showed desired compound was detected. The reaction mixture was concentrated under reduced pressure to remove solvent. The residue was purified by prep-HPLC (FA condition, column: Phenomenex Luna C18 100×30 mm×5 um;mobile phase: [water(FA)-ACN]; B %: 1%-30%, 8 min) to give tert-butyl 3-[1-methyl-7-[4-(4-methylpiperazin-1-yl)anilino]-2-oxo-4H-pyrimido[4,5-d]pyrimidin-S-yl]pyrrolidine-1-carboxylate (60 mg, 114.8 umol, 8.4% yield) as yellow oil.

A solution of tert-butyl 3-[1-methyl-7-[4-(4-methylpiperazin-1-yl)anilino]-2-oxo-4H-pyrimido[4,5-d]pyrimidin-S-yl]pyrrolidine-1-carboxylate (60 mg, 114.8 umol, 1 eq) in HCl/EtOAc (2 mL, 4 M) was stirred at 20° C. for 0.5 hr. LC-MS showed desired compound was detected. The reaction mixture was concentrated under reduced pressure to remove solvent to give 1-methyl-7-[4-(4-methylpiperazin-1-yl)anilino]-S-pyrrolidin-S-yl-4H-pyrimido[4,5-d]pyrimidin-2-one (50 mg, crude) as a white solid.

To a solution of 1-methyl-7-[4-(4-methylpiperazin-1-yl)anilino]-S-pyrrolidin-S-yl-4H-pyrimido[4,5-d]pyrimidin-2-one (40.9 mg, 89.1 umol, 1 eq, HCl) and TEA (27.0 mg, 267.3 umol, 37.2 uL, 3 eq) in DCM (0.5 mL) was added a solution of 2-(bromomethyl)prop-2-enoic acid (13.2 mg, 80.2 umol, 0.9 eq) in DCM (0.5 mL). The mixture was stirred at 20° C. for 2 hr. LC-MS showed desired mass was detected. The reaction mixture was concentrated under reduced pressure to remove solvent. The residue was purified by prep-HPLC (FA condition, column: Phenomenex Luna C18 75×30 mm×3 um;mobile phase: [water(FA)-ACN];B %: 1%-20%, 8 min) to give 2-[[3-[1-methyl-7-[4-(4-methylpiperazin-1-yl)anilino]-2-oxo-4H-pyrimido[4,5-d]pyrimidin-S-yl]pyrrolidin-1-yl]methyl]prop-2-enoic acid (11.2 mg, 22.1 umol, 24.8% yield, 100% purity) as a yellow solid.

### Procedure for Preparation of Compound 137

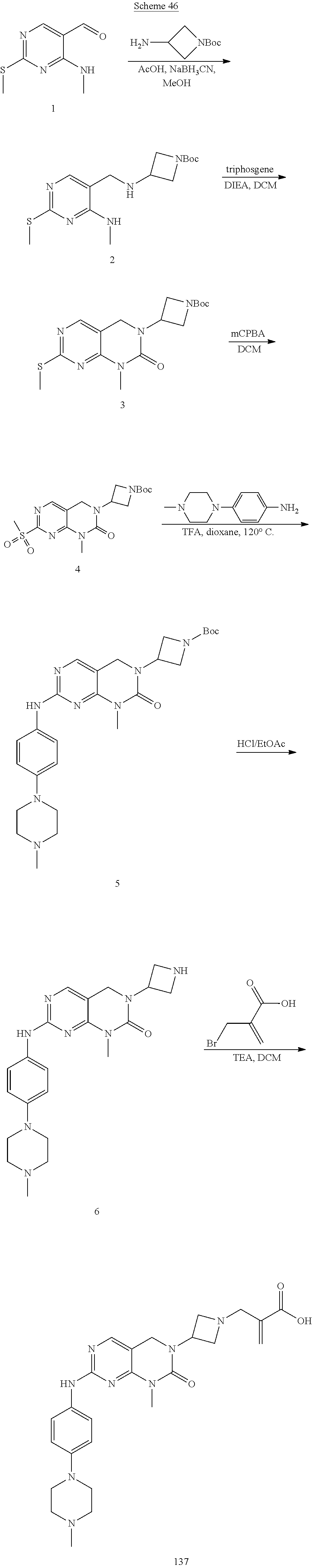

To a solution of 4-(methylamino)-2-methylsulfanyl-pyrimidine-5-carbaldehyde (500 mg, 2.7 mmol, 1 eq) and tert-butyl 3-aminoazetidine-1-carboxylate (469.9 mg, 2.7 mmol, 1 eq) in MeOH (20 mL) was added AcOH (16.3 mg, 272.8 umol, 15.6 uL, 0.1 eq) to make pH=5, the mixture was stirred at 60° C. for 1 hr. Then NaBH₃CN (1.0 g, 16.3 mmol, 6 eq) was added to the mixture. The mixture was stirred at 60° C. for 12 hr. TLC indicated 4-(methylamino)-2-methylsulfanyl-pyrimidine-5-carbaldehyde was consumed completely and one new spot formed. The mixture was concentrated under vacuum. The residue was diluted with 50 mL H₂O and extracted with Ethyl acetate (30 mL×3). The combined organic phase was dried with anhydrous Na₂SO₄, and the mixture was filtered and the filtrate was concentrated under vacuum. The residue was purified by flash silica gel chromatography (12 g Silica Flash Column, Eluent of 0-100% Ethyl acetate/Petroleum ether gradient @ 100 mL/min) to give tert-butyl 3-[[4-(methylamino)-2-methylsulfanyl-pyrimidin-5-yl]methylamino]azetidine-1-carboxylate (1.6 g, 4.7 mmol, 86.3% yield) as a yellow oil.

To a solution of tert-butyl 3-[[4-(methylamino)-2-methylsulfanyl-pyrimidin-5-yl]methylamino]azetidine-1-carboxylate (3.5 g, 10.5 mmol, 1 eq) in DCM (30 mL) was added DIEA (4.0 g, 31.5 mmol, 5.5 mL, 3 eq) and bis(trichloromethyl) carbonate (2.8 g, 9.4 mmol, 0.9 eq) at 0° C. The mixture was stirred at 0° C. for 2 hr. LC-MS showed desired mass was detected. The residue was diluted with NaHCO₃ 50 mL and extracted with Dichloromethane 30 mL (10 mL×3). The combined organic layers were washed with brine 20 mL (10 mL×2), dried over Na₂SO₄, filtered and concentrated under reduced pressure to give a residue.

The residue was purified by flash silica gel chromatography (Silica Flash Column, Eluent of 0-100% Ethyl acetate/Petroleum ether gradient @ 100 mL/min) to give tert-butyl 3-(1-methyl-7-methylsulfanyl-2-oxo-4H-pyrimido[4,5-d]pyrimidin-S-yl)azetidine-1-carboxylate (650 mg, 889.3 umol, 8.4% yield, 50% purity) as a white solid.

To a solution of tert-butyl 3-(1-methyl-7-methylsulfanyl-2-oxo-4H-pyrimido[4,5-d]pyrimidin-S-yl)azetidine-1-carboxylate (650 mg, 1.7 mmol, 1 eq) in DCM (10 mL) was added m-CPBA (541.6 mg, 2.6 mmol, 85% purity, 1.5 eq) at 0° C. The mixture was stirred at 0° C. for 2 hr. LC-MS showed desired mass was detected. The reaction mixture was partitioned between Na₂SO₃ 50 mL and Dichloromethane (50×3 mL). The organic phase was separated, washed with NaHCO₃ (50 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 100 mL/min) to give tert-butyl 3-(1-methyl-7-methylsulfonyl-2-oxo-4H-pyrimido[4,5-d]pyrimidin-S-yl)azetidine-1-carboxylate (230 mg, 289.3 umol, 16.2% yield, 50% purity) as a white solid.

To a solution of tert-butyl 3-(1-methyl-7-methylsulfonyl-2-oxo-4H-pyrimido[4,5-d]pyrimidin-S-yl)azetidine-1-carboxylate (230.0 mg, 578.6 umol, 1 eq) in dioxane (2 mL) was added TFA (98.9 mg, 868.0 umol, 64.2 uL, 1.5 eq) and 4-(4-methylpiperazin-1-yl)aniline (166.0 mg, 868.0 umol, 1.5 eq). The mixture was stirred at 120° C. for 4 hr. LC-MS showed desired mass was detected. The reaction mixture was concentrated under reduced pressure to remove solvent. The residue was purified by prep-HPLC (FA condition, column: Phenomenex Luna C18 100×30 mm×5 um;mobile phase: [water(FA)-ACN];B %: 1%-30%, 8 min) to give tert-butyl 3-[1-methyl-7-[4-(4-methylpiperazin-1-yl)anilino]-2-oxo-4H-pyrimido[4,5-d]pyrimidin-S-yl]azetidine-1-carboxylate (105 mg, 206.4 umol, 35.6% yield) as a white solid.

A solution of tert-butyl 3-[1-methyl-7-[4-(4-methylpiperazin-1-yl)anilino]-2-oxo-4H-pyrimido[4,5-d]pyrimidin-S-yl]azetidine-1-carboxylate (105 mg, 206.4 umol, 1 eq) in HCl/EtOAc (4 M, 1.0 mL, 20 eq) was stirred at 20° C. for 1 hr. LC-MS showed desired mass was detected. The reaction mixture was concentrated under reduced pressure to give 3-(azetidin-S-yl)-1-methyl-7-[4-(4-methylpiperazin-1-yl)anilino]-4H-pyrimido[4,5-d]pyrimidin-2-one (102 mg, crude, HCl) as a white solid.

To a solution of 3-(azetidin-S-yl)-1-methyl-7-[4-(4-methylpiperazin-1-yl)anilino]-4H-pyrimido[4,5-d]pyrimidin-2-one (90 mg, 202.2 umol, 1 eq, HCl) in DCM (1.5 mL) was added TEA (61.4 mg, 606.8 umol, 84.4 uL, 3 eq) to adjust pH to 7-8, and then 2-(bromomethyl)prop-2-enoic acid (30.0 mg, 182.0 umol, 0.9 eq) in DCM (0.5 mL) was added. The mixture was stirred at 20° C. for 1 hr. LC-MS showed desired mass was detected. The reaction mixture was concentrated under reduced pressure to remove solvent. The residue was purified by prep-HPLC (FA condition, column: Phenomenex Luna C18 100×30 mm×5 um;mobile phase: [water(FA)-ACN];B %: 1%-10%, 8 min) to give 2-[[3-[1-methyl-7-[4-(4-methylpiperazin-1-yl)anilino]-2-oxo-4H-pyrimido[4,5-d]pyrimidin-S-yl]azetidin-1-yl]methyl]prop-2-enoic acid (9 mg, 18.0 umol, 8.9% yield, 98.4% purity) as an orange solid.

### Procedure for Preparation of Compound 150

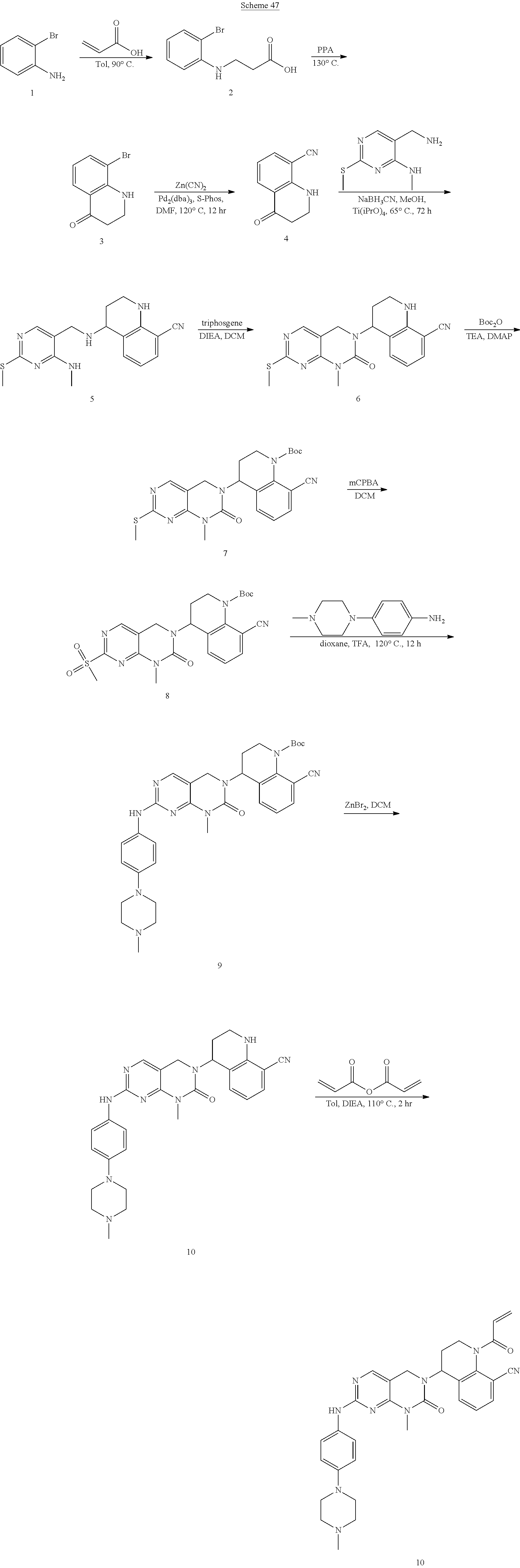

To a solution of 2-bromoaniline (50 g, 40.7 mmol, 1 eq) in Tol. (500 mL) was added acrylic acid (20.5 g, 40.7 mmol, 2.8 mL, 1 eq). The mixture was stirred at 90° C. for 12 hr. LC-MS showed the desired compound was detected. The reaction mixture was concentrated under reduced pressure to give a residue. The residue was purified by flash silica gel chromatography (220 g Silica Flash Column, Eluent of 0-50% Ethyl acetate/Petroleum ether gradient @ 150 mL/min) to give 3-(2-bromoanilino) propanoic acid (25 g, 102.4 mmol, 35.9% yield) as a brown solid.

A mixture of 3-(2-bromoanilino) propanoic acid (30 g, 40.97 mmol, 1 eq) in PPA (300 mL) was stirred at 130° C. for 2 hr. LC-MS showed the desired compound was detected. The reaction mixture was diluted with H₂O (500 mL) and basified the PH=8 with NaOH. Then extracted with dichloromethane (500 mL×3). The combined organic layers were washed with brine (300 mL×1), dried over Na₂SO₄, filtered and concentrated under reduced pressure to give a residue. The residue was purified by flash silica gel chromatography (120 g Silica Flash Column, Eluent of 0-20% Ethyl acetate/Petroleum ether gradient @ 150 mL/min) to give 8-bromo-2,3-dihydro-1 H-quinolin-4-one (31.48 g) as a yellow oil.

To a solution of 8-bromo-2,3-dihydro-1H-quinolin-4-one (6 g, 8.8 mmol, 1 eq) in DMF (60 mL) was added Zn(CN)₂ (9.4 g, 26.5 mmol, 3 eq), Pd₂(dba)₃ (2.4 g, 884.7 umol, 0.1 eq) and s-Phos (1.1 g, 884.7 umol, 0.1 eq) was degassed and purged with N₂ for 3 times. The mixture was stirred at 120° C. for 12 hr under N₂. LC-MS showed the desired compound was detected. The reaction mixture was diluted with H₂O (100 mL) and extracted with ethyl acetate (100 mL×3). The aqueous layer was quenched by NaClO solution, and the combined organic layers were washed with brine (50 mL×2), dried over Na₂SO₄, filtered and concentrated under reduced pressure to give a residue. The residue was purified by flash silica gel chromatography (80 g Silica Flash Column, Eluent of 0-20% Ethyl acetate/Petroleum ether gradient @ 150 mL/min) to give 4-oxo-2,3-dihydro-1 H-quinoline-8-carbonitrile (4 g, 16.4 mmol, 61.7% yield, 70.5% purity) as a yellow solid.

To a solution of 5-(aminomethyl)-N-methyl-2-methylsulfanyl-pyrimidin-4-amine (2.4 g, 12.8 mmol, 1.1 eq) and 4-oxo-2,3-dihydro-1H-quinoline-8-carbonitrile (2 g, 11.6 mmol, 1 eq) in MeOH (100 mL) was added Ti(i-PrO)₄ (9.9 g, 34.8 mmol, 10.3 mL, 3 eq) and AcOH (4.2 g, 69.7 mmol, 4.0 mL, 6 eq). The mixture was stirred at 65° C. for 60 hr. Then NaBH₃CN (4.38 g, 69.7 mmol, 6 eq) was added to the mixture, the mixture was stirred at 65° C. for 12 hr. LC-MS showed the desired compound was detected. The reaction mixture was partitioned between NaHCO₃ (100 mL) and ethyl acetate (100 mL×3). The organic phase was separated, dried over Na₂SO₄, filtered and concentrated under reduced pressure to give a residue. The residue was purified by flash silica gel chromatography (40 g Silica Flash Column, Eluent of 0-50% Ethyl acetate/Petroleum ether gradient @120 mL/min) to give 4-[[4-(methylamino)-2-methylsulfanyl-pyrimidin-5-yl] methylamino]-1,2,3,4-tetrahydroquinoline-8-carbonitrile (460 mg, 763.7 umol, 11.6% yield) as a light yellow oil.

To a solution of 4-[[4-(methylamino)-2-methylsulfanyl-pyrimidin-5-yl]methylamino]-1,2,3,4-tetrahydroquinoline-8-carbonitrile (460 mg, 1.4 mmol, 1 eq) in DCM (10 mL) was added DIEA (698.5 mg, 5.4 mmol, 941.4 uL, 4 eq) and triphosgene (320.7 mg, 1.1 mmol, 0.8 eq) at 0° C. The mixture was stirred at 25° C. for 2 hr. LC-MS showed the desired compound was detected. The reaction mixture was partitioned between NaHCO₃ (50 mL) and dichloromethane (50 mL×3). The organic phase was separated, washed with brine (20 mL×1), dried over Na₂SO₄, filtered and concentrated under reduced pressure to give 4-(1-methyl-7-methylsulfanyl-2-oxo-4H-pyrimido[4,5-d]pyrimidin-S-yl)-1,2,3,4-tetrahydroquinoline-8-carbonitrile (500 mg, crude) as a light yellow solid.

To a solution of 4-(1-methyl-7-methylsulfanyl-2-oxo-4H-pyrimido [4, 5-d]pyrimidin-S-yl)-1,2,3,4-tetrahydroquinoline-8-carbonitrile (500 mg, 1.4 mmol, 1 eq) in Boc₂O (4 mL) was added TEA (414.2 mg, 4.1 mmol, 569.7 uL, 3 eq) and DMAP (83.3 mg, 682.2 umol, 0.5 eq).

The mixture was stirred at 80° C. for 2 hr. LC-MS showed the desired compound was detected. The reaction mixture was concentrated under reduced pressure to remove solvent.

The residue was purified by flash silica gel chromatography (20 g Silica Flash Column, Eluent of 0-20% Ethyl acetate/Petroleum ether gradient @ 100 mL/min) to give tert-butyl 8-cyano-4-(1-methyl-7-methylsulfanyl-2-oxo-4H-pyrimido[4,5-d]pyrimidin-S-yl)-3,4-dihydro-2H-quinoline-1-carboxylate (600 mg, 1.3 mmol, 94.25% yield) as a yellow solid.

To a solution of tert-butyl 8-cyano-4-(1-methyl-7-methylsulfanyl-2-oxo-4H-pyrimido[4,5-d]pyrimidin-S-yl)-3,4-dihydro-2H-quinoline-1-carboxylate (600 mg, 1.3 mmol, 1 eq) in DCM (10 mL) was added mCPBA (391.6 mg, 1.9 mmol, 85% purity, 1.5 eq) at 0° C. The mixture was stirred at 25° C. for 2 hr. LC-MS showed the desired compound was detected. The reaction mixture was partitioned between Na₂SO₃ (50 mL) and dichloromethane (50 mL×3). The organic phase was separated, washed with NaHCO₃ (20 mL×3), dried over Na₂SO₄, filtered and concentrated under reduced pressure to give tert-butyl 8-cyano-4-(1-methyl-7-methylsulfonyl-2-oxo-4H-pyrimido[4,5-d]pyrimidin-S-yl)-3,4-dihydro-2H-quinoline-1-carboxylate (680 mg, crude) as a light yellow solid.

To a solution of tert-butyl 8-cyano-4-(1-methyl-7-methylsulfonyl-2-oxo-4H-pyrimido[4,5-d]pyrimidin-S-yl)-3,4-dihydro-2H-quinoline-1-carboxylate (680 mg, 341.0 umol, 1 eq) in dioxane (6 mL) was added 4-(4-methylpiperazin-1-yl)aniline (313.2 mg, 409.2 umol, 1.2 eq) and TFA (233.2 mg, 511.5 umol, 151.2 uL, 1.5 eq). The mixture was stirred at 120° C. for 12 hr. LC-MS showed the desired compound was detected. The reaction mixture was concentrated under reduced pressure to give a residue. The residue was purified by prep-HPLC (TFA condition, column: Phenomenex luna C18 100×40 mm×5 um;mobile phase: [water(TFA)-ACN];B %: 20%-50%, 8 min) to give tert-butyl 8-cyano-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (460 mg, 754.5 umol, 44.3% yield) as a light yellow solid.

To a solution of tert-butyl 8-cyano-4-[1-methyl-7-[4-(4-methylpiperazin-1-yl)anilino]-2-oxo-4H-pyrimido[4,5-d]pyrimidin-S-yl]-3,4-dihydro-2H-quinoline-1-carboxylate (230 mg, 377.2 umol, 1 eq) in DCM (5 mL) was added ZnBr₂ (1.3 g, 5.7 mmol, 283.2 uL, 15 eq). The mixture was stirred at 25° C. for 60 hr. LC-MS showed the desired compound was detected. The reaction mixture was concentrated under reduced pressure to give a residue. The residue was purified by prep-HPLC (NH₄HCO₃ condition, column: Waters Xbridge BEH C18 100×30 mm×10 um; mobile phase: [water(NH₄HCO)-ACN]; B %: 35%-55%, 8 min) to give 4-[1-methyl-7-[4-(4-methylpiperazin-1-yl)anilino]-2-oxo-4H-pyrimido[4,5-d]pyrimidin-S-yl]-1,2,3,4-tetrahydroquinoline-8-carbonitrile (160 mg, 314.0 umol, 41.6% yield) as a white solid.

To a solution of 4-[1-methyl-7-[4-(4-methylpiperazin-1-yl)anilino]-2-oxo-4H-pyrimido[4,5-d]pyrimidin-S-yl]-1,2,3,4-tetrahydroquinoline-8-carbonitrile (90 mg, 176.6 umol, 1 eq) in Tol. (3 mL) was added DIEA (114.1 mg, 883.0 umol, 153.8 uL, 5 eq) and prop-2-enoyl prop-2-enoate (44.54 mg, 353.2 umol, 1.7 uL, 2 eq). The mixture was stirred at 110° C. for 2 hr. LC-MS showed the desired compound was detected. The reaction mixture was concentrated under reduced pressure to give a residue. The residue was purified by prep-HPLC (FA condition, column: Phenomenex Luna C18 200×40 mm×10 um; mobile phase: [water(FA)-ACN]; B %: 5%-40%, 8 min) to give 4-[1-methyl-7-[4-(4-methylpiperazin-1-yl)anilino]-2-oxo-4H-pyrimido[4,5-d]pyrimidin-S-yl]-1-prop-2-enoyl-3,4-dihydro-2H-quinoline-8-carbonitrile (8.1 mg, 12.57 umol, 7.12% yield, 94.6% purity, FA) as a white solid.

### Procedure for Preparation of Compound 143

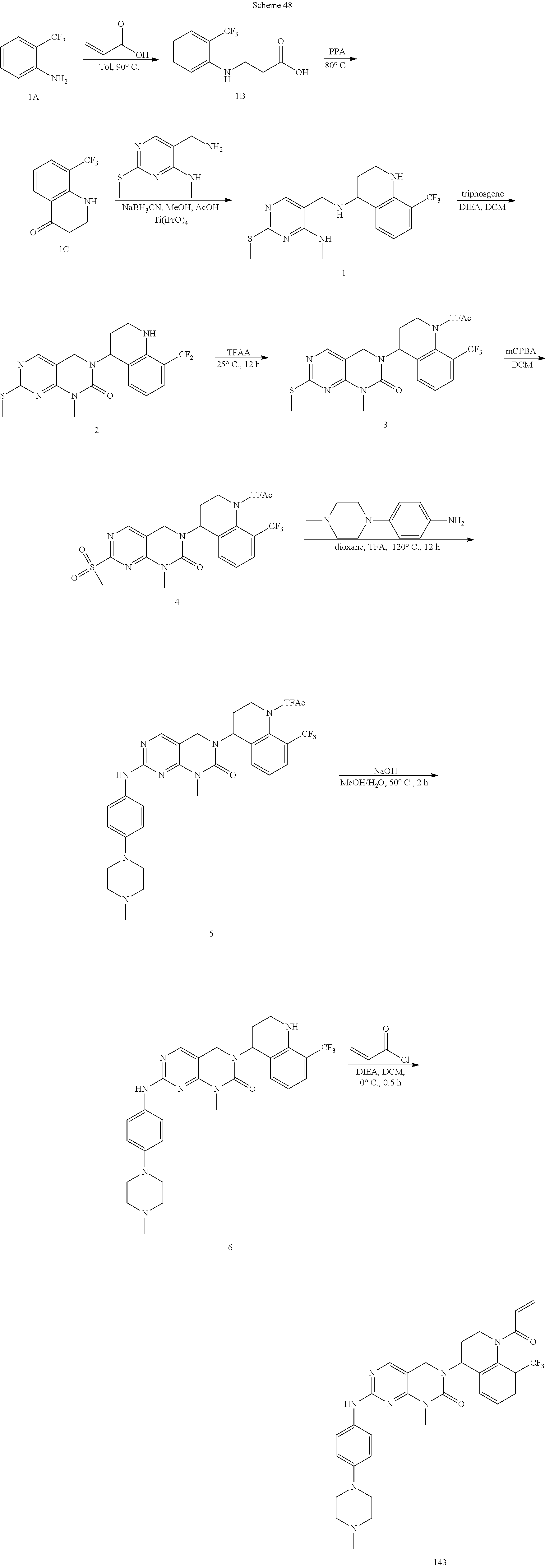

To a solution of 2-(trifluoromethyl)aniline (100 g, 620.6 mmol, 78.1 mL, 1 eq) in Tol. (500 mL) was added acrylic acid (44.7 g, 620.6 mmol, 42.6 mL, 1 eq). The mixture was stirred at 90° C. for 12 hr. LC-MS showed desired compound was detected. The reaction mixture was concentrated under reduced pressure to remove solvent. The residue was purified by flash silica gel chromatography (330 g Silica Flash Column, Eluent of 0-100% Ethyl acetate/Petroleum ether gradient @ 150 mL/min). to give compound 3-[2-(trifluoromethyl)anilino]propanoic acid (17 g, 72.9 mmol, 11.8% yield) as a yellow solid.

A mixture of 3-[2-(trifluoromethyl)anilino]propanoic acid (7 g, 30.0 mmol, 1 eq) in PPA (80 g) was stirred at 80° C. for 3 hr. LC-MS showed desired compound was detected. The reaction mixture was diluted with H₂O (200 mL). And then the pH of the reaction mixture was adjusted by addition NaOH(4M) to 7. After that, the mixture was extracted with dichloromethane (500 mL×3). The combined organic layers were dried over Na₂SO₄, filtered and concentrated under reduced pressure to give a residue. The residue was purified by flash silica gel chromatography (40 g Silica Flash Column, Eluent of 0-100% Ethyl acetate/Petroleum ether gradient @ 100 mL/min) to give compound 8-(trifluoromethyl)-2,3-dihydro-1H-quinolin-4-one (1.3 g, 4.7 mmol, 20.1% yield) as yellow solid.

To a solution of 5-(aminomethyl)-N-methyl-2-methylsulfanyl-pyrimidin-4-amine (1.1 g, 5.8 mmol, 1 eq) and 8-(trifluoromethyl)-2,3-dihydro-1 H-quinolin-4-one (1.2 g, 5.8 mmol, 1 eq) in MeOH (10 mL) was added Ti(i-PrO)₄ (4.9 g, 17.3 mmol, 5.1 mL, 3 eq) and AcOH (1.7 g, 28 mmol, 1.6 mL, 5 eq). The mixture was stirred at 65° C. for 24 h, then the mixture was cooled to 25° C., NaBH₃CN (1.8 g, 28.8 mmol, 5 eq) was added, the mixture was stirred at 65° C. for 24 hr. LC-MS showed desired compound was detected. The reaction mixture was concentrated under reduced pressure to remove MeOH. The reaction mixture was quenched by addition sat·NH₄Cl (20 mL) at 25° C. and extracted with acetate ethyl (30 mL×3). The combined organic layers were dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 80 mL/min). to give compound N-[[4-(methylamino)-2-methylsulfanyl-pyrimidin-5-yl]methyl]-8-(trifluoromethyl)-1,2,3,4-tetrahydroquinolin-4-amine (212 mg, 552.9 umol, 9.6% yield) as a yellow oil

To a solution of N-[[4-(methylamino)-2-methylsulfanyl-pyrimidin-5-yl]methyl]-8-(trifluoromethyl)-1,2,3,4-tetrahydroquinolin-4-amine (150 mg, 391.2 umol, 1 eq) in dichloromethane (4 mL) was added DIEA (252.8 mg, 2.0 mmol, 340.7 uL, 5 eq), triphosgene (169 mg, 569.5 umol, 1.5 eq) was added to the mixture at 0° C., then the mixture was stirred at 25° C. for 2 hr. LC-MS showed desired compound was detected. The reaction mixture was partitioned between NaHCO₃ (10 mL) and dichloromethane (10 mL). The water phase was separated, extracted with dichloromethane (15 mL×3), dried over Na₂SO₄, filtered and concentrated under reduced pressure to give the compound 1-methyl-7-methylsulfanyl-S-[8-(trifluoromethyl)-1,2,3,4-tetrahydroquinolin-4-yl]-4H-pyrimido[4,5-d]pyrimidin-2-one (342 mg, crude) as a yellow solid.

A solution of 1-methyl-7-methylsulfanyl-S-[8-(trifluoromethyl)-1,2,3,4-tetrahydroquinolin-4-yl]-4H-pyrimido[4,5-d]pyrimidin-2-one (200 mg, 488.5 umol, 1 eq) in TFAA (2 mL) was stirred at 25° C. for 12 hr. LC-MS showed desired compound was detected. The reaction mixture was quenched by addition sat·NaHCO₃ (25 mL) at 25° C., and extracted with ethyl acetate (20 mL×3). The combined organic layers were dried over Na₂SO₄, filtered and concentrated under reduced pressure to give compound 1-methyl-7-methylsulfanyl-S-[1-(2,2,2-trifluoroacetyl)-8-(trifluoromethyl)-3,4-dihydro-2H-quinolin-4-yl]-4H-pyrimido[4,5-d]pyrimidin-2-one (136 mg, crude) as a white solid.

To a solution of 1-methyl-7-methylsulfanyl-S-[1-(2,2,2-trifluoroacetyl)-8-(trifluoromethyl)-3,4-dihydro-2H-quinolin-4-yl]-4H-pyrimido[4,5-d]pyrimidin-2-one (136.0 mg, 269.1 umol, 1 eq) in dichloromethane (2 mL) was added mCPBA (100 mg, 492.6 umol, 85% purity, 1.8 eq) at 0° C., then the mixture was stirred at 25° C. for 2 hr. LC-MS showed desired compound was detected. The reaction mixture was quenched by addition sat·Na₂SO₃ (20 mL) at 25° C., then diluted with dichloromethane (15 mL) and extracted with dichloromethane (10 mL×3). and washed with sat. NaHCO₃ (10 mL×3), dried over Na₂SO₄, filtered and concentrated under reduced pressure to give compound 1-methyl-7-methylsulfonyl-S-[1-(2,2,2-trifluoroacetyl)-8-(trifluoromethyl)-3,4-dihydro-2H-quinolin-4-yl]-4H-pyrimido[4,5-d]pyrimidin-2-one (144 mg, crude) as a yellow solid.

To a solution of 1-methyl-7-methylsulfonyl-S-[1-(2,2,2-trifluoroacetyl)-8-(trifluoromethyl)-3,4-dihydro-2H-quinolin-4-yl]-4H-pyrimido[4,5-d]pyrimidin-2-one (124.2 mg, 231.2 umol, 1 eq) in dioxane (2 mL) was added TFA (39.5 mg, 346.8 umol, 25.7 uL, 1.5 eq) and 4-(4-methylpiperazin-1-yl)aniline (66.3 mg, 346.8 umol, 1.5 eq). The mixture was stirred at 120° C. for 12 hr. LC-MS showed desired compound was detected. The reaction was concentrated under reduce pressure. The residue was purified by prep-HPLC (TFA condition; column: Phenomenex Luna C18 100*30 mm*5 um;mobile phase: [water(TFA)-ACN];B %: 25%-50%, 8 min) to give compound 1-methyl-7-[4-(4-methylpiperazin-1-yl)anilino]-3-[1-(2,2,2-trifluoroacetyl)-8-(trifluoromethyl)-3,4-dihydro-2H-quinolin-4-yl]-4H-pyrimido[4,5-d]pyrimidin-2-one (70 mg, 107.9 umol, 46.7% yield) as a yellow oil.

To a solution of 1-methyl-7-[4-(4-methylpiperazin-1-yl)anilino]-S-[1-(2,2,2-trifluoroacetyl)-8-(trifluoromethyl)-3,4-dihydro-2H-quinolin-4-yl]-4H-pyrimido[4,5-d]pyrimidin-2-one (70 mg, 107.9 umol, 1 eq) in MeOH (1.5 mL) and H₂O (0.5 mL) was added NaOH (21.6 mg, 539.6 umol, 5 eq), then the mixture was stirred at 50° C. for 2 hr. LC-MS showed desired compound was detected. The reaction was concentrated under reduce pressure, then the residue was diluted with H₂O (10 mL) and extracted with CHCl₃/i-PrOH (5:1, 40 mL×3). The combined organic layers were dried over Na₂SO₄, filtered and concentrated under reduced pressure to give compound 1-methyl-7-[4-(4-methylpiperazin-1-yl)anilino]-S-[8-(trifluoromethyl)-1,2,3,4-tetrahydroquinolin-4-yl]-4H-pyrimido[4,5-d]pyrimidin-2-one (57 mg, crude) as a yellow oil.

To a solution of 1-methyl-7-[4-(4-methylpiperazin-1-yl)anilino]-S-[8-(trifluoromethyl)-1,2,3,4-tetrahydroquinolin-4-yl]-4H-pyrimido[4,5-d]pyrimidin-2-one (37 mg, 67.0 umol, 1 eq) in dichloromethane (0.5 mL) was added DIEA (26.0 mg, 200.9 umol, 35.0 uL, 3 eq), then prop-2-enoyl chloride (6.1 mg, 67.0 umol, 5.5 uL, 1 eq) was added to the mixture at 0° C., the mixture was stirred at 0° C. for 0.5 hr. LC-MS showed desired compound was detected. The reaction was concentrated under reduce pressure. The residue was purified by prep-HPLC (FA condition; column: C18-6 100*30 mm*5 um;mobile phase: [water(FA)-ACN];B %: 5%-45%, 8 min) to give compound 1-methyl-7-[4-(4-methylpiperazin-1-yl)anilino]-S-[1-prop-2-enoyl-8-(trifluoromethyl)-3,4-dihydro-2H-quinolin-4-yl]-4H-pyrimido[4,5-d]pyrimidin-2-one (3.9 mg, 6.1 umol, 9.1% yield, 96.0% purity) as a white solid.

### Procedure for Preparation of Compound 140

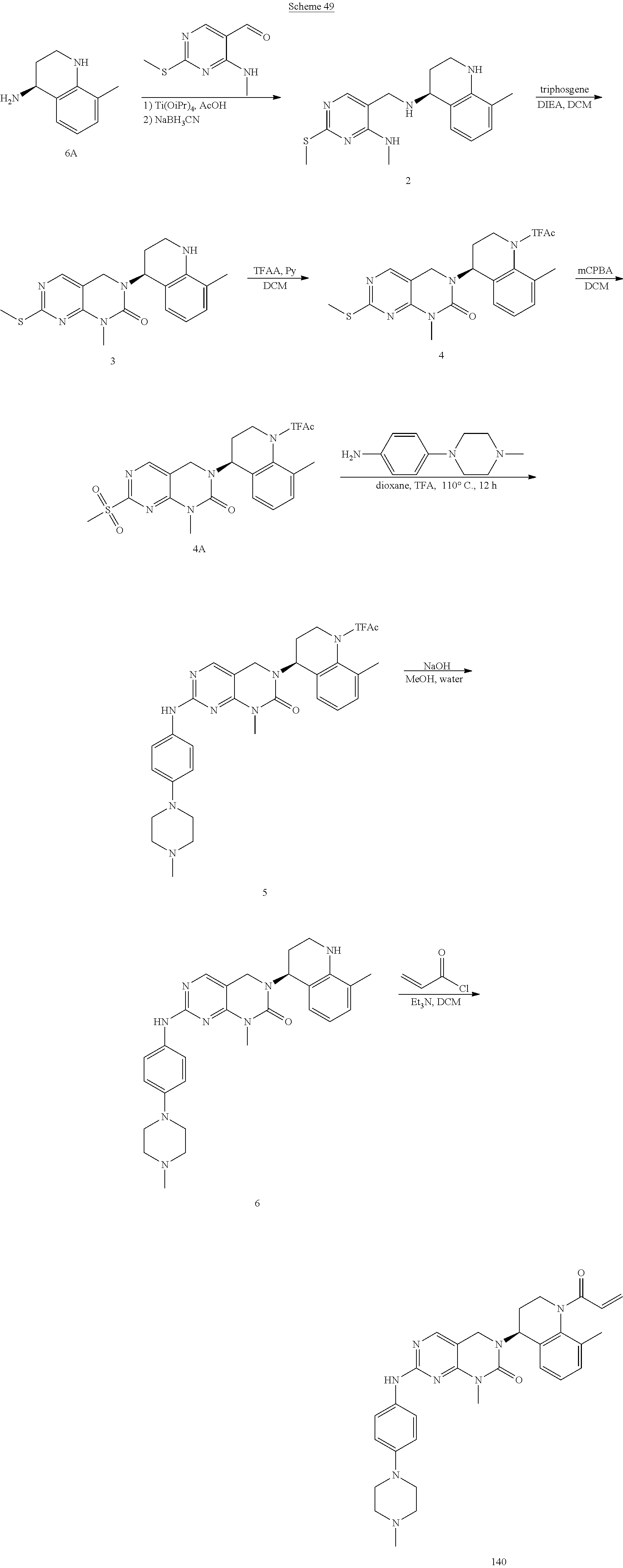

To a solution of (4S)-8-methyl-1,2,3,4-tetrahydroquinolin-4-amine (1 g, 6.2 mmol, 1 eq) 4-(methylamino)-2-methylsulfanyl-pyrimidine-5-carbaldehyde (1.1 g, 6.2 mmol, 1 eq) in MeOH (10 mL) was added Ti(i-PrO)₄ (5.3 g, 18.5 mmol, 5.5 mL, 3 eq) and AcOH (1.9 g, 30.8 mmol, 1.8 mL, 5 eg). The mixture was stirred at 60° C. for 12 hr, then the mixture was cooled 25 to 25° C., NaBH3CN (1.2 g, 18.5 mmol, 3 eq) was added, the mixture was stirred at 25° C. for 3 hr. LC-MS showed desired compound was detected. The mixture was filtered and the filtrate was quenched by Sat·NaHCO₃20 mL and then extracted by ethyl acetate (30 mL×3), the combined organic layers was dried by Na2SO₄, filtered and the filtrate was concentrated to get a residue. The residue was purified by column chromatography (SiO₂, Petroleum ether/Ethyl acetate=20/1 to 5/1) to give compound (4S)-8-methyl-N-[[4-(methylamino)-2-methylsulfanyl-pyrimidin-5-yl]methyl]-1,2,3,4-tetrahydroquinolin-4-amine (510 mg, 1.6 mmol, 25.11% yield) was obtained as a pale yellow oil.

To a solution of (4S)-8-methyl-N-[[4-(methylamino)-2-methylsulfanyl-pyrimidin-5-yl]methyl]-1,2,3,4-tetrahydroquinolin-4-amine (500 mg, 1.5 mmol, 1 eq) in DCM (10 mL) was added triphosgene (148.6 mg, 500.8 umol, 0.3 eq) and TEA (460.71 mg, 4.6 mmol, 633.7 uL, 3 eq). The mixture was stirred at 25° C. for 2 hr. LC-MS showed desired compound was detected. The mixture was quenched by Sat. NaHCO₃ 10 ml and then extracted by DCM (10 mL×3), the organic layers was dried by Na₂SO₄, filtered and the filtrate was concentrated to get a residue. The residue was purified by column chromatography (SiO₂, Petroleum ether/Ethyl acetate=3/1 to 0/1) to give compound 1-methyl-7-methylsulfanyl-S-[(4S)-8-methyl-1,2,3,4-tetrahydroquinolin-4-yl]-4H-pyrimido[4,5-d]pyrimidin-2-one (330 mg, 928.4 umol, 61.17% yield) was obtained as a colorless oil.

To a solution of 1-methyl-7-methylsulfanyl-S-[(4S)-8-methyl-1,2,3,4-tetrahydroquinolin-4-yl]-4H-pyrimido[4,5-d]pyrimidin-2-one (330 mg, 928.4 umol, 1 eq) in DCM (5 mL) was added TFAA (234.0 mg, 1.1 mmol, 154.96 uL, 1.2 eq) and Pyridine (293.7 mg, 3.7 mmol, 299.7 uL, 4 eq). The mixture was stirred at 25° C. for 12 hr. LC-MS showed desired compound was detected. The mixture was concentrated to get a residue. The residue was purified by flash silica gel chromatography (4 g Silica Flash Column, Eluent of 0-8% Ethyl acetate/Petroleum ether gradient @ 100 mL/min). to give compound 1-methyl-7-methylsulfanyl-S-[(4S)-8-methyl-1-(2,2,2-trifluoroacetyl)-3,4-dihydro-2H-quinolin-4-yl]-4H-pyrimido[4,5-d]pyrimidin-2-one (250 mg, 553.8 umol, 59.65% yield) was obtained as a white solid.

To a solution of 1-methyl-7-methylsulfanyl-S-[(4S)-8-methyl-1-(2,2,2-trifluoroacetyl)-3,4-dihydro-2H-quinolin-4-yl]-4H-pyrimido[4,5-d]pyrimidin-2-one (250 mg, 553.8 umol, 1 eq) in DCM (2 mL) was added m-CPBA (224.8 mg, 1.1 mmol, 85% purity, 2 eq). The mixture was stirred at 25° C. for 12 hr. LC-MS showed desired compound was detected. The mixture was quenched by sat. Na₂SO₃ 10 mL, then the mixture was extracted by DCM (10 mL×3), the combined organic layers was washed by sat. NaHCO₃ (10 mL×3), then dried by Na₂SO₄, filtered and the filtrate was concentrated to get compound 1-methyl-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (230 mg, crude) was obtained as a white solid.

To a solution of 1-methyl-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (230 mg, 475.7 umol, 1eq) in dioxane (2 mL) was added 4-(4-methylpiperazin-1-yl)aniline (100.1 mg, 523.3 umol, 1.1 eq) and TFA (65.09 mg, 570.88 umol, 42.27 uL, 1.2 eq). The mixture was stirred at 110° C. for 12 hr. LC-MS showed desired compound was detected. The mixture was concentrated to get a residue. The residue was purified by prep-HPLC (TFA condition;column: Phenomenex C18 75×30 mm×3 um;mobile phase: [water(TFA)-ACN];B %: 20%-50%, 8 min) to give compound 1-methyl-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (220 mg, 370.0 umol, 77.77% yield) as a brown solid.

To a solution of 1-methyl-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (220 mg, 370.0 umol, 1 eq) in Water (1 mL) was added MeOH (3 mL) and NaOH (74.0 mg, 1.9 mmol, 5 eq). The mixture was stirred at 50° C. for 8 hr. The mixture was stirred at 25° C. for 1 hr. LC-MS showed desired compound was detected. The mixture was concentrated to get a residue, and the residue was extracted by (CHCl₃/i-PrOH=3/1, 10 mL×3), the combined organic layers was dried by Na₂SO₄, then filtrated and the filtrate was concentrated to get compound 1-methyl-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (130 mg, crude) as a brown solid.

To a solution of 1-methyl-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (30 mg, 60.2 umol, 1 eq) and TEA (18.26 mg, 180.5 umol, 25.1 uL, 3 eq) in DCM (1 mL) was added prop-2-enoyl chloride (5.5 mg, 60.2 umol, 4.9 uL, 1 eq) at 0° C. The mixture was stirred at 0° C. for 1 hr. LC-MS showed desired compound was detected. The mixture was concentrated to get a residue. The residue was purified by prep-HPLC (FA condition; column: Phenomenex C18(75×30 mm×3 um);mobile phase: [water(FA)-ACN];B %: 1%-35%, 8 min) to yield 1-methyl-7-[4-(4-methylpiperazin-1-yl)anilino]-S-[(4S)-8-methyl-1-prop-2-enoyl-3,4-dihydro-2H-quinolin-4-yl]-4H-pyrimido[4,5-d]pyrimidin-2-one (5 mg, 9.05 umol, 15.04% yield, 100% purity) as a white solid

### Procedure for Preparation of Compound 141

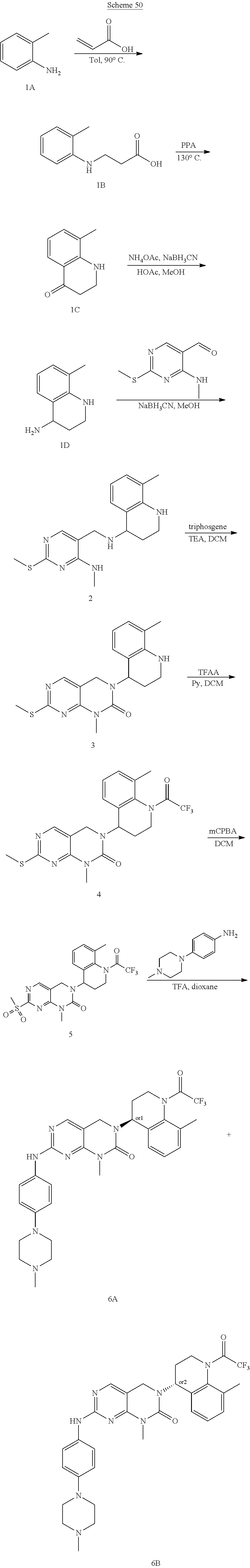

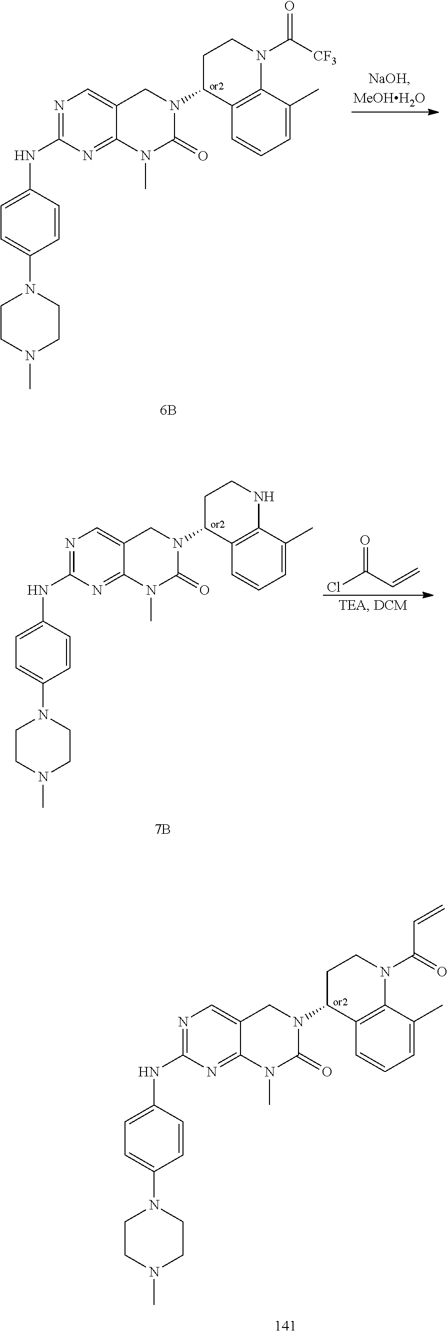

To a solution of 2-methylaniline (20 g, 93.3 mmol, 10.0 mL, 1 eq) in Tol. (200 mL) was added acrylic acid (12.2 g, 84.0 mmol, 5.8 mL, 0.9 eq). The mixture was stirred at 90° C. for 12 hr. LC-MS showed desired compound was detected. The reaction mixture was concentrated under reduced pressure to remove solvent. The residue was purified by flash silica gel chromatography (120 g Silica Flash Column, Eluent of 0-15% Ethyl acetate/Petroleum ether gradient @ 200 mL/min) to give 3-(2-methylanilino)propanoic acid (27 g, 150.7 mmol, 80.7% yield) as a yellow solid.

A mixture of 3-(2-methylanilino)propanoic acid (17 g, 94.9 mmol, 1 eq) in PPA (170 mL) was stirred at 110° C. for 8 hr. LC-MS showed desired compound was detected. The reaction mixture was quenched by addition NaOH (2 M) adjust to pH=7, and then diluted with H₂O (800 mL) and extracted with ethyl acetate (500 mL×3). The combined organic layers were washed with brine 400 mL, dried over Na₂SO₄, filtered and concentrated under reduced pressure to give a residue. The residue was purified by flash silica gel chromatography (120 g Silica Flash Column, Eluent of 0-15% Ethyl acetate/Petroleum ether gradient @ 120 mL/min) to give 8-methyl-2,3-dihydro-1H-quinolin-4-one (13.6 g, 84.4 mmol, 88.9% yield) as a brown solid.

To a solution of 8-methyl-2,3-dihydro-1 H-quinolin-4-one (7.3 g, 45.3 mmol, 1 eq) in MeOH (100 mL) was added NaBH₃CN (14.2 g, 226.4 mmol, 5 eq), NH₄OAc (62.8 g, 815.1 mmol, 18 eq) and CH₃COOH (5.4 g, 90.6 mmol, 5.2 mL, 2 eq). The mixture was stirred at 70° C. for 4 hr. LC-MS showed desired compound was detected. The reaction mixture was concentrated under reduced pressure to remove solvent. The residue was diluted with H₂O 300 mL and extracted with ethyl acetate (300 mL×3). The combined organic layers were washed with brine (250 mL×1), dried over Na₂SO₄, filtered and concentrated under reduced pressure to give a residue. The residue was purified by prep-HPLC (neutral condition) column: Biotage 800 g Agela C18; mobile phase: [water-ACN]; B %:0-30% 30 min;30% 20 min to give 8-methyl-1,2,3,4-tetrahydroquinolin-4-amine (3.1 g, 19.1 mmol, 42.2% yield) as a white solid.

To a solution of 8-methyl-1,2,3,4-tetrahydroquinolin-4-amine (2.6 g, 16.3 mmol, 1 eq) in MeOH (20 mL) was added 4-(methylamino)-2-methylsulfanyl-pyrimidine-5-carbaldehyde (3.6 g, 19.6 mmol, 1.2 eq), CH₃COOH (5.9 g, 98.0 mmol, 5.6 mL, 6 eq) and Ti(i-PrO)₄ (9.3 g, 32.7 mmol, 9.6 mL, 2 eq). The mixture was stirred at 25° C. for 12 hr. Then NaBH₃CN (3.1 g, 49.0 mmol, 3 eq) was added to the mixture. The mixture was stirred at 25° C. for 3 hr. LC-MS showed desired compound was detected. The reaction mixture was concentrated under reduced pressure to remove solvent. The residue was diluted with NaHCO₃ 300 mL and extracted with ethyl acetate (300 mL×3). The combined organic layers were washed with brine (250 mL×1), dried over Na₂SO₄, filtered and concentrated under reduced pressure to give a residue. The residue was purified by flash silica gel chromatography (20 g Silica Flash Column, Eluent of 0-33% Ethyl acetate/Petroleum ether gradient @ 120 mL/min) to give 8-methyl-N-[[4-(methylamino)-2-methylsulfanyl-pyrimidin-5-yl]methyl]-1,2,3,4-tetrahydroquinolin-4-amine (750 mg, 2.3 mmol, 13.9% yield) as a yellow solid.

To a solution of 8-methyl-N-[[4-(methylamino)-2-methylsulfanyl-pyrimidin-5-yl]methyl]-1,2,3,4-tetrahydroquinolin-4-amine (300 mg, 910.6 umol, 1 eq) in DCM (10 mL) was added TEA (460.7 mg, 4.6 mmol, 633.7 uL, 5 eq) and triphosgene (270.2 mg, 910.6 umol, 1 eq) at 0° C. The mixture was stirred at 25° C. for 2 hr. LC-MS showed desired compound was detected. The residue was diluted with NaHCO₃ 50 mL and extracted with dichloromethane (50 mL×3). The combined organic layers were washed with brine (300 mL×1), dried over Na₂SO₄, filtered and concentrated under reduced pressure to give 1-methyl-7-methylsulfanyl-S-(8-methyl-1,2,3,4-tetrahydroquinolin-4-yl)-4H-pyrimido [4,5-d]pyrimidin-2-one (170 mg, crude) as a yellow solid.

To a solution of 1-methyl-7-methylsulfanyl-S-(8-methyl-1,2,3,4-tetrahydroquinolin-4-yl)-4H-pyrimido[4,5-d]pyrimidin-2-one (120 mg, 337.6 umol, 1 eq) in DCM (2 mL) was added Py. (106.8 mg, 1.4 mmol, 109.0 uL, 4 eq) and TFAA (106.4 mg, 506.4 umol, 70.4 uL, 1.5 eq) at 0° C. The mixture was stirred at 25° C. for 12 hr. LC-MS showed desired compound was detected. The residue was diluted with H2O 50 mL and extracted with dichloromethane (40 mL×3). The combined organic layers were washed with brine (30 mL×1), dried over Na₂SO₄, filtered and concentrated under reduced pressure to give a residue. The residue was purified by prep-TLC (SiO₂, Petroleum ether: Ethyl acetate=1:1) to give 1-methyl-7-methylsulfanyl-S-[8-methyl-1-(2,2,2-trifluoroacetyl)-3,4-dihydro-2H-quinolin-4-yl]-4H-pyrimido[4,5-d] pyrimidin-2-one (110 mg, 243.6 umol, 72.2% yield) as a yellow solid.

To a solution of 1-methyl-7-methylsulfanyl-S-[8-methyl-1-(2,2,2-trifluoroacetyl)-3,4-dihydro-2H-quinolin-4-yl]-4H-pyrimido[4,5-d]pyrimidin-2-one (400 mg, 886.0 umol, 1 eq) in DCM (10 mL) was added m-CPBA (323.8 mg, 1.6 mmol, 85% purity, 1.8 eq) at 0° C. The mixture was stirred at 25° C. for 12 hr. LC-MS showed desired compound was detected. The reaction mixture was partitioned between Na₂SO₃ 30 mL and dichloromethane (30×3 mL).

The organic phase was separated, washed with NaHCO₃ (20 mL×3), dried over Na₂SO₄, filtered and concentrated under reduced pressure to give 1-methyl-7-methylsulfonyl-S-[8-methyl-1-(2,2,2-trifluoroacetyl)-3,4-dihydro-2H-quinolin-4-yl]-4H-pyrimido[4,5-d]pyrimidin-2-one (340 mg, crude) as a white solid.

To a solution of 1-methyl-7-methylsulfonyl-S-[8-methyl-1-(2,2,2-trifluoroacetyl)-3,4-dihydro-2H-quinolin-4-yl]-4H-pyrimido[4,5-d]pyrimidin-2-one (170 mg, 351.6 umol, 1 eq) in dioxane (2 mL) was added TFA (60.1 mg, 527.4 umol, 39.1 uL, 1.5 eq) and 4-(4-methylpiperazin-1-yl)aniline (80.7 mg, 422.0 umol, 1.2 eq). The mixture was stirred at 110° C. for 12 hr. LC-MS showed desired compound was detected. The reaction mixture was concentrated under reduced pressure to remove solvent. The residue was purified by prep-HPLC (TFA condition) column: Phenomenex C18 75×30 mm×3 um; mobile phase: [water(TFA)-ACN]; B %: 15%-45%, 8 min to give desired compound (300 mg, yield 71.7%, purity 96%) as a yellow solid, which was further separated by SFC (column: DAICEL CHIRALCEL OJ(250 mm×30 mm, 10 um); mobile phase: [0.1% NH₃H₂O ETOH]; B %: 45%-45%, 9 min) to give 1-methyl-7-[4-(4-methylpiperazin-1-yl)anilino]-S-[(4R)-8-methyl-1-(2,2,2-trifluoroacetyl)-3,4-dihydro-2H-quinolin-4-yl]-4H-pyrimido[4,5-d]pyrimidin-2-one (100 mg, 168.2 umol, 23.9% yield) as a yellow solid; and 1-methyl-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (95 mg, 159.7 umol, 22.7% yield) as a yellow solid.

To a solution of 1-methyl-7-[4-(4-methylpiperazin-1-yl)anilino]-S-[(4R)-8-methyl-1-(2,2,2-trifluoroacetyl)-3,4-dihydro-2H-quinolin-4-yl]-4H-pyrimido[4,5-d]pyrimidin-2-one (100 mg, 168.2 umol, 1 eq) in MeOH (3 mL)/H₂O (1 mL) was added NaOH (33.6 mg, 840.9 umol, 5 eq). The mixture was stirred at 50° C. for 12 hr. LC-MS showed desired compound was detected. The reaction mixture was diluted with H₂O 10 mL and extracted with chloroform: Isopropyl alcohol=3:1(10 mL×5). The combined organic layers were washed with brine (10 mL×3), dried over Na₂SO₄, filtered and concentrated under reduced pressure to give a residue to give 1-methyl-7-[4-(4-methylpiperazin-1-yl)anilino]-S-[(4R)-8-methyl-1,2,3,4-tetrahydroquinolin-4-yl]-4H-pyrimido[4,5-d]pyrimidin-2-one (80 mg, crude) as a yellow solid.

To a solution of 1-methyl-7-[4-(4-methylpiperazin-1-yl)anilino]-S-[(4R)-8-methyl-1,2,3,4-tetrahydroquinolin-4-yl]-4H-pyrimido[4,5-d]pyrimidin-2-one (58 mg, 116.3 umol, 1 eq) in DCM (2 mL) was added TEA (35.3 mg, 349.0 umol, 48.6 uL, 3 eq) and prop-2-enoyl chloride (10.5 mg, 116.3 umol, 9.5 uL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (FA condition) column: Phenomenex Luna C18 200×40 mm×10 um; mobile phase: [water(FA)-ACN]; B %: 10%-30%, 8 min to give 1-methyl-7-[4-(4-methylpiperazin-1-yl)anilino]-S-[(4R)-8-methyl-1-prop-2-enoyl-3,4-dihydro-2H-quinolin-4-yl]-4H-pyrimido[4,5-d]pyrimidin-2-one (17.2 mg, 30.8 umol, 26.5% yield, 98.7% purity) as a white solid.

### Procedure for Preparation of Compound 147

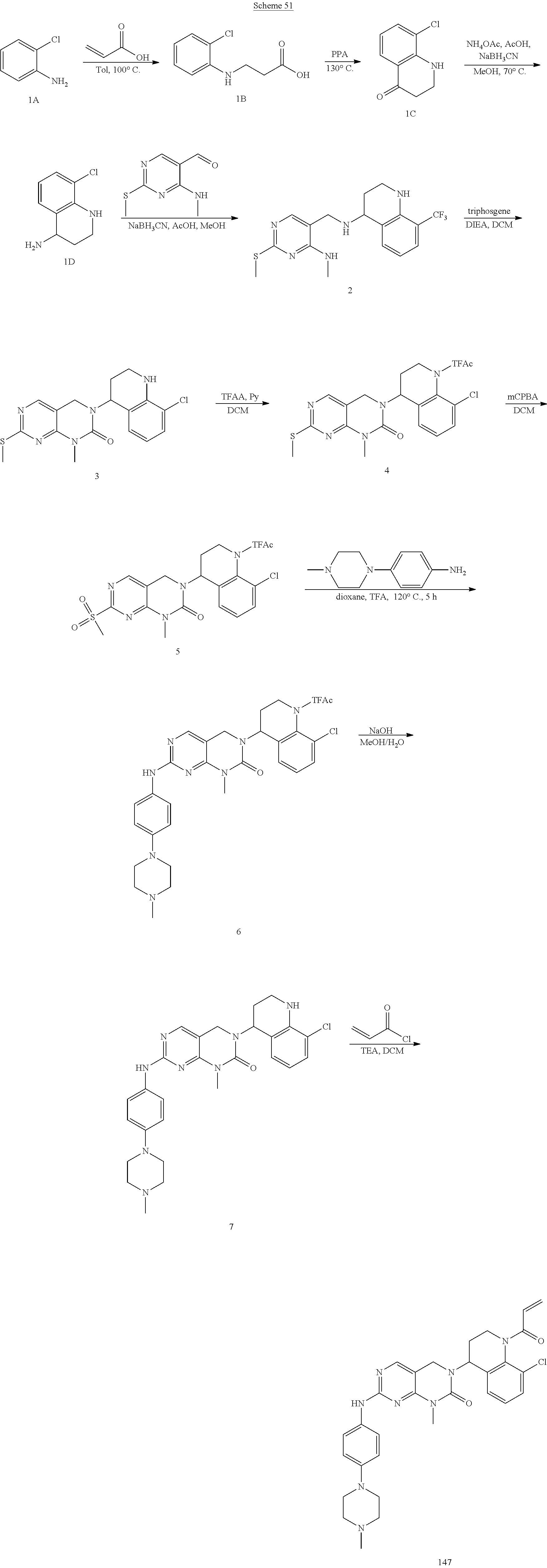

To a solution of 2-chloroaniline (10 g, 78.3 mmol, 8.2 mL, 1 eq) in Tol. (500 mL) was added acrylic acid (8.4 g, 117.5 mmol, 8.0 mL, 1.5 eq). The mixture was stirred at 100° C. for 12 hr. LC-MS showed desired mass was detected. The reaction mixture was concentrated under reduced pressure to remove solvent. The crude product was triturated with Ethyl acetate at 25° C. for 12 hr and filtered to give 3-(2-chloroanilino)propanoic acid (47.9 g, 233.5 mmol, 59.6% yield, 97.2% purity) as a yellow solid.

A mixture of 3-(2-chloroanilino)propanoic acid (15.9 g, 80.0 mmol, 1 eq) and PPA (159.7 mL) was stirred at 130° C. for 2 hr. LC-MS showed desired mass was detected. The reaction mixture was added to 15% NaOH 1000 mL at 0° C., and then diluted with H₂O 1500 mL and extracted with Ethyl acetate 3000 mL (1000 mL×3), dried over Na₂SO₄, filtered and concentrated under reduced pressure to give a residue. The residue was purified by flash silica gel chromatography (40 g Silica Flash Column, Eluent of 0-20% Ethyl acetate/Petroleum ether gradient @ 100 mL/min) to give 8-chloro-2,3-dihydro-1 H-quinolin-4-one (21.2 g, 109.5 mmol, 45.6% yield, 93.8% purity) as a yellow solid.

To a solution of 8-chloro-2,3-dihydro-1 H-quinolin-4-one (5 g, 27.5 mmol, 1 eq) in MeOH (50 mL) was added NH₄OAc (42.4 g, 550.6 mmol, 20 eq), NaBH₃CN (8.6 g, 137.6 mmol, 5 eq) and AcOH (3.3 g, 55.0 mmol, 3.1 mL, 2 eq). The mixture was stirred at 70° C. for 12 hr. LC-MS showed desired mass was detected. The reaction mixture was concentrated under reduced pressure to remove solvent. The residue was diluted with H₂O 60 mL and extracted with Ethyl acetate 90 mL (30 mL×3), dried over Na₂SO₄, filtered and concentrated under reduced pressure to give a residue. The residue was purified by prep-HPLC (neutral condition, column: Biotage 800 g Agela C18;mobile phase: [water-MeCN];B %:0-25% 30 min;25% 15 min) to give 8-chloro-1,2,3,4-tetrahydroquinolin-4-amine (4.7 g, 25.7 mmol, 93.5% yield, 98.8% purity) as a colorless oil.

To a solution of 8-chloro-1,2,3,4-tetrahydroquinolin-4-amine (2 g, 10.9 mmol, 1 eq) in MeOH (20 mL) was added 4-(methylamino)-2-methylsulfanyl-4,5-dihydropyrimidine-5-carbaldehyde (2.0 g, 10.9 mmol, 1 eq) and AcOH (1.9 g, 32.8 mmol, 1.8 mL, 3 eq) to adjust pH to 5-6. The mixture was stirred at 65° C. for 12 hr. NaBH₃CN (2.0 g, 32.8 mmol, 3 eq) was then added, and stirred at 65° C. for 2 hr. LC-MS showed desired mass was detected. The reaction mixture was concentrated under reduced pressure to remove solvent. The reaction mixture was diluted with H₂O 40 mL and extracted with Ethyl acetate 60 mL (20 mL×3), dried over Na₂SO₄, filtered and concentrated under reduced pressure to give a residue. The residue was purified by flash silica gel chromatography (20 g Silica Flash Column, Eluent of 0-30% Ethyl acetate/Petroleum ether gradient @ 100 mL/min) to give 8-chloro-N-[[4-(methylamino)-2-methylsulfanyl-pyrimidin-5-yl]methyl]-1,2,3,4-tetrahydroquinolin-4-amine (1.2 g, 3.1 mmol, 29.0% yield, 89% purity) as orange oil.

To a solution of 8-chloro-N-[[4-(methylamino)-2-methylsulfanyl-pyrimidin-5-yl]methyl]-1,2,3,4-tetrahydroquinolin-4-amine (1.2 g, 3.5 mmol, 1 eq) in DCM (15 mL) was added DIEA (1.3 g, 10.7 mmol, 1.8 mL, 3 eq) and bis(trichloromethyl) carbonate (954.1 mg, 3.2 mmol, 0.9 eq) at 0° C. The mixture was stirred at 0° C. for 2 hr. LC-MS showed desired mass was detected. The residue was diluted with NaHCO₃ 15 mL and extracted with Dichloromethane (15 mL×3). The combined organic layers were washed with brine (13 mL×3), dried over Na₂SO₄, filtered and concentrated under reduced pressure to give a residue. The residue was purified by flash silica gel chromatography (20 g Silica Flash Column, Eluent of 0-50% Ethyl acetate/Petroleum ether gradient @ 100 mL/min) to give 3-(8-chloro-1,2,3,4-tetrahydroquinolin-4-yl)-1-methyl-7-methylsulfanyl-4H-pyrimido[4,5-d]pyrimidin-2-one (480 mg, 1.2 mmol, 35.0% yield, 98% purity) as a white solid.

To a solution of 3-(8-chloro-1,2,3,4-tetrahydroquinolin-4-yl)-1-methyl-7-methylsulfanyl-4H-pyrimido[4,5-d]pyrimidin-2-one (480 mg, 1.2 mmol, 1 eq) in DCM (15 mL) was added pyridine (404.0 mg, 5.1 mmol, 412.2 uL, 4 eq) at 0° C. and then TFAA (402.3 mg, 1.92 mmol, 266.4 uL, 1.5 eq) was added. The mixture was stirred at 20° C. for 2 hr. LC-MS showed desired mass was detected. The residue was diluted with H2O 30 mL and extracted with Dichloromethane 90 mL (30 mL×3), dried over Na₂SO₄, filtered and concentrated under reduced pressure to give 3-[8-chloro-1-(2,2,2-trifluoroacetyl)-3,4-dihydro-2H-quinolin-4-yl]-1-methyl-7-methylsulfanyl-4H-pyrimido[4,5-d]pyrimidin-2-one (731 mg, crude) as colorless oil.

To a solution of 3-[8-chloro-1-(2,2,2-trifluoroacetyl)-3,4-dihydro-2H-quinolin-4-yl]-1-methyl-7-methylsulfanyl-4H-pyrimido[4,5-d]pyrimidin-2-one (731 mg, 1.5 mmol, 1 eq) in DCM (15 mL) was added m-CPBA (471.7 mg, 2.3 mmol, 85% purity, 1.5 eq) at 0° C. The resulting mixture was stirred at 0° C. for 3 hr. LC-MS showed 3-[8-chloro-1-(2,2,2-trifluoroacetyl)-3,4-dihydro-2H-quinolin-4-yl]-1-methyl-7-methylsulfanyl-4H-pyrimido[4,5-d]pyrimidin-2-one remained and desired compound was detected. m-CPBA (314.5 mg, 1.5 mmol, 85% purity, 1 eq) was added, and the mixture was stirred at 25° C. for 1 hr. LC-MS showed desired mass was detected. The reaction mixture was quenched by addition Na₂SO₃ 40 mL, extracted with Dichloromethane 60 mL (20 mL×3), and dried over Na₂SO₄, filtered and concentrated under reduced pressure to give 3-[8-chloro-1-(2,2,2-trifluoroacetyl)-3,4-dihydro-2H-quinolin-4-yl]-1-methyl-7-methylsulfonyl-4H-pyrimido[4,5-d]pyrimidin-2-one (673.8 mg, crude) as yellow solid.

To a solution of 3-[8-chloro-1-(2,2,2-trifluoroacetyl)-3,4-dihydro-2H-quinolin-4-yl]-1-methyl-7-methylsulfonyl-4H-pyrimido[4,5-d]pyrimidin-2-one (673.8 mg, 1.3 mmol, 1 eq) in dioxane (5 mL) was added 4-(4-methylpiperazin-1-yl)aniline (383.6 mg, 2.0 mmol, 1.5 eq) and TFA (228.7 mg, 2.0 mmol, 148.5 uL, 1.5 eq). The mixture was stirred at 120° C. for 5 hr. LC-MS showed desired mass was detected. The reaction mixture was concentrated under reduced pressure to remove solvent. The residue was purified by prep-HPLC (FA condition, column: Phenomenex C18(75×30 mm×3 um);mobile phase: [water(FA)-ACN];B %: 15%-45%, 8 min) to give 3-[8-chloro-1-(2,2,2-trifluoroacetyl)-3,4-dihydro-2H-quinolin-4-yl]-1-methyl-7-[4-(4-methylpiperazin-1-yl)anilino]-4H-pyrimido[4,5-d]pyrimidin-2-one (332.5 mg, 529.8 umol, 39.6% yield, 98% purity) as a yellow solid.

To a solution of 3-[8-chloro-1-(2,2,2-trifluoroacetyl)-3,4-dihydro-2H-quinolin-4-yl]-1-methyl-7-[4-(4-methylpiperazin-1-yl)anilino]-4H-pyrimido[4,5-d]pyrimidin-2-one (244.7 mg, 397.8 umol, 1 eq) in MeOH (2.1 mL) and H₂O (0.7 mL) was added NaOH (79.5 mg, 1.9 mmol, 5 eq). The mixture was stirred at 50° C. for 1 hr. LC-MS showed desired mass was detected. The reaction mixture was concentrated under reduced pressure to remove solvent. The residue was diluted with H₂O 20 mL and extracted with Chloroform: Isopropyl alcohol 10:1 30 mL (10 mL×3), the combined organic phase was dried over Na₂SO₄, filtered and concentrated under reduced pressure to give 3-(8-chloro-1,2,3,4-tetrahydroquinolin-4-yl)-1-methyl-7-[4-(4-methylpiperazin-1-yl)anilino]-4H-pyrimido[4,5-d]pyrimidin-2-one (165.8 mg, crude) as a yellow solid.

To a solution of 3-(8-chloro-1,2,3,4-tetrahydroquinolin-4-yl)-1-methyl-7-[4-(4-methylpiperazin-1-yl)anilino]-4H-pyrimido[4,5-d]pyrimidin-2-one (85.8 mg, 165.3 umol, 1 eq) in DCM (1.5 mL) was added TEA (50.1 mg, 495.9 umol, 69.0 uL, 3 eq) to adjust pH to 8-9. And then prop-2-enoyl chloride (14.9 mg, 165.3 umol, 13.4 uL, 1 eq) in DCM (0.2 mL) was added dropwise at 0° C. The mixture was stirred at 0° C. for 1 hr. LC-MS showed the reactant remained mostly. Prop-2-enoyl chloride (14.9 mg, 165.3 umol, 13.4 uL, 1 eq) in DCM (0.2 mL) was added dropwise at 0° C. The mixture was stirred at 0° C. for 1 hr. LC-MS showed desired mass was detected. The reaction mixture was concentrated under reduced pressure to remove solvent. The residue was purified by prep-HPLC (TFA condition: column: Phenomenex luna C18 100×40 mm×5 um;mobile phase: [water(TFA)-ACN];B %: 1%-35%, 8 min) to give 3-(8-chloro-1-prop-2-enoyl-3,4-dihydro-2H-quinolin-4-yl)-1-methyl-7-[4-(4-methylpiperazin-1-yl)anilino]-4H-pyrimido[4,5-d]pyrimidin-2-one (6.0 mg, 9.9 umol, 6.0% yield, 94% purity) as a yellow solid.

### Procedure for Preparation of Compound 146

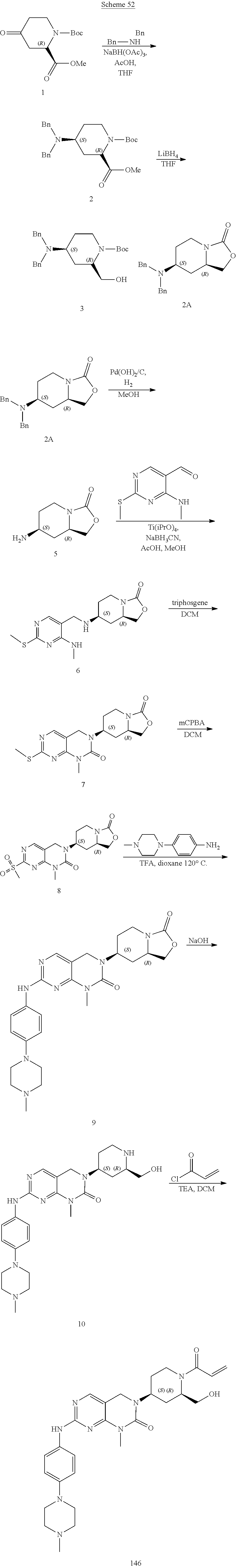

To a solution of N-benzyl-1-phenyl-methanamine (3.8 g, 19.4 mmol, 3.7 mL, 1 eq) in THE (70 mL) was added O1-tert-butyl 02-methyl (2R)-4-oxopiperidine-1,2-dicarboxylate (5 g, 19.4 mmol, 1 eq) and AcOH (5.2 g, 87.4 mmol, 5.00 mL, 4.5 eq). The mixture was stirred at 20° C. for 12 hr. NaBH(OAc)₃ (12.3 g, 58.3 mmol, 3 eq) was added. The mixture was stirred at 20° C. for 12 hr. LC-MS showed desired mass was detected. The reaction mixture was partitioned between H₂O 50 mL and Ethyl acetate (50×3 mL). The organic phase was separated, washed with brine (50 mL×3), dried over Na₂SO₄, filtered and concentrated under reduced pressure to give a residue. The residue was purified by flash silica gel chromatography (20 g Silica Flash Column, Eluent of 0-20% Ethyl acetate/Petroleum ether gradient @ 100 mL/min) to give 01-tert-butyl 02-methyl (2R,4S)-4-(dibenzylamino)piperidine-1,2-dicarboxylate (2.8 g, 4.4 mmol, 23.0% yield, 70% purity) as colorless oil.

To a solution of 01-tert-butyl 02-methyl (2R,4S)-4-(dibenzylamino)piperidine-1,2-dicarboxylate (2.7 g, 6.2 mmol, 1 eq) in THE (20 mL) was added LiBH₄ (273.1 mg, 12.5 mmol, 2 eq) at 0° C. under N₂. The mixture was stirred at 20° C. for 14 hr. LC-MS showed desired mass was detected. The reaction mixture was quenched between H₂O 50 mL and HCl (60 mL, 1 M) and extracted with Ethyl acetate (50×3 mL). The organic phase was separated, washed with brine (50 mL×3), dried over Na₂SO₄, filtered and concentrated under reduced pressure to give a residue. The residue was purified by flash silica gel chromatography (12 g Silica Flash Column, Eluent of 0-20% Ethyl acetate/Petroleum ether gradient @ 100 mL/min). to give (7S,8aR)-7-(dibenzylamino)-1,5,6,7,8,8a-hexahydrooxazolo[3,4-a]pyridin-S-one (1.1 g, 2.94 mmol, 46.93% yield, 90% purity) as colorless oil and tert-butyl (2R,4S)-4-(dibenzylamino)-2-(hydroxymethyl)piperidine-1-carboxylate (650 mg, 1.3 mmol, 21.2% yield, 84% purity) as colorless oil.

To a solution of (7S,8aR)-7-(dibenzylamino)-1,5,6,7,8,8a-hexahydrooxazolo[3,4-a]pyridin-S-one (1 g, 2.9 mmol, 1 eq) in MeOH (10 mL) was added Pd(OH)₂ (208.7 mg, 297.2 umol, 20% purity, 0.1 eq) under N₂ atmosphere. The suspension was degassed and purged with H₂ for 3 times. The mixture was stirred under H₂ (15 Psi) at 20° C. for 12 hr. TLC indicated (7S,8aR)-7-(dibenzylamino)-1,5,6,7,8,8a-hexahydrooxazolo[3,4-a]pyridin-S-one was consumed completely and one new spot formed. The reaction mixture was filtered and concentrated under reduced pressure to give (7S,8aR)-7-amino-1,5,6,7,8,8a-hexahydrooxazolo[3,4-a]pyridin-S-one (330 mg, crude) as a white solid.

To a solution of (7S,8aR)-7-amino-1,5,6,7,8,8a-hexahydrooxazolo[3,4-a]pyridin-S-one (330 mg, 2.1 mmol, 1 eq) in MeOH (1 mL) was added 4-(methylamino)-2-methylsulfanyl-pyrimidine-5-carbaldehyde (387.1 mg, 2.1 mmol, 1 eq), AcOH (570.9 mg, 9.5 mmol, 543.7 uL, 4.5 eq) and Ti(i-PrO)₄ (1.8 g, 6.3 mmol, 1.8 mL, 3 eq). The mixture was stirred at 70° C. for 12 hr. NaBH₃CN (398.3 mg, 6.3 mmol, 3 eq) was added. The mixture was stirred at 70° C. for 2 hr. LC-MS showed desired mass was detected. The residue was diluted with NaHCO₃ 15 mL and extracted with Dichloromethane (15 mL×3). The combined organic layers were washed with brine (13 mL×3), dried over Na₂SO₄, filtered and concentrated under reduced pressure to give (7S,8aR)-7-[[4-(methylamino)-2-methylsulfanyl-pyrimidin-5-yl]methylamino]-1,5,6,7,8,8a-hexahydrooxazolo[3,4-a]pyridin-S-one (750 mg, crude) as colorless oil.

To a solution of (7S,8aR)-7-[[4-(methylamino)-2-methylsulfanyl-pyrimidin-5-yl]methylamino]-1,5,6,7,8,8a-hexahydrooxazolo[3,4-a]pyridin-S-one (750 mg, 2.3 mmol, 1 eq) in DCM (10 mL) was added DIEA (899.1 mg, 6.9 mmol, 1.21 mL, 3 eq) and bis(trichloromethyl) carbonate (412.9 mg, 1.3 mmol, 0.6 eq) at 0° C. The mixture was stirred at 20° C. for 2 hr. LC-MS showed desired mass was detected. The residue was diluted with NaHCO₃ 30 mL and extracted with Dichloromethane (30 mL×3). The combined organic layers were washed with brine (30 mL×3), dried over Na₂SO₄, filtered and concentrated under reduced pressure to give a residue. The residue was purified by flash silica gel chromatography (12 g Silica Flash Column, Eluent of 0-100% Ethyl acetate/Petroleum ether gradient @ 100 mL/min) to give (7S,8aR)-7-(1-methyl-7-methylsulfanyl-2-oxo-4H-pyrimido[4,5-d]pyrimidin-S-yl)-1,5,6,7,8,8a-hexahydrooxazolo[3,4-a]pyridin-S-one (411 mg, 941.0 umol, 40.5% yield, 80% purity) as a white solid.

To a solution of (7S,8aR)-7-(1-methyl-7-methylsulfanyl-2-oxo-4H-pyrimido[4,5-d]pyrimidin-S-yl)-1,5,6,7,8,8a-hexahydrooxazolo[3,4-a]pyridin-S-one (390 mg, 1.1 mmol, 1 eq) in DCM (10 mL) was added m-CPBA (339.9 mg, 1.6 mmol, 85% purity, 1.5 eq) at 0° C.

The mixture was stirred at 0° C. for 2 hr. The mixture was stirred at 0° C. for 12 hr. LC-MS showed desired mass was detected. The reaction mixture was diluted with Na₂SO₃ 10 mL and extracted with Dichloromethane 6 mL (2 mL×3), dried over Na₂SO₄, filtered and concentrated under reduced pressure to give (7S,8aR)-7-(1-methyl-7-methylsulfonyl-2-oxo-4H-pyrimido[4,5-d]pyrimidin-S-yl)-1,5,6,7,8,8a-hexahydrooxazolo[3,4-a]pyridin-S-one (417 mg, crude) as a white solid.

To a solution of (7S,8aR)-7-(1-methyl-7-methylsulfonyl-2-oxo-4H-pyrimido[4,5-d]pyrimidin-S-yl)-1,5,6,7,8,8a-hexahydrooxazolo[3,4-a]pyridin-S-one (417 mg, 1.09 mmol, 1 eq) in dioxane (5 mL) was added TFA (186.4 mg, 1.6 mmol, 121.0 uL, 1.5 eq) and 4-(4-methylpiperazin-1-yl)aniline (312.7 mg, 1.6 mmol, 1.5 eq). The mixture was stirred at 120° C. for 12 hr. LC-MS showed desired compound was detected. The reaction mixture was concentrated under reduced pressure to remove solvent. The residue was purified by prep-HPLC (FA condition, column: Phenomenex C18(75×30 mm×3 um);mobile phase: [water(FA)-ACN]; 8 min) to give (7S,8aR)-7-[1-methyl-7-[4-(4-methylpiperazin-1-yl)anilino]-2-oxo-4H-pyrimido[4,5-d]pyrimidin-S-yl]-1,5,6,7,8,8a-hexahydrooxazolo[3,4-a]pyridin-S-one (138 mg, 280.1 umol, 25.7% yield) as a brown solid.

To a solution of (7S,8aR)-7-[1-methyl-7-[4-(4-methylpiperazin-1-yl)anilino]-2-oxo-4H-pyrimido[4,5-d]pyrimidin-S-yl]-1,5,6,7,8,8a-hexahydrooxazolo[3,4-a]pyridin-S-one (138 mg, 280.1 umol, 1 eq) in EtOH (2 mL) was added NaOH (2 M, 700.4 uL, 5 eq). The mixture was stirred at 50° C. for 2 hr. LC-MS showed desired compound was detected. The mixture was stirred at 50° C. for 12 hr. LC-MS showed desired mass was detected. The residue was diluted with H₂O 15 mL and extracted with Dichloromethane: Methanol=10:1 (15 mL×3). The combined organic layers were washed with brine (13 mL×3), dried over Na₂SO₄, filtered and concentrated under reduced pressure to give 3-[(2R,4S)-2-(hydroxymethyl)-4-piperidyl]-1-methyl-7-[4-(4-methylpiperazin-1-yl)anilino]-4H-pyrimido[4,5-d]pyrimidin-2-one (70 mg, crude) as a yellow solid.

To a solution of 3-[(2R,4S)-2-(hydroxymethyl)-4-piperidyl]-1-methyl-7-[4-(4-methylpiperazin-1-yl)anilino]-4H-pyrimido[4,5-d]pyrimidin-2-one (46 mg, 98.5 umol, 1 eq) in DCM (1 mL) was added TEA (29.9 mg, 295.7 umol, 41.1 uL, 3 eq) and prop-2-enoyl chloride (8.9 mg, 98.5 umol, 8.0 uL, 1 eq) at 0° C. The mixture was stirred at 0° C. for 1 hr. The reaction mixture was concentrated under reduced pressure to remove solvent. The residue was purified by prep-HPLC (TFA condition, column: Phenomenex luna C18 100×40 mm×5 um;mobile phase: [water(TFA)-ACN];B %: 1%-15%, 8 min) to give 3-[(2R,4S)-2-(hydroxymethyl)-1-prop-2-enoyl-4-piperidyl]-1-methyl-7-[4-(4-methylpiperazin-1-yl)anilino]-4H-pyrimido[4,5-d]pyrimidin-2-one (9.1 mg, 17.0 umol, 17.2% yield, 96.6% purity) as a yellowish green solid.

### Procedure for Preparation of Compound 152

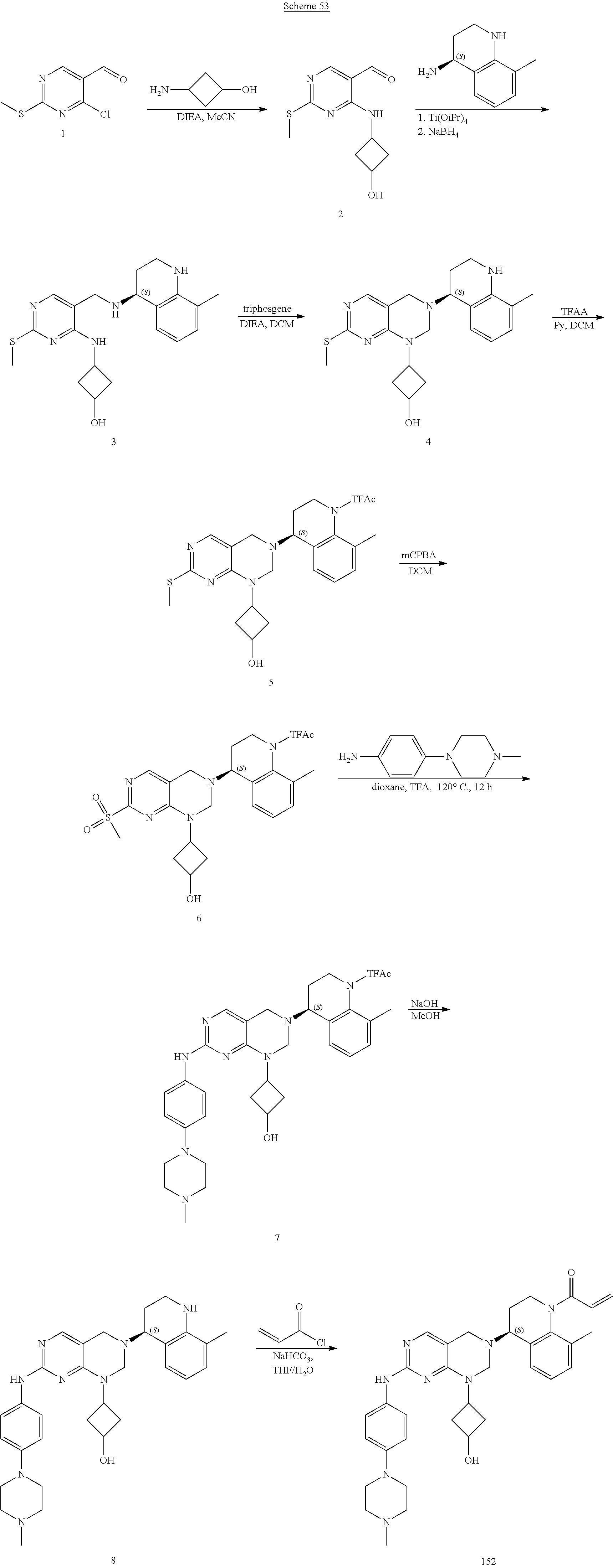

To a solution of 4-chloro-2-methylsulfanyl-pyrimidine-5-carbaldehyde (1 g, 5.3 mmol, 1 eq) in MeCN (20 mL) was added DIEA (2.1 g, 15.9 mmol, 2.8 mL, 3 eq) and 3-aminocyclobutanol (461.8 mg, 5.3 mmol, 1 eq). The mixture was stirred at 25° C. for 2 hr. LC-MS showed desired compound was detected. The reaction mixture was concentrated under reduced pressure to remove solvent. The residue was purified by flash silica gel chromatography (12 g Silica Flash Column, Eluent of 0˜30% Ethyl acetate/Petroleum ether gradient @ 100 mL/min) to give 4-[(3-hydroxycyclobutyl)amino]-2-methylsulfanyl-pyrimidine-5-carbaldehyde (664.2 mg, 2.8 mmol, 52.4% yield) as a white solid.

To a solution of 4-[(3-hydroxycyclobutyl)amino]-2-methylsulfanyl-pyrimidine-5-20 carbaldehyde (1.5 g, 6.3 mmol, 1 eq) in MeOH (15 mL) was added Ti(i-PrO)₄ (5.3 g, 18.8 mmol, 5.6 mL, 3 eq) and (4S)-8-methyl-1,2,3,4-tetrahydroquinolin-4-amine (1.0 g, 6.3 mmol, 1 eq). The mixture was stirred at 65° C. for 12 hr. Then NaBH₄ (1.2 g, 31.3 mmol, 5 eq) was added to the mixture at 0° C. The mixture was stirred at 25° C. for 2 hr. LC-MS showed desired compound was detected. The reaction mixture was concentrated under reduced pressure to remove solvent. The residue was diluted with NH₄Cl 300 mL and extracted with ethyl acetate (300 mL×3). The combined organic layers were washed with brine (250 mL×1), dried over Na₂SO₄, filtered and concentrated under reduced pressure to give a residue. The residue was purified by flash silica gel chromatography (20 g Silica Flash Column, Eluent of 0-42% Ethyl acetate/Petroleum ether gradient @ 120 mL/min) to give 3-[[2-methylsulfanyl-5-[[[(4S)-8-methyl-1,2,3,4-tetrahydroquinolin-4-yl]amino]methyl]pyrimidin-4-yl]amino]cyclobutanol (1.7 g, 4.4 mmol, 70.4% yield) as a yellow solid.

To a solution of 3-[[2-methylsulfanyl-5-[[[(4S)-8-methyl-1,2,3,4-tetrahydroquinolin-4-yl]amino]methyl]pyrimidin-4-yl]amino]cyclobutanol (2.1 g, 5.4 mmol, 1 eq) in DCM (20 mL) was added DIEA (2.1 g, 16.3 mmol, 2.8 mL, 3 eq) and triphosgene (533.4 mg, 1.8 mmol, 0.33 eq) at 0° C. The mixture was stirred at 25° C. for 12 hr. LC-MS showed desired compound was detected. The residue was diluted with NaHCO₃ 50 mL and extracted with dichloromethane (50 mL×3). The combined organic layers were washed with brine (300 mL×1), dried over Na₂SO₄, filtered and concentrated under reduced pressure to give 1-(3-hydroxycyclobutyl)-7-methylsulfanyl-S-[(4S)-8-methyl-1,2,3,4-tetrahydroquinolin-4-yl]-4H-pyrimido[4,5-d]pyrimidin-2-one (1.6 g, crude) as a yellow solid.

To a solution of 1-(3-hydroxycyclobutyl)-7-methylsulfanyl-S-[(4S)-8-methyl-1,2,3,4-tetrahydroquinolin-4-yl]-4H-pyrimido[4,5-d]pyrimidin-2-one (1.6 g, 3.9 mmol, 1 eq) in DCM (20 mL) was added Py. (1.2 g, 15.6 mmol, 1.3 mL, 4.0 eq) and TFAA (1.2 g, 5.8 mmol, 811.2 uL, 1.5 eq) at 0° C. The mixture was stirred at 25° C. for 12 hr. LC-MS showed desired compound was detected. The residue was diluted with H2 O 50 mL and extracted with dichloromethane (40 mL×3). The combined organic layers were washed with brine (30 mL×1), dried over Na₂SO₄, filtered and concentrated under reduced pressure to give a residue.

The residue was purified by flash silica gel chromatography (40 g Silica Flash Column, Eluent of 0-12% Ethyl acetate/Petroleum ether gradient @ 120 mL/min) to give 1-(3-hydroxycyclobutyl)-7-methylsulfanyl-S-[(4S)-8-methyl-1-(2,2,2-trifluoroacetyl)-3,4-dihydro-2H-quinolin-4-yl]-4H-pyrimido[4,5-d] pyrimidin-2-one (1.4 g, 2.7 mmol, 68.4% yield) as a yellow solid.

To a solution of 1-(3-hydroxycyclobutyl)-7-methylsulfanyl-S-[(4S)-8-methyl-1-(2,2,2-trifluoroacetyl)-3,4-dihydro-2H-quinolin-4-yl]-4H-pyrimido[4,5-d]pyrimidin-2-one (220 mg, 433.5 umol, 1 eq) in DCM (2 mL) was added m-CPBA (132.0 mg, 650.2 umol, 85% purity, 1.5 eq) at 0° C. The mixture was stirred at 25° C. for 2 hr. LC-MS showed desired compound was detected. The reaction mixture was partitioned between Na₂SO₃ 30 mL and dichloromethane (30×3 mL). The organic phase was separated, washed with NaHCO₃ (20 mL×3), dried over Na₂SO₄, filtered and concentrated under reduced pressure to give 1-(3-hydroxycyclobutyl)-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (220 mg, crude) as a yellow solid.

To a solution of 1-(3-hydroxycyclobutyl)-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (170 mg, 315.1 umol, 1 eq) in dioxane (2 mL) was added TFA (53.9 mg, 472.6 umol, 35.0 uL, 1.5 eq) and 4-(4-methylpiperazin-1-yl)aniline (72.3 mg, 378.1 umol, 1.2 eq). The mixture was stirred at 120° C. for 12 hr. LC-MS showed desired compound was detected. The reaction mixture was concentrated under reduced pressure to remove solvent. The residue was purified by prep-HPLC (TFA condition) column: Phenomenex C18 75×30 mm×3 um; mobile phase: [water(TFA)-ACN]; B %: 10%-40%, 8 min to give 1-(3-hydroxycyclobutyl)-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (530 mg, 814.5 umol, 73.2% yield) as a yellow solid.

To a solution of 1-(3-hydroxycyclobutyl)-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (300 mg, 461.1 umol, 1 eq) in MeOH (2 mL)/H₂O (2 mL) was added NaOH (92.2 mg, 2.3 mmol, 5 eq). The mixture was stirred at 50° C. for 1 hr. LC-MS showed desired compound was detected. The reaction mixture was diluted with H₂O 10 mL and extracted with chloroform: Isopropyl alcohol=3:1(10 mL×5). The combined organic layers were washed with brine (10 mL×3), dried over Na₂SO₄, filtered and concentrated under reduced pressure to give a residue to give 1-(3-hydroxycyclobutyl)-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (180 mg, crude) as a yellow oil.

To a solution of 1-(3-hydroxycyclobutyl)-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (100 mg, 180.3 umol, 1 eq) and NaHCO₃ (45.4 mg, 540.8 umol, 21.0 uL, 3 eq) in THE (1 mL)/H₂O (1 mL) was added prop-2-enoyl chloride (16.3 mg, 180.3 umol, 14.7 uL, 1 eq) at 0° C. The mixture was stirred at 0° C. for 1 hr. LC-MS showed desired compound was detected. The reaction mixture was concentrated under reduced pressure to remove solvent. The residue was purified by prep-HPLC (TFA condition) column: Phenomenex Luna 80×30 mm×3 um; mobile phase: [water(FA)-ACN]; B %: 3%-35%, 8 min then purified by prep-HPLC (neutral condition) column: Waters Xbridge BEH C18 100×30 mm×10 um; mobile phase: [water(NH₄HCO₃)-ACN]; B %: 30%-65%, 8 min to give 1-(3-hydroxycyclobutyl)-7-[4-(4-methylpiperazin-1-yl)anilino]-S-[(4S)-8-methyl-1-prop-2-enoyl-3,4-dihydro-2H-quinolin-4-yl]-4H-pyrimido[4,5-d]pyrimidin-2-one (14.7 mg, 24.1 umol, 13.4% yield, 100% purity) as a white solid.

### Procedure for Preparation of Compound 151

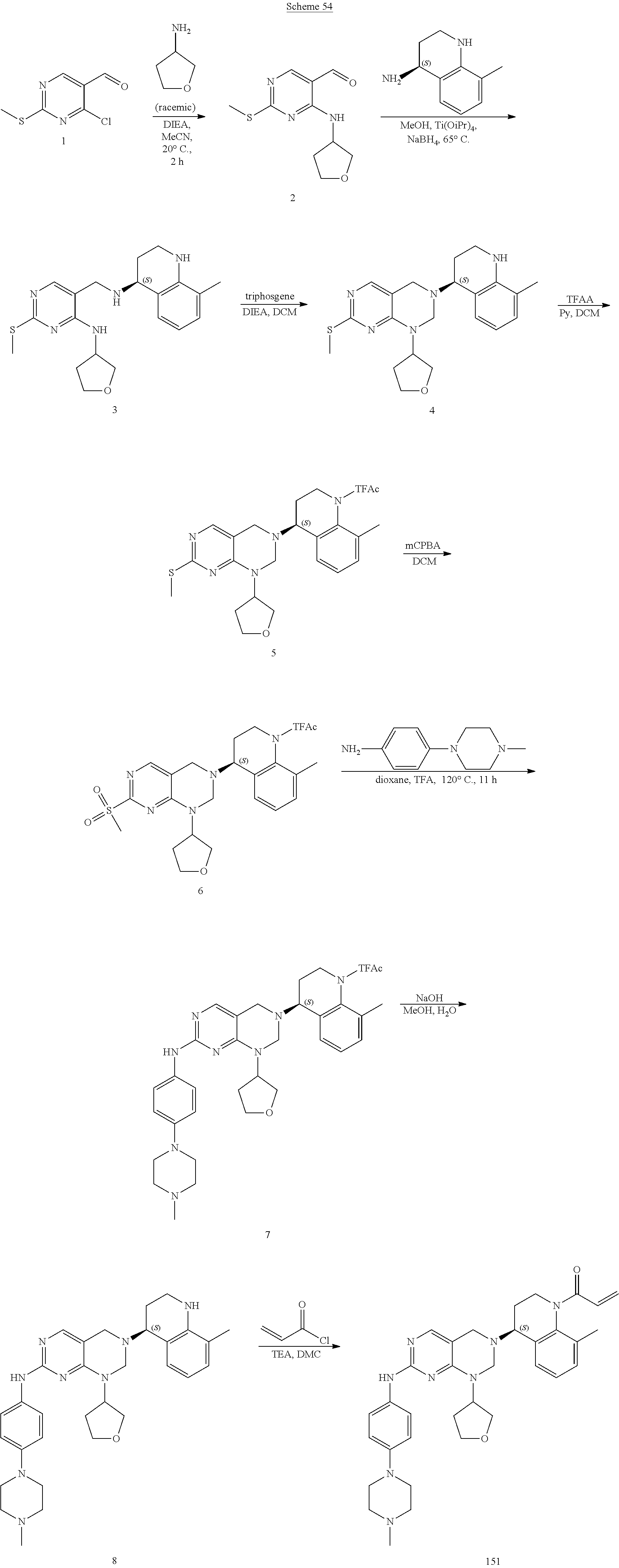

To a solution of tetrahydrofuran-S-amine (2 g, 22.9 mmol, 1 eq) in CH₃CN (120 mL) was added 4-chloro-2-methylsulfanyl-pyrimidine-5-carbaldehyde (4.3 g, 22.9 mmol, 1 eq) and DIEA (5.9 g, 45.9 mmol, 8.0 mL, 2 eq) to adjust pH to 8-9. The mixture was stirred at 20° C. for 2 hr. LC-MS showed desired mass was detected. The residue was diluted with H₂O 100 mL and extracted with Ethyl acetate 150 mL (50 mL×3), dried over Na₂SO₄, filtered and concentrated under reduced pressure to give a residue. The residue was purified by flash silica gel chromatography (20 g Silica Flash Column, Eluent of 0-20% Ethyl acetate/Petroleum ether gradient @ 100 mL/min) to give 2-methylsulfanyl-4-(tetrahydrofuran-S-ylamino)pyrimidine-5-carbaldehyde (4.0 g, 15.9 mmol, 69.3% yield, 95% purity) as yellow oil.

To a solution of 2-methylsulfanyl-4-(tetrahydrofuran-S-ylamino)pyrimidine-5-carbaldehyde (1.8 g, 7.8 mmol, 1 eq) in MeOH (50 mL) was added (4S)-8-methyl-1,2,3,4-tetrahydroquinolin-4-amine (1.2 g, 7.8 mmol, 1 eq) and Ti(i-PrO)₄ (6.6 g, 23.4 mmol, 6.9 mL, 3 eq). The mixture was stirred at 65° C. for 12 hr. NaBH₄ (887.8 mg, 23.4 mmol, 3 eq) was added at 0° C. The mixture was stirred at 25° C. for 2 hr. LC-MS showed desired mass was detected. The reaction mixture was diluted with NH₄Cl 100 mL and extracted with Dichloromethane 60 mL (20 mL×3), dried over Na₂SO₄, filtered and concentrated under reduced pressure to give a residue. The residue was purified by flash silica gel chromatography (40 g Silica Flash Column, Eluent of 0-100% Ethyl acetate/Petroleum ether gradient @100 mL/min) to give (4S)-8-methyl-N-[[2-methylsulfanyl-4-(tetrahydrofuran-S-ylamino)pyrimidin-5-yl]methyl]-1,2,3,4-tetrahydroquinolin-4-amine (970 mg, 2.2 mmol, 29.2% yield, 91% purity) as a white solid.

To a solution of (4S)-8-methyl-N-[[2-methylsulfanyl-4-(tetrahydrofuran-S-ylamino)pyrimidin-5-yl]methyl]-1,2,3,4-tetrahydroquinolin-4-amine (970 mg, 2.5 mmol, 1 eq) in DCM (15 mL) was added DIEA (975.5 mg, 7.55 mmol, 1.3 mL, 3 eq) and bis(trichloromethyl) carbonate (223.9 mg, 754.8 umol, 0.3 eq) at 0° C. The mixture was stirred at 0° C. for 2 hr. LC-MS showed desired mass was detected. The residue was diluted with NaHCO₃ 30 mL and extracted with Dichloromethane (30 mL×3). The combined organic layers were washed with brine (30 mL×3), dried over Na₂SO₄, filtered and concentrated under reduced pressure to give 7-methylsulfanyl-S-[(4S)-8-methyl-1,2,3,4-tetrahydroquinolin-4-yl]-1-tetrahydrofuran-S-yl-4H-pyrimido[4,5-d]pyrimidin-2-one (1.08 g, crude) as a white solid.

To a solution of 7-methylsulfanyl-S-[(4S)-8-methyl-1,2,3,4-tetrahydroquinolin-4-yl]-1-tetrahydrofuran-S-yl-4H-pyrimido[4,5-d]pyrimidin-2-one (1.0 g, 2.6 mmol, 1 eq) in DCM (12 mL) was added Py (830.3 mg, 10.5 mmol, 847.3 uL, 4 eq) at 0° C. and then TFAA (826.8 mg, 3.9 mmol, 547.5 uL, 1.5 eq) was added. The mixture was stirred at 20° C. for 4.5 hr. LC-MS showed desired mass was detected. The residue was diluted with H₂O 40 mL and extracted with Dichloromethane 60 mL (20 mL×3), dried over Na₂SO₄, filtered and concentrated under reduced pressure to give 7-methylsulfanyl-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (1 g, crude) as a yellow solid.

To a solution of 7-methylsulfanyl-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (1.3 g, 2.6 mmol, 1 eq) in DCM (20 mL) was added m-CPBA (816.0 mg, 4.0 mmol, 85% purity, 1.5 eq) at 0° C. The resulting mixture was stirred at 25° C. for 2 hr. LC-MS showed desired mass was detected. The reaction mixture was quenched by addition Na₂SO₃ 40 mL, extracted with Dichloromethane 60 mL (20 mL×3). The combined organic layers were washed with NaHCO₃ 60 mL (20 mL×3), and dried over Na₂SO₄, filtered and concentrated under reduced pressure to give 7-methylsulfonyl-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (1.0 g, crude) as a yellow solid.

To a solution of 7-methylsulfonyl-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (200 mg, 370.7 umol, 1 eq) in dioxane (3 mL) was added 4-(4-methylpiperazin-1-yl)aniline (106.3 mg, 556.0 umol, 1.5 eq) and TFA (63.4 mg, 556.0 umol, 41.1 uL, 1.5 eq). The mixture was stirred at 120° C. for 5 hr. LC-MS showed the reactant remained. 4-(4-methylpiperazin-1-yl)aniline (70.9 mg, 370.7 umol, 1 eq) and TFA (42.2 mg, 370.7 umol, 27.4 uL, 1 eq) was added. The mixture was stirred at 120° C. for another 6 hr. LC-MS showed desired mass was detected. The reaction mixture was concentrated under reduced pressure to remove solvent. The residue was purified by prep-HPLC (FA condition, column: Phenomenex luna C18 100×40 mm×5 um; mobile phase: [water(FA)-ACN];B %: 15%-45%, 8 min) to give 7-[4-(4-methylpiperazin-1-yl)anilino]-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (836.8 mg, 1.2 mmol, 66.6% yield, 96% purity) as a yellow solid.

To a solution of 7-[4-(4-methylpiperazin-1-yl)anilino]-S-[(4S)-8-methyl-1-(2,2,2-trifluoroacetyl)-3,4-dihydro-2H-quinolin-4-yl]-1-tetrahydrofuran-S-yl-4H-pyrimido[4,5-d]pyrimidin-2-one (471.4 mg, 724.4 umol, 1 eq) in MeOH (6 mL) and H₂O (2 mL) was added NaOH (144.8 mg, 3.6 mmol, 5 eq). The mixture was stirred at 50° C. for 1 hr. LC-MS showed desired mass was detected. The reaction mixture was concentrated under reduced pressure to remove solvent. The residue was diluted with H₂O 20 mL and extracted with Chloroform: Isopropyl alcohol=10:1 30 mL (10 mL×3), dried over Na₂SO₄, filtered and concentrated under reduced pressure to give 7-[4-(4-methylpiperazin-1-yl)anilino]-S-[(4S)-8-methyl-1,2,3,4-tetrahydroquinolin-4-yl]-1-tetrahydrofuran-S-yl-4H-pyrimido[4,5-d]pyrimidin-2-one (377.3 mg, crude) as a yellow solid.

To a solution of 7-[4-(4-methylpiperazin-1-yl)anilino]-S-[(4S)-8-methyl-1,2,3,4-tetrahydroquinolin-4-yl]-1-tetrahydrofuran-S-yl-4H-pyrimido[4,5-d]pyrimidin-2-one (100 mg, 180.2 umol, 1 eq) in DCM (2.5 mL) was added TEA (54.7 mg, 540.8 umol, 75.2 uL, 3 eq). And then prop-2-enoyl chloride (16.3 mg, 180.2 umol, 14.7 uL, 1 eq) in DCM (0.1 mL) was added dropwise at 0° C. The mixture was stirred at 0° C. for 1 hr. LC-MS showed the reactant remained mostly. Prop-2-enoyl chloride (16.3 mg, 180.2 umol, 14.7 uL, 1 eq) in DCM (0.1 mL) was added dropwise at 0° C. The mixture was stirred at 0° C. for 1 hr. LC-MS showed desired mass was detected. The residue was purified by prep-HPLC (TFA condition, column: Phenomenex Luna C18 100×30 mm×5 um;mobile phase: [water(TFA)-ACN];B %: 1%-35%, 8 min) to give 7-[4-(4-methylpiperazin-1-yl)anilino]-S-[(4S)-8-methyl-1-prop-2-enoyl-3,4-dihydro-2H-quinolin-4-yl]-1-tetrahydrofuran-S-yl-4H-pyrimido[4,5-d]pyrimidin-2-one (103.9 mg, 169.1 umol, 46.9% yield, 99% purity) as a yellow solid.

### Procedure for Preparation of Compound 153

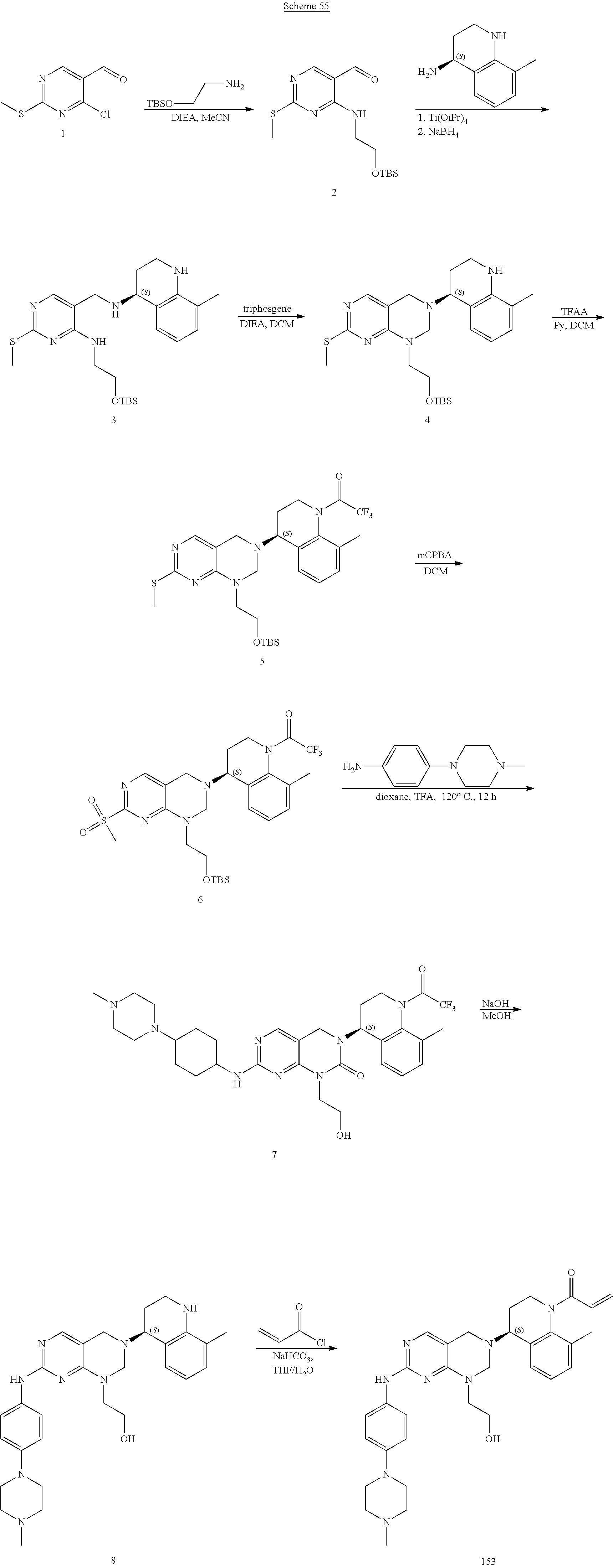

To a solution of 4-chloro-2-methylsulfanyl-pyrimidine-5-carbaldehyde (3 g, 15.9 mmol, 1 eq) in ACN (40 mL) was added DIEA (6.1 g, 47.7 mmol, 8.3 mL, 3 eq) and 2-[tert-butyl(dimethyl)silyl]oxyethanamine (3.3 g, 19.0 mmol, 1.2 eq). The mixture was stirred at 40° C. for 12 hr. LCMS showed the desired compound was detected. The reaction mixture was filtered and the filtrate dried in vacuum to give crude product. The reaction mixture was partitioned between H₂O (40 mL) and EtoAc (40 mL). The water phase was separated, extracted with EtoAc (40 mL×3), dried over Na₂SO₄, filtered and concentrated under reduced pressure to give a residue. The residue was purified by flash silica gel chromatography (40 g Flash Column, Eluent of 0-70% Ethyl acetate/Petroleum ether gradient @80 mL/min) to yield 4-((2-((tert-butyldimethylsilyl)oxy)ethyl)amino)-2-(methylthio)pyrimidine-5-carbaldehyde (3.1 g, 9.4 mmol, 59.5% yield) obtained as yellow oil.

To a solution of 4-((2-((tert-butyldimethylsilyl)oxy)ethyl)amino)-2-(methylthio)pyrimidine-5-carbaldehyde (1 g, 3.0 mmol, 1 eq) in MeOH (20 mL) was added Ti(i-PrO)₄ (1.7 g, 6.1 mmol, 1.8 mL, 2 eq) and AcOH (916.7 mg, 15.2 mmol, 873.0 uL, 5 eq) and (4S)-8-methyl-1,2,3,4-tetrahydroquinolin-4-amine (495.3 mg, 3.0 mmol, 1 eq). The mixture was stirred at 50° C. for 12 hr. NaBH₃CN (575.6 mg, 9.1 mmol, 3 eq) was added to the mixture. The mixture was stirred at 25° C. for 2 hr. LC-MS showed the desired compound was detected. The reaction mixture was filtered and the filtrate dried in vacuum to give crude product. The reaction mixture was partitioned between NaHCO₃ (20 mL) and EtoAc (20 mL). The water phase was separated, extracted with EtOAc (20 mL×3), dried over Na₂SO₄, filtered and concentrated under reduced pressure to give a residue. The residue was purified by flash silica gel chromatography (20 g Flash Silica Column, Eluent of 0-50% Petroleum ether/Ethyl acetate gradient @ 120 mL/min) to yield (S)—N-((4-((2-((tert-butyldimethylsilyl)oxy)ethyl)amino)-2-(methylthio)pyrimidin-5-yl)methyl)-8-methyl-1,2,3,4-tetrahydroquinolin-4-amine(1 g, 2.1 mmol, 69.1% yield) obtained as yellow oil.

To a solution of (S)—N-((4-((2-((tert-butyldimethylsilyl)oxy)ethyl)amino)-2-(methylthio)pyrimidin-5-yl)methyl)-8-methyl-1,2,3,4-tetrahydroquinolin-4-amine (1 g, 2.1 mmol, 1 eq) in DCM (20 mL) was added DIEA (1.0 g, 8.4 mmol, 1.4 mL, 4 eq) and triphosgene (250.5 mg, 844.3 umol, 0.4 eq) at 0° C. The mixture was stirred at 0° C. for 1 hr. LC-MS showed the desired compound was detected. The reaction mixture was diluted with NaHCO₃ (20 mL). The combined organic layers were washed with DCM (20 mL×3), dried over Na₂SO₄, filtered and concentrated under reduced pressure to give a residue to give (S)-1-(2-((tert-butyldimethylsilyl)oxy)ethyl)-S-(8-methyl-1,2,3,4-tetrahydroquinolin-4-yl)-7-(methylthio)-3,4-dihydropyrimido[4,5-d]pyrimidin-2(1H)-one (860 mg, crude) obtained as yellow oil.

To a solution of (S)-1-(2-((tert-butyldimethylsilyl)oxy)ethyl)-S-(8-methyl-1,2,3,4-tetrahydroquinolin-4-yl)-7-(methylthio)-3,4-dihydropyrimido[4,5-d]pyrimidin-2(1H)-one (860 mg, 1.7 mmol, 1 eq) in DCM (15 mL) was added Py (544.4 mg, 6.8 mmol, 555.6 uL, 4 eq) and TFAA (542.1 mg, 2.5 mmol, 359.0 uL, 1.5 eq) at 0° C. The mixture was stirred at 25° C. for 12 hr. LC-MS showed desired compound was detected. The residue was diluted with H₂O (10 mL) and extracted with DCM (10 mL×3). The combined organic layers were washed with brine (10 mL×1), dried over Na₂SO₄, filtered and concentrated under reduced pressure to give a residue. The residue was purified by flash silica gel chromatography (12 g Silica Flash Column, Eluent of 0-70% Ethyl acetate/Petroleum ether gradient @ 100 mL/min) to give (S)-1-(2-((tert-butyldimethylsilyl)oxy)ethyl)-S-(8-methyl-1-(2,2,2-trifluoroacetyl)-1,2,3,4-tetrahydroquinolin-4-yl)-7-(methylthio)-3,4-dihydropyrimido[4,5-d]pyrimidin-2(1H)-one(500 mg, 839.28 umol, 48.77% yield) obtained as a white solid.

To a solution of (S)-1-(2-((tert-butyldimethylsilyl)oxy)ethyl)-S-(8-methyl-1-(2,2,2-trifluoroacetyl)-1,2,3,4-tetrahydroquinolin-4-yl)-7-(methylthio)-3,4-dihydropyrimido[4,5-d]pyrimidin-2(1H)-one (500 mg, 839.2 umol, 1 eq) in DCM (10 mL) was added m-CPBA (306.7 mg, 1.5 mmol, 85% purity, 1.8 eq) at 0° C. The mixture was stirred at 25° C. for 2 hr.

LC-MS showed the desired compound was detected. The reaction mixture was partitioned between Na₂SO₃ (10 mL) and dichloromethane (10×3 mL). The organic phase was separated, washed with NaHCO₃ (10 mL×3), dried over Na₂SO₄, filtered and concentrated under reduced pressure to give (S)-1-(2-((tert-butyldimethylsilyl)oxy)ethyl)-S-(8-methyl-1-(2,2,2-trifluoroacetyl)-1,2,3,4-tetrahydroquinolin-4-yl)-7-(methylsulfonyl)-3,4-dihydropyrimido[4,5-d]pyrimidin-2(1H)-one (470 mg, crude) obtained as yellow oil.

To a solution of 1-[2-[tert-butyl(dimethyl)silyl]oxyethyl]-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (0.2 g, 318.6 umol, 1 eq) in dioxane (3 mL) was added TFA (43.5 mg, 382.3 umol, 28.3 uL, 1.2 eq) and 4-(4-methylpiperazin-1-yl)aniline (73.1 mg, 382.3 umol, 1.2 eq). The mixture was stirred at 120° C. for 12 hr. LC-MS showed the desired compound was detected. The reaction mixture was concentrated under reduced pressure to remove solvent. The residue was purified by prep-HPLC ((NH4HCO3 condition) column: Waters Xbridge BEH C18 100*30 mm*10 um; mobile phase: [water(NH₄HCO₃)-ACN];B %: 35%-55%, 8 min to give compound 1-(2-hydroxyethyl)-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (73 mg, 116.86 umol, 36.68% yield, n/a purity) obtained as brown oil.

To a solution of 1-(2-hydroxyethyl)-7-[4-(4-methylpiperazin-1-yl)anilino]-S-[(4S)-8-methyl-1-(2,2,2-trifluoroacetyl)-3,4-dihydro-2H-quinolin-4-yl]-4H-pyrimido[4,5-d]pyrimidin-2-one (73 mg, 116.8 umol, 1 eq) in H2O (1 mL) was added MeOH (3 mL) and NaOH (23.3 mg, 584.3 umol, 5 eq). The mixture was stirred at 50° C. for 2 hr. LC-MS showed the desired compound was detected. The reaction mixture was partitioned between H₂O (5 mL) and DCM: i-PrOH=3:1 (5 mL). The water phase was separated, extracted with DCM: i-PrOH=3:1 (5 mL×3), dried over Na2SO₄, filtered and concentrated under reduced pressure to give 1-(2-hydroxyethyl)-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (75 mg, crude) was obtained as yellow oil.

¹H NMR (400 MHz, DMSO-d₆) δ=9.23-9.15 (m, 1H), 7.95 (s, 1H), 7.55 (br d, J=9.0 Hz, 2H), 6.86 (br d, J=9.0 Hz, 3H), 6.68 (br d, J=7.8 Hz, 1H), 6.43 (t, J=7.5 Hz, 1H), 5.64 (br dd, J=5.8, 9.6 Hz, 1H), 5.22 (br s, 1H), 4.74 (t, J=5.3 Hz, 1H), 4.34 (d, J=4.1 Hz, 3H), 4.13-4.09 (m, 2H), 3.83-3.73 (m, 3H), 3.67-3.59 (m, 2H), 3.09-3.00 (m, 4H), 2.47-2.42 (m, 4H), 2.21 (s, 3H), 2.03 (s, 3H)

To a solution of 1-(2-hydroxyethyl)-7-[4-(4-methylpiperazin-1-yl)anilino]-S-[(4S)-8-methyl-1,2,3,4-tetrahydroquinolin-4-yl]-4H-pyrimido[4,5-d]pyrimidin-2-one (65 mg, 122.9 umol, 1 eq) in THE (1 mL) and H₂O (0.2 mL) was added NaHCO₃ (30.9 mg, 368.8 umol, 14.3 uL, 3 eq) and prop-2-enoyl chloride (11.1 mg, 122.9 umol, 10.0 uL, 1 eq) at 0° C. The mixture was stirred at 0° C. for 30 min. LC-MS showed the desired compound was detected. The reaction mixture was filtered and the filtrate dried in vacuum to give crude product. The residue was purified by prep-HPLC (FA condition; column: Phenomenex luna C18 100×40 mmx 5 um; mobile phase: [water(FA)-ACN];B %: 5%-35%, 8 min) to give 1-(2-hydroxyethyl)-7-[4-(4-methylpiperazin-1-yl)anilino]-S-[(4S)-8-methyl-1-prop-2-enoyl-3,4-dihydro-2H-quinolin-4-yl]-4H-pyrimido[4,5-d]pyrimidin-2-one (100% purity) (4 mg) as a white solid.

### Procedure for Preparation of Compound 154

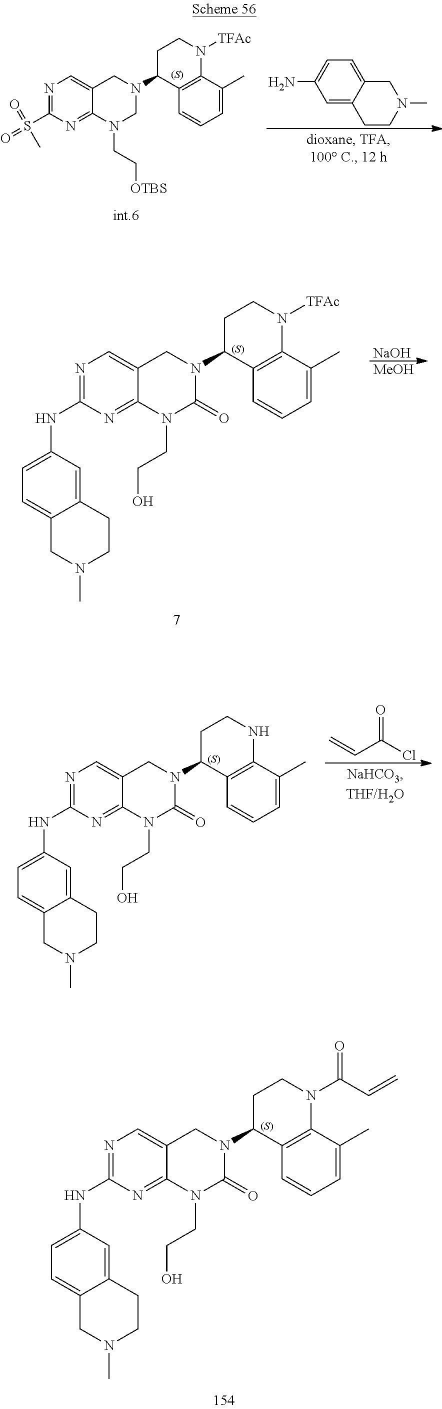

To a solution of 1-[2-[tert-butyl(dimethyl)silyl]oxyethyl]-7-methylsulfonyl-S-[(4S)-8-methyl-1-(2,2,2-trifluoroacetyl)-3,4-dihydro-2H-quinolin-4-yl]-4H-pyrimido[4,5-d]pyrimidin-2-one (50 mg, 79.65 umol, 1 eq) in dioxane (0.5 mL) was added TFA (10.9 mg, 95.5 umol, 7 μL, 1.2 eq) and 2-methyl-3,4-dihydro-1H-isoquinolin-6-amine (36.4 mg, 224.6 umol, 2.8 eq). The mixture was stirred at 80° C. for 12 hr. LC-MS showed the desired compound was detected. The reaction mixture was filtered and the filtrate dried in vacuum to give crude product. The residue was purified by prep-HPLC (FA condition, Phenomenex Luna C18 100×30 mm×5 um;mobile phase: [water(FA)-ACN];B %: 15%-50%, 8 min) to give 1-(2-hydroxyethyl)-7-[(2-methyl-3,4-dihydro-1 H-isoquinolin-6-yl)amino]-S-[(4S)-8-methyl-1-(2,2,2-trifluoroacetyl)-3,4-dihydro-2H-quinolin-4-yl]-4H-pyrimido[4,5-d]pyrimidin-2-one obtained as a white solid(30 mg).

To a solution of 1-(2-hydroxyethyl)-7-[(2-methyl-3,4-dihydro-1H-isoquinolin-6-yl)amin o]—S-[(4S)-8-methyl-1-(2,2,2-trifluoroacetyl)-3,4-dihydro-2H-quinolin-4-yl]-4H-pyrimido[4,5-d]p yrimidin-2-one (30 mg, 50.3 umol, 1eq) in H₂O (1 mL) was added MeOH (3 mL) and NaOH (10 mg, 251.8 umol, 5 eq). The mixture was stirred at 50° C. for 2 hr. LC-MS showed the desired compound was detected. The reaction mixture was partitioned between H₂O (5 mL) and DCM:i-PrOH=3:1(5 mL). The water phase was extracted with DCM:i-PrOH=3:1 (5 mL×3). The combined organic layers were dried over Na₂SO₄, filtered and concentrated under reduced pressure to give 1-(2-hydroxyethyl)-7-[(2-methyl-3,4-dihydro-1H-isoquinolin-6-yl)amino]-S-[(4S)-8-methyl-1,2,3,4-tetrahydroquinolin-4-yl]-4H-pyrimido[4,5-d]pyrimidin-2-one (35 mg, cru de) as a yellow oil.

To a solution of 1-(2-hydroxyethyl)-7-[(2-methyl-3,4-dihydro-1H-isoquinolin-6-yl) amino]-S—[(4S)-8-methyl-1,2,3,4-tetrahydroquinolin-4-yl]-4H-pyrimido [4,5-d]pyrimidin-2-one (50 mg, 100 umol, 1 eq) in THE (1 mL) and H₂O (0.2 mL) was added NaHCO₃ (25.2 mg, 300.2 umol, 11.6 uL, 3 eq) and prop-2-enoyl chloride (9 mg, 100 umol, 8.1 uL, 1 eq) at 0° C. The mixture was stirred at 0° C. for 30 min. LC-MS showed the desired compound was detected. The residue was purified by prep-HPLC (TFA condition; column: Phenomenex Luna C18 100×30 mm×5 um;mobile phase: [water(TFA)-ACN];B %: 10%-40%, 8 min to give 1-(2-hydroxyethyl)-7-[(2-methyl-3,4-dihydro-1 H-isoquinolin-6-yl)amino]-S-[(4S)-8-methyl-1-prop-2-enoyl-3,4-dihydro-2H-quinolin-4-yl]-4H-pyrimido[4,5-d]pyrimidin-2-one (6 mg, 10.84 umol, 5.41% yield, 100% purity) was obtained as a yellow solid.

### Procedure for Preparation of Compound 157

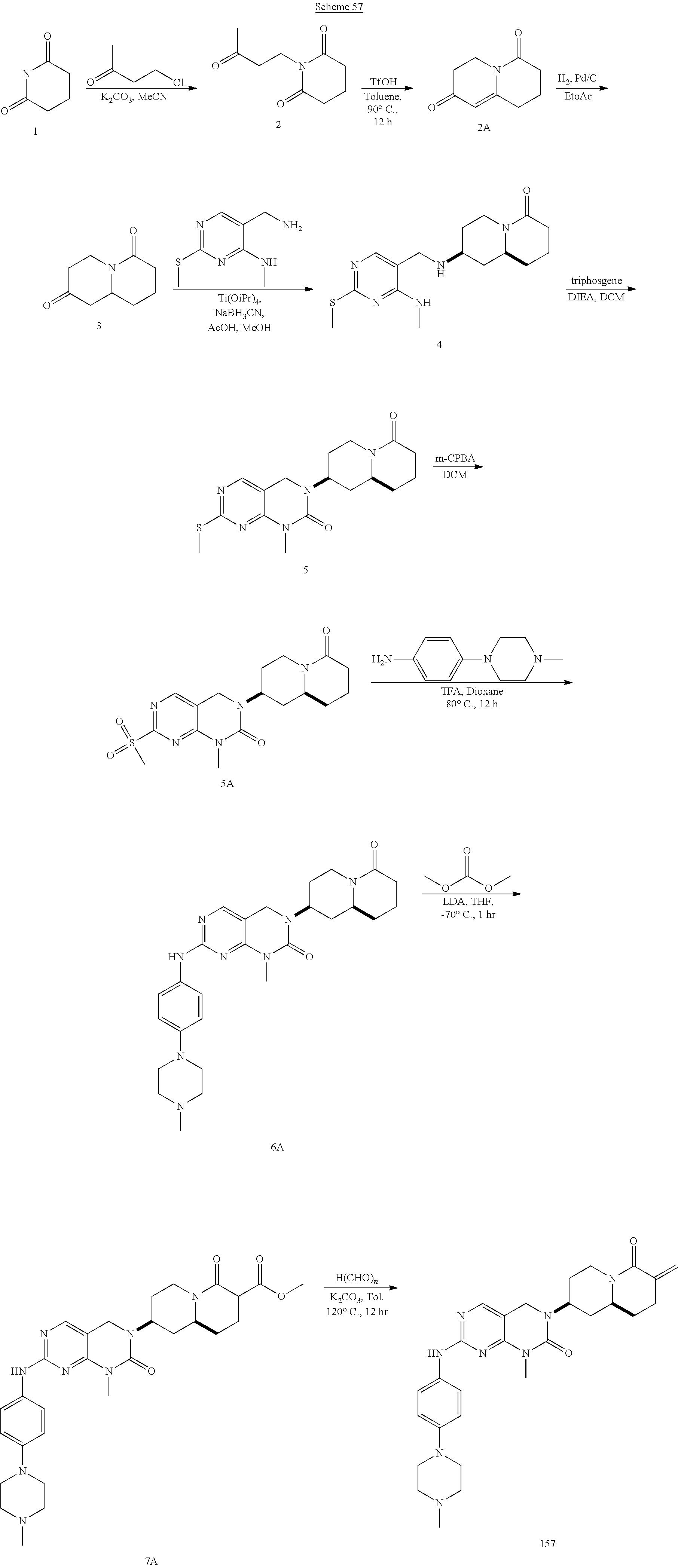

To a solution of piperidine-2,6-dione (10 g, 88.4 mmol, 1 eq) in MeCN (150 mL) was added K₂CO₃ (36.7 g, 265.2 mmol, 3eq) and 4-chlorobutan-2-one (9.4 g, 88.4 mmol, 1eq). The mixture was stirred at 60° C. for 12 hr. LC-MS showed desired compound was detected. The mixture was filtered and the filtrate was concentrated to get a compound 1-(3-oxobutyl)piperidine-2,6-dione (16 g, crude) as a white solid.

To a solution of 1-(3-oxobutyl)piperidine-2,6-dione (16 g, 87.3 mmol, 1 eq) in Tol. (160 mL) was added TfOH (13.11 g, 87.33 mmol, 7.71 mL, 1eq). The mixture was stirred at 90° C. for 12 hr. TLC showed desired compound was detected. The reaction mixture was quenched by addition Sat. NaHCO₃ 50 mL, and then extracted with ethyl acetate (50 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=3/1 to 1/1) to give compound 4,7,8,9-tetrahydro-3H-quinolizine-2,6-dione (5 g, 30.3 mmol, 34.66% yield) as an orange oil

A mixture of 4,7,8,9-tetrahydro-3H-quinolizine-2,6-dione (5 g, 30.3 mmol, 1 eq), Pd/C (1 g, 10% purity) in EtOAc 100 mL was degassed and purged with H₂ for 3 times, and then the mixture was stirred at 20° C. for 8 hr under H₂ atmosphere (15 psi). TLC showed desired compound was detected. The mixture was filtered and the filtrate was concentrated to get compound 3,4,7,8,9,9a-hexahydro-1 H-quinolizine-2,6-dione (4.3 g, crude) as a yellow oil.

To a solution of 3,4,7,8,9,9a-hexahydro-1H-quinolizine-2,6-dione (2 g, 12.0 mmol, 1 eq) 5-(aminomethyl)-N-methyl-2-methylsulfanyl-pyrimidin-4-amine (2.0 g, 10.8 mmol, 0.9 eq) in MeOH (30 mL) was added Ti(i-PrO)₄ (10.2 g, 35.9 mmol, 10.6 mL, 3 eq) and AcOH (3.6 g, 59.8 mmol, 3.4 mL, 5 eq), the mixture was stirred at 25° C. for 12 hr, then NaBH₃CN (2.26 g, 35.88 mmol, 3 eq) was added. The mixture was stirred at 25° C. for 3 hr. LC-MS showed desired compound was detected. The mixture was filtered and the filtrate was quenched by Sat·NaHCO₃ 20 mL and then extracted by ethyl acetate (30 mL×3), the combined organic layers was dried by Na₂SO₄, filtered and the filtrate was concentrated to get a residue. The residue was purified by column chromatography (SiO₂, Petroleum ether/Ethyl acetate=3/1 to 0/1) to give compound 8-[[4-(methylamino)-2-methylsulfanyl-pyrimidin-5-yl]methylamino]-1,2,3,6,7,8,9,9a-octahydroquinolizin-4-one (3 g, 8.9 mmol, 74.76% yield) as a white solid

To a solution of 8-[[4-(methylamino)-2-methylsulfanyl-pyrimidin-5-yl]methylamino]-1,2,3,6,7,8,9,9a-octahydroquinolizin-4-one (3 g, 8.9 mmol, 1 eq) and DIEA (6.9 g, 53.7 mmol, 9.4 mL, 6 eq) in DCM (40 mL) was added triphosgene (2.1 g, 7.2 mmol, 0.8 eq). The mixture was stirred at 25° C. for 2 hr. The mixture was stirred at 25° C. for 1 hr. LC-MS showed desired compound was detected. The mixture was quenched by NaHCO₃ 100 mL, then the mixture was extracted by DCM (20 mL×3), the combined organic layers was washed by brine, then dried by Na₂SO₄, filtered and the filtrate was concentrated to get a residue. The residue was purified by column chromatography (SiO₂, Petroleum ether/Ethyl acetate=3/1 to 1/1) to give compound 1-methyl-7-methylsulfanyl-S-(6-oxo-1,2,3,4,7,8,9,9a-octahydroquinolizin-2-yl)-4H-pyrimido[4,5-d]pyrimidin-2-one (3 g, 7.1 mmol, 79.54% yield, 85.70% purity) as a yellow solid.

To a solution of 1-methyl-7-methylsulfanyl-S-(6-oxo-1,2,3,4,7,8,9,9a-octahydroquinolizin-2-yl)-4H-pyrimido[4,5-d]pyrimidin-2-one (400 mg, 1.1 mmol, 1 eq) in DCM (5 mL) was added m-CPBA (404.4 mg, 2.0 mmol, 85% purity, 1.8 eq). The mixture was stirred at 25° C. for 12 hr. LC-MS showed desired compound was detected. The reaction mixture was partitioned between Na₂SO₃ 10 mL and dichloromethane (5×3 mL). The organic phase was separated, washed with NaHCO₃ (10 mL×3), dried over Na₂SO₄, filtered and concentrated under reduced pressure to give 1-methyl-7-methylsulfonyl-S-(6-oxo-1,2,3,4,7,8,9,9a-octahydroquinolizin-2-yl)-4H-pyrimido[4,5-d]pyrimidin-2-one (400 mg, crude) as a white solid.

To a solution of 1-methyl-7-methylsulfonyl-S-(6-oxo-1,2,3,4,7,8,9,9a-octahydroquinolizin-2-yl)-4H-pyrimido[4,5-d]pyrimidin-2-one (300 mg, 762.5 umol, 1 eq) in dioxane (2 mL) was added TFA (104.3 mg, 915.0 umol, 67.7 uL, 1.2 eq) and 4-(4-methylpiperazin-1-yl)aniline (145.8 mg, 762.5 umol, 1 eq). The mixture was stirred at 80° C. for 12 hr. LC-MS showed desired compound was detected. The mixture was concentrated to get a residue. The residue was purified by prep-HPLC (TFA condition; column: Phenomenex Luna C18 100×30 mm×5 um;mobile phase: [water(TFA)-ACN];B %: 1%-30%, 8 min). to give compound 1-methyl-7-[4-(4-methylpiperazin-1-yl)anilino]-S-(6-oxo-1,2,3,4,7,8,9,9a-octahydroquinolizin-2-yl)-4H-pyrimido[4,5-d]pyrimidin-2-one (270 mg, 535.1 umol, 35.09% yield) as a black solid.

To a solution of 1-methyl-7-[4-(4-methylpiperazin-1-yl)anilino]-S-(6-oxo-1,2,3,4,7,8,9,9a-octahydroquinolizin-2-yl)-4H-pyrimido[4,5-d]pyrimidin-2-one (40 mg, 79.3 umol, 1 eq) in THE (1 mL) was added LDA (2 M, 118.9 uL, 3 eq) at −70° C. under N₂, the mixture was stirred at −70° C. for 1 hr, then dimethyl carbonate (21.4 mg, 237.8 umol, 20.0 uL, 3 eq) was added, the mixture was stirred at −70° C. for 1 hr. LC-MS showed desired compound was detected. the mixture was quenched by Sat, NH₄Cl 1 mL and extracted by ethyl acetate (0.5 mL×3), the combined organic layers was dried by Na₂SO₄, filtered and the filtrate was concentrated to get the crude product methyl 8-[1-methyl-7-[4-(4-methylpiperazin-1-yl)anilino]-2-oxo-4H-pyrimido[4,5-d]pyrimidin-S-yl]-4-oxo-1,2,3,6,7,8,9,9a-octahydroquinolizine-S-carboxylate (120 mg, crude) as a brown solid used into the next step without further purification

To a solution of methyl 8-[1-methyl-7-[4-(4-methylpiperazin-1-yl)anilino]-2-oxo-4H-pyrimido[4,5-d]pyrimidin-S-yl]-4-oxo-1,2,3,6,7,8,9,9a-octahydroquinolizine-S-carboxylate (80 mg, 142.2 umol, 1 eq) in Tol. (2 mL) was added paraformaldehyde (136.5 mg, 1.4 mmol, 10 eq) and 18-crown-6 (7.5 mg, 28.4 umol, 0.2 eq) and K₂CO₃ (59.0 mg, 426.5 umol, 3 eq). The mixture was stirred at 120° C. for 12 hr. LC-MS showed desired compound was detected. The mixture was concentrated to get a residue. The residue was purified by prep-HPLC (neutral condition; column: Waters Xbridge BEH C18 100×30 mm×10 um; mobile phase: [water(NH₄HCO₃)-ACN];B %: 25%-55%, 8 min), then purified by prep-HPLC (TFA condition. column: Phenomenex C18 75×30 mm×3 um;mobile phase: [water(TFA)-ACN];B %: 1%-30%, 8 min) to give compound 1-methyl-S-(7-methylene-6-oxo-2,3,4,8,9,9a-hexahydro-1 H-quinolizin-2-yl)-7-[4-(4-methylpiperazin-1-yl)anilino]-4H-pyrimido[4,5-d]pyrimidin-2-one (4.9 mg, 9.5 umol, 6.67% yield, 100% purity) as a white solid.

### Procedure for Preparation of Compound 158

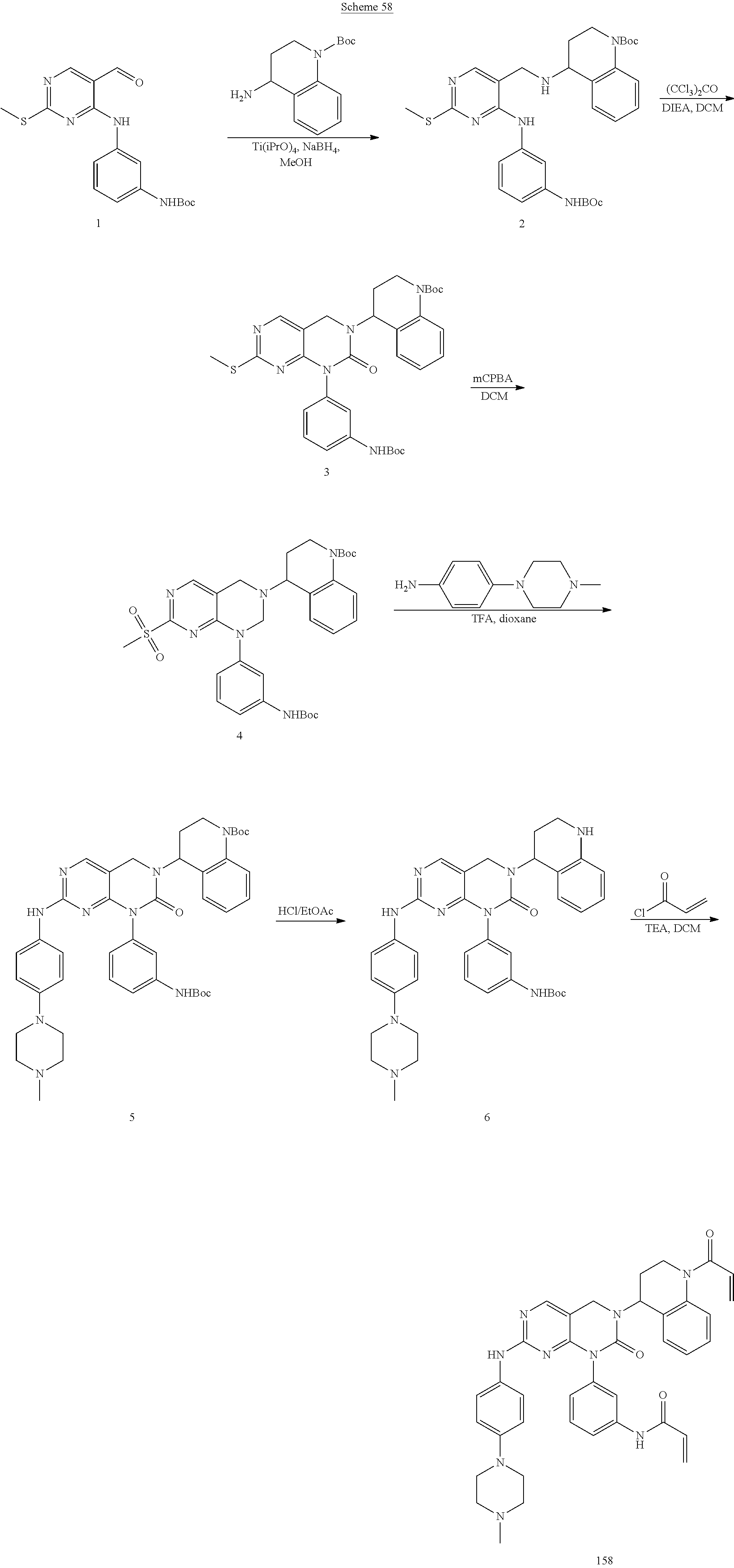

To a solution of tert-butyl N-[3-[(5-formyl-2-methylsulfanyl-pyrimidin-4-yl)amino]phenyl]carbamate (800 mg, 2.22 mmol, 1 eq) and tert-butyl 4-amino-3,4-dihydro-2H-quinoline-1-carboxylate (551.16 mg, 2.22 mmol, 1 eq) in MeOH (20 mL) was added tetraisopropoxytitanium (1.89 g, 6.66 mmol, 1.97 mL, 3 eq), stirred at 30° C. for 12 hr. NaBH₄ (503.83 mg, 13.32 mmol, 6 eq) was added to the reaction at 0° C. and stirred at 20° C. for 4 hr.

LC-MS showed desired compound was detected. The reaction mixture was concentrated under reduced pressure to remove solvent. The reaction mixture was diluted with NH₄Cl 50 mL and extracted with EtOAc 90 mL (30 mL×3). The combined organic layers were washed with brine 100 mL (50 mL×2), dried over Na₂SO₄, filtered and the filtrate was concentrated under reduced pressure to give a residue. The residue was purified by flash silica gel chromatography (ISCO®; 20 g SepaFlash® Silica Flash Column, Eluent of 0-60% Ethyl acetate/Petroleum ether gradient @ 120 mL/min) to give tert-butyl 4-[[4-[3-(tert-butoxycarbonylamino)anilino]-2-methylsulfanyl-pyrimidin-5-yl]methylamino]-3,4-dihydro-2H-quinoline-1-carboxylate (450 mg, 687.81 umol, 30.99% yield, 90.6% purity) as a yellow solid.

¹H NMR (400 MHz, CHLOROFORM-d) 5=9.66 (s, 1H), 7.96 (s, 1H), 7.87 (br d, J=8.6 Hz, 1H), 7.57-7.52 (m, 1H), 7.42 (br d, J=8.1 Hz, 1H), 7.25-7.20 (m, 2H), 7.15-7.04 (m, 2H), 6.79 (br s, 1H), 3.95-3.89 (m, 1H), 3.87 (s, 2H), 3.82-3.73 (m, 2H), 2.56 (s, 3H), 2.15-2.07 (m, 2H), 1.54 (s, 18H)

To a solution of tert-butyl 4-[[4-[3-(tert-butoxycarbonylamino)anilino]-2-methylsulfanyl-pyrimidin-5-yl]methylamino]-3,4-dihydro-2H-quinoline-1-carboxylate (420 mg, 708.56 umol, 1 eq) and DIEA (457.87 mg, 3.54 mmol, 617.08 uL, 5 eq) in DCM (5 mL) was added triphosgene (210.26 mg, 708.56 umol, 1 eq) at 0° C., the reaction was stirred at 25° C. for 12 hr. LC-MS showed desired compound was detected. The residue was diluted with NaHCO₃ 50 mL and extracted with DCM 60 mL (20 mL×3). The combined organic layers were washed with brine 40 mL (20 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0-100% Ethyl acetate/Petroleum ethergradient @ 120 mL/min) to give tert-butyl 4-[1-[3-(tert-butoxycarbonylamino)phenyl]-7-methylsulfanyl-2-oxo-4H-pyrimido[4,5-d]pyrimidin-S-yl]-3,4-dihydro-2H-quinoline-1-carboxylate (320 mg, 517.18 umol, 72.99% yield, 100% purity) as a yellow solid.

¹H NMR (400 MHz, DMSO-d6) 5=9.51 (s, 1H), 8.26 (s, 1H), 7.70 (d, J=8.4 Hz, 1H), 7.55 (br s, 1H), 7.39-7.30 (m, 2H), 7.27-7.18 (m, 2H), 7.11-7.05 (m, 1H), 6.96-6.92 (m, 1H), 5.63 (br t, J=8.1 Hz, 1H), 4.12-4.03 (m, 2H), 3.65-3.55 (m, 2H), 2.20-2.16 (m, 3H), 2.16-2.06 (m, 2H), 1.48 (d, J=8.2 Hz, 18H)

To a solution of tert-butyl 4-[1-[3-(tert-butoxycarbonylamino)phenyl]-7-methylsulfanyl-2-oxo-4H-pyrimido[4,5-d]pyrimidin-S-yl]-3,4-dihydro-2H-quinoline-1-carboxylate (320 mg, 517.18 umol, 1 eq) in DCM (3 mL) was added m-CPBA (189.00 mg, 930.92 umol, 85% purity, 1.8 eq) at 0° C. The mixture was stirred at 25° C. for 12 hr. LC-MS showed desired compound was detected. The reaction mixture was diluted with Na₂SO₃ 15 mL and extracted with DCM 15 mL (5 mL×3). The combined organic layers were washed with NaHCO₃ 10 mL (5 mL×2), dried over Na₂SO₄, filtered and the filtrate was concentrated under reduced pressure to give tert-butyl 4-[1-[3-(tert-butoxycarbonylamino)phenyl]-7-methylsulfonyl-2-oxo-4H-pyrimido[4,5-d]pyrimidin-S-yl]-3,4-dihydro-2H-quinoline-1-carboxylate (338 mg, crude) as a yellow solid.

¹H NMR (400 MHz, DMSO-d6) 5=9.56 (s, 1H), 8.64-8.57 (m, 1H), 7.71 (d, J=8.2 Hz, 1H), 7.66-7.59 (m, 1H), 7.39-7.33 (m, 2H), 7.30-7.22 (m, 2H), 7.12-7.05 (m, 1H), 7.01-6.92 (m, 1H), 5.64 (br t, J=8.1 Hz, 1H), 4.17-4.01 (m, 2H), 3.65-3.57 (m, 2H), 2.20-2.06 (m, 2H), 1.49 (d, J=10.6 Hz, 18H)

To a solution of tert-butyl 4-[1-[3-(tert-butoxycarbonylamino)phenyl]-7-methylsulfonyl-2-oxo-4H-pyrimido[4,5-d]pyrimidin-S-yl]-3,4-dihydro-2H-quinoline-1-carboxylate (110 mg, 169.04 umol, 1 eq) in dioxane (2 mL) was added 4-(4-methylpiperazin-1-yl)aniline (64.66 mg, 338.07 umol, 2 eq) and TFA (28.91 mg, 253.56 umol, 18.77 uL, 1.5 eq), the reaction was stirred at 120° C. for 12 hr. LC-MS showed desired compound was detected. The reaction mixture was concentrated under reduced pressure to remove solvent. The residue was purified by prep-HPLC (FA condition, column: Phenomenex Luna C18 75*30 mm*3 um;mobile phase: [water(FA)-ACN];B %: 20%-50%, 8 min) to give tert-butyl 4-[1-[3-(tert-butoxycarbonylamino)phenyl]-7-[4-(4-methylpiperazin-1-yl)anilino]-2-oxo-4H-pyrimido[4,5-d]pyrimidin-S-yl]-3,4-dihydro-2H-quinoline-1-carboxylate (140 mg, 183.75 umol, 36.23% yield) as a yellow solid. tert-butyl 4-[1-[3-(tert-butoxycarbonylamino)phenyl]-7-[4-(4-methylpiperazin-1-yl)anilino]-2-oxo-4H-pyrimido[4,5-d]pyrimidin-S-yl]-3,4-dihydro-2H-quinoline-1-carboxylate (140 mg, 183.75 umol, 1 eq) in HCl/EtOAc (2 mL, 4M) was stirred at 20° C. for 1 hr. LC-MS showed desired compound was detected. The reaction mixture was concentrated under reduced pressure to give 1-(3-aminophenyl)-7-[4-(4-methylpiperazin-1-yl)anilino]-S-(1,2,3,4-tetrahydroquinolin-4-yl)-4H-pyrimido[4,5-d]pyrimidin-2-one (134 mg, crude) as a yellow solid.

To a solution of 1-(3-aminophenyl)-7-[4-(4-methylpiperazin-1-yl)anilino]-S-(1,2,3,4-tetrahydroquinolin-4-yl)-4H-pyrimido[4,5-d]pyrimidin-2-one (130 mg, 217.34 umol, 1 eq, HCl) and TEA (65.98 mg, 652.02 umol, 90.75 uL, 3 eq) in DCM (3 mL) was dropwise added a DCM solution of prop-2-enoyl chloride (39.34 mg, 434.68 umol, 35.44 uL, 2 eq) at 0° C., the reaction was stirred at 0° C. for 20 min. LC-MS showed desired compound was detected. The reaction mixture was concentrated under reduced pressure to remove solvent. The residue was purified by prep-HPLC (FA condition, column: Phenomenex Luna C18 75*30 mm*3 um;mobile phase: [water(FA)-ACN];B %: 15%-45%, 8 min) to give N-[3-[7-[4-(4-methylpiperazin-1-yl)anilino]-2-oxo-S-(1-prop-2-enoyl-3,4-dihydro-2H-quinolin-4-yl)-4H-pyrimido[4,5-d]pyrimidin-1-yl]phenyl]prop-2-enamide (25.2 mg, 32.90 umol, 15.14% yield, 87.43% purity) as a white solid.

### Procedure for Preparation of Compound 159

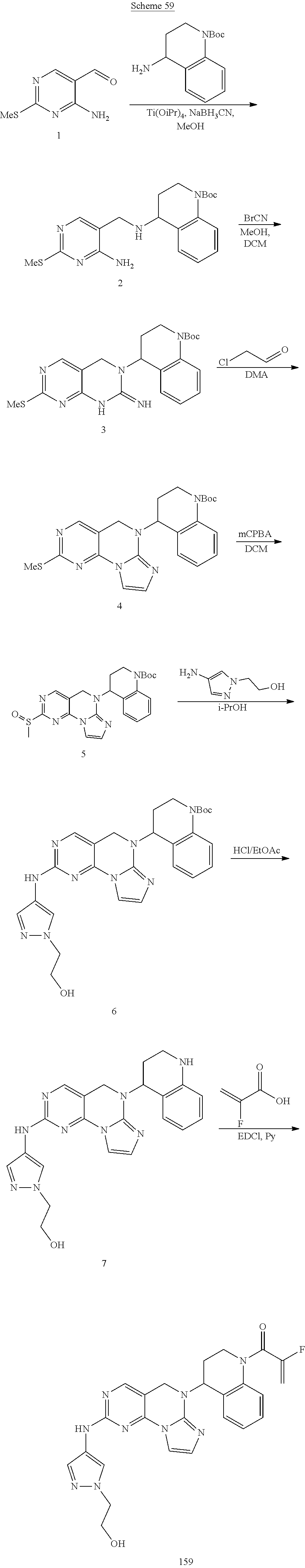

To a solution of 4-amino-2-methylsulfanyl-pyrimidine-5-carbaldehyde (1 g, 5.9 mmol, 1 eq) in MeOH (50 mL) was added AcOH (1.77 g, 29.6 mmol, 1.69 mL, 5 eq), tetraisopropoxytitanium (5.0 g, 17.7 mmol, 5.2 mL, 3 eq) and tert-butyl 4-amino-3,4-dihydro-2H-quinoline-1-carboxylate (1.5 g, 5.9 mmol, 1 eq). The mixture was stirred at 60° C. for 12 h.

Then NaBH₃CN (2.2 g, 35.5 mmol, 6 eq) was added to the mixture. The mixture was stirred at 60° C. for 3 h. LC-MS showed desired compound was detected. The residue was diluted with NaHCO₃ 100 mL and extracted with ethyl acetate (100 mL×3). The combined organic layers were washed with brine (70 mL×1), dried over Na₂SO₄, filtered and concentrated under reduced pressure to give a residue. The residue was purified by flash silica gel chromatography (40 g Silica Flash Column, Eluent of 0-100% Ethyl acetate/Petroleum ether gradient @ 120 mL/min). to give tert-butyl 4-[(4-amino-2-methylsulfanyl-pyrimidin-5-yl)methylamino]-3,4-dihydro-2H-quinoline-1-carboxylate (1 g, 2.5 mmol, 42.1% yield) as a white solid.

To a solution of tert-butyl 4-[(4-amino-2-methylsulfanyl-pyrimidin-5-yl)methylamino]-3,4-dihydro-2H-quinoline-1-carboxylate (1 g, 2.5 mmol, 1 eq) in MeOH (20 mL) was added bromo cyanide (395.7 mg, 3.7 mmol, 274.8 uL, 1.5 eq) in DCM (10 mL). The mixture was stirred at 50° C. for 12 h. LC-MS showed desired compound was detected. The mixture was quenched by H₂O 50 mL and then extracted by dichloromethane (50 mL×3). The organic layers was washed by brine (50 mL×2) and then dried by Na₂SO₄, filtered and the filtrate was concentrated to get tert-butyl 4-(2-imino-7-methylsulfanyl-1,4-dihydropyrimido[4,5-d]pyrimidin-S-yl)-3,4-dihydro-2H-quinoline-1-carboxylate (1 g, crude) as a yellow solid.

To a solution of tert-butyl 4-(2-imino-7-methylsulfanyl-1,4-dihydropyrimido[4,5-d]pyrimidin-S-yl)-3,4-dihydro-2H-quinoline-1-carboxylate (880 mg, 2.1 mmol, 1 eq) in DMA (10 mL) was added 2-chloroacetaldehyde (890.7 mg, 4.5 mmol, 730.1 uL, 40% aqueous solution, 2.2 eq), the reaction was stirred at 100° C. for 1 h. LC-MS showed desired compound was detected. The mixture was poured into water and then extracted by ethyl acetate (20 mL×3), the combined organic layers was washed by brine (10 mL×2), and then dried by Na₂SO₄, then filtered and the filtrate was concentrated to get a residue. The residue was purified by column chromatography (SiO₂, Petroleum ether/Ethyl acetate=10/1 to 1/1) to give tert-butyl 4-(12-methylsulfanyl-2,5,7,11,13-pentazatricyclo[7.4.0.02,6]trideca-1 (13), 3,5,9,11-pentaen-7-yl)-3,4-dihydro-2H-quinoline-1-carboxylate (700 mg, 1.5 mmol, 75.3% yield) as an orange oil.

To a solution of tert-butyl 4-(12-methylsulfanyl-2,5,7,11,13-pentazatricyclo[7.4.0.02,6]trideca-1(13), 3,5,9,11-pentaen-7-yl)-3,4-dihydro-2H-quinoline-1-carboxylate (600 mg, 1.3 mmol, 1 eq) in DCM (20 mL) was added m-CPBA (324.4 mg, 1.6 mmol, 85% purity, 1.2 eq). The mixture was stirred at 25° C. for 12 h. LC-MS showed desired compound was detected. The reaction mixture was partitioned between Na₂SO₃ 20 mL and dichloromethane (20 mL×3). The organic phase was separated, washed with NaHCO₃ (15 mL×3), dried over Na₂SO₄, filtered and concentrated under reduced pressure to give a residue. The residue was purified by column chromatography (SiO₂, Petroleum ether/Ethyl acetate=5/1 to 0/1) to give tert-butyl 4-(12-methylsulfinyl-2,5,7,11,13 pentazatricyclo[7.4.0.02,6] trideca-1(13), 3,5,9,11-pentaen-7-yl)-3,4-dihydro-2H-quinoline-1-carboxylate (550 mg, 1.1 mmol, 88.5% yield) as an orange oil.

To a solution of tert-butyl 4-(12-methylsulfinyl-2,5,7,11,13-pentazatricyclo[7.4.0.02,6]trideca-1(13), 3,5,9,11-pentaen-7-yl)-3,4-dihydro-2H-quinoline-1-carboxylate (150 mg, 321.5 umol, 1 eq) in i-PrOH (3 mL) was added 2-(4-aminopyrazol-1-yl)ethanol (49.1 mg, 385.8 umol, 1.2 eq). The mixture was stirred at 80° C. for 1 h. LC-MS showed desired compound was detected. The mixture was concentrated to get a residue.

The residue was purified by prep-HPLC (FA condition; column: Phenomenex Luna C18 75*30 mm*3 um; mobile phase: [water (FA)-ACN];B %: 10%-45%, 8 min) to give tert-butyl 4-[12-[[1-(2-hydroxyethyl)pyrazol-4-yl]amino]-2,5,7,11,13-pentazatricyclo[7.4.0.02,6] trideca-1(13), 3,5,9,11-pentaen-7-yl]-3,4-dihydro-2H-quinoline-1-carboxylate (70 mg, 132.1 umol, 41.1% yield) as a brown solid.

To a solution of tert-butyl 4-[12-[[1-(2-hydroxyethyl)pyrazol-4-yl]amino]-2,5,7,11,13-pentazatricyclo[7.4.0.02,6] trideca-1(13), 3,5,9,11-pentaen-7-yl]-3,4-dihydro-2H-quinoline-1-carboxylate (50 mg, 94.4 umol, 1 eq) in EtOAc (2 mL) was added HCl/EtOAc (1 mL, 4M).

The mixture was stirred at 20° C. for 1 h. LC-MS showed desired compound was detected.

The mixture was concentrated to get 2-[4-[[7-(1,2,3,4-tetrahydroquinolin-4-yl)-2,5,7,11,13-pentazatricyclo[7.4.0.02,6]trideca-1(13), 3,5,9,11-pentaen-12-yl]amino]pyrazol-1-yl]ethanol (45 mg, crude, HCl) as a yellow solid.

To a solution of 2-[4-[[7-(1,2,3,4-tetrahydroquinolin-4-yl)-2,5,7,11,13-pentazatricyclo[7.4.0.02,6]trideca-1(13), 3,5,9,11-pentaen-12-yl]amino]pyrazol-1-yl]ethanol (45 mg, 96.6 umol, 1 eq, HCl) in Pyridine (2 mL) was added EDCI (27.8 mg, 144.9 umol, 1.5 eq) and 2-fluoroprop-2-enoic acid (9.6 mg, 106.2 umol, 1.1 eq). The mixture was stirred at 50° C. for 12 hr. LC-MS showed desired compound was detected. The mixture was concentrated to get a residue. The residue was purified by prep-HPLC (FA condition column: Phenomenex Luna C18 75*30 mm*3 um;mobile phase: [water(FA)-ACN];B %: 1%-30%, 8 min) to give compound 2-fluoro-1-[4-[12-[[1-(2-hydroxyethyl)pyrazol-4-yl]amino]-2,5,7,11,13-pentazatricyclo[7.4.0.02,6] trideca-1(13), 3,5,9,11-pentaen-7-yl]-3,4-dihydro-2H-quinolin-1-yl]prop-2-en-1-one (3.9 mg, 7.8 umol, 8.0% yield, 100% purity, 0.1 FA) as a gray solid.

### Procedure for Preparation of Compound 160

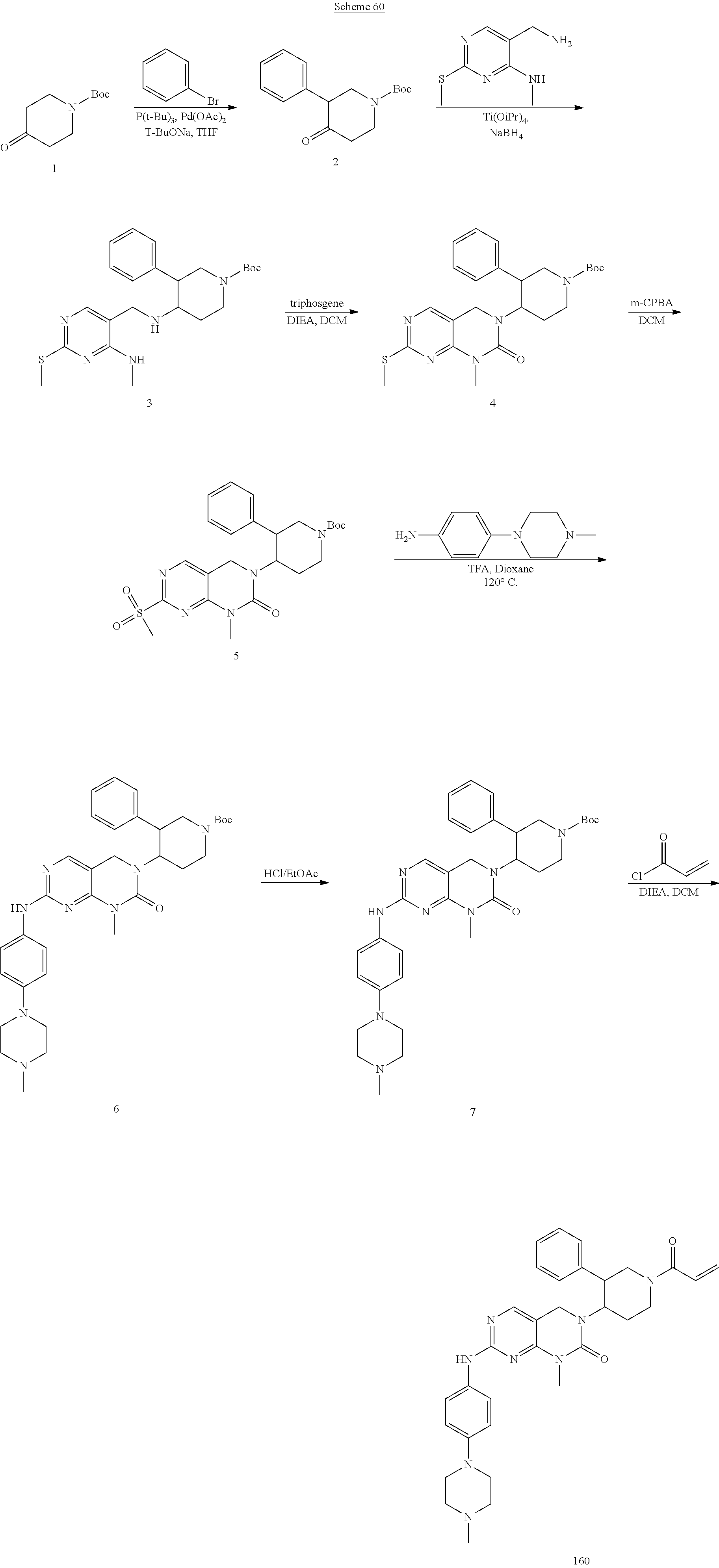

To a solution of tert-butyl 4-oxopiperidine-1-carboxylate (5 g, 25.09 mmol, 1 eq) and bromobenzene (7.88 g, 50.19 mmol, 5.29 mL, 2 eq) in THE (100 mL) was added t-BuONa (3.62 g, 37.64 mmol, 1.5 eq), Pd(OAc)₂ (281.70 mg, 1.25 mmol, 0.05 eq) and t-Bu₃P (330.01 mg, 1.63 mmol, 382.84 uL, 0.065 eq) at 20° C. under N₂. The reaction mixture was evacuated and recharged with N₂ for 3 times and then stirred at 65° C. for 12 hr under N₂. LCMS showed the desired product mass peak was detected. The mixture was concentrated under vacuum. The residue was diluted with 150 mL brine and extracted with EtOAc (300 mL*3). The combined organic phase was dried with anhydrous Na₂SO₄, the mixture was filtered and the filtrate was concentrated under vacuum. The residue was purified by flash silica gel chromatography (ISCO®; 80 g SepaFlash® Silica Flash Column, Eluent of 0-8% Ethyl acetate/Petroleum ether gradient @ 100 mL/min) to give tert-butyl 4-oxo-S-phenyl-piperidine-1-carboxylate (6 g, 21.79 mmol, 43.42% yield) as a yellow oil.

1H NMR (400 MHz, CHLOROFORM-d) δ ppm 7.29-7.40 (m, 3H) 7.19 (br d, J=7.13 Hz, 2 H) 3.93-4.41 (m, 2H) 3.44-3.81 (m, 3H) 2.39-2.77 (m, 2H) 1.42-1.68 (m, 9H)

To a solution of tert-butyl 4-oxo-S-phenyl-piperidine-1-carboxylate (2.5 g, 9.08 mmol, 1 eq) and 5-(aminomethyl)-N-methyl-2-methylsulfanyl-pyrimidin-4-amine (1.67 g, 9.08 mmol, 1 eq) in MeOH (20 mL) was added Ti(i-PrO)₄ (7.74 g, 27.24 mmol, 8.04 mL, 3 eq) at 20° C. under N₂. The mixture was stirred at 30° C. for 12 hr under N₂. Then NaBH₄ (2.06 g, 54.48 mmol, 6 eq) was added to the mixture at 0° C. The mixture was stirred at 20° C. for 12 hr.

LCMS showed the desired product mass peak was detected. The reaction mixture was quenched with NH₄Cl solution (30 mL) at 0° C., The reaction mixture was diluted with 50 mL MeOH. The mixture was filtered and the filtrate was concentrated under vacuum. The residue was diluted with 20 mL H₂O and extracted with EtOAc(50 mL*3). The combined organic phase was dried with anhydrous Na₂SO₄, the mixture was filtered and the filtrate was concentrated under vacuum. The residue was purified by flash silica gel chromatography (ISCO; 40 g SepaFlash Silica Flash Column, Eluent of 0-40% Ethyl acetate/Petroleum ether gradient @ 50 mL/min) to give tert-butyl 4-[[4-(methylamino)-2-methylsulfanyl-pyrimidin-5-yl]methylamino]-S-phenyl-piperidine-1-carboxylate (1.4 g, 3.16 mmol, 34.76% yield) as a yellow oil.

To a solution of tert-butyl 4-[[4-(methylamino)-2-methylsulfanyl-pyrimidin-5-yl]methylamino]-S-phenyl-piperidine-1-carboxylate (1.40 g, 3.16 mmol, 1 eq) in DCM(18 mL) was added DIPEA (1.22 g, 9.47 mmol, 1.65 mL, 3 eq) at 0° C. Then bis(trichloromethyl) carbonate (936.53 mg, 3.16 mmol, 1 eq) in DCM(2 mL) was added to the mixture at 0° C. The mixture was stirred at 20° C. for 12 hr. LCMS showed the reaction was completed and desired product mass peak was detected. The reaction mixture was quenched with saturated NaHCO₃ aqueous solution(30 mL) at 0° C., then the mixture was extracted with DCM (50 mL*3). The combined organic phase was dried with anhydrous Na₂SO₄, the mixture was filtered and the filtrate was concentrated under vacuum. The residue was purified by flash silica gel chromatography (ISCO®; 20 g SepaFlash® Silica Flash Column, Eluent of 0-35% Ethyl acetate/Petroleum ether gradient @ 20 mL/min) to give tert-butyl 4-(1-methyl-7-methylsulfanyl-2-oxo-4H-pyrimido[4,5-d]pyrimidin-S-yl)-S-phenyl-piperidine-1-carboxylate (900 mg, 1.92 mmol, 60.73% yield) as a yellow solid.

1H NMR (400 MHz, CHLOROFORM-d) δ ppm 7.45 (s, 1H) 7.27 (br s, 5H) 4.10-4.26 (m, 2H) 3.74-3.87 (m, 2H) 3.47 (br d, J=1.00 Hz, 3H) 2.51-2.66 (m, 3H) 2.04-2.20 (m, 4H) 1.53 (br s, 9H) 1.30-1.34 (m, 2H)

To a solution of tert-butyl 4-(1-methyl-7-methylsulfanyl-2-oxo-4H-pyrimido[4,5-d]pyrimidin-S-yl)-S-phenyl-piperidine-1-carboxylate (900.00 mg, 1.92 mmol, 1 eq) in DCM (10 mL) was added MCPBA (826.82 mg, 4.79 mmol, 2.5 eq) at 0° C. The mixture was stirred at 20° C. for 1.5 hr. LCMS showed the reaction was completed and desired product was detected. The reaction mixture was quenched with saturated Na₂SO₃ aqueous solution(15 mL) at 0° C., then the mixture was extracted with DCM (15 mL*3). The combined organic phase was dried with anhydrous Na₂SO₄, the mixture was filtered and the filtrate was concentrated under vacuum. The residue was purified by flash silica gel chromatography (ISCO®; 12 g SepaFlash® Silica Flash Column, Eluent of 0-45% Ethyl acetate/Petroleum ether gradient @ 15 mL/min) to give tert-butyl 4-(1-methyl-7-methylsulfonyl-2-oxo-4H-pyrimido[4,5-d]pyrimidin-S-yl)-S-phenyl-piperidine-1-carboxylate (550 mg, 1.10 mmol, 57.21% yield) as a white solid.

To a solution of tert-butyl 4-(1-methyl-7-methylsulfonyl-2-oxo-4H-pyrimido[4,5-d]pyrimidin-S-yl)-S-phenyl-piperidine-1-carboxylate (530 mg, 1.06 mmol, 1 eq) and 4-(4-methylpiperazin-1-yl)aniline (606.31 mg, 3.17 mmol, 3 eq) in dioxane (10 mL) was added TFA (240.95 mg, 2.11 mmol, 156.46 uL, 2 eq) at 20° C. under N₂. The mixture was stirred at 120° C. for 12 hr under the N₂. LCMS showed the desired product mass peak was detected.

The reaction mixture was concentrated under reduced pressure. The residue was purified by prep-HPLC (FA condition, column: Phenomenex Luna C18 75*30 mm*3 um;mobile phase: [water(FA)-ACN];B %: 20%-50%, 8 min) to give tert-butyl 4-[1-methyl-7-[4-(4-methylpiperazin-1-yl)anilino]-2-oxo-4H-pyrimido[4,5-d]pyrimidin-S-yl]-S-phenyl-piperidine-1-carboxylate (140 mg, 194.62 umol, 18.42% yield, 91.58% purity, FA) as a white solid.

1H NMR (400 MHz, CHLOROFORM-d) δ ppm 8.40 (br s, 1H) 7.46 (d, J=8.76 Hz, 2H) 7.29 (s, 1H) 7.15-7.26 (m, 5H) 6.89-7.01 (m, 3H) 4.83 (dt, J=13.20, 4.22 Hz, 1H) 4.28-4.66 (m, 2H) 3.72 (d, J=13.63 Hz, 1H) 3.41 (s, 3H) 3.28-3.38 (m, 1H) 3.16-3.27 (m, 5H) 2.92-3.02 (m, 1H) 2.70-2.79 (m, 5H) 2.44 (s, 3H) 2.07 (qd, J=12.44, 4.19 Hz, 1H) 1.63 (br d, J=11.38 Hz, 1H) 1.43-1.52 (m, 9H)

To a solution of tert-butyl 4-[1-methyl-7-[4-(4-methylpiperazin-1-yl)anilino]-2-oxo-4H-pyrimido[4,5-d]pyrimidin-S-yl]-S-phenyl-piperidine-1-carboxylate (120.00 mg, 195.83 umol, 1 eq) in HCl/EtOAc (4 mL). The mixture was stirred at 20° C. for 1 hr. LCMS showed the reaction was completed and desired product was detected. The reaction mixture was concentrated under reduced pressure to give 1-methyl-7-[4-(4-methylpiperazin-1-yl)anilino]-3-(3-phenyl-4-piperidyl)-4H-pyrimido[4,5-d]pyrimidin-2-one (100 mg, crude, HCl) as a white solid.

To a solution of 1-methyl-7-[4-(4-methylpiperazin-1-yl)anilino]-S-(3-phenyl-4-piperidyl)-4H-pyrimido[4,5-d]pyrimidin-2-one (100.00 mg, 182.11 umol, 1 eq, HCl) in DCM(2 ml) was added DIPEA (70.61 mg, 546.34 umol, 95.16 uL, 3 eq) The prop-2-enoyl chloride (16.48 mg, 182.11 umol, 14.85 uL, 1 eq) in 6CM (0.2 ml) was added to the mixture under N₂ atmosphere at 0° C. The mixture was stirred at 0° C. for 1 hr under N₂. LCMS showed detection of the desired product mass peak. The reaction mixture was concentrated under reduced pressure. The residue was purified by prep-HPLC (FA condition; column: Phenomenex Luna C18 75*30 mm*3 um;mobile phase: [water(FA)-ACN];B %: 1%-30%, 8 min) to give 1-methyl-7-[4-(4-methylpiperazin-1-yl)anilino]-S-(3-phenyl-1-prop-2-enoyl-4-piperidyl)-4H-pyrimido[4,5-d]pyrimidin-2-one (22.6 mg, 36.88 umol, 20.25% yield, 100% purity, FA) as a white solid.

TABLE 3

| Cmpd | | LCMS | |
|---|---|---|---|
| No. | Structure | (M + H) | ¹H NMR (DMSO) |
| 117 | 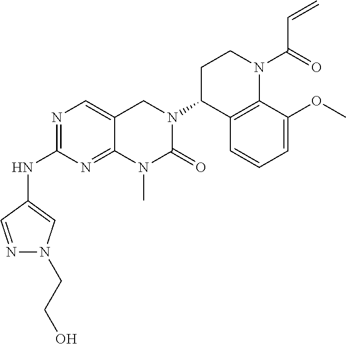 [SMILES: C=CC(=O)N1CC[C@@H](N2Cc3cnc(Nc4cnn(CCO)c4)nc3N(C)C2=O)c2cccc(OC)c21] | 505.2 | δ = 9.00-9.08 (m, 1 H) 7.91- 7.98 (m, 1 H) 7.84-7.88 (m, 1 H) 7.49-7.56 (m, 1 H) 7.17- 7.24 (m, 1 H) 6.99-7.05 (m, 1 H) 6.80 (br d, J = 7.75 Hz, 1 H) 6.19-6.35 (m, 1 H) 6.06-6.15 (m, 1 H) 5.41-5.65 (m, 2 H) 3.83-4.75 (m, 6 H) 3.69-3.79 (m, 5 H) 3.35 (s, 3 H) 1.96- 2.30 (m, 2 H) |
| 118 | 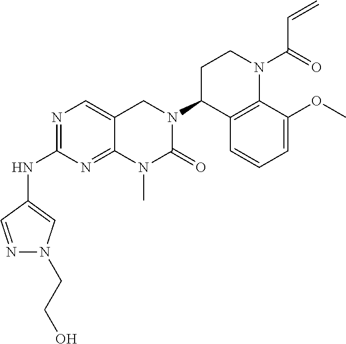 [SMILES: C=CC(=O)N1CC[C@H](N2Cc3cnc(Nc4cnn(CCO)c4)nc3N(C)C2=O)c2cccc(OC)c21] | 505.2 | δ = 9.50-9.21 (m, 1H), 8.08- 7.79 (m, 2H), 7.51 (br s, 1H), 7.21 (br d, J = 0.8 Hz, 1H), 7.03 (br s, 1H), 6.89-6.68 (m, 1H), 6.37-6.07 (m, 2H), 5.76-5.41 (m, 2H), 4.92-4.02 (m, 6H), 3.73 (s, 3H), 3.70 (br s, 2H), 3.31-3.07 (m, 3H), 2.34-1.86 (m, 2H) |
| 119 | 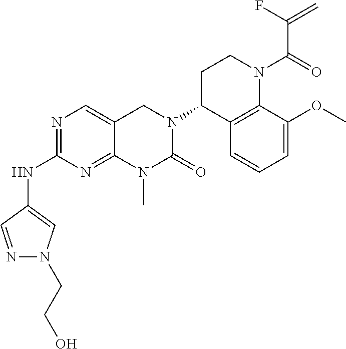 [SMILES: C=C(F)C(=O)N1CC[C@@H](N2Cc3cnc(Nc4cnn(CCO)c4)nc3N(C)C2=O)c2cccc(OC)c21] | 523.2 | δ = 9.20-9.46 (m, 1 H) 7.88- 7.98 (m, 2 H) 7.53 (s, 1 H) 6.65- 6.76 (m, 1 H) 6.44-6.51 (m, 2 H) 5.54-5.80 (m, 3 H) 5.15- 5.23 (m, 1 H) 4.48-4.54 (m, 2 H) 4.37-4.44 (m, 2 H) 4.03- 4.16 (m, 1 H) 3.80-3.90 (m, 1 H) 3.76 (s, 3 H) 3.21-3.32 (m, 5 H) 1.99-2.14 (m, 1 H) 1.83- 1.97 (m, 1 H) |
| 120 | 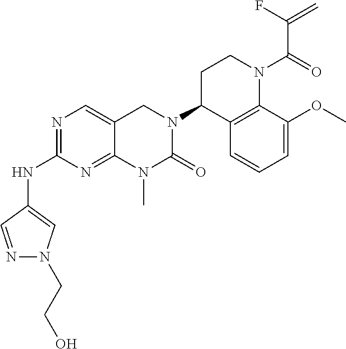 [SMILES: C=C(F)C(=O)N1CC[C@H](N2Cc3cnc(Nc4cnn(CCO)c4)nc3N(C)C2=O)c2cccc(OC)c21] | 523.2 | δ = 9.36 (br s, 1H), 8.00-7.88 (m, 2H), 7.53 (s, 1H), 6.75- 6.64 (m, 1H), 6.47 (br d, J = 3.5 Hz, 2H), 5.79-5.55 (m, 3H), 5.19 (br s, 1H), 4.50 (br d, J = 4.5 Hz, 2H), 4.41 (br d, J = 3.8 Hz, 2H), 4.10 (br d, J = 14.3 Hz, 1H), 3.84 (br d, J = 14.3 Hz, 1H), 3.76 (s, 3H), 3.30-3.24 (m, 3H), 2.07-2.01 (m, 1H), 1.94-1.87 (m, 1H) |
| 121 | 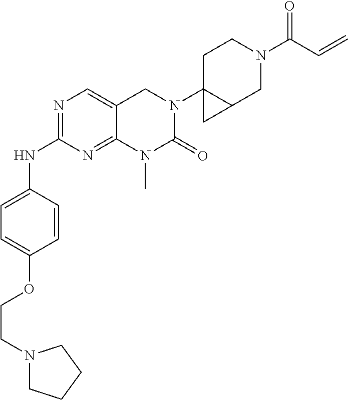 [SMILES: C=CC(=O)N1CCC2(N3Cc4cnc(Nc5ccc(OCCN6CCCC6)cc5)nc4N(C)C3=O)CC2C1] | 518.3 | δ = 9.23-9.39 (m, 1 H) 8.21 (s, 1 H) 7.99-8.06 (m, 1 H) 7.61 (d, J = 9.01 Hz, 2 H) 6.73- 7.05 (m, 3 H) 6.06-6.27 (m, 1 H) 5.63-5.81 (m, 1 H) 4.88- 5.04 (m, 1 H) 4.23-4.40 (m, 3 H) 4.04 (t, J = 5.82 Hz, 2 H) 3.28 (s, 3 H) 3.03-3.15 (m, 1 H) 2.86 (t, J = 5.82 Hz, 2 H) 2.59- 2.68 (m, 5 H) 1.67-1.76 (m, 4 H) 1.49-1.64 (m, 2 H) 1.32- 1.44 (m, 1 H) 1.14 (dt, J = 9.29, 5.74 Hz, 1 H) 0.87-1.05 (m, 1 H) |
| 122 | 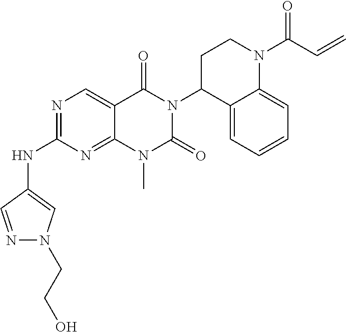 [SMILES: C=CC(=O)N1CCC(n2c(=O)c3cnc(Nc4cnn(CCO)c4)nc3n(C)c2=O)c2ccccc21] | 489.1 | δ = 10.46 (s, 1H), 8.78 (br s, 1H), 8.00-7.95 (m, 1H), 7.68- 7.55 (m, 1H), 7.23-7.17 (m, 1H), 7.15-7.06 (m, 3H), 6.60 (br dd, J = 10.3, 16.5 Hz, 1H), 6.30-6.14 (m, 2H), 5.75 (br d, J = 10.4 Hz, 1H), 4.90 (br s, 1H), 4.46-4.35 (m, 1H), 4.18- 4.07 (m, 2H), 3.72 (br d, J = 4.5 Hz, 2H), 3.61 (br t, J = 10.9 Hz, 1H), 3.49 (br s, 3H), 2.33 (br s, 1H), 2.24-2.13 (m, 1H) |
| 123 | 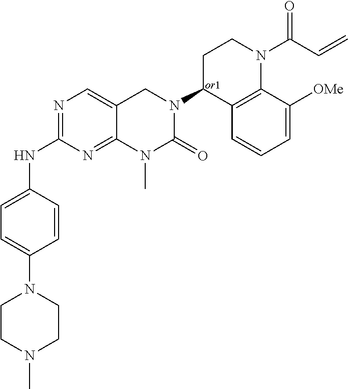 [SMILES: C=CC(=O)N1CC[C@H](N2Cc3cnc(Nc4ccc(N5CCN(C)CC5)cc4)nc3N(C)C2=O)c2cccc(OC)c21] | 569.2 | 298K: δ = 9.24 (br d, J = 14.5 Hz, 1 H) 8.18 (s, 1 H) 7.88- 8.05 (m, 1 H) 7.55 (br s, 2 H) 7.21 (br s, 1 H) 6.97-7.09 (m, 1 H) 6.70-6.91 (m, 3 H) 6.07- 6.40 (m, 2 H) 5.42-5.79 (m, 2 H) 3.85-4.69 (m, 4 H) 3.72- 3.76 (m, 3 H) 3.30 (br s, 3 H) 2.98-3.09 (m, 4 H) 2.38-2.48 (m, 4 H) 1.81-2.29 (m, 5 H) 273 + 80K: δ = 8.86-8.95 (m, 1 H) 8.06-8.20 (m, 1 H) 7.88- 7.99 (m, 1 H) 7.49-7.58 (m, 2 H) 7.15-7.26 (m, 1 H) 7.01 (d, J = 8.13 Hz, 1 H) 6.77-6.89 (m, 3 H) 6.03-6.35 (m, 2 H) 5.40- 5.68 (m, 2 H) 3.90-4.26 (m, 2 H) 3.70-3.76 (m, 3 H) 3.32 (s, 5 H) 3.06-3.09 (m, 4 H) 2.44- 2.48 (m, 4 H) 1.95-2.25 (m, 5 H) |
| 124 | 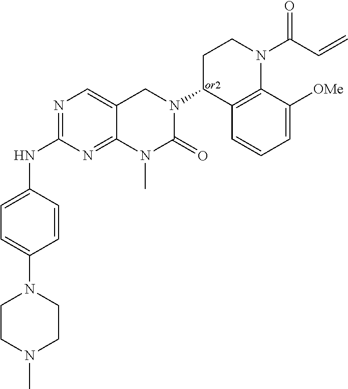 [SMILES: C=CC(=O)N1CC[C@@H](N2Cc3cnc(Nc4ccc(N5CCN(C)CC5)cc4)nc3N(C)C2=O)c2cccc(OC)c21] | 569.3 | 298K: δ = 9.24 (br d, J = 14.1 Hz, 1H), 8.27 (s, 1H), 8.09- 7.82 (m, 1H), 7.55 (br s, 2H), 7.21 (br s, 1H), 7.08-6.94 (m, 1H), 6.87 (br d, J = 8.4 Hz, 2H), 6.84-6.71 (m, 1H), 6.36-6.09 (m, 2H), 5.78-5.43 (m, 2H), 4.69-4.40 (m, 1H), 4.27 (br t, J = 6.1 Hz, 1H), 4.13 (br d, J = 12.9 Hz, 1H), 4.07-3.95 (m, 1H), 3.82 (br d, J = 13.3 Hz, 1H), 3.74 (s, 3H), 3.31 (br s, 3H), 3.05 (br s, 4H), 2.44 (br s, 4H), 2.21 (s, 3H), 2.16-1.87 (m, 2H) 273 + 80K: δ = 8.92 (s, 1H), 8.24 (s, 1H), 7.94 (s, 1H), 7.66-7.49 (m, 2H), 7.25-7.15 (m, 1H), 7.06-6.94 (m, 1H), 6.88-6.84 (m, 2H), 6.80 (br d, J = 7.6 Hz, 1H), 6.34-6.20 (m, 1H), 6.16-6.08 (m, 1H), 6.11- 6.08 (m, 1H), 5.57 (dd, J = 2.4, 10.2 Hz, 2H), 4.31 (br t, J = 6.3 Hz, 1H), 4.19 (br s, 1H), 4.04- 3.92 (m, 1H), 3.75 (s, 3H), 3.74- 3.65 (m, 1H), 3.59-3.48 (m, 1H), 3.33 (s, 3H), 3.09-3.06 (m, 4H), 2.47 (br d, J = 5.0 Hz, 4H), 2.24 (s, 3H), 2.19-1.96 (m, 2H) |
| 125 | 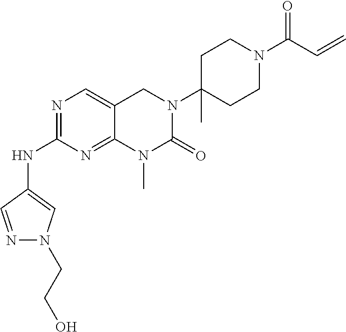 [SMILES: C=CC(=O)N1CCC(C)(N2Cc3cnc(Nc4cnn(CCO)c4)nc3N(C)C2=O)CC1] | 441.2 | δ = 9.41 (br d, J = 9.4 Hz, 1H), 8.08 (s, 1H), 7.88 (s, 1H), 7.50 (s, 1H), 6.77 (dd, J = 10.5, 16.8 Hz, 1H), 6.05 (dd, J = 2.4, 16.7 Hz, 1H), 5.70-5.59 (m, 1H), 4.87 (t, J = 5.3 Hz, 1H), 4.27- 4.12 (m, 2H), 4.08 (t, J = 5.6 Hz, 2H), 3.83-3.74 (m, 1H), 3.70 (q, J = 5.5 Hz, 2H), 3.66- 3.58 (m, 1H), 3.26 (br s, 4H), 3.18-3.06 (m, 1H), 2.49-2.43 (m, 1H), 2.42-2.34 (m, 1H), 1.77-1.60 (m, 2H), 1.39 (s, 3H) |
| 126 | 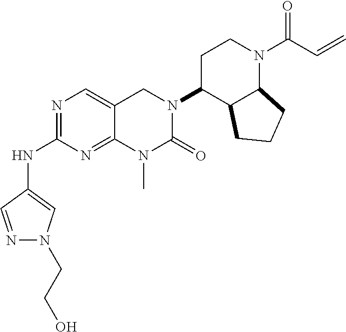 | 467.2 | δ = 9.53-9.63 (m, 1 H) 8.02- 8.10 (m, 1 H) 7.54-7.60 (m, 1 H) 6.64-6.77 (m, 1 H) 6.56 (d, J = 2.25 Hz, 1 H) 6.03 (dd, J = 16.76, 2.38 Hz, 1 H) 5.58- 5.72 (m, 1 H) 4.86 (t, J = 5.32 Hz, 1 H) 4.16-4.30 (m, 3 H) 3.95-4.06 (m, 3 H) 3.71 (q, J = 5.59 Hz, 2 H) 3.18-3.28 (m, 4 H) 3.06 (td, J = 10.91, 6.19 Hz, 1 H) 1.46-2.03 (m, 8 H) 1.09- 1.24 (m, 1 H) |
| 127 | 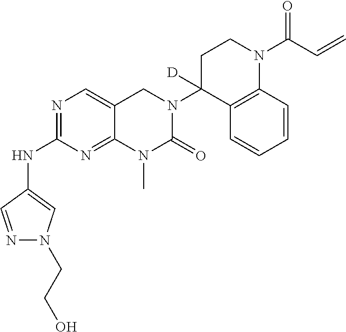 [SMILES: [2H]C1(N2Cc3cnc(Nc4cnn(CCO)c4)nc3N(C)C2=O)CCN(C(=O)C=C)c2ccccc21] | 476.2 | δ = 9.34 (br s, 1H), 7.98 (s, 1H), 7.89 (s, 1H), 7.52 (s, 1H), 7.32-7.21 (m, 2H), 7.19 (d, J = 4.5 Hz, 2H), 6.65 (dd, J = 10.3, 16.8 Hz, 1H), 6.31-6.23 (m, 1H), 5.82-5.76 (m, 1H), 4.88 (t, J = 5.3 Hz, 1H), 4.26-4.13 (m, 2H), 4.09 (t, J = 5.5 Hz, 2H), 3.90 (d, J = 14.5 Hz, 1H), 3.77-3.67 (m, 3H), 3.33-3.33 (m, 3H), 2.26-2.14 (m, 1H), 2.11-1.90 (m, 1H) |
| 128 | 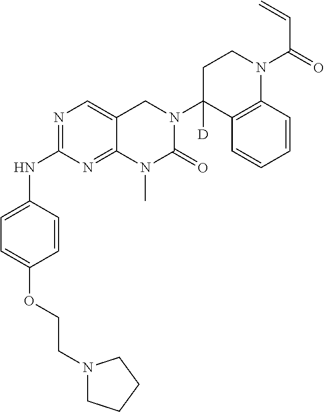 [SMILES: [2H]C1(N2Cc3cnc(Nc4ccc(OCCN5CCCC5)cc4)nc3N(C)C2=O)CCN(C(=O)C=C)c2ccccc21] | 555.3 | δ = 9.30 (s, 1H), 7.99 (s, 1H), 7.66-7.56 (m, 2H), 7.33-7.23 (m, 2H), 7.19 (d, J = 4.5 Hz, 2H), 6.86 (d, J = 9.0 Hz, 2H), 6.73-6.52 (m, 1H), 6.26 (dd, J = 2.3, 16.8 Hz, 1H), 5.86-5.68 (m, 1H), 4.23 (d, J = 14.5 Hz, 1H), 4.17 (br d, J = 13.0 Hz, 1H), 4.01 (t, J = 6.0 Hz, 2H), 3.90 (d, J = 14.0 Hz, 1H), 3.71 (br s, 1H), 3.33 (s, 3H), 2.76 (br t, J = 5.8 Hz, 2H), 2.54-2.51 (m, 4H), 2.18 (s, 1H), 2.10- 1.98 (m, 1H), 1.68 (td, J = 3.3, 6.5 Hz, 4H) |
| 129 | 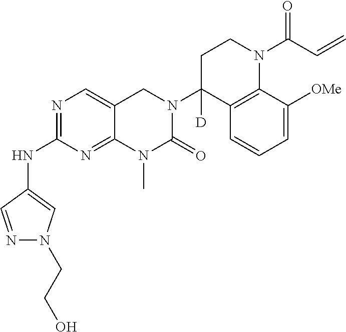 [SMILES: [2H]C1(N2Cc3cnc(Nc4cnn(CCO)c4)nc3N(C)C2=O)CCN(C(=O)C=C)c2c(OC)cccc21] | 506.2 | δ = 9.16-9.30 (m, 1 H) 8.00- 8.10 (m, 1 H) 7.56 (d, J = 9.01 Hz, 2 H) 7.22-7.42 (m, 3 H) 7.13-7.21 (m, 1 H) 6.93 (d, J = 9.01 Hz, 2 H) 6.70 (dd, J = 16.70, 10.32 Hz, 1 H) 6.32 (dd, J = 16.82, 2.06 Hz, 1 H) 5.79-5.90 (m, 1 H) 5.63-5.73 (m, 1 H) 5.03-5.35 (m, 1 H) 4.13-4.29 (m, 2 H) 3.86-3.97 (m, 1 H) 3.73-3.84 (m, 1 H) 3.00-3.20 (m, 5 H) 2.58 (br d, J = 2.00 Hz, 4 H) 2.29-2.36 (m, 3 H) 2.18-2.28 (m, 3 H) 2.09 (ddt, J = 18.29, 10.13, 5.05, 5.05 Hz, 1 H) 1.84-1.99 (m, 4 H) 1.54-1.67 (m, 2 H) |
| 130 | 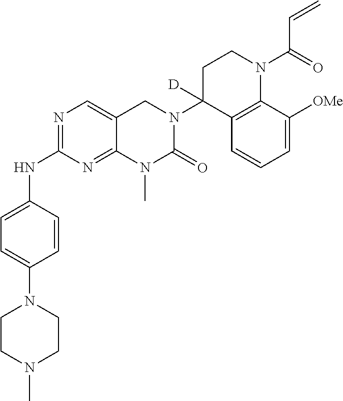 [SMILES: [2H]C1(N2Cc3cnc(Nc4ccc(N5CCN(C)CC5)cc4)nc3N(C)C2=O)CCN(C(=O)C=C)c2c(OC)cccc21] | 570.3 | δ = 9.25 (br d, J = 14.43 Hz, 1 H) 7.87-8.10 (m, 1 H) 7.56 (br s, 2 H) 7.13-7.28 (m, 1 H) 6.98- 7.08 (m, 1 H) 6.68-6.92 (m, 3 H) 6.09-6.37 (m, 2 H) 5.50- 5.68 (m, 1 H) 3.78-4.75 (m, 4 H) 3.75 (s, 3 H) 3.06 (br s, 4 H) 2.40-2.47 (m, 5 H) 1.86-2.32 (m, 5 H) |
| 131 | 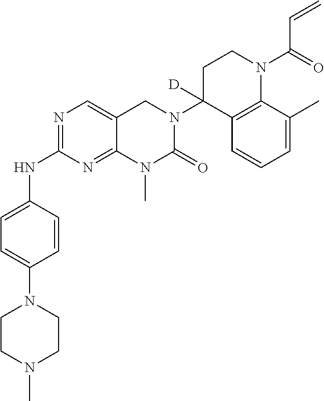 [SMILES: [2H]C1(N2Cc3cnc(Nc4ccc(N5CCN(C)CC5)cc4)nc3N(C)C2=O)CCN(C(=O)C=C)c2c(C)cccc21] | 554.4 | 298K: δ = 9.37-9.08 (m, 1H), 8.17-7.83 (m, 1H), 7.55 (br dd, J = 8.9, 13.1 Hz, 2H), 7.29- 6.91 (m, 3H), 6.87 (br dd, J = 5.6, 8.3 Hz, 2H), 6.39-5.93 (m, 2H), 5.87-5.61 (m, 1H), 4.79- 4.56 (m, 1H), 4.43-4.21 (m, 1H), 4.15-3.96 (m, 1H), 3.87- 3.62 (m, 1H), 3.31 (br s, 3H), 3.04 (br s, 4H), 2.44 (br s, 4H), 2.21 (s, 3H), 2.18-1.81 (m, 5H) 273 + 80K: δ = 8.90 (s, 1H), 7.92 (dt, J = 2.6, 3.6 Hz, 1H), 7.54 (br d, J = 8.9 Hz, 2H), 7.18 (q, J = 7.3 Hz, 2H), 7.04 (br s, 1H), 6.86 (d, J = 9.0 Hz, 2H), 6.30-6.20 (m, 1H), 5.81-5.63 (m, 1H), 4.34-3.78 (m, 3H), 3.40-3.20 (m, 4H), 3.06 (br s, 4H), 2.48-2.45 (m, 5H), 2.23 (s, 3H), 2.13 (s, 3H), 2.07-1.92 (m, 1H) |
| 132 | 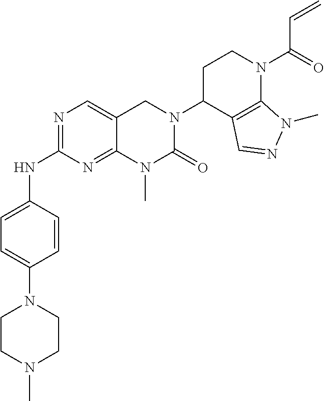 [SMILES: C=CC(=O)N1CCC(N2Cc3cnc(Nc4ccc(N5CCN(C)CC5)cc4)nc3N(C)C2=O)c2cnn(C)c21] | 543.3 | δ = 9.27-9.09 (m, 1H), 8.29- 8.21 (m, 1H), 7.97 (s, 0.3H), 7.62-7.47 (m, 2H), 7.38-7.19 (m, 1H), 6.86 (d, J = 9.2 Hz, 2H), 6.43-6.26 (m, 1H), 6.04- 5.84 (m, 1H), 5.70-5.56 (m, 1H), 4.19-4.02 (m, 2H), 3.96- 3.91 (m, 1H), 3.81-3.72 (m, 1H), 3.59 (s, 3H), 3.30 (s, 3H), 3.08-2.98 (m, 4H), 2.47-2.39 (m, 4H), 2.21 (s, 3H), 2.12- 2.00 (m, 1H), 1.94-1.79 (m, 1H |
| 133 | 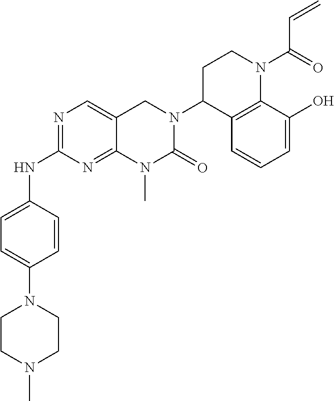 [SMILES: C=CC(=O)N1CCC(N2Cc3cnc(Nc4ccc(N5CCN(C)CC5)cc4)nc3N(C)C2=O)c2cccc(O)c21] | 555.3 | 298K: δ = 10.40-9.66 (m, 1H), 9.32-9.15 (m, 1H), 8.23 (s, 1H), 7.96 (br d, J = 8.5 Hz, 1H), 7.55 (br d, J = 8.8 Hz, 2H), 7.05 (t, J = 7.8 Hz, 1H), 6.92-6.76 (m, 3H), 6.63 (br s, 1H), 6.50- 6.24 (m, 1H), 6.24-6.06 (m, 1H), 5.84-5.11 (m, 2H), 4.75- 3.66 (m, 2H), 3.32-3.29 (m, 5H), 3.07-3.02 (m, 4H), 2.47- 2.42 (m, 4H), 2.21 (s, 3H), 1.95 (br dd, J = 10.2, 12.4 Hz, 2H) 273 + 80K: δ = 8.90 (s, 1H), 8.21 (s, 1H), 7.93 (s, 1H), 7.54 (d, J = 8.9 Hz, 2H), 7.12-6.99 (m, 1H), 6.90-6.80 (m, 3H), 6.66 (d, J = 7.7 Hz, 1H), 6.53- 6.34 (m, 1H), 6.16 (dd, J = 2.2, 16.7 Hz, 1H), 5.67-5.46 (m, 2H), 4.25-3.68 (m, 4H), 3.34- 3.31 (m, 3H), 3.06 (br s, 4H), 2.48-2.45 (m, 4H), 2.30-2.19 (m, 4H), 2.09-1.95 (m, 1H) |
| 134 | 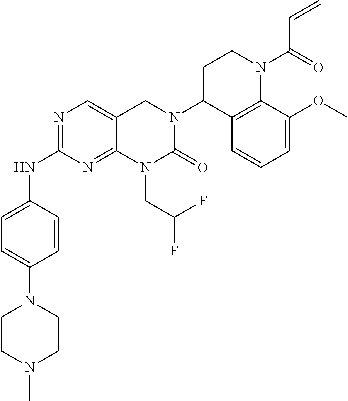 [SMILES: C=CC(=O)N1CCC(N2Cc3cnc(Nc4ccc(N5CCN(C)CC5)cc4)nc3N(CC(F)F)C2=O)c2cccc(OC)c21] | 619.4 | δ = 9.71-9.56 (m, 1H), 9.50- 9.31 (m, 1H), 8.14-7.98 (m, 1H), 7.56 (br s, 2H), 7.28-7.17 (m, 1H), 7.04 (br d, J = 7.6 Hz, 1H), 6.94 (br d, J = 8.8 Hz, 2H), 6.85-6.65 (m, 1H), 6.54-6.42 (m, 1H), 6.37-6.26 (m, 1H), 6.22-6.09 (m, 2H), 5.88-5.38 (m, 2H), 4.71-4.44 (m, 2H), 4.38-4.15 (m, 2H), 3.74 (s, 3H), 3.64 (br d, J = 5.1 Hz, 4H), 3.21-2.89 (m, 4H), 2.86 (br d, J = 3.6 Hz, 3H), 2.32-1.74 (m, 2H) |
| 135 | 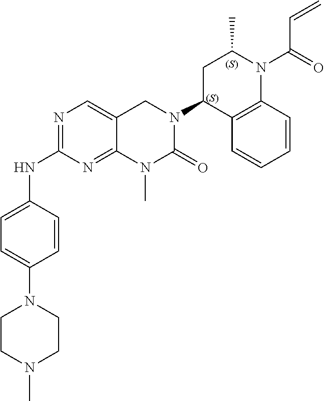 | 553.3 | δ = 9.25 (s, 1H), 7.95 (s, 1H), 7.61-7.52 (m, 2H), 7.36-7.04 (m, 4H), 6.89 (br d, J = 9.0 Hz, 2H), 6.63 (dd, J = 10.4, 16.8 Hz, 1H), 6.26 (dd, J = 2.0, 16.8 Hz, 1H), 5.82-5.66 (m, 2H), 5.00 (br s, 1H), 4.16 (d, J = 14.1 Hz, 1H), 3.83 (d, J = 14.2 Hz, 1H), 3.32 (br s, 3H), 3.12 (br s, 4H), 2.83-2.58 (m, 4H), 2.42 (br s, 3H), 2.29-2.17 (m, 1H), 1.96 (ddd, J = 2.7, 7.4, 13.4 Hz, 1H), 1.16 (d, J = 6.8 Hz, 3H) |
| 136 | 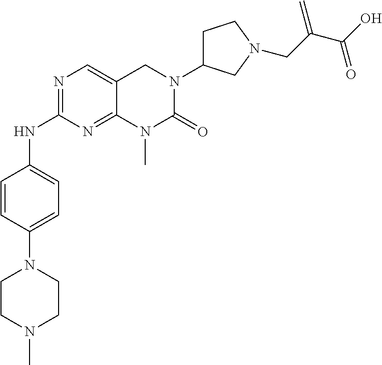 [SMILES: C=C(CN1CCC(N2Cc3cnc(Nc4ccc(N5CCN(C)CC5)cc4)nc3N(C)C2=O)C1)C(=O)O] | 507.4 | δ = 9.21 (s, 1H), 8.17 (s, 1H), 8.06 (s, 1H), 7.55 (d, J = 9.1 Hz, 2H), 6.87 (d, J = 9.1 Hz, 2H), 6.10 (s, 1H), 5.73 (s, 1H), 5.00-4.87 (m, 1H), 4.31 (d, J = 2.8 Hz, 2H), 3.34 (br s, 1H), 3.31 (s, 1H), 3.26 (s, 1H), 3.24 (s, 3H), 3.07-3.02 (m, 4H), 2.88 (dt, J = 4.0, 8.7 Hz, 1H), 2.78 (dd, J = 4.1, 10.3 Hz, 1H), 2.56-2.52 (m, 1H), 2.48-2.45 (m, 3H), 2.36 (q, J = 8.3 Hz, 1H), 2.23 (s, 3H), 2.14-2.00 (m, 1H), 1.92-1.78 (m, 1H) |
| 137 | 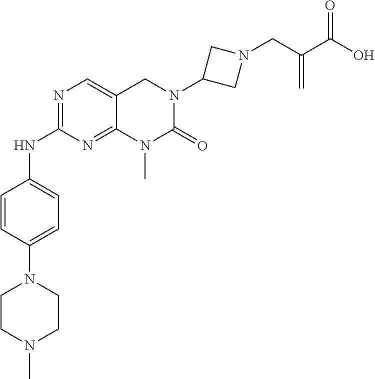 [SMILES: C=C(CN1CC(N2Cc3cnc(Nc4ccc(N5CCN(C)CC5)cc4)nc3N(C)C2=O)C1)C(=O)O] | 493.4 | δ = 9.22 (s, 1 H) 8.19 (s, 1 H) 8.09 (s, 1 H) 7.55 (d, J = 9.01 Hz, 2 H) 6.87 (d, J = 9.01 Hz, 2 H) 6.07 (s, 1 H) 5.66 (d, J = 1.13 Hz, 1 H) 4.60 (br t, J = 7.13 Hz, 1 H) 4.33 (s, 2 H) 3.54-3.60 (m, 5 H) 3.29 (s, 2 H) 3.18-3.26 (m, 5 H) 3.17 (s, 1 H) 3.02- 3.08 (m, 4 H) 2.24 (s, 3 H) |
| 138 | 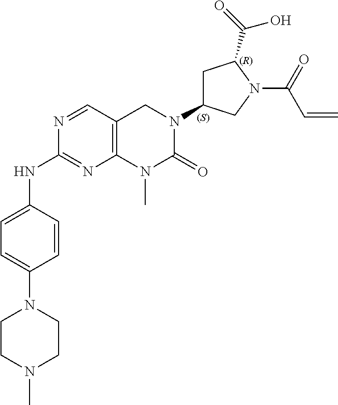 | 521.4 | δ = 9.25-9.19 (m, 1H), 8.18 (s, 1H), 8.04 (s, 1H), 7.55 (d, J = 9.0 Hz, 2H), 6.87 (d, J = 9.0 Hz, 2H), 6.68-6.39 (m, 1H), 6.23-6.06 (m, 1H), 5.76-5.59 (m, 1H), 5.13-4.87 (m, 1H), 4.69-4.47 (m, 1H), 4.33-4.23 (m, 2H), 3.90-3.81 (m, 1H), 3.76-3.66 (m, 1H), 3.25 (d, J = 2.0 Hz, 4H), 3.08-3.02 (m, 4H), 2.47 (br s, 3H), 2.23 (s, 3H), 2.10-2.02 (m, 1H) |
| 139 | 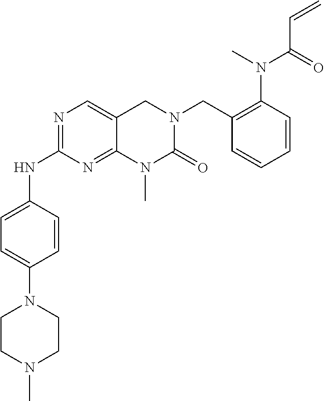 [SMILES: C=CC(=O)N(C)c1ccccc1CN1Cc2cnc(Nc3ccc(N4CCN(C)CC4)cc3)nc2N(C)C1=O] | 593.2 | δ = 9.22 (s, 1 H) 7.98 (s, 1 H) 7.50-7.61 (m, 2 H) 7.36-7.47 (m, 3 H) 7.20-7.33 (m, 1 H) 6.88 (d, J=9.16 Hz, 2 H) 6.05- 6.18 (m, 1 H) 5.83 (dd, J=16.75, 10.23 Hz, 1 H) 5.35- 5.55 (m, 1 H) 4.53-4.62 (m, 1 H) 4.35-4.44 (m, 1 H) 4.11- 4.32 (m, 2 H) 3.28 (s, 3 H) 3.19 (s, 3 H) 3.01-3.08 (m, 4 H) 2.41-2.47 (m, 4 H) 2.22 (s, 3 H) |
| 140 | 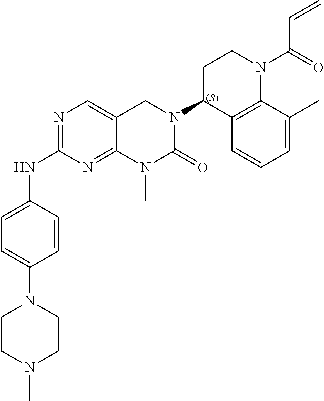 | 553.4 | δ = 9.06-8.72 (m, 1H), 8.08- 7.86 (m, 1H), 7.55 (br d, J = 8.7 Hz, 2H), 7.27-7.14 (m, 2H), 7.12-6.97 (m, 1H), 6.86 (d, J = 8.9 Hz, 2H), 6.33-6.20 (m, 1H), 5.70 (br s, 1H), 4.51-3.51 (m, 3H), 3.40-3.22 (m, 4H), 3.08 (br s, 4H), 2.49-2.44 (m, 5H), 2.24 (s, 4H), 2.14 (s, 3H), 2.10-1.91 (m, 1H) |
| 141 | 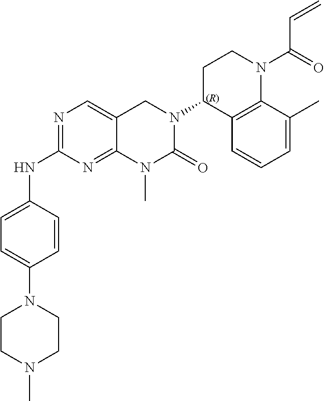 | 553.4 | 298K: δ = 9.35-9.15 (m, 1H), 8.26-7.81 (m, 1H), 7.62-7.47 (m, 2H), 7.31-6.94 (m, 3H), 6.87 (br dd, J = 5.5, 8.3 Hz, 2H), 6.37-6.18 (m, 1H), 6.07- 5.79 (m, 1H), 5.77-5.56 (m, 1H), 4.78-4.54 (m, 1H), 4.43- 4.20 (m, 1H), 4.14-3.96 (m, 1H), 3.88-3.52 (m, 1H), 3.11- 3.01 (m, 5H), 2.49-2.42 (m, 4H), 2.24 (s, 3H), 2.17-2.01 (m, 4H) 273 + 80K: δ = 8.92 (s, 1H), 8.05-7.84 (m, 1H), 7.55 (br d, J = 8.9 Hz, 2H), 7.23-7.15 (m, 2H), 7.11-6.98 (m, 1H), 6.88 (d, J = 9.1 Hz, 2H), 6.27 (br d, J = 16.1 Hz, 1H), 5.78-5.67 (m, 1H), 4.42-3.76 (m, 2H), 3.33 (s, 4H), 2.99 (br s, 4H), 2.50- 2.48 (m, 5H), 2.27 (s, 4H), 2.14 (s, 3H), 2.09-2.00 (m, 1H) |
| 142 | 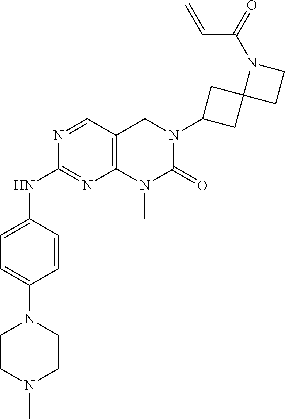 [SMILES: C=CC(=O)N1CCC12CC(N1Cc3cnc(Nc4ccc(N5CCN(C)CC5)cc4)nc3N(C)C1=O)C2] | 503.4 | δ = 9.20 (s, 1H), 8.12-8.07 (m, 1H), 7.55 (d, J = 9.0 Hz, 2H), 6.91-6.82 (m, 2H), 6.29- 6.09 (m, 2H), 5.71-5.63 (m, 1H), 4.58-4.45 (m, 1H), 4.39- 4.27 (m, 2H), 4.10 (t, J = 7.5 Hz, 2H), 3.26-3.17 (m, 5H), 3.09-3.01 (m, 4H), 2.46-2.36 (m, 6H), 2.30 (dt, J = 2.9, 8.6 Hz, 2H), 2.21 (s, 3H) |
| 143 | 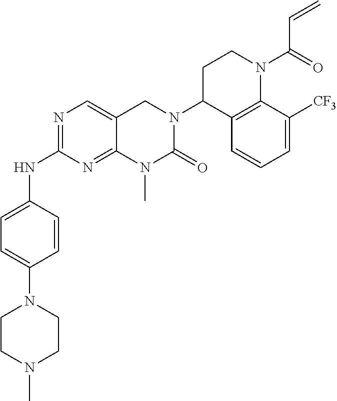 [SMILES: C=CC(=O)N1CCC(N2Cc3cnc(Nc4ccc(N5CCN(C)CC5)cc4)nc3N(C)C2=O)c2cccc(C(F)(F)F)c21] | 607.4 | δ = 8.23 (s, 1H), 7.30 (d, J = 8.0 Hz, 1H), 7.12 (d, J = 7.5 Hz, 1H), 7.05-6.99 (m, 2H), 6.97- 6.90 (m, 2H), 6.59 (t, J = 7.5 Hz, 1H), 6.55-6.40 (m, 1H), 6.22 (dd, J = 1.8, 16.8 Hz, 1H), 5.89 (br s, 1H), 5.76-5.68 (m, 1H), 5.63 (dd, J = 5.3, 10.3 Hz, 1H), 4.29 (d, J = 15.5 Hz, 1H), 4.01 (d, J = 15.5 Hz, 1H), 3.21 (s, 4H), 3.17-3.08 (m, 5H), 2.46-2.43 (m, 4H), 2.22 (s, 3H), 2.11-2.03 (m, 1H), 1.96- 1.89 (m, 1H) |
| 144 | 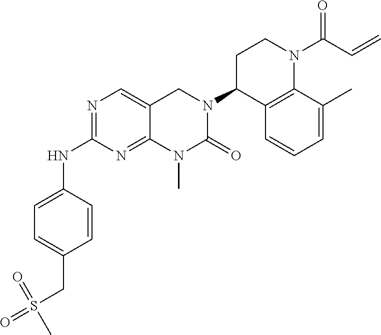 [SMILES: C=CC(=O)N1CC[C@H](N2Cc3cnc(Nc4ccc(CS(C)(=O)=O)cc4)nc3N(C)C2=O)c2cccc(C)c21] | 547.4 | δ = 9.38 (br s, 1H), 8.14-7.91 (m, 1H), 7.77 (br d, J = 8.3 Hz, 2H), 7.34 (d, J = 8.5 Hz, 2H), 7.20 (q, J = 7.5 Hz, 2H), 7.07 (br s, 1H), 6.27 (br d, J = 15.6 Hz, 1H), 5.78-5.67 (m, 1H), 5.48-5.10 (m, 4H), 4.36 (s, 2H), 4.29-3.89 (m, 2H), 3.37 (s, 3H), 2.87 (s, 3H), 2.44-2.21 (m, 1H), 2.15 (s, 3H), 2.11- 1.98 (m, 1H) |
| 145 | 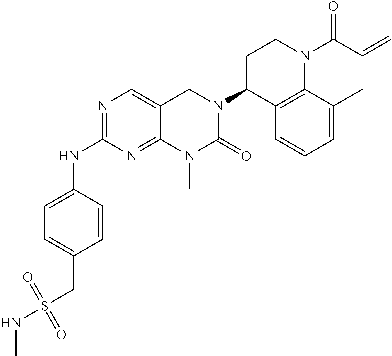 [SMILES: C=CC(=O)N1CC[C@H](N2Cc3cnc(Nc4ccc(CS(=O)(=O)NC)cc4)nc3N(C)C2=O)c2cccc(C)c21] | 562.4 | δ = 9.30 (s, 1H), 8.09-7.95 (m, 1H), 7.74 (br d, J = 8.6 Hz, 2H), 7.28 (d, J = 8.6 Hz, 2H), 7.21-7.15 (m, 2H), 7.11-7.00 (m, 1H), 6.67-6.58 (m, 1H), 6.26 (br d, J = 16.7 Hz, 1H), 5.76-5.67 (m, 1H), 4.19 (br d, J = 1.9 Hz, 4H), 4.04-3.82 (m, 1H), 3.40-3.32 (m, 4H), 2.56 (d, J = 4.8 Hz, 3H), 2.18-1.95 (m, 5H) |
| 146 | 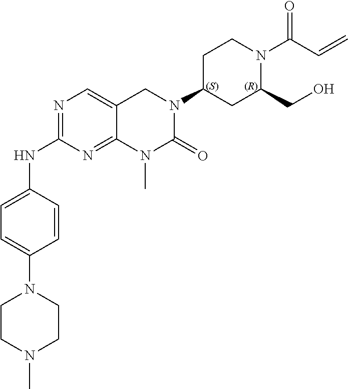 | 521.5 | δ = 9.64 (br s, 1H), 9.39 (br s, 1H), 8.07 (s, 1H), 7.60 (d, J = 8.9 Hz, 2H), 6.95 (d, J = 9.1 Hz, 2H), 6.81 (dd, J = 10.4, 16.6 Hz, 1H), 6.14 (dd, J = 2.4, 16.6 Hz, 1H), 5.71-5.63 (m, 1H), 4.35-4.19 (m, 4H), 4.15-4.04 (m, 3H), 3.73 (br d, J = 13.1 Hz, 2H), 3.57-3.46 (m, 4H), 3.25 (s, 3H), 3.21-3.10 (m, 2H), 2.96-2.81 (m, 5H), 2.08-1.82 (m, 2H), 1.80-1.66 (m, 2H) |
| 147 | 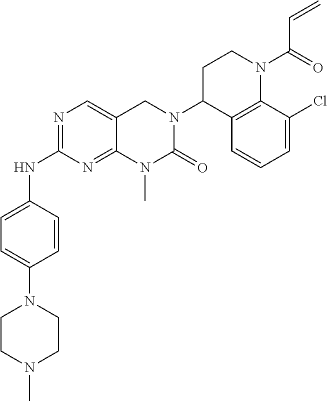 [SMILES: C=CC(=O)N1CCC(N2Cc3cnc(Nc4ccc(N5CCN(C)CC5)cc4)nc3N(C)C2=O)c2cccc(Cl)c21] | 573.5 | δ = 9.78-9.62 (m, 1H), 8.49 (s, 1H), 7.27-7.03 (m, 5H), 6.89 (d, J = 7.5 Hz, 1H), 6.50 (t, J = 7.7 Hz, 1H), 5.86 (br s, 1H), 5.55 (dd, J = 5.8, 9.1 Hz, 1H), 4.59 (q, J = 6.6 Hz, 2H), 4.38- 4.29 (m, 1H), 4.05 (d, J = 15.3 Hz, 1H), 3.98-3.90 (m, 1H), 3.78-3.67 (m, 1H), 3.65-3.52 (m, 3H), 3.20-3.12 (m, 4H), 3.03 (br d, J = 12.4 Hz, 1H), 2.95 (s, 3H), 2.91-2.86 (m, 2H), 2.14-2.01 (m, 1H), 1.99- 1.90 (m, 1H) |
| 148 | 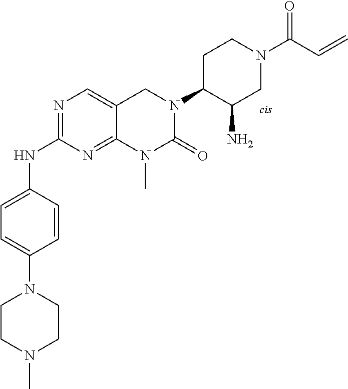 [SMILES: C=CC(=O)N1CC[C@H](N2Cc3cnc(Nc4ccc(N5CCN(C)CC5)cc4)nc3N(C)C2=O)[C@H](N)C1] | 506.5 | δ = 9.07 (s, 1H), 8.41-7.83 (m, 3H), 7.62 (d, J = 8.9 Hz, 2H), 6.95 (d, J = 9.1 Hz, 2H), 6.76 (br dd, J = 10.4, 16.7 Hz, 1H), 6.14 (dd, J = 2.0, 16.8 Hz, 1H), 5.83-5.63 (m, 1H), 4.77- 4.64 (m, 2H), 4.43-4.20 (m, 4H), 3.78-3.18 (m, 11H), 3.17- 2.91 (m, 2H), 2.88 (s, 3H), 2.04-1.77 (m, 2H) |
| 149 | 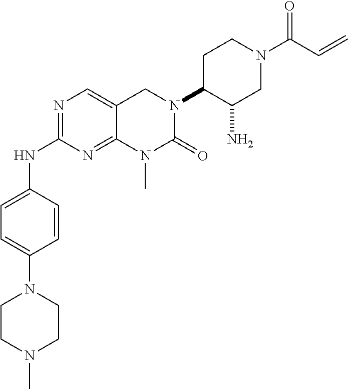 | 506.5 | δ = 9.04 (s, 1H), 8.10-7.88 (m, 3H), 7.63 (d, J = 8.9 Hz, 2H), 6.96 (d, J = 9.1 Hz, 2H), 6.80 (dd, J = 10.7, 16.7 Hz, 1H), 6.16 (dd, J = 2.3, 16.8 Hz, 1H), 5.75 (dd, J = 2.2, 10.5 Hz, 1H), 4.45-4.24 (m, 4H), 3.82 (br s, 1H), 3.30 (br s, 11H), 3.16-2.99 (m, 4H), 2.89 (s, 3H), 2.42-2.30 (m, 1H), 1.97- 1.85 (m, 1H) |
| 150 | 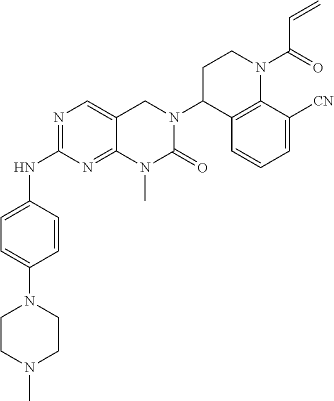 [SMILES: C=CC(=O)N1CCC(N2Cc3cnc(Nc4ccc(N5CCN(C)CC5)cc4)nc3N(C)C2=O)c2cccc(C#N)c21] | 564.5 | δ = 8.22 (s, 1H), 7.40-7.30 (m, 1H), 7.16-6.98 (m, 3H), 6.97-6.87 (m, 2H), 6.60-6.44 (m, 3H), 6.26-6.14 (m, 1H), 5.81-5.65 (m, 1H), 5.56 (br dd, J = 5.6, 10.9 Hz, 1H), 4.34- 4.26 (m, 1H), 4.24-4.07 (m, 1H), 4.01 (d, J = 15.1 Hz, 1H), 3.97-3.82 (m, 1H), 3.22-3.13 (m, 7H), 2.47-2.42 (m, 4H), 2.22 (s, 3H), 2.11-2.03 (m, 1H), 1.96-1.88 (m, 1H) |
| 151 | 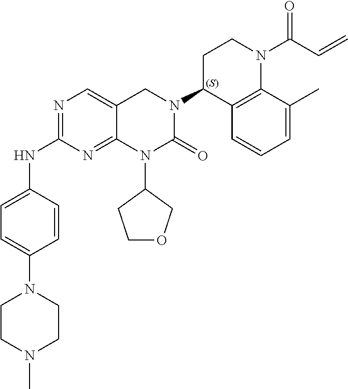 | 609.5 | δ = 10.06-9.18 (m, 1H), 9.06 (br s, 1H), 8.07-7.91 (m, 1H), 7.58 (br d, J = 8.9 Hz, 2H), 7.27- 7.12 (m, 2H), 7.08-6.88 (m, 3H), 6.27 (br d, J = 15.4 Hz, 1H), 5.87-5.45 (m, 2H), 5.42- 5.31 (m, 1H), 4.29-3.93 (m, 7H), 3.37 (br s, 8H), 2.89 (s, 3H), 2.46-2.37 (m, 1H), 2.34 (br s, 1H), 2.19-2.11 (m, 4H), 2.11-1.87 (m, 2H) |
| 152 | 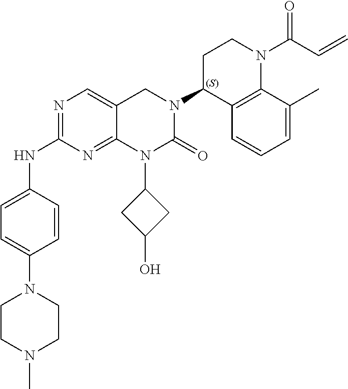 | 609.5 | 298K: δ = 9.17 (br s, 1H), 8.12- 7.82 (m, 1H), 7.61-7.44 (m, 2H), 7.33-7.08 (m, 2H), 6.88 (br d, J = 6.9 Hz, 4H), 6.38- 6.19 (m, 1H), 6.07-5.61 (m, 2H), 5.36-5.12 (m, 1H), 5.03- 4.87 (m, 1H), 4.79-4.57 (m, 1H), 4.31 (br dd, J = 1.7, 4.8 Hz, 1H), 4.18 (br d, J = 13.5 Hz, 1H), 4.09-3.80 (m, 3H), 3.06 (br s, 5H), 2.90-2.77 (m, 2H), 2.45 (br s, 4H), 2.22 (s, 4H), 2.14 (s, 3H) 273 + 80K: δ = 8.93-8.69 (m, 1H), 8.06-7.84 (m, 1H), 7.52 (br d, J = 8.8 Hz, 2H), 7.27- 7.10 (m, 2H), 7.02-6.91 (m, 1H), 6.87 (d, J = 8.9 Hz, 2H), 6.26 (br d, J = 16.1 Hz, 1H), 5.77-5.64 (m, 1H), 4.74-4.63 (m, 1H), 4.20-4.08 (m, 1H), 3.96-3.83 (m, 1H), 3.10 (br s, 7H), 2.89-2.76 (m, 2H), 2.49- 2.46 (m, 4H), 2.35-2.26 (m, 3H), 2.24 (s, 3H), 2.14 (s, 3H), 2.07-1.92 (m, 1H) |
| 153 | 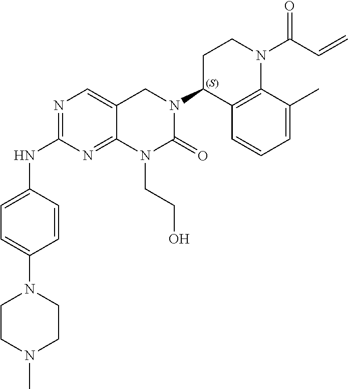 | 583.5 | δ = 9.30-9.18 (m, 1H), 8.38 (br d, J = 4.0 Hz, 1H), 8.04 (s, 1H), 7.89 (br d, J = 6.4 Hz, 1H), 7.62- 7.50 (m, 2H), 7.28-7.17 (m, 2H), 7.15 (br dd, J = 4.5, 5.4 Hz, 1H), 7.05-6.92 (m, 1H), 6.87 (br dd, J = 5.7, 8.4 Hz, 2H), 6.28 (br d, J = 16.6 Hz, 1H), 5.79-5.64 (m, 1H), 4.15- 3.99 (m, 3H), 3.67-3.58 (m, 3H), 3.05 (br s, 5H), 2.45 (br s, 4H), 2.22 (s, 3H), 2.17-2.10 (m, 3H), 2.04 (br s, 1H) |
| 154 | 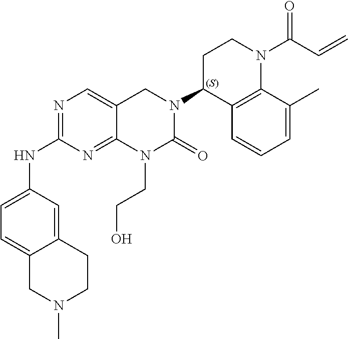 | 554.5 | 298K: δ = 9.46-9.30 (m, 1H), 8.11-7.90 (m, 1H), 7.65-7.50 (m, 1H), 7.43-7.31 (m, 1H), 7.29-6.88 (m, 5H), 6.28 (br d, J = 15.9 Hz, 1H), 6.06-5.63 (m, 2H), 4.84-4.58 (m, 2H), 4.38-4.25 (m, 1H), 4.19-3.98 (m, 4H), 3.74-3.55 (m, 4H), 2.79 (br d, J = 3.9 Hz, 2H), 2.56 (br s, 2H), 2.32 (s, 3H), 2.19- 1.87 (m, 5H) 273 + 80K: δ = 9.78 (br s, 1H), 9.37-9.24 (m, 1H), 8.10-7.91 (m, 1H), 7.76 (s, 1H), 7.58- 7.48 (m, 1H), 7.25-6.99 (m, 5H), 6.30-6.21 (m, 1H), 5.79- 5.61 (m, 1H), 4.48-3.88 (m, 8H), 3.68 (br t, J = 6.5 Hz, 3H), 3.15-3.06 (m, 3H), 2.95 (s, 3H), 2.18-1.96 (m, 5H) |
| 155 | 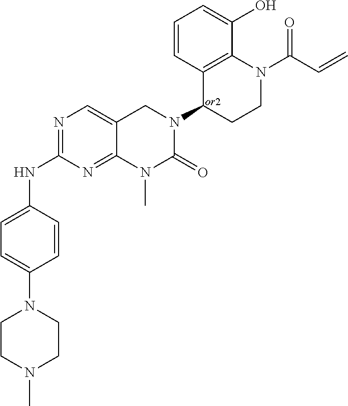 [SMILES: C=CC(=O)N1CC[C@@H](N2Cc3cnc(Nc4ccc(N5CCN(C)CC5)cc4)nc3N(C)C2=O)c2cccc(O)c21] | 555.5 | 298K: δ = 9.91 (br s, 1H), 9.25 (s, 1H), 7.98 (br s, 1H), 7.56 (br d, J = 8.9 Hz, 2H), 7.06 (t, J = 7.8 Hz, 1H), 6.92-6.81 (m, 3H), 6.64 (br s, 1H), 6.40 (br s, 1H), 6.22-6.14 (m, 1H), 5.61 (br d, J = 8.8 Hz, 2H), 4.18 (br s, 3H), 3.32-3.31 (m, 3H), 3.12- 3.00 (m, 5H), 2.47-2.42 (m, 4H), 2.22 (s, 4H), 1.97 (br s, 1H) 273 + 80K: δ = 9.58-9.42 (m, 1H), 8.91 (s, 1H), 7.93 (s, 1H), 7.54 (d, J = 9.1 Hz, 2H), 7.12- 7.00 (m, 1H), 6.91-6.80 (m, 3H), 6.66 (d, J = 7.5 Hz, 1H), 6.42 (br dd, J = 10.3, 16.9 Hz, 1H), 6.16 (dd, J = 2.3, 16.8 Hz, 1H), 5.63-5.47 (m, 2H), 4.18 (br d, J = 13.6 Hz, 1H), 4.02- 3.76 (m, 2H), 3.32 (s, 3H), 3.14 (s, 1H), 3.00 (br s, 4H), 2.48- 2.45 (m, 4H), 2.24 (s, 4H), 2.08- 1.96 (m, 1H) |
| 156 | 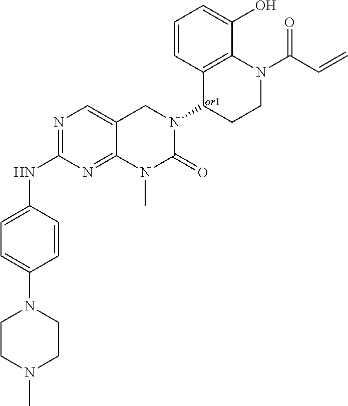 | 555.5 | δ = 10.02-9.82 (m, 1H), 9.23 (s, 1H), 8.20 (s, 1H), 8.02-7.89 (m, 1H), 7.55 (br d, J = 8.5 Hz, 2H), 7.15-6.99 (m, 1H), 6.90- 6.48 (m, 5H), 6.23-6.08 (m, 1H), 5.67-5.53 (m, 1H), 4.75- 3.64 (m, 4H), 3.30 (br s, 3H), 3.06-3.03 (m, 4H), 2.46-2.43 (m, 4H), 2.26-1.87 (m, 5H) |
| 157 | 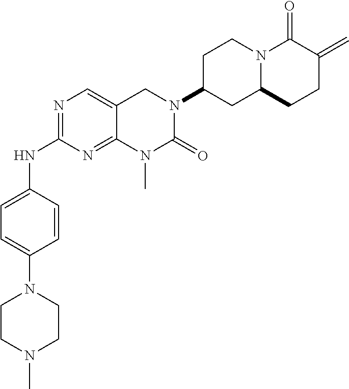 | 517.4 | δ = 9.71-9.51 (m, 1H), 9.47- 9.22 (m, 1H), 8.02 (s, 1H), 7.61 (d, J = 9.0 Hz, 2H), 7.01-6.89 (m, 2H), 5.94-5.87 (m, 1H), 5.29-5.25 (m, 1H), 4.76-4.63 (m, 1H), 4.52-4.37 (m, 1H), 4.25 (s, 2H), 3.74 (br d, J = 13.7 Hz, 2H), 3.27 (s, 3H), 3.22- 3.12 (m, 2H), 2.97-2.81 (m, 5H), 2.74-2.53 (m, 2H), 2.50- 2.31 (m, 4H), 2.10-1.98 (m, 1H), 1.79-1.45 (m, 5H) |
| 158 | 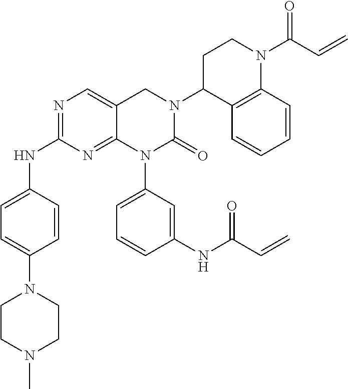 [SMILES: C=CC(=O)Nc1cccc(N2C(=O)N(C3CCN(C(=O)C=C)c4ccccc43)Cc3cnc(Nc4ccc(N5CCN(C)CC5)cc4)nc32)c1] | 670.4 | ¹H NMR (400 MHz, DMSO-d6) δ = 10.34 (s, 1H), 9.21-9.15 (m, 1H), 8.31-8.28 (m, 1H), 8.08 (s, 1H), 7.85 (br d, J = 8.6 Hz, 1H), 7.65 (s, 1H), 7.50- 7.43 (m, 1H), 7.35-7.21 (m, 4H), 7.19-7.11 (m, 2H), 7.10- 7.03 (m, 1H), 6.73-6.61 (m, 1H), 6.58-6.51 (m, 2H), 6.44 (dd, J = 10.1, 17.1 Hz, 1H), 6.31-6.21 (m, 2H), 5.81-5.72 (m, 2H), 5.72-5.64 (m, 1H), 4.45-4.38 (m, 1H), 4.24-4.15 (m, 1H), 4.11-3.99 (m, 1H), 3.79-3.68 (m, 1H), 2.98-2.90 (m, 4H), 2.45-2.37 (m, 4H), 2.29-2.10 (m, 5H) |
| 159 | 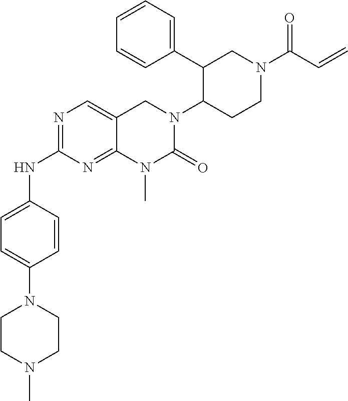 [SMILES: C=CC(=O)N1CCC(N2Cc3cnc(Nc4ccc(N5CCN(C)CC5)cc4)nc3N(C)C2=O)C(c2ccccc2)C1] | 567.2 | δ: 9.15 (br s, 1 H) 8.21 (s, 1 H) 7.52 (br d, J = 8.38 Hz, 2 H) 7.42 (s, 1 H) 7.06-7.29 (m, 5 H) 6.85 (br d, J = 8.88 Hz, 2 H) 6.16 (br dd, J = 16.32, 7.82 Hz, 1 H) 5.70-5.85 (m, 1 H) 5.63 (br d, J = 10.13 Hz, 1 H) 4.68-4.82 (m, 2 H) 4.22-4.44 (m, 2 H) 3.86 (br d, J = 13.76 Hz, 2 H) 3.28 (br s, 3 H) 3.04 (br s, 4 H) 2.66-2.75 (m, 2 H) 2.45 (br d, J = 4.38 Hz, 4 H) 2.22 (s, 3 H) 1.94-2.09 (m, 1 H) 1.75 (br d, J = 11.63 Hz, 1 H) |
| 160 | 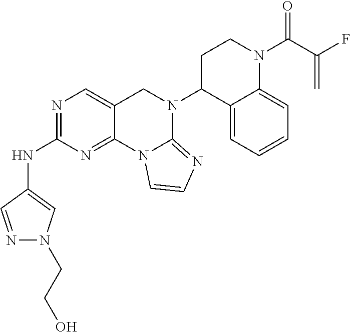 [SMILES: C=C(F)C(=O)N1CCC(N2Cc3cnc(Nc4cnn(CCO)c4)nc3-n3ccnc32)c2ccccc21] | 502.1 | δ = 10.02-9.67 (m, 1H), 8.42- 8.35 (m, 1H), 8.29-8.03 (m, 2H), 7.84-7.59 (m, 3H), 7.50- 7.41 (m, 1H), 7.36-7.25 (m, 2H), 7.23-7.16 (m, 1H), 5.42- 5.15 (m, 2H), 4.98-4.83 (m, 1H), 4.76-4.64 (m, 1H), 4.61- 4.45 (m, 2H), 4.24-4.07 (m, 2H), 3.90-3.66 (m, 4H), 2.31- 2.21 (m, 2H) |

### Example 2: HTRF-Based EGFR Biochemical Assays

To measure the IC₅₀ values of compounds herein against EGFR, a Z′-LYTE assay (ThermoFisher) was used. Briefly, 2.5 μL of different concentrations of the compounds in 4% DMSO were added to 5 μL kinase/peptide mixture in each well of a 384-well plate (Corning Cat. #3676). 2.5 μL of 4× ATP solution (4× ATP, 50 mM HEPES, pH 7.5, 0.01% BRIJ-35, 10 mM MgCl₂, 1 mM EGTA) were added to each well. The plate was shaken for 30 seconds, and then incubated at room temperature for 60 minutes. 5 μL of a 1:100000 dilution of Development Reagent A was added to each well. The plate was then incubated for 60 mins at room temperature. 5 μL of the stop reagent was then added. The plate was subsequently read on a fluorescence plate reader, and the emissions ratio was calculated to determine the ratio of Ser/Thr 07 phosphorylated by the reaction. Emissions Ratio═Coumarin Emission (443 nm)/Flourescein Emission (520 nm).

HTRF-based EGFR assay data are provided below in Table 4.

TABLE 4

| Compound No. | L858R HTRF (nM) | L858R/T790M HTRF (nM) |
|---|---|---|
| 154 | n.d. | n.d. |
| 153 | 0.88 | >1000.00 |
| 151 | 0.86 | >1000.00 |
| 152 | 1.17 | >1000.00 |
| 144 | 1.57 | >1000.00 |
| 145 | 1.77 | >1000.00 |
| 146 | >1000.00 | >1000.00 |
| 147 | >1000.00 | >1000.00 |
| 141 | >1000.00 | >1000.00 |
| 140 | 0.97 | >1000.00 |
| 143 | >1000.00 | >1000.00 |
| 150 | >1000.00 | >1000.00 |
| 137 | >1000.00 | >1000.00 |
| 136 | >1000.00 | >1000.00 |
| 138 | >1000.00 | >1000.00 |
| 142 | >1000.00 | >1000.00 |
| 148 | >1000.00 | >1000.00 |
| 149 | >1000.00 | >1000.00 |
| 135 | 0.89 | >1000.00 |
| 139 | >1000.00 | >1000.00 |
| 134 | 38.94 | >1000.00 |
| 131 | 0.94 | >1000.00 |
| 155 | 12.25 | 85.72 |
| 156 | 0.18 | 1.18 |
| 133 | 1.13 | 10.0 |
| 130 | 42.99 | >1000.00 |
| 132 | >1000.00 | >1000.00 |
| 129 | n.d. | n.d. |
| 127 | 1.20 | >700.00 |
| 128 | 0.9 | >700.00 |
| 121 | >100.00 | >100.00 |
| 125 | >100.00 | >100.00 |
| 120 | >100.00 | >100.00 |
| 119 | >100.00 | >100.00 |
| 118 | >100.00 | >100.00 |
| 117 | >100.00 | >100.00 |
| 122 | >100.00 | >100.00 |
| 159 | >100.00 | >100.00 |
| 157 | >1000.00 | >1000.00 |
| 124 | >100.00 | >100.00 |
| 123 | 62.92 | >100.00 |
| 001A | >100.00 | >100.00 |
| 126 | >100.00 | >100.00 |
| 158 | 1.07 | 0.71 |

n.d. = not determined

### Example 3: Ba/F3 cell proliferation models

The EGFR mutant L858R Ba/F3 cells have been previously described (Zhou, W., et al. Nature 462, 2009, 1070-1074). The EGFR C797S and C₇₇₅S mutations were introduced via site directed mutagenesis using the Quick Change Site-Directed Mutagenesis kit into a vector containing EGFR L858R mutation (Stratagene; La Jolla, CA) according to the manufacturer's instructions. All constructs were confirmed by DNA sequencing. The constructs were then shuttled into the retroviral vector JP1540 by either using the Cre-recombination system (Agilent Technologies, Santa Clara, CA) or the In-fusion HD Cloning kit (Takara Bio USA, Inc.; Mountain view, CA). Ba/F3 cells were then infected with retrovirus per standard protocols, as described previously (Zhou, et al, Nature 2009). Stable clones were obtained by selection in puromycin (2 μg/ml). All BaF/3 mutant cells were maintained in RPMI 1640 (Cellgro; Mediatech Inc., Herndon, CA) supplemented with 10% FBS, 100 units/mL penicillin, 100 units/mL streptomycin.

Growth and inhibition of growth was assessed by the Cell Titer Glo assay (Promega, Madison, WI) and was performed according to the manufacturer's instructions. The Cell Titer Glo assay is a luminescence-based method used to determine the number of viable cells based on quantitation of the ATP present, which is directly proportional to the amount of metabolically active cells present. Ba/F3 cells of different EGFR genotypes were exposed to compounds for 72 hours and the number of cells used per experiment was determined empirically as has been previously established (Zhou, et al., Nature 2009). All experimental points were set up in triplicates in 384-well plates. The luminescent signal was detected using a spectrometer and the data was graphically displayed using GraphPad Prism version 5.0 for Windows, (GraphPad Software; www.graphpad.com). The curves were fitted using a non-linear regression model with a sigmoidal dose response.

Ba/F3 cells assay data are provided below in Table 5 and Table 6. For inhibitory activity against EGFR mutants, the following designations are used: <0.1 μM=A, 0.1 μM˜0.5 μM=B, 0.5 μM˜2.5 μM=C, ≥2.5 μM=D

TABLE 5

| Compound No. | L858R BaF3 (μM) | L858R/C797S (μM) |
|---|---|---|
| 001 | B | B |
| 036 | C | D |
| 037 | D | D |
| 019 | B | C |

TABLE 6

| Compound No. | L858R BaF3 (μM) | L858R/C797S (μM) |
|---|---|---|
| 046 | D | D |
| 047 | B | B |
| 048 | A | A |
| 049 | A | A |
| 050 | D | D |
| 158 | A | A |
| 051 | B | C |
| 052 | A | B |
| 053 | D | D |
| 054 | D | D |
| 055 | A | A |
| 056 | A | A |
| 057 | A | A |
| 058 | A | A |
| 059 | D | D |
| 060 | D | D |
| 061 | A | A |
| 062 | A | A |
| 063 | A | A |
| 064 | A | A |
| 065 | C | C |
| 066 | A | A |
| 067 | A | A |
| 068 | A | A |
| 069 | A | A |
| 070 | A | A |
| 071 | A | A |
| 072 | A | A |
| 073 | A | A |
| 074 | A | A |
| 075 | A | A |
| 076 | A | A |
| 077 | A | A |
| 078 | A | A |
| 079 | A | A |
| 080 | A | A |
| 081 | A | A |
| 082 | A | B |
| 082 | A | A |
| 084 | D | D |
| 085 | A | A |
| 086 | A | A |
| 087 | A | A |
| 088 | A | A |
| 089 | D | D |
| 090 | C | C |
| 091 | B | B |
| 092 | D | D |
| 093 | A | A |
| 094 | D | D |
| 095 | A | A |
| 096 | D | D |
| 097 | B | C |
| 098 | A | A |
| 099 | A | A |
| 100 | A | A |
| 101 | D | D |
| 102 | A | A |
| 103 | D | D |
| 104 | D | D |
| 105 | D | D |
| 106 | D | D |
| 107 | D | D |
| 108 | C | D |
| 109 | A | A |
| 110 | A | A |
| 111 | B | C |
| 112 | D | D |
| 113 | A | B |
| 001A | D | D |
| 159 | C | D |
| 117 | D | D |
| 118 | B | C |
| 119 | D | D |
| 120 | D | D |
| 121 | D | D |
| 122 | B | C |
| 123 | D | D |
| 124 | D | D |
| 125 | D | D |
| 126 | D | D |
| 127 | A | A |
| 128 | A | A |
| 129 | C | D |
| 130 | A | B |
| 131 | A | A |
| 132 | B | B |
| 133 | A | A |
| 134 | A | B |
| 157 | D | D |
| 135 | A | A |
| 136 | D | D |
| 137 | D | D |
| 138 | D | D |
| 139 | B | C |
| 140 | A | A |
| 141 | B | B |
| 142 | C | D |
| 143 | C | D |
| 144 | A | A |
| 145 | A | A |
| 146 | C | D |
| 147 | C | D |
| 148 | D | D |
| 149 | D | D |
| 150 | C | C |
| 151 | A | A |
| 152 | A | A |
| 153 | A | A |
| 154 | A | A |
| 155 | A | B |
| 156 | A | A |

The disclosed subject matter is not to be limited in scope by the specific embodiments and examples described herein. Indeed, various modifications of the disclosure in addition to those described will become apparent to those skilled in the art from the foregoing description and accompanying FIGURES. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula I-H: 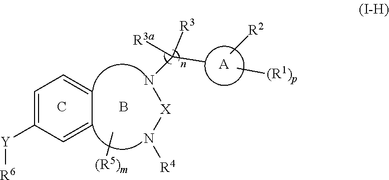
or a pharmaceutically acceptable salt thereof;
wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, D, halo, CN, OR⁷, NO₂, C₁-C₆ alkyl, C₁-C₆ haloalkyl, C₁-C₆ alkyl-N(R⁷)₂, C₁-C₆ alkyl-OH, C(O)OH, N(R⁷)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
p is 1 or 2;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein alkyl, cycloalkyl, heterocycloalkyl, aryl, and heteroaryl are each optionally substituted with R¹;
each R⁵ is independently selected from the group consisting of C₁-C₆ alkyl, ═O, halo, OR⁷, and N(R⁷)₂;
alternatively, R⁴ and R⁶ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 8-10 membered bicyclic ring, and 3-10 membered heterocycloalkenyl all of which are optionally substituted 1, 2, 3, 4, or 5 times with R^{6a};
each R^{6aa} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkyl, 3-10 membered heterocycloalkenyl, and 5-10 membered heteroaryl,
wherein C₁-C₆ alkyl, OC₁-C₆ alkyl, N(C₁-C₆ alkyl)₂, heterocycloalkyl, heterocycloalkenyl, and heteroaryl are optionally substituted with halo, OH, C₁-C₆ alkyl, N(C₁-C₆ alkyl)₂, SO₂N(R⁷)₂, SO₂(C₁-C₆ alkyl), and 3-10 membered heterocycloalkyl, wherein 3-10 membered heterocycloalkyl is optionally substituted 1 or 2 times with R^{6aa};
R^{6aa} is selected from C₁-C₆ alkyl, C₁-C₆ haloalkyl, C₁-C₆ alkoxy, and C₁-C₆ alkyl-OH;
each R⁷ is independently selected from the group consisting of H, OH, halo, C₁-C₆ alkyl, C₁-C₆ alkyl-OH, C₆-C₁₀ aryl, 5-10 membered heteroaryl, C₃-C₁₀ cycloalkyl, and 3-10 membered heterocycloalkyl;
R⁸ is selected from OH, C(O)C₁-C₆ alkyl-OH, SO₂C₁-C₆ alkyl, SO₂C₃-C₆ cycloalkyl, halo, C(O)C₁-C₆ alkyl, C₁-C₆ alkyl, NHC(O)C₁-C₆ alkyl, and C₁-C₆ alkoxy;
R² is selected from the group consisting of: 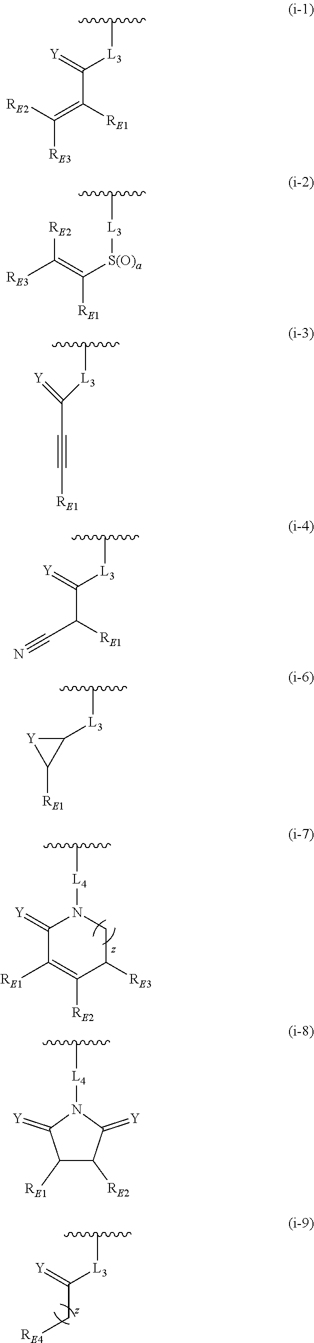 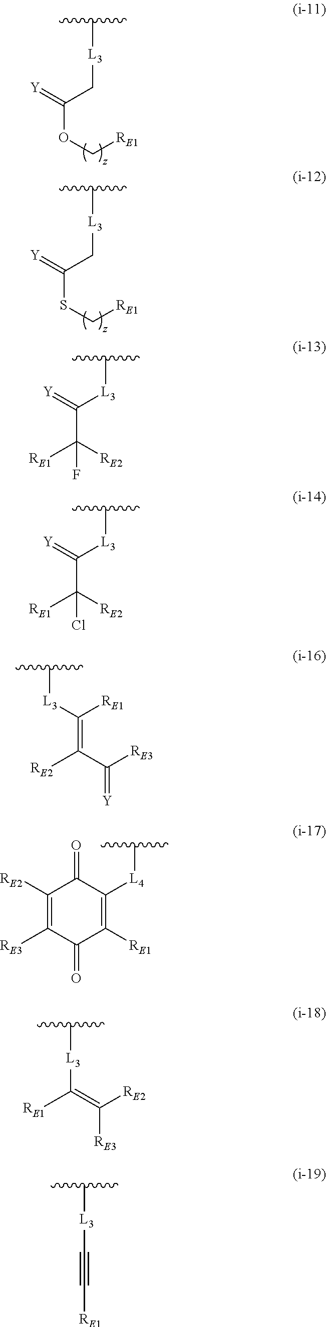 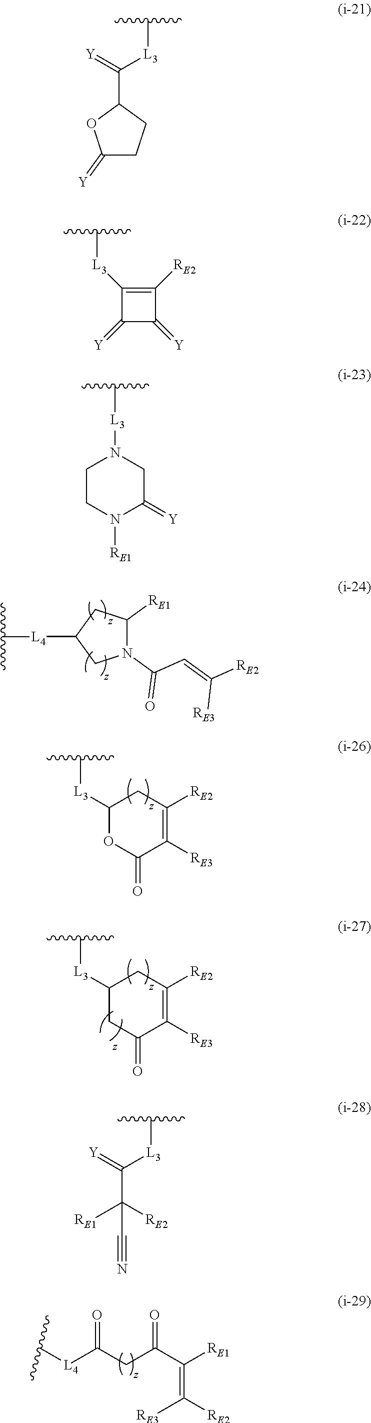 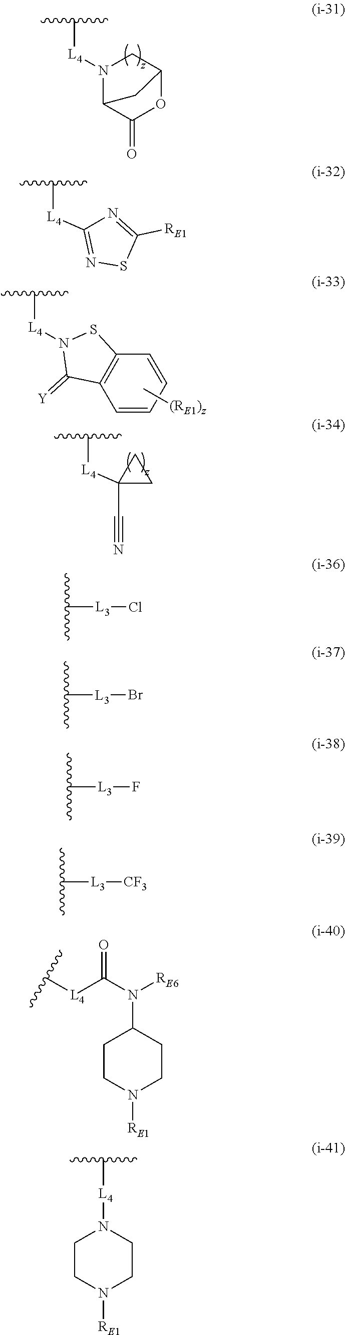 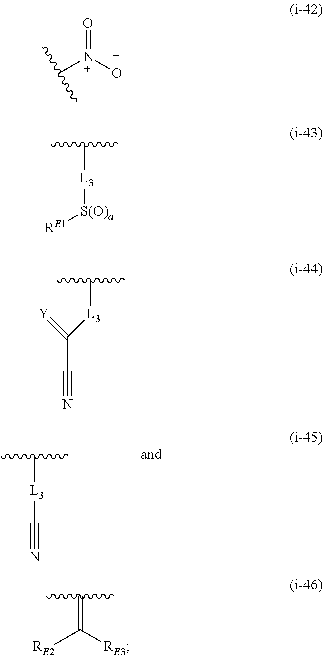
L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a} C(S)—, —C(S)NR_{L3a} —, trans-CR_{L3b}CR_{L3b}—, cis-CR_{L3b}CR_{L3b}—, —C═C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a} —, or —NR_{L3a} S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C(O)OH, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{E}E groups, together with the atom to which they are attached, form 4-7 membered heterocycloalkyl;
R_{E6} is hydrogen, C₁-C₆ alkyl, or a nitrogen protecting group;
each Y is independently 0, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 0, 1, 2, or 3.

2. The compound of claim 1, wherein the Formula I-H is a compound of Formula I-G: 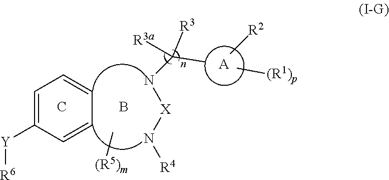
or a pharmaceutically acceptable salt thereof;
wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, D, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
p is 1 or 2;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R⁸;
each R⁵ is independently selected from the group consisting of C₁-C₆ alkyl, halo, OR⁷, and N(R⁷)₂;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, 8-10 membered bicyclic ring, and 3-10 membered heterocycloalkenyl all of which are optionally substituted 1, 2, 3, 4, or 5 times with R^{6a};
each R^{6a} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkyl, 3-10 membered heterocycloalkenyl, and 5-10 membered heteroaryl,
wherein C₁-C₆ alkyl, OC₁-C₆ alkyl, N(C₁-C₆ alkyl)₂, heterocycloalkyl, heterocycloalkenyl, and heteroaryl are optionally substituted with halo, OH, C₁-C₆ alkyl, N(C₁-C₆ alkyl)₂, SO₂N(R⁷)₂, and 3-10 membered heterocycloalkyl, wherein 3-10 membered heterocycloalkyl is optionally substituted 1 or 2 times with R^{6aa};
R^{6aa} is C₁-C₆ alkyl;
each R⁷ is independently selected from the group consisting of H, OH, halo, C₁-C₆ alkyl, C₁-C₆ alkyl-OH, C₆-C₁₀ aryl, 5-10 membered heteroaryl, C₃-C₁₀ cycloalkyl, and 3-10 membered heterocycloalkyl;
R⁸ is selected from C(O)C₁-C₆ alkyl-OH, SO₂C₁-C₆ alkyl, SO₂C₃-C₆ cycloalkyl, halo, C(O)C₁-C₆ alkyl, C₁-C₆ alkyl, NHC(O)C₁-C₆ alkyl, and C₁-C₆ alkoxy;
R² is selected from the group consisting of: 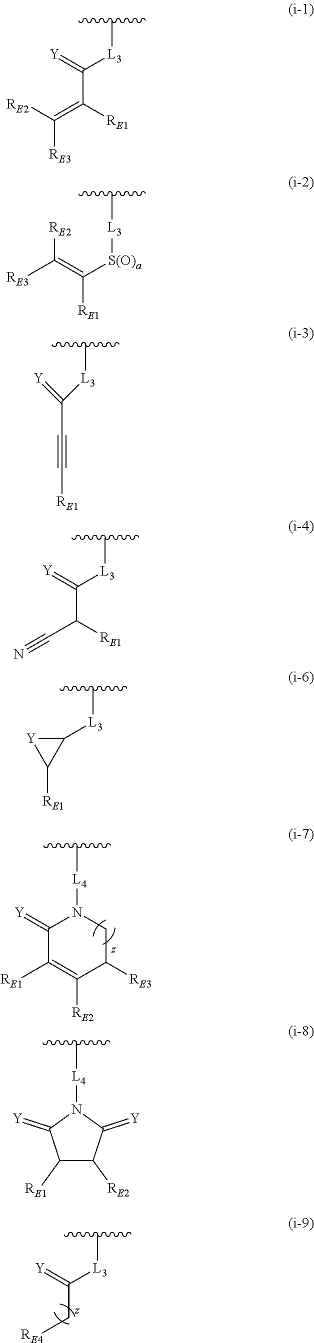 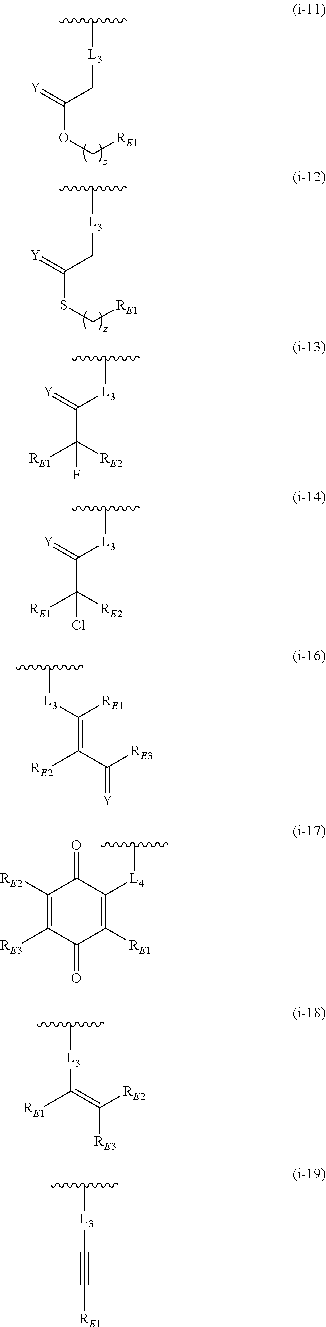 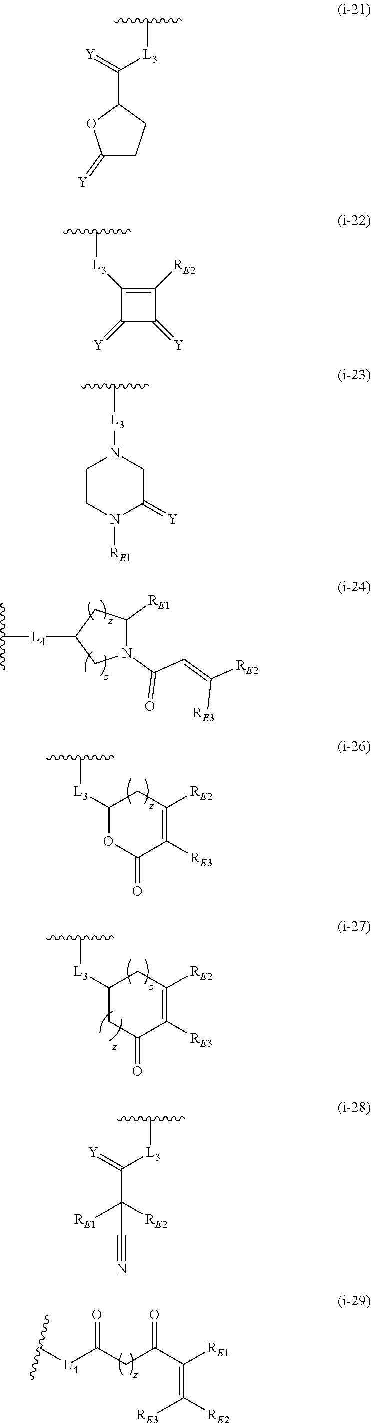 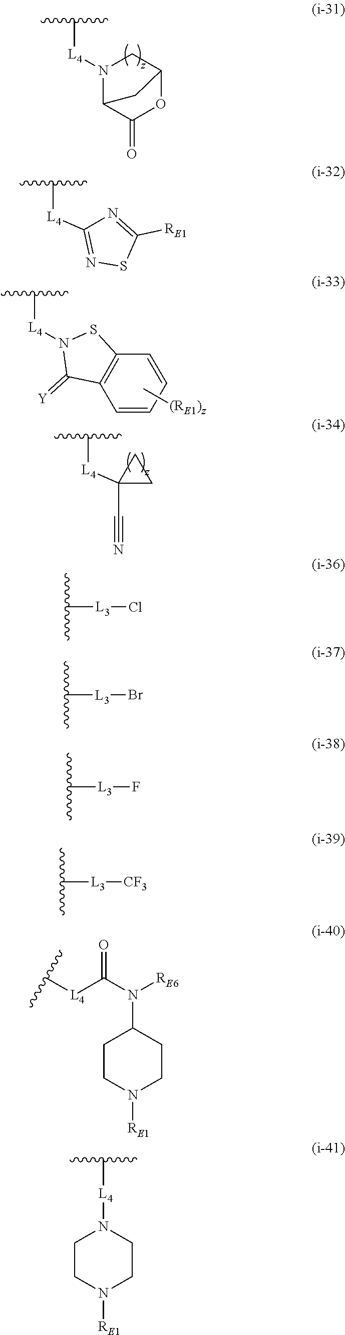 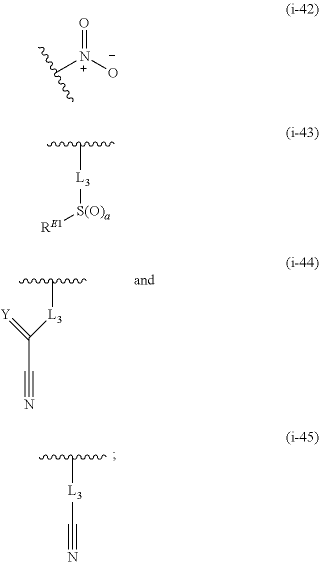
L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)—, —NR_{L3a} C(S)—, —C(S)NR_{L3a} —, trans-CR_{L3b}═CR_{L3b}—, cis-CR_{L3b}═CR_{L3b}—, —C≡C—, —S(O)—, 3S(O)O—, OS(O)—, —S(O)NR_{L3a}—, —NR_{L3a}S(O)—, —S(O)₂—, —S(O)₂O—, OS(O)₂—, —S(O)₂NR_{L3a} —, or —NR_{L}3aS(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

3. The compound of claim 2, wherein the Formula I-G is a compound of Formula I: 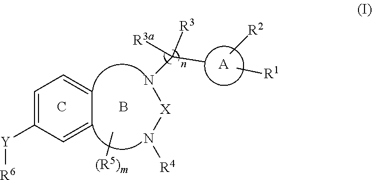
or a pharmaceutically acceptable salt thereof;
wherein:
X is C(O), C(S), C(NH), or SO₂;
Y is O, S, or NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, C₃-C₁₀ cycloalkenyl, 3-10 membered heterocycloalkenyl, and 6-10 membered bicyclic ring;
B is 5-7 membered ring;
C is pyrimidine, pyridine, or pyridazine;
R¹ is selected from the group consisting of H, halo, CN, OR⁷, NO₂, C₁-C₆ alkyl, C₁-C₆ haloalkyl, C₁-C₆ alkyl-N(R⁷)₂, C₁-C₆ alkyl-OH, N(R⁷)₂, NHC(O)R⁷, C(O)N(R⁷)₂, NHC(O)N(R⁷)₂, SO₂N(R⁷)₂, NHSO₂R⁷, OC(O)N(R⁷)₂, NHC(O)OR⁷, C₆-C₁₀ aryl, 5-10 membered heteroaryl, C₃-C₁₀ cycloalkyl, 3-10 membered heterocycloalkyl, C₃-C₁₀ cycloalkenyl, and 4-10 membered heterocycloalkenyl;
R³ and R^{3a} are each independently selected from the group consisting of H, halo, and C₁-C₆ alkyl;
alternatively, R³ and R^{3a} optionally combine to form C₃-C₁₀ cycloalkyl or 3-10 membered heterocycloalkyl;
n is 0, 1, or 2;
R⁴ is selected from the group consisting of H, C₁-C₆ alkyl, C₃-C₁₀ cycloalkyl, 3-10 membered heterocycloalkyl, C₆-C₁₀ aryl, and 5-10 membered heteroaryl; wherein cycloalkyl, heterocycloalkyl, aryl, and heteroaryl are each optionally substituted with R¹;
each R⁵ is independently selected from the group consisting of C₁-C₆ alkyl, halo, OR⁷, and N(R⁷)₂;
alternatively, R⁴ and R⁶ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl, 5-10 membered heteroaryl, C₃-C₁₀ cycloalkyl, 3-10 membered heterocycloalkyl, C₃-C₁₀ cycloalkenyl, and 3-10 membered heterocycloalkenyl all of which are substituted with 3-10 membered heterocycloalkyl, wherein the heterocycloalkyl is optionally substituted with C₁-C₆ alkyl;
each R⁷ is independently selected from the group consisting of H, OH, halo, C₁-C₆ alkyl, C₆-C₁₀ aryl, 5-10 membered heteroaryl, C₃-C₁₀ cycloalkyl, and 3-10 membered heterocycloalkyl;
R⁸ is selected from C(O)C₁-C₆ alkyl-OH, SO₂C₁-C₆ alkyl, SO₂C₃-C₆ cycloalkyl, halo, C(O)C₁-C₆ alkyl, C₁-C₆ alkyl, and C₁-C₆ alkoxy;
R² is selected from the group consisting of: 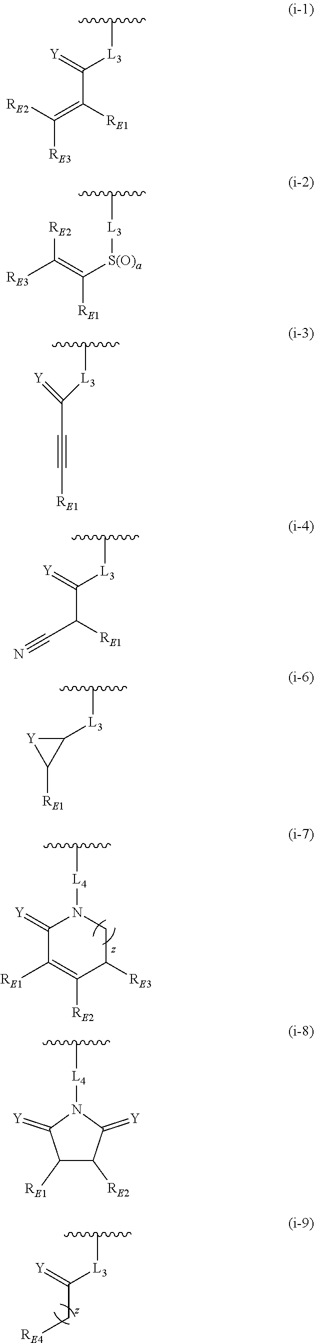 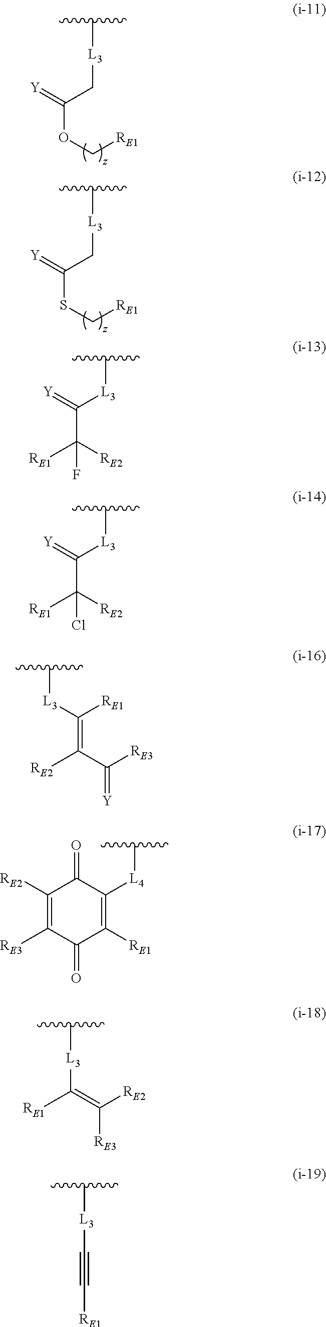 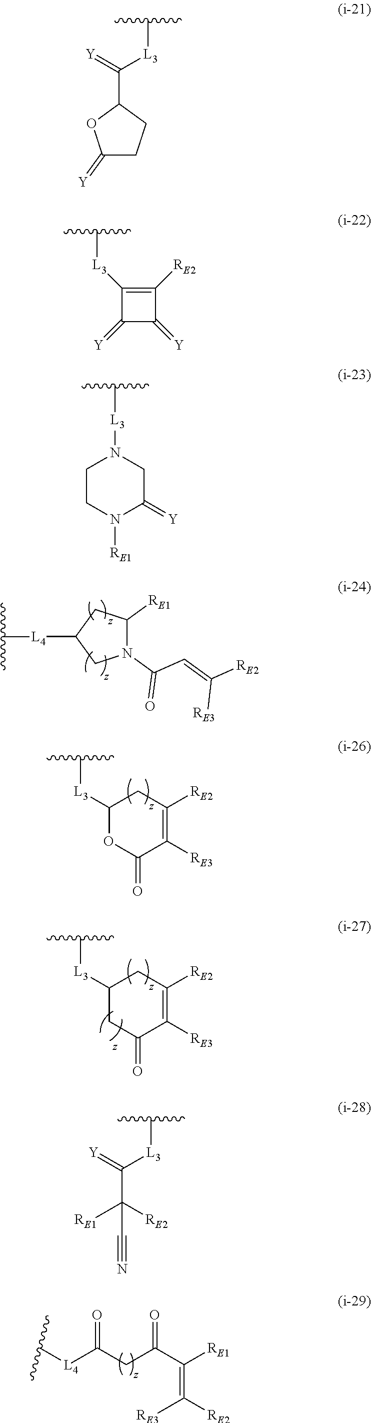 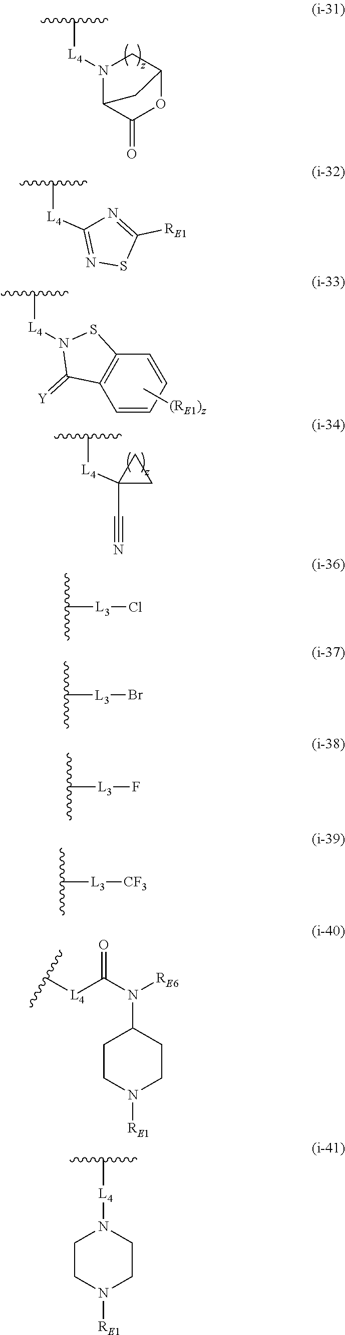 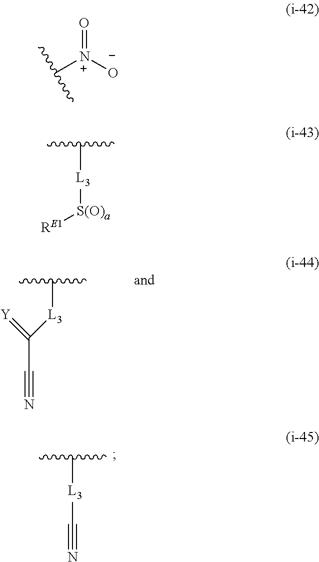
L₃ is a bond, —NH—, —N(C₁-C₄ alkyl)-, or C₁-C₄ alkylene, optionally wherein one or more carbons is independently replaced with —C(O)—, —O—, —S—, —NR_{L3a}—, —NR_{L3a}C(O)—, —C(O)NR_{L3a}—, —SC(O)—, —C(O)S—, —OC(O)—, —C(O)O—, —NR_{L3a}C(S)—, —C(S)NR_{L3a}—, trans-CR_{L3b}═CR_{L3b}—, cis-CR_{L3b}═CR_{L3b}—, cis-CR_{L3b}═CR_{L3b}—, —C—C—, —S(O)—, —S(O)O—, —OS(O)—, —S(O)NR_{L3a}—, —NR_{L3a}S(O)—, —S(O)₂—, —S(O)₂O—, —OS(O)₂—, —S(O)₂NR_{L3a}—, or —NR_{L3a}S(O)₂—;
R_{L3a} is hydrogen, C₁-C₆ alkyl optionally substituted with R⁹, or a nitrogen protecting group;
R_{L3b} is independently, at each occurrence, selected from the group consisting of hydrogen, halogen, C₁-C₆ alkyl, C₂-C₆ alkenyl, C₂-C₆ alkynyl, 3-8 membered cycloalkyl, 3-12 membered heterocycloalkyl, 6-10 membered aryl, and 5-8 membered heteroaryl, wherein alkyl, alkenyl, alkynyl, cycloalkyl, heterocyclyl, aryl, and heteroaryl are optionally substituted with one, two, or three R⁹;
or, alternatively, two R_{L3b} groups, together with the atoms to which they are attached, form a 3-8 membered cycloalkyl or 4-7 membered heterocycloalkyl, both of which are optionally substituted with one, two, or three R⁹;
L₄ is a bond or C₁-C₆ alkyl optionally substituted with one, two, or three R⁹;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{EE} groups, together with the atom to which they are attached, form 4-7 membered heterocycloalkyl;
R_{E6} is hydrogen, C₁-C₆ alkyl, or a nitrogen protecting group;
each Y is independently 0, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

4. The compound of claim 2, wherein
p is 2;
R⁶ is 8-10 membered bicyclic ring optionally substituted 1, 2, 3, 4, or 5 times with R^{6a};
each R^{6a} is independently selected from the group consisting of halo, CN, C₁-C₆ alkyl, C₁-C₆ haloalkyl, OC₁-C₆ alkyl, N(C₁-C₆ alkyl)₂, SO₂N(R⁷)₂, 3-10 membered heterocycloalkenyl, and 5-10 membered heteroaryl, wherein C₁-C₆ alkyl, OC₁-C₆ alkyl, N(C₁-C₆ alkyl)₂, heterocycloalkenyl, and heteroaryl are optionally substituted with halo, OH, N(C₁-C₆ alkyl)₂, SO₂N(R⁷)₂, 3-10 membered heterocycloalkyl, wherein 3-10 membered heterocycloalkyl is optionally substituted 1 or 2 times with R^{6aa};
R^{6aa} is C₁-C₆ alkyl;
each R⁷ is independently selected from H and C₁-C₆ alkyl-OH; and
R⁸ is NHC(O)C₁-C₆ alkyl.

5. The compound of any one of claims 1-4, wherein
X is C(O);
Y is NH;
A is selected from the group consisting of C₂-C₆ alkenyl, C₆-C₁₀ aryl, 5-10 membered heteroaryl, C₃-C₁₀ cycloalkyl, 3-10 membered heterocycloalkyl, and 6-10 membered bicyclic ring;
B is 5-7 membered heterocyclic ring;
C is pyrimidine, pyridine, or pyridazine;
R¹ is selected from the group consisting of H, halo, CN, C₁-C₆ alkyl, C₁-C₆ haloalkyl, O—C₁-C₆ alkyl, 0(3-10 membered heterocycloalkyl), NHSO₂C₁-C₆ alkyl, and NH(3-10 membered heterocycloalkyl);
R³ and R^{3a} are each independently selected from the group consisting of H and C₁-C₆ alkyl;
alternatively, R³ and R^{3a} optionally combine to form C₃-C₁₀ cycloalkyl;
n is 0, 1, or 2;
R⁴ is C₁-C₆ alkyl or 3-10 membered heterocycloalkyl optionally substituted with R⁸;
each R⁵ is independently selected from the group consisting of C₁-C₆ alkyl and halo;
alternatively, R⁴ and R⁵ optionally combine to form a bridged C₃-C₁₀ cycloalkyl or 3-10 membered heterocycloalkyl;
alternatively, two R⁵ optionally combine to form C₃-C₁₀ cycloalkyl or 3-10 membered heterocycloalkyl;
m is 0, 1, or 2;
R⁶ is selected from the group consisting of C₆-C₁₀ aryl and 5-10 membered heteroaryl, both of which are substituted with 3-10 membered heterocycloalkyl optionally substituted with C₁-C₆ alkyl;
R⁸ is C(O)C₁-C₆ alkyl-OH or SO₂C₃-C₆ cycloalkyl;
R² is selected from the group consisting of: 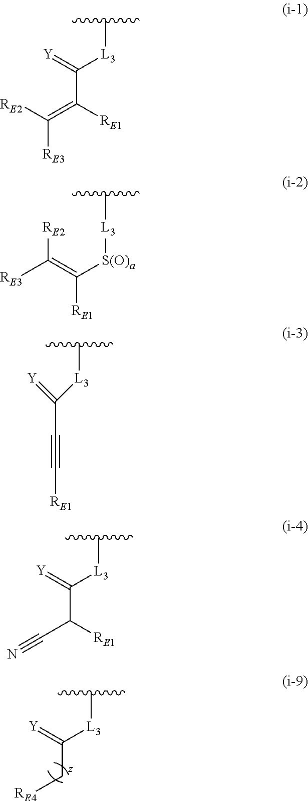 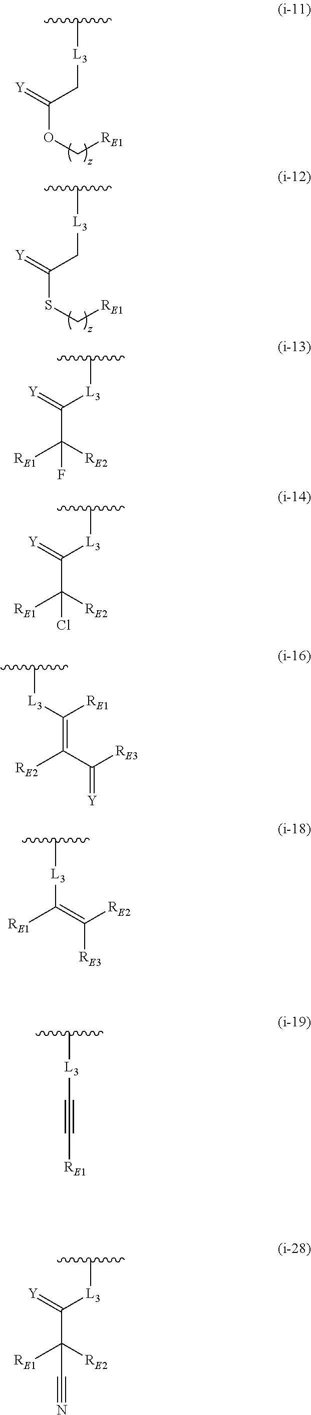 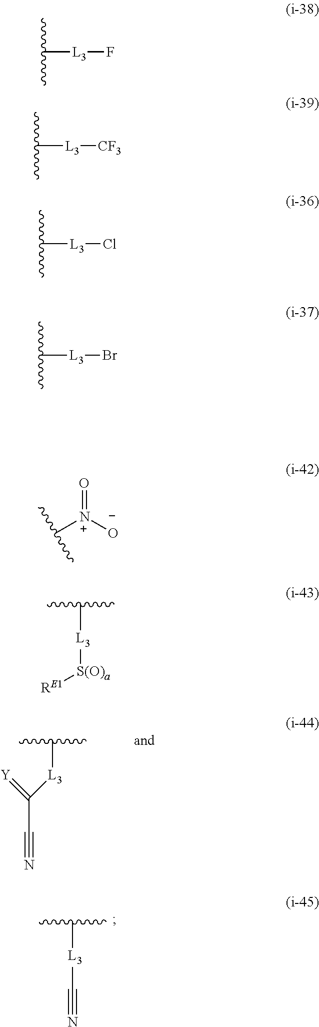
L₃ is a bond, —NH—, —N(C₁-C₄ alkyl)-, or C—C₄ alkylene;
each of R_{E1}, R_{E2}, R_{E3}, and R_{E4} is independently selected from the group consisting of hydrogen, halogen, C₁-C₆ alkyl, C₂-C₆ alkenyl, C₂-C₆ alkynyl, 3-12 membered cycloalkyl, 3-12 membered heterocycloalkyl, 6-12 membered aryl, and 5-12 membered heteroaryl, CN, CH₂OR_{EE}, CH₂N(R_{EE})₂, CH₂SR_{EE}, OR_{EE}, N(R_{EE})₂, SR_{EE}, wherein alkyl, alkenyl, alkynyl, cycloalkyl, heterocyclyl, aryl, and heteroaryl are optionally substituted with one, two, or three R⁹;
or, alternatively, R_{E1} and R_{E3}, or R_{E2} and R_{E3}, or R_{E1} and R_{E2} are joined to form 3-8 membered cycloalkyl or 4-7 membered heterocycloalkyl, both of which are optionally substituted with one, two, or three R⁹;
each R_{EE} is independently selected from the group consisting of hydrogen, C₁-C₆ alkyl, C₁-C₆ alkoxy, C₂-C₆ alkenyl, C₂-C₆ alkynyl, 3-8 membered cycloalkyl, 3-8 membered heterocycloalkyl, 6-10 membered aryl, and 5-10 membered heteroaryl, wherein alkyl, alkenyl, alkynyl, cycloalkyl, heterocyclyl, aryl, and heteroaryl are optionally substituted with one, two, or three R⁹;
or, alternatively, two R_{E}E groups, together with the atom to which they are attached, form 4-7 membered heterocycloalkyl;
each Y is independently O, S, CH₂, or NR_{E7};
R_{E7} is hydrogen, C₁-C₆ alkyl, or a nitrogen protecting group;
each R⁹ is independently selected from the group consisting of halo, OH, NH₂, NH(C₁-C₆ alkyl), and N(C₁-C₆ alkyl)₂;
a is 0, 1, or 2; and
z is 1, 2, or 3.

6. The compound of any one of claims 1-5, wherein the compound is a compound of Formula Ia: 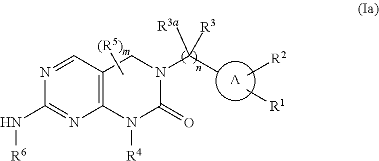
or a pharmaceutically acceptable salt thereof.

7. The compound of any one of claims 1-5, wherein the compound is a compound of Formula Ib: 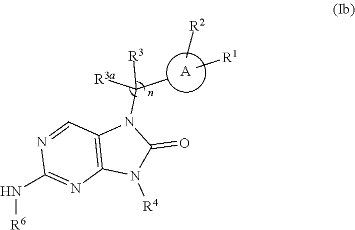
or a pharmaceutically acceptable salt thereof.

8. The compound of any one of claims 1-5, wherein the compound is a compound of Formula Ic: 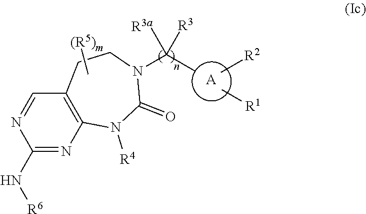
or a pharmaceutically acceptable salt thereof.

9. The compound of any one of claims 1-5, wherein the compound is a compound of Formula Id: 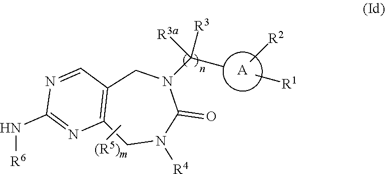
or a pharmaceutically acceptable salt thereof.

10. The compound of any one of claims 1-5, wherein the compound is a compound of Formula Ie: 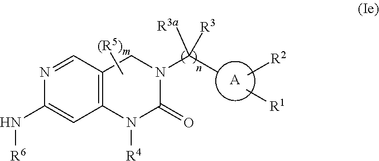
or a pharmaceutically acceptable salt thereof.

11. The compound of any one of claims 1-5, wherein the compound is a compound of Formula If: 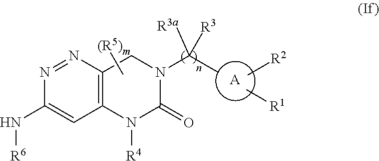
or a pharmaceutically acceptable salt thereof.

12. The compound of any one of claims 1-4, wherein X is C(O), C(S), or SO₂.

13. The compound of any one of claims 1-4 and 12, wherein Y is NH.

14. The compound of any one of claims 1-13, wherein A is selected from the group consisting of C₂-C₆ alkenyl, C₆-C₁₀ aryl, 5-10 membered heteroaryl, 3-10 membered heterocycloalkyl, and 6-10 membered bicyclic ring.

15. The compound of any one of claims 1-14, wherein A is selected from the group consisting of phenyl, 5-6 membered heteroaryl, 5-6 membered heterocycloalkyl, and 9-10 membered bicyclic ring.

16. The compound of any one of claims 1-15, wherein A is selected from the group consisting of phenyl, thiophene, pyrrole, pyridine, pyrrolidine, piperidine, indoline, and tetrahydroquinoline.

17. The compound of any one of claims 1-5, and 12-16, wherein C is pyrimidine.

18. The compound of any one of claims 1-5, and 12-16, wherein C is pyridine.

19. The compound of any one of claims 1-5, and 12-16, wherein C is pyridazine.

20. The compound of any one of claims 1-19, wherein R¹ is selected from the group consisting of H, halo, CN, C₁-C₃ alkyl, C₁-C₃ haloalkyl, 0-C₁-C₃ alkyl, 0(3-4 membered heterocycle), NHSO₂C₁-C₃ alkyl, and NH(3-5 membered heterocycloalkyl).

21. The compound of any one of claims 1-20, wherein
R³ and R^{3a} are each independently H or C₁-C₃ alkyl;
alternatively, R³ and R^{3a} optionally combine to form C₃-C₅ cycloalkyl.

22. The compound of any one of claims 1-20, wherein n is 0.

23. The compound of any one of claims 1-21, wherein n is 1.

24. The compound of any one of claims 1-21, wherein n is 2.

25. The compound of any one of claims 1-24, wherein R⁴ is C₁-C₃ alkyl or 3-6 membered heterocycloalkyl optionally substituted with R¹.

26. The compound of any one of claims 1-25, wherein R⁴ is C₁-C₃ alkyl, pyrrolidine, or piperidine, wherein pyrrolidine and piperidine are optionally substituted with R¹.

27. The compound of any one of claims 1-26, wherein R⁵ is C₁-C₃ alkyl or halo.

28. The compound of any one of claims 1-6 and 8-24, wherein R⁴ and R⁵ combine to form 3-10 membered heterocycloalkyl.

29. The compound of any one of claims 1-5, 12-24, 27, and 28, wherein R⁴ and R⁵ combine with B to form heterobicyclo[2.2.1]heptane or heterobicyclo[3.2.1]octane.

30. The compound of any one of claims 1-6 and 8-26, wherein two R⁵ combine to form C₃-C₁₀ cycloalkyl.

31. The compound of any one of claims 1-6, 8-26, and 29 wherein two R⁵ combine to form C₃-C₆ cycloalkyl.

32. The compound of any one of claims 1-6, and 8-26, wherein m is 0.

33. The compound of any one of claims 1-6, and 8-26, wherein m is 1.

34. The compound of any one of claims 1-6, and 8-31, wherein m is 2.

35. The compound of any one of claims 1-34, wherein R⁶ is selected from the group consisting of phenyl and 5-6 membered heteroaryl, both of which are substituted with 5-6 membered heterocycloalkyl optionally substituted with C₁-C₃ alkyl.

36. The compound of any one of claims 1-34, wherein R⁶ is selected from the group consisting of phenyl, 5-6 membered heteroaryl, and 5-6 membered heterocycloalkenyl all of which are optionally substituted 1, 2, or 3 times with R^{6a}, and wherein each R^{6a} is independently selected from the group consisting of halo, CN, C₁-C₃ alkyl, OC₁-C₃ alkyl, N(C₁-C₃ alkyl)₂, SO₂N(H)(C₁-C₃ alkyl-OH), 6-8 membered heterocycloalkyl, 5-6 membered heterocycloalkenyl, and 5-6 membered heteroaryl, wherein C₁-C₃ alkyl, OC₁-C₃ alkyl, 6-8 membered heterocycloalkyl, 5-6 membered heterocycloalkenyl, and 5-6 membered heteroaryl are optionally substituted with halo, OH, C₁-C₃ alkyl, N(C₁-C₃ alkyl)₂, SO₂N(H)(C₁-C₃ alkyl), and 5-6 membered heterocycloalkyl, wherein 5-6 membered heterocycloalkyl is optionally substituted with C₁-C₃ alkyl.

37. The compound of any one of claims 1-34 and 36, wherein R^{6a} is 6-8 membered heterocycloalkyl and has the following structure: 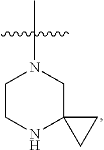
wherein said heterocycloalkyl is optionally substituted with C₁-C₃ alkyl.

38. The compound of any one of claims 1-27 and 30-37, wherein R⁸ is C(O)C₁-C₃ alkyl-OH or SO₂C₃-C₄ cycloalkyl.

39. The compound of any one of claims 1-38, wherein R² is 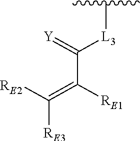
wherein
L³ is a bond, N(H), —N(C₁-C₄ alkyl)-, or C₁-C₄ alkylene;
Y is O, S, or CH₂; and
each of R_{E1}, R_{E2}, and R_{E3}, are independently selected from H, halo, and C₁-C₆ alkyl.

40. The compound of any one of claims 1-39, wherein R² is selected from the group consisting of

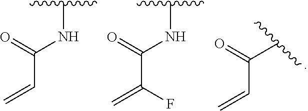

41. The compound of any one of claims 1-39, wherein R² is selected from the group consisting of

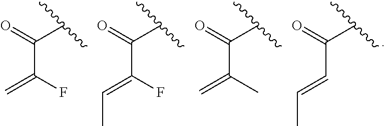

42. The compound of any one of claims 1-38, wherein R² is 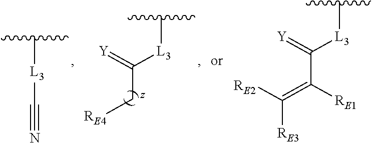
wherein
L³ is a bond;
Y is 0;
z is 1;
each of R_{E1}, R_{E2}, R_{E3}, and R_{E4} are independently selected from CN and C₁-C₆ alkyl wherein C₁-C₆ alkyl is optionally substituted with one R⁹; and
R⁹ is selected from the group consisting of halo and N(C₁-C₆ alkyl)₂.

43. The compound of any one of claims 1-38 and 42, wherein R² is selected from the group consisting of

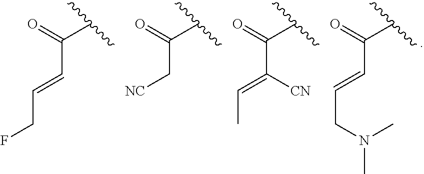

44. The compound of any one of claims 1-43, wherein the compound is selected from the group consisting of a compound in Table 1, or a pharmaceutically acceptable salt thereof.

45. The compound of any one of claims 1-43, wherein the compound of Formula I is selected from the group consisting of a compound in Table 1a, or a pharmaceutically acceptable salt thereof.

46. The compound of any one of claims 1-43, wherein the compound of Formula I is selected from the group consisting of a compound in Table 1 b, or a pharmaceutically acceptable salt thereof.

47. A pharmaceutical composition comprising a compound of any one of claims 1-46, or a pharmaceutically acceptable salt thereof, and a pharmaceutically acceptable carrier.

48. A method of inhibiting the activity of EGFR in a subject in need thereof comprising administering to the subject a therapeutically effective amount of a compound of any one of claims 1-46 or the pharmaceutical composition of claim 47.

49. A method of treating cancer in a subject in need thereof comprising administering to the subject a therapeutically effective amount of a compound of any one of claims 1-46 or the pharmaceutical composition of claim 47.

50. The method of claim 49, wherein the cancer is selected from the group consisting of lung cancer, colon cancer, breast cancer, endometrial cancer, thyroid cancer, glioma, squamous cell carcinoma, and prostate cancer.

51. The method according to claim 49, wherein the cancer is non-small cell lung cancer (NSCLC).
